(19) United States
(12) Patent Application Publication
(10) Pub. No.: US 2009/0265813 A1
(43) Pub. Date: Oct. 22, 2009

(54) STRESS TOLERANCE IN PLANTS

(75) Inventors: Neal I. Gutterson, Oakland, CA (US); Oliver J. Ratcliffe, Oakland, CA (US); T. Lynne Reuber, San Mateo, CA (US); Karen S. Century, Albany, CA (US); Katherine Krolikowski, Richmond, CA (US); Jennifer Costa, Union City, CA (US); Robert A. Creelman, Castro Valley, CA (US); Frederick D. Hempel, Albany, CA (US); Roderick W. Kumimoto, San Bruno, CA (US); Emily L. Queen, San Bruno, CA (US); Peter P. Repetti, Emeryville, CA (US); Luc Adam, Hayward, CA (US)

Correspondence Address: MENDEL BIOTECHNOLOGY C/O MOFO SF, 425 MARKET STREET, SAN FRANCISCO, CA, 94105, US

(73) Assignee: Mendel Biotechnology , Inc., Hayward, CA (US)

(21) Appl. No.: 12/064,961

(22) PCT Filed: Aug. 31, 2006
(86) PCT No.: PCT/US2006/034615

§ 371 Date: Dec. 22, 2008

Related U.S. Application Data
(60) Provisional application No. 60/713,952, filed on Aug. 31, 2005.

(51) Int. Cl. C12N 15/82; A01H 5/00

(52) U.S. Cl. 800/290; 800/298; 800/278

(57) ABSTRACT

Transcription factor polynucleotides and polypeptides incorporated into expression vectors have been introduced into plants and were ectopically expressed. Transgenic plants transformed with many of these expression vectors have been shown to be more resistant to disease (in some cases, to more than one pathogen), or more tolerant to an abiotic stress (in some cases, to more than one abiotic stress). The abiotic stress may include salt, hyperosmotic stress, heat, cold, drought, or low nitrogen conditions.

### ACKNOWLEDGEMENT

This invention was supported in part by NSF SBIR grants DMI-0215130, DMI-0320074, and DMI-0349577. The U.S. government may have certain rights in this invention.

### JOINT RESEARCH AGREEMENT

The claimed invention, in the field of functional genomics and the characterization of plant genes for the improvement of plants, was made by or on behalf of Mendel Biotechnology, Inc. and Monsanto Corporation as a result of activities undertaken within the scope of a joint research agreement, and in effect on or before the date the claimed invention was made.

### FIELD OF THE INVENTION

The present invention relates to plant genomics and plant improvement.

### BACKGROUND OF THE INVENTION

Abiotic stress and yield. In the natural environment, plants often grow under unfavorable conditions, such as drought (low water availability), salinity, chilling, freezing, high temperature, flooding, or strong light. Any of these abiotic stresses can delay growth and development, reduce productivity, and in extreme cases, cause the plant to die. Enhanced tolerance to these stresses would lead to yield increases in conventional varieties and reduce yield variation in hybrid varieties. Of these stresses, low water availability is a major factor in crop yield reduction worldwide.

Water deficit is a common component of many plant stresses. Water deficit occurs in plant cells when the whole plant transpiration rate exceeds the water uptake. In addition to drought, other stresses, such as salinity and low temperature, produce cellular dehydration (McCue and Hanson, 1990).

Salt (and drought) stress signal transduction consists of ionic and osmotic homeostasis signaling pathways. The ionic aspect of salt stress is signaled via the SOS pathway where a calcium-responsive SOS3-SOS2 protein kinase complex controls the expression and activity of ion transporters such as SOS1. The pathway regulating ion homeostasis in response to salt stress has been reviewed recently by Xiong and Zhu (2002a).

The osmotic component of salt-stress involves complex plant reactions that are possibly overlapping with drought- and/or cold-stress responses. Common aspects of drought-, cold- and salt-stress response have been reviewed by Xiong and Zhu (2002). These include:

Abscisic acid (ABA) biosynthesis is regulated by osmotic stress at multiple steps. Both ABA-dependent and -independent osmotic stress signaling first modify constitutively expressed transcription factors, leading to the expression of early response transcriptional activators, which then activate downstream stress tolerance effector genes.

Based on the commonality of many aspects of cold, drought, and salt stress responses, it can be concluded that genes that increase tolerance to cold or salt stress can also improve drought stress protection. In fact, this has already been demonstrated for transcription factors (in the case of AtCBF/DREB1) and for other genes such as OsCDPK7 (Saijo et al. (2000)), or AVP1 (a vacuolar pyrophosphatase-proton-pump, Gaxiola et al. (2001)).

Heat stress often accompanies conditions of low water availability. Heat itself is seen as an interacting stress and adds to the detrimental effects caused by water deficit conditions. Evaporative demand exhibits near exponential increases with increases in daytime temperatures and can result in high transpiration rates and low plant water potentials (Hall et al. (2000)). High-temperature damage to pollen almost always occurs in conjunction with drought stress, and rarely occurs under well-watered conditions. Thus, separating the effects of heat and drought stress on pollination is difficult. Combined stress can alter plant metabolism in novel ways; therefore, understanding the interaction between different stresses may be important for the development of strategies to enhance stress tolerance by genetic manipulation.

Plant pathogens and impact on yield. While a number of plant pathogens exist that may significantly impact yield or affect the quality of plant products, specific attention is being given in this application to a small subset of these microorganisms. These include:

Sclerotinia. Sclerotinia sclerotiorum is a necrotrophic ascomycete that causes destructive rots of numerous plants (Agrios (1997)). Sclerotinia stem rot is a significant pathogen of soybeans in the northern U.S. and Canada.

Botrytis. Botrytis causes blight or gray mold, a disease of plants that infects a wide array of herbaceous annual and perennial plants. Environmental conditions favorable to this pathogen can significantly impact ornamental plants, vegetables and fruit. Botrytis infections generally occur in spring and summer months following cool, wet weather, and may be particularly damaging when these conditions persist for several days.

Fusarium. Fusarium or vascular wilt may affect a variety of plant host species. Seedlings of developing plants may be infected with Fusarium, resulting in the grave condition known as “damping-off”. Fusarium species also cause root, stem, and corn rots of growing plants and pink or yellow molds of fruits during post-harvest storage. The latter affect ornamentals and vegetables, particularly root crops, tubers, and bulbs.

Drought-Disease Interactions. Plant responses to biotic and abiotic stresses are governed by complex signal transduction networks. There appears to be significant interaction between these networks, both positive and negative. An understanding of the complexity of these interactions will be necessary to avoid unintended consequences when altering plant signal transduction pathways to engineer drought or disease resistance.

Physiological interactions between drought and disease. The majority of plant pathogenic fungi are more problematic in wet conditions. Most fungi require free water on the plant surface or high humidity for spores to germinate and successfully invade host tissues (Agrios (1997)). Therefore, overall disease pressure is generally lower in dry conditions. However, there are exceptions to this pattern. Water stress can increase the incidence of certain facultative pathogens such as root rots, stem rots, and stem cankers (reviewed in Boyer (1995)). Some examples of diseases that are more prevalent or severe in drought conditions are Fusarium root rot and common root rot (Bipolaris sorokiniana) of wheat, corn smut, and root rot and charcoal rot of soybeans (North Dakota State Extension Service 2002, 2004). Vulnerability to pathogens may be increased when water stress decreases available photosynthate and therefore energy to synthesize defensive compounds (Boyer (1995)). The increased damage caused by root rots in dry weather may also reflect the inability of the plant to tolerate as much root damage under dry conditions as under ample water. Increasing crop drought tolerance may decrease vulnerability to these diseases.

Transcription factors (TFs) and other genes involved in both abiotic and biotic stress resistance. Despite the evidence for negative cross-talk between drought and disease response pathways, a number of genes have been shown to function in both pathways, indicating possible convergence of the signal transduction pathways. There are numerous example of genes that are inducible by multiple stresses. For instance, a global TxP analysis revealed classes of transcription factor that are mainly induced by abiotic stresses or disease, but also a class of transcription factors induced both by abiotic stress and bacterial infection (Chen et al. (2002a)).

Implications for crop improvement. Plant responses to drought and disease interact at a number of levels. Although dry conditions do not favor most pathogens, plant defenses may be weakened by metabolic stress or hormonal cross-talk, increasing vulnerability to pathogens that can infect under drought conditions. However, there is also evidence for convergence of abiotic and biotic stress response pathways, based on genes that confer tolerance to multiple stresses. Given our incomplete understanding of these signaling interactions, plants with positive alterations in one stress response should be examined carefully for possible alterations in other stress responses.

### SUMMARY OF THE INVENTION

The present invention pertains to transcription factor polynucleotides and polypeptides, and expression vectors that comprise these sequences. A significant number of these sequences have been incorporated into expression vectors that have been introduced into plants, thus allowing for the polypeptides to be ectopically expressed. These sequences include polynucleotide sequences 1 to 2n−1, where n=1 to 210, and polypeptide sequences 1 to 2n, where n=1 to 210. The expression vector comprises a constitutive, an inducible or a tissue-specific promoter operably linked to the polynucleotide sequence of the expression vector. Transgenic plants transformed with many of these expression vectors have been shown to be more resistant to disease (and in some cases, to more than one pathogen), or more tolerant to an abiotic stress (and in some cases, to more than one abiotic stress). The abiotic stress may include salt, hyperosmotic stress, heat, cold, drought, or low nitrogen conditions.

Alternatively, the expression vector may comprise a polynucleotide that encodes a transcription factor polypeptide sequence fused to a GAL4 activation domain, thus creating either a C-terminal or an N-terminal GAL4 activation domain protein fusion. Using a number of the sequences of the invention, these constructs have also been shown to confer disease resistance or abiotic stress tolerance when the plants express the fusion protein.

Transgenic plants that are transformed with these expression vectors, and seed produced by these transgenic plants that comprise any of the sequences of the invention, are also encompassed by the invention.

The invention is also directed to methods for increasing the yield of a plant growing in conditions of stress, as compared to a wild-type plant of the same species growing in the same conditions of stress. In this case, the plant is transformed with a polynucleotide sequence encoding a transcription factor polypeptide of the invention, where the polynucleotide is operably linked to a constitutive, inducible or tissue-specific promoter. The transformed plant that ectopically expresses the transcription factor polypeptide is then selected, and this plant may have greater yield than a wild-type plant of the same species (that is, a non-transformed plant), when the transformed plant is grown in conditions of salt, hyperosmotic stress, heat, cold, drought, low nitrogen, or disease stress.

### BRIEF DESCRIPTION OF THE SEQUENCE LISTING AND DRAWINGS

The Sequence Listing provides exemplary polynucleotide and polypeptide sequences of the invention. The traits associated with the use of the sequences are included in the Examples.

CD-ROMs Copy 1—Sequence Listing Part, Copy 2—Sequence Listing Part, Copy 3—Sequence Listing Part, and the CRF copy of the Sequence Listing, all filed under PCT Administrative Instructions §801(a), are read-only memory computer-readable compact discs. Each contains a copy of the Sequence Listing in ASCII text format. The Sequence Listing is named “MBI0061PCT.ST25.txt”, was created on 28 Aug., 2006, and is 1,587 kilobytes in size. The copies of the Sequence Listing on the CD-ROM discs are hereby incorporated by reference in their entirety.

FIG. 1 shows a conservative estimate of phylogenetic relationships among the orders of flowering plants (modified from Soltis et al. (1997)). Those plants with a single cotyledon (monocots) are a monophyletic clade nested within at least two major lineages of dicots; the eudicots are further divided into rosids and asterids. Arabidopsis is a rosid eudicot classified within the order Brassicales; rice is a member of the monocot order Poales. FIG. 1 was adapted from Daly et al. (2001).

FIG. 2: Phylogenetic tree of CAAT family proteins. There are three main sub-classes within the family: the HAP2 (also known as the NF-YA subclass), HAP3 (NF-YB subclass) and HAP5 (NF-YC subclass) related proteins. Three additional proteins were identified that did not clearly cluster with any of the three main groups and we have designated these as HAP-like proteins. G620, SEQ ID NO: 358, corresponds to LEAFY COTYLEDON 1 (LEC1; Lotan et al., 1998) and G1821, corresponds to LEAFY COTYLEDON 1-LIKE (L1L; Kwong et al., 2003). Other sequences shown in this tree include G1364 (SEQ ID NO: 14), G2345 (SEQ ID NO: 22), G481 (SEQ ID NO: 2), G482 (SEQ ID NO: 28), G485 (SEQ ID NO: 18), G1781 (SEQ ID NO: 56), G1248 (SEQ ID NO: 360), G486 (SEQ ID NO: 356), G484 (SEQ ID NO: 354), G2631 (SEQ ID NO: 362), G1818 (SEQ ID NO: 404), G1836 (SEQ ID NO: 48), G1820 (SEQ ID NO: 44), G489 (SEQ ID NO: 46), G3074 (SEQ ID NO: 410), G1334 (SEQ ID NO: 54), G926 (SEQ ID NO: 52), and G928 (SEQ ID NO: 400). The tree was based on a ClustalW alignment of fall-length proteins using Mega 2 software (protein sequences are provided in the Sequence Listing).

In FIGS. 3A-3F, the alignments of G481, G482, G485, G1364, G2345, G1781 and related sequences are presented. These sequences from Arabidopsis (At) are shown aligned with soybean (Gm), rice (Os) and corn (Zm) sequences with the B domains indicated by the large box that spans FIGS. 3B through 3C. The vertical line to the left in each page of the alignment indicates G482 clade members.

FIG. 4 is a phylogenetic tree of G682-related polypeptide sequences from Arabidopsis thaliana (At), rice (Os), maize (Zm) and soybean (Gm). The tree was based on a ClustalW alignment of full-length proteins using Mega 2 software (protein sequences are provided in the Sequence Listing). The arrow indicates the node identifying an ancestral sequence, from which sequences with related functions to G682 were descended. Sequences shown in this tree include G1816 (SEQ ID NO: 76), G3930 (SEQ ID NO: 412), G226 (SEQ ID NO: 62), G3450 (SEQ ID NO: 74), G2718 (SEQ ID NO: 64), G682 (SEQ ID NO: 60), G3392 (SEQ ID NO: 72), G3393 (SEQ ID NO: 66), G3431 (SEQ ID NO: 68), G3444 (SEQ ID NO: 70), G3448 (SEQ ID NO: 80), G3449 (SEQ ID NO: 78), G3446 (SEQ ID NO: 82), G3445 (SEQ ID NO: 84), G3447 (SEQ ID NO: 86), and G676 (SEQ ID NO: 350).

FIGS. 5A and 5B show the conserved domains making up the DNA binding domains of G682-like proteins from Arabidopsis, soybean, rice, and corn. G682 and its paralogs and orthologs are almost entirely composed of a single repeat MYB-related DNA binding domain that is highly conserved across plant species. The polypeptide sequences within the box are representatives of the G682 clade. Residues making up the consensus sequence appear as boldface text. Sequences shown in this alignment include G214 (SEQ ID NO: 346), G1816 (SEQ ID NO: 76), CPC (CAPRICE; Wada et al. (1997)), G226 (SEQ ID NO: 62), G3450 (SEQ ID NO: 74), G2718 (SEQ ID NO: 64), G682 (SEQ ID NO: 60), G3392 (SEQ ID NO: 72), G3393 (SEQ ID NO: 66), G3431 (SEQ ID NO: 68), G3444 (SEQ ID NO: 70), G3448 (SEQ ID NO: 80), G3449 (SEQ ID NO: 78), G3446 (SEQ ID NO: 82), G3447 (SEQ ID NO: 86), G3445 (SEQ ID NO: 84), and G676 (SEQ ID NO: 350).

FIG. 6 depicts a phylogenetic tree of several members of the RAV family, identified through BLAST analysis of proprietary (using corn, soy and rice genes) and public data sources (all plant species). This tree was generated as a Clustal X 1.81 alignment: MEGA2 tree, Maximum Parsimony, bootstrap consensus. Sequences that are closely related to G867 are considered as being those proteins descending from the node of the tree, indicated by the arrow, with a bootstrap value of 100, bounded by G3451 and G3432 (the clade is indicated by the large box). Sequences shown in this tree include G3451 (SEQ ID NO: 108), G3452 (SEQ ID NO: 98), G3453 (SEQ ID NO: 100), G867 (SEQ ID NO: 88), G1930 (SEQ ID NO: 92), G9 (SEQ ID NO: 106), G993 (SEQ ID NO: 90), G3388 (SEQ ID NO: 110), G3389 (SEQ ID NO: 104), G3390 (SEQ ID NO: 112), G3391 (SEQ ID NO: 94), G3432 (SEQ ID NO: 102), G2690 (SEQ ID NO: 382), and G2687 (SEQ ID NO: 380).

FIGS. 7A-7H show an alignment of AP2 transcription factors from Arabidopsis, soybean, rice and corn. The AP2 domains of these sequences are indicated by the box and the right angle arrow “ [symbol] ” spanning FIGS. 7B to 7C, the “DML motifs” are indicated by box and the downward arrow “↓” spanning FIGS. 7C to 7D, and the B3 domains are indicated by the box and the right angle arrow “ [symbol] ” spanning FIGS. 7D to 7F. Sequences shown in this alignment include G3391 (SEQ ID NO: 94), G3432 (SEQ ID NO: 102), G3390 (SEQ ID NO: 92), G3389 (SEQ ID NO: 104), G3388 (SEQ ID NO: 110), G867 (SEQ ID NO: 88), G1930 (SEQ ID NO: 92), G993 (SEQ ID NO: 90), G9 (SEQ ID NO: 106), G3455 (SEQ ID NO: 96), G3451 (SEQ ID NO: 108), G3452 (SEQ ID NO: 98), G3453 (SEQ ID NO: 100), G2687 (SEQ ID NO: 380), and G2690 (SEQ ID NO: 382).

FIG. 8 compares the B3 domain from the four boxed RAV1 paralogs (G867, G1930, G9, and G993) with the B3 domains from ABI3 related proteins: ABI3 (G621), FUSCA3 (G1014), and LEC2 (G3035). G867 corresponds to SEQ ID NO: 88, G1930 is SEQ ID NO: 92, G9 is SEQ ID NO: 106, G993 is SEQ ID NO: 90, G621 is SEQ ID NO: 376, G1014 is SEQ ID NO: 378, G3035 is SEQ ID NO: 384, and the consensus sequence of the RAV1 B3 domain is SEQ ID NO: 938.

FIG. 9 represents a G1073 Phylogenetic Analysis. A phylogenetic tree and multiple sequence alignments of G1073 and related full length proteins were constructed using ClustalW (CLUSTAL W Multiple Sequence Alignment Program version 1.83, 2003) and MEGA2 (http://www.megasoftware.net) software. ClustalW multiple alignment parameters were as follows:

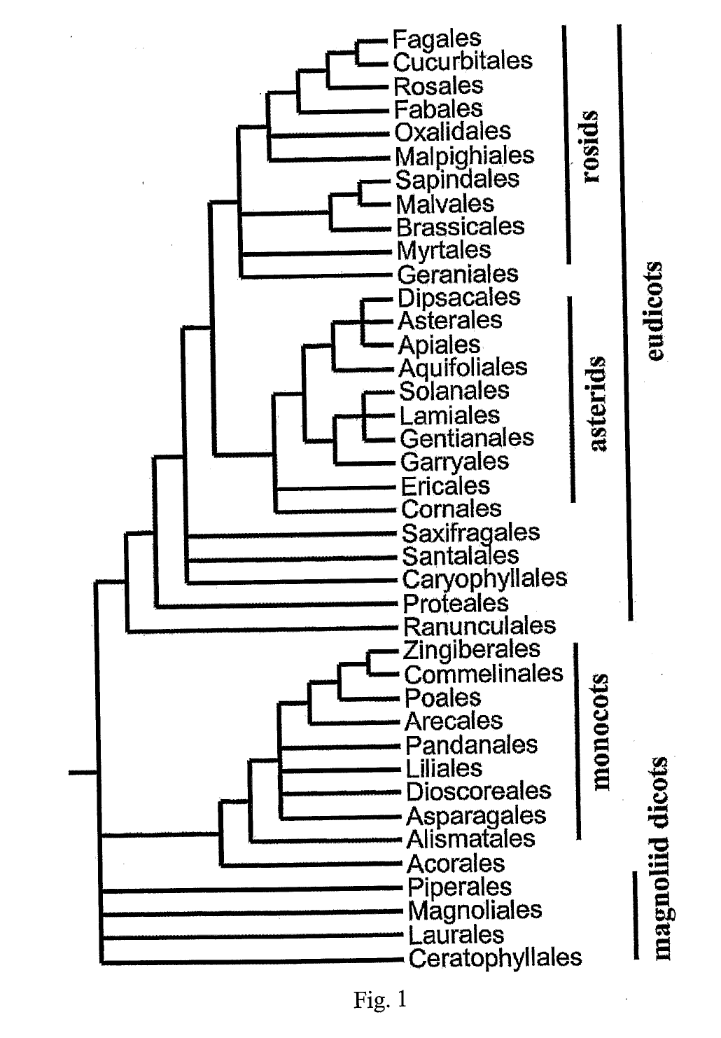

Gap Opening Penalty: 10.00; Gap Extension Penalty: 0.20; Delay divergent sequences: 30%; DNA Transitions Weight: 0.50; Protein weight matrix: Gonnet series; DNA weight matrix: IUB; Use negative matrix: OFF

A FastA formatted alignment was then used to generate a phylogenetic tree in MEGA2 using the neighbor joining algorithm and a p-distance model. A test of phylogeny was done via bootstrap with 1000 replications and Random Seed set to default. Cut off values of the bootstrap tree were set to 50%. Members of the G1073 clade in the large box are considered as being those proteins within the node of the tree below with a bootstrap value of 99, bounded by G2789 and the sequence between G3401 and G3408. Sequences shown in this tree include G2789 (SEQ ID NO: 372), G3407 (SEQ ID NO: 134), G3406 (SEQ ID NO: 116), G3459 (SEQ ID NO: 122), G3460 (SEQ ID NO: 126), G1667 (SEQ ID NO: 128), G1073 (SEQ ID NO: 114), G1067 (SEQ ID NO: 120), G2156 (SEQ ID NO: 130), G3399 (SEQ ID NO: 118), G3400 (SEQ ID NO: 124), G2157 (SEQ ID NO: 144), G3556 (SEQ ID NO: 142), G3456 (SEQ ID NO: 132), G2153 (SEQ ID NO: 138), G1069 (SEQ ID NO: 140), G3401 (SEQ ID NO: 136), and G3408 (SEQ ID NO: 146).

In FIGS. 10A-10H, Clustal W (CLUSTAL W Multiple Sequence Alignment Program version 1.83, 2003) alignments of a number of AT-hook proteins are shown, and include clade members from Arabidopsis (e.g., G1067, G1069, G1073, G1667, G2153, G2156, G2789), soy (e.g., G3456, G3459, G3460), and rice (e.g., G3399, G3400, G3401, G3407) that have been shown to confer similar traits in plants when overexpressed (closely related polypeptides are indicated by vertical line). Also shown are the AT-hook conserved domains (indicated by the right-angled arrow: [symbol] in FIG. 10C) and the second conserved domains indicated by the right-angled arrow [symbol] spanning FIGS. 10D through 10F). Sequences shown in this alignment include G2789 (SEQ ID NO: 372), G3460 (SEQ ID NO: 126), G3459 (SEQ ID NO: 122), G3406 (SEQ ID NO: 116), G3407 (SEQ ID NO: 134), G1069 (SEQ ID NO: 140), G2153 (SEQ ID NO: 138), G3456 (SEQ ID NO: 132), G3401 (SEQ ID NO: 136), G2157 (SEQ ID NO: 144), G3556 (SEQ ID NO: 142), G1067 (SEQ ID NO: 120), G2156 (SEQ ID NO: 130), G3400 (SEQ ID NO: 124), G3399 (SEQ ID NO: 118), and G1073 (SEQ ID NO: 114), G3408 (SEQ ID NO: 146).

FIGS. 11A and 11B show the AP2 domains of ERF transcription factors and the characteristic A and D residues present in the AP2 domain (adapted from Sakuma et al., 2002). Sequences shown in this alignment include G28 (SEQ ID NO: 148), G1006 (SEQ ID NO: 152), G22 (SEQ ID NO: 172), G1004 (SEQ ID NO: 388), G1792 (SEQ ID NO: 222), G1266 (SEQ ID NO: 254), G1752 (SEQ ID NO: 402), G1791 (SEQ ID NO: 230), G1795 (SEQ ID NO: 224), and G30 (SEQ ID NO: 226).

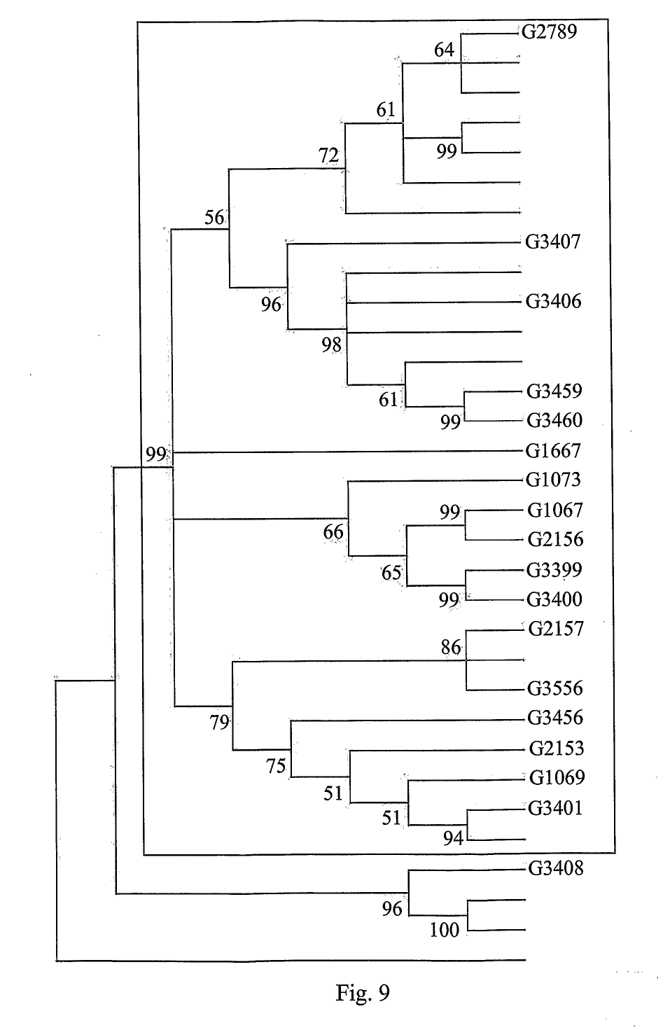

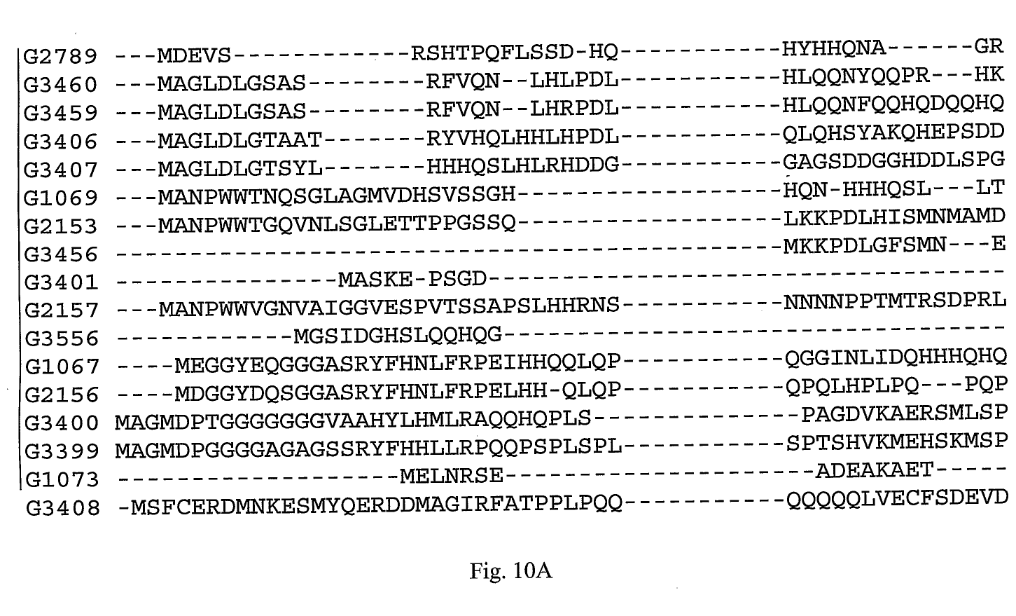

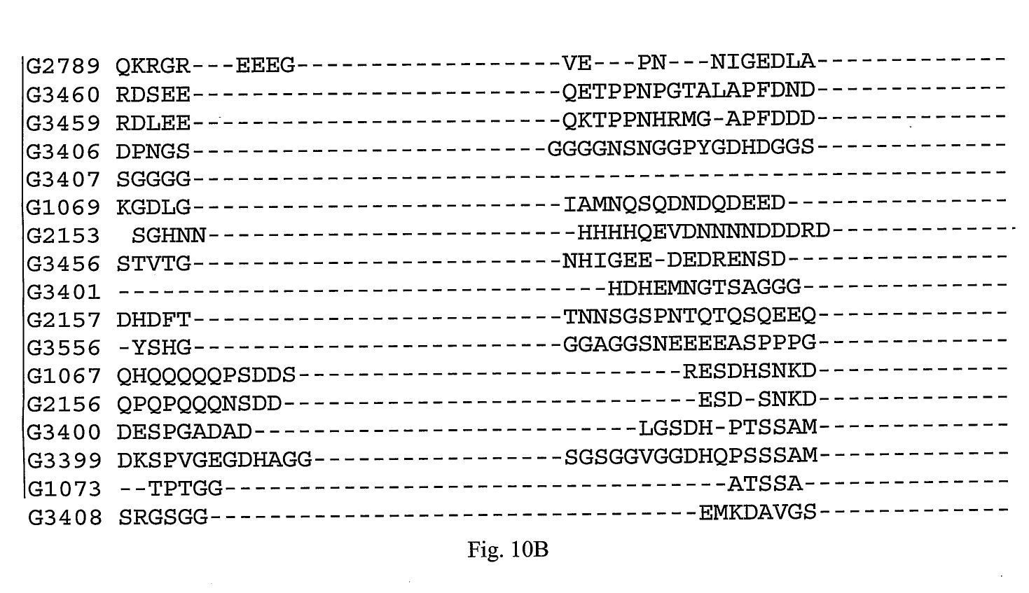

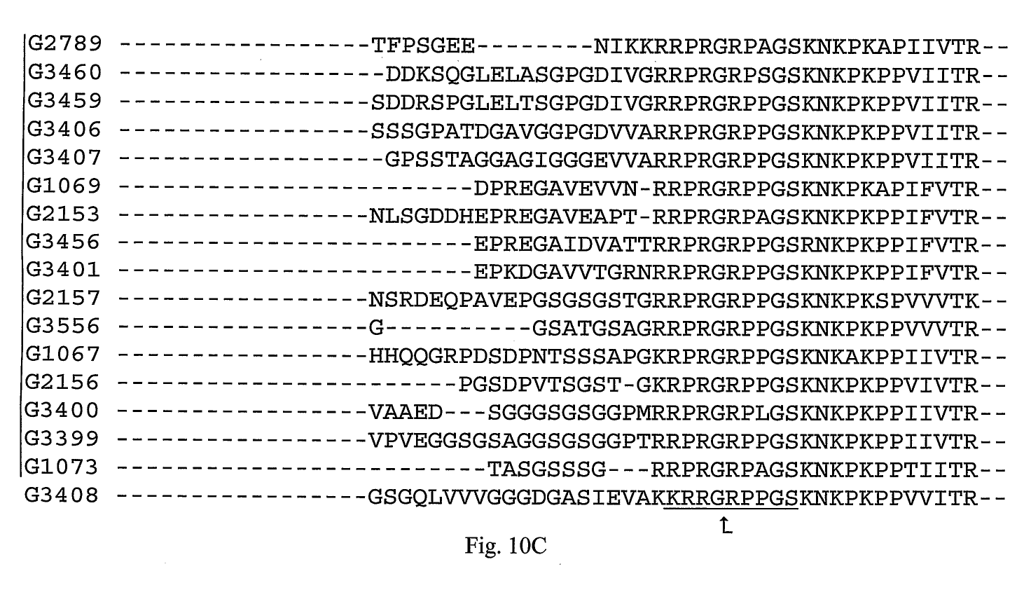

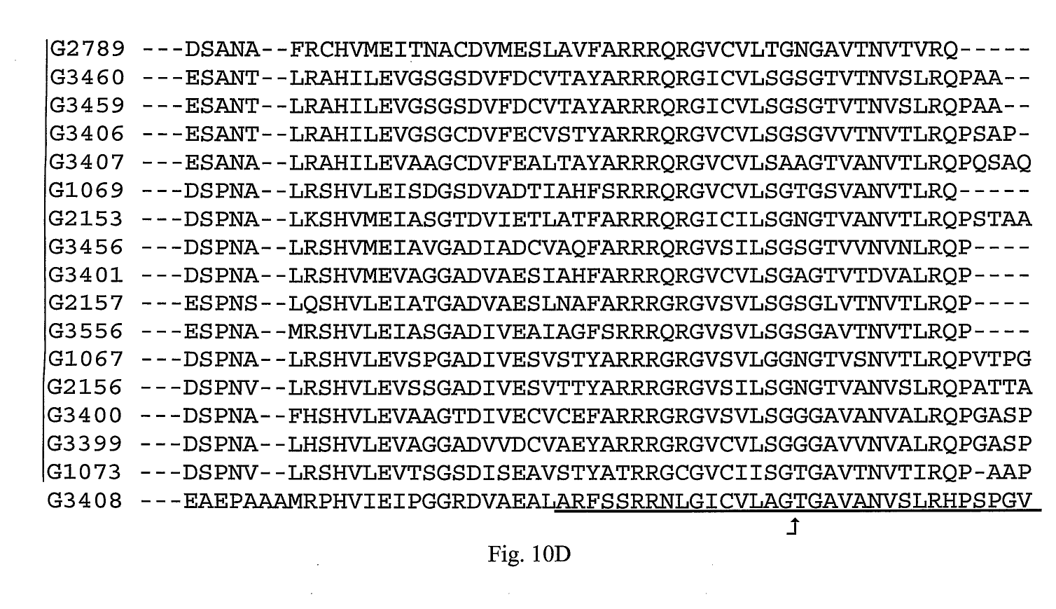

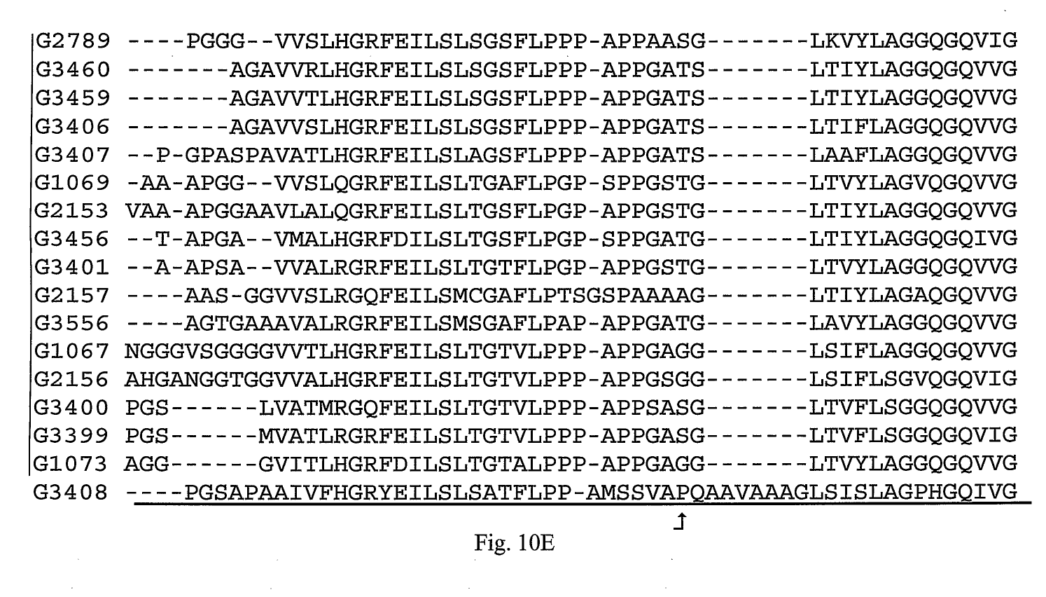

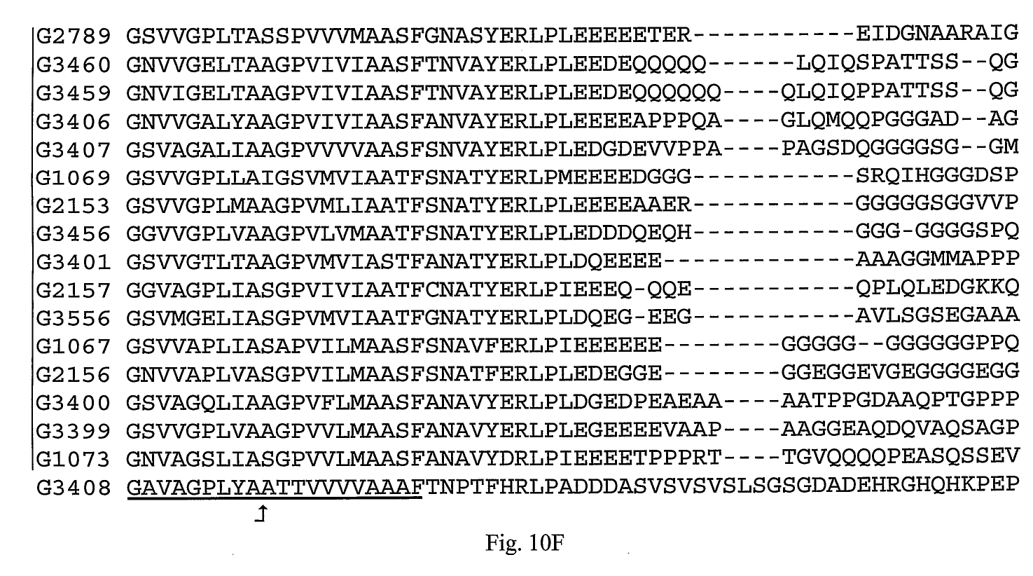

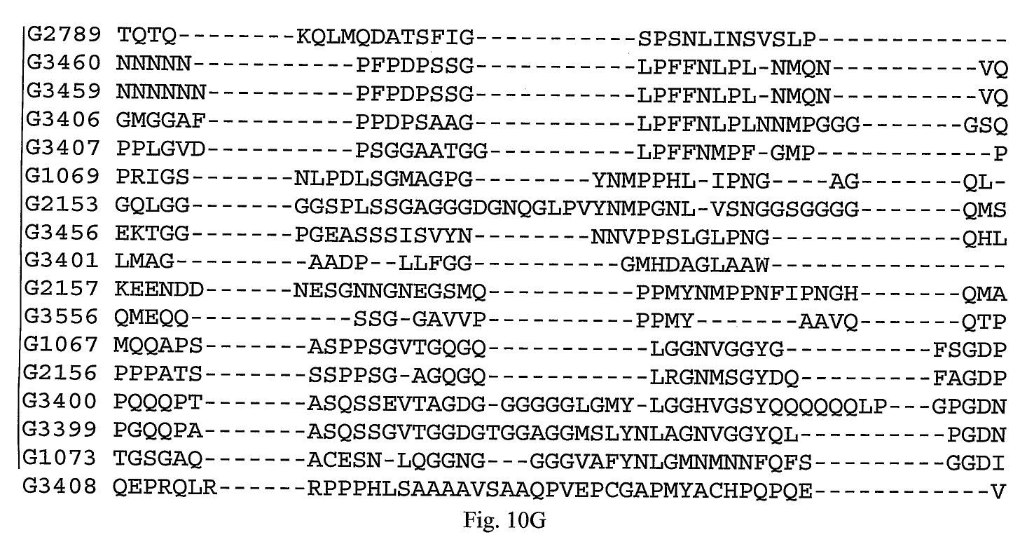

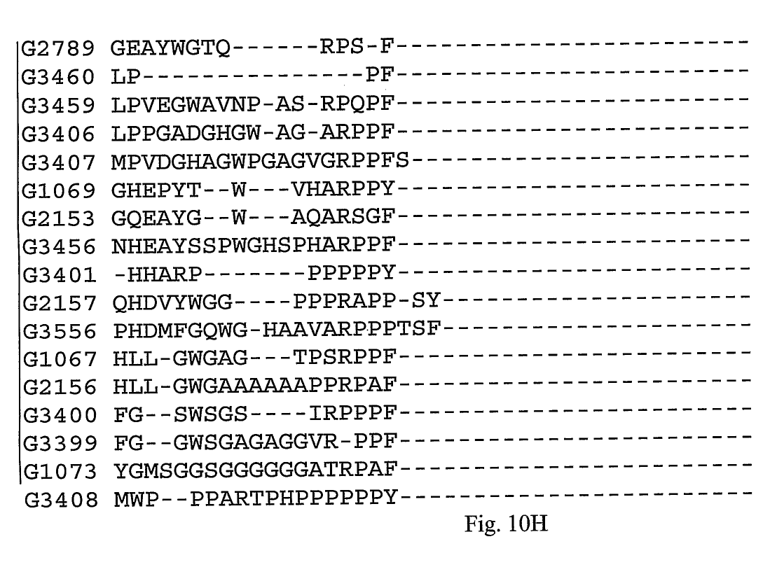

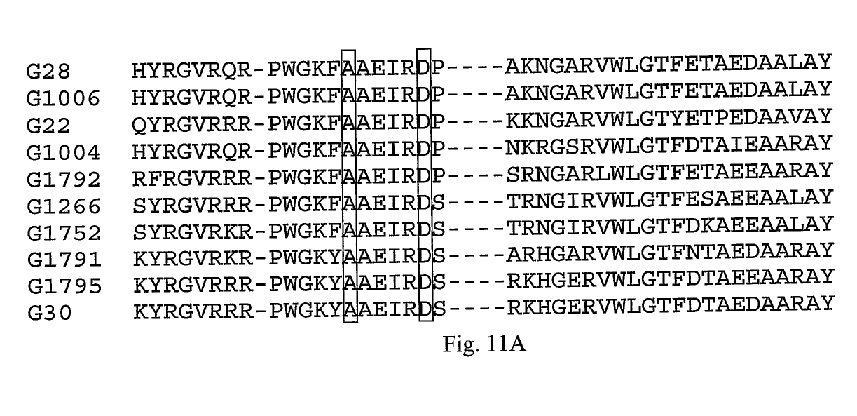

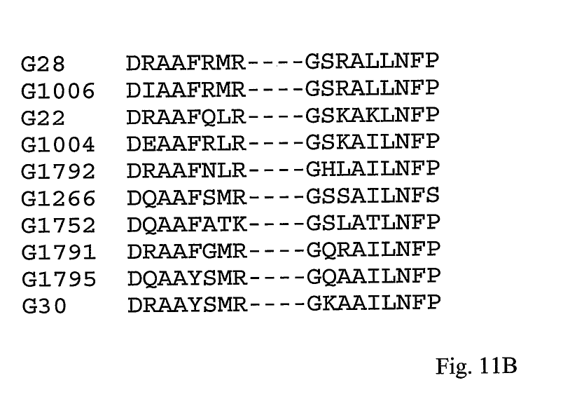

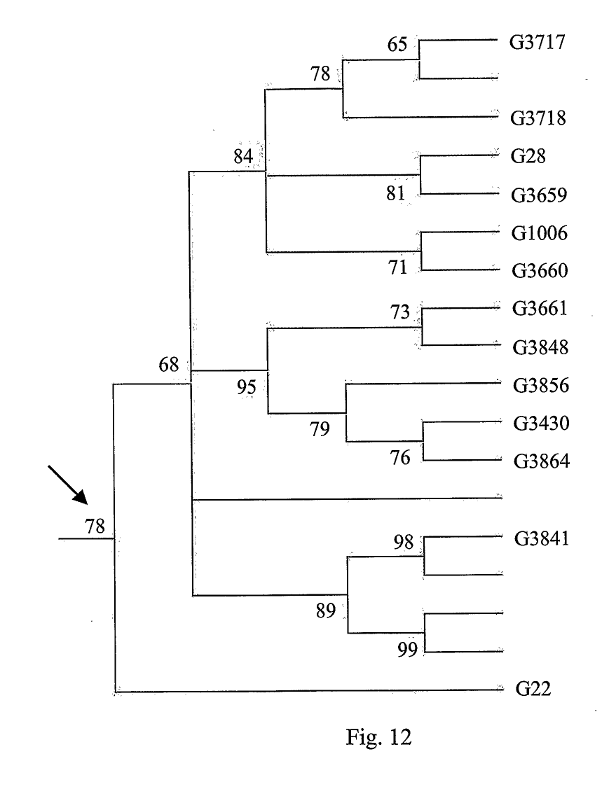

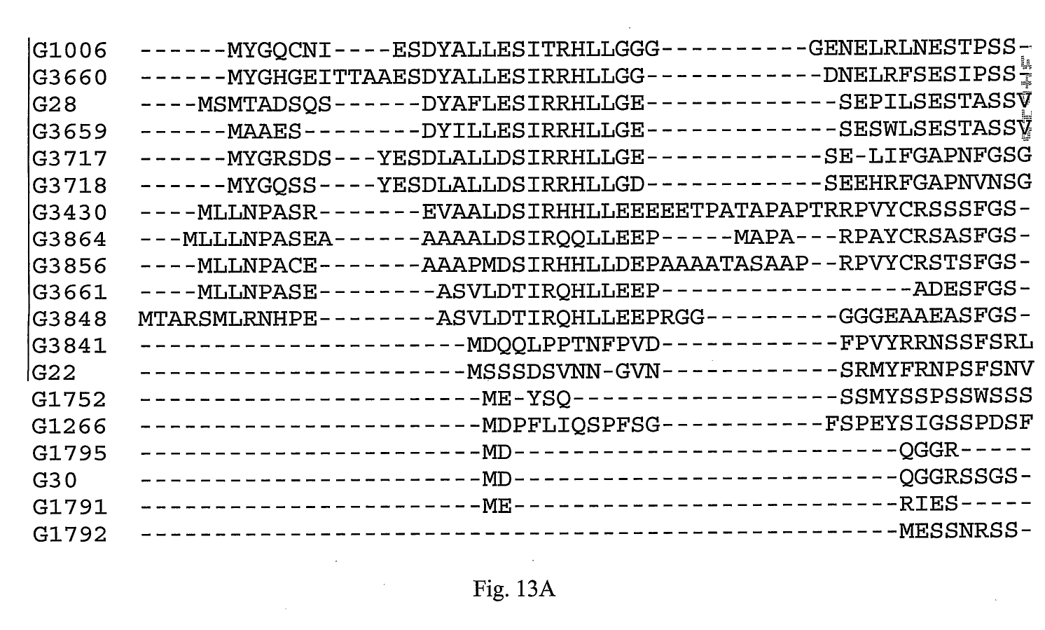

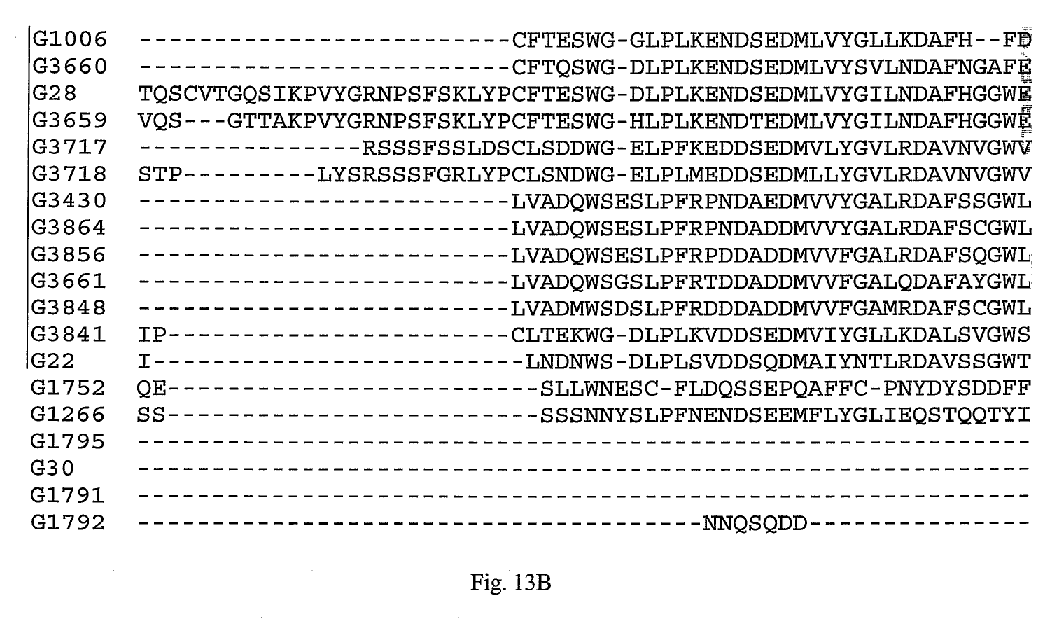

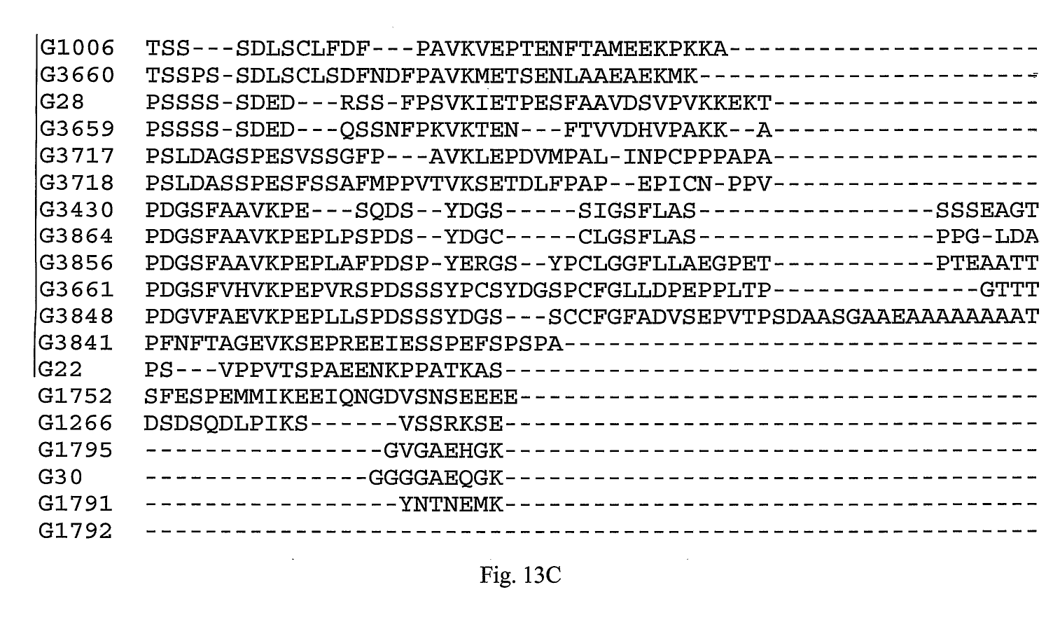

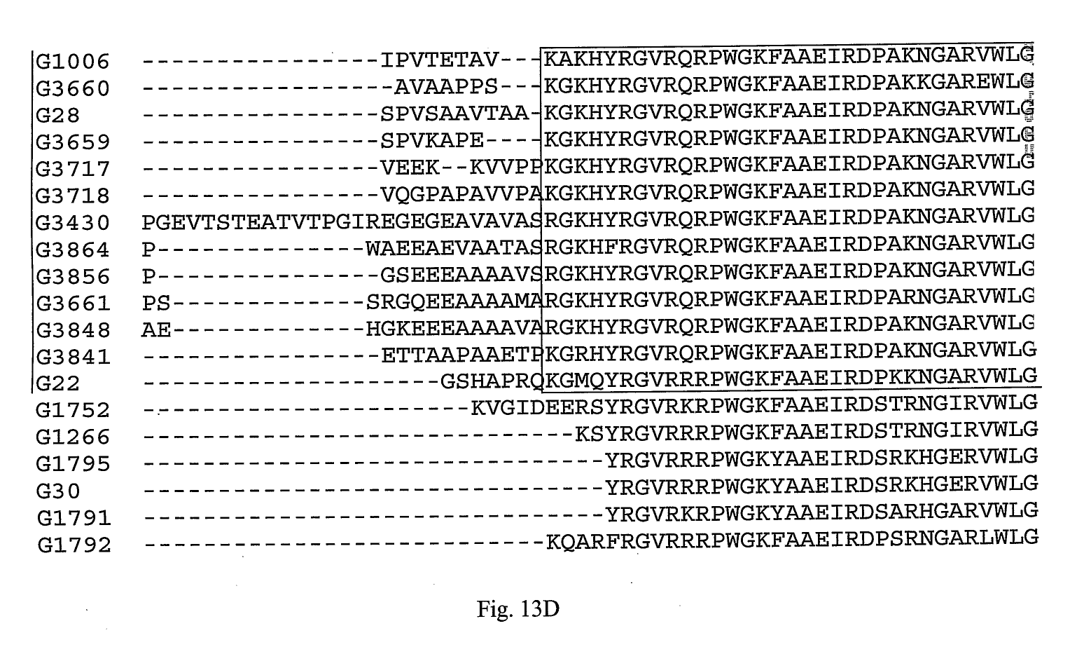

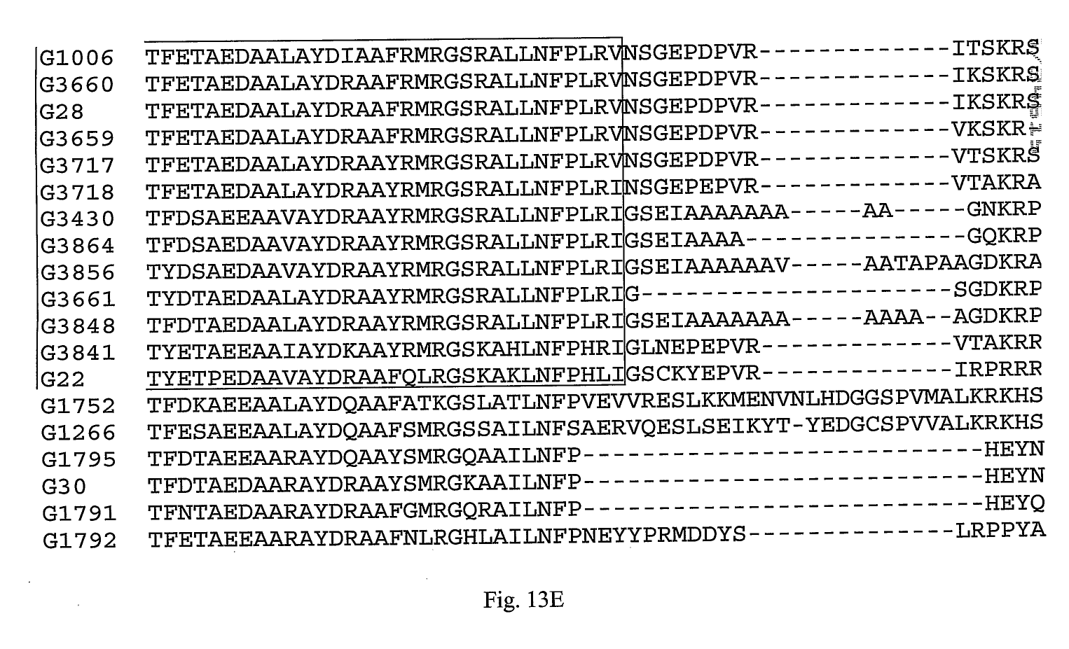

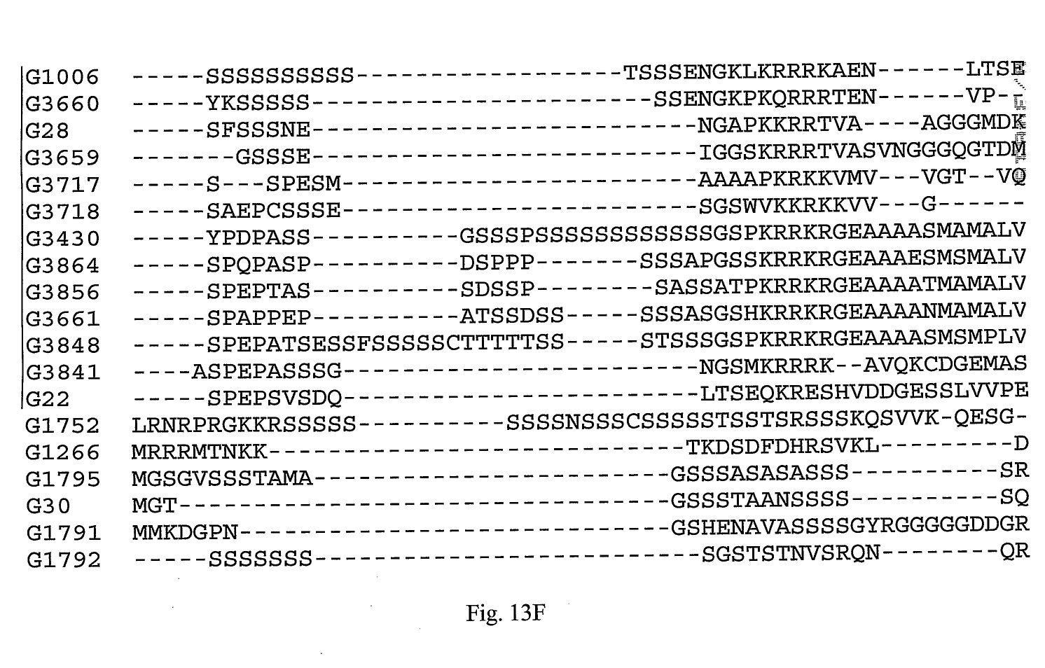

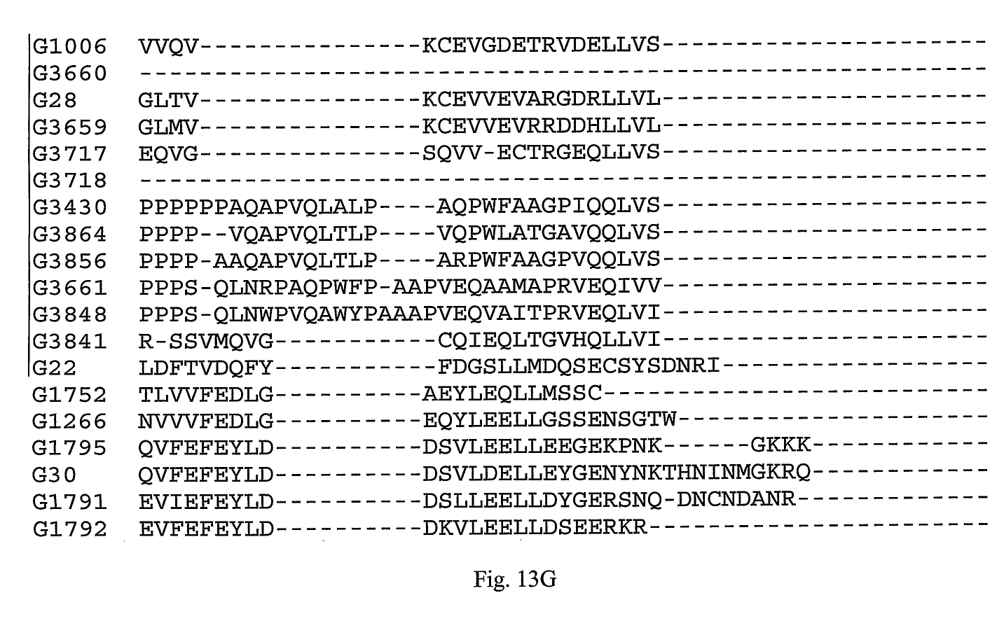

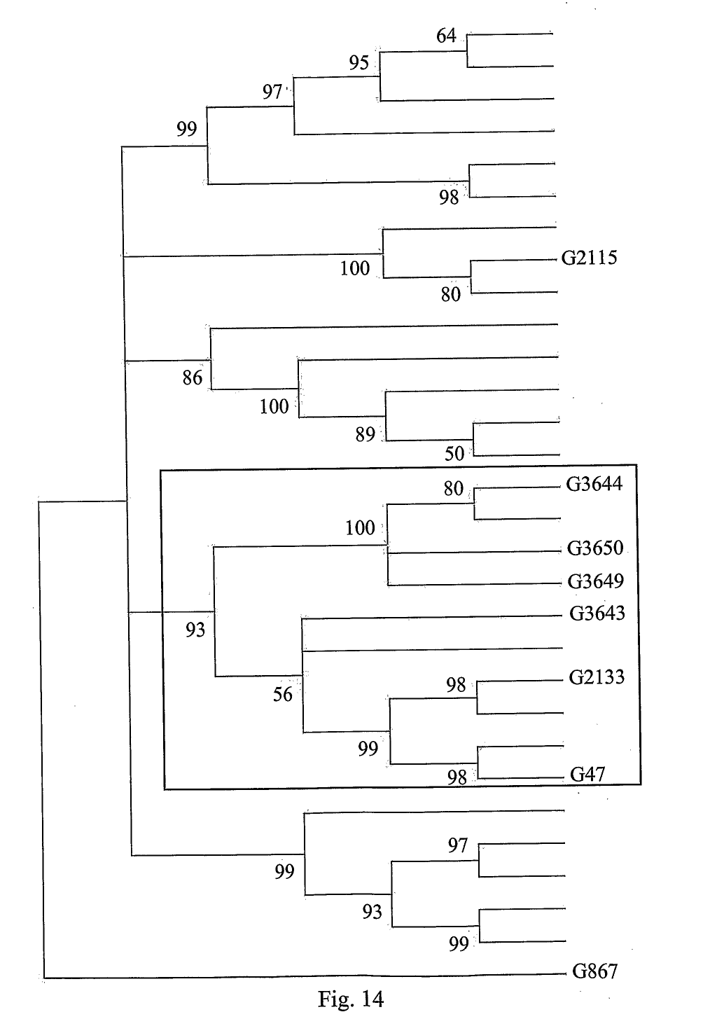

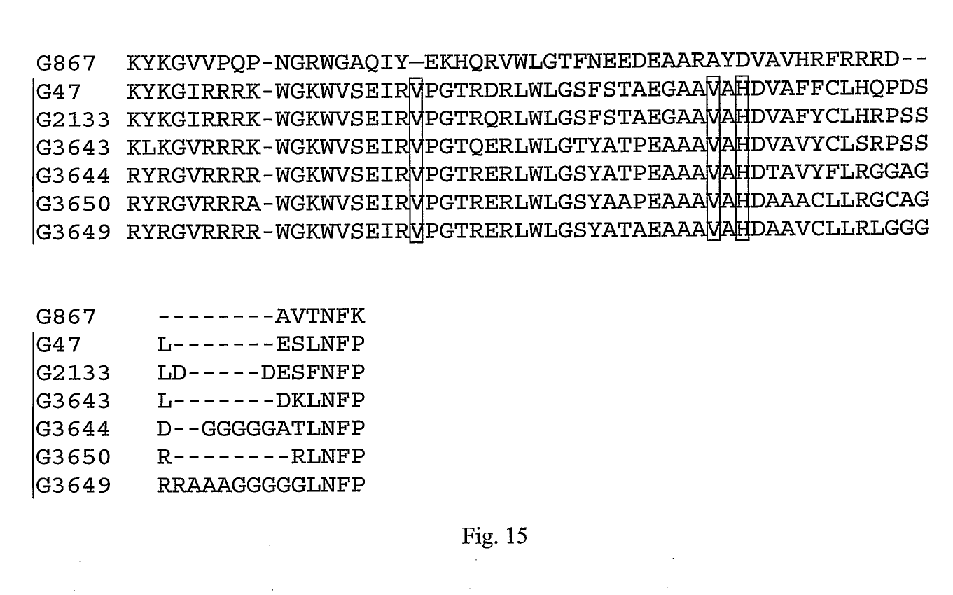

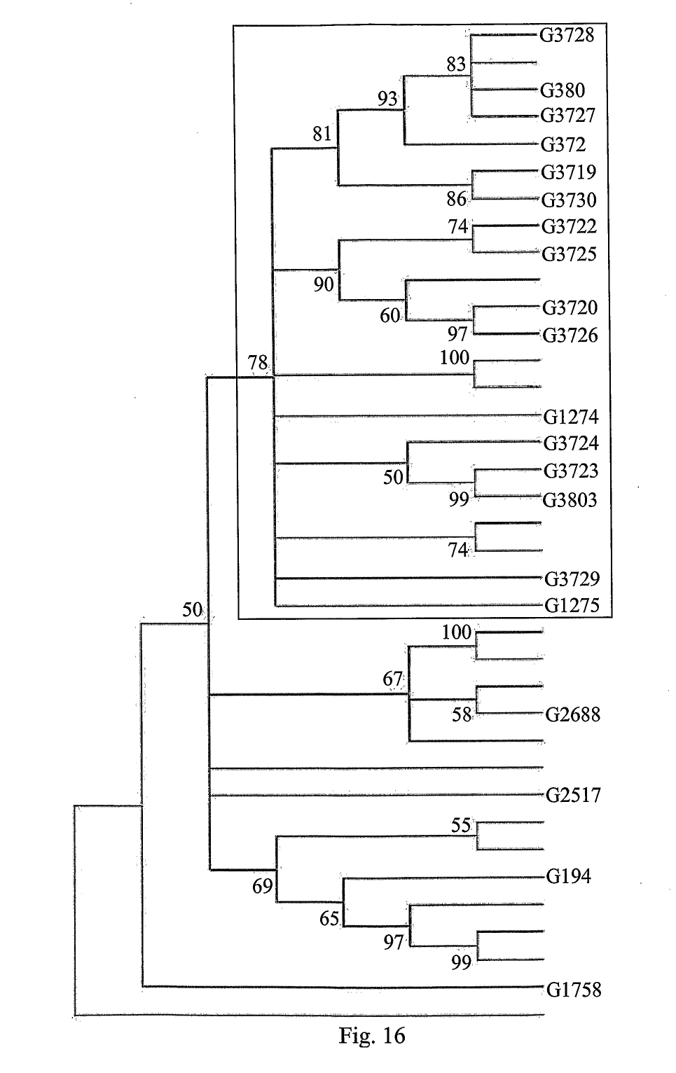

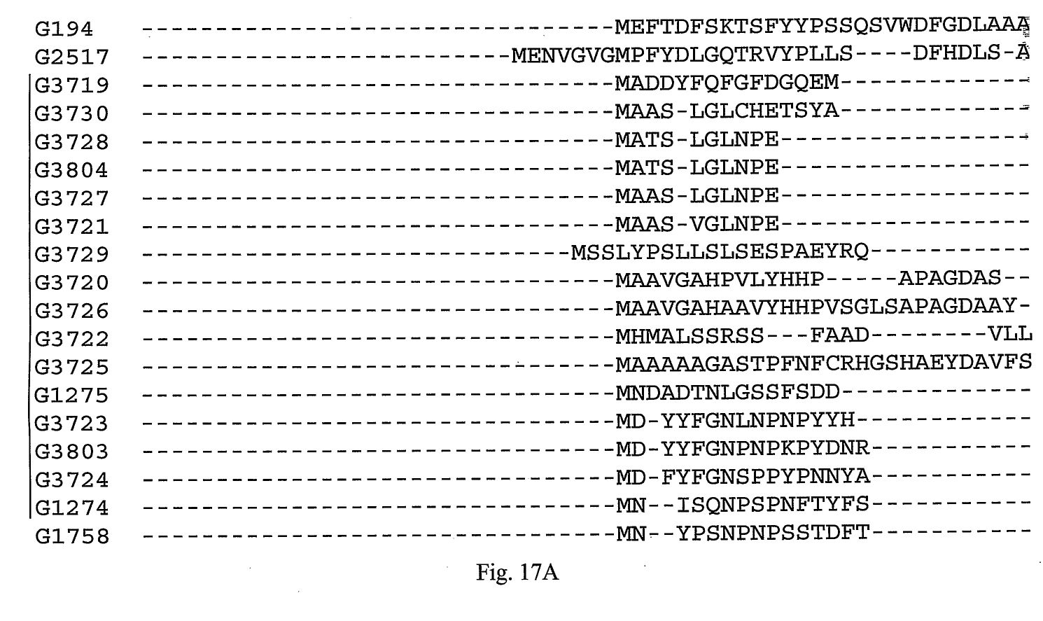

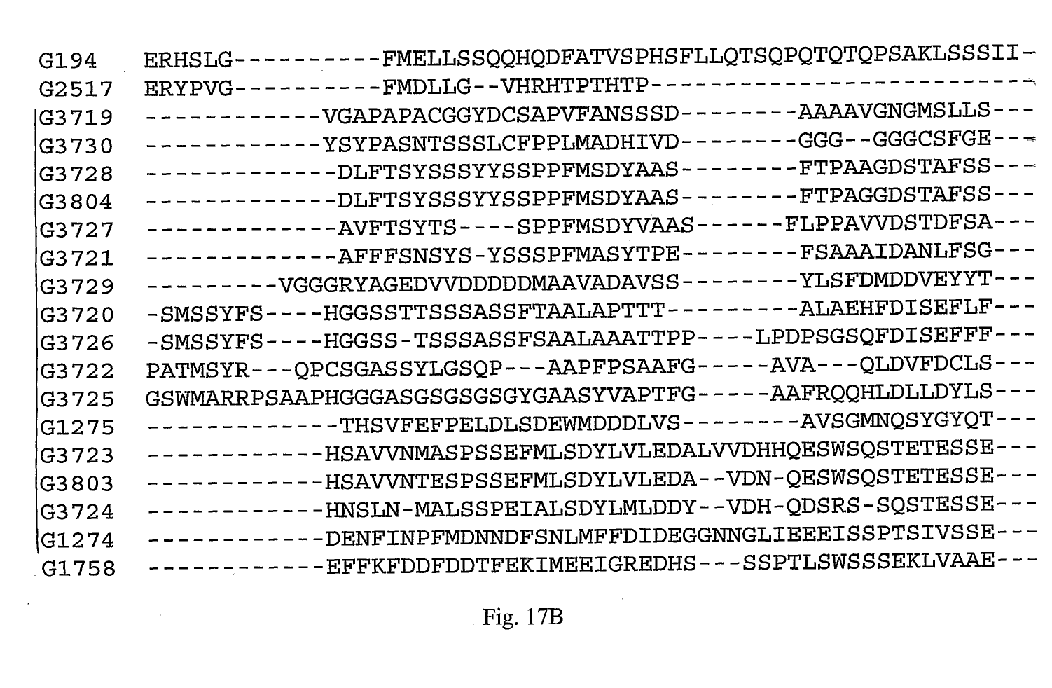

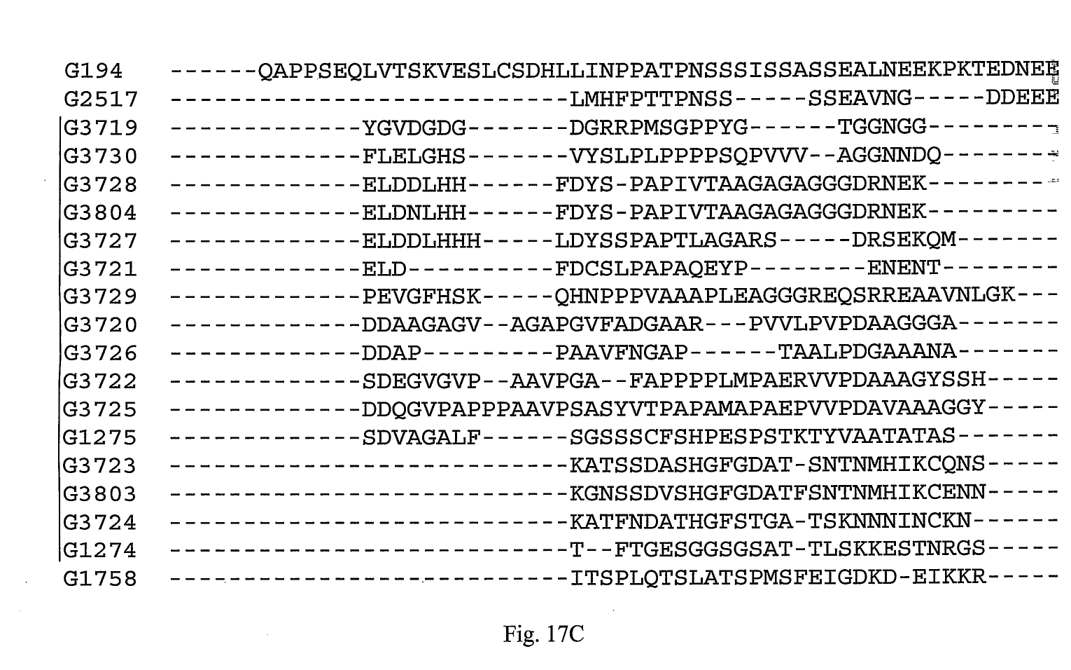

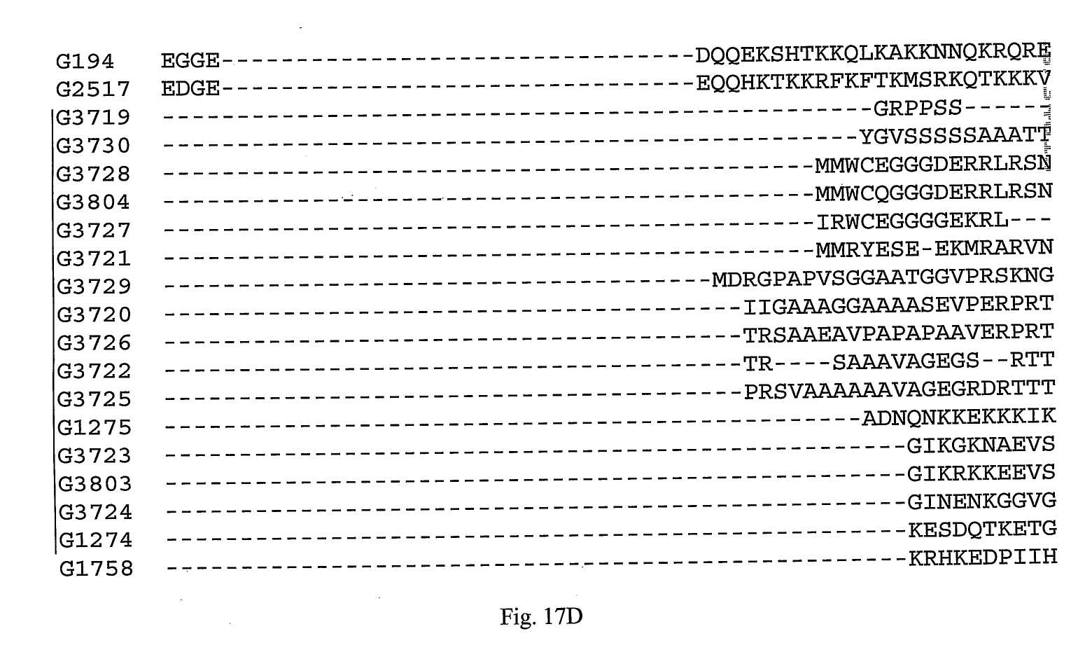

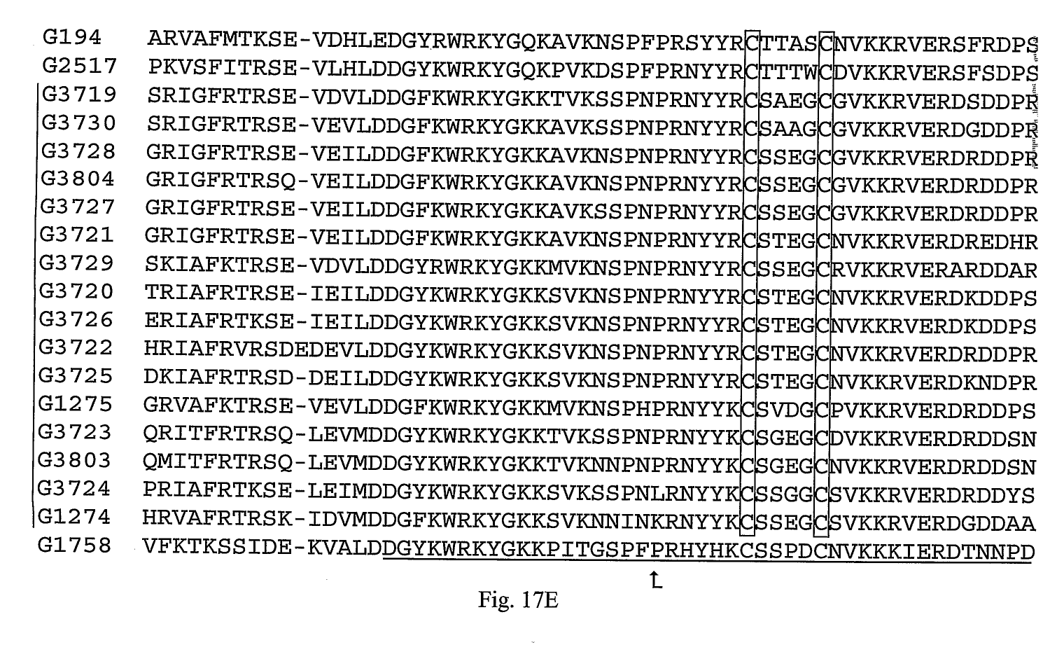

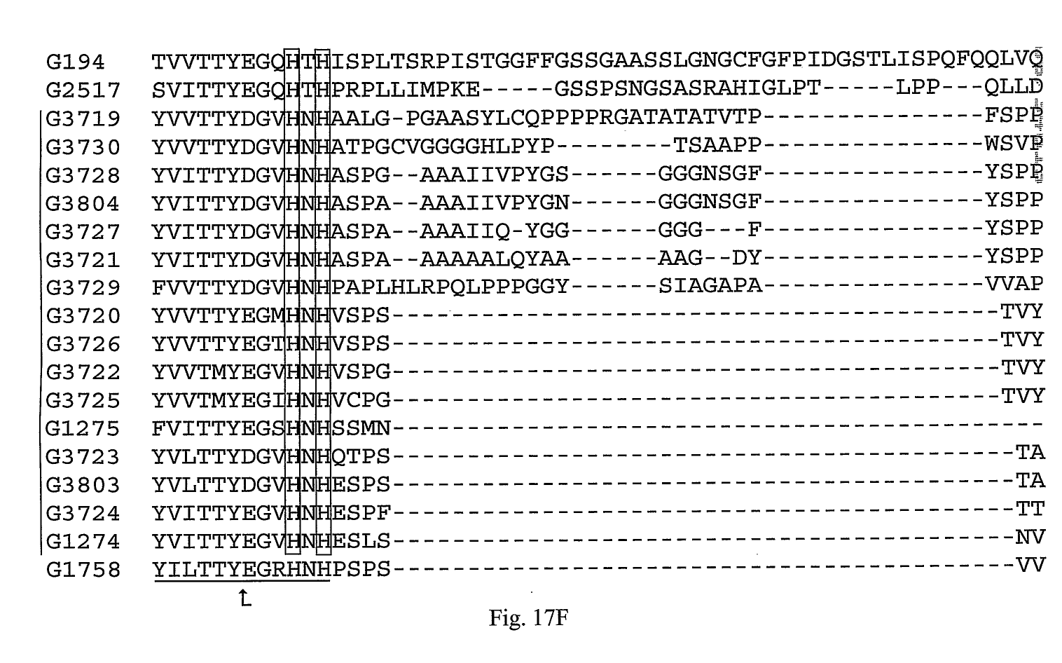

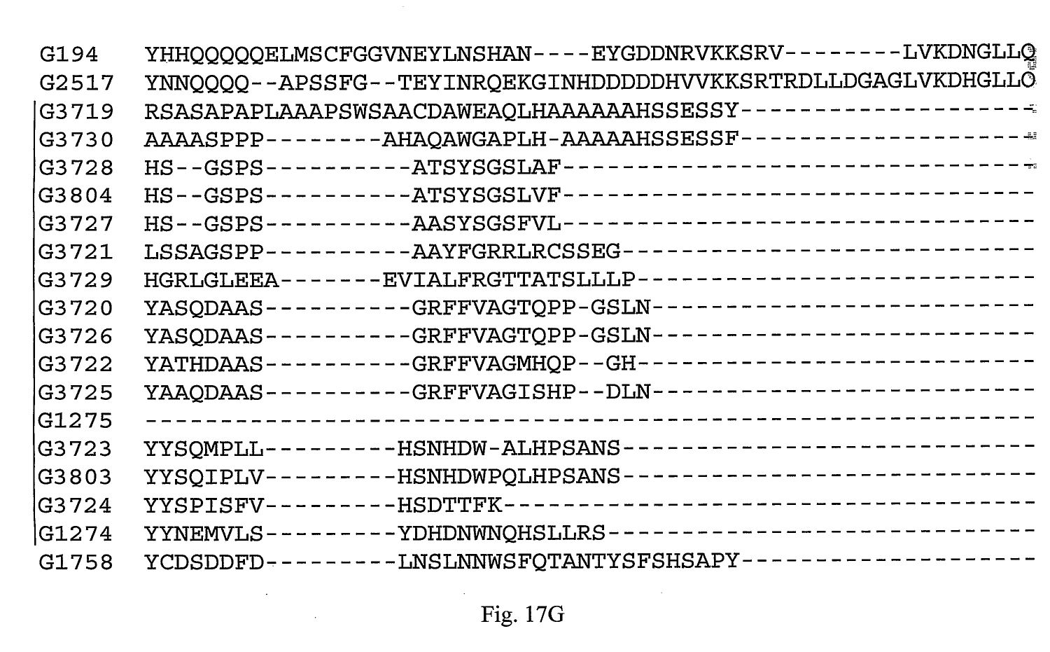

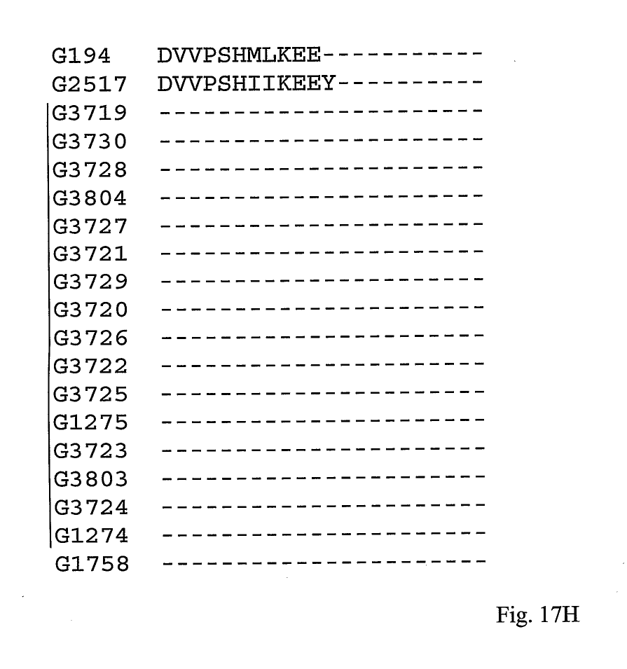

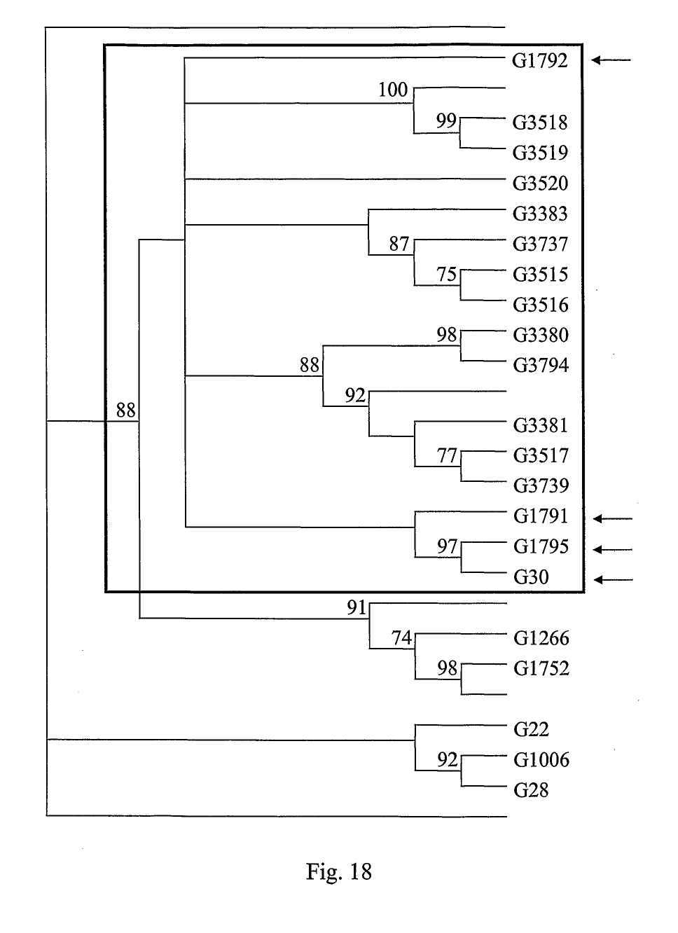

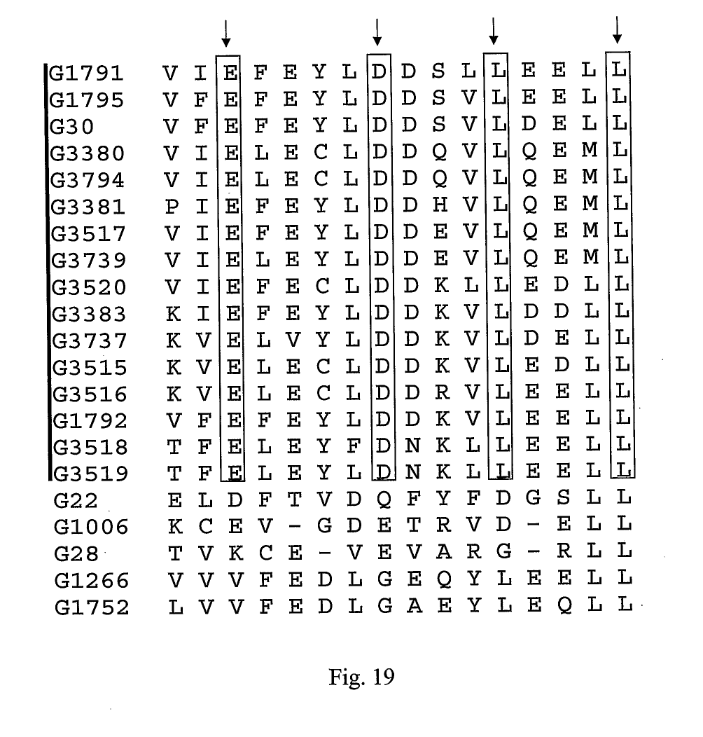

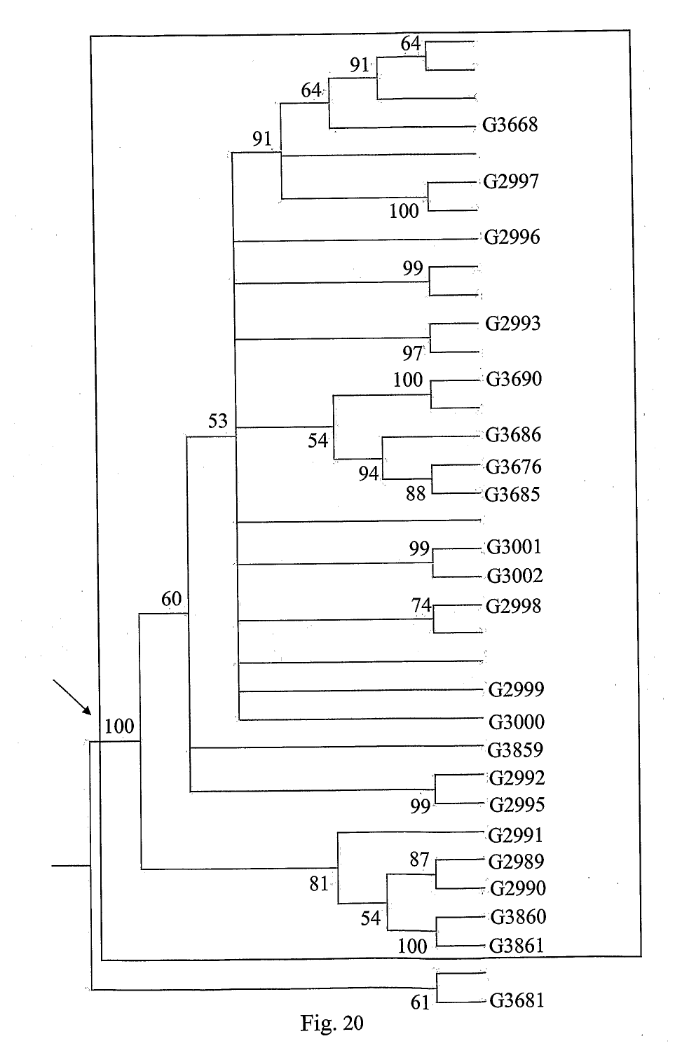

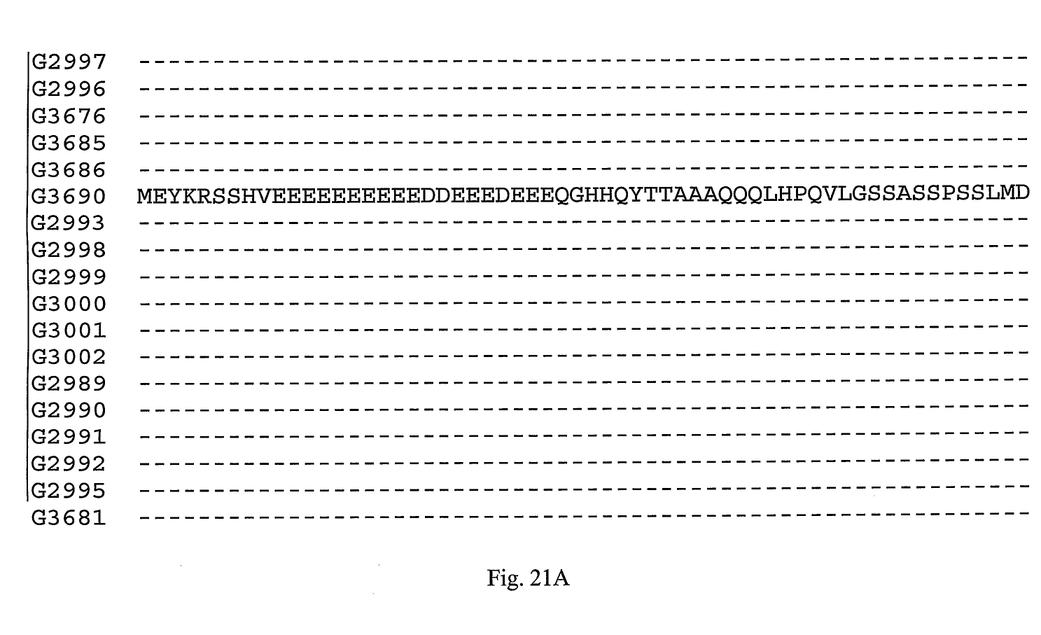

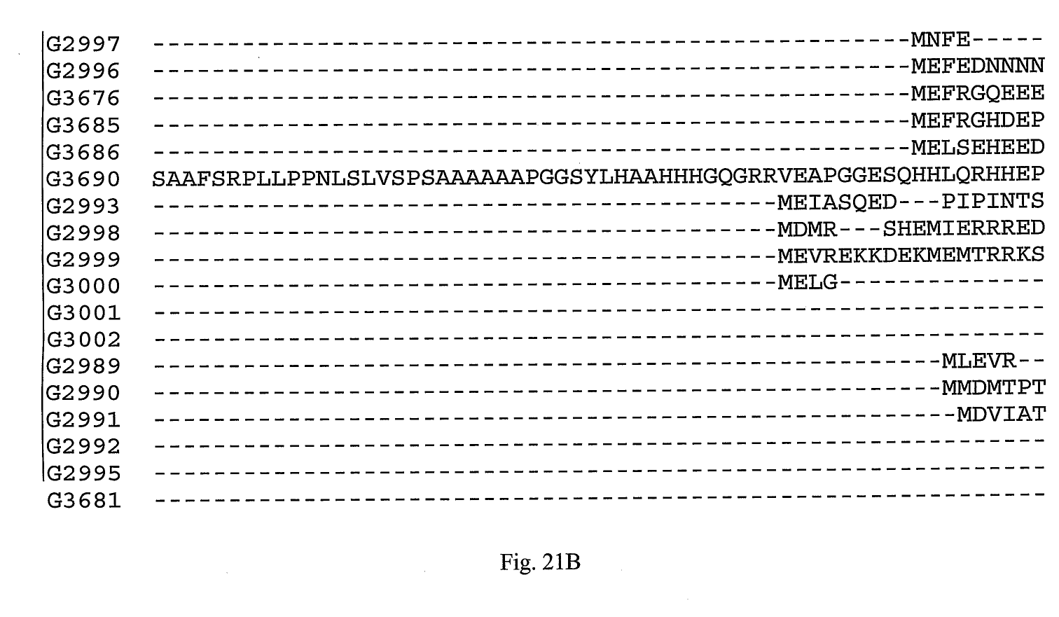

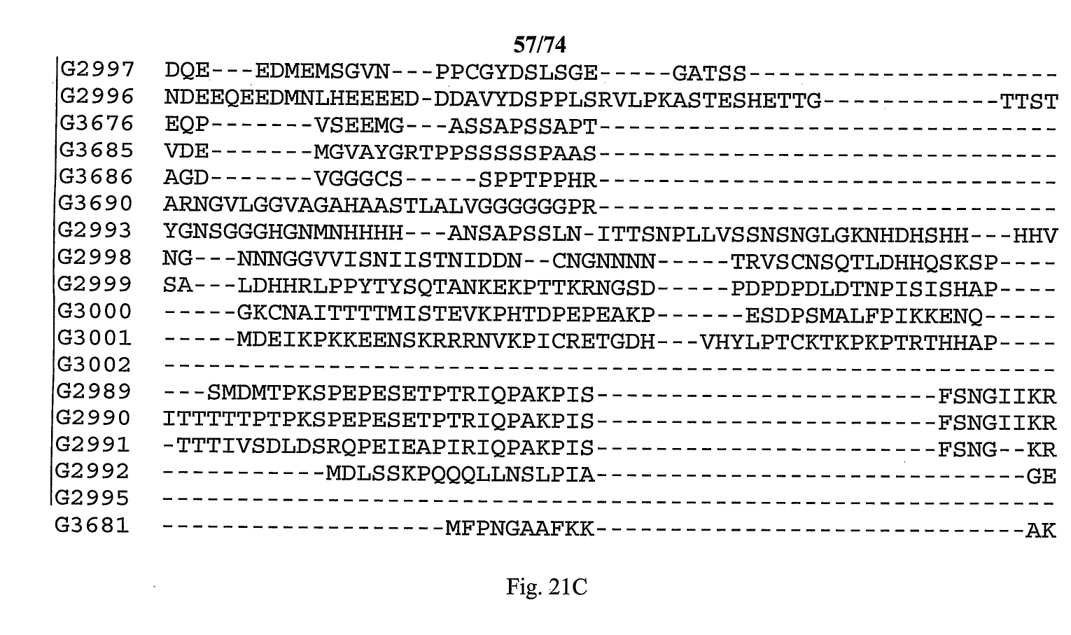

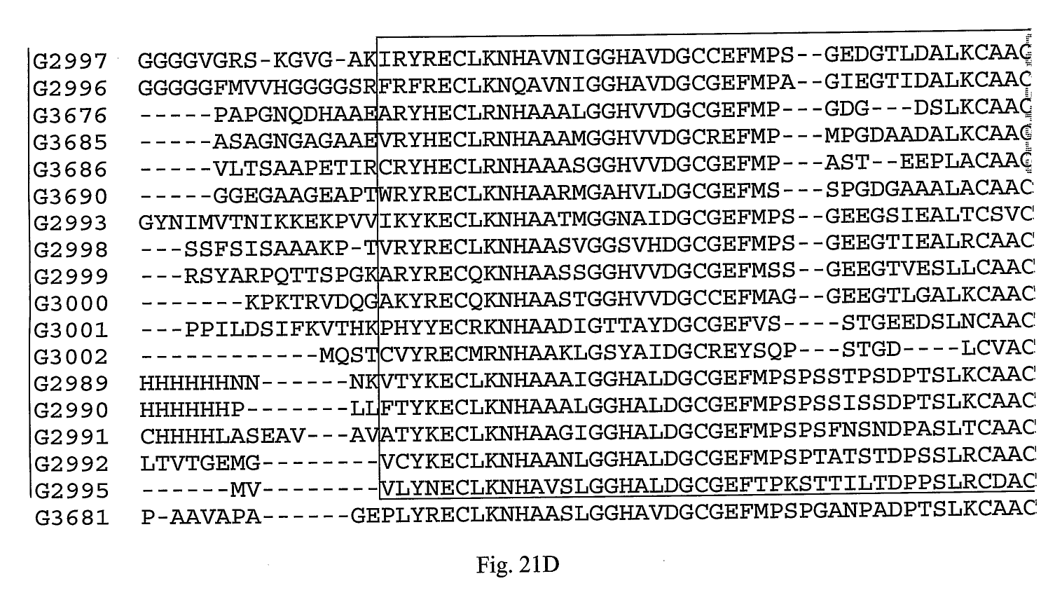

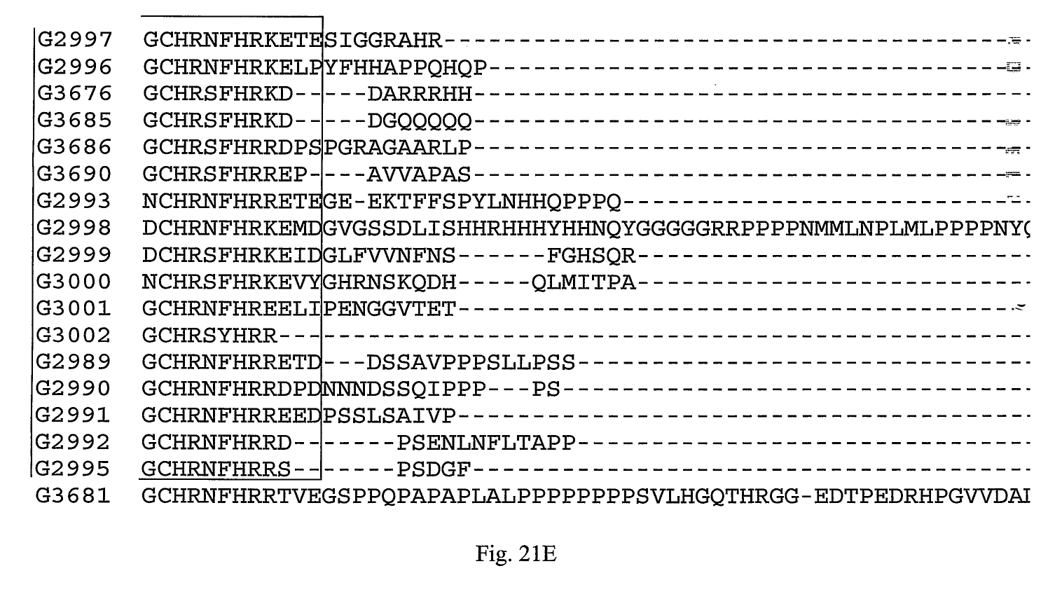

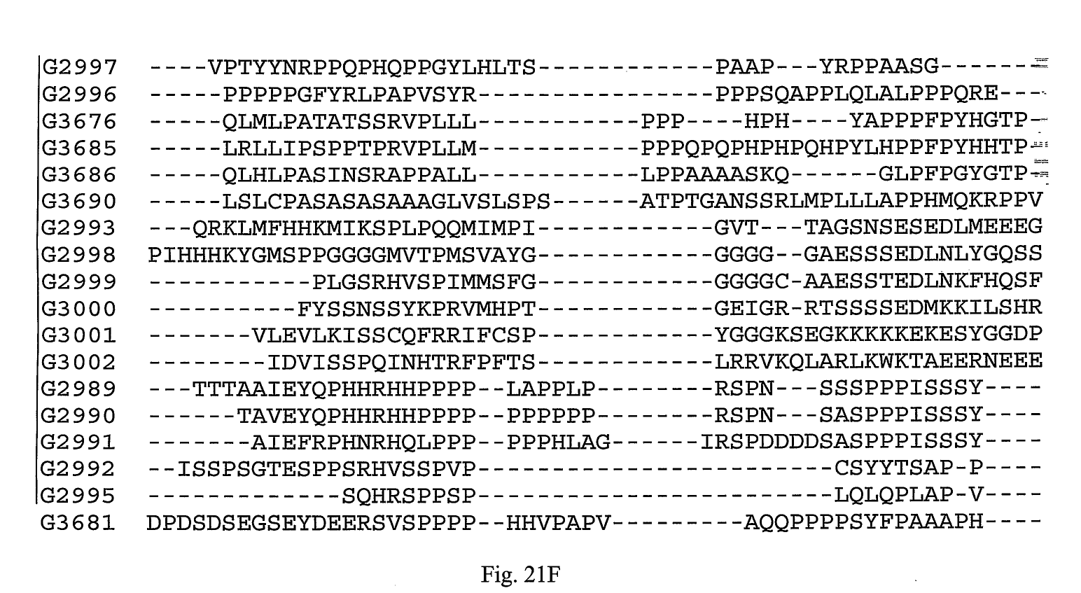

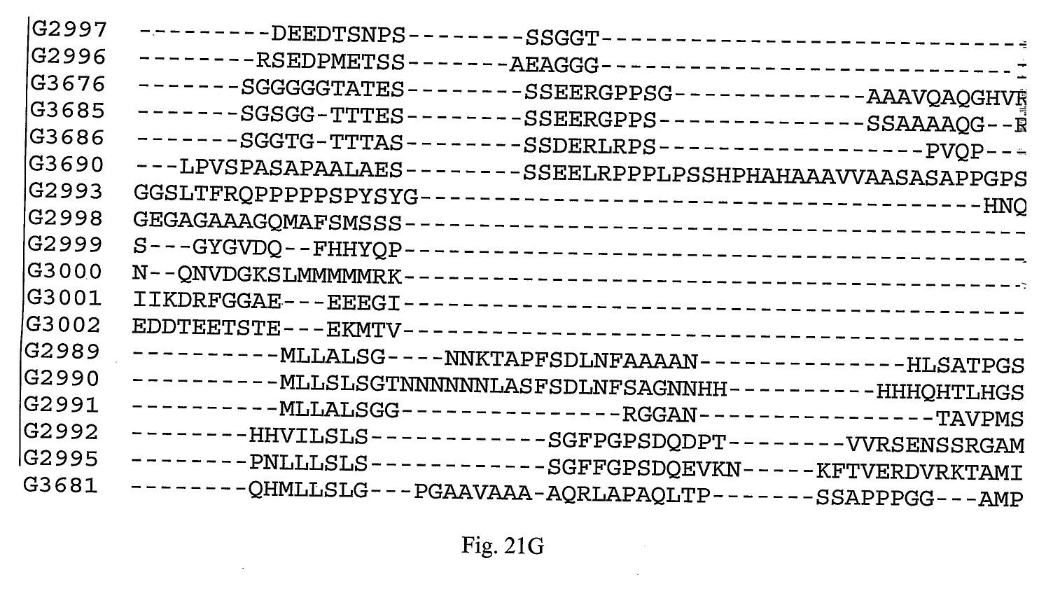

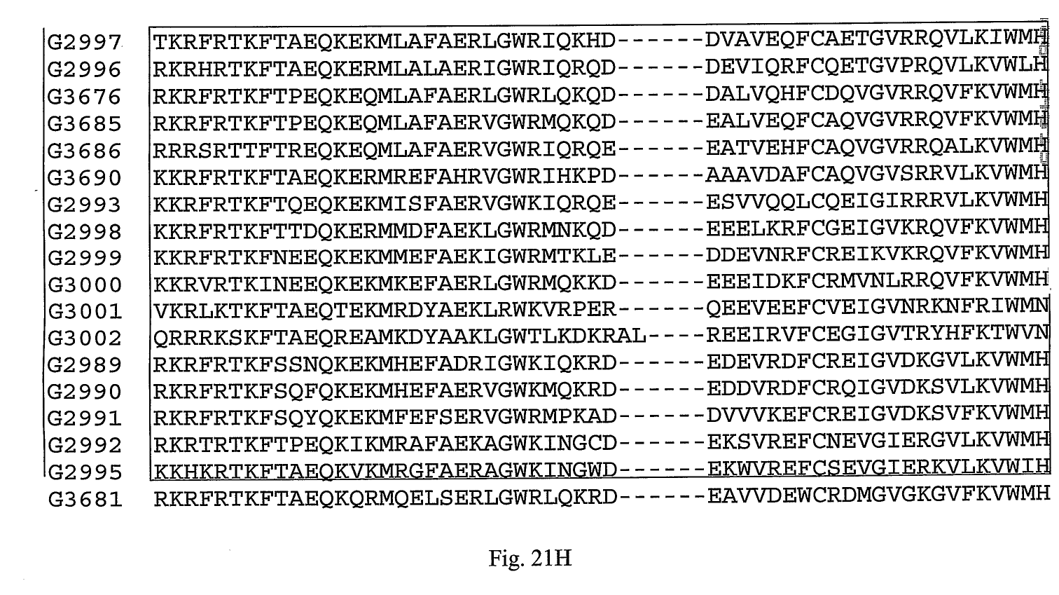

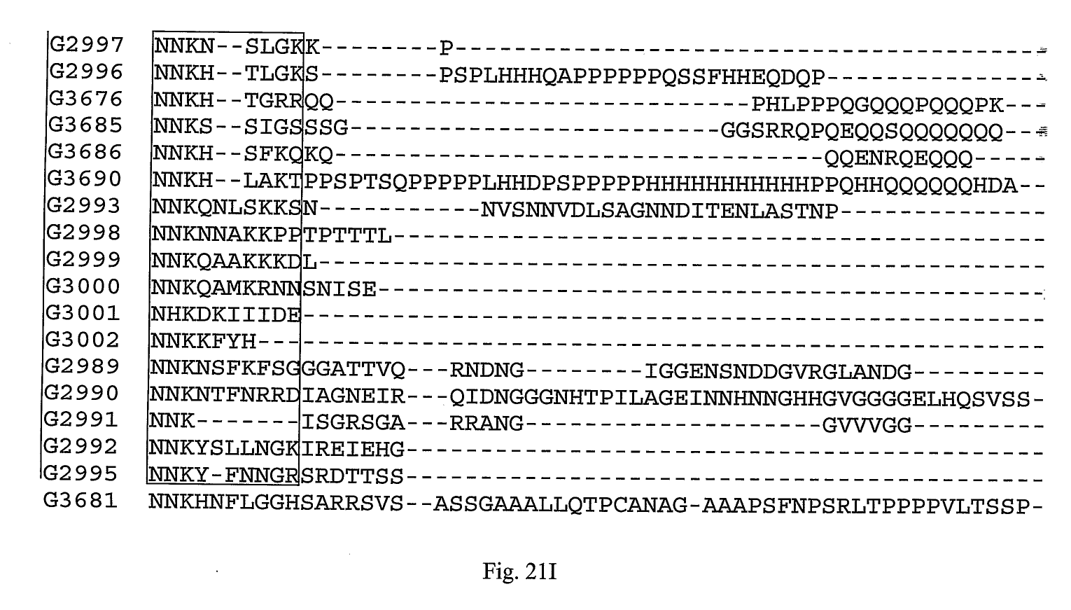

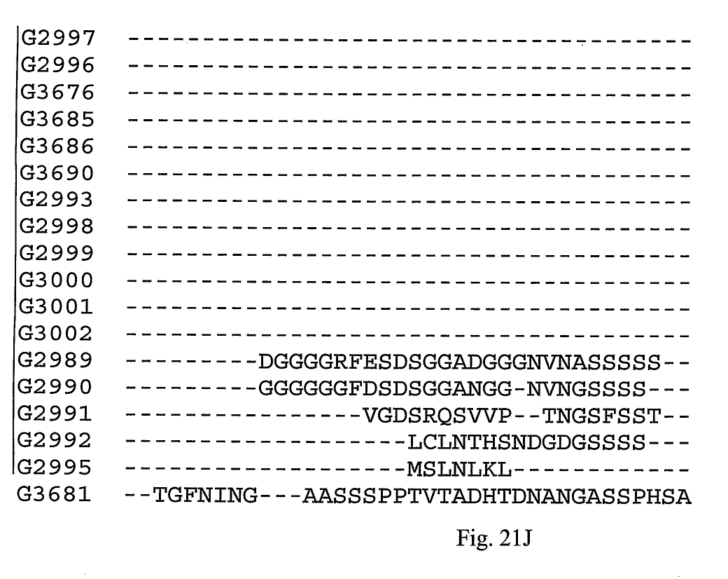

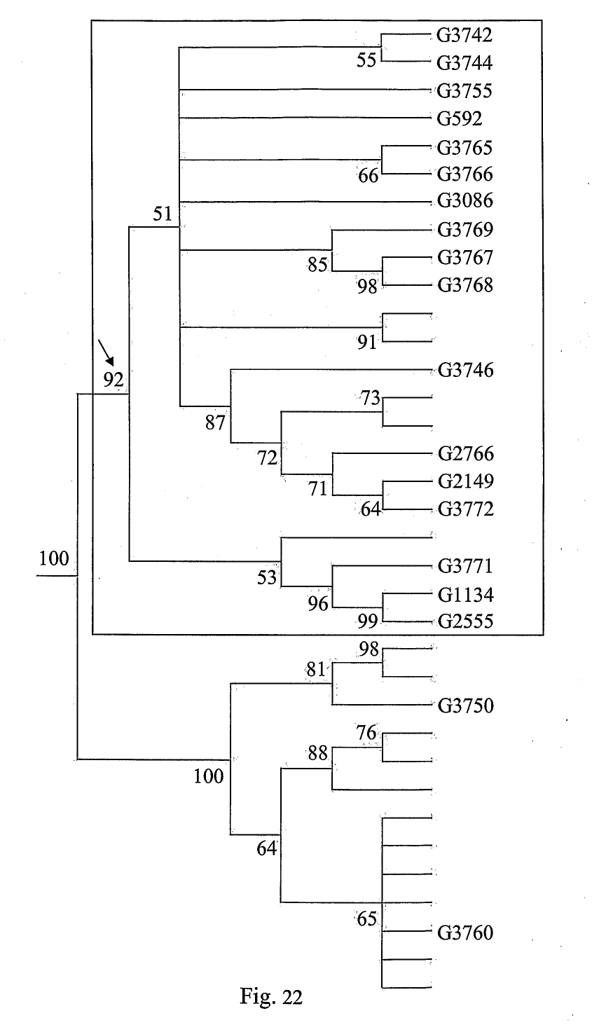

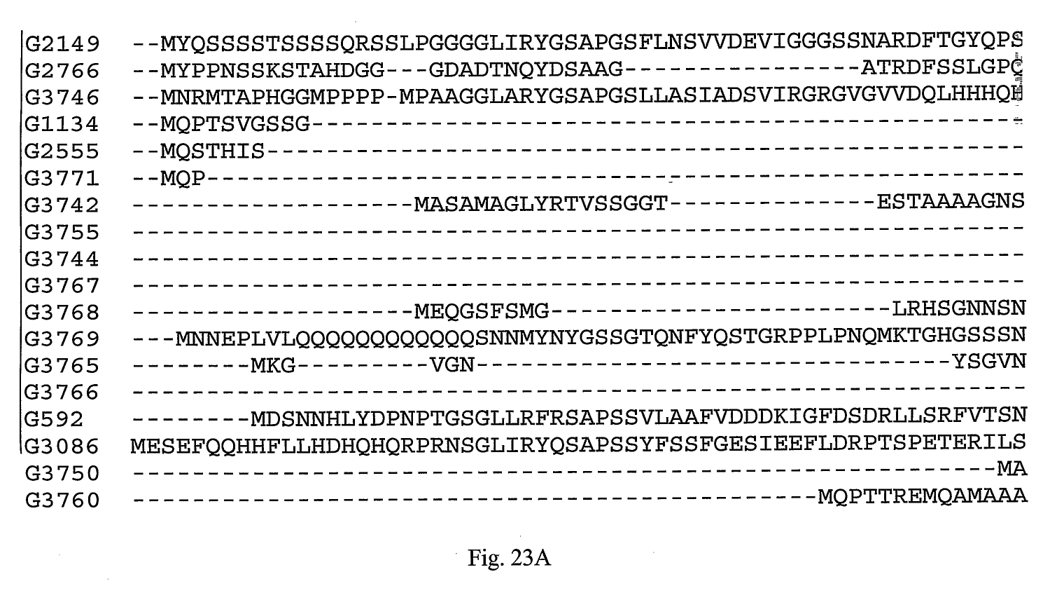

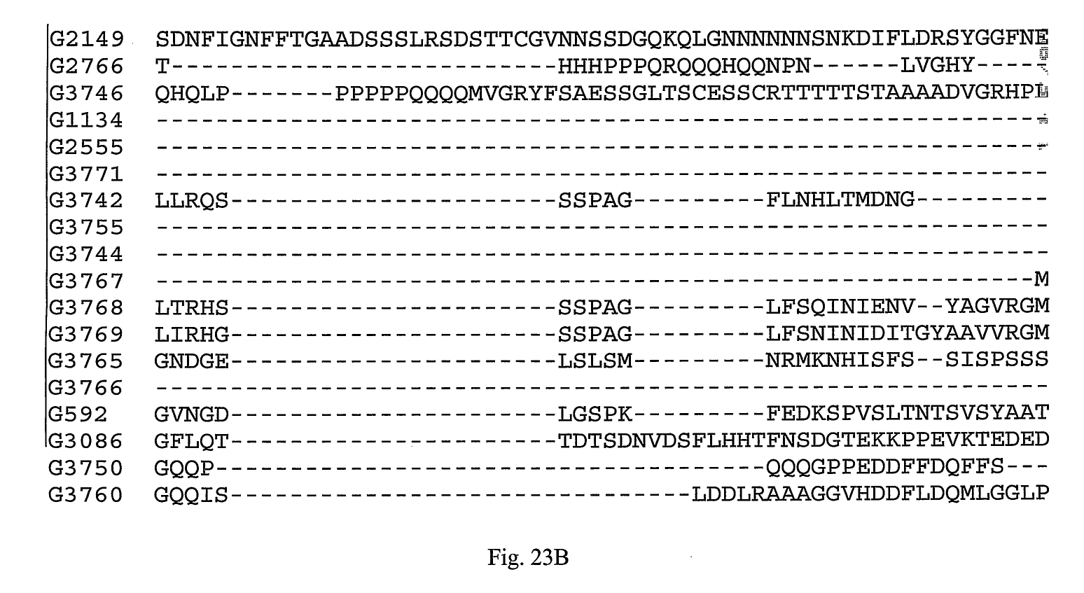

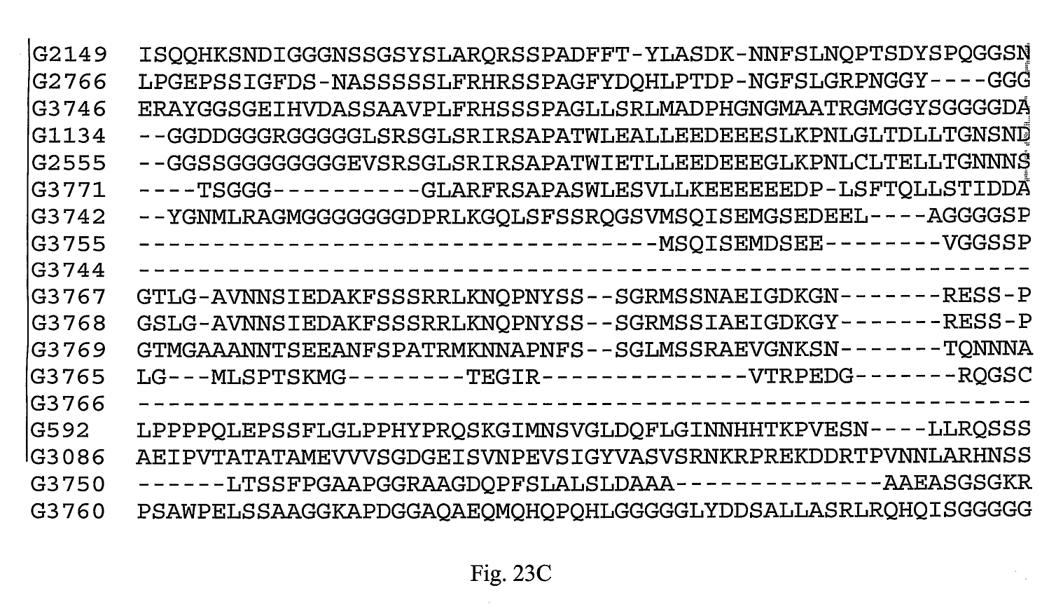

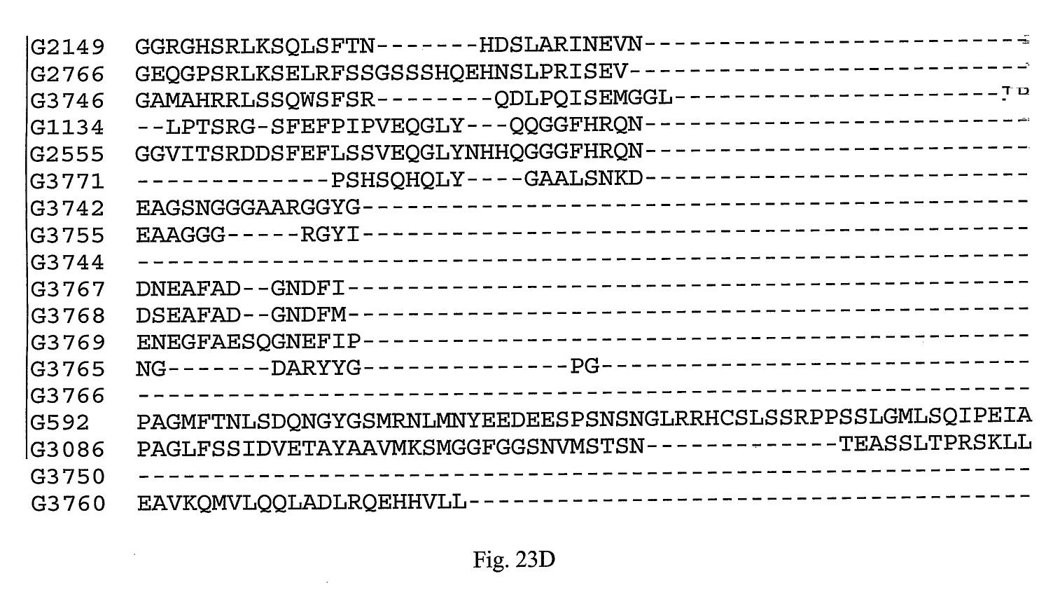

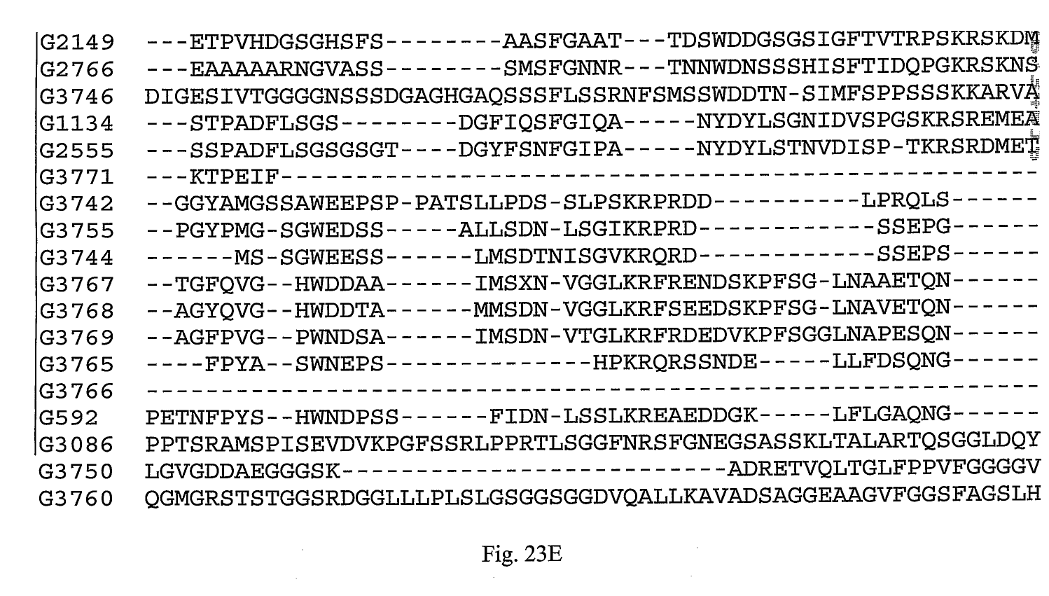

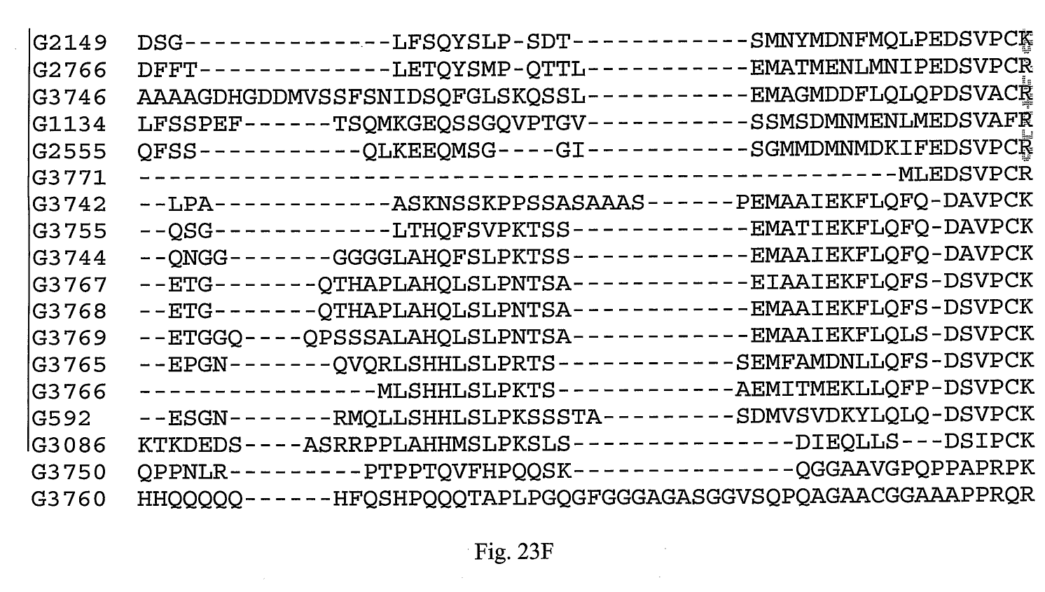

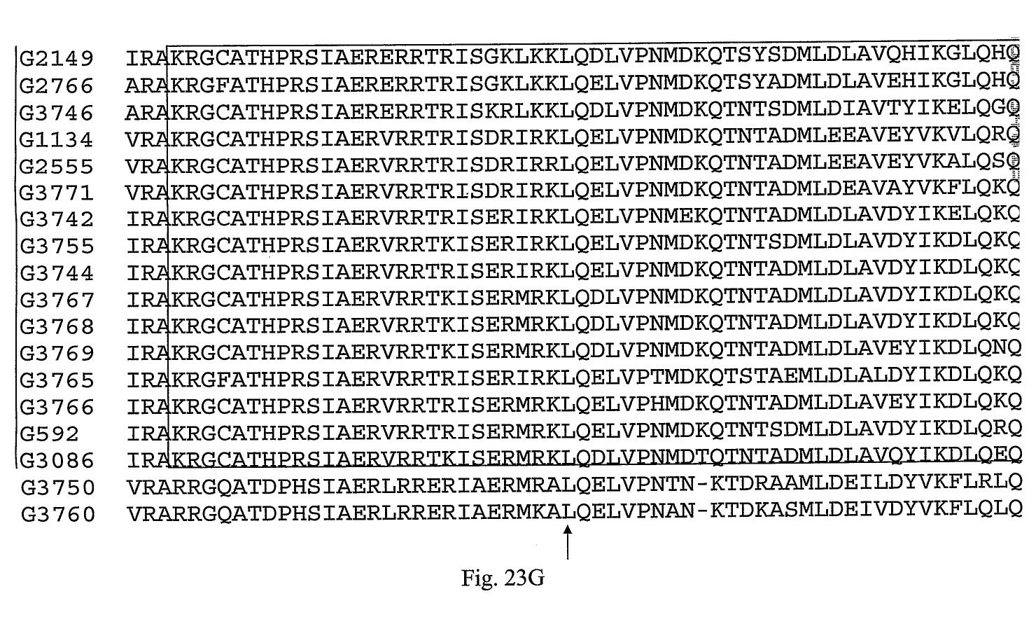

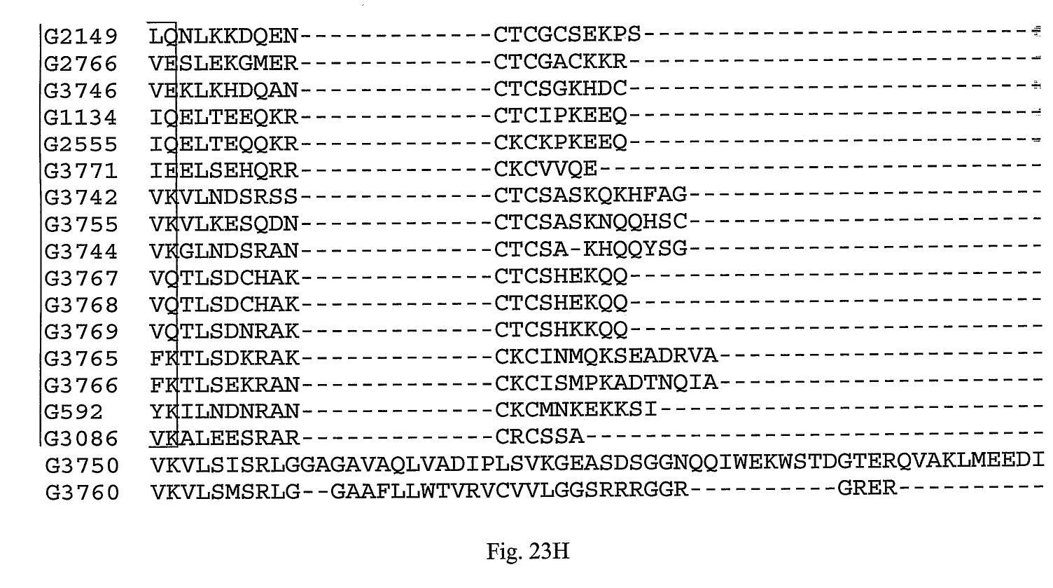

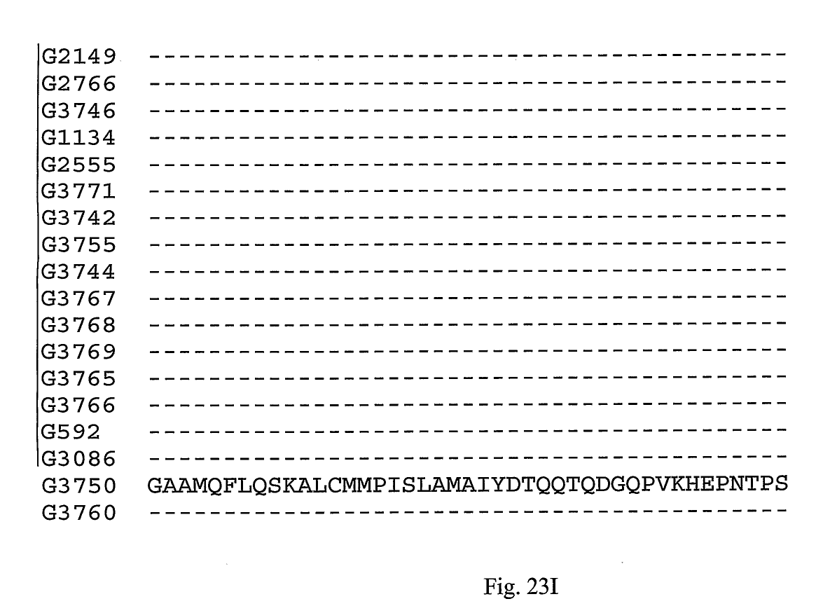

FIG. 12 shows a phylogenetic analysis of G28 and closely related sequences. A phylogenetic tree and multiple sequence alignments of G28 and related fall length proteins were constructed using ClustalW (CLUSTAL W Multiple Sequence Alignment Program version 1.83, 2003) and MEGA2 (http://www.megasoftware.net) software with the multiple alignment parameters the same as for the G1073 tree described above for FIG. 9. A FastA formatted alignment was then used to generate a phylogenetic tree in MEGA2 using the neighbor joining algorithm and a p-distance model. A test of phylogeny was done via bootstrap with 1000 replications and Random Seed set to default. Cut off values of the bootstrap tree were set to 50%. Closely-related sequences to G28 are considered as being those polypeptides within the node of the tree (the arrow indicates this node identifying an ancestral sequence, from which sequences with related functions to G28 were descended) below with a bootstrap value of 99, bounded in this tree by G3717 and G22. Sequences shown in this tree include G3717 (SEQ ID NO: 154), G3718 (SEQ ID NO: 156), G28 (SEQ ID NO: 148), G3659 (SEQ ID NO: 150), G1006 (SEQ ID NO: 152), G3660 (SEQ ID NO: 158), G3661 (SEQ ID NO: 162), G3848 (SEQ ID NO: 160), G3856 (SEQ ID NO: 166), G3430 (SEQ ID NO: 168), G3864 (SEQ ID NO: 164), G3841 (SEQ ID NO: 170), and G22 (SEQ ID NO: 172).

FIGS. 13A-13G are a Clustal W multiple sequence alignment of G28 and related proteins (CLUSTAL W Multiple Sequence Alignment Program version 1.83, 2003). The vertical lines in each of FIGS. 13A-13G indicate members of the G28 clade. The box spanning 13D-13E indicates the AP2 domain of the sequences within the clade. Sequences shown in this alignment include G1006 (SEQ ID NO: 152), G3660 (SEQ ID NO: 158), G28 (SEQ ID NO: 148), G3659 (SEQ ID NO: 150), G3717 (SEQ ID NO: 154), G3718 (SEQ ID NO: 156), G3430 (SEQ ID NO: 168), G3864 (SEQ ID NO: 164), G3856 (SEQ ID NO: 166), G3661 (SEQ ID NO: 162), G3848 (SEQ ID NO: 160), G3841 (SEQ ID NO: 170), G22 (SEQ ID NO: 172), G1752 (SEQ ID NO: 402), G1266 (SEQ ID NO: 254), G1795 (SEQ ID NO: 224), G30 (SEQ ID NO: 226), G1791 (SEQ ID NO: 230), and G1792 (SEQ ID NO: 222).

In FIG. 14, A phylogenetic tree and multiple sequence alignments of G47 and related fall length proteins were constructed using ClustalW (CLUSTAL W Multiple Sequence Alignment Program version 1.83, 2003) and MEGA2 (http://www.megasoftware.net) software. ClustalW multiple alignment parameters were the same as described above for G1073, FIG. 9. A FastA formatted alignment was then used to generate a phylogenetic tree in MEGA2 using the neighbor joining algorithm and a p-distance model. A test of phylogeny was done via bootstrap with 1000 replications and Random Seed set to default. Cut off values of the bootstrap tree were set to 50%. Members of the G47 clade are represented by the proteins in the large box and within the node of the tree below with a bootstrap value of 93, bounded by G3644 and G47, as indicated by the sequences within the box. Sequences shown in this tree include G2115 (SEQ ID NO: 406), G3644 (SEQ ID NO: 182), G3650 (SEQ ID NO: 180), G3649 (SEQ ID NO: 184), G3643 (SEQ ID NO: 178), G2133 (SEQ ID NO: 176), G47 (SEQ ID NO: 174), and G867 (SEQ ID NO: 88).

FIG. 15 shows a Clustal W alignment of the AP2 domains of the G47 clade. The three residues indicated by the boxes define the G47 clade; clade members (indicated by the vertical line at left) have two valines and a histidine residue at these positions, respectively. In the sequences examined to date, the AP2 domain of G47 clade members comprises VX₁₉VAHD, where X is any amino acid residue. The “VAHD subsequence” consisting of the amino acid residues V-A-H-D is a combination not found in other Arabidopsis AP2/ERF proteins. Sequences appearing in this alignment include G867 (SEQ ID NO: 88), and G47 clade members G47 (SEQ ID NO: 174), G2133 (SEQ ID NO: 176), G3643 (SEQ ID NO: 178), G3644 (SEQ ID NO: 182), G3650 (SEQ ID NO: 180), and G3649 (SEQ ID NO: 184).

In FIG. 16, A phylogenetic tree and multiple sequence alignments of G1274 and related full length proteins were constructed using ClustalW (CLUSTAL W Multiple Sequence Alignment Program version 1.83, 2003) and MEGA2 (http://www.megasoftware.net) software. ClustalW multiple alignment parameters were the same as described above for G1073, FIG. 9. FastA formatted alignment was then used to generate a phylogenetic tree in MEGA2 using the neighbor joining algorithm and a p-distance model. A test of phylogeny was done via bootstrap with 1000 replications and Random Seed set to default. Cut off values of the bootstrap tree were set to 50%. Members of the G1274 clade are represented by the proteins in the large box and within the node of the tree below with a bootstrap value of 78, bounded by G3728 and G1275. Sequences shown in this tree include G3728 (SEQ ID NO: 190), G3804 (SEQ ID NO: 192), G3727 (SEQ ID NO: 196), G3721 (SEQ ID NO: 198), G3719 (SEQ ID NO: 212), G3730 (SEQ ID NO: 210), G3722 (SEQ ID NO: 200), G3725 (SEQ ID NO: 214), G3720 (SEQ ID NO: 204), G3726 (SEQ ID NO: 202), G1274 (SEQ ID NO: 186), G3724 (SEQ ID NO: 188), G3723 (SEQ ID NO: 206), G3803 (SEQ ID NO: 194), G3729 (SEQ ID NO: 216), G1275 (SEQ ID NO: 208), G2688 (SEQ ID NO: 398), G2517 (SEQ ID NO: 220), G194 (SEQ ID NO: 218), and G1758 (SEQ ID NO: 394).

FIGS. 17A-17H represent a Clustal W alignment of the G1274 clade and related proteins. The vertical line at left indicates G1274 clade members. The “WRKY” (DNA binding) domain, indicated by the right-angled arrow “ [symbol] ” and the line that spans FIGS. 17E-17F, and zinc finger motif (with the pattern of potential zinc ligands C-X₄₋₅-C-X₂₂₋₂₃-H-X₁-H) are also shown (the potential zinc ligands appear in boxes in FIGS. 17E-17F). Sequences in this tree include G194 (SEQ ID NO: 218), G2517 (SEQ ID NO: 220), G3719 (SEQ ID NO: 212), G3730 (SEQ ID NO: 210), G3728 (SEQ ID NO: 190), G3804 (SEQ ID NO: 192), G3727 (SEQ ID NO: 196), G3721 (SEQ ID NO: 198), G3729 (SEQ ID NO: 216), G3720 (SEQ ID NO: 204), G3726 (SEQ ID NO: 202), G3722 (SEQ ID NO: 200), G3725 (SEQ ID NO: 214), G1275 (SEQ ID NO: 208), G3723 (SEQ ID NO: 206), G3803 (SEQ ID NO: 194), G3724 (SEQ ID NO: 188), G1274 (SEQ ID NO: 186), and G1758 (SEQ ID NO: 394).

FIG. 18 is a Clustal W-generated phylogenetic tree created using the conserved AP2 domain and EDLL domain of G1792-related paralogs and orthologs. Members of the G1792 clade are found within the large box. Arabidopsis paralogs are designated by arrows. Sequences shown in this tree include G1792 (SEQ ID NO: 22), G3518 (SEQ ID NO: 246), G3519 (SEQ ID NO: 232), G3520 (SEQ ID NO: 242), G3383 (SEQ ID NO: 228), G3737 (SEQ ID NO: 236), G3515 (SEQ ID NO: 238), G3516 (SEQ ID NO: 240), G3380 (SEQ ID NO: 250), G3794 (SEQ ID NO: 252), G3381 (SEQ ID NO: 234), G3517 (SEQ ID NO: 244), G3739 (SEQ ID NO: 248), G1791 (SEQ ID NO: 230), G1795 (SEQ ID NO: 224), G30 (SEQ ID NO: 226), G1266 (SEQ ID NO: 254), G1752 (SEQ ID NO: 402), G22 (SEQ ID NO: 172), G1006 (SEQ ID NO: 152), and G28 (SEQ ID NO: 148).

FIG. 19 shows an alignment of a portion of the G1792 activation domain designated the EDLL domain, a novel conserved domain for the G1792 clade. All clade members (in this figure the clade members are indicated by the vertical line to the left of the alignment) contain a glutamic acid residue at position 3, an aspartic acid residue at position 8, and leucine residues at positions 12 and 16 of the domain (thus comprising the subsequence EX₄DX₃LX₃L, where X is any amino acid residue), said residues indicated by the arrows above the alignment. Sequences shown in this alignment include G1791 (SEQ ID NO: 230), G1795 (SEQ ID NO: 224), G30 (SEQ ID NO: 226), G3380 (SEQ ID NO: 250), G3794 (SEQ ID NO: 252), G3381 (SEQ ID NO: 234), G3517 (SEQ ID NO: 244), G3739 (SEQ ID NO: 248), G3520 (SEQ ID NO: 242), G3383 (SEQ ID NO: 228), G3737 (SEQ ID NO: 236), G3515 (SEQ ID NO: 238), G3516 (SEQ ID NO: 240), G1792 (SEQ ID NO: 22), G3518 (SEQ ID NO: 246), G3519 (SEQ ID NO: 232), G22 (SEQ ID NO: 172), G1006 (SEQ ID NO: 152), G28 (SEQ ID NO: 148), G1266 (SEQ ID NO: 254), and G1752 (SEQ ID NO: 402).

FIG. 20 is a phylogenetic tree of G2999 and related proteins constructed using ClustalW and MEGA2 (http://www.megasoftware.net) software. ClustalW multiple alignment parameters used were the same as described for FIG. 9, above. A FastA formatted alignment was then used to generate a phylogenetic tree in MEGA2 using the neighbor joining algorithm and a p-distance model. A test of phylogeny was done via bootstrap with 1000 replications and Random Seed set to default. Cut off values of the bootstrap tree were set to 50%. The arrow indicates the strong node indicating the common ancestor of the G2999 clade (sequences in box). Sequences shown in this tree include G3668 (SEQ ID NO: 416), G2997 (SEQ ID NO: 264), G2996 (SEQ ID NO: 270), G2993 (SEQ ID NO: 276), G3690 (SEQ ID NO: 262), G3686 (SEQ ID NO: 268), G3676 (SEQ ID NO: 266), G3685 (SEQ ID NO: 274), G3001 (SEQ ID NO: 272), G3002 (SEQ ID NO: 290), G2998 (SEQ ID NO: 258), G2999 (SEQ ID NO: 256), G3000 (SEQ ID NO: 260), G3859 (SEQ ID NO: 414), G2992 (SEQ ID NO: 286), G2995 (SEQ ID NO: 288), G2991 (SEQ ID NO: 282), G2989 (SEQ ID NO: 280), G2990 (SEQ ID NO: 284), G3860 (SEQ ID NO: 418), G3861 (SEQ ID NO: 420), and G3681 (SEQ ID NO: 278).

FIGS. 21A-21J are a Clustal W-generated multiple sequence alignment of G2999 and related sequences. The vertical line identifies members of the G2999 clade. The box spanning FIGS. 21D-21E indicates the ZF domains of the sequences within the clade. The box spanning FIGS. 21H-21I indicates the HD domains of the sequences in the G2999 clade. Sequences shown in this alignment include G2997 (SEQ ID NO: 264), G2996 (SEQ ID NO: 270), G3676 (SEQ ID NO: 266), G3685 (SEQ ID NO: 274), G3686 (SEQ ID NO: 268), G3690 (SEQ ID NO: 262), G2993 (SEQ ID NO: 276), G2998 (SEQ ID NO: 258), G2999 (SEQ ID NO: 256), G3000 (SEQ ID NO: 260), G3001 (SEQ ID NO: 272), G3002 (SEQ ID NO: 290), G2989 (SEQ ID NO: 280), G2990 (SEQ ID NO: 284), G2991 (SEQ ID NO: 282), G2992 (SEQ ID NO: 286), G2995 (SEQ ID NO: 288), and G3681 (SEQ ID NO: 278).

FIG. 22 is a phylogenetic tree of G3086 and related fall length proteins, constructed using MEGA2 (http://www.megasoftware.net) software. A FastA formatted alignment was used to generate a phylogenetic tree in MEGA2 using the neighbor joining algorithm and a p-distance model. A test of phylogeny was done via bootstrap with 1000 replications and Random Seed set to default. Cut off values of the bootstrap tree were set to 50%. Orthologs of G3086 are considered as being those proteins within the node of the tree below with a bootstrap value of 92 (arrow), bounded by G3742 and G2555 (indicated by the large box). Sequences shown in this tree include G3742 (SEQ ID NO: 308), G3744 (SEQ ID NO: 300), G3755 (SEQ ID NO: 302), G592 (SEQ ID NO: 306), G3765 (SEQ ID NO: 314), G3766 (SEQ ID NO: 304), G3086 (SEQ ID NO: 292), G3769 (SEQ ID NO: 296), G3767 (SEQ ID NO: 298), G3768 (SEQ ID NO: 294), G3746 (SEQ ID NO: 310), G2766 (SEQ ID NO: 322), G2149 (SEQ ID NO: 320), G3772 (SEQ ID NO: 200), G3771 (SEQ ID NO: 312), G1134 (SEQ ID NO: 316), G2555 (SEQ ID NO: 318), G3750 (SEQ ID NO: 326), and G3760 (SEQ ID NO: 324).

FIGS. 23A-231 represent a Clustal W-generated multiple sequence alignment of G3086 and related sequences. The vertical line to the left of the alignment on each page identifies members of the G3086 clade. The box spanning FIGS. 23G-23H indicates a conserved domain found within the clade member sequences. An invariant leucine residue found in all bHLH proteins, indicated by the arrow in FIG. 23G, is required for protein dimerization. Sequences shown in this alignment include G2149 (SEQ ID NO: 320), G2766 (SEQ ID NO: 322), G3746 (SEQ ID NO: 310), G1134 (SEQ ID NO: 316), G2555 (SEQ ID NO: 318), G3771 (SEQ ID NO: 312), G3742 (SEQ ID NO: 308), G3755 (SEQ ID NO: 302), G3744 (SEQ ID NO: 300), G3767 (SEQ ID NO: 298), G3768 (SEQ ID NO: 294), G3769 (SEQ ID NO: 296), G3765 (SEQ ID NO: 314), G3766 (SEQ ID NO: 304), G592 (SEQ ID NO: 306), G3086 (SEQ ID NO: 292), G3750 (SEQ ID NO: 326) and G3760 (SEQ ID NO: 324).

### DETAILED DESCRIPTION

The present invention relates to polynucleotides and polypeptides for modifying phenotypes of plants, particularly those associated with increased biomass, increased disease resistance, and/or abiotic stress tolerance. Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### DEFINITIONS

“Nucleic acid molecule” refers to an oligonucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA).

“Polynucleotide” is a nucleic acid molecule comprising a plurality of polymerized nucleotides, e.g., at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and that may be used to determine the limits of the genetically active unit (Rieger et al. (1976)). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “polypeptide” is an amino acid sequence comprising a plurality of consecutive polymerized amino acid residues e.g., at least about 15 consecutive polymerized amino acid residues. In many instances, a polypeptide comprises a polymerized amino acid residue sequence that is a transcription factor or a domain or portion or fragment thereof. Additionally, the polypeptide may comprise: (i) a localization domain; (ii) an activation domain; (iii) a repression domain; (iv) an oligomerization domain; (v) a DNA-binding domain; or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules which specifically bind to that portion or for the production of antibodies.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical, matching or corresponding nucleotides at positions shared by the polynucleotide sequences. A degree of identity of polypeptide sequences is a function of the number of identical amino acids at corresponding positions shared by the polypeptide sequences. A degree of homology or similarity of polypeptide sequences is a function of the number of amino acids at corresponding positions shared by the polypeptide sequences.

“Alignment” refers to a number of nucleotide bases or amino acid residue sequences aligned by lengthwise comparison so that components in common (i.e., nucleotide bases or amino acid residues at corresponding positions) may be visually and readily identified. The fraction or percentage of components in common is related to the homology or identity between the sequences. Alignments such as those of FIGS. 3A-3F may be used to identify conserved domains and relatedness within these domains. An alignment may suitably be determined by means of computer programs known in the art, such as MACVECTOR software (1999) (Accelrys, Inc., San Diego, Calif.).

A “conserved domain” or “conserved region” as used herein refers to a region in heterologous polynucleotide or polypeptide sequences where there is a relatively high degree of sequence identity between the distinct sequences. For example, an “AT-hook” domain”, such as is found in a polypeptide member of AT-hook transcription factor family, is an example of a conserved domain. An “AP2” domain”, such as is found in a polypeptide member of AP2 transcription factor family, is another example of a conserved domain. With respect to polynucleotides encoding presently disclosed transcription factors, a conserved domain is preferably at least nine base pairs (bp) in length. A conserved domain with respect to presently disclosed polypeptides refers to a domain within a transcription factor family that exhibits a higher degree of sequence homology, such as at least about 38% sequence identity including conservative substitutions, or at least about 55% sequence identity, or at least about 62% sequence identity, or at least about 65%, or at least about 70%, or at least about 75%, or at least about 78%, or at least about 80%, or at least about 82%, or at least about 85%, %, or at least about 90%, or at least about 95%, amino acid residue sequence identity, to a conserved domain of a polypeptide of the invention. Sequences that possess or encode for conserved domains that meet these criteria of percentage identity, and that have comparable biological activity to the present transcription factor sequences, thus being members of the G1073 clade of transcription factor polypeptides, are encompassed by the invention. A fragment or domain can be referred to as outside a conserved domain, outside a consensus sequence, or outside a consensus DNA-binding site that is known to exist or that exists for a particular transcription factor class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence (see, for example, Riechmann et al. (2000a, 2000b)). Thus, by using alignment methods well known in the art, the conserved domains of the plant transcription factors, for example, for the AT-hook proteins (Reeves and Beckerbauer (2001); and Reeves (2001)), may be determined.

The conserved domains for many of the transcription factor sequences of the invention are listed in Tables 8-17. Also, the polypeptides of Tables 8-17 have conserved domains specifically indicated by amino acid coordinate start and stop sites. A comparison of the regions of these polypeptides allows one of skill in the art (see, for example, Reeves and Nissen (1995)) to identify domains or conserved domains for any of the polypeptides listed or referred to in this disclosure.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985), Sambrook et al. (1989), and by Haymes et al. (1985), which references are incorporated herein by reference.

In general, stringency is determined by the temperature, ionic strength, and concentration of denaturing agents (e.g., formamide) used in a hybridization and washing procedure (for a more detailed description of establishing and determining stringency, see the section “Identifying Polynucleotides or Nucleic Acids by Hybridization”, below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known transcription factor sequences. Numerous variations are possible in the conditions and means by which nucleic acid hybridization can be performed to isolate transcription factor sequences having similarity to transcription factor sequences known in the art and are not limited to those explicitly disclosed herein. Such an approach may be used to isolate polynucleotide sequences having various degrees of similarity with disclosed transcription factor sequences, such as, for example, encoded transcription factors having 38% or greater identity with the conserved domain of disclosed transcription factors.

The terms “paralog” and “ortholog” are defined below in the section entitled “Orthologs and Paralogs”. In brief, orthologs and paralogs are evolutionarily related genes that have similar sequences and functions. Orthologs are structurally related genes in different species that are derived by a speciation event. Paralogs are structurally related genes within a single species that are derived by a duplication event.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) World Wide Web (www) website, “tigr.org” under the heading “Terms associated with TIGRFAMs”.

In general, the term “variant” refers to molecules with some differences, generated synthetically or naturally, in their base or amino acid sequences as compared to a reference (native) polynucleotide or polypeptide, respectively. These differences include substitutions, insertions, deletions or any desired combinations of such changes in a native polynucleotide of amino acid sequence.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations may result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a transcription factor nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the terms refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. Thus, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent transcription factor. Thus, it will be readily appreciated by those of skill in the art, that any of a variety of polynucleotide sequences is capable of encoding the transcription factors and transcription factor homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as a significant amount of the functional or biological activity of the transcription factor is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine. More rarely, a variant may have “non-conservative” changes, e.g., replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (see U.S. Pat. No. 5,840,544).

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, preferably at least about 30 nucleotides, more preferably at least about 50 nucleotides, of any of the sequences provided herein. Exemplary polynucleotide fragments are the first sixty consecutive nucleotides of the transcription factor polynucleotides listed in the Sequence Listing. Exemplary fragments also include fragments that comprise a region that encodes an conserved domain of a transcription factor. Exemplary fragments also include fragments that comprise a conserved domain of a transcription factor. Exemplary fragments include fragments that comprise an conserved domain of a transcription factor, for example, amino acid residues 33-77 of G682 (SEQ ID NO: 60).

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length.

The invention also encompasses production of DNA sequences that encode transcription factors and transcription factor derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding transcription factors or any fragment thereof.

“Derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae.

A “control plant” as used in the present invention refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence operably linked (i.e., under regulatory control of) to appropriate inducible or constitutive regulatory sequences that allow for the controlled expression of polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a transcription factor expression is altered, e.g., in that it has been knocked out, overexpressed, or ectopically expressed.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease, or an even greater difference, in an observed trait as compared with a control or wild-type plant. It is known that there can be a natural variation in the modified trait. Therefore, the trait modification observed entails a change of the normal distribution and magnitude of the trait in the plants as compared to control or wild-type plants.

When two or more plants have “similar morphologies”, “substantially similar morphologies”, “a morphology that is substantially similar”, or are “morphologically similar”, the plants have comparable forms or appearances, including analogous features such as overall dimensions, height, width, mass, root mass, shape, glossiness, color, stem diameter, leaf size, leaf dimension, leaf density, internode distance, branching, root branching, number and form of inflorescences, and other macroscopic characteristics, and the individual plants are not readily distinguishable based on morphological characteristics alone.

“Modulates” refers to a change in activity (biological, chemical, or immunological) or lifespan resulting from specific binding between a molecule and either a nucleic acid molecule or a protein.

The term “transcript profile” refers to the expression levels of a set of genes in a cell in a particular state, particularly by comparison with the expression levels of that same set of genes in a cell of the same type in a reference state. For example, the transcript profile of a particular transcription factor in a suspension cell is the expression levels of a set of genes in a cell knocking out or overexpressing that transcription factor compared with the expression levels of that same set of genes in a suspension cell that has normal levels of that transcription factor. The transcript profile can be presented as a list of those genes whose expression level is significantly different between the two treatments, and the difference ratios. Differences and similarities between expression levels may also be evaluated and calculated using statistical and clustering methods.

With regard to transcription factor gene knockouts as used herein, the term “knockout” refers to a plant or plant cell having a disruption in at least one transcription factor gene in the plant or cell, where the disruption results in a reduced expression or activity of the transcription factor encoded by that gene compared to a control cell. The knockout can be the result of, for example, genomic disruptions, including transposons, tilling, and homologous recombination, antisense constructs, sense constructs, RNA silencing constructs, or RNA interference. A T-DNA insertion within a transcription factor gene is an example of a genotypic alteration that may abolish expression of that transcription factor gene.

“Ectopic expression or altered expression” in reference to a polynucleotide indicates that the pattern of expression in, e.g., a transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more transcription factors are under the control of a strong promoter (e.g., the cauliflower mosaic virus 35S transcription initiation region). Overexpression may also under the control of an inducible or tissue specific promoter. Thus, overexpression may occur throughout a plant, in specific tissues of the plant, or in the presence or absence of particular environmental signals, depending on the promoter used.

Overexpression may take place in plant cells normally lacking expression of polypeptides functionally equivalent or identical to the present transcription factors. Overexpression may also occur in plant cells where endogenous expression of the present transcription factors or functionally equivalent molecules normally occurs, but such normal expression is at a lower level. Overexpression thus results in a greater than normal production, or “overproduction” of the transcription factor in the plant, cell or tissue.

The term “transcription regulating region” refers to a DNA regulatory sequence that regulates expression of one or more genes in a plant when a transcription factor having one or more specific binding domains binds to the DNA regulatory sequence. Transcription factors of the present invention possess an conserved domain. The transcription factors of the invention also comprise an amino acid subsequence that forms a transcription activation domain that regulates expression of one or more abiotic stress tolerance genes in a plant when the transcription factor binds to the regulating region.

### DESCRIPTION OF THE SPECIFIC EMBODIMENTS

### Transcription Factors Modify Expression of Endogenous Genes

A transcription factor may include, but is not limited to, any polypeptide that can activate or repress transcription of a single gene or a number of genes. As one of ordinary skill in the art recognizes, transcription factors can be identified by the presence of a region or domain of structural similarity or identity to a specific consensus sequence or the presence of a specific consensus DNA-binding site or DNA-binding site motif (see, for example, Riechmann et al. (2000a)). The plant transcription factors of the present invention belong to the AT-hook transcription factor family (Reeves and Beckerbauer (2001); and Reeves (2001)).

Generally, the transcription factors encoded by the present sequences are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with improved traits related to osmotic stresses. The sequences of the invention may also be used to transform a plant and introduce desirable traits not found in the wild-type cultivar or strain. Plants may then be selected for those that produce the most desirable degree of over- or under-expression of target genes of interest and coincident trait improvement.

The sequences of the present invention may be from any species, particularly plant species, in a naturally occurring form or from any source whether natural, synthetic, semi-synthetic or recombinant. The sequences of the invention may also include fragments of the present amino acid sequences. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

In addition to methods for modifying a plant phenotype by employing one or more polynucleotides and polypeptides of the invention described herein, the polynucleotides and polypeptides of the invention have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, e.g., mutation reactions, PCR reactions, or the like; as substrates for cloning e.g., including digestion or ligation reactions; and for identifying exogenous or endogenous modulators of the transcription factors. The polynucleotide can be, e.g., genomic DNA or RNA, a transcript (such as an mRNA), a cDNA, a PCR product, a cloned DNA, a synthetic DNA or RNA, or the like. The polynucleotide can comprise a sequence in either sense or antisense orientations.

Expression of genes that encode transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) and Peng et al. (1999). In addition, many others have demonstrated that an Arabidopsis transcription factor expressed in an exogenous plant species elicits the same or very similar phenotypic response. See, for example, Fu et al. (2001); Nandi et al. (2000); Coupland (1995); and Weigel and Nilsson (1995)).

In another example, Mandel et al. (1992), and Suzuki et al. (2001), teach that a transcription factor expressed in another plant species elicits the same or very similar phenotypic response of the endogenous sequence, as often predicted in earlier studies of Arabidopsis transcription factors in Arabidopsis (see Mandel et al. (1992); Suzuki et al. (2001)). Other examples include Müller et al. (2001); Kim et al. (2001); Kyozuka and Shimamoto (2002); Boss and Thomas (2002); He et al. (2000); and Robson et al. (2001).

In yet another example, Gilmour et al. (1998) teach an Arabidopsis AP2 transcription factor, CBF1, which, when overexpressed in transgenic plants, increases plant freezing tolerance. Jaglo et al. (2001) further identified sequences in Brassica napus which encode CBF-like genes and that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues, PKK/RPAGRxKFxETRIP and DSAWR, which bracket the AP2/EREBP DNA binding domains of the proteins and distinguish them from other members of the AP2/EREBP protein family. (Jaglo et al. (2001))

Transcription factors mediate cellular responses and control traits through altered expression of genes containing cis-acting nucleotide sequences that are targets of the introduced transcription factor. It is well appreciated in the art that the effect of a transcription factor on cellular responses or a cellular trait is determined by the particular genes whose expression is either directly or indirectly (e.g., by a cascade of transcription factor binding events and transcriptional changes) altered by transcription factor binding. In a global analysis of transcription comparing a standard condition with one in which a transcription factor is overexpressed, the resulting transcript profile associated with transcription factor overexpression is related to the trait or cellular process controlled by that transcription factor. For example, the PAP2 gene and other genes in the MYB family have been shown to control anthocyanin biosynthesis through regulation of the expression of genes known to be involved in the anthocyanin biosynthetic pathway (Bruce et al. (2000); and Borevitz et al. (2000)). Further, global transcript profiles have been used successfully as diagnostic tools for specific cellular states (e.g., cancerous vs. non-cancerous; Bhattachaijee et al. (2001); and Xu et al. (2001)). Consequently, it is evident to one skilled in the art that similarity of transcript profile upon overexpression of different transcription factors would indicate similarity of transcription factor function.

Polypeptides and Polyucleotides of the Invention

The present invention provides, among other things, transcription factors (TFs), and transcription factor homolog polypeptides, and isolated or recombinant polynucleotides encoding the polypeptides, or novel sequence variant polypeptides or polynucleotides encoding novel variants of transcription factors derived from the specific sequence provided in the Sequence Listing. Also provided are methods for modifying a plant's biomass by modifying the size or number of leaves or seed of a plant by controlling a number of cellular processes, and for increasing a plant's resistance or tolerance to disease or abiotic stresses, respectively. These methods are based on the ability to alter the expression of critical regulatory molecules that may be conserved between diverse plant species. Related conserved regulatory molecules may be originally discovered in a model system such as Arabidopsis and homologous, functional molecules then discovered in other plant species. The latter may then be used to confer increased biomass, disease resistance or abiotic stress tolerance in diverse plant species.

Exemplary polynucleotides encoding the polypeptides of the invention were identified in the Arabidopsis thaliana GenBank database using publicly available sequence analysis programs and parameters. Sequences initially identified were then further characterized to identify sequences comprising specified sequence strings corresponding to sequence motifs present in families of known transcription factors. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known transcription factors. Polynucleotide sequences meeting such criteria were confirmed as transcription factors.

Additional polynucleotides of the invention were identified by screening Arabidopsis thaliana and/or other plant cDNA libraries with probes corresponding to known transcription factors under low stringency hybridization conditions. Additional sequences, including full length coding sequences, were subsequently recovered by the rapid amplification of cDNA ends (RACE) procedure using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

Many of the sequences in the Sequence Listing, derived from diverse plant species, have been ectopically expressed in overexpressor plants. The changes in the characteristic(s) or trait(s) of the plants were then observed and found to confer increased disease resistance, increase biomass and/or increased abiotic stress tolerance. Therefore, the polynucleotides and polypeptides can be used to improve desirable characteristics of plants.

The polynucleotides of the invention were also ectopically expressed in overexpressor plant cells and the changes in the expression levels of a number of genes, polynucleotides, and/or proteins of the plant cells observed. Therefore, the polynucleotides and polypeptides can be used to change expression levels of a genes, polynucleotides, and/or proteins of plants or plant cells.

### DESCRIPTION OF THE SPECIFIC EMBODIMENTS

The data presented herein represent the results obtained in experiments with transcription factor polynucleotides and polypeptides that may be expressed in plants for the purpose of reducing yield losses that arise from biotic and abiotic stress.

### Background Information for the G482 Clade, Including G481 and Related Sequences

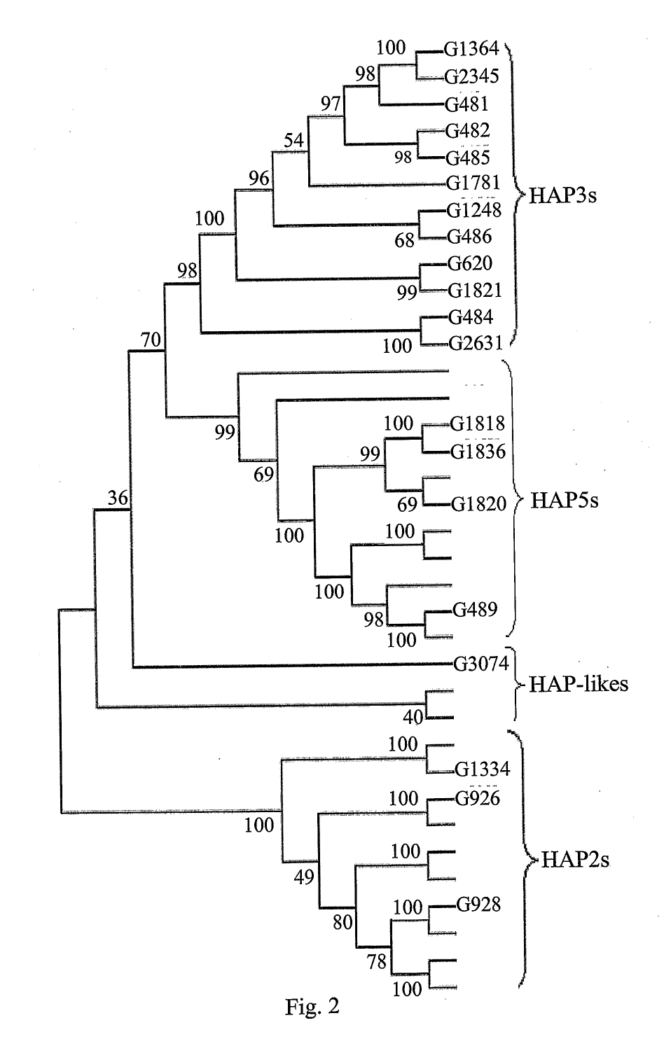

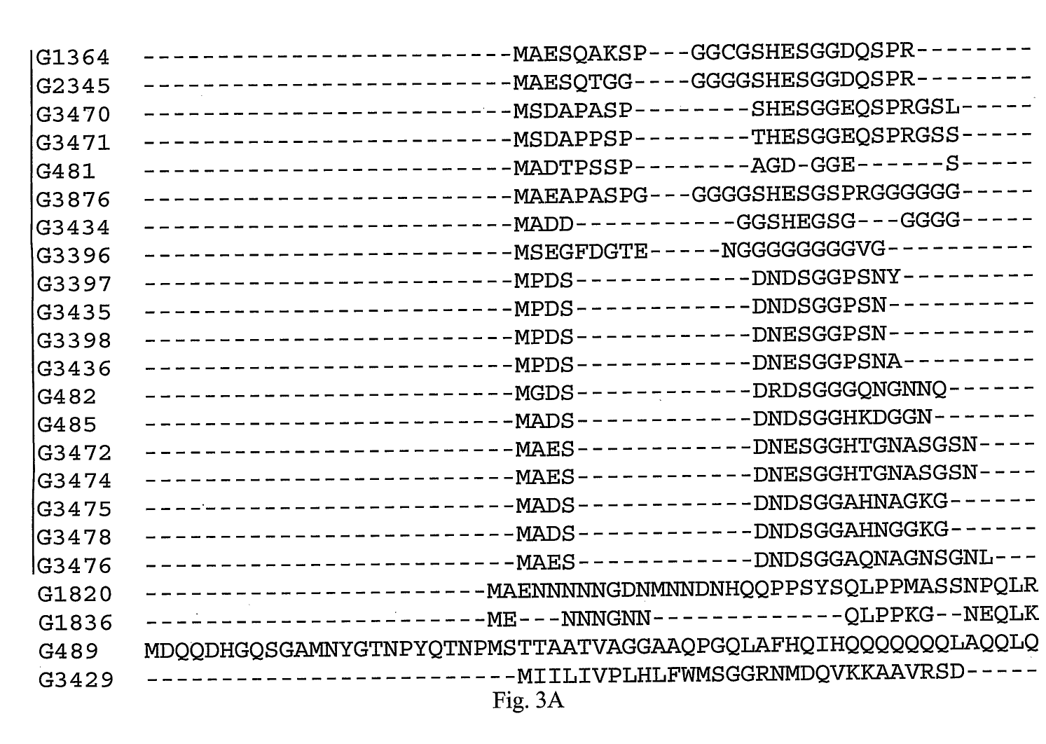

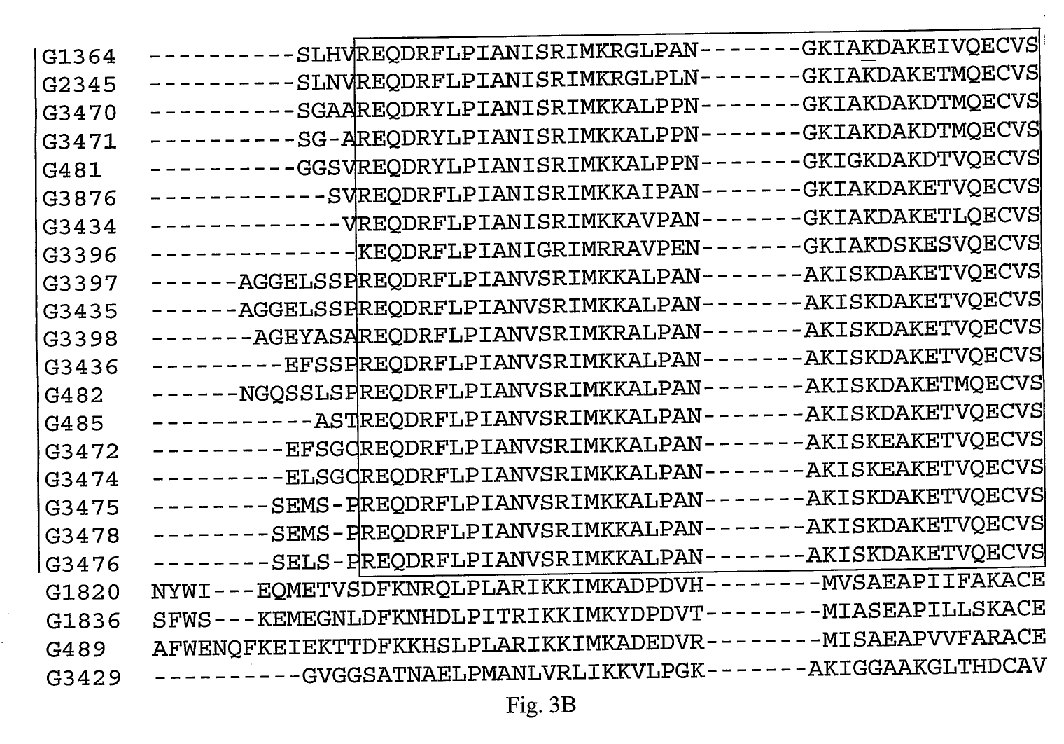

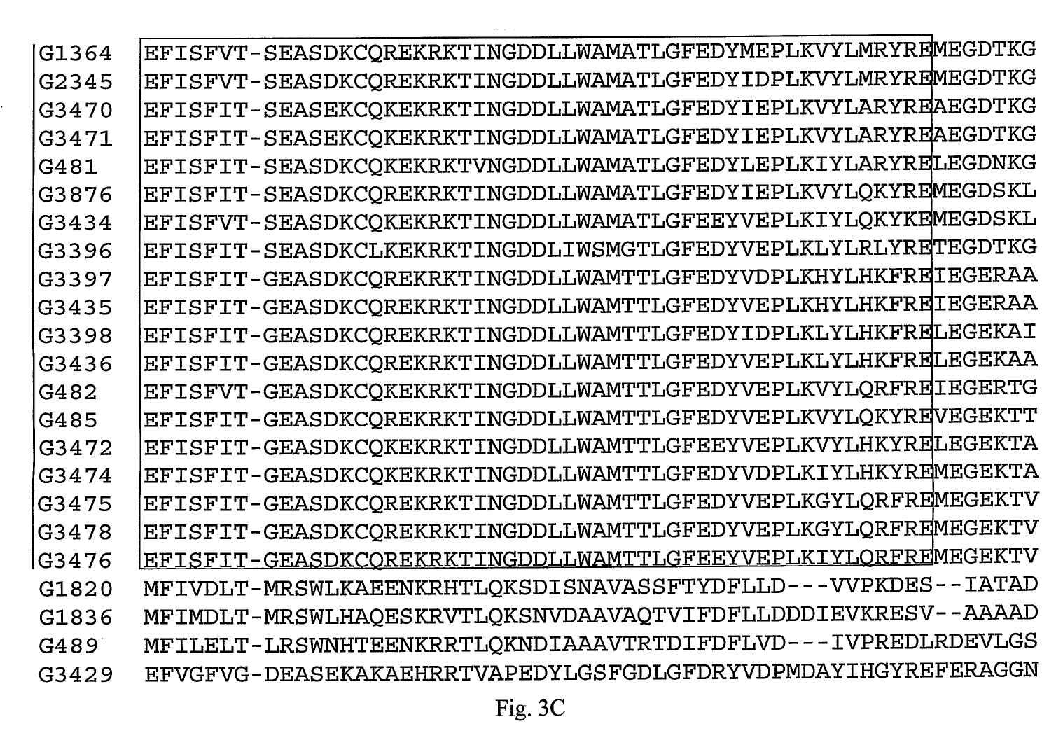

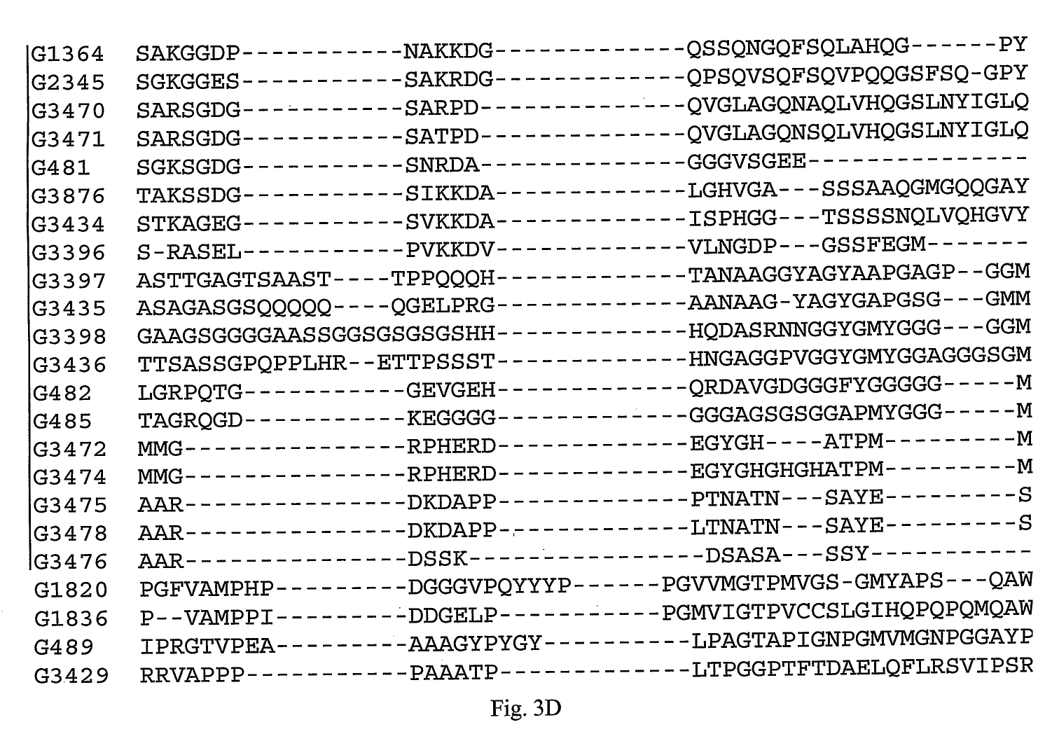

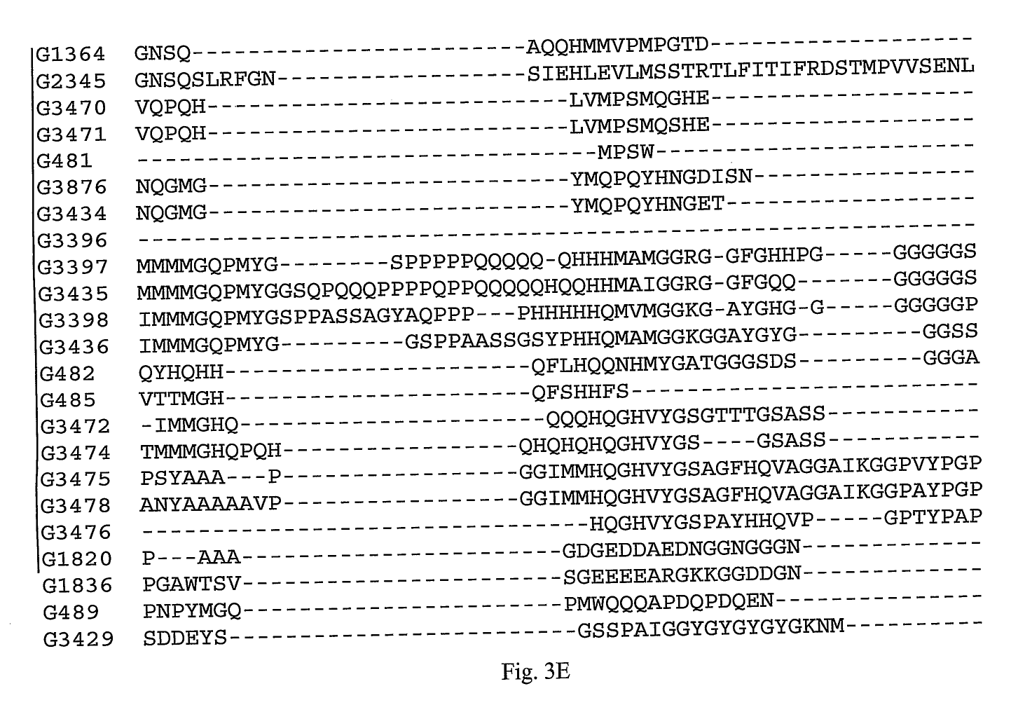

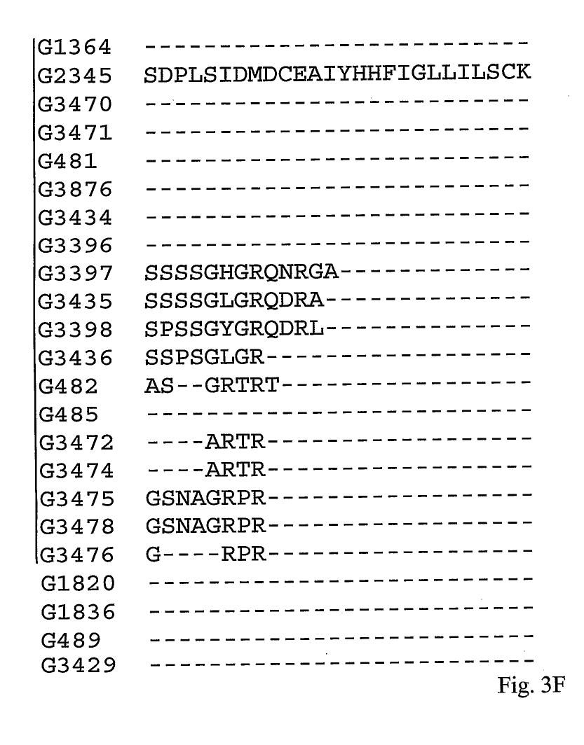

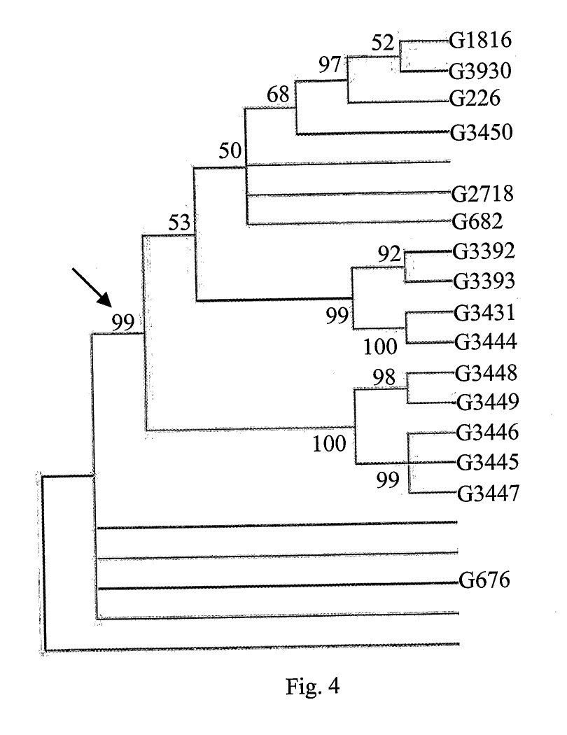

G481 (SEQ ID NOs: 1 and 2; AT2G38880; also known as HAP3A and AT-YB1) from Arabidopsis is a member of the HAP3/NF-YB sub-group of the CCAAT binding factor family (CCAAT) of transcription factors (FIG. 2). This gene was included based on the resistance to drought-related abiotic stress exhibited by 35S::G481 lines. The major goal of the current program is to define the mechanisms by which G481 confers drought tolerance, and to determine the extent to which other proteins from the CCAAT family, both in Arabidopsis and other plant species, have similar functions.

Structural features and assembly of the NF-Y subunits. NF-Y is one of the most heavily studied transcription factor complexes and an extensive literature has accumulated regarding its structure, regulation, and putative roles in various different organisms. Each of the three subunits comprises a region which has been evolutionarily conserved (Li et al. (1992); Mantovani (1999)). In the NF-YA subunits, this conserved region is at the C-terminus, in the NF-YB proteins it is centrally located, and in the NF-YC subunits it is at the N-terminus. The NF-YA and NF-YC subunits also have regions which are rich in glutamine (Q) residues that also show some degree of conservation; these Q-rich regions have an activation domain function. In fact it has been shown that NF-Y contains two transcription activation domains: a glutamine-rich, serine-threonine-rich domain present in the CBF-B (HAP2, NF-YA) subunit and a glutamine-rich domain in the CBF-C (HAP5, CBF-C) subunit (Coustry et al. (1995); Coustry et al. (1996); Coustry et al. (1998); Coustry et al. (2001)). In yeast, Q-regions are not present in the NF-Y subunits and the activation function is thought to be provided by an acidic region in HAP4 (Forsburg and Guarente (1989); Olesen and Guarente (1990); McNabb et al. (1997)), the subunit that is absent from mammals.

The NF-YB and NF-YC subunits bear some similarity to histones; the conserved regions of both these subunits contain a histone fold motif (HFM), which is an ancient domain of ˜65 amino acids. The HFM has a high degree of structural conservation across all histones and comprises three or four α-helices (four in the case of the NF-Y subunits) which are separated by short loops (L)/strand regions (Arents and Moudrianakis (1995)). In the histones, this HFM domain mediates dimerization and formation of non sequence-specific interactions with DNA (Arents and Moudrianakis (1995)).

Considerable knowledge has now accumulated regarding the biochemistry of NF-Y subunit association and DNA binding. The NF-YB-NF-YC subunits first form a tight dimer, which offers a complex surface for NF-YA association. The resulting trimer can then bind to DNA with high specificity and affinity (Kim and Sheffrey (1990); Bi et al. (1997); Mantovani (1999)). In addition to the NF-Y subunits themselves, a number of other proteins have been implicated in formation of the complex (Mantovani (1999)).

Using approaches such as directed mutagenesis, specific regions of the NF-Y proteins have been altered and inferences made about their specific role. In particular, it is has been found that the HFMs of NF-YB and NF-YC are critical for dimer formation, NF-YA association and CCAAT-binding (Sinha et al. (1996); Kim et al. (1996); Xing et al. (1993); Maity and de Crombrugghe (1998)). Specific amino acids in α2, L2 and α3 are required for dimerization between the NF-YB and NF-YC subunits. For NF-YA association, two conserved amino acids in α2 from NF-YB and several residues in NF-YC, within α1, α2 and at the C-terminus of α3 are required. For DNA binding, which is the most difficult feature to address since the two other functions need to be intact, the α1 and α2 of NF-YB and the α1 of NF-YC are necessary. These latter results do not rule out that other parts of the HFMs are necessary to make the trimer bind to DNA; most notably the positively charged residues in L2 may have such a role, as in histones (Luger et al. (1997); Mantovani (1999)).

Most of the sequence specific interactions within the NF-Y trimer appear to be conferred by NF-YA. In contrast to the B and C subunits, the conserved domain in the A subunit does not bear any resemblance to histones or any other well-characterized DNA-binding motif. However, like the B and C subunits, the A subunit has also been subject to saturation mutagenesis. The NF-YA conserved domain appears to comprise two distinct halves, each of ˜20 amino acids; the N-terminal part of the conserved domain is required for association with the BC dimer, whereas the C-terminal portion of the NF-YA conserved domain is needed for DNA binding (Mantovani (1999)), Further structural insights into the function of NF-Y have now been obtained following solution of the crystal structure of the BC dimer (Romier et al. (2003)). This confirmed the role of the HFM motifs and the role of the conserved regions of NF-YA; a model for DNA interactions suggests that the NF-YA subunit binds the CCAAT box while the B and C subunits bend the DNA (Romier et al. (2003)).

There is very little sequence similarity between HAP3 proteins in the A and C domains; it is therefore reasonable to assume that the A and C domains could provide a degree of functional specificity to each member of the HAP3 subfamily. The B domain is the conserved region that specifies DNA binding and subunit association.

In FIGS. 3A-3F, HAP3 proteins from Arabidopsis, soybean, rice and corn are aligned with G481. The B domain of the non-LEC1-like clade (identified in the box spanning FIGS. 3B-3C) may be distinguished by the comprised amino acid residues:

Asn-(Xaa)₄₋₁₁-Lys-(Xaa)₃₃₋₃₄-Asn-Gly-(Xaa)₂-Leu;

where Xaa can be any amino acid. These residues in their present positions are uniquely found in the non-LEC1-like clade, and may be used to identify members of this clade.

The G482 subclade is distinguished by a B-domain comprising:

Ser/Glu-(Xaa)₉-Asn-(Xaa)₄₋₁₁-Lys-(Xaa)₃₃₋₃₄-Asn-
Gly-(Xaa)₂-Leu.

Plant CCAAT binding factors are regulated at the level of transcription. In contrast to the NF-Y genes from mammals, members of the CCAAT family from Arabidopsis appear to be heavily regulated at the level of RNA abundance. Surveys of expression patterns of Arabidopsis CCAAT family members from a number of different studies have revealed complex patterns of expression, with some family members being specific to particular tissue types or conditions (Edwards et al. (1998); Gusmaroli et al. (2001); Gusmaroli et al. (2002)). During previous genomics studies, we also found that the expression patterns of many of the HAP-like genes in Arabidopsis were suggestive of developmental and/or conditional regulation. In particular LEC1 (G6201, SEQ ID NO: 357) and L1L (G1821, SEQ ID NO: 358) were very strongly expressed in siliques and embryos relative to other tissues. We used RT-PCR to analyze the endogenous expression of 31 of the 36 CCAAT-box genes. Our findings suggested that while many of the CCAAT-box gene transcripts are found ubiquitously throughout the plant, in more than half of the cases, the genes are predominantly expressed in flower, embryo and/or silique tissues.

Roles of CCAAT binding factors in plants. The specific roles of CCAAT-box elements and their binding factors in plants are still poorly understood. CCAAT-box elements have been shown to function in the regulation of gene expression (Rieping and Schoffl (1992); Kehoe et al. (1994); Ito et al. (1995)). Several reports have described the importance of the CCAAT-binding element for regulated gene expression; including the modulation of genes that are responsive to light (Kusnetsov et al. (1999); Carre and Kay (1995); Bezhani et al. (2001)) as well as stress (Rieping and Schoffl (1992)). Specifically, a CCAAT-box motif was shown to be important for the light regulated expression of the CAB2 promoter in Arabidopsis. However, the proteins that bind to the site were not identified (Carre and Kay (1995)).

Role of LEC1-like proteins. The functions of only two of the Arabidopsis CCAAT-box genes have been genetically determined in the public domain. These genes, LEAFY COTYLEDON 1 (LEC1, G620, SEQ ID NO: 357) and LEAFY COTYLEDON 1-LIKE (L1L, G1821, SEQ ID NO: 358) have critical roles in embryo development and seed maturation (Lotan et al. (1998); Kwong et al. (2003)) and encode proteins of the HAP3 (NF-YB) class. LEC1 has multiple roles in and is critical for normal development during both the early and late phases of embryogenesis (Meinke (1992); Meinke et al. (1994); West et al. (1994); Parcy et al. (1997); Vicient et al. (2000)), Mutant lec1 embryos have cotyledons that exhibit leaf-like characteristics such as trichomes. The gene is required to maintain suspensor cell fate and to specify cotyledon identity in the early morphogenesis phase. Through overexpression studies, LEC1 activity has been shown sufficient to initiate embryo development in vegetative cells (Lotan et al. (1998)). Additionally, lec1 mutant embryos are desiccation intolerant and cannot survive seed dry-down (but can be artificially rescued in the laboratory). This phenotype reflects a role for LEC1 at later stages of seed maturation; the gene initiates and/or maintains the maturation phase, prevents precocious germination, and is required for acquisition of desiccation tolerance during seed maturation. L1L appears to be a paralog of, and partially redundant with LEC1. Like LEC1, L1L is expressed during embryogenesis, and genetic studies have demonstrated that L1L can complement lec1 mutants (Kwong et al. (2003)).

Putative LEC1 orthologs exist in a wide range of species and based on expression patterns, likely have a comparable function to the Arabidopsis gene. For example, the ortholog of LEC1 has been identified recently in maize. The expression pattern of ZmLEC1 in maize during somatic embryo development is similar to that of LEC1 in Arabidopsis during zygotic embryo development (Zhang et al. (2002)). A comparison of LEC1-like proteins with other proteins of the HAP3 sub-group indicates that the LEC1-like proteins form a distinct phylogenetic clade, and have a number of distinguishing residues, which set them apart from the non-LEC1-like HAP3 proteins (Kwong et al. (2003)). Thus it is likely that the LEC1 like proteins have very distinct functions compared to proteins of the non-LEC1-like HAP3 group.

HAP3 (NF-YB) proteins have a modular structure and are comprised of three distinct domains: an amino-terminal A domain, a central B domain and a carboxy-terminal C domain. There is very little sequence similarity between HAP3 proteins within the A and C domains suggesting that those regions could provide a degree of functional specificity to each member of the HAP3 subfamily. The B domain is a highly conserved region that specifies DNA binding and subunit association. Lee et al. (2003) performed an elegant series of domain swap experiments between the LEC1 and a non-LEC1 like HAP3 protein (At4 g14540, G485) to demonstrate that the B domain of LEC1 is necessary and sufficient, within the context of the rest of the protein, to confer its activity in embryogenesis. Furthermore, these authors identified a specific defining residue within the B domain (Asp-55) that is required for LEC1 activity and which is sufficient to confer LEC1 function to a non-LEC1 like B domain.

Discoveries made in earlier genomics programs. G481 is a member of the HAP3 (NF-YB) group of CCAAT-box binding proteins, and falls within the non-LEC-like clade of proteins. G481 is equivalent to AtHAP3a, which was identified by Edwards et al. (1998), as an EST with extensive sequence homology to the yeast HAP3. Northern blot data from five different tissue samples indicated that G481 is primarily expressed in flower and/or silique, and root tissue. RT-PCR studies partially confirmed the published expression data; we detected relatively low levels of G481 expression in all of the tissues tested, with somewhat higher levels of expression being detected in flowers, siliques, and embryos. However, the differential expression of G481 in these relative to other tissues was much less dramatic than that which was seen for G620 (LEC1, 1, SEQ ID NO: 357) and G1821 (L1L, 1, SEQ ID NO: 358), which function specifically in embryo development.

It was initially discovered that 35S::G481 lines display a hyperosmotic stress tolerance and/or sugar sensing phenotype on media containing high levels of sucrose, after which drought tolerance in a soil-based assay was demonstrated. In addition to G481, there are a further seven other non-LEC1-like proteins which lie on the same branch of the phylogenetic tree (FIG. 2), and represent the phylogenetically related sequences G1364, G2345, G482, G485, G1781, G1248 and G486 (polypeptide SEQ ID NOs: 14, 22, 28, 18, 56, 360, and 356, respectively). Two other HAP3 proteins, G484 (polypeptide SEQ ID NO: 354) and G2631 (polypeptide SEQ ID NO: 362) appear to be rather more distantly related. G1364 and G2345 are the Arabidopsis proteins most closely related to G481; however, neither of these genes has been found to confer hyperosmotic stress tolerance.

G482 (polypeptide SEQ ID NO: 28) is slightly further diverged from G481 than G2345 and G1364 (FIG. 2), but has an apparently similar function given that 35S::G482 lines analyzed during our initial genomics screens displayed an hyperosmotic stress response phenotype similar to 35S::G481. Another HAP3 gene, G485 (SEQ ID NO: 17 and 18), is most closely related to G482. G485 was not implicated in regulation of stress responses in our initial screens, but KO.G485 and 35S::G485 lines exhibited opposite flowering time phenotypes, with the mutant flowering late, and the overexpression lines flowering early. Thus, G485 functions as an activator of the floral transition. Interestingly, two of the other non-LEC1-like genes, G1781 (SEQ ID NO: 55) and G1248 (SEQ ID NO: 359), were also found to accelerate flowering when overexpressed, during our genomics program. However, overexpression lines for neither of those genes were found to show alterations in stress tolerance. G486 was also noted to produce effects on flowering time, but these were inconclusive and rather variable between different lines.

In addition to HAP3 (NF-YB) genes, a number of HAP5 (NF-YC) genes were found to influence abiotic stress responses during our initial genomics program. G489 (SEQ ID NO: 45), G1836 (SEQ ID NO: 47), and G1820 (SEQ ID NO: 43) are all HAP5-like proteins that generated hyperosmotic stress tolerance phenotypes when overexpressed. Thus, we surmised that these proteins might potentially be members of the same heteromeric complex as G481 or one or more of the other HAP3 proteins.

Potential mode of action of G481. The enhanced tolerance of 35S::G481 lines to sucrose seen in our genomics screens suggests that G481 could influence sugar sensing and hormone signaling. Several sugar sensing mutants have turned out to be allelic to ABA and ethylene mutants. On the other hand, the sucrose treatment (9.5% w/v) could have represented an hyperosmotic stress; thus, one might also interpret the results as indicating that G481 confers tolerance to hyperosmotic stress. LEC1 (G620, polypeptide SEQ ID NO: 358), which is required for desiccation tolerance during seed maturation, is also ABA and drought inducible. This information, combined with the fact that CCAAT genes are disproportionately responsive to hyperosmotic stress suggests that the family could control pathways involved in both ABA response and desiccation tolerance. In particular, given their phylogenetic divergence, it is possible that LEC1-like proteins have evolved to confer desiccation tolerance specifically within the embryo, whereas other non-LEC1-like HAP3 proteins confer tolerance in non-embryonic tissues.

A role in sugar sensing also supports the possibility that, as in yeast, CCAAT-box factors from plants play a general role in the regulation of energy metabolism. Indeed, the fact that plants exhibit two modes of energy metabolism (in the form of photosynthesis and respiration) could account for the expansion of the family in the plant kingdom. Specifically, a mechanism that is currently being evaluated is that G481-related proteins regulate starch/sugar metabolism, and as such, influence both the osmotic balance of cells as well as the supply of photosynthate to sink areas. Such hypotheses can account for a number of the off-types, such as reduced yield (under well-watered conditions) and delayed senescence, seen in corn and soy field tests of G481 (and related genes) overexpression lines. The prospective involvement of CCAAT box factors in chloroplast development and retrograde signaling also suggests a further means by which G481-related genes could confer stress tolerance. The genes might act to maintain chloroplast function under unfavorable conditions. In fact, any effects on expression of chloroplast components could well be indirectly related to the putative effects on carbohydrate metabolism.

### Background Information for G634, the G634 Clade, and Related Sequences

G634 (SEQ ID NO: 49) encodes a TH family protein (SEQ ID NO: 50). This gene was initially identified from public partial cDNAs sequences for GTL1 and GTL2 which are splice variants of the same gene (Smalle et al (1998)). The published expression pattern of GTL1 shows that G634 is highly expressed in siliques and not expressed in leaves, stems, flowers or roots.

### Background Information for G1073, the G1073 Clade, and Related Sequences

G1073 (SEQ ID NO: 114) is a member of the At-hook family of transcription factors. We have now designated this locus as HERCULES 1 (HRC1), in recognition of the increased organ size seen in 35S::G1073 lines. A major goal of the current program is to define the mechanisms by which G1073 regulates organ growth and to understand how these are related to the ability of this factor to regulate stress tolerance responses. This will allow us to optimize the gene for use in particular target species where increased stress tolerance is desired without any associated effects on growth and development.

Structural features of the G1073 protein. G1073 is a 299 residue protein that contains a single typical AT-hook DNA-binding motif (RRPRGRPAG) at amino acids 63 to 71. A highly conserved 129 AA domain, with unknown function, can be identified in the single AT-hook domain subgroup. Following this region, a potential acidic domain spans from position 200 to 219. Additionally, analysis of the protein using PROSITE reveals three potential protein kinase C phosphorylation sites at Ser61, Thr112 and Thr131, and three potential casein kinase II phosphorylation sites at Ser35, Ser99 and Ser276. Additional structural features of G1073 include 1) a short glutamine-rich stretch in the C-terminal region distal to the conserved acidic domain, and 2) possible PEST sequences in the same C-terminal region.

The G1073 protein is apparently shorter at the N-terminus compared to many of the related At-hook proteins that we had identified. The product of the full-length cDNA for G1073 (SEQ ID NO: 113, polypeptide product SEQ ID NO: 114 and shown in FIGS. 10A-10H) has an additional 29 amino acids at the N-terminus relative to our original clone P448, SEQ ID NO: 609, was the original G1073 clone that was overexpressed during earlier genomics screens). We have now built a new phylogenetic tree for G1073 versus the related proteins, but the relationships on this new tree are not substantially changed relative to phylogeny presented in our previous studies.

With regard to G1073 and related sequences, within the G1073 clade of transcription factor polypeptides the AT-hook domain generally comprises the consensus sequence:

RPRGRPXG,
or
Arg-Pro-Arg-Gly-Arg-Pro-Xaa-Gly

where X or Xaa can be any of a number of amino acid residues; in the examples that have thus far been shown to confer abiotic stress tolerance, Xaa has been shown to represent an alanine, leucine, proline, or serine residue.

Also within the G1073 clade, a second conserved domain exists that generally comprises the consensus sequence:

Gly-Xaa-Phe-Xaa-Ile-Leu-Ser-(Xaa)₂-Gly-(Xaa)₂-Leu-
Pro-(Xaa)₃₋₄-Pro-(Xaa)₅-Leu-(Xaa)₂-Tyr/Phe-(Xaa)₂-
Gly-(Xaa)₂-Gly-Gln.

A smaller subsequence of interest in the G1073 clade sequences comprises:

Pro-(Xaa)₅-Leu-(Xaa)₂-Tyr;
or
Pro-(Xaa)₅-Leu-(Xaa)₂-Phe;

The tenth position of these latter two sequences is an aromatic residue, specifically tyrosine or phenylalanine, in the G1073 clade sequences that have thus far been examined.

Thus, the transcription factors of the invention each possess an AT-hook domain and a second conserved domain, and include paralogs and orthologs of G1073 found by BLAST analysis, as described below. The AT-hook domains of G1073 and related sequences examined thus far are at least 56% identical to the At-Hook domains of G1073, and the second conserved domains of these related sequences are at least 44% identical to the second conserved domain found in G1073. These transcription factors rely on the binding specificity of their AT-hook domains; many have been shown to have similar or identical functions in plants by increasing the size and biomass of a plant.

Role of At-hook proteins. The At-hook is a short, highly-conserved, DNA binding protein motif that comprises a conserved nine amino acid peptide (KRPRGRPKK) and is capable of binding to the minor groove of DNA (Reeves and Nissen (1990)). At the center of this AT-hook motif is a short, strongly conserved tripeptide (GRP) comprised of glycine-arginine-proline (Aravind and Landsman (1998)). At-hook motifs were first recognized in the non-histone chromosomal protein HMG-I(Y) but have since been found in other DNA binding proteins from a wide range of organisms. In general, it appears that the AT-hook motif is an auxiliary protein motif cooperating with other DNA-binding activities and facilitating changes in the structure of the chromatin (Aravind and Landsman (1998)). The AT-hook motif can be present in a variable number of copies (1-15) in a given AT-hook protein. For example, the mammalian HMG-I(Y) proteins have three copies of this motif.

In higher organisms, genomic DNA is assembled into multilevel complexes by a range of DNA-binding proteins, including the well-known histones and non-histone proteins such as the high mobility group (HMG) proteins (Bianchi and Beltrame (2000)). HMG proteins are classified into different groups based on their DNA-binding motifs, and it is the proteins from one such group, the HMG-I(Y) subgroup, which are all characterized by the presence of copies of the At-hook. (Note that the HMG-I(Y) subgroup was recently renamed as HMGA; see Table 1 of report in Bianchi and Beltrame (2000), for information on nomenclature).

HMGA class proteins containing AT-hook domains have also been identified in a variety of plant species, including rice, pea and Arabidopsis (Meijer et al. (1996); and Gupta et al (1997a)). Depending on the species, plant genomes contain either one or two genes that encode HMGA proteins. In contrast to the mammalian HMGA proteins, though, the plant HMGA proteins usually possess four, rather than three repeats of the At-hook (see reviews by Grasser (1995); Grasser (2003)). Typically, plant HMGA genes are expressed ubiquitously, but the level of expression appears to be correlated with the proliferative state of the cells. For example, the rice HMGA genes are predominantly expressed in young and meristematic tissues and may affect the expression of genes that determine the differentiation status of cells. The pea HMGA gene is expressed in all organs including roots, stems, leaves, flowers, tendrils and developing seeds (Gupta et al (1997a)). Northern blot analysis revealed that an Arabidopsis HMGA gene was expressed in all organs with the highest expression in flowers and developing siliques (Gupta et al. (1997b)).

In plants, however, very little is known about the specific roles of HMGA class proteins. Nonetheless, there is some evidence that they might have functions in regulation of light responses. For example, PF1, a protein with AT-hook DNA-binding motifs from oat and was shown to binds to the PE1 region in the oat phytochrome A3 gene promoter. This factor and may be involved in positive regulation of PHYA3 gene expression (Nieto-Sotelo and Quail (1994)). The same group later demonstrated that PF1 from pea interacts with the PHYA gene promoter and stimulates binding of the transcriptional activator GT-2 (Martinez-Garcia and Quail (1999)). Another example concerns expression of a maize AT-hook protein in yeast cells, which produced better growth on a medium containing high nickel concentrations. Such an effect suggests that the protein might influence chromatin structure, and thereby restrict nickel ion accessibility to DNA (Forzani et al. (2001)).

During our genomics program we identified 34 Arabidopsis genes that code for proteins with AT-hook DNA-binding motifs. Of these proteins, 22 have a single AT-hook DNA-binding motif; 8 have two AT-hook DNA-binding motifs; three (G280, G1367 and G2787, SEQ ID NOs: 364, 366 and 370, respectively) have four AT-hook DNA-binding motifs. The public data regarding the function of these factors are sparse. This is particularly true of those proteins containing single AT-hook motifs such as G1073. It is worth noting that these single At-hook factors may function differently to those with multiple AT-hook motifs, such as HMGA proteins. However, an activation-tagged mutant for an Arabidopsis AT-hook gene named ESCAROLA (corresponding to G1067) has been identified by Weigel et al. (Weigel et al. (2000)). In this G1067 activation line, delayed flowering was observed, and leaves were wavy, dark green, larger, and rounder than in wild type. Moreover, both leaf petioles and stem internodes were shorter in this line than wild type. Such complex phenotypes suggest that the gene influences a wide range of developmental processes.

Recently, one of the single At-hook class proteins has been shown to have a structural role in the nucleus. At-hook motif nuclear localized protein (AHL1), corresponding to G1944, SEQ ID NO: 3687, was found in the nucleoplasm and was localized to the chromosome surface during mitosis (Fujimoto et al. (2004)). The At-hook of this factor was shown to be necessary for binding of the matrix attachment region (MAR). Such a result suggests that AHL1 (G1944) has a role in regulating chromosome dynamics, or protection of the chromosomes during cell division. G1944 is relatively distantly related to G1073 and lies outside of the G1073 clade. However, the result is of interest as it evidences the fact the single At-hook class proteins as well as the HMGA class (which have multiple At-hooks) can have structural roles in organizing chromosomes.

Overexpression of G1073 in Arabidopsis. We established that overexpression of G1073 leads to increased vegetative biomass and seed yield compared to control plants. As a result of these phenotypes we assigned the gene name HERCULES1 (HRC1) to G1073. Drought tolerance was observed in 35S::G1073 transgenic lines. More recently we observed hyperosmotic stress-tolerance phenotypes, such as tolerance to high salt and high sucrose concentrations, in plate assays performed on 35S::G1073 plants.

35S::G1073 Arabidopsis lines display enlarged organs, due to increased cell size and number. We also conducted some preliminary analyses into the basis of the enhanced biomass of 35S::G1073 Arabidopsis lines. We found that the increased mass of 35S::G1073 transgenic plants could be attributed to enlargement of multiple organ types including leaves, stems, roots and floral organs. Petal size in the 35S::G1073 lines was increased by 40-50% compared to wild type controls. Petal epidermal cells in those same lines were approximately 25-30% larger than those of the control plants. Furthermore, we found 15-20% more epidermal cells per petal, compared to wild type. Thus, at least in petals, the increase in size was associated with an increase in cell size as well as in cell number. Additionally, images from the stem cross-sections of 35S::G1073 plants revealed that cortical cells were large and that vascular bundles contained more cells in the phloem and xylem relative to wild type.

To quantify the 35S::G1073 phenotype we examined the fresh and dry weight of the plants (Table 1). 35S::G1073 lines showed an increase of at least 60% in biomass. More importantly, the 35S::G1073 lines showed an increase of at least 70% in seed yield. This increased seed production appears to be associated with an increased number of siliques per plant, rather than seeds per silique or increased size.

TABLE 1
Comparison of wild type and G1073 overexpressor
biomass and seed yield production

| Line | Fresh weight (g) | Dry weight (g) | Seed (g) |
|---|---|---|---|
| WT | 3.43 ± 0.70 | 0.73 ± 0.20 | 0.17 ± 0.07 |
| 35S::G1073-3 | 5.74 ± 1.74 | 1.17 ± 0.30 | 0.31 ± 0.08 |
| 35S::G1073-4 | 6.54 ± 2.19 | 1.38 ± 0.44 | 0.35 ± 0.12 |

Average value (±standard error) from 20 plants harvested at near end of life cycles (70 days after planting)

Genetic regulation of organ size in plants. To use G1073 in the engineering of drought tolerance, without incurring increased organ size phenotypes, an understanding of the genetic control features of organ size is necessary. Organ size is under genetic control in both animals and plants, although the genetic mechanisms of control are likely quite distinct between these kingdoms. Current understanding of organ size control in plants is limited, but what is known has been summarized by Hu et al. (2003); Krizek (1999); Mizukama and Fischer (2000); Lincoln et al. (1990); Zhong and Ye (2001); Ecker (1995); Nath et al. (2003); and Palatnik et al. (2003). Organ size is regulated by both external and internal factors, with a general understanding that these factors contribute to the maintenance of meristematic competence. The “organ size control checkpoint”, which is thought to regulate meristematic competence, is the determining feature in the control of organ size (Mizukami (2001)). There are a few genes that have been shown previously to contribute to organ size control, including AINTEGUMENTA (Krizek (1999); Mizukami and Fischer (2000)), AXR1 (Lincoln et al. (1990)), and ARGOS (Hu et al. (2003)). Not surprisingly, these genes are involved with hormone response pathways, particularly auxin response pathways. For example, ARGOS was identified initially through microarray experiments as being highly up-regulated by auxin. ARGOS was subsequently shown to increase organ size when overexpressed in Arabidopsis (Hu et al. (2003)). Additionally, a number of publications have implicated proteins from the TCP family in the control of organ size and shape in Arabidopsis (Cubas et al. (1999); Nath et al. (2003); Palatnik et al. (2003); Crawford et al. (2004)).

We have begun to examine how the pathways through which G1073 acts related to the known pathways of organ growth regulation. In particular, we are investigating the idea that G1073 regulates a pathway that regulates organ growth in response to environmentally derived stress signals.

### Background Information for G682, the G682 Clade, and Related Sequences

We identified G682, SEQ ID NO: 60, as a transcription factor from the Arabidopsis BAC AF007269 based on sequence similarity to other members of the MYB-related family within the conserved domain. To date, no functional data are available for this gene in the literature. The gene corresponds to At4G01060, annotated by the Arabidopsis Genome initiative. G682 is member of a clade of related proteins that range in size from 75 to 112 amino acids. These proteins contain a single MYB repeat, which is not uncommon for plant MYB transcription factors. Information on gene function has been published for four of the genes in this clade, CAPRICE (CPC/G225), TRIPTYCHON (TRY/G1816), ENHANCER of TRY and CPC 1 (ETC1/G2718) and ENHANCER of TRY and CPC 2 (ETC2/G226). Published information on gene function is not available for G682, or for G3930 (SEQ ID NO: 411) which was only recently identified. The G3930 locus has not been recognized in the public genome annotation. Members of the G682 clade were found to promote epidermal cell type alterations when overexpressed in Arabidopsis. These changes include both increased numbers of root hairs compared to wild type plants, as well as a reduction in trichome number. In addition, overexpression lines for the first five members of the clade showed a reduction in anthocyanin accumulation in response to stress, and enhanced tolerance to hyperosmotic stress. In the case of 35S::G682 transgenic lines, an enhanced tolerance to high heat conditions was also observed. Given the phenotypic responses for G682 and its clade members, all members of the clade were included in our studies. The analysis of G225 (CPC), however, has been limited. Table 2 summarizes the functional genomics program data on G682 and its clade members.

TABLE 2
G682-clade traits

| | CPC | G226 (SEQ | G682 (SEQ | TRY (G1816, | G2718 (SEQ |
|---|---|---|---|---|---|
| | (G225) | ID NO: 62) | ID NO: 60) | SEQ ID NO: 76) | ID NO: 64) |
| Reduction in Trichome # | X | X | X | X | X |
| Increased Root Hair # | X | X | X | X | X |
| N Tolerance | X | X | | X | X |
| Heat Tolerance | X | | X | | |
| Salt Tolerance | | X | | | |
| Sugar response | | | | X | |

MYB (Myeloblastosis) transcription factors. MYB proteins are functionally diverse transcription factors found in both plants and animals. They share a signature DNA-binding domain of approximately 50 amino acids that contains a series of highly conserved residues with a characteristic spacing (Graf (1992)). Critical in the formation of the tertiary structure of the conserved Myb motif is a series of consistently spaced tryptophan residues (Frampton et al. (1991)). Animal Mybs contain three repeats of the Myb domain: R1, R2, and R3. Plant Mybs usually contain two imperfect Myb repeats near their amino termini (R2 and R3), although there is a small subgroup of three repeat (R1R2R3) mybs similar to those found in animals, numbering approximately eight in the Arabidopsis genome. A subset of plant Myb-related proteins contain only one repeat (Martin and Paz-Ares (1997)). Each Myb repeat has the potential to form three alpha-helical segments, resembling a helix-turn-helix structure (Frampton et al. (1991)). Although plant Myb proteins share a homologous Myb domain, differences in the overall context of their Myb domain and in the specific residues that contact the DNA produce distinct DNA-binding specificities in different members of the family. Once bound, MYB proteins function to facilitate transcriptional activation or repression, and this sometimes involves interaction with a protein partner (Goff et al. (1992)). We divide MYB transcription factors into two families; the MYB (R1)R2R3 family which contains transcription factors that typically have two imperfect MYB repeats, and the MYB-related family which contains transcription factors that contain a single MYB-DNA binding motif.

The MYB-related family (Single-repeat MYB transcription factors). There are approximately 50 members of this family in Arabidopsis. The MYB-related DNA-binding domain contains approximately 50 amino acids with a series of highly conserved residues arranged with a characteristic spacing. The single-repeat MYB proteins do not contain a typical transcriptional activation domain and this suggests that they may function by interfering with the formation or activity of transcription factors or transcription factor complexes (Wada et al. (1997); Schellmann et al. (2002)). In addition to the G682 clade, two well characterized transcription factors, CIRCADIAN CLOCK ASSOCIATED1 (CCA1/G214/SEQ ID NO: 345) and LATE ELONGATED HYPOCOTYL (LHY/G680/SEQ ID NO: 343) represents additional well-characterized MYB-related proteins that contain single MYB repeats (Wang et al. (1997); Schaffer et al. (1998)).

Epidermal cell-type specification. Root hair formation and trichome formation are two processes that involve the G682 clade members. Epidermal cell fate specification in the Arabidopsis root and shoot involves similar sets of transcription factors that presumably function in mechanistically similar ways (Larkin et al. (2003)). The initial step in cell-type specification in both cases is evidently controlled by antagonistic interactions between G682-clade members and other sets of genes (Table 3). In the case of the shoot epidermis, G682 clade members repress trichome specification, and in the case of the root epidermis G682 clade members promote root-hair specification. Table 4 compiles the list of genes that have been implicated in root hair and trichome cell specification through genetic and biochemical characterization where both loss-of-function and gain-of-function phenotypes have been analyzed. The specific roles of these genes are discussed in the following sections.

TABLE 3
Antagonistic interactions in epidermal cell-type specification.

| | Root Hair Fate | Trichome Fate |
|---|---|---|
| Promotes | CPC/TRY (G682 clade) | GL1 (R2R3 MYB), TTG |
| | | (WD-repeat), GL3 (bHLH) |
| Represses | WER(R2R3 MYB), TTG | CPC/TRY (G682 clade) |
| | (WD-repeat), GL3 (bHLH) | |

TABLE 4
Transcription factors involved in epidermal cell fate

| Gene | GL3 | EGL3 | GL1 | WER | GL2 | TTG1 | CPC | TRY | ETC1 | ETC2 |
|---|---|---|---|---|---|---|---|---|---|---|
| Name | | | | | | | | | | |
| GID | G585 | G581 | G212 | G676 | G388 | n/a | G225 | G1816 | G2718 | G226 |
| SEQ ID | 340 | 338 | 348 | 350 | 352 | | | 76 | 64 | 62 |
| NO. | | | | | | | | | | |
| Gene | bHLH/ | bHLH/ | MYB- | MYB- | HD | n/a | MYB- | MYB- | MYB- | MYB |
| Family | MYC | MYC | (R1) | (R1) | | | related | related | related | related |
| | | | R2R3 | R2R3 | | | | | | |
| Paralogs | G586 | | G247, | G212, | none | n/a | G226, | G225, | G225, | G225, |
| | | | G676 | G247 | | | G682, | G226, | G226, | G682, |
| | | | | | | | G1816, | G682, | G682, | G1816, |
| | | | | | | | G2718, | G2718, | G1816, | G2718, |
| | | | | | | | G3930 | G3930 | G3930 | G3930 |
| Loss-of- | Slight root | Slight root | Glabrous | All cell | Ectopic | All cell | No root | wild-type | wild-type | wild-type |
| Function | hair | hair | | files are | hairs, | files are | hairs, | roots, | roots and | roots, |
| | increase, | increase, | | hairs | glabrous | hairs | ectopic | ectopic tri- | shoots | ectopic |
| | reduction | reduction | | | | glabrous | tri- | chomes | | tri- |
| | in | in | | | | | chomes | | | chomes |
| | trichome | trichome | | | | | | | | |
| | number | number | | | | | | | | |
| Gain-of- | Ectopic | Ectopic | Ectopic | Wild- | Wild- | Wild-type | Ectopic | Ectopic | Ectopic | Ectopic |
| Function | trichomes | tri-chomes | tri- | type | type | | root hairs, | root hairs, | root hairs, | root hairs, |
| | | | chomes | | | | glabrous | glabrous | glabrous | glabrous |
| Site of | Leaf and | Leaf, | Leaf | Root | Leaf Epidermis, | Leaf Epidermis, | Leaf | Leaf Epidermis | Leaf | Leaf |
| Activity | root | Root | epidermis | Epidermis | Root Epidermis | Root | Epidermis | and | Epidermis | Epidermis |
| | epidermis | Epidermis, | | | and Seed | Epidermis | and | Root | and | and |
| | | Seed Coat | | | Coat | and | Root | Epidermis | Root | Root |
| | | | | | | Seed Coat | Epidermis | | Epidermis | Epidermis |
| Citations | 1, 2 | 2 | 3 | 4 | 3, 5 | 6 | 7 | 8 | 9 | 10 |

References:
(1) Payne et al. (2000);
(2) Zhang et al. (2003);
(3) Di Cristina et al. (1996);
(4) Lee and Schiefelbein (1999);
(5) Masucci J. et al. (1996);
(6) Galway et al. (1994);
(7) Wada et al. (1997);
(8) Schellmann et al. (2002);
(9) Kirik et al. (2004a);
(10) Kirik et al. (2004b)

Leaf epidermis cell-type specification: GLABRA2 (GL2/G388) encodes a homeodomain-leucine zipper protein that promotes non-hair cell fate in roots and trichome fate in the shoot; and GL2 expression represents a critical regulatory step in the process of epidermal cell-type differentiation in both the root and shoot. In leaf epidermal tissue, the default program is the formation of a trichome cell which is promoted by GL2 expression. GL2 is induced by a proposed “activator complex” that is composed of GL1 (G212), an R2R3MYB protein, TTG1 a WD-40 repeat containing protein, and GL3 (G585) a bHLH transcription factor. The formation of this complex is supported by genetic data as well as by biochemical data (Larkin et al. (2003)). Yeast 2-hybrid data shows that GL3 interacts directly with both TTG1 and GL1 (Payne et al. (2000)). Non-trichome cell fate, on the other hand, is specified in neighboring cells through the combined activity of TRY (G1816), CPC (G225), ETC1 (G2718) and ETC2 (G226), which are all members of the G682 clade. In this report, we determined the expression pattern of G682 throughout development, to compare with expression patterns from other clade members. Since 35S::G682 lines are glabrous, G682 is also likely to participate in the suppression of trichome fate in the epidermis of wild-type leaves. The precise mechanism by which each clade member acts is, however, unknown. Later in organ development, TRY (G1816), CPC (G225), ETC1 (G2718) and ETC2 (G226) are expressed at relatively high levels in trichomes (Schellmann et al. (2002); Kirik et al. (2004a); Kirik et al. (2004b)), whereas there is no published expression data on G682.

One intriguing result related to the expression of both CPC and TRY is that they are not expressed preferentially in the cells adjacent to the trichomes where they act to suppress trichome fate. In fact, CPC and TRY transcription is induced by GL1 in cells that become trichomes. Schellmann et al. (2002), have proposed a “lateral inhibition” model to explain this paradox. Lateral inhibition is a process whereby a cell that is taking a certain fate prevents its neighbors from taking that same fate. The mechanism of lateral inhibition involves diffusible activators and repressors, and the activator complex stimulates its own expression as well as that of the repressor. The repressor then moves across cell boundaries to suppress the activator complex found in neighboring cells.

GL1, TTG1 and GL3 function in a regulatory feedback loop, enhancing their own expression. A complex composed of those three proteins activates GL2 which promotes trichome cell fate. The GL1/TTG/GL3 complex also serves to activate the repressors CPC and TRY which suppress their expression, and trichome formation, in neighboring cells. The repressors (CPC/TRY) are proposed to move across the cell boundary resulting in the suppression of the activator complex in neighboring cells. In other words, in cells where the proteins are initially being produced, the scales are still tipped in the direction of the activator and in the neighboring cells the scales are tipped in the direction of the repressor. It is worth noting that a CPC:GFP fusion protein has been shown to move from cell to cell in the epidermis of the root (Wada et. al. (2002)), presumably through plasmodesmata.

Root epidermis cell-type specification: In the root epidermis the “activator complex” and GL2 promote non-hair cell fate, and in neighboring cells CPC and TRY (as well as ETC1 and ETC2) promote root hair fate. Involvement of CPC in a lateral inhibition model in root hair cell specification was supported by a series of genetic experiments described recently by Lee and Schiefelbein (2002). The proposed “activator” that is important for the specification of a non-root hair cell fate is thought to be composed of WER (G676; a MYB-related transcription factor and paralog to GL1), TTG and GL3. Recently, Zhang et al. (Zhang et al. (2003)) published results confirming the function of GL3 in root epidermal specification, and they identified a second bHLH transcription factor EGL3 (G581) that also presumably can function in the “activator complex”. EGL3 (G581) overexpressors showed increased tolerance to low nitrogen conditions in our earlier Arabidopsis functional genomics program G581, SEQ ID NO: 338, also had a seed anthocyanin phenotype when overexpressed. The repressor proteins in this model are, again, CPC and TRY (along with ETC1 and ETC2; Kirik et al. (2004a) and Kirik et al. (2004b)). Consistent with this model, Lee and Schiefelbein (2002) have shown that CPC inhibits the expression of WER, GL2 and itself. They have also shown that WER activates GL2 and CPC. As mentioned above CPC:GFP fusion proteins move from cell to cell in the root epidermis (Wada et al. (2002)), and it is known that specification begins prior to significant cell expansion (Costa and Dolan (2003)) at a time when the root epidermis is symplastically contiguous (Duckett et al. (1994)).

One striking feature of root hair specification is that the root hairs are always placed over the end-wall of the underlying cortical cells. This highly consistent placement of trichomes strongly suggests that the epidermal cells are responding to cues from below. Here we suggest two hypotheses for how signals from beneath the epidermis pre-pattern it. In the first hypothesis, an apoplastic signal moves between the cortex cells and promotes a bias towards CPC/TRY in the epidermal cells that contact the wall. Ethylene is one candidate for such an apoplastic signal, and ethylene is known to affect root hair differentiation in Arabidopsis (Taminoto et al. (1995); Di Cristina et al. (1996)).

In the second hypothesis, a polarity in the cortical cells with regard to cortex-to-epidermis signaling could pre-pattern the epidermis. It is worth noting that CPC is expressed in all cell layers of the root in the region of specification (Wada et al. (2002); Costa and Dolan (2003); thus it is possible that CPC/TRY moves into theepidermis from the cortical cell layer. The preferential transport of CPC/TRY near the side-wall of the cortical cells could lead to a CPC/TRY bias in the cells that contact two cortical cells (i.e., the cells that are specified as hair cells). Alternatively, the differential movement of unknown symplastic signals could also act to pre-pattern the epidermis.

A receptor-like kinase, SCRAMBLED (SCM, which disrupts the precise striped patterning of epidermal cell files in Arabidopsis, has recently been identified (Kwak et al. (2005)). In scm mutants, epidermal patterning genes such as WER and GL2 are no longer expressed in long cell files, but instead are expressed in a patchy manner. The specification of root hair and non-hair cells also occurs in a patchy manner. Although SCM is evidently required for proper cell-file patterning, it is unclear precisely how it fits into specification processes. The expression of this gene is not specific to either hair cells, or non-hair cells, and thus SCM is unlikely to be sufficient for establishing cell-type identity. At present, no ligand for SCM has been identified. Curiously, the expression of SCM is relatively low in the epidermis, and much higher in the cell-layers underlying it (Kwak et al. (2005)). The significance, if any, of the high levels of expression in inner cell layers is not known.

Discoveries made in earlier genomics programs. The difference in the phenotypic responses of the G682-clade overexpression lines (Table 2), along with the differences in the CPC (G225) and TRY (G1816) mutant phenotypes (Schellmann et al. (2002)), suggest that each of the 5 genes in the clade have distinct but overlapping functions in the plant. In the case of 35S::G682 transgenic lines, an enhanced tolerance to high heat conditions was observed. Heat can cause osmotic stress, and it is therefore reasonable that these transgenic lines were also more tolerant to drought stress in a soil-based assay. Another common feature for 4 of the members of this clade is that they enhance performance under nitrogen-limiting conditions. 35S::G682 plants were not identified as having enhanced performance under nitrogen-limiting conditions in the genomics program. We have evaluated, in this report, performance of G682 and its clade members with respect to various assays suggesting altered nitrogen utilization.

All of the genes in the Arabidopsis G682 clade reduced trichomes and increased root hairs when constitutively overexpressed (Table 2). It is unknown, however, whether the drought-tolerance phenotype in these lines is related to the increase in root hairs on the root epidermis. Increasing root hair density may increase in absorptive surface area and increase in nitrate transporters that are normally found there. Alternatively, the wer, ttg1 and gl2 mutations, all of which increase root hair frequency, and have also been shown to cause ectopic stomate formation on the epidermis of hypocotyls. Thus, it is possible that the G682 clade could be involved in the development, or regulation, of stomates (Hung et al. (1998); Berger et al. (1998); Lee and Schiefelbein (1999)). The CPC (G225) and TRY (G1816) proteins have not been reported to alter hypocotyl epidermal cell fate, however; the role of G682 in stomatal guard cell density is evaluated in this report. Alterations in stomate function could also alter plant water status, and guard-cell apertures and light response remain to be examined in G682-clade overexpression lines.

Interestingly, our data also suggest that G1816 (TRY) overexpression lines have a glucose sugar sensing phenotype. Several sugar sensing mutants have turned out to be allelic to ABA and ethylene mutants. This potentially implicates G1816 in hormone signaling and in an interaction of hormone signaling, stress responses and sugars.

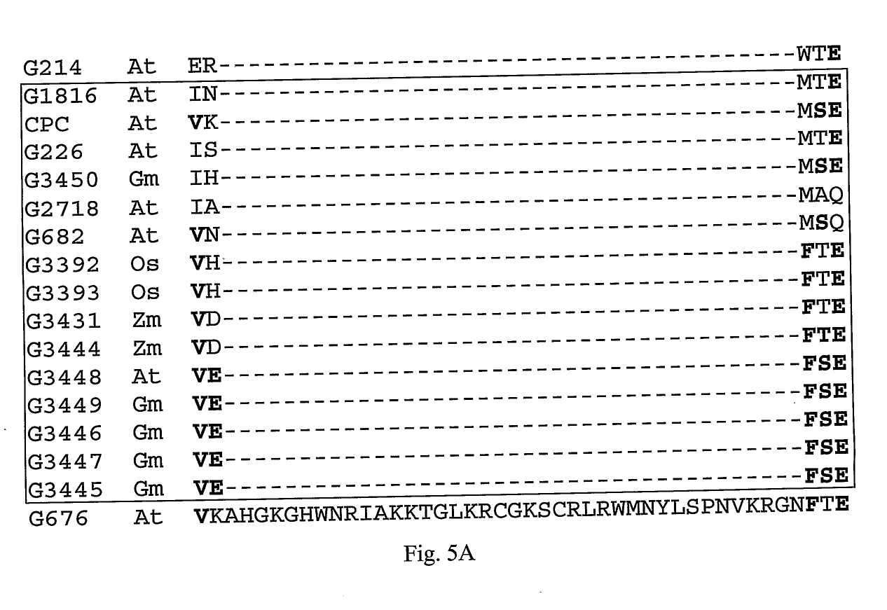

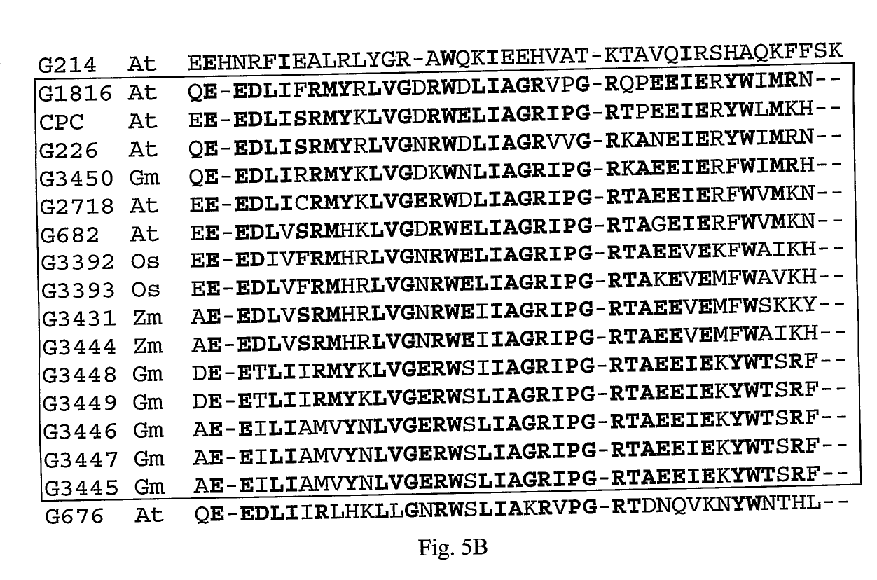

Protein structure and properties. G682 and its paralogs and orthologs are composed (almost entirely) of a single MYB-repeat DNA binding domain that is highly conserved across plant species. An alignment of the G682-like proteins from Arabidopsis, soybean, rice and corn that are being analyzed is shown in FIGS. 5A and 5B.

Because the G682 clade members are short proteins that are comprised almost exclusively of a DNA binding motif, it is likely that they function as repressors. This is consistent with in expression analyses indicating that CPC represses its own transcription as well as that of WER and GL2 (Wada et al. (2002); Lee and Schiefelbein (2002)). Repression may occur at the level of DNA binding through competition with other factors at target promoters, although repression via protein-protein interactions cannot be excluded.

We first identified G867, SEQ ID NO: 88, as a transcription factor encoded by public EST sequence (GenBank accession N37218). Kagaya et al. (Kagaya et al. (1999)) later assigned the gene the name Related to ABI3/VP1 1 (RAV1) based on the presence of a B3 domain in the C-terminal portion of the encoded protein. In addition to the B3 domain, G867 contains a second DNA binding region, an AP2 domain, at its N terminus. There are a total of six RAV related proteins with this type of structural organization in the Arabidopsis genome: G867 (AT1G13260, RAV1), G9 (AT1G68840, which has been referenced as both RAP2.8, Okamuro et al. (1997), and as RAV2, Kagaya et al. (1999)), G1930, SEQ ID NO: 92 (AT3G25730), G993, SEQ ID NO: 90 (AT1G25560), G2687, SEQ ID NO: 380 (AT1G50680), and G2690, SEQ ID NO: 382 (AT1G51120). Recently, G867 was identified by microarray as one of 53 genes down-regulated by brassinosteroids in a det2 (BR-deficient) cell culture. This down-regulation was not dependent on BRI1, and mild down-regulation of G867 also occurred in response to cytokinins (Hu et al. (2004). These authors also showed that overexpression of G867 reduces both root and leaf growth, and causes a delay in flowering. A G867 knockout displays early flowering time, but no other obvious effect. A detailed genetic characterization has not been published for any of the other related genes.

On the basis of the AP2 domain, the six RAV-like proteins were categorized as part of the AP2 family. However, the B3 domain is characteristic of proteins related to ABI3/VP1 (Suzuki et al. (1997)).

AP2 domain transcription factors. The RAV-like proteins form a small subgroup within the AP2/ERF family; this large transcription factor gene family includes 145 transcription factors (Weigel (1995); Okamuro et al. (1997); Riechmann and Meyerowitz (1998); Riechmann et al. (2000a). Based on the results of the our genomics screens it is clear that this family of proteins affect the regulation of a wide range of morphological and physiological processes, including the acquisition of stress tolerance. The AP2 family can be further divided into three subfamilies:

The APETALA2 class is related to the APETALA2 protein itself (Jofuku et al. (1994)), characterized by the presence of two AP2 DNA binding domains, and contains 14 genes.

The AP2/ERF is the largest subfamily, and includes 125 genes, many of which are involved in abiotic (DREB subgroup) and biotic (ERF subgroup) stress responses (Ohme-Takagi and Shinshi (1995); Zhou et al. (1995b) Stockinger et al. (1997); Jaglo-Ottosen et al. (1998); Finkelstein et al. (1998)).

The 6 genes from the RAV subgroup, all of which have a B3 DNA binding domain in addition to the AP2 DNA binding domain.

B3 domain transcription factors. ABI3/VP1 related genes have been generally implicated in seed maturation processes. The ABSCISIC ACID INSENSITIVE (ABI3, G621, SEQ ID NO: 376) protein and its maize ortholog VIVIPAROUS1 (VP1) regulate seed development and dormancy in response to ABA (McCarty et al. (1991); Giraudat et al. (1992)). ABI3 (G621, SEQ ID NO: 376) and VP1 play an important role in the acquisition of desiccation tolerance in late embryogenesis. This process is related to dehydration tolerance as evidenced by the protective function of late embryogenesis abundant (LEA) genes such as HVA1 (Xu et al. (1996), Sivamani et al. (2000)). Mutants for Arabidopsis ABI3 (Ooms et al. (1993)) and the maize ortholog VP1 (Carson et al. (1997), and references therein) show severe defects in the attainment of seed desiccation tolerance. ABI3 activity is normally restricted to the seeds. However, overexpression of ABI3 from a 35S promoter was found to increase ABA levels, induce several ABA/cold/drought-responsive genes such as RAB18 and RD29A and increased freezing tolerance in Arabidopsis (Tamminen et al. (2001)). These data illustrate the relatedness of the processes of seed desiccation and dehydration tolerance and demonstrates that the seed-specific ABI3 transcription factor does not require additional seed-specification proteins to function vegetative tissues. Recently, a tight coupling has been demonstrated between ABA signaling and ABI3/VP1 function; Suzuki et al. (Suzuki et al. (2003)) found that the global gene expression patterns caused by VP1 overexpression in Arabidopsis were very similar to patterns produced by ABA treatments.

Regulation by ABI3/VP1 is complex: the protein is a multidomain transcription factor that can apparently function as either an activator or a repressor depending on the promoter context (McCarty et al. (1991); Hattori et al. (1992); Hoecker et al. (1995); Nambara et al. (1995)). In addition to the B3 domain, ABI3/VP1 has two other protein domains (the B1 and B2 domains) that are also highly conserved among ABI3/VP1 factors from various plant species (McCarty et al. (1991)). Targets of the different domains have been identified. Both in Arabidopsis and maize, the B3 domain of ABI3/VP1 binds the RY/SPH motif (Ezcurra et al. (2000)); Carson et al. (1997)), whereas the N terminal B1 and B2 domains are implicated in nuclear localization and interactions with other proteins. In particular, the B2 domain is thought to act via ABA response elements (ABREs) in target promoters. VP1 has been shown to activate ABREs through a core ACGT motif (called the G-Box), but does not bind the element directly. However, a number of bZIP transcription factors have been shown to bind ABREs in the promoters of ABA induced genes (Guiltinan et al. (1990); Jakoby et al. (2002)), and recent data suggest that VP1 might induce ABREs via interactions with these bZIP proteins. Such evidence was afforded by Hobo et al. (1999) who demonstrated interaction between the rice VP1 protein OsVP1 and a rice bZIP protein, TRAB1. While in Arabidopsis the B3 domain of ABI3 is essential for abscisic acid dependent activation of late embryogenesis genes (Ezcurra et al. (2000)), the B3 domain of VP1 is not essential for ABA regulated gene expression in maize seed (Carson et al. (1997), McCarty et al. (1989)), though the B3 domain of G9 RAV2, is able to act as an ABA agonist in maize protoplasts (Gampala et al. (2004)). The difference in the regulatory network between Arabidopsis and maize can be explained by differential usage of the RY/SPH versus the ABRE element in the control of seed maturation gene expression (Ezcurra et al. (2000)). The RY/SPH element is a key element in gene regulation during late embryogenesis in Arabidopsis (Reidt et al. (2000)) while it seems to be less important for seed maturation in maize (McCarty et al. (1989)).

Similarity to the B3 domain has been found in several other plant proteins, including the Arabidopsis FUSCA3 (FUS3, G1014, SEQ ID NO: 378). The FUS3 protein can be considered as a natural truncation of the ABI3 protein (Luerssen et al. (1998)); like ABI3, FUS3 binds to the RY/SPH element, and can activate expression from target promoters even in non-seed tissues (Reidt et al. (2000)). ABI3 domain is also present in LEAFY COTYLEDON 2 (Luerssen et al. (1998); Stone et al. (2001)). ABI3, FUS3, LEC2 (G3035, SEQ ID NO: 384), and LEAFY COTYLEDON 1 are known to act together to regulate many aspects of seed maturation (Parcy et al. (1997); Parcy and Giraudat (1997); Wobus and Weber (1999)). (LEC1, G620, SEQ ID NO: 358, is a CAAT box binding transcription factor of the HAP3 class, Lotan et al. (1998)). Like abi3 mutants, mutants for these other three genes also show defects in embryo specific programs and have pleiotropic phenotypes, including precocious germination and development of leaf like characters on the cotyledons. Unlike abi3, though, these mutants have almost normal ABA sensitivity and are not directly implicated in ABA signaling (Meinke (1992); Keith et al. (1994); Meinke et al. (1994)). Overexpression of either LEC1 or LEC2 results in ectopic embryo formation (Lotan et al. (1998); Stone et al. (2001)), supporting the role of this gene in the regulation of embryo development.

Although the ABI3 related genes containing a B3 domain have roles related to abiotic stress tolerance during embryo maturation, it remains to be reported whether all proteins containing a B3 domain have a general role in such responses or in embryo development. Detailed genetic analyses have not been published on the RAV genes; however, RAV1 has been implicated in abiotic stress responses based on the observation that it is transcription up-regulated on cold acclimation (Fowler and Thomashow (2002)). A similar result was seen the RT-PCR studies performed during our initial genomics program, when we found that G867 was up-regulated by cold or auxin treatments. We also found that the G867 paralog, G1930, SEQ ID NO: 92, was up-regulated by cold or auxin treatments.

It is particularly intriguing that G867 expression was induced by auxin treatment, since transcription factors from the auxin response factor (ARF) class also contain a B3 related domain and respond to auxin (Uimasov et al. (1997)). ARF transcription factors only contain a single DNA binding domain. However, the current models predict that ARFs generally function as dimers (Liscum and Reed (2002)). It is unknown whether G867 could interact with ARF proteins. It has been shown that a G867 monomer is sufficient for DNA binding, yet this does not exclude potential interactions with other proteins.

Discoveries made in earlier genomics programs. G867 was included based on the enhanced tolerance of 35S::G867 lines to drought related hyperosmotic stresses such as sucrose and salt. Further testing revealed a moderate increase in drought tolerance in a soil based assay, which finally triggered the inclusion in the program.

Following our initial discovery of G867 in the form of a public EST (GenBank accession N37218) we first examined the function of the gene using a homozygous line that contained a T-DNA insertion immediately downstream of the G867 conserved AP2 domain. This insertion would have been expected to result in a severe or null mutation. However, the KO.G867 plants did not show significant changes in morphological and physiological analyses compared to wild-type controls, suggesting that the gene might have a redundant role with one or more of the other three RAV genes.

Subsequently, we assessed the function of G867 using 35S::G867 lines; in these assays, most of these lines were recorded as showing no consistent morphological differences to wild type. However, the plants exhibited increased seedling vigor (manifested by increased expansion of the cotyledons) in germination assays on both high salt and high sucrose media, compared to wild-type controls. Overexpression lines for the Arabidopsis paralogs of G867, G1930 and G9, also exhibited stress-related phenotypes, suggesting a general involvement of this clade in abiotic stress responses. 35S::G9 plants also showed increased root biomass and 35S::G1930 lines exhibited tolerance to high salt and sucrose (this phenotype was identical to that seen in 35S::G867 lines). Overexpression lines for the final paralog, G993, SEQ ID NO: 90, however, did not show a significant difference to wild type in our initial physiological assays. However, 35S::G993 seedlings had a variety of developmental defects, and the plants produced seeds, which were pale in coloration, suggesting that the gene might influence seed development.

Protein structure and properties. G867 lacks introns and encodes a 344 amino acid protein with a predicted molecular weight of 38.6 kDa. Analysis of the binding characteristics of RAV1 (G867) revealed that the protein binds as a monomer to a bipartite target consisting of a CAACA and a CACCTG motif which can be separated by 2-8 nucleotides, and can be present in different relative orientations (Kagaya et al. (1999)). Gel shift analysis using different deletion variants of RAV1 have shown that the AP2 domain recognizes the CAACA motif while the B3 domain interacts with the CACCTG sequence. Although both binding domains function autonomously, the affinity for the target DNA is greatly enhanced when both domains are present (Kagaya et al. (1999)), suggesting that the target DNA can act as an allosteric effector (Lefstin and Yamamoto (1998)).

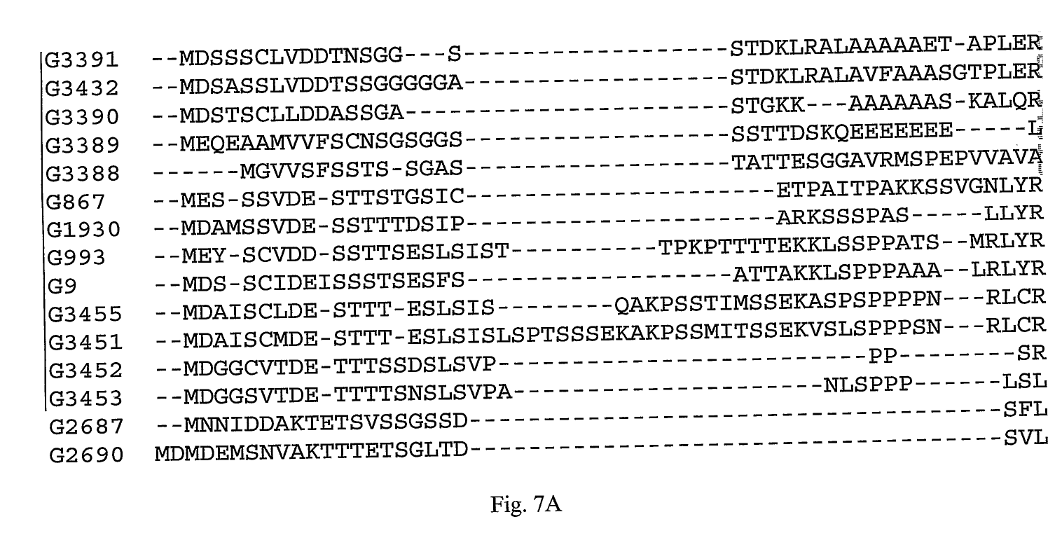

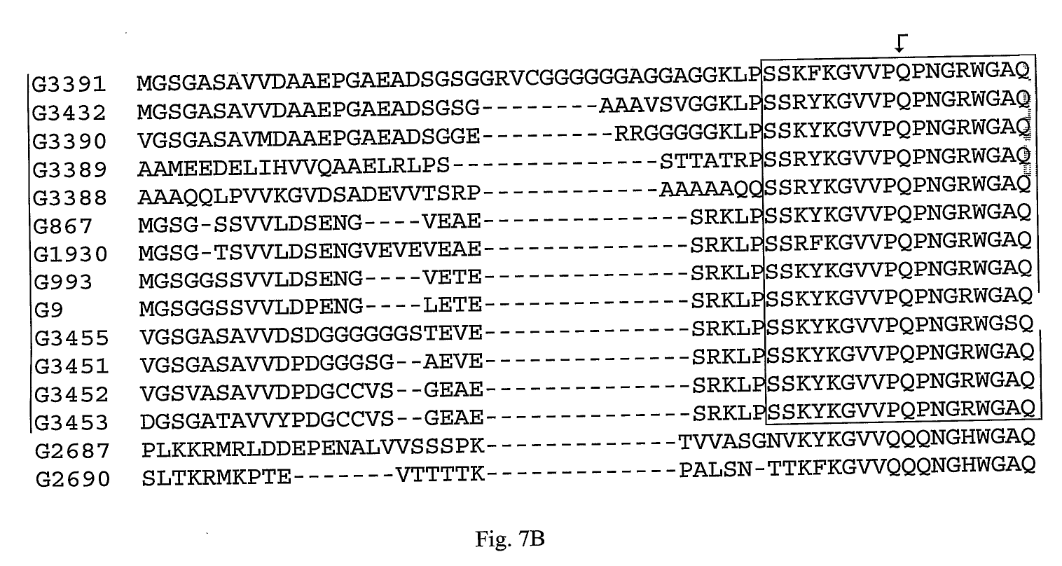

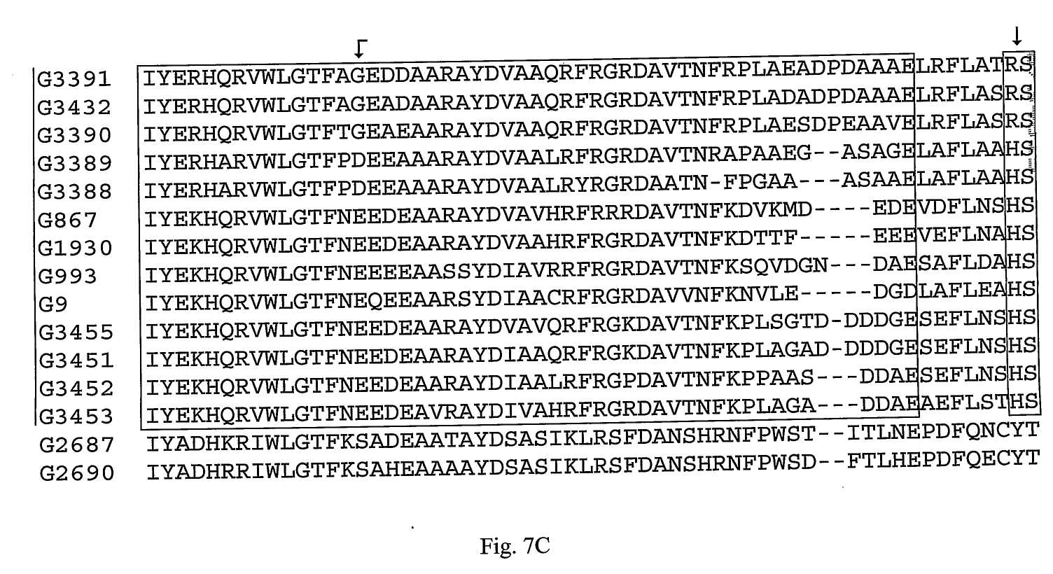

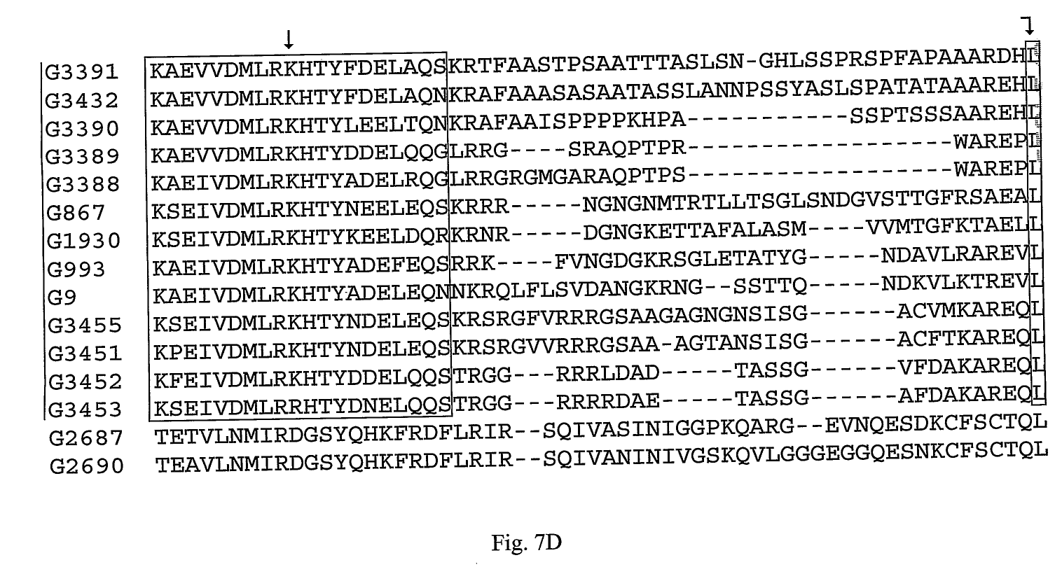

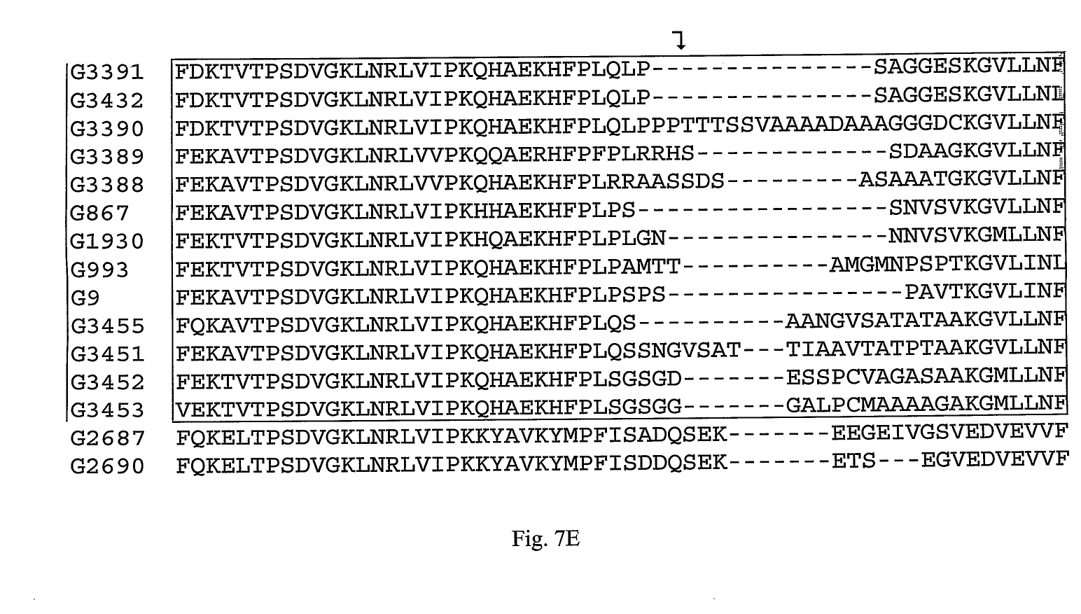

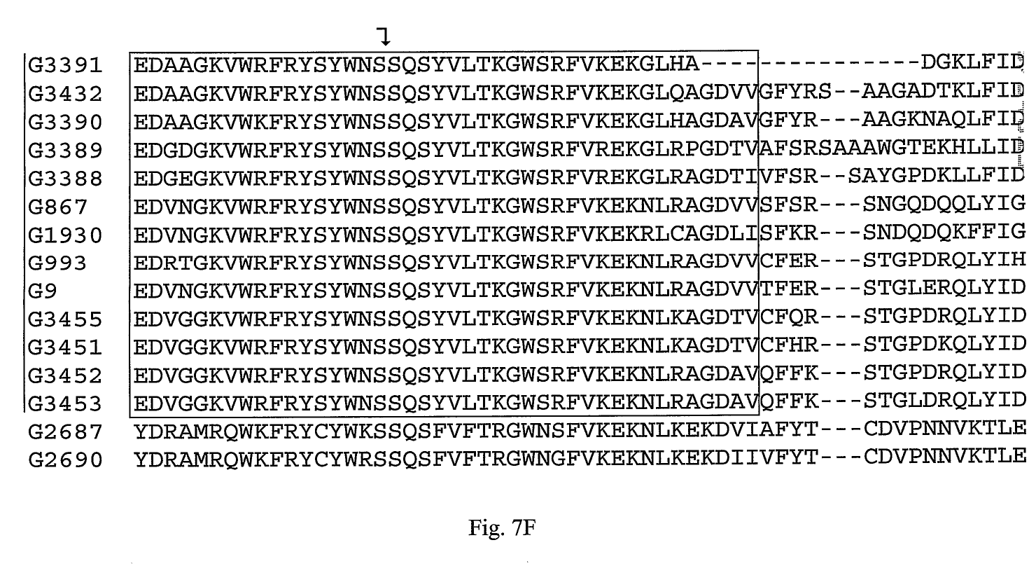

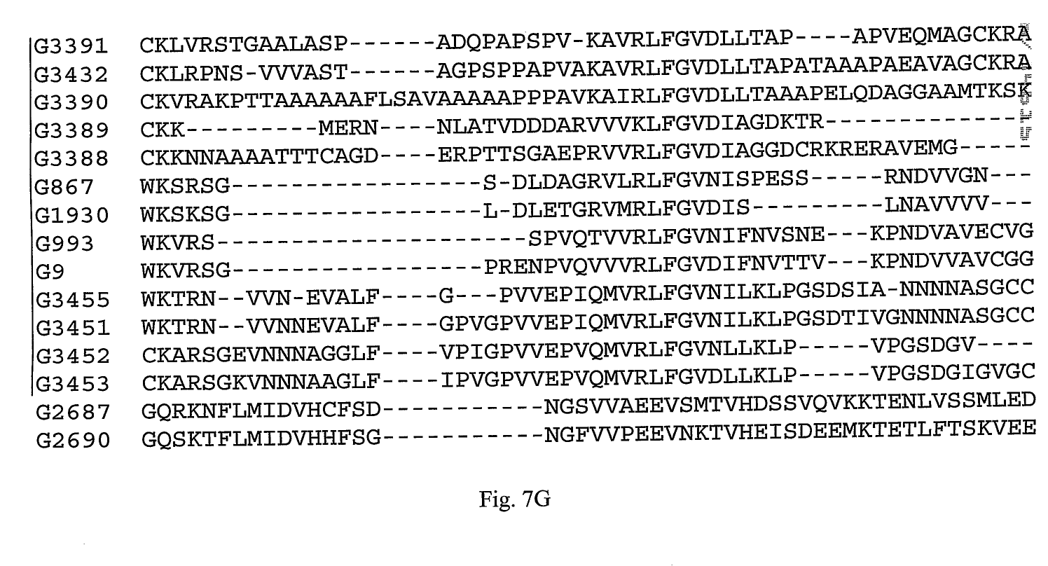

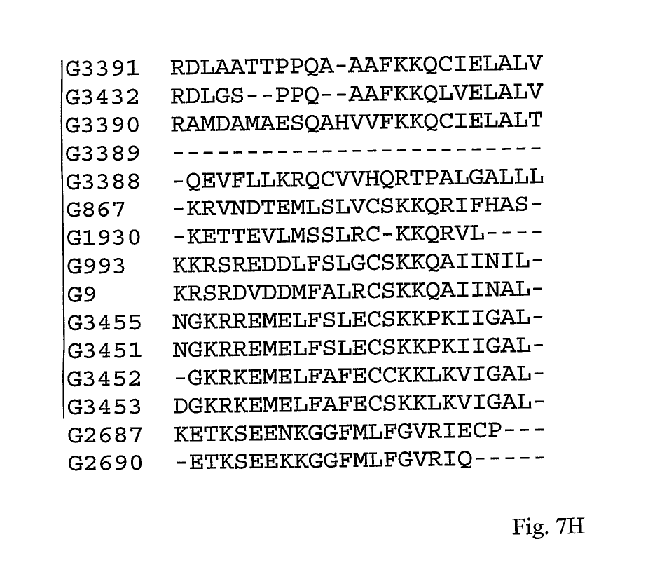

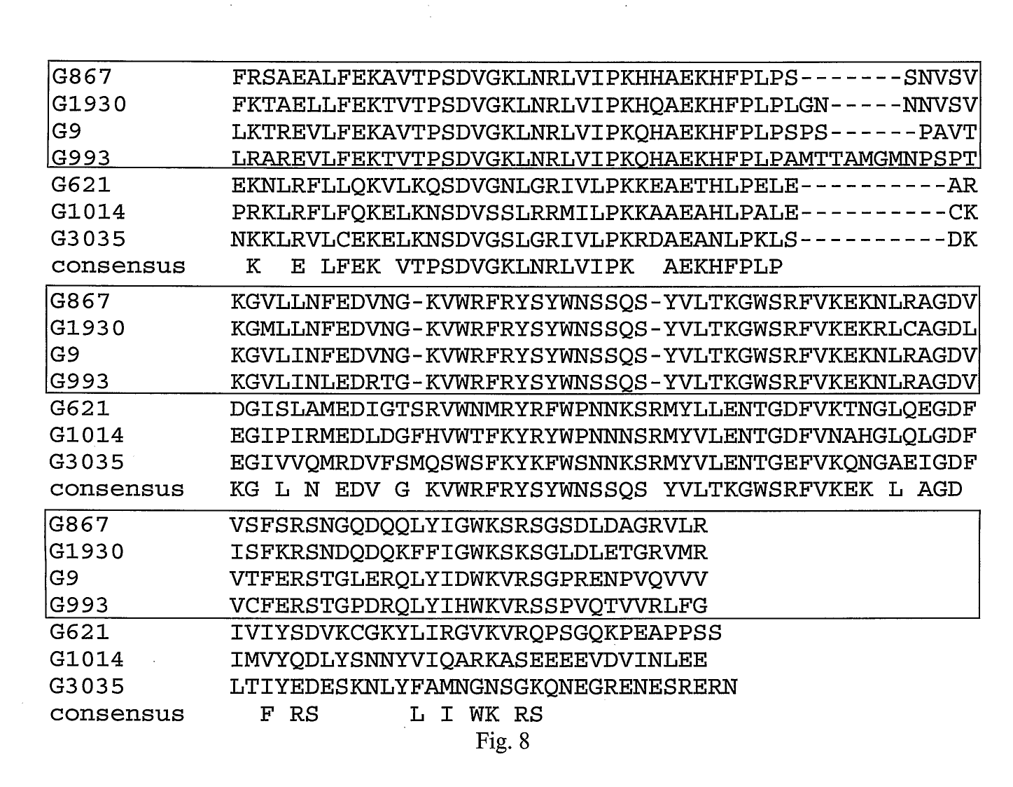

AP2 DNA binding domain. The AP2 domain of G867 is localized in the N-terminal region of the protein (FIGS. 7B-7C). The CAACA element recognized by G867 differs from the GCCGCC motif present in ERF (ethylene response factors, Hao et al. (1998); Hao et al. (2002)) target promoters, and from the CCGAC motif involved in regulation of dehydration responsive genes by the CBF/DREB1 and DREB2 group of transcription factors (Sakuma et al. (2002)). In case of the CBF proteins, regions flanking the AP2 domain are very specific and are not found in other Arabidopsis transcription factors. Furthermore, those regions are highly conserved in CBF proteins across species (Jaglo et al. (2001)). The regions flanking the AP2 domain are also highly conserved in G867 and the paralogs G9, G1930, and G993 (SEQ ID NOs: 88, 106, 92 and 90, respectively; FIGS. 7B-7C).

B3 DNA binding domain. The B3 domain is present in several transcription factor families: RAV, ABI3/VP1, and ARF. It has been shown for all three families that the B3 domain is sufficient for DNA binding (Table 5). However, the binding specificity varies significantly. These differences in target specificity are also reflected at the protein level. Although all B3 domains share certain conserved amino acids, there is significant variation between families. The B3 domain of the RAV proteins G867 (RAV1), G9 (RAV2), G1930, and G993 is highly conserved, and substantially more closely related to the ABI3 than to the ARF family. Despite the fact that the B3 domain can bind DNA autonomously (Kagaya et al. (1999); Suzuki et al. (1997)), in general, B3 domain transcription factors interact with their targets via two DNA binding domains (Table 5). In case of the RAV and ABI3 family, the second domain is located on the same protein. It has been shown for ABI3 (G621) that cooperative binding increases not only the specificity but also the affinity of the interaction (Ezcurra et al. (2000)).

TABLE 5

Binding sites for different B3 domains

| | | | 2nd Domain present in | |
|---|---|---|---|---|
| Family | Binding site | Element | protein | Reference |
| RAV | CACCTG | — | AP2 | Kagaya et al. (1997) |
| ABI3 | CATGCATG | RY/G-box | B2 | Ezcurra et al. (2000) |
| ARF | TGTCTC | AuxRE | other TxF | Ulmasov et al. (1997) |

Other protein features. A potential bipartite nuclear localization signal has been identified in the G867 protein. A protein scan also revealed several potential phosphorylation sites.

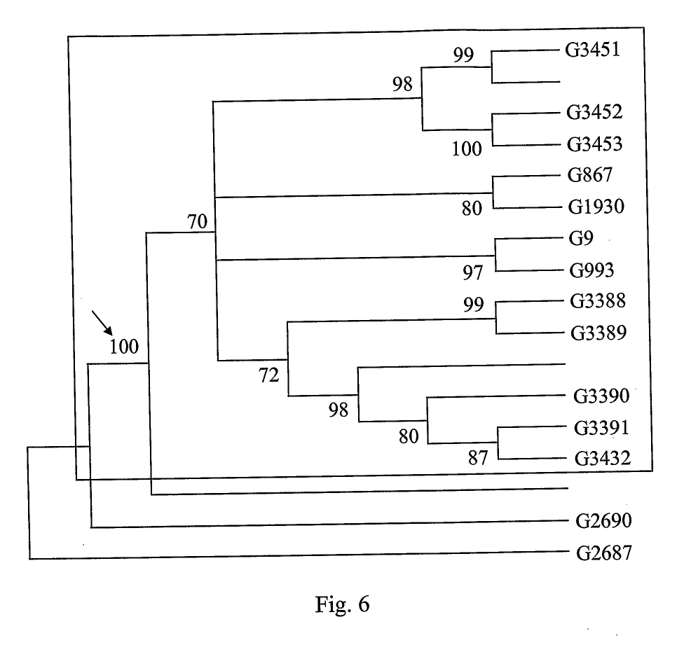

Examination of the alignment of only those sequences in the G867 clade (having monocot and dicot subnodes), indicates 1) a high degree of conservation of the AP2 domains in all members of the clade, 2) a high degree of conservation of the B3 domains in all members of the clade; and 3) a high degree of conservation of an additional motif, the DML motif found between the AP2 and B3 domains in all members of the clade: (H/R S K Xa E/G I/V V D M L R K/R H T Y Xa E/D/N E L/F Xa Q/H S/N/R/G (where Xa is any amino acid), constituting positions 135-152 in G867 (SEQ ID NO: 88). As a conserved motif found in G867 and its paralogs, the DML motif was used to identify additional orthologs of SEQ ID NO: 88. A significant number of sequences were found that had a minimum of 71% identity to the 22 residue DML motif of G867. The DML motif (FIGS. 7C-7D) between the AP2 and B3 DNA binding domain is predicted to have a particularly flexible structure. This could explain the observation that binding of the bipartite motif occurs with similar efficiency, irrespective of the spacing and the orientation of the two motifs (the distance between both elements can vary from 2-8 bp, Kagaya et al. (1999)). Importantly, the DML motif (FIG. 7C-7D) located between the AP2 domain and the B3 domain is not conserved between the G867 clade and the remaining two RAV genes, G2687, SEQ ID NO: 379, and G2690, SEQ ID NO: 381, which form their own separate clade in the phylogenetic analysis (FIG. 6). This motif presumably has a role in determining the unique function of the G867 clade of RAV-like proteins.

Known transcriptional activation domains are either acidic, proline rich or glutamine rich (Liu et al. (1999); the G867 protein does not contain any obvious motifs of these types. Repression domains are relatively poorly characterized in plants, but have been reported for some AP2/ERF (Ohta et al. (2001)) factors. The transcription factors AtERF3 and AtERF4 contain a conserved motif ((L/F)DLN(L/F)xP) which is essential for repression (Ohta et al. (2001)). Such a motif is not found in the G867 protein. Transcriptional repression domains have also been reported for some of the ARF-type B3 domain transcription factors (Tiwari et al. (2001); Tiwari et al. (2003)). Following the N-terminal DNA binding domain, ARFs contain a non-conserved region referred to as the middle region (MR), which has been proposed to function as a either a transcriptional repression or an activation domain, depending on the particular protein. Those ARF proteins with a Q rich MR behave as transcriptional activators, whereas most, if not all other ARFs, function as repressors. However, a well-defined repression motif has yet to be identified. (Tiwari et al. (2001); Tiwari et al. (2003)).

In conclusion, it remains to be resolved whether G867 acts as a transcriptional activator or repressor. It is possible that the protein itself does not contain a regulatory motif, and that its function is a result of either restricting access to certain promoters or the interaction with other regulatory proteins.

### Background Information for G28, the G28 Clade, and Related Sequences

G28 (SEQ ID NO: 147) corresponds to AtERF1 (GenBank accession number AB008103) (Fujimoto et al. (2000)). G28 appears as gene At4 gl7500 in the annotated sequence of Arabidopsis chromosome 4 (AL161546.2). G28 has been shown to confer resistance to both necrotrophic and biotrophic pathogens. G28 (SEQ ID NO: 148) is a member of the B-3a subgroup of the ERF subfamily of AP2 transcription factors, defined as having a single AP2 domain and having specific residues in the DNA binding domain that distinguish this large subfamily (65 members) from the DREB subfamily (see below). AtERF1 is apparently orthologous to the AP2 transcription factor Pti4, identified in tomato, which has been shown by Martin and colleagues to function in the Pto disease resistance pathway, and to confer broad-spectrum disease resistance when overexpressed in Arabidopsis (Zhou et al. (1997); Gu et al. (2000); Gu et al. (2002)).

AP2 domain transcription factors. This large transcription factor gene family includes 145 transcription factors (Weigel (1995); Okamuro et al. (1997); Riechmann and Meyerowitz (1998); Riechmann et al. (2000)). Based on the results of our earlier genomics screens it is clear that this family of proteins affect the regulation of a wide range of morphological and physiological processes, including the acquisition of abiotic and biotic stress tolerance. The AP2 family can be further sub-divided as follows:

[1] The APETALA2 (“C”) class (14 genes) is related to the APETALA2 protein itself (Jofuku et al. (1994)), characterized by the presence of two AP2 DNA binding domains.

[2] The AP2/ERF group (125 genes) which contain a single AP2 domain. This AP2/ERF class can be further categorized into three subgroups:

The DREB (“A”) (dehydration responsive element binding) sub-family which comprises 56 genes. Many of the DREBs are involved in regulation of abiotic stress tolerance pathways (Stockinger et al. (1997); Jaglo-Ottosen et al. (1998); Finkelstein et al. (1998); Sakuma et al. (2002)).

The ERF (ethylene response factor) sub-family (“B”) which includes 65 genes, several of which are involved in regulation of biotic stress tolerance pathways (Ohme-Takagi and Shinshi (1995); Zhou et al. (1997)). The DREB and ERF sub-groups are distinguished by the amino acids present at position 14 and 19 of the AP2 domain: while DREBs are characterized by Val-14 and Glu-19, ERFs typically have Ala-14 and Asp-19. Recent work indicated that those two amino acids have a key function in determining the target specificity (Sakama et al. (2002), Hao et al. (2002)).

[3] The RAV class (6 genes) all of which have a B3 DNA binding domain in addition to the AP2 DNA binding domain, and which also regulate abiotic stress tolerance pathways.

The role of ERF transcription factors in stress responses: ERF transcription factors in disease resistance. The first indication that members of the ERF group might be involved in regulation of plant disease resistance pathways was the identification of Pti4, Pti5 and Pti6 as interactors with the tomato disease resistance protein Pto in yeast 2-hybrid assays (Zhou et al. (1997)). Since that time, many ERF genes have been shown to enhance disease resistance when overexpressed in Arabidopsis or other species. These ERF genes include ERF1 (G1266) of Arabidopsis (Berrocal-Lobo et al. (2002); Berrocal-Lobo and Molina, (2004)); Pti4 (Gu et al. (2002)) and Pti5 (He et al. (2001)) of tomato; Tsi1 (Park et al. (2001); Shin et al. (2002)), NtERF5 (Fischer and Droge-Laser (2004)), and OPBP1 (Guo et al. (2004)) of tobacco; CaERFLP1 (Lee et al. (2004)) and CaPF1 (Yi et al. (2004)) of hot pepper; and AtERF1 (G28) and TDR1 (G1792) of Arabidopsis (our data).

ERF transcription factors in abiotic stress responses. While ERF transcription factors are primarily recognized for their role in biotic stress response, some ERFs have also been characterized as being responsive to abiotic stress. For example, Fujimoto et al. (2000) have shown that AtERF1-5 (corresponding to GIDs: G28 (SEQ ID NO: 148), G1006 (SEQ ID NO: 152), G1005 (SEQ ID NO: 390), G6 (SEQ ID NO: 386) and G1004 (SEQ ID NO: 388) respectively) can respond to various abiotic stresses, including cold, heat, drought, ABA, cycloheximide, and wounding. In addition, several ERF transcription factors that enhance disease resistance when overexpressed also enhance tolerance to various types of hyperosmotic stress. The first published example of this phenomenon was the tobacco gene Tsi1, which was isolated as a salt-inducible gene, and found to enhance salt tolerance and resistance to Pseudomonas syringae pv. tabaci when overexpressed in tobacco (Park et al. (2001)), and resistance to several other pathogens when overexpressed in hot pepper (Shin et al. (2002)). A number of other ERFs have now been shown to confer some degree of disease resistance and hyperosmotic stress tolerance when overexpressed, including OPBP1 of tobacco, which enhances salt tolerance when overexpressed (Guo et al. (2004a)), CaPF1 of hot pepper, which produces freezing tolerance when overexpressed (Yi et al. (2004)), and CaERFLP1 of hot pepper, which enhances salt tolerance when overexpressed (Lee et al. (2004a)). These proteins represent different subclasses of ERFs: Tsi1 is an ERFB-5, OPBP1 is an ERFB-3c, and CaPF1 and CaERFLP1 are in the ERF-B2 class, demonstrating that the capacity to enhance biotic and abiotic stress tolerance is distributed throughout the ERF family.

Regulation of ERF transcription factors by pathogen and small molecule signaling. ERF genes show a variety of stress-regulated expression patterns. Regulation by disease-related stimuli such as ethylene (ET), jasmonic acid (JA), salicylic acid (SA), and infection by virulent or avirulent pathogens has been shown for a number of ERF genes (Fujimoto et al. (2000); Gu et al. (2000); Chen et al. (2002a); Cheong et al., (2002); Onate-Sanchez and Singh (2002); Brown et al. (2003); Lorenzo et al. (2003)). However, some ERF genes are also induced by wounding and abiotic stresses, as discussed above (Fujimoto et al. (2000); Park et al. (2001); Chen et al. (2002a); Tournier et al. (2003)). Currently, it is difficult to assess the overall picture of ERF regulation in relation to phylogeny, since different studies have concentrated on different ERF genes, treatments and time points.

Significantly, several ERF transcription factors that confer enhanced disease resistance when overexpressed, such as ERF1 (G1266), Pti4, and AtERF1 (G28), are transcriptionally regulated by pathogens, ET, and JA (Fujimoto et al. (2000); Onate-Sanchez and Singh (2002); Brown et al. (2003); Lorenzo et al. (2003)). ERF1 is induced synergistically by ET and JA, and induction by either hormone is dependent on an intact signal transduction pathway for both hormones, indicating that ERF1 may be a point of integration for ET and JA signaling (Lorenzo et al. (2003)). At least 4 other ERFs are also induced by JA and ET (Brown et al. (2003)), implying that other ERFs are probably also important in ET/JA signal transduction. A number of the ERF proteins in subgroup 1, including AtERF3 and AtERF4, are thought to act as transcriptional repressors (Fujimoto et al. (2000)), and these two genes were found to be induced by ET, JA, and an incompatible pathogen (Brown et al. (2003)). The net transcriptional effect on these pathways may be balanced between activation and repression of target genes.

The SA signal transduction pathway can act antagonistically to the ET/JA pathway. Interestingly, Pti4 and AtERF1 (G28) are induced by SA as well as by JA and ET (Gu et al. (2000); Onate-Sanchez and Singh (2002)). Pti4, Pti5 and Pti6 have been implicated indirectly in regulation of the SA response, perhaps through interaction with other transcription factors, since overexpression of these genes in Arabidopsis induced SA-regulated genes without SA treatment and enhanced the induction seen after SA treatment (Gu et al. (2002)).

Post-transcriptional regulation of ERF genes by phosphorylation may be a significant form of regulation. Pti4 has been shown to be phosphorylated specifically by the Pto kinase, and this phosphorylation enhances binding to its target sequence (Gu et al. (2000)). Recently, the OsEREBP1 protein of rice has been shown to be phosphorylated by the pathogen-induced MAP kinase BWMK1, and this phosphorylation was shown to enhance its binding to the GCC box (Cheong et al. (2003)), suggesting that phosphorylation of ERF transcription factors may be a common theme. A potential MAPK phosphorylation site has been noted in AtERF5 (Fujimoto et al. (2000)).

Protein structure and properties. G28 lacks introns and encodes a 266 amino acid protein with a predicted molecular weight of 28.9 kDa. Specific conserved motifs have been identified through alignments with other related ERFs (e.g., FIGS. 11A-11B and FIGS. 13D-13E).

AP2 DNA binding domain. The AP2 domain of G28 is relatively centrally positioned in the intact protein (FIGS. 13D-13E). G28 has been shown to bind specifically to the AGCCGCC motif (GCC box: Hao et al. (1998); Hao et al. (2002)). Our analysis of the G28 regulon by global transcript profiling is consistent with this, as the 5′ regions of genes up-regulated by G28 are enriched for the presence of AGCCGCC motifs. The AP2 domain of AtERF1 (G28) was purified and used by Allen et al. (1998) in solution NMR studies of the AP2 domain and its interaction with DNA. This analysis indicated that certain residues in three beta-strands are involved in DNA recognition, and that an alpha helix provides structural support for the DNA binding domain.

Other protein features. A potential bipartite nuclear localization signal has been reported in the G28 protein. A protein scan also revealed several potential phosphorylation sites, but the conserved motifs used for those predictions are small, have a high probability of occurrence. However, the orthologous Pti4 sequence has been shown to be phosphorylated in multiple locations, which have yet to be mapped in detail. A protein alignment of closely related ERF sequences indicates the presence of conserved domains unique to B-3a ERF proteins. For example, a motif not found in other Arabidopsis transcription factors is found directly C-terminal to the AP2 domain in dicot sequences, but is not found in monocot sequences. Another conserved motif is found 40-50 amino acids N-terminal to the AP2 DNA binding domain. The core of this motif is fairly well conserved in both dicots and monocots, but extensions of the motif are divergent between dicots and monocots. The identification of specific motifs unique to small clades of ERF transcription factors suggests that these motifs may be involved in specific interactions with other protein factors involved in transcriptional control, and thereby may determine functional specificity. Known transcriptional activation domains are either acidic, proline rich or glutamine rich (Liu et al. (1999)). The G28 protein contains one acid-enriched region (overlapping with the first dicot-specific motif). There is also evidence that regions rich in serine, threonine, and proline may function in transcriptional activation (Silver et al. (2003)). There are two ser/pro-enriched regions in the region N-terminal to the AP2 domain. None of these domains has yet to be demonstrated directly to have a role in transcriptional activation.

Our Earlier Discoveries related to G28. G28 is included in the current disease program based on the enhanced tolerance of 35S::G28 lines to Sclerotinia, Botrytis, and Erysiphe demonstrated in our earlier genomics program. Resistance to Sclerotinia, and Botrytis was confirmed in the present soil-based assays. Follow-up work also demonstrated enhanced tolerance to Phytophthora capsisci (data not shown).

Further testing confirmed that this increased disease resistance is not achieved at the expense of susceptibility to other pathogens (e.g., Pseudomonas syringae and Fusarium oxysporum). Although no significant growth penalty was observed with the initial transgenic lines studied in the genomics program, subsequent analysis of a larger population of transgenic lines in the phase I SBIR program revealed a detectable growth penalty, particularly during early growth stages. The magnitude of this growth penalty correlated with expression level as measured by quantitative RT-PCR. A slight delay in flowering (1 to 2 days) was also observed at the highest expression levels. We observed no differences between G28 overexpressing plants and wild-type plants in germination efficiency, number of leaves per plant, inflorescence weight, silique weight, or chlorophyll content.

Regulation of G28. Induction of G28 (AtERF1) by pathogens, ethylene, methyl jasmonate, and salicylic acid has been published (Chen et al. (2002a); Fujimoto et al. (2000); Onate-Sanchez and Singh (2002)). Our RT-PCR experiments have confirmed induction by Botrytis, SA and JA (data not shown).

### Background Information for G1792, the G1792 Clade, and Related Sequences

G1792 (SEQ ID NO: 221, 222) is part of both the drought and disease programs. Background information relevant to each of these traits is presented below.

We first identified G1792 (AT3G23230) as a transcription factor in the sequence of BAC clone K14B15 (AB025608, gene K14B15.14). We have assigned the name TRANSCRIPTIONAL REGULATOR OF DEFENSE RESPONSE 1 (TDR1) to this gene, based on its apparent role in disease responses. The G1792 protein contains a single AP2 domain and belongs to the ERF class of AP2 proteins. A review of the different sub-families of proteins within the AP2 family is provided in the information provided for G28, above. The G28 disclosure provided herein includes description of target genes regulated by ERF transcription factors, the role of ERF transcription factors in stress responses: ERF transcription factors in disease resistance, ERF transcription factors in abiotic stress responses, regulation of ERF transcription factors by pathogen and small molecule signaling, etc., which also pertain to G1792.

G1792 overexpression increases survivability in a soil-based drought assay. 35S::G1792 lines exhibited markedly enhanced drought tolerance in a soil-based drought screen compared to wild-type, both in terms of their appearance at the end of the drought period, and in survival following re-watering.

G1792 overexpression produces disease resistance. 35S::G1792 plants were more resistant to the fungal pathogens Fusarium oxysporum and Botrytis cinerea: they showed fewer symptoms after inoculation with a low dose of each pathogen. This result was confirmed using individual T2 lines. The effect of G1792 overexpression in increasing resistance to pathogens received further, incidental confirmation. T2 plants of 35S::G1792 lines 5 and 12 were being grown (for other purposes) in a room that suffered a serious powdery mildew infection. For each line, a pot of 6 plants was present in a flat containing 9 other pots of lines from unrelated genes. In either of the two different flats, the only plants that were free from infection were those from the 35S::G1792 line. This observation suggested that G1792 overexpression increased resistance to powdery mildew.

G1792 overexpression increases tolerance to growth on nitrogen-limiting conditions. 35S::G1792 transformants showed more tolerance to growth under nitrogen-limiting conditions. In a root growth assay under conditions of limiting N, 35S::G1792 lines were slightly less stunted. In an germination assay that monitors the effect of carbon on nitrogen signaling through anthocyanin production (with high sucrose+/−glutamine; Hsieh et al. (1998)), the 35S::G1792 lines made less anthocyanin on high sucrose (+glutamine), suggesting that the gene could be involved in the plants ability to monitor carbon and nitrogen status.

G1792 overexpression causes morphological alterations. Plants overexpressing G1792 showed several mild morphological alterations: leaves were dark green and shiny, and plants bolted, and subsequently senesced, slightly later than wild-type controls. Among the T1 plants, additional morphological variation (not reproduced later in the T2 plants) was observed: many showed reductions in size as well as aberrations in leaf shape, phyllotaxy, and flower development.

Follow-up work in disease. G1792 has three potential paralogs, G30, G1791 and G1795 (SEQ ID NOs: 226, 230, and 224, respectively), which were not assayed for disease resistance in the genomics program because their overexpression caused severe negative side effects. Some evidence suggested that these genes might play a role in disease resistance: expression of G1795 and G1791 was induced by Fusarium, and G1795 by salicylic acid, in RT-PCR experiments, and the lines shared the glossy phenotype observed for G1792. Phylogenetic trees based on whole protein sequences do not always make the relationship of these proteins to G1792 clear; however, the close relationship of these proteins is evident in an alignment (FIG. 11A-11B, FIG. 19) and in a phylogenetic analysis (FIG. 18) based on the conserved AP2 domain and a second conserved motif (FIG. 19; the EDLL domain described below).

G1792, G1791, G1795 and G30 were expressed under the control of four different promoters using the two-component system. The promoters chosen were 35S, RBCS3 (mesophyll or photosynthetic-specific), LTP1 (epidermal-specific), and 35S::LexA:GAL4:GR (dexamethasone-inducible). All promoters other than 35S produced substantial amelioration of the negative side effects of transcription factor overexpression.

Five lines for each combination were tested with Sclerotinia, Botrytis, or Fusarium. Interestingly, G1791 and G30 conferred significant resistance to Sclerotinia when expressed under RBCS3 or 35S::LexA:GAL4:GR, even though G1792 does not confer Sclerotinia resistance. These results support the hypothesis that genes of this clade confer disease resistance when expressed under tissue specific or inducible promoters.

TABLE 6
Disease screening of G1792 and paralogs under different promoters

| | G1792 | | | G1791 | | | G1795 | | | G30 | | |
|---|---|---|---|---|---|---|---|---|---|---|---|---|
| | SEQ ID NO: | | | | | | | | | | | |
| | 222 | | | 230 | | | 224 | | | 226 | | |
| | B | S | F | B | S | F | B | S | F | B | S | F |
| 35S | ++ | wt | + | nd | nd | nd | nd | nd | nd | nd | nd | nd |
| RBCS3 | + | wt | + | wt | wt | wt | ++ | ++ | wt | + | + | wt |
| LTP1 | wt | wt | nd | + | wt | wt | ++ | + | wt | + | wt | wt |
| Dex-ind. | ++ | wt | + | ++ | ++ | wt | ++ | ++ | wt | ++ | ++ | wt |

Abbreviations and symbols:
B, Botrytis
S, Sclerotinia
F, Fusarium
Scoring:
wt, wild-type (susceptible) phenotype
+, mild to moderate resistance
++, strong resistance
nd, not determined

Domains. In addition to the AP2 domain (domains of G1792 clade members are shown in Table 15), G1792 contains a putative activation domain. This domain (Table 15) has been designated the “EDLL domain” based on four amino acids that are highly conserved across paralogs and orthologs of G1792 (FIG. 19).

Tertiary Structure. The solution structure of an ERF type transcription factor domain in complex with the GCC box has been determined (Allen et. al., 1998). It consists of a β-sheet composed of three strands and an α-helix. Flanking sequences of the AP2 domain of this protein were replaced with the flanking sequences of the related CBF1 protein, and the chimeric protein was found to contain the same arrangement of secondary structural elements as the native ERF type protein (Allen et al. (1998)). This implies that the secondary structural motifs may be conserved for similar ERF type transcription factors within the family.

DNA Binding Motifs. Two amino acid residues in the AP2 domain, Ala-14 and Asp-19, are definitive of the ERF class transcription factors Sakuma et al. (2002). Recent work indicates that these two amino acids have a key function in determining binding specificity (Sakuma et al. (2002), Hao et al. (2002)) and interact directly with DNA. The 3-dimensional structure of the GCC box complex indicates the interaction of the second strand of the β-sheet with the DNA.

### Background Information for G47, the G47 Clade, and Related Sequences

G47 (SEQ ID NO: 173, AT1G22810) encodes a member of the AP2 class of transcription factors (SEQ ID NO: 174) and was included based on the resistance to drought-related abiotic stress exhibited by 35S::G47 Arabidopsis lines and by overexpression lines for the closely related paralog, G2133 (SEQ ID NO: 176, AT1G71520). A detailed genetic characterization has not been reported for either of these genes in the public literature.

AP2 Family transcription factors. Based on the results of our earlier genomics screens, it is clear that this family of proteins affect the regulation of a wide range of morphological and physiological processes, including the acquisition of stress tolerance. The AP2 family can be further divided into subfamilies as detailed in the G28 section, above.

G47 and G2133 protein structure. G47 and G2133 comprise a pair of highly related proteins (FIG. 15) and are members of the AP2/ERF subfamily. Both proteins possess an AP2 domain at the amino terminus and a somewhat acidic region at the C-terminus that might constitute an activation domain. A putative bipartite NLS is located at the start of the AP2 domain in both proteins. Sakuma et al. (Sakuma et al. (2002)) categorized these factors within the A-5 class of the DREB related sub-group based on the presence of a V residue at position 14 within the AP2 domain. Importantly, however, position 19 within the AP2 domain is occupied by a V residue in both G2133 and G47, rather than an E residue, as is the case in the majority of DREBs. Additionally, the “RAYD-box” within the AP2 domains of these two proteins is uniquely occupied by the sequence VAHD (FIG. 15), a combination not found in any other Arabidopsis AP2/ERF protein (Sakuma et al. (2002)). These differences to other AP2 proteins could confer unique DNA binding properties on G2133 and G47.

Discoveries made in earlier genomics programs. We initially identified G47 in 1998, as an AP2 domain protein encoded within the sequence of BAC T22J18 (GenBank accession AC003979) released by the Arabidopsis Genome Initiative. We then confirmed the boundaries of the gene by RACE and cloned a full-length cDNA clone by RT-PCR. G2133 was later identified within BAC F3I17 (GenBank accession AC016162) based on its high degree of similarity to G47. Both genes were analyzed by overexpression analysis during our earlier genomics program.

Morphological effects of G47 and G2133 overexpression. A number of striking morphological effects were observed in 35S::G47 lines. At early stages, the plants were somewhat reduced in size. However, these lines flowered late and eventually developed an apparent increase in rosette size compared to mature wild-type plants. Additionally, the 35S::G47 plants showed a marked difference in aerial architecture; inflorescences displayed a short stature, had a reduction in apical dominance, and developed thick fleshy stems. When sections from these stems were stained and examined, it was apparent that the vascular bundles were grossly enlarged compared to wild-type. Similar morphological changes were apparent in shoots of 35S::G2133 lines, but most of the 35S::G2133 lines exhibited much more severe dwarfing at early stages compared to 35S::G47 lines. Nevertheless, at later stages, a number of 35S::G2133 lines showed a very similar reduction of apical dominance and a fleshy appearance comparable to that seen in 35S::G47 lines.

Physiological effects of G47 and G2133 overexpression. Both 35S::G2133 lines and 35S::G47 lines exhibited abiotic stress resistance phenotypes in the screens performed during our earlier genomics program. 35S::G47 lines displayed increased tolerance to hyperosmotic stress (PEG) whereas 35S::G2133 lines were more tolerant to the herbicide glyphosate compared to wild type.

The increased tolerance of 35S::G47 lines to PEG, combined with the fleshy appearance and altered vascular structure of the plants, led us to test these lines in a soil drought screen. 35S::G2133 lines were also included in that assay, given the close similarity between the two proteins and the comparable morphological effects obtained. Both 35S::G47 and 35S::G2133 lines showed a strong performance in that screen and exhibited markedly enhanced drought tolerance compared to wild-type, both in terms of their appearance at the end of the drought period, and in survivability following re-watering. In fact, of the approximately 40 transcription factors tested in that screen, 35S::G2133 lines showed the top performance in terms of each of these criteria.

### Background Information for G1274, the G1274 Clade, and Related Sequences

G1274 (SEQ ID NO: 185) from Arabidopsis encodes a member of the WRKY family of transcription factors (SEQ ID NO: 186) and was included based primarily on soil-based drought tolerance exhibited by 35S::G1274 Arabidopsis lines. G1274 corresponds to AtWRKY51 (At5g64810), a gene for which there is currently no published information.

WRKY transcription factors. WRKY genes appear to have originated in primitive eukaryotes such as Giardia lamblia, Dictyostelium discoideum, and the green alga Chliamydomonas reinhardtii, and have since greatly expanded in higher plants (Zhang and Wang (2005)). In Arabidopsis alone, there are more than 70 members of the WRKY superfamily. The defining feature of the family is the ˜57 amino acid DNA binding domain that contains a conserved WRKYGQK heptapeptide motif. Additionally, all WRKY proteins have a novel zinc-finger motif contained within the DNA binding domain. There are three distinct groups within the superfamily, each principally defined by the number of WRKY domains and the structure of the zinc-finger domain (reviewed by Eulgem et al. (2000)). Group I members have two WRKY domains, while Group II members contain only one. Members of the Group II family can be further split into five distinct subgroups (IIa-e) based on conserved structural motifs. Group III members have only one WRKY domain, but contain a zinc finger domain that is distinct from Group II members. The majority of WRKY proteins are Group II members, including G1274 and the related genes being studied here. An additional common feature found among WRKY genes is the existence of a conserved intron found within the region encoding the C-terminal WRKY domain of group I members or the single WRKY domain of group II/III members. In G1274, this intron occurs between the sequence encoding amino acids R130 and N131.

The founding members of the WRKY family are SPF1 from sweet potato (Ishiguro and Nakamura, 1994), ABF1/2 from oat (Rushton et al. (1995)), PcWRKY1,2,3 from parsley (Rushton et al. (1996)) and ZAP1 from Arabidopsis (de Pater et al. (1996)). These proteins were identified based on their ability to bind the so-called W-box promoter element, a motif with the sequence (T)(T)TGAC(C/T). Binding of WRKY proteins to this motif has been demonstrated both in vivo and in vitro (Rushton et al. (1995); de Pater et al. (1996); Eulgem et al., (1999); Yang et al. (1999); Wang et al. (1998). Additionally, the solution structure of the WRKY4 protein (G884, AT1G13960) has recently been reported (Yamasaki et al. (2005)). In this study, a DNA titration experiment strongly indicates that the conserved WRKYGQK sequence is directly involved in DNA binding. This element is remarkably conserved, and found in many genes associated with the plant defense response.

The two WRKY domains of Group I members appear functionally distinct, and it is the C-terminal sequence that appears to mediate sequence-specific DNA binding. The function of the N-terminal domain is unclear, but may contribute to the binding process, or provide an interface for protein-protein interactions. The single WRKY domain in Group II members appears more like the C-terminal domain of Group I members, and likely performs the similar function of DNA binding.

Structural features of G1274. The primary amino acid sequences for the predicted G1274 protein and related polypeptides are presented in FIG. 17A-17H. The G1274 sequence possesses a potential serine-threonine-rich activation domain and putative nuclear localization signals. The “WRKY” (DNA binding) domain, indicated by the horizontal line and the angled arrow “ [symbol] ”, and zinc finger motif, with the pattern of potential zinc ligands C-X₄₋₅-C—X₂₂₋₂₃-H-X₁-H, indicated by boxes in FIGS. 17E-17F, are also shown.

Discoveries made in earlier genomics programs. G1274 expression in wild-type plants was detected in leaf, root and flower tissue. Expression of G1274 was also enhanced slightly by hyperosmotic and cold stress treatments, and by auxin or ABA application. Additionally, the gene appears induced by Erysiphe infection and salicylic acid treatment, consistent with the known role of WRKY family members in defense responses. The closely related gene G1275 (SEQ ID NO: 207) is strongly repressed in wild-type plants during soil drought, and remains significantly down-regulated compared to well-watered plants even after rewatering.

In G1274 overexpression studies, transformed lines were more tolerant to low nitrogen conditions and were less sensitive to chilling than wild-type plants. G1274 overexpressing seedlings were also hits in a C:N sensing screen, indicating that G1274 may alter the plants ability to modulate carbon and/or nitrogen uptake and utilization. G1274 overexpression also produced alterations in inflorescence and leaf morphology. Approximately 20% of overexpressors were slightly small and developed short inflorescences that had reduced internode elongation. Overall, these plants were bushier and more compact in stature than wild-type plants. In T2 populations, rosettes of some 35S::G1274 plants were distinctly broad with greater biomass than wild-type.

35S::G1274 plants also out-performed wild-type plants in a soil drought assay; these results are presented in greater detail in Example XIII.

Overexpression of G1275 (AtWRKY50), a gene closely related to G1274 and also being studied here, had a more severe effect on morphology than G1274. 35S::G1275 plants were small, with reduced apical dominance and stunted inflorescences. While the plants were fertile, seed yield was low and these plants were not tested in physiological assays. In wild-type plants, this gene, similar to G1274, appeared to be induced by various stresses, but had a different overall expression pattern. G1275 was primarily expressed in rosettes and siliques, and had lower but detectable expression in shoots, roots, flowers and embryos.

The final Arabidopsis gene included in this study group, G1758 (SEQ ID NO: 393, AtWRKY59) was highly induced by salicylic acid, and slightly by Erysiphe and auxin, but no other treatments or stresses. In wild-type plants, this gene is primarily expressed in roots, rosettes, siliques and germinating seedlings. Morphologically and physiologically, 35S::G1758 plants were similar to wild-type.

In general, there have been several studies that indicate WRKY genes are induced by a wide variety of abiotic stresses (Zhang and Wang (2005)), including drought (Pnueli et al. (2002); Mare et al. (2004); Zou et al. (2004)). However, to date, there are no examples in the literature of cases where altered expression of WRKY proteins has been directly used to provide drought tolerance.

### Background Information for G2999, the G2999 Clade, and Related Sequences

G2999 (SEQ ID NO: 255, AT2G18350) encodes a member of the ZF-HD class of transcription factors ((SEQ ID NO: 256) and was included based on the resistance to drought-related abiotic stress exhibited by 35S::G2999 lines.

Identification of ZF-HD transcription factors and their role in plants. The ZF-HD family of transcriptional regulators was identified by Windhovel et al. (2001), while studying the regulatory mechanisms responsible for the mesophyll-specific expression of the C4 phosphoenolpyruvate carboxylase (PEPC) gene from the genus Flavaria. Using a yeast one-hybrid screen, these workers recovered five cDNA clones, which encoded proteins capable of activating the promoter of the Flavaria C4 PEPC gene. One of the five clones encoded histone H4. However, the remaining four clones (FtHB1 [GenBank accession=Y18577, our “GID” identifier=G3859, SEQ ID NO: 413], FbHB2 [GenBank accession=Y18579, our “GID” identifier=G3668, SEQ ID NO: 415], FbHB3 [GenBank accession=Y18580, our “GID” identifier=G3860, SEQ ID NO: 417], and FbHB4 [GenBank accession ═Y18581, our “GID” identifier=G3861, 419]) all encoded a novel type of protein that contained two types of highly conserved domains. At the C-termini, a region was apparent that had many of the features of a homeodomain, whereas at the N-termini, two zinc finger motifs were present. Given the presence of zinc fingers and the potential homeodomain, Windhovel et al. (2001), named the new family of proteins as the ZF-HD group.

Using BLAST searches we have identified a variety of ZF-HD proteins from a variety of other species, including rice and corn (FIG. 20 and FIGS. 21A-21J).

Structural features of ZF-HD proteins. The primary amino acid sequence of the G2999 product, showing the relative positions of the ZF and HD domains, is presented in FIGS. 21D-21E and FIGS. 21H-21I. G2999 comprises an acidic region at the N-terminus which might represent an activation domain and a number of motifs which might act as nuclear localization signals.

Secondary structure analyses performed by Windhovel et al. (Windhovel et al. (2001)) revealed that the putative homeodomains of the newly identified ZF-HD proteins contained three alpha helices with features similar to those in the classes of homeodomain already known in plants (Duboule (1994); Burglin (1997); Burglin (1998)). Interestingly, though, if full-length proteins of the ZF-HD group are BLASTed against plant protein databases, they do not preferentially align with known classes of plant homeodomain proteins. In fact, the ZF-HD proteins from plants appear to be more closely related to the LIM homeodomain proteins from animals than any of the previously known classes of plant homeodomain proteins (Windhovel et al. (2001)).

It is well established that homeodomain proteins are transcription factors, and that the homeodomain is responsible for sequence specific recognition and binding of DNA (Affolter et al. (1990); Hayashi and Scott (1990), and references therein). Genetic and structural analysis indicate that the homeodomain operates by fitting the most conserved of three alpha helices, helix 3, directly into the major groove of the DNA (Hanes and Brent (1989); Hanes and Brent (1991); Kissinger et al. (1990); Wolberger et al. (1991); Duboule (1994)). A large number of homeodomain proteins have been identified in a range of higher plants (Burglin (1997); Burglin (1998)), and we will define these as containing the ‘classical’ type of homeodomain. These all contain the signature WFXNX[RX] (X=any amino acid, [RK] indicates either an R or K residue at this position) within the third helix.

Data from the Genome Initiative indicate that there are around 90 “classical” homeobox genes in Arabidopsis. These are now being implicated in the control of a host of different processes. In many cases, plant homeodomains are found in proteins in combination with additional regulatory motifs such as leucine zippers. Classical plant homeodomain proteins can be broadly categorized into the following different classes based on homologies within the family, and the presence of other types of domain: KNOX class I, KNOX class II, HD-BEL1, HD-ZIP class I, HD-ZIP class II, HD-ZIP class III, HD-ZIP class IV (GL2 like), PHD finger type, and WUSCHEL-like (Freeling and Hake (1985); Vollbrecht et al. (1991); Schindler et al. (1993); Sessa et al. (1994); Kerstetter et al. (1994); Kerstetter et al. (1997); Burglin (1997); Burglin (1998); Schoof et al. (2000)). A careful examination of the ZF-HD proteins reveals a number of striking differences to other plant homeodomains. The ZF-HD proteins all lack the conserved F residue within the conserved WFXNX[RK] (X=any amino acid, [RK] indicates either an R or K residue at this position) motif of the third helix. Additionally, there are four amino acids inserted in the loop between first and second helices of the ZF-HD proteins, whereas in other HD proteins there are a maximum of three amino acids inserted in this position (Burglin (1997)). When these homeodomains are aligned with classical homeodomains from plants, they form a very distinct clade within the phylogeny (FIGS. 20 and 21H-21I). Thus, these structural distinctions within the homeodomain could confer functional properties on ZF-HD proteins that are different to those found in other HD proteins.

The zinc finger motif at the N-terminus is highly conserved across the ZF-HD family. An alignment showing this region from the 14 Arabidopsis ZF-HD proteins and selected ZF-HD proteins from other species is shown in FIGS. 21D-21E and 21H-21I. Yeast two-hybrid experiments performed by Windhovel et al. (2001) demonstrated that ZF-HD proteins form homo and heterodimers through conserved cysteine residues within this region.

Homeodomain transcription factors that also possess a zinc finger domain exist in animals (Mackay and Crossley (1998)) and these include the LIM homeodomains. In fact the plant ZF-HD factors are more closely related to the animal LIM homeodomains than they are to the other classes of plant homeodomain proteins (Windhovel et al. (2001)). However, the ZF regions of the animal proteins are very different to those in the plant ZF-HD factors, and substantial similarity is only found within the homeodomain.

Discoveries made in earlier genomics programs. Following the publication of the Windhovel et al. (2001) study, we identified fourteen ZF-HD factors in the Arabidopsis genome sequence. An alignment of the full-length proteins and a phylogenetic tree based on that alignment are shown in FIGS. 21A-21J. Analysis of ZF-HB genes was performed. None of the genes were analyzed by KO analysis, but we examined the phenotypes of Arabidopsis overexpression lines for 12 of the 14 family members. Compared to other transcription factor families, the ZF-HD family yielded a disproportionate number of abiotic stress related phenotypes, with 6 of the 12 genes analyzed, generating phenotypes in this category (Table 7).

TABLE 7
Summary of results of overexpression of the Arabidopsis
ZF-HD family members obtained during genomics screens

| | SEQ ID | Morphological phenotypes obtained on | Abiotic stress related phenotypes obtained on |
|---|---|---|---|
| GID | NO: | overexpression during genomics screens | overexpression during genomics screens |
| G2989 | 280 | Early flowering noted, but phenotype | Wild-type |
| | | variable between lines and generations | |
| G2990 | 284 | Wild-type | Altered response to growth on low N media |
| G2991 | 282 | Some dwarfing and retarded growth, but | Wild-type |
| | | phenotype variable between lines and | |
| | | generations | |
| G2992 | 286 | Early flowering and reduced size | Increased NaCl tolerance in germination |
| | | | assay; increased anthocyanin production in |
| | | | C/N sensing assay; slight chlorosis when |
| | | | grown on MS media |
| G2993 | 276 | Reduced size, slow development, | Decreased hyperosmotic stress tolerance in |
| | | delayed flowering, dark coloration | germination assay; increased sensitivity to |
| | | | growth in cold; reduced secondary root |
| | | | growth on MS media |
| G2994 | | Wild-type | Wild-type |
| G2995 | 288 | Not analyzed | Not analyzed |
| G2996 | 270 | Some size variation between lines | Decreased tolerance to growth on mannitol |
| | | | media |
| G2997 | 264 | Some size variation between lines | Wild-type |
| G2998 | 258 | Delayed flowering | Increased NaCl tolerance in germination assay |
| G2999 | 256 | Wild-type | Increased NaCl tolerance in growth assay |
| G3000 | 260 | Not analyzed | Not analyzed |
| G3001 | 272 | Wild-type | Wild-type |
| G3002 | 290 | Early flowering noted, but phenotype | Wild-type |
| | | variable between lines and generations | |

G2999 was initially included as a candidate for the drought program based on the enhanced salt tolerance observed in overexpression lines for G2999, and overexpression lines for the closest paralog, G2998. Overexpression lines for a third gene that is a potential paralog, G3000, were not analyzed during our earlier genomics program. 35S::G2999 lines were subsequently tested in a soil drought assay and showed a good performance in terms of both tolerance to drought and survivability following re-watering at the end of a drought period (Example XIII). Lines for the ZF-HD family members G2992 and G2998 were also included in the soil drought screen. Lines for both of these genes showed improved drought resistance compared to wild-type (in terms of their appearance at the end of a drought treatment), but showed a somewhat lower survivability to the drought than controls following re-watering.

### Background Information for G3086, the G3086 Clade, and Related Sequences

G3086 (SEQ ID NO: 291-292, AT1G51140) confers tolerance to drought related stress as exhibited by 35S::G3086 Arabidopsis lines. No detailed characterization of G3086 has been presented in the public literature.

G3086 belongs to the basic/helix-loop-helix (bHLH) family of transcription factors. This family is defined by the bHLH signature domain, which consists of 60 amino acids with two functionally distinct regions. The basic region, located at the N-terminal end of the domain, is involved in DNA binding and consists of 15 amino acids with a high number of basic residues. The HLH region, at the C-terminal end, functions as a dimerization domain (Murre et al. (1989); Ferre-D'Amare et al. (1994)) and is constituted mainly of hydrophobic residues that form two amphipathic helices separated by a loop region of variable sequence and length (Nair and Burley (2000)). Outside of the conserved bHLH domain, these proteins exhibit considerable sequence divergence (Atchley et al. (1999)). Cocrystal structural analysis has shown that the interaction between the HLH regions of two separate polypeptides leads to the formation of homodimers and/or heterodimers and that the basic region of each partner binds to half of the DNA recognition sequence (Ma et al. (1994); Shimizu et al. (1997)). Some bHLH proteins form homodimers or restrict their heterodimerization activity to closely related members of the family. On the other hand, some can form heterodimers with one or several different partners (Littlewood and Evan (1998).

The core DNA sequence motif recognized by the bHLH proteins is a consensus hexanucleotide sequence known as the E-box (5′-CANNTG-3′). There are different types of E-boxes, depending on the identity of the two central bases. One of the most common is the palindromic G-box (5′-CACGTG-3′). Certain conserved amino acids within the basic region of the protein provide recognition of the core consensus site, whereas other residues in the domain dictate specificity for a given type of E-box (Robinson et al. (2000)). In addition, flanking nucleotides outside of the hexanucleotide core have been shown to play a role in binding specificity (Littlewood and Evan (1998); Atchley et al. (1999); Massari and Murre (2000)), and there is evidence that a loop residue in the protein plays a role in DNA binding through elements that lie outside of the core recognition sequence (Nair and Burley (2000)).

We have identified 153 Arabidopsis genes encoding bHLH transcription factors; together they comprise one of the largest transcription factor gene families. Although several other sequenced eukaryotes also have large bHLH families, when expressed as a percentage of the total genes present in the genome, Arabidopsis has the largest relative representation at 0.56% of the identified genes, compared with yeast (0.08%), Caenorhabditis elegans (0.20%), Drosophila (0.40%), puffer fish (Takifugu rubripes) (0.40%), human (0.40%), and mouse (0.50%). This observation suggests that the bHLH factors have evolved to assume a major role in plant transcriptional regulation. On the other hand, plant bHLHs appear to have evolved a narrower spectrum of variant sequences within the bHLH domain than those of the mammalian systems and appear to lack some of the various ancillary signature motifs, such as the PAS and WRPW domains, found in certain bHLH protein subclasses in other organisms (Riechmann et al. (2000); Ledent and Vervoort (2001); Mewes et al. (2002); Waterston et al. (2002)).

In spite of this large number of genes in the bHLH transcription factor family, relatively few plant bHLH proteins have been described in the public literature to date, and the family remains largely uncharacterized in terms of the identification of its members and the biological processes they control within publicly available data. A genomics based analysis of plant bHLH proteins have recently been the subject of several extensive reviews (Buck and Atchley (2003); Heim et al. (2003); Toledo-Ortiz et al. (2003); Bailey et al. (2003)).

Protein structure. There are two important functional activities determined by the amino acid sequence of the bHLH domain: DNA binding and dimerization. The basic region in the bHLH domain determines the DNA binding activity of the protein (Massari and Murre (2000)). The DNA binding bHLH category can be subdivided further into two subcategories based on the predicted DNA binding sequence: (1) the E-box binders and (2) the non-E-box binders (Toledo-Ortiz et al. (2003)) based on the presence or absence of two specific residues in the basic region: Glu-319 and Arg-321. These residues constitute the E-box recognition motif, because they are conserved in the proteins known to have E-box binding capacity (Fisher and Goding (1992); Littlewood and Evan (1998)). The analysis of the crystal structures of USF, E47, Max, MyoD, and Pho4 (Ellenberger et al. (1994); Ferre-D'Amare et al. (1994); Ma et al. (1994); Shimizu et al. (1997); Fuji et al. (2000)) have shown that Glu-319 is critical because it contacts the first CA in the E-box DNA binding motif (CANNTG). Site-directed mutagenesis experiments with Pho4, in which other residues (Gln, Asp, and Leu) were substituted for Glu-13, demonstrated that the substitution abolished DNA binding (Fisher and Goding (1992)). Meanwhile, the role of Arg-16 is to fix and stabilize the position of the critical Glu-13; therefore, it plays an indirect role in DNA binding (Ellenberger et al. (1994); Shimizu et al. (1997); Fuji et al. (2000)).

The E-box binding bHLHs can be categorized further into subgroups based on the type of E-box recognized. Crystal structures show that the type of E-box binding preferences are established by residues in the basic region, with the best understood case being that of the G-box binders (Ellenberger et al. (1994); Ferre-D'Amare et al. (1994); Shimizu et al. (1997)). Toledo-Ortiz et al. (2003) have subdivided the Arabidopsis E-box binding bHLHs into (1) those predicted to bind G-boxes and (2) those predicted to recognize other types of E-boxes (non-G-box binders). There are three residues in the basic region of the bHLH proteins: His/Lys, Glu, and Arg at positions 315, 319, and 322 which constitute the classic G-box (CACGTG) recognition motif. Glu-319 is the key Glu involved in DNA binding, and analysis of the crystal structures of Max, Pho4, and USF indicates that Arg-322 confers specificity for CACGTG versus CAGCTG E-boxes by directly contacting the central G of the G-box. His-315 has an asymmetrical contact and also interacts with the G residue complementary to the first C in the G-box (Ferre-D'Amare et al. (1994); Shimizu et al. (1997); Fuji et al. (2000)).

Based on this analysis, G3086 is predicted to be an E-box binding protein. However, since it lacks a histidine or lysine at position 315, it is not predicted to be a G-box binding protein.

bHLH proteins are well known to dimerize, but the critical molecular determinants involved are not well defined (Shirakata et al. (1993); Littlewood and Evan (1998); Ciarapica et al. (2003)). On the other hand, the leucine residue at the position equivalent to residue 333 in G3086 has been shown to be structurally necessary for dimer formation in the mammalian Max protein (Brownlie et al. (1997)). This leucine is the only invariant residue in all bHLH proteins, consistent with a similar essential function in plant bHLH protein dimerization (arrow in FIG. 23G). Current information indicates that dimerization specificity is affected by multiple parameters, including hydrophobic interfaces, interactions between charged amino acids in the HLH region, and partner availability, but no complete explanation for partner recognition specificity has been documented (Ciarapica et al. (2003)). Thus, although empirically it seems logical that bHLH proteins most closely related in sequence in the HLH region are the most likely to form heterodimers, there has been no systematic investigation of this possibility to date.

In other eukaryotes, apart from the bHLH domain, additional functional domains have been identified in the bHLH proteins. These additional domains play roles in protein-protein interactions (e.g., PAS, WRPW, and COE in groups C, E, and F, respectively; Dang et al. (1992); Atchley and Fitch (1997); Ledent and Vervoort (2001)) and in bHLH dimerization specificity (e.g., the zipper domain, part of group B). G3086 does not appear to contain any of these functional domains apart from two nuclear localization signal (NLS) motifs. One NLS motif appears to be a simple localization signal, while the other has a bipartite structure, based on the occurrence of lysine and arginine clusters.

An alignment of the full-length proteins for genes in the G3086 study group compared with a selection of other proteins from the HLH/MYC family, and a phylogenetic tree based on that alignment is shown in FIG. 22.

Abiotic stress related phenotypes. G3086 was initially included as a candidate for the drought program based on the enhanced tolerance to salt and heat exhibited by overexpression lines. 35S::G3086 lines were subsequently tested in a soil drought assay. Lines for this gene showed improved drought resistance compared to wild-type in terms of both their appearance at the end of a drought treatment and survivability to drought treatment compared to controls following re-watering.

Effects on flowering time. In addition to the enhanced tolerance to abiotic stress, overexpression lines for G3086 or G592 show a very marked acceleration in the onset of flowering. Reflecting this rapid progression through the life cycle, overexpression lines for either gene tend to have a rather spindly appearance and reduced size compared to controls.

Tables 8-17 shows a number of polypeptides of the invention and include the amino acid residue coordinates for the conserved domains, the conserved domain sequences of the respective polypeptides, (sixth column); the identity in percentage terms to the conserved domain of the lead Arabidopsis sequence (the first transcription factor listed in each table), and whether the given sequence in each row was shown to confer increased biomass and yield or stress tolerance in plants (+) or has thus far not been shown to confer stress tolerance (−) for each given promoter::gene combination in our experiments. Percentage identities to the sequences listed in Tables 8-17 were determined using BLASTP analysis with defaults of wordlength (W) of 3, an expectation (E) of 10, and the BLOSUM62 scoring matrix Henikoff & Henikoff (1989) Proc. Natl. Acad. Sci. USA 89:10915).

TABLE 8

Conserved domains of G481 and closely related sequences

| | | | | % ID to | |
|---|---|---|---|---|---|
| | Species/GID | | | CCAAT-box | |
| Polypeptide | No., Accession | Domain | | binding | Abiotic |
| SEQ ID | No., or | Amino Acid | | conserved | Stress |
| NO: | Identifier | Coordinates | B Domain | domain of G481 | Tolerance |
| 2 | At/G481 | 20-110 | REQDRYLPIANISRIMKKALPPNGKI | 100% | + |
| | | | GKDAKDTVQECVSEFISFITSEASD | | |
| | | | KCQKEKRKTVNGDDLLWAMATLG | | |
| | | | FEDYLEPLKIYLARYRE | | |
| 4 | At/G3470 | 27-117 | REQDRYLPIANISPIMKKALPPNGKI | 93% | + |
| | | | AKDAKDTMQECVSEFISFITSEASE | | |
| | | | KCQKEKRKTINGDDLLWAMATLG | | |
| | | | FEDYIEPLKVYLARYRE | | |
| 6 | At/G3471 | 26-116 | REQDRYLPIANISRIMKKALPPNGKI | 93% | + |
| | | | AKDAKDTMQECVSEFISFITSEASE | | |
| | | | KCQKBKRKTINGDDLLWAMATLG | | |
| | | | FEDYIEPLKVYLARYRE | | |
| 8 | Zm/G3876 | 30-120 | REQDRFLPIANISRIMKKAIPANGKI | 87% | + |
| | | | AKDAKETVQECVSEFISFITSEASDK | | |
| | | | CQREKRKTINGDDLLWAMATLGFE | | |
| | | | DYIEPLKVYLQKYRE | | |
| 10 | At/G3394 | 38-127 | RQDRFLPIANISRIMKKAIPANGKIA | 87% | − |
| | | | KDAKETVQECVSEFISFITSEASDKC | | |
| | | | QREKRKTINGDDLLWAMATLGFED | | |
| | | | YIEPLKVYLQKYRE | | |
| 12 | Zm/G3434 | 18-108 | REQDRFLPIANISRIMKKAVPANGKI | 85% | + |
| | | | AKDAKETLQECVSEFISFVTSEASD | | |
| | | | KCQKEKRKTINGDDLLWAMATLG | | |
| | | | FEEYVEPLKIYLQKYKE | | |
| 14 | At/G1364 | 29-119 | REQDRFLPIANISRIMKRGLPANGKI | 85% | + |
| | | | AKDAKEIVQECVSEFISFVTSEASD | | |
| | | | KCQREKRKTINGDDLLWAMATLGF | | |
| | | | EDYMEPLKVYLMRYRE | | |
| 16 | Gm/G3475 | 23-113 | REQDRFLPIANVSRIMKKALPANAK | 84% | + |
| | | | ISKDAKETVQECVSEFISFITGEASD | | |
| | | | KCQREKRKTINGDDLLWAMTTLGF | | |
| | | | EDYVEPLKGYLQRFRE | | |
| 18 | At/G485 | 20-110 | REQDRFLPIANVSRIMKKALPANAK | 84% | + |
| | | | ISKDAKETVQECVSEFISFITGEASD | | |
| | | | KCQRFKRKTINGDDLLWAMTTLGF | | |
| | | | EDYVEPLKVYLQKYRE | | |
| 20 | Gm/G3476 | 26-116 | REQDRFLPIANVSRIMKKALPANAK | 84% | + |
| | | | ISKDAKETVQECVSEFISFITGEASD | | |
| | | | KCQREKRKTINGDDLLWAMTTLGF | | |
| | | | EEYVEPLKIYLQRFRE | | |
| 22 | At/G2345 | 28-118 | REQDRFLPIANISRIMKRGLPLNGKI | 84% | + |
| | | | AKDAKETMQECVSEFISFVTSEASD | | |
| | | | KCQREKRKTINGDDLLWAMATLGF | | |
| | | | EDYIDPLKVYLMRYRE | | |
| 24 | Gm/G3474 | 25-115 | REQDRFLPIANVSRIMKKALPANAK | 84% | − |
| | | | ISKEAKETVQECVSEFISFITGEASD | | |
| | | | KCQKEKRKTINGDDLLWAMTTLGF | | |
| | | | EDYVDPLKIYLHKYRE | | |
| 26 | Gm/G3478 | 23-113 | REQDRFLPIANVSRIMKKALPANAK | 84% | − |
| | | | ISKDAKETVQECVSEFISFITGEASD | | |
| | | | KCQREKRKTINGDDLLWAMTTLGF | | |
| | | | EDYVEPLKGYLQRFRE | | |
| 28 | At/G482 | 26-116 | REQDRFLPIANVSRIMKKALPANAK | 83% | + |
| | | | ISKDAKETMQECVSEFISFVTGEAS | | |
| | | | DKGQKEKRKTINGDDLLWAMTTL | | |
| | | | GFEDYVEPLKVYLQRFRE | | |
| 30 | Zm/G3435 | 22-112 | REQDRFLPIANYSRIMKKALPANAK | 83% | + |
| | | | ISKDAKETVQECVSEFISFITGEASD | | |
| | | | KCQREKRKTINGDDLLWAMTTLGF | | |
| | | | EDYVEPLKHYLHKFRE | | |
| 32 | Gm/G3472 | 25-115 | REQDRFLPIANVSRIMKKALPANAK | 83% | + |
| | | | ISKEAKETVQECVSEFISFITGEASD | | |
| | | | KGQKEKRKTINGDDLLWAMTTLGF | | |
| | | | EEYVEPLKVYLHKYRE | | |
| 34 | Zm/G3436 | 20-110 | REQDRFLPIANVSRIMKKALPANAK | 83% | + |
| | | | ISKDAKETVQECVSEFISFITGEASD | | |
| | | | KCQREKRKTINGDDLLWAMTTLGF | | |
| | | | EDYVEPLKLYLHKFRE | | |
| 36 | Os/G3397 | 23-113 | REQDRFLPIANVSRIMKKALPANAK | 82% | + |
| | | | ISKDAKETVQECVSEFISFITGEASD | | |
| | | | KCQREKRKTINGDDLLWAMTTLGF | | |
| | | | EDYVDPLKHYLHKFRE | | |
| 38 | Os/G3395 | 19-109 | REQDRFLPIANSRIMKKAVPANGKI | 82% | + |
| | | | AKDAKETLQECVSEFISFVTSEASD | | |
| | | | KCQKEKRKTINGEDLLFAMGTLGF | | |
| | | | EEYVDPLKIYLHKYRE | | |
| 40 | Os/G3398 | 20-110 | REQDRFLPIANVSRIMKRALPANAK | 81% | + |
| | | | ISKDAKETVQECVSEFISFITGEASD | | |
| | | | KCQREKRKTINGDDLLWMATTLGF | | |
| | | | EDYIDPLKLYLHKFRE | | |
| 42 | Os/G3396 | 20-111 | KEQDRFLPIANIGRIMRRAVPENGKI | 78% | + |
| | | | AKDSKESVQECVSEFISFITSEASDK | | |
| | | | CLKEKRKTINGDDLIWSMGTLGFE | | |
| | | | DYVEPLKLYLRLYRE | | |
| 58 | Os/G3429 | 37-125 | TNAELPMANLVRLIKKVLPGKAKI | 43% | + |
| | | | GGAAKGLTHDCAVEFVGFVGDEAS | | |
| | | | EKAKAEHRRTVAPEDYLGSFGDLG | | |
| | | | FDRYVDPMDAYIHGYRE | | |

TABLE 9

Conserved domains of G682 and closely related sequences

| | | | | % ID to | | Altered | | |
|---|---|---|---|---|---|---|---|---|
| | Species/ | | | MYB- | | C/N | | Water |
| | GID No., | | | related | | Sensing | | deprivation |
| SEQ | Accession | Domain in | | conserved | | and/or | | or osmotic |
| ID | No., or | Amino Acid | MYB-related | domain of | Salt Stress | tolerance | Cold | stress |
| NO: | Identifier | Coordinates | Domain | G682 | Tolerance | to low N₂ | Tolerance | tolerance |
| 60 | At/G682 | 33-77 | VNMSQEEEDLVS | 100% | + | + | + | + |
| | | | RMHKLVGDRWE | | | | | |
| | | | LIAGRIPGRTAGE | | | | | |
| | | | IERFWVMKN | | | | | |
| 62 | At/G226 | 38-82 | ISMTEQEEDLISR | 80% | − | + | + | + |
| | | | MYRLVGNRWDL | | | | | |
| | | | IAGRVVGRKANE | | | | | |
| | | | IERYWIMRN | | | | | |
| 64 | At/G2718 | 32-76 | IAMAQEEEDLICR | 80% | − | + | − | + |
| | | | MYKLVGERWDL | | | | | |
| | | | IAGRIPGRTAEEIE | | | | | |
| | | | RFWVMKN | | | | | |
| 66 | Os/G3393 | 31-75 | VHFTEEEEDLVF | 71% | − | + | + | + |
| | | | RMHRLVGNRWE | | | | | |
| | | | LIAGRIPGRTAKE | | | | | |
| | | | VEMFWAVKH | | | | | |
| 68 | Zm/G3431 | 31-75 | VDFTEAEEDLVS | 70% | − | + | + | + |
| | | | RMHRLVGNRWE | | | | | |
| | | | IIAGRIPGRTAEE | | | | | |
| | | | VEMFWSKKY | | | | | |
| 70 | Zm/G3444 | 31-75 | VDFTEAEEDLVS | 70% | − | + | − | + |
| | | | RMHRLVGNRWE | | | | | |
| | | | IIAGRIPGRTAEE | | | | | |
| | | | VEMFWSKKY | | | | | |
| 72 | Os/G3392 | 32-76 | VHFTEEEEDIVFR | 68% | + | + | + | − |
| | | | MHRLVGNRWELI | | | | | |
| | | | AGRIPGRTAEEV | | | | | |
| | | | EKFWAIKH | | | | | |
| 74 | Gm/G3450 | 20-64 | IHMSEQEEDLIRR | /68% | + | + | + | + |
| | | | MYKLVGDKWNL | | | | | |
| | | | IAGRIPGRKAEEI | | | | | |
| | | | ERFWIMRH | | | | | |
| 76 | At/G1816 | 30-74 | INMTEQEEDLIFR | 64% | − | + | − | + |
| | | | MYRLVGDRWDL | | | | | |
| | | | IAGRVPGRQPEEI | | | | | |
| | | | ERYWIMRN | | | | | |
| 78 | Gm/G3449 | 26-70 | VEFSEDEETLIIR | 63% | − | + | + | − |
| | | | MYKLVGERWSLI | | | | | |
| | | | AGRIPGRTAEEIE | | | | | |
| | | | KYWTSRF | | | | | |
| 80 | Gm/G3448 | 26-70 | VEFSEDEETLIIR | 61% | − | + | + | + (1 line |
| | | | MYKLVGERWSII | | | | | only) |
| | | | AGRIPGRTAEEIE | | | | | |
| | | | KYWTSRF | | | | | |
| 82 | Gm/G3446 | 26-70 | VEFSEAEEILIAM | 56% | − | − | − | + (1 line |
| | | | VYNLVGERWSLI | | | | | only) |
| | | | AGRIPGRTAEEIE | | | | | |
| | | | KYWTSRF | | | | | |
| 84 | Gm/G3445 | 25-69 | VEFSEAEEILIAM | 56% | − | − | − | − |
| | | | VYNLVGERWSLI | | | | | |
| | | | AGRIPGRTAEEIE | | | | | |
| | | | KYWTSRF | | | | | |

TABLE 10

Conserved domains of G867 and closely related sequences

| | | AP2 and B3 | | % ID to | | | |
|---|---|---|---|---|---|---|---|
| SEQ | | Domains in | | G867 | | % ID to | Abiotic |
| ID | Species/ | AA | | AP2 | | G867 B3 | Stress |
| NO: | GID No. | Coordinates | AP2 Domain | Domain | B3 Domain | Domain | Tolerance |
| 88 | At/G867 | AP2 | SSKYKGVVPQPN | 100% | LFEKAVTPSDVGKLN | 100% | + |
| | | 59-124 | GRWGAQIYEKHQ | | RLVIPKHHAEKHFPL | | |
| | | B3 | RVWLGTFNEEDE | | PSSNVSVKGVLLNFE | | |
| | | 187-272 | AARAYDVAVHRF | | DVNGKVWRFRYSY | | |
| | | | RRRDAVTNFKDV | | WNSSQSYVLTKGWS | | |
| | | | KMDEDE | | RFVKEKNLRAGDVV | | |
| 90 | At/G993 | AP2 | SSKYKGVVPQPN | 89% | LFEKTVTPSDVGKLN | 79% | + |
| | | 69-134 | GRWGAQIYEKHQ | | RLVIPKQHAEKHFPL | | |
| | | B3 | RVWLGTFNEEEE | | PAMTTAMGMNPSPT | | |
| | | 194-286 | AASSYDIAVRRFR | | KGVLINLEDRTGKV | | |
| | | | GRDAVTNFKSQV | | WRFRYSYWNSSQSY | | |
| | | | DGNDA | | VLTKGWSRFVKEKN | | |
| | | | | | LRAGDVV | | |
| 92 | At/G1930 | AP2 | SSRFKGVVPQPNG | 86% | LFEKTVTPSDVGKLN | 87% | + |
| | | 59-124 | RWGAQIYEKHQR | | RLVIPKHQAEKHFPL | | |
| | | B3 | VWLGTFNEEDEA | | PLGNNNVSVKGMLL | | |
| | | 182-269 | ARAYDVAAHRFR | | NFEDVNGKVWRFRY | | |
| | | | GRDAVTNFKDTTF | | SYWNSSQSYVLTKG | | |
| | | | EEEV | | WSRFVKEKRLCAGD | | |
| | | | | | LI | | |
| 94 | Os/G3391 | AP2 | SSKFKGVYPQPNG | 84% | LFDKTVTPSDVGKLN | 83% | + |
| | | 79-145 | RWGAQIYERHQR | | RLVIPKQHAEKHFPL | | |
| | | B3 | VWLGTFAGEDDA | | QLPSAGGESKGVLLN | | |
| | | 215-302 | ARAYDVAAQRFR | | FEDAAGKVWRFRYS | | |
| | | | GRDAVTNFRPLAE | | YWNSSQSYVLTKGW | | |
| | | | ADPDA | | SRFVKEKGLHADGK | | |
| | | | | | L | | |
| 96 | Gm/G3455 | AP2 | SSKYKGVVPQPN | 83% | LFQKAVTPSDVGKLN | 81% | + |
| | | 74-139 | GRWGSQIYEKHQ | | RLVIPKQHAEKHFPL | | |
| | | B3 | RVWLGTFNEEDE | | QSAANGVSATATAA | | |
| | | 204-296 | AARAYDVAVQRF | | KGVLLNFEDVGGKV | | |
| | | | RGKDAVTNFKPLS | | WRFRYSYWNSSQSY | | |
| | | | GTDDD | | VLTKGWSRFVKEKN | | |
| | | | | | LKAGDTV | | |
| 98 | Gm/G3452 | AP2 | SSKYKGVVPQPN | 83% | LFEKTVTPSDVGKLN | 78% | + |
| | | 51-116 | GRWGAQIYEKHQ | | RLVIPKQHAEKHFPL | | |
| | | B3 | RVWLGTFNEEDE | | SGSGDESSPCVAGAS | | |
| | | 171-266 | AARAYDIAALRFR | | AAKGMLLNFEDVGG | | |
| | | | GPDAVTNFKPPAA | | KVWRFRYSYWNSSQ | | |
| | | | SDDA | | SYVLTKGWSRFVKE | | |
| | | | | | KNLRAGDAV | | |
| 100 | Gm/G3453 | AP2 | SSKYKGVVPQPN | 83% | LVEKTVTPSDVGKLN | 77% | + |
| | | 57-122, | GRWGAQIYEKHQ | | RLVIPKQHAEKRFPL | | |
| | | B3 | RVWLGTFNEEDE | | SGSGGGALPCMAAA | | |
| | | 177-272 | AVRAYDIVAHRFR | | AGAKGMLLNFEDVG | | |
| | | | GRDAVTNFKPLA | | GKVWRFRYSYWNSS | | |
| | | | GADDA | | QSYVLTKGWSRFVK | | |
| | | | | | EKNLRAGDAV | | |
| 102 | Zm/G3432 | AP2 | SSRYKGVVPQPNG | 82% | LFDKTVTPSDVGKLN | 82% | + |
| | | 75-141 | RWGAQIYERHQR | | RLVIPKQHAEKHFPL | | |
| | | B3 | VWLGTFAGEADA | | QLPSAGGESKGVLLN | | |
| | | 212-299 | ARAYDVAAQRFR | | LEDAAGKVWRFRYS | | |
| | | | GRDAVTNFRPLA | | YWNSSQSYVLTKGW | | |
| | | | DADPDA | | SRFVKEKGLQAGDV | | |
| | | | | | V | | |
| 104 | Os/G3389 | AP2 | SSRYKGVVPQPNG | 82% | LFEKAVTPSDVGKLN | 78% | + |
| | | 64-129 | RWGAQIYERHAR | | RLVVPKQQAERHFPF | | |
| | | B3 | VWLGTFPDEEAA | | PLRRHSSDAAGKGVL | | |
| | | 177-266 | ARAYDVAALRFR | | LNFEDGDGKVWRFR | | |
| | | | GRDAVTNRAPAA | | YSYWNSSQSYVLTK | | |
| | | | EGASA | | GWSRFVREKGLRPG | | |
| | | | | | DTV | | |
| 106 | At/G9 | AP2 | SSKYKGVVPQPN | 81% | LFEKAVTPSDVGKLN | 91% | + |
| | | 62-127 | GRWGAQIYEKHQ | | RLVIPKQHAEKHFPL | | |
| | | B3 | RVWLGTFNEQEE | | PSPSPAVTKGVLINFE | | |
| | | 187-273 | AARSYDIAACRFR | | DVNGKVWRFRYSY | | |
| | | | GRDAVVNFKNVL | | WNSSQSYVLTKGWS | | |
| | | | EDGDL | | RFVKEKNLRAGDVV | | |
| 108 | Gm/G3451 | AP2 | SSKYKGVVPQPN | 81% | LFEKAVTPSDVGKLN | 78% | + |
| | | 80-146 | GRWGAQIYEKHQ | | RLVIPKQHAEKHFPL | | |
| | | B3 | RVWLGTFNEEDE | | QSSNGVSATTIAAVT | | |
| | | 209-308 | AARAYDIAAQRFR | | ATPTAAKGVLLNFED | | |
| | | | GKDAVTNFKPLA | | VGGKVWRFRYSYW | | |
| | | | GADDDD | | NSSQSYVLTKGWSRF | | |
| | | | | | VKEKNLKAGDTV | | |
| 110 | Os/G3388 | AP2 | SSRYKGVVPQPNG | 78% | LFEKAVTPSDVGKLN | 76% | n/d |
| | | 66-131 | RWGAQIYERHAR | | RLVVPKQHAEKHFPL | | |
| | | B3 | VWLGTFPDEEAA | | RRAASSDSASAAATG | | |
| | | 181-274 | ARAYDVAALRYR | | KGVLLNFEDGEGKV | | |
| | | | GRDAATNFPGAA | | WRFRYSYWNSSQSY | | |
| | | | ASAAE | | VLTKGWSRFVREKG | | |
| | | | | | LRAGDTI | | |
| 112 | Os/G3390 | AP2 | SSKYKGVVPQPN | 77% | LFDKTVTPSDVGKLN | 70% | + |
| | | 66-131 | GRWGAQIYERHQ | | RLVIPKQHAEKHFPL | | |
| | | B3 | RVWLGTFTGEAE | | QLPPPTTTSSVAAAA | | |
| | | 192-294 | AARAYDVAAQRF | | DAAAGGGDCKGVLL | | |
| | | | RGRDAVTNFRPLA | | NFEDAAGKVWKFRY | | |
| | | | ESDPE | | SYWNSSQSYVLTKG | | |
| | | | | | WSRFVKEKGLHAGD | | |
| | | | | | AV | | |

TABLE 11

Conserved domains of G1073 and closely related sequences

| | | AT-hook | | | | | | |
|---|---|---|---|---|---|---|---|---|
| | | and Second | | | | | | |
| | | Conserved | | | | | | |
| | | Domains in | | | | % ID to | | |
| | | AA | | % ID to | | Second | | |
| SEQ | | Coordinates | | AT-hook | Second | Conserved | Water | |
| ID | | and Base | AT-hook | Domain | Conserved | Domain of | deprivation | Greater |
| NO: | GID No. | Coordinates | domain | of G1073 | Domain | G1073 | Tolerance | Biomass |
| 114 | At/G1073 | Polypeptide | RRPRGRPAG | 100% | VSTYATRRGC | 100% | + | + |
| | | coordinates | | | GVCIISGTGAV | | | |
| | | 63-71, | | | TNVTIRQPAAP | | | |
| | | 107-204 | | | AGGGVITLHGR | | | |
| | | | | | FDILSLTGTALP | | | |
| | | | | | PPAPPGAGGLT | | | |
| | | | | | VYLAGGQGQV | | | |
| | | | | | VGGNVAGSLIA | | | |
| | | | | | SGPVVLMAASF | | | |
| 116 | Os/G3406 | Polypeptide | RRPRGRPPG | 89% | VSTYARRRQR | 71% | * | − |
| | | coordinates: | | | GVCVLSGSGV | | | |
| | | 82-90, | | | VTNVTLRQPSA | | | |
| | | 126-222 | | | PAGAVVSLHG | | | |
| | | | | | RFEILSLSGSFL | | | |
| | | | | | PPPAPPGATSLT | | | |
| | | | | | IFLAGGQGQVV | | | |
| | | | | | GGNVVGALYA | | | |
| | | | | | AGPVIVIAASF | | | |
| 118 | Os/G3399 | Polypeptide | RRPRGRPPG | 89% | VAEYARRRGR | 71% | + | + |
| | | coordinates: | | | GVCVLSGGGA | | | |
| | | 99-107, | | | VVNVALRQPG | | | |
| | | 143-240 | | | ASPPGSMVATL | | | |
| | | | | | RGRFEILSLTGT | | | |
| | | | | | VLPPPAPPGAS | | | |
| | | | | | GLTVFLSGGQG | | | |
| | | | | | QVIGGSVVGPL | | | |
| | | | | | VAAGPVVLMA | | | |
| | | | | | AS | | | |
| 120 | At/G1067 | Polypeptide | KRPRGRPPG | 78% | VSTYARRRGR | 69% | + | − |
| | | coordinates: | | | GVSVLGGNGT | | | |
| | | 86-94, | | | VSNVTLRQPVT | | | |
| | | 130-235 | | | PGNGGGVSGG | | | |
| | | | | | GGVVTLHGRF | | | |
| | | | | | EILSLTGTVLPP | | | |
| | | | | | PAPPGAGGLSIF | | | |
| | | | | | LAGGQGQVVG | | | |
| | | | | | GSVVAPLIASA | | | |
| | | | | | PVILMAASF | 68% | * | + |
| 122 | Gm/G3459 | Polypeptide | RRPRGRPPG | 89% | VTAYARRRQR | | | |
| | | coordinates: | | | GICVLSGSGTV | | | |
| | | 76-84, | | | TNVSLRQPAAA | | | |
| | | 121-216 | | | GAVVTLHGRF | | | |
| | | | | | EILSLSGSFLPP | | | |
| | | | | | PAPPGATSLTIY | | | |
| | | | | | LAGGQGQVVG | | | |
| | | | | | GNVIGELTAAG | | | |
| | | | | | PVIVIAASF | | | |
| 124 | Os/G3400 | Polypeptide | RRPRGRPLG | 89% | VCEFARRRGR | 68% | + | + |
| | | coordinates: | | | GVSVLSGGGA | | | |
| | | 83-91, | | | VANVALRQPG | | | |
| | | 127-225 | | | ASPPGSLVATM | | | |
| | | | | | RGQFEILSLTGT | | | |
| | | | | | VLPPPAPPSAS | | | |
| | | | | | GLTVFLSGGQG | | | |
| | | | | | QVVGGSVAGQ | | | |
| | | | | | LLAAGPVFLMA | | | |
| | | | | | ASF | | | |
| 372 | At/G2789 | Polypeptide | RRPRGRPAG | 100% | LAVFARRRQR | 67% | * | − |
| | | coordinates: | | | GVCVLTGNGA | | | |
| | | 59-67; | | | VTNVTVRQPG | | | |
| | | 103-196 | | | GGVVSLHGRFE | | | |
| | | | | | ILSLSGSFLPPP | | | |
| | | | | | APPAASGLKVY | | | |
| | | | | | LAGGQGQVIG | | | |
| | | | | | GSVVGPLTASS | | | |
| | | | | | PVVVMAASF | | | |
| 126 | Gm/G3460 | Polypeptide | RRPRGRPSG | 89% | VTAYARRRQR | 67% | + | + |
| | | coordinates: | | | GICVLSGSGTV | | | |
| | | 74-82, | | | TNVSLRQPAAA | | | |
| | | 118-213 | | | GAVVRLHGRF | | | |
| | | | | | EILSLSGSFLPP | | | |
| | | | | | PAPPGATSLTIY | | | |
| | | | | | LAGGQGQVVG | | | |
| | | | | | GNVVGELTAA | | | |
| | | | | | GPVIVIAASF | | | |
| 128 | At/G1667 | Polypeptide | KRPRGRPA | 89% | LSDFARRKQRG | 66% | n/d | + |
| | | coordinates: | G | | LCILSANGCVT | | | |
| | | 53-61; | | | NVTLRQPASSG | | | |
| | | 97-192 | | | AIVTLHGRYEI | | | |
| | | | | | LSLLGSILPPPA | | | |
| | | | | | PLGITGLTIYLA | | | |
| | | | | | GPQGQVVGGG | | | |
| | | | | | VVGGLIASGPV | | | |
| | | | | | VLMAASF | | | |
| 130 | At/G2156 | Polypeptide | KRPRGRPPG | 78% | VTTYARRRGR | 65% | + | + |
| | | coordinates: | | | GVSILSGNGTV | | | |
| | | 72-80, | | | ANVSLRQPATT | | | |
| | | 116-220 | | | AAHGANGGTG | | | |
| | | | | | GVVALHGRFEI | | | |
| | | | | | LSLTGTVLPPP | | | |
| | | | | | APPGSGGLSIFL | | | |
| | | | | | SGVQGQVIGG | | | |
| | | | | | NVVAPLVASGP | | | |
| | | | | | VILMAASF | | | |
| 132 | Gm/G3456 | Polypeptide | RRPRGRPPG | 89% | VAQFARRRQR | 65% | + | + |
| | | coordinates: | | | GVSILSGSGTV | | | |
| | | 62-70, | | | VNVNLRQPTAP | | | |
| | | 106-201 | | | GAVMALHGRF | | | |
| | | | | | DILSLTGSFLPG | | | |
| | | | | | PSPPGATGLTIY | | | |
| | | | | | LAGGQGQIVG | | | |
| | | | | | GEVVGIPLVAA | | | |
| | | | | | GPVLVMAATF | | | |
| 134 | Os/G3407 | Polypeptide | RRPRGRPPG | 89% | LTAYARRRQR | 63% | * | + |
| | | coordinates: | | | GVCVLSAAGT | | | |
| | | 63-71, | | | VANVTLRQPQS | | | |
| | | 106-208 | | | AQPGPASPAVA | | | |
| | | | | | TLHGRFEILSLA | | | |
| | | | | | GSFLPPPAPPG | | | |
| | | | | | ATSLAAFLAGG | | | |
| | | | | | QGQVVGGSVA | | | |
| | | | | | GALIAAGPVVV | | | |
| | | | | | VAASF | | | |
| 136 | Os/G3401 | Polypeptide | RRPRGRPPG | 89% | IAHFARRRQRG | 63% | + | + |
| | | coordinates: | | | VCVLSGAGTV | | | |
| | | 35-43, | | | TDVALRQPAAP | | | |
| | | 79-174 | | | SAVVALRGRFE | | | |
| | | | | | ILSLTGTFLPGP | | | |
| | | | | | APPGSTGLTVY | | | |
| | | | | | LAGGQGQVVG | | | |
| | | | | | GSVVGTLTAA | | | |
| | | | | | GPVMVIASTF | | | |
| 138 | At/G2153 | Polypeptide | RRPRGRPPG | 100% | LATFARRRQRG | 62% | + | + |
| | | coordinates: | | | ICILSGNGTVA | | | |
| | | 80-88, | | | NVTLRQPSTAA | | | |
| | | 124-227 | | | VAAAPGGAAV | | | |
| | | | | | LALQGRFEILSL | | | |
| | | | | | TGSFLPGPAPP | | | |
| | | | | | GSTGLTIYLAG | | | |
| | | | | | GQGQVVGGSV | | | |
| | | | | | VGPLMAAGPV | | | |
| | | | | | MLIAATF | | | |
| 140 | At/G1069 | Polypeptide | RRPRGRPPG | 89% | IAHFSRRRQRG | 62% | n/d | + |
| | | coordinates: | | | VCVLSGTGSVA | | | |
| | | 67-75, | | | NVTLRQAAAP | | | |
| | | 111-206 | | | GGVVSLQGRFE | | | |
| | | | ILSLTGAFLPGP | | | | | |
| | | | | | SPPGSTGLTVY | | | |
| | | | | | LAGVQGQVVG | | | |
| | | | | | GSVVGPLLAIG | | | |
| | | | | | SVMVIAATF | | | |
| 142 | Os/G3556 | Polypeptide | RRPRGRPPG | 89% | IAGFSRRRQRG | 62% | + | + |
| | | coordinates: | | | VSVLSGSGAVT | | | |
| | | 45-53; | | | NVTLRQPAGT | | | |
| | | 89-185 | | | GAAAVALRGR | | | |
| | | | | | FEILSMSGAFLP | | | |
| | | | | | APAPPGATGLA | | | |
| | | | | | VYLAGGQGQV | | | |
| | | | | | VGGSVMGELIA | | | |
| | | | | | SGPVMVIAATF | | | |
| 144 | At/G2157 | 88-96, | RRPRGRPPG | 89% | LNAFARRRGR | 60% | + | + |
| | | 132-228 | | | GVSVLSGSGLV | | | |
| | | | | | TNVTLRQPAAS | | | |
| | | | | | GGVVSLRGQFE | | | |
| | | | | | ILSMCGAFLPT | | | |
| | | | | | SGSPAAAAGLT | | | |
| | | | | | IYLAGAQGQV | | | |
| | | | | | VGGGVAGPLIA | | | |
| | | | | | SGPVIVIAATF | | | |
| 146 | Os/G3408 | 83-89, | KKRRGRPPG | 56% | LARFSSRRNLG | 44% | + | + |
| | | 91-247 | | | ICVLAGTGAVA | | | |
| | | | | | NVSLRHPSPGV | | | |
| | | | | | PGSAPAAIVFH | | | |
| | | | | | GRYEILSLSATF | | | |
| | | | | | LPPAMSSVAPQ | | | |
| | | | | | AAVAAAGLSIS | | | |
| | | | | | LAGPHGQIVGG | | | |
| | | | | | AVAGPLYAAT | | | |
| | | | | | TVVVVAAAF | | | |

TABLE 12

Conserved domains of G28 and closely related sequences

| | Species/GID | | | | |
|---|---|---|---|---|---|
| | No., | | | | |
| | Accession | AP2 Domain | | % ID to | |
| SEQ ID | No., or | Amino Acid | | conserved | Disease |
| NO: | Identifier | Coordinates | AP2 Domain | domain of G28 | Resistance |
| 148 | At/G28 | 144-208 | KGKHYRGVRQRPWGKFAAEIRDPA | 100% | + |
| | | | KNGARVWLGTFETAFDAALAYDR | | |
| | | | AAFRMRGSRALLNFPLRV | | |
| 150 | Bo/G3659 | 130-194 | KGKHYRGVRQRPWGKFAAEIRDPA | 100% | + |
| | | | KNGARVWLGTFETAEDAALAYDR | | |
| | | | AAFRMRGSRALLNFPLRV | | |
| 152 | At/G1006 | 113-177 | KAKHYRGVRQRPWGKFAAEIRDPA | 96% | + |
| | | | KNGARVWLGTFETAEDAALAYDIA | | |
| | | | AFRMRGSRALLNEPLRV | | |
| 154 | Gm/G3717 | 130-194 | KGKHYRGVRQRPWGKFAAEIRDPA | 98% | + |
| | | | KNGARVWLGTFETAEDAALAYDR | | |
| | | | AAYRMRGSRALLNFPLRV | | |
| 156 | Gm/G3718 | 139-203 | KGKHYRGVRQRPWGKFAAEIRDPA | 96% | + |
| | | | KNGARVWLGTFETAEDAALAYDR | | |
| | | | AAYRMRGSRALLNFPLRI | | |
| 158 | Bo/G3660 | 119-183 | KGKHYRGVRQRPWGKFAAEIRDPA | 96% | + |
| | | | KKGAREWLGTFETAEDAALAYDR | | |
| | | | AAFRMRGSRALLNFPLRV | | |
| 160 | Os/G3848 | 149-213 | RGKHYRGVRQRPWGKFAAEIRDPA | 93% | n/d |
| | | | KNGARVWLGTFDTAEDAALAYDR | | |
| | | | AAYRMRGSRALLNFPLRI | | |
| 162 | Zm/G3661 | 126-190 | RGKHYRGVRQRPWGKFAAEIRDPA | 90% | n/d |
| | | | RNGARVWLGTYDTAEDAALAYDR | | |
| | | | AAYRMRGSRALLNFPLRI | | |
| 164 | Ta/G3864 | 127-191 | RGKHFRGVRQRPWGKFAAEIRDPA | 89% | n/d |
| | | | KNGARVWLGTFDSAEDAAVAYDR | | |
| | | | AAYRMRGSRALLNFPLRI | | |
| 166 | Zm/G3856 | 140-204 | RGKHYRGVRQRPWGKFAAEIRDPA | 89% | n/d |
| | | | KNGARVWLGTYDSAEDAAVAYDR | | |
| | | | AAYRMRGSRALLNFPLRI | | |
| 168 | Os/G3430 | 145-209 | RGKHYRGVRQRPWGKFAAEIRDPA | 89% | + |
| | | | KNGARVWLGTFDSAEEAAVAYDR | | |
| | | | AAYRMRGSRALLNFPLRI | | |
| 170 | Le/G3841 | 102-166 | KGRHYRGVRQRPWGKFAAEIRDPA | 84% | n/d |
| | | | KNGARVWLGTYETAEEAAIAYDK | | |
| | | | AAYRMRGSKAHLNFPHRI | | |
| 172 | At/G22 | 88-152 | KGMQYRGVRRRPWGKFAAEIRDP | 82% | n/d |
| | | | KKNGARVWLGTYETPEDAAVAYD | | |
| | | | RAAFQLRGSKAKLNFPHLI | | |

TABLE 13

Conserved domains of G47 and closely related sequences

| | Species/ | | | | | |
|---|---|---|---|---|---|---|
| | GID No., | AP2 | | % ID to | | |
| SEQ | Accession | Domain | | conserved | Abiotic | Water |
| ID | No., or | Amino Acid | | domain of | Stress | deprivation |
| NO: | Identifier | Coordinates | AP2 Domain | G47 | Tolerance | Tolerance |
| 174 | At/G47 | 10-75 | SQSKYKGIRRRKWGKWVSE | 100% | + | + |
| | | | IRVPGTRDRLWLGSFSTAEG | | | |
| | | | AAVAHDVAFFCLHQPDSLES | | | |
| | | | LNFPHLL | | | |
| 176 | At/G2133 | 10-77 | DQSKYKGIRRRKWGKWVSE | 89% | + | + |
| | | | IRVPGTRQRLWLGSFSTAEG | | | |
| | | | AAVAHDVAFYCLHRPSSLD | | | |
| | | | DESFNFPHLL | | | |
| 184 | Os/G3649 | 15-87 | EMMRYRGVRRRRWGKWVS | 79% | + | + |
| | | | EIRVPGTRERLWLGSYATAE | | | |
| | | | AAAVAHDAAVCLLRLGGGR | | | |
| | | | RAAAGGGGGLNFPARA | | | |
| 182 | Os/G3644 | 52-122 | ERCRYRGVRRRRWGKWVS | 72% | +¹ | * |
| | | | EIRVPGTRERLWLGSYATPE | | | |
| | | | AAAVAHDTAVYFLRGGAGD | | | |
| | | | GGGGGATLNFPERA | | | |
| 178 | Gm/G3643 | 13-78 | TNNKLKGVRRRKWGKWVS | 68% | + | + |
| | | | EIRVPGTQERLWLGTYATPE | | | |
| | | | AAAVAHDVAVYCLSRPSSL | | | |
| | | | DKLNFPETL | | | |
| 180 | Zm/G3650 | 75-139 | RRCRYRGVRRRAWGKWVS | 65% | − | − |
| | | | EIRVPGTRERLWLGSYAAPE | | | |
| | | | AAAVAHDAAACLLRGCAGR | | | |
| | | | RLNFPGRAA | | | |

TABLE 14

Conserved domains of G1274 and closely related sequences

| | Species/ | | | | | |
|---|---|---|---|---|---|---|
| | GID No., | | | % ID to | | |
| SEQ | Accession | Domain | | conserved | Abiotic | |
| ID | No., or | Amino Acid | | domain of | Stress | Altered C/N |
| NO: | Identifier | Coordinates | WRKY Domain | G1274 | Tolerance | Sensing |
| 186 | At/G1274 | 110-166 | DDGFKWRKYGKKSVKNNINKRNYY | 100% | + | + |
| | | | KCSSEGCSVKKRVERDGDDAAYVIT | | | |
| | | | TYEGVHNH | | | |
| 188 | Gm/G3724 | 107-163 | DDGYKWRKYGKKSVKSSPNLRNYY | 84% | + | − |
| | | | KCSSGGCSVKKRVERDRDDYSYVIT | | | |
| | | | TYEGVHNH | | | |
| 190 | Zm/G3728 | 108-164 | DDGFKWRKYGKKAVKNSPNPRNYY | 82% | − | − |
| | | | RCSSEGCGVKKRVERDRDDPRYVIT | | | |
| | | | TYDGVHNH | | | |
| 192 | Zm/G3804 | 108-164 | DDGFKWRKYGKKAVKNSPNPRNYY | 82% | + | − |
| | | | RCSSEGCGVKKRVERDRDDPRYVIT | | | |
| | | | TYDGVHNH | | | |
| 194 | Gm/G3803 | 111-167 | DDGYKWRKYGKKTVKNNPNPRNYY | 80% | + | − |
| | | | KCSGEGCNVKKRVERDRDDSNYVLT | | | |
| | | | TYDGVHNH | | | |
| 196 | Zm/G3727 | 102-158 | DDGFKWRKYGKKAVKSSPNPRNYY | 80% | n/d | + |
| | | | RCSSEGCGVKKRVERDRDDPRYVIT | | | |
| | | | TYDGVHNH | | | |
| 198 | Os/G3721 | 96-152 | DDGFKWRKYGKKAVKNSPNPRNYY | 78% | + | − |
| | | | RCSTEGCNVKKRVERDREDHRYVIT | | | |
| | | | TYDGVHNH | | | |
| 200 | Zm/G3722 | 129-185 | DDGYKWRKYGKKSVKNSPNPRNYY | 78% | + | + |
| | | | RCSTEGCNVKKRVERDRDDPRYVVT | | | |
| | | | MYEGVHNH | | | |
| 202 | Os/G3726 | 135-191 | DDGYKWRKYGKKSVKNSPNPRNYY | 78% | + | − |
| | | | RCSTEGCNVKKRVERDKDDPSYVVT | | | |
| | | | TYEGTHNH | | | |
| 204 | Zm/G3720 | 135-191 | DDGYKWRKYGKKSVKNSPNPRNYY | 78% | n/d | n/d |
| | | | RCSTEGCNVKKRVERDKDDPSYVVT | | | |
| | | | TYEGMHNH | | | |
| 206 | Gm/G3723 | 112-168 | DDGYKWRKYGKKTVKSSPNPRNYY | 77% | − | − |
| | | | KCSGEGCDVKKRVERDRDDSNYVLT | | | |
| | | | TYDGVHNH | | | |
| 208 | At/G1275 | 113-169 | DDGFKWRKYGKKMVKNSPHPRNYY | 77% | + | − |
| | | | KCSVDGCPVKKRVERDRDDPSFVITT | | | |
| | | | YEGSHNH | | | |
| 210 | Os/G3730 | 107-163 | DDGFKWRKYGKKAVKSSPNPRNYY | 77% | n/d | − |
| | | | RCSAAGCGVKKRVERDGDDPRYVV | | | |
| | | | TTYDGVHNH | | | |
| 212 | Zm/G3719 | 98-154 | DDGFKWRKYGKKTVKSSPNPRNYY | 77% | n/d | − |
| | | | RCSAEGCGVKKRVERDSDDPRYVVT | | | |
| | | | TYDGVHNH | | | |
| 214 | Os/G3725 | 158-214 | DDGYKWRKYGKKSVKNSPNPRNYY | 75% | + | − |
| | | | RCSTEGGNYKKRVERDKNDPRYVVT | | | |
| | | | MYEGIHNH | | | |
| 216 | Os/G3729 | 137-193 | DDGYRWRKYGKKMVKNSPNPRNY | 75% | + | + |
| | | | YRCSSEGCRVKKRVERARDDARFVV | | | |
| | | | TTYDGVHNH | | | |

TABLE 15

Conserved domains of G1792 and closely related sequences

| | | AP2 and | | | | | | |
|---|---|---|---|---|---|---|---|---|
| | | EDLL | | % ID to | | % ID to | | |
| | | Domains in | | AP2 | | EDLL | Abiotic | |
| SEQ ID | GID No./ | aa | | Domain of | EDLL | Domain | stress | Disease |
| NO: | Species | Coordinates | AP2 domain | G1792 | Domain | of G1792 | tolerant | resistant |
| 222 | At/G1792 | 16-80; | KQARFRGVRRRPWGK | 100% | VFEFEYL | 100% | + | + |
| | | 117-132 | FAAEIRDPSRNGARL | | DDKVLEE | | | |
| | | | WLGTFETAEEAARAY | | LL | | | |
| | | | DRAAFNLRGHLAILNF | | | | | |
| | | | PNEY | | | | | |
| 224 | At/G1795 | 11-75; | EHGKYRGVRRRPWG | 69% | VFEFEYL | 93% | + | + |
| | | 104-119 | KYAAEIRDSRKHGER | | DDSVLEE | | | |
| | | | VWLGTFDTAEEAARA | | LL | | | |
| | | | YDQAAYSMRGQAAIL | | | | | |
| | | | NFPHEY | | | | | |
| 226 | At/G30 | 16-80; | EQGKYRGVRRRPWG | 70% | VFEFEYL | 87% | + | + |
| | | 100-115 | KYAAEIRDSRKHGER | | DDSVLDE | | | |
| | | | VWLGTFDTAEDAARA | | LL | | | |
| | | | YDRAAYSMRGKAAIL | | | | | |
| | | | NFPHEY | | | | | |
| 228 | Os/G3383 | 9-73; | TATKYRGVRRRPWGK | 79% | KIEFEYLD | 85% | + | n/d |
| | | 101-116 | FAAEIRDPERGGARV | | DKVLDDL | | | |
| | | | WLGTFDTAEEAARAY | | L | | | |
| | | | DRAAYAQRGAAAVL | | | | | |
| | | | NFPAAA | | | | | |
| 230 | At/G1791 | 10-74; | NEMKYRGVRKRPWG | 73% | VIEFEYLD | 81% | + | + |
| | | 108-123 | KYAAEIRDSARHGAR | | DSLLEELL | | | |
| | | | VWLGTFNTAEDAARA | | | | | |
| | | | YDRAAFGMRGQRAIL | | | | | |
| | | | NFPHEY | | | | | |
| 232 | Gm/G3519 | 13-77; | CEVRYRGIRRRPWGK | 78% | TFELEYLD | 80% | + | n/d |
| | | 128-143 | FAAEIRDPTRKGTRIW | | NKLLEEL | | | |
| | | | LGTFDTAEQAARAYD | | L | | | |
| | | | AAAFHFRGHRAILNFP | | | | | |
| | | | NEY | | | | | |
| 234 | Os/G3381 | 14-78; | LVAKYRGVRRRPWG | 76% | PIEFEYLD | 78% | + | + |
| | | 109-124 | KFAAEIRDSSRHGVRV | | DHVLQEM | | | |
| | | | WLGTFDTAEEAARAY | | L | | | |
| | | | DRSAYSMRGANAVLN | | | | | |
| | | | FPADA | | | | | |
| 236 | Os/G3737 | 8-72; | AASKYRGVRRRPWG | 76% | KVELVYL | 78% | + | n/d |
| | | 101-116 | KFAAEIRDPERGGSRV | | DDKVLDE | | | |
| | | | WLGTFDTAEEAARAY | | LL | | | |
| | | | DRAAFAMKGAMAVL | | | | | |
| | | | NFPGRT | | | | | |
| 238 | Os/G3515 | 11-75; | SSSSYRGVRKRPWGK | 75% | KVELECL | 78% | + | − |
| | | 116-131 | FAAEIRDPERGGARV | | DDKVLED | | | |
| | | | WLGTFDTAEEAARAY | | LL | | | |
| | | | DRAAFAMKGATAML | | | | | |
| | | | NFPGDH | | | | | |
| 240 | Zm/G3516 | 6-70; | KEGKYRGVRKRPWG | 74% | KVELECL | 78% | + | + |
| | | 107-122 | KFAAEIRDPERGGSRV | | DDRVLEE | | | |
| | | | WLGTFDTAEEAARAY | | LL | | | |
| | | | DRAAFAMKGATAVL | | | | | |
| | | | NFPASG | | | | | |
| 242 | Gm/G3520 | 14-78; | EEPRYRGVRRRPWGK | 80% | VIEFECLD | 75% | − | + |
| | | 109-124 | FAAEIRDPARHGARV | | DKLLEDL | | | |
| | | | WLGTFLTAEEAARAY | | L | | | |
| | | | DRAAYEMRGALAVL | | | | | |
| | | | NFPNEY | | | | | |
| 244 | Zm/G3517 | 13-77; | EPTKYRGVRRRPWGK | 72% | VIEFEYLD | 75% | + | + |
| | | 103-118 | YAAEIRDSSRHGVRIW | | DEVLQEM | | | |
| | | | LGTFDTAEEAARAYD | | L | | | |
| | | | RSANSMRGANAVLNF | | | | | |
| | | | PEDA | | | | | |
| 246 | Gm/G3518 | 13-77; | VEVRYRGIRRRPWGK | 78% | TFELEYFD | 73% | + | n/d |
| | | 135-150 | FAAEIRDPTRKGTRIW | | NKLLEEL | | | |
| | | | LGTFDTAEQAARAYD | | L | | | |
| | | | AAAFHFRGHRAILNFP | | | | | |
| | | | NEY | | | | | |
| 248 | Zm/G3739 | 13-77; | EPTKYRGVRRRPWGK | 72% | VIELEYLD | 68% | + | n/d |
| | | 107-122 | YAAEIRDSSRHGVRIW | | DEVLQEM | | | |
| | | | LGTFDTAEEAARAYD | | L | | | |
| | | | RSAYSMRGANAVLNF | | | | | |
| | | | PEDA | | | | | |
| 250 | Os/G3380 | 18-82; | ETTKYRGVRRRPSGK | 77% | VIELECLD | 62% | + | − |
| | | 103-118 | FAAEIRDSSRQSVRVW | | DQVLQEM | | | |
| | | | LGTFDTAEEAARAYD | | L | | | |
| | | | RAAYAMRGHLAVLN | | | | | |
| | | | FPAEA | | | | | |
| 252 | Zm/G3794 | 6-70; | EPTKYRGVRRRPSGKY | 73% | VIELECLD | 62% | + | n/d |
| | | 102-117 | AAEIRDSSRQSVRMW | | DQVLQEM | | | |
| | | | LGTFDTAEEAARAYD | | L | | | |
| | | | RAAYAMRGQIAVLNF | | | | | |
| | | | PAEA | | | | | |

TABLE 16

Conserved domains of G2999 and closely related sequences

| | | First and | | | | | |
|---|---|---|---|---|---|---|---|
| | | Second | | % ID to | | % ID to | |
| SEQ | | Domains in | | G2999 | | G2999 | Abiotic |
| ID | | AA | | First | | Second | Stress |
| No: | GID No. | Coordinates | ZF Domain | Domain | HD Domain | Domain | Tolerance |
| 256 | At/G2999 | 80-133; | ARYRECQKNHAAS | 100% | KKRFRTKFNEEQK | 100% | + |
| | | 198-261 | SGGHVVDGCGEFM | | EKMMEFAEKIGW | | |
| | | | SSGEEGTVESLLCA | | RMTKLEDDEVNR | | |
| | | | ACDCHRSFHRKEID | | FCREIKVKRQVFK | | |
| | | | | | VWMHNNKQAAK | | |
| | | | | | KKD | | |
| 258 | At/G2998 | 74-127, | VRYRECLKNHAAS | 81% | KKRFRTKFTTDQK | 72% | − |
| | | 240-303 | VGGSVHDGCGEFM | | ERMMDFAEKLGW | | |
| | | | PSGEEGTIEALRCA | | RMNKQDEEELKR | | |
| | | | ACDCHRNFHRKEM | | FCGEIGVKRQVFK | | |
| | | | D | | VWMHNNKNNAK | | |
| | | | | | KPP | | |
| 260 | At/G3000 | 58-111; | AKYRECQKNHAAS | 79% | KKRVRTKINEEQK | 65% | − |
| | | 181-244 | TGGHVVDGCCEFM | | EKMKEFAERLGW | | |
| | | | AGGEEGTLGALKC | | RMQKKDEEEIDKF | | |
| | | | AACNCHRSFHRKE | | CRMVNLRRQVFK | | |
| | | | VY | | VWMHNNKQAMK | | |
| | | | | | RNN | | |
| 262 | Os/G3690 | 161-213, | WRYRECLKNHAAR | 70% | KKRFRTKFTAEQK | 59% | + |
| | | 318-381 | MGAHVLDGCGEF | | ERMREFAHRVGW | | |
| | | | MSSPGDGAAALAC | | RIHKPDAAAVDAF | | |
| | | | AACGCHRSFHRREP | | CAQVGVSRRVLK | | |
| | | | A | | VWMHNNKHLAK | | |
| | | | | | TPP | | |
| 264 | At/G2997 | 47-100, | IRYRECLKNHAVNI | 69% | TKRFRTKFTAEQK | 61% | + |
| | | 157-220 | GGHAVDGCCEFMP | | EKMLAFAERLGW | | |
| | | | SGEDGTLDALKCA | | RIQKHDDVAVEQF | | |
| | | | ACGCHRNFHRKET | | CAETGVRRQVLKI | | |
| | | | E | | WMHNNKNSLGKK | | |
| | | | | | P | | |
| 266 | Zm/G3676 | 40-89; | ARYHECLRNHAAA | 69% | RKRFRTKFTPEQK | 57% | + |
| | | 162-255 | LGGHVVDGCGEFM | | EQMLAFAERLGW | | |
| | | | PGDGDSLKCAACG | | RLQKQDDALVQH | | |
| | | | CHRSFHRKDDA | | FCDQVGVRRQVF | | |
| | | | | | KVWMHNNKHTG | | |
| | | | | | RRQQ | | |
| 268 | Os/G3686 | 38-88; | CRYHECLRNHAAA | 68% | RRRSRTTFTREQK | 50% | + |
| | | 159-222 | SGGHVVDGCGEFM | | EQMLAFAERVGW | | |
| | | | PASTEEPLACAACG | | RIQRQEEATVEHF | | |
| | | | CHRSFHRRDPS | | CAQVGVRRQALK | | |
| | | | | | VWMHNNKHSFKQ | | |
| | | | | | KQ | | |
| 270 | At/G2996 | 73-126, | FRFRECLKNQAVNI | 67% | RKRHRTKFTAEQK | 54% | + |
| | | 191-254 | GGHAVDGCGEFMP | | ERMLALAERIGWR | | |
| | | | AGIEGTIDALKCAA | | IQRQDDEVIQRFC | | |
| | | | CGCHRNFHRKELP | | QETGVPRQVLKV | | |
| | | | | | WLHNNKHTLGKS | | |
| | | | | | P | | |
| 272 | At/G3001 | 62-113, | PHYYEGRKNHAAD | 63% | VKRLKTKFTAEQT | 48% | − |
| | | 179-242 | IGTTAYDGCGEFVS | | EKMRDYAEKLRW | | |
| | | | STGEEDSLNCAACG | | KVRPERQEEVEEF | | |
| | | | CHRNFHREELI | | CVEIGVNRKNFRI | | |
| | | | | | WMNNHKDKIIIDE | | |
| 274 | Os/G3685 | 43-95, | VRYHECLRNHAAA | 62% | RKRFRTKFTPEQK | 61% | + |
| | | 172-235 | MGGHVVDGCREF | | EQMLAFAERVGW | | |
| | | | MPMPGDAADALKC | | RMQKQDEALVEQ | | |
| | | | AACGCHRSFHRKD | | FCAQVGVRRQVF | | |
| | | | DG | | KVWMHNNKSSIG | | |
| | | | | | SSS | | |
| 276 | At/G2993 | 85-138, | IKYKECLKNHAAT | 62% | KKRFRTKFTQEQK | 58% | − |
| | | 222-285 | MGGNAIDGCGEFM | | EKMISFAERVGWK | | |
| | | | PSGEEGSIEALTCSV | | IQRQEESVVQQLC | | |
| | | | CNCHRNFHRRETE | | QEIGIRRRVLKVW | | |
| | | | | | MHNNKQNLSKKS | | |
| 278 | Zm/G3681 | 22-77; | PLYRECLKNHAASL | 62% | RKRFRTKFTAEQK | 54% | + |
| | | 208-271 | GGHAVDGCGEFMP | | QRMQELSERLGW | | |
| | | | SPGANPADPTSLKC | | RLQKRDEAVVDE | | |
| | | | AACGCHRNFHRRT | | WCRDMGVGKGVF | | |
| | | | V | | KVWMHNNKHNFL | | |
| | | | | | GGH | | |
| 280 | At/G2989 | 50-105; | VTYKECLKNHAAA | 61% | RKRFRTKFSSNQK | 62% | + |
| | | 192-255 | IGGHALDGCGEFM | | EKMHEFADRIGW | | |
| | | | PSPSSTPSDPTSLKC | | KIQKRDEDEVRDF | | |
| | | | AACGCHRNFHRRE | | CREIGVDKGVLKV | | |
| | | | TD | | WMHNNKNSFKFS | | |
| | | | | | G | | |
| 282 | At/G2991 | 54-109; | ATYKECLKNHAAG | 60% | RKRFRTKFSQYQK | 66% | − |
| | | 179-242 | IGGHALDGCGEFM | | EKMFEFSERVGW | | |
| | | | PSPSFNSNDPASLTC | | RMPKADDVVVKE | | |
| | | | AACGCHRNFHRRE | | FCREIGVDKSVFK | | |
| | | | ED | | VWMHNNKISGRS | | |
| | | | | | GA | | |
| 284 | At/G2990 | 54-109; | FTYKECLKNHAAA | 59% | RKRFRTKFSQFQK | 57% | + |
| | | 200-263 | LGGHALDGCGEFM | | EKMHEFAERVGW | | |
| | | | PSPSSISSDPTSLKC | | KMQKRDEDDVRD | | |
| | | | AACGCHRNFHRRD | | FCRQIGVDKSVLK | | |
| | | | PD | | VWMHNNLNTFNR | | |
| | | | | | RD | | |
| 286 | At/G2992 | 29-84, | VCYKECLKNHAAN | 59% | RKRTRTKFTPEQKI | 54% | + |
| | | 156-219 | LGGHALDGCGEFM | | KMRAFAEKAGWK | | |
| | | | PSPTATSTDPSSLRC | | INGCDEKSVREFC | | |
| | | | AACGCHRNFHRRD | | NEVGIERGVLKV | | |
| | | | PS | | WMHNNKYSLLNG | | |
| | | | | | K | | |
| 288 | At/G2995 | 3-58, | VLYNECLKNHAVS | 54% | KKHKRTKFTAEQ | 50% | + |
| | | 115-178 | LGGHALDGCGEFT | | KVKMRGFAERAG | | |
| | | | PKSTTILTDPPSLRC | | WKINGWDEKWVR | | |
| | | | DACGCHRNFHRRS | | EFCSEVGIERKVL | | |
| | | | PS | | KVWIHNNKYFNN | | |
| | | | | | GRS | | |
| 290 | At/G3002 | 5-53, | CVYRECMRNHAAK | 49% | QRRRKSKFTAFQR | 38% | + |
| | | 106-168 | LGSYAIDGCREYSQ | | EAMKDYAAKLG | | |
| | | | PSTGDLCVACGCH | | WTLKDKRALREEI | | |
| | | | RSYHRRIDV | | RVFCEGIGVTRYH | | |
| | | | | | FKTWVNNNKKFY | | |
| | | | | | H | | |

TABLE 17

Conserved domains of G3086 and closely related sequences

| | | | | | | |
|---|---|---|---|---|---|---|
| Species/ | | | | | | |
| | GID No., | | | % ID to | | |
| SEQ | Accession | Domain in | | conserved | Abiotic | |
| ID | No., or | Amino Acid | | domain of | Stress | Early |
| NO: | Identifier | Coordinates | bHLH Domain | G3086 | Tolerance | flowering |

| | | | | | | |
|---|---|---|---|---|---|---|
| 292 | At/G3086 | 307-365 | KRGCATHPRSIAERVRRTKIS | 100% | + | + |
| | | | ERMRKLQDLVPNMDTQTNT | | | |
| | | | ADMLDLAVQYIKDLQEQVK | | | |
| 294 | Gm/G3768 | 190-248 | KRGCATHPRSIAERVRRTKIS | 93% | + | + |
| | | | ERMRKLQDLVPNMDKQTNT | | | |
| | | | ADMLDLAVDYIKDLQKQVQ | | | |
| 296 | Gm/G3769 | 240-298 | KRGCATHPRSIAERVRRTKIS | 93% | + | + |
| | | | ERMRKLQDLVPNMDKQTNT | | | |
| | | | ADMLDLAVEYIKDLQNQVQ | | | |
| 298 | Gm/G3767 | 146-204 | KRGCATHPRSIAERVRRTKIS | 93% | + | + |
| | | | ERMRKLQDLVPNMDKQTNT | | | |
| | | | ADMLDLAVDYIKDLQKQVQ | | | |
| 300 | Os/G3744 | 71-129 | KRGCATHPRSIAERVRRTRIS | 89% | + | + |
| | | | ERIRKLQELVPNMDKQTNTA | | | |
| | | | DMLDLAVDYIKDLQKQVK | | | |
| 302 | Zm/G3755 | 97-155 | KRGCATHPRSIAERVRRTKIS | 89% | + | + |
| | | | ERIRKLQELVPNMDKQTNTS | | | |
| | | | DMLDLAVDYIKDLQKQVK | | | |
| 304 | Gm/G3766 | 35-93 | KRGCATHPRSIAERVRRTRIS | 88% | + | + |
| | | | ERMRKLQELVPHMDKQTNT | | | |
| | | | ADMLDLAVEYIKDLQKQFK | | | |
| 306 | At/G592 | 282-340 | KRGCATHPRSIAERVRRTRIS | 88% | −* | + |
| | | | ERMRKLQELVPNMDKQTNTS | | | |
| | | | DMLDLAVDYIKDLQRQYK | | | |
| 308 | Os/G3742 | 199-257 | KRGCATHPRSIAERVRRTRIS | 86% | n/d | n/d |
| | | | ERIRKLQELVPNMEKQTNTA | | | |
| | | | DMLDLAVDYIKELQKQVK | | | |
| 310 | Os/G3746 | 312-370 | KRGCATHPRSIAERERRTRIS | 79% | n/d | n/d |
| | | | KRLKKLQDLVPNMDKQTNTS | | | |
| | | | DMLDIAVTYIKELQGQVE | | | |
| 312 | Gm/G3771 | 84-142 | KRGCATHPRSIAERVRRTRIS | 79% | + | + |
| | | | DRIRKLQELVPNMDKQTNTA | | | |
| | | | DMLDEAVAYVKFLQKQIE | | | |
| 314 | Gm/G3765 | 147-205 | KRGFATHPRSIAERVRRTRISE | 79% | + | + |
| | | | RIRKLQELVPTMDKQTSTAE | | | |
| | | | MLDLALDYIKDLQKQFK | | | |
| 316 | At/G1134 | 187-245 | KRGCATHPRSIAERVRRTRIS | 77% | + | + |
| | | | DRIRKLQELVPNMDKQTNTA | | | |
| | | | DMLEEAVEYVKVLQRQIQ | | | |
| 318 | At/G2555 | 184-242 | KRGCATHPRSIAERVRRTRIS | 76% | + | + |
| | | | DRIRRLQELVPNMDKQTNTA | | | |
| | | | DMLEEAVEYVKALQSQIQ | | | |
| 320 | At/G2149 | 286-344 | KRGCATHPRSIAERERRTRIS | 74% | − | − |
| | | | GKLKKLQDLVPNMDKQTSYS | | | |
| | | | DMLDLAVQHIKGLQHQLQ | | | |
| 322 | At/G2766 | 234-292 | KRGFATHPRSIAERERRTRISG | 72% | + | + (1 line |
| | | | KLKKLQELVPNMDKQTSYAD | | | only) |
| | | | MLDLAVEHIKGLQHQVE | | | |
| 324 | Zm/G3760 | 243-300 | RRGQATDPHSIAERLRRERIA | 59% | + | + |
| | | | ERMKALQELVPNANKTDKAS | | | |
| | | | MLDEIVDYVKFLQLQVK | | | |
| 326 | Os/G3750 | 148-207 | RRGQATDPHSIAERLRRERIA | 57% | + | − |
| | | | ERMRALQELVPNTNKTDRAA | | | |

*data incomplete, soil drought assay not yet performed
¹two lines salt tolerant, but soil drought assay not yet performed
Abbreviations for Tables 8-17: At - Arabidopsis thaliana; Br - Brassica rapa subsp. Pekinensis, Bo- Brassica oleracea, Ca - Capsicum annuum; Gm - Glycine max; Ha - Helianthus annuus; Hv - Hordeum vulgare; La - Latuca sativa; Lc - Lotus corniculatus var. japonicus; Le - Lycopersicon esculentum; Mt - Medicago truncatula; Nt - Nicotiana tabacum; Os - Oryza sativa; St - Solanum tuberosum; Sb - Sorghum bicolor; Ta - Triticum aestivum; Ze - Zinnia elegans, Zm - Zea mays; + more tolerant than control plant in abiotic or disease assay n/d - assay not yet done

Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog or paralog, including equivalogs, may be identified by one or more of the methods described below.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994); Higgins et al. (1996)). Groups of similar genes can also be identified with pair-wise BLAST analysis (Feng and Doolittle (1987)). For example, a clade of very similar MADS domain transcription factors from Arabidopsis all share a common function in flowering time (Ratcliffe et al. (2001)), and a group of very similar AP2 domain transcription factors from Arabidopsis are involved in tolerance of plants to freezing (Gilmour et al. (1998)). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount (2001))

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994); Higgins et al. (1996)) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Transcription factor gene sequences are conserved across diverse eukaryotic species lines (Goodrich et al. (1993); Lin et al. (1991); Sadowski et al. (1988)). Plants are no exception to this observation; diverse plant species possess transcription factors that have similar sequences and functions.

Orthologous genes from different organisms have highly conserved functions, and very often essentially identical functions (Lee et al. (2002); Remm et al. (2001)). Paralogous genes, which have diverged through gene duplication, may retain similar functions of the encoded proteins. In such cases, paralogs can be used interchangeably with respect to certain embodiments of the instant invention (for example, transgenic expression of a coding sequence). An example of such highly related paralogs is the CBF family, with three well-defined members in Arabidopsis and at least one ortholog in Brassica napus, all of which control pathways involved in both freezing and drought stress (Gilmour et al. (1998); Jaglo et al. (2001)).

Distinct Arabidopsis transcription factors, including G28 (found in U.S. Pat. No. 6,664,446), G482 (found in US Patent Application 20040045049), G867 (found in US Patent Application 20040098764), and G1073 (found in U.S. Pat. No. 6,717,034), have been shown to confer stress tolerance or increased biomass when the sequences are overexpressed. The polypeptides sequences belong to distinct clades of transcription factor polypeptides that include members from diverse species. In each case, a significant number of clade member sequences derived from both dicots and monocots have been shown to confer increased biomass or tolerance to stress when the sequences were overexpressed (unpublished data). These references may serve to represent the many studies that demonstrate that conserved transcription factor genes from diverse species are likely to function similarly (i.e., regulate similar target sequences and control the same traits), and that transcription factors may be transformed into diverse species to confer or improve traits.

As shown in Tables 8-17, transcription factors that are phylogenetically related to the transcription factors of the invention may have conserved domains that share at least 38% amino acid sequence identity, and have similar functions.

At the nucleotide level, the sequences of the invention will typically share at least about 30% or 40% nucleotide sequence identity, preferably at least about 50%, about 60%, about 70% or about 80% sequence identity, and more preferably about 85%, about 90%, about 95% or about 97% or more sequence identity to one or more of the listed full-length sequences, or to a listed sequence but excluding or outside of the region(s) encoding a known consensus sequence or consensus DNA-binding site, or outside of the region(s) encoding one or all conserved domains. The degeneracy of the genetic code enables major variations in the nucleotide sequence of a polynucleotide while maintaining the amino acid sequence of the encoded protein.

Percent identity can be determined electronically, e.g., by using the MEGALIGN program (DNASTAR, Inc. Madison, Wis.). The MEGALIGN program can create alignments between two or more sequences according to different methods, for example, the clustal method (see, for example, Higgins and Sharp (1988) The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Software for performing BLAST analyses is publicly available, e.g., through the National Center for Biotechnology Information (see internet website at http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1993); Altschul et al. (1990)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). Unless otherwise indicated for comparisons of predicted polynucleotides, “sequence identity” refers to the % sequence identity generated from a tblastx using the NCBI version of the algorithm at the default settings using gapped alignments with the filter “off” (see, for example, internet website at http://www.ncbi.nlm.nih.gov/).

Other techniques for alignment are described by Doolittle (1996). Preferably, an alignment program that permits gaps in the sequence is utilized to align the sequences. The Smith-Waterman is one type of algorithm that permits gaps in sequence alignments (see Shpaer (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (see, for example, Hein (1990)) Identity between sequences can also be determined by other methods known in the art, e.g., by varying hybridization conditions (see US Patent Application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al. (1997)), PFAM, and other databases which contain previously identified and annotated motifs, sequences and gene functions. Methods that search for primary sequence patterns with secondary structure gap penalties (Smith et al. (1992)) as well as algorithms such as Basic Local Alignment Search Tool (BLAST; Altschul (1993); Altschul et al. (1990)), BLOCKS (Henikoff and Henikoff (1991)), Hidden Markov Models (HMM; Eddy (1996); Sonnhammer et al. (1997)), and the like, can be used to manipulate and analyze polynucleotide and polypeptide sequences encoded by polynucleotides. These databases, algorithms and other methods are well known in the art and are described in Ausubel et al. (1997), and in Meyers (1995).

A further method for identifying or confirming that specific homologous sequences control the same function is by comparison of the transcript profile(s) obtained upon overexpression or knockout of two or more related transcription factors. Since transcript profiles are diagnostic for specific cellular states, one skilled in the art will appreciate that genes that have a highly similar transcript profile (e.g., with greater than 50% regulated transcripts in common, or with greater than 70% regulated transcripts in common, or with greater than 90% regulated transcripts in common) will have highly similar functions. Fowler et al. (2002), have shown that three paralogous AP2 family genes (CBF1, CBF2 and CBF3), each of which is induced upon cold treatment, and each of which can condition improved freezing tolerance, have highly similar transcript profiles. Once a transcription factor has been shown to provide a specific function, its transcript profile becomes a diagnostic tool to determine whether paralogs or orthologs have the same function.

Furthermore, methods using manual alignment of sequences similar or homologous to one or more polynucleotide sequences or one or more polypeptides encoded by the polynucleotide sequences may be used to identify regions of similarity and AT-hook domains. Such manual methods are well-known of those of skill in the art and can include, for example, comparisons of tertiary structure between a polypeptide sequence encoded by a polynucleotide that comprises a known function and a polypeptide sequence encoded by a polynucleotide sequence that has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

Orthologs and paralogs of presently disclosed transcription factors may be cloned using compositions provided by the present invention according to methods well known in the art. cDNAs can be cloned using mRNA from a plant cell or tissue that expresses one of the present transcription factors. Appropriate mRNA sources may be identified by interrogating Northern blots with probes designed from the present transcription factor sequences, after which a library is prepared from the mRNA obtained from a positive cell or tissue. Transcription factor-encoding cDNA is then isolated using, for example, PCR, using primers designed from a presently disclosed transcription factor gene sequence, or by probing with a partial or complete cDNA or with one or more sets of degenerate probes based on the disclosed sequences. The cDNA library may be used to transform plant cells. Expression of the cDNAs of interest is detected using, for example, microarrays, Northern blots, quantitative PCR, or any other technique for monitoring changes in expression. Genomic clones may be isolated using similar techniques to those.

Examples of orthologs of the Arabidopsis polypeptide sequences and their functionally similar orthologs are listed in the Sequence Listing. In addition to the sequences in the Sequence Listing, the invention encompasses isolated nucleotide sequences that are phylogenetically and structurally similar to sequences listed in the Sequence Listing) and can function in a plant by increasing biomass, disease resistance and/or and abiotic stress tolerance when ectopically expressed in a plant. These polypeptide sequences represent transcription factors that show significant sequence similarity the polypeptides of the Sequence Listing particularly in their respective conserved domains, as identified in Tables 8-17.

Since a significant number of these sequences are phylogenetically and sequentially related to each other and have been shown to increase a plants biomass, disease resistance and/or abiotic stress tolerance, one skilled in the art would predict that other similar, phylogenetically related sequences falling within the present clades of transcription factors would also perform similar functions when ectopically expressed.

Identifying Polynucleotides or Nucleic Acids by Hybridization

Polynucleotides homologous to the sequences illustrated in the Sequence Listing and tables can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited below (e.g., Sambrook et al. (1989); Berger and Kimmel (1987); and Anderson and Young (1985)).

Encompassed by the invention are polynucleotide sequences that are capable of hybridizing to the claimed polynucleotide sequences, including any of the transcription factor polynucleotides within the Sequence Listing, and fragments thereof under various conditions of stringency (see, for example, Wahl and Berger (1987); and Kimmel (1987)). In addition to the nucleotide sequences listed in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al. (1989); Berger (1987), pages 467-469; and Anderson and Young (1985).

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

(I) DNA-DNA:

Tₘ(° C.)=81.5+16.6(log [Na+])+0.41(% G+C)−0.62(% formamide)−500/L

(II) DNA-RNA:

Tₘ(° C.)=79.8+18.5(log [Na+])+0.58(% G+C)+0.12(% G+C)²−0.5(% formamide)−820/L

(III) RNA-RNA:

Tₘ(° C.)=79.8+18.5(log [Na+])+0.58(% G+C)+0.12(% G+C)²−0.35(% formamide)−820/L

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young (1985)). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ−5° C. to Tₘ−20° C., moderate stringency at Tₘ−20° C. to Tₘ−35° C. and low stringency at Tₘ−35° C. to Tₘ−50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ−25° C. for DNA-DNA duplex and Tₘ−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or Northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present transcription factors include, for example:

6×SSC at 65° C.;

50% formamide, 4×SSC at 42° C.; or

0.5×SSC, 0.1% SDS at 65° C.;

with, for example, two wash steps of 10-30 minutes each. Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, US Patent Application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a transcription factor known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, including any of the transcription factor polynucleotides within the Sequence Listing, and fragments thereof under various conditions of stringency (see, for example, Wahl and Berger (1987), pages 399-407; and Kimmel (1987)). In addition to the nucleotide sequences in the Sequence Listing, fall length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

### EXAMPLES

It is to be understood that this invention is not limited to the particular devices, machines, materials and methods described. Although particular embodiments are described, equivalent embodiments may be used to practice the invention.

The invention, now being generally described, will be more readily understood by reference to the following examples, which are included merely for purposes of illustration of certain aspects and embodiments of the present invention and are not intended to limit the invention. It will be recognized by one of skill in the art that a transcription factor that is associated with a particular first trait may also be associated with at least one other, unrelated and inherent second trait which was not predicted by the first trait.

### Example I

### Project Types

A variety of constructs are being used to modulate the activity of lead transcription factors, and to test the activity of orthologs and paralogs in transgenic plant material. This platform provides the material for all subsequent analysis.

Transgenic lines from each particular transformation “project” are examined for morphological and physiological phenotypes. An individual project is defined as the analysis of lines for a particular construct or knockout (for example this might be 35S lines for a lead gene, 35S lines for a paralog or ortholog, lines for an RNAi construct, lines for a GAL4 fusion construct, lines in which expression is driven from a particular tissue specific promoter, etc.) In the current lead advancement program, four main areas of analysis were pursued, spanning a variety of different project types (e.g., promoter-gene combinations).

### (1) Overexpression/Tissue Specific/Conditional Expression

The promoters used in our experiments were selected in order to provide for a range of different expression patterns. Details of promoters being used, along with a characterization of the expression patterns that they produce are given in the Promoter Analysis (Example II).

Expression of a given TF from a particular promoter is achieved either by a direct-promoter fusion construct in which that TF is cloned directly behind the promoter of interest or by a two component system. Details of transformation vectors used in these studies are shown in the Vector and Cloning Information (Example III). A list of all constructs (PIDs) included in this report, indicating the promoter fragment that is being used to drive the transgene, along with the cloning vector backbone, is provided in the following Table. Compilations of the sequences of promoter fragments and the expressed transgene sequences within the PIDs are provided in the Sequence Listing.

TABLE 18
Sequences of promoter fragments and the expressed transgene sequences

| | | SEQ ID | | | | |
|---|---|---|---|---|---|---|
| | | NO: of | | | | |
| GID | PID | PID | Promoter | Project type | Promoter_ID | Vector |
| G9 | P167 | 421 | 35S | Direct promoter-fusion | N2 | pMEN20 |
| G9 | P7824 | 422 | opLexA | 2-components-supTfn (TF | N3 | P5480 |
| | | | | component of two-component | | |
| | | | | system) | | |
| G19 | P1 | 423 | 35S | Direct promoter-fusion | N2 | pMEN20 |
| G22 | P806 | 424 | 35S | Direct promoter-fusion | N2 | pMEN001 |
| G22 | P25649 | 425 | Prom-G22 | Promoter-reporter | N1146 | P21142 |
| G22 | P25648 | 426 | Prom-G22 | Promoter-reporter (YFP/LTI6b) | N1146 | P25755 |
| G28 | P21202 | 427 | 35S | Direct GR-fusion C-term | N2 | P21171 |
| G28 | P21277 | 428 | 35S | Direct GR-fusion HA C-term | N2 | P21172 |
| G28 | P21208 | 429 | 35S | Direct GR-fusion N-term | N2 | P21173 |
| G28 | P21283 | 430 | 35S | Direct GR-fusion HA N-term | N2 | P21174 |
| G28 | P21196 | 431 | 35S | GAL4 N-term | N2 | P21195 |
| G28 | P25444 | 432 | 35S | domain swap_1 | N2 | P21195 |
| G28 | P174 | 433 | 35S | Direct promoter-fusion | N2 | pMEN20 |
| G28 | P21143 | 434 | 35S | GAL4 C-term | N2 | P5425 |
| G28 | P25443 | 435 | 35S | deletion_2 | N2 | pMEN65 |
| G28 | P25678 | 436 | 35S | site-directed mutation_1 | N2 | pMEN65 |
| G28 | P25679 | 437 | 35S | site-directed mutation_2 | N2 | pMEN65 |
| G28 | P25680 | 438 | 35S | site-directed mutation_3 | N2 | pMEN65 |
| G28 | P25681 | 439 | 35S | site-directed mutation_4 | N2 | pMEN65 |
| G28 | P25682 | 440 | 35S | site-directed mutation_5 | N2 | pMEN65 |
| G28 | P25683 | 441 | 35S | site-directed mutation_6 | N2 | pMEN65 |
| G28 | P25684 | 442 | 35S | site-directed mutation_7 | N2 | pMEN65 |
| G28 | P25442 | 443 | 35S | deletion_1 | N2 | pMEN65 |
| G28 | P23541 | 444 | ARSK1 | Direct promoter-fusion | N1131 | pMEN65 |
| G28 | P23317 | 445 | ARSK1 | Direct promoter-fusion | N82 | pMEN65 |
| G28 | P23441 | 446 | CUT1 | Direct promoter-fusion | N19 | pMEN65 |
| G28 | P23543 | 447 | LTP1 | Direct promoter-fusion | N1135 | pMEN65 |
| G28 | P7826 | 448 | opLexA | 2-components-supTfn (TF | N3 | P5480 |
| | | | | component of two-component | | |
| | | | | system) | | |
| G28 | P25937 | 449 | opLexA | 2-components-supTfn-HA-C-term | N3 | P25461 |
| | | | | (TF component of two-component | | |
| | | | | system) | | |
| G28 | P26267 | 450 | opLexA | 2-components-supTfn-HA-N-term | N3 | P25976 |
| | | | | (TF component of two-component | | |
| | | | | system) | | |
| G28 | P21169 | 451 | Prom-G28 | Promoter-reporter | N517 | P21142 |
| G28 | P25712 | 452 | Prom-G28 | Promoter-reporter | N517 | P32122 |
| G28 | P25650 | 453 | Prom-G28 | Promoter-reporter (YFP/LTI6b) | N517 | P25755 |
| G28 | P23544 | 454 | RBCS3 | Direct promoter-fusion | N1136 | pMEN65 |
| G30 | P25086 | 455 | 35S | Direct GR-fusion C-term | N2 | P21171 |
| G30 | P25097 | 456 | 35S | Direct GR-fusion N-term | N2 | P21173 |
| G30 | P893 | 457 | 35S | Direct promoter-fusion | N2 | pMEN65 |
| G30 | P3852 | 458 | opLexA | 2-components-supTfn (TF | N3 | P5381 |
| | | | | component of two-component | | |
| | | | | system) | | |
| G30 | P25123 | 459 | Prom-G30 | Promoter-reporter | N1118 | P21142 |
| G47 | P25185 | 460 | 35S | Direct GR-fusion C-term | N2 | P21171 |
| G47 | P25187 | 461 | 35S | Direct GR-fusion N-term | N2 | P21173 |
| G47 | P25186 | 462 | 35S | GAL4 N-term | N2 | P21195 |
| G47 | P25184 | 463 | 35S | GAL4 C-term | N2 | P21378 |
| G47 | P25279 | 464 | 35S | Protein-GFP-C-fusion | N2 | P25799 |
| G47 | P894 | 465 | 35S | Direct promoter-fusion | N2 | pMEN65 |
| G47 | P25732 | 466 | 35S | site-directed mutation_1 | N2 | pMEN65 |
| G47 | P25733 | 467 | 35S | site-directed mutation_2 | N2 | pMEN65 |
| G47 | P25734 | 468 | 35S | site-directed mutation_3 | N2 | pMEN65 |
| G47 | P25735 | 469 | 35S | site-directed mutation_4 | N2 | pMEN65 |
| G47 | P25182 | 470 | 35S | domain swap_1 | N2 | pMEN65 |
| G47 | P3853 | 471 | opLexA | 2-components-supTfn (TF | N3 | P5381 |
| | | | | component of two-component | | |
| | | | | system) | | |
| G47 | P25195 | 472 | opLexA | 2-components-supTfn-TAP-C-term | N3 | P25420 |
| | | | | (TF component of two-component | | |
| | | | | system) | | |
| G47 | P25194 | 473 | opLexA | 2-components-supTfn-HA-C-term | N3 | P25461 |
| | | | | (TF component of two-component | | |
| | | | | system) | | |
| G47 | P26262 | 474 | opLexA | 2-components-supTfn-HA-N-term | N3 | P25976 |
| | | | | (TF component of two-component | | |
| | | | | system) | | |
| G47 | P25134 | 475 | Prom-G47 | Promoter-reporter | N1124 | P21142 |
| G47 | P25998 | 476 | Prom-G47 | Promoter-reporter (YFP/LTI6b) | N1124 | P25755 |
| G194 | P197 | 477 | 35S | Direct promoter-fusion | N2 | pMEN20 |
| G225 | P23525 | 478 | Prom-G225 | Promoter-reporter | N1112 | P21142 |
| G225 | P25137 | 479 | Prom-G225 | Promoter-reporter (YFP/LTI6b) | N1112 | P25755 |
| G226 | P3359 | 480 | opLexA | 2-components-supTfn (TF | N3 | P5480 |
| | | | | component of two-component | | |
| | | | | system) | | |
| G226 | P23526 | 481 | Prom-G226 | Promoter-reporter | N1113 | P21142 |
| G226 | P25138 | 482 | Prom-G226 | Promoter-reporter (YFP/LTI6b) | N1113 | P25755 |
| G481 | P21294 | 483 | 35S | RNAi (GS) | N2 | P21103 |
| G481 | P21300 | 484 | 35S | RNAi (clade) | N2 | P21103 |
| G481 | P21206 | 485 | 35S | Direct GR-fusion C-term | N2 | P21171 |
| G481 | P21281 | 486 | 35S | Direct GR-fusion HA C-term | N2 | P21172 |
| G481 | P21212 | 487 | 35S | Direct GR-fusion N-term | N2 | P21173 |
| G481 | P21287 | 488 | 35S | Direct GR-fusion HA N-term | N2 | P21174 |
| G481 | P21159 | 489 | 35S | RNAi (clade) | N2 | P21103 |
| G481 | P21305 | 490 | 35S | RNAi (clade) | N2 | P21103 |
| G481 | P21200 | 491 | 35S | GAL4 N-term | N2 | P21195 |
| G481 | P25281 | 492 | 35S | Protein-GFP-C-fusion | N2 | P25799 |
| G481 | P46 | 493 | 35S | Direct promoter-fusion | N2 | pMEN20 |
| G481 | P21146 | 494 | 35S | GAL4 C-term | N2 | P5425 |
| G481 | P21274 | 495 | 35S | TF dom neg deln 2ndry domain | N2 | pMEN65 |
| G481 | P21273 | 496 | 35S | TF dominant negative deletion | N2 | pMEN65 |
| G481 | P25885 | 497 | 35S | site-directed mutation_1 | N2 | pMEN65 |
| G481 | P25886 | 498 | 35S | site-directed mutation_2 | N2 | pMEN65 |
| G481 | P25888 | 499 | 35S | site-directed mutation_4 | N2 | pMEN65 |
| G481 | P25889 | 500 | 35S | site-directed mutation_5 | N2 | pMEN65 |
| G481 | P25890 | 501 | 35S | site-directed mutation_6 | N2 | pMEN65 |
| G481 | P26040 | 502 | 35S | Protein-CFP-C-fusion | N2 | P25801 |
| G481 | P25891 | 503 | 35S | domain swap_1 | N2 | pMEN65 |
| G481 | P25893 | 504 | 35S | splice_variant_1 | N2 | pMEN65 |
| G481 | P23325 | 505 | LTP1 | Direct promoter-fusion | N1141 | pMEN65 |
| G481 | P6812 | 506 | opLexA | 2-components-supTfn (TF | N3 | P5480 |
| | | | | component of two-component | | |
| | | | | system) | | |
| G481 | P25285 | 507 | opLexA | 2-components-supTfn-TAP-C-term | N3 | P25420 |
| | | | | (TF component of two-component | | |
| | | | | system) | | |
| G481 | P25455 | 508 | opLexA | 2-components-supTfn-HA-C-term | N3 | P25461 |
| | | | | (TF component of two-component | | |
| | | | | system) | | |
| G481 | P26263 | 509 | opLexA | 2-components-supTfn-HA-N-term | N3 | P25976 |
| | | | | (TF component of two-component | | |
| | | | | system) | | |
| G481 | P21167 | 510 | Prom-G481 | Promoter-reporter | N515 | P21142 |
| G481 | P25610 | 511 | Prom-G481 | Promoter-reporter | N515 | P32122 |
| G481 | P21522 | 512 | SUC2 | Direct promoter-fusion | N1142 | pMEN65 |
| G482 | P47 | 513 | 35S | Direct promoter-fusion | N2 | pMEN20 |
| G482 | P26041 | 514 | 35S | Protein-CFP-C-fusion | N2 | P25801 |
| G482 | P5072 | 515 | opLexA | 2-components-supTfn (TF | N3 | P5381 |
| | | | | component of two-component | | |
| | | | | system) | | |
| G483 | P48 | 516 | 35S | Direct promoter-fusion | N2 | pMEN20 |
| G483 | P26226 | 517 | 35S | Protein-YFP-C-fusion | N2 | P25800 |
| G484 | P26276 | 518 | 35S | Protein-CFP-C-fusion | N2 | P25801 |
| G485 | P1441 | 519 | 35S | Direct promoter-fusion | N2 | pMEN65 |
| G485 | P26044 | 520 | 35S | Protein-CFP-C-fusion | N2 | P25801 |
| G485 | P25892 | 521 | 35S | domain swap_1 | N2 | pMEN65 |
| G485 | P4190 | 522 | opLexA | 2-components-supTfn (TF | N3 | P5381 |
| | | | | component of two-component | | |
| | | | | system) | | |
| G489 | P51 | 523 | 35S | Direct promoter-fusion | N2 | pMEN20 |
| G489 | P26060 | 524 | 35S | Protein-YFP-C-fusion | N2 | P25800 |
| G489 | P3404 | 525 | opLexA | 2-components-supTfn (TF | N3 | P5381 |
| | | | | component of two-component | | |
| | | | | system) | | |
| G515 | P25421 | 526 | 35S | Direct promoter-fusion | N2 | pMEN65 |
| G516 | P279 | 527 | 35S | Direct promoter-fusion | N2 | pMEN20 |
| G517 | P2035 | 528 | 35S | Direct promoter-fusion | N2 | pMEN65 |
| G589 | P1042 | 529 | 35S | Direct promoter-fusion | N2 | pMEN20 |
| G591 | P77 | 530 | 35S | Direct promoter-fusion | N2 | pMEN20 |
| G592 | P310 | 531 | 35S | Direct promoter-fusion | N2 | pMEN20 |
| G592 | P25130 | 532 | Prom-G592 | Promoter-reporter | N1125 | P21142 |
| G592 | P25131 | 533 | Prom-G592 | Promoter-reporter (YFP/LTI6b) | N1125 | P25755 |
| G634 | P324 | 534 | 35S | Direct promoter-fusion | N2 | pMEN20 |
| G634 | P1374 | 535 | 35S | Direct promoter-fusion | N2 | pMEN65 |
| G634 | P1717 | 536 | 35S | Direct promoter-fusion | N2 | pMEN65 |
| G682 | P21299 | 537 | 35S | RNAi (clade) | N2 | P21103 |
| G682 | P21204 | 538 | 35S | Direct GR-fusion C-term | N2 | P21171 |
| G682 | P21279 | 539 | 35S | Direct GR-fusion HA C-term | N2 | P21172 |
| G682 | P23483 | 540 | 35S | Direct GR-fusion N-term | N2 | P21173 |
| G682 | P21111 | 541 | 35S | RNAi (GS) | N2 | P21103 |
| G682 | P23482 | 542 | 35S | GAL4 N-term | N2 | P21195 |
| G682 | P25290 | 543 | 35S | Protein-GFP-C-fusion | N2 | P25799 |
| G682 | P108 | 544 | 35S | Direct promoter-fusion | N2 | pMEN20 |
| G682 | P21144 | 545 | 35S | GAL4 C-term | N2 | P5425 |
| G682 | P23328 | 546 | LTP1 | Direct promoter-fusion | N1141 | pMEN65 |
| G682 | P5099 | 547 | opLexA | 2-components-supTfn (TF | N3 | P5381 |
| | | | | component of two-component | | |
| | | | | system) | | |
| G682 | P23516 | 548 | opLexA | 2-components-supTfn (TF | N3 | P5381 |
| | | | | component of two-component | | |
| | | | | system) | | |
| G682 | P23517 | 549 | opLexA | 2-components-supTfn (TF | N3 | P5381 |
| | | | | component of two-component | | |
| | | | | system) | | |
| G682 | P25656 | 550 | opLexA | 2-components-supTfn-TAP-C-term | N3 | P25420 |
| | | | | (TF component of two-component | | |
| | | | | system) | | |
| G682 | P25457 | 551 | opLexA | 2-components-supTfn-HA-C-term | N3 | P25461 |
| | | | | (TF component of two-component | | |
| | | | | system) | | |
| G682 | P26264 | 552 | opLexA | 2-components-supTfn-HA-N-term | N3 | P25976 |
| | | | | (TF component of two-component | | |
| | | | | system) | | |
| G682 | P21166 | 553 | Prom-G682 | Promoter-reporter | N514 | P21142 |
| G682 | P25611 | 554 | Prom-G682 | Promoter-reporter | N514 | P32122 |
| G682 | P25141 | 555 | Prom-G682 | Promoter-reporter (YFP/LTI6b) | N514 | P25755 |
| G682 | P21525 | 556 | SUC2 | Direct promoter-fusion | N1142 | pMEN65 |
| G867 | P21207 | 557 | 35S | Direct GR-fusion C-term | N2 | P21171 |
| G867 | P21282 | 558 | 35S | Direct GR-fusion HA C-term | N2 | P21172 |
| G867 | P21213 | 559 | 35S | Direct GR-fusion N-term | N2 | P21173 |
| G867 | P21288 | 560 | 35S | Direct GR-fusion HA N-term | N2 | P21174 |
| G867 | P21297 | 561 | 35S | RNAi (GS) | N2 | P21103 |
| G867 | P21162 | 562 | 35S | RNAi (clade) | N2 | P21103 |
| G867 | P21303 | 563 | 35S | RNAi (clade) | N2 | P21103 |
| G867 | P21304 | 564 | 35S | RNAi (clade) | N2 | P21103 |
| G867 | P21201 | 565 | 35S | GAL4 N-term | N2 | P21195 |
| G867 | P25301 | 566 | 35S | Protein-GFP-C-fusion | N2 | P25799 |
| G867 | P383 | 567 | 35S | Direct promoter-fusion | N2 | pMEN20 |
| G867 | P21193 | 568 | 35S | GAL4 C-term | N2 | P5425 |
| G867 | P21276 | 569 | 35S | TF dom neg deln 2ndry domain | N2 | pMEN65 |
| G867 | P21275 | 570 | 35S | TF dominant negative deletion | N2 | pMEN65 |
| G867 | P23315 | 571 | ARSK1 | Direct promoter-fusion | N82 | pMEN65 |
| G867 | P7140 | 572 | opLexA | 2-components-supTfn (TF | N3 | P5480 |
| | | | | component of two-component | | |
| | | | | system) | | |
| G867 | P25305 | 573 | opLexA | 2-components-supTfn-TAP-C-term | N3 | P25420 |
| | | | | (TF component of two-component | | |
| | | | | system) | | |
| G867 | P25459 | 574 | opLexA | 2-components-supTfn-HA-C-term | N3 | P25461 |
| | | | | (TF component of two-component | | |
| | | | | system) | | |
| G867 | P26265 | 575 | opLexA | 2-components-supTfn-HA-N-term | N3 | P25976 |
| | | | | (TF component of two-component | | |
| | | | | system) | | |
| G867 | P21170 | 576 | Prom-G867 | Promoter-reporter | N518 | P21142 |
| G867 | P25606 | 577 | Prom-G867 | Promoter-reporter | N518 | P32122 |
| G867 | P21524 | 578 | SUC2 | Direct promoter-fusion | N1142 | pMEN65 |
| G922 | P1898 | 579 | 35S | Direct promoter-fusion | N2 | pMEN65 |
| G922 | P4593 | 580 | opLexA | 2-components-supTfn (TF | N3 | P5381 |
| | | | | component of two-component | | |
| | | | | system) | | |
| G926 | P15491 | 581 | 35S | Direct promoter-fusion | N2 | pMEN65 |
| G926 | P26217 | 582 | 35S | Protein-YFP-C-fusion | N2 | P25800 |
| G927 | P142 | 583 | 35S | Direct Promoter-fusion | N2 | pMEN20 |
| G927 | P26197 | 584 | 35S | Protein-YFP-C-fusion | N2 | P25800 |
| G928 | P143 | 585 | 35S | Direct promoter-fusion | N2 | pMEN20 |
| G928 | P26223 | 586 | 35S | Protein-YFP-C-fusion | N2 | P25800 |
| G993 | P1268 | 587 | 35S | Direct promoter-fusion | N2 | pMEN65 |
| G993 | P21149 | 588 | opLexA | 2-components-supTfn (TF | N3 | P5381 |
| | | | | component of two-component | | |
| | | | | system) | | |
| G1006 | P417 | 589 | 35S | Direct promoter-fusion | N2 | pMEN20 |
| G1006 | P25647 | 590 | Prom-G1006 | Promoter-reporter | N1145 | P21142 |
| G1006 | P25646 | 591 | Prom-G1006 | Promoter-reporter (YFP/LTI6b) | N1145 | P25755 |
| G1667 | P1079 | 592 | 35S | Direct promoter-fusion | N2 | pMEN65 |
| G1067 | P443 | 593 | 35S | Direct promoter-fusion | N2 | pMEN20 |
| G1067 | P7832 | 594 | opLexA | 2-components-supTfn (TF | N3 | P5480 |
| | | | | component of two-component | | |
| | | | | system) | | |
| G1067 | P25099 | 595 | Prom-G1067 | Promoter-reporter | N1095 | P21142 |
| G1069 | P1178 | 596 | 35S | Direct promoter-fusion | N2 | pMEN65 |
| G1069 | P25101 | 597 | Prom-G1069 | Promoter-reporter | N1096 | P21142 |
| G1069 | P25102 | 598 | Prom-G1069 | Promoter-reporter (YFP/LTI6b) | N1096 | P25755 |
| G1073 | P21295 | 599 | 35S | RNAi (GS) | N2 | P21103 |
| G1073 | P21301 | 600 | 35S | RNAi (clade) | N2 | P21103 |
| G1073 | P21205 | 601 | 35S | Direct GR-fusion C-term | N2 | P21171 |
| G1073 | P21280 | 602 | 35S | Direct GR-fusion HA C-term | N2 | P21172 |
| G1073 | P21211 | 603 | 35S | Direct GR-fusion N-term | N2 | P21173 |
| G1073 | P21286 | 604 | 35S | Direct GR-fusion HA N-term | N2 | P21174 |
| G1073 | P21117 | 605 | 35S | RNAi (GS) | N2 | P21103 |
| G1073 | P21160 | 606 | 35S | RNAi (clade) | N2 | P21103 |
| G1073 | P21199 | 607 | 35S | GAL4 N-term | N2 | P21195 |
| G1073 | P25263 | 608 | 35S | Protein-GFP-C-fusion | N2 | P25799 |
| G1073 | P448 | 609 | 35S | Direct promoter-fusion | N2 | pMEN20 |
| G1073 | P21145 | 610 | 35S | GAL4 C-term | N2 | P5425 |
| G1073 | P25703 | 611 | 35S | Direct promoter-fusion | N2 | pMEN65 |
| G1073 | P21271 | 612 | 35S | TF dominant negative deletion | N2 | pMEN65 |
| G1073 | P21272 | 613 | 35S | TF dom neg deln 2ndry domain | N2 | pMEN65 |
| G1073 | P3369 | 614 | opLexA | 2-components-supTfn (TF | N3 | P5480 |
| | | | | component of two-component | | |
| | | | | system) | | |
| G1073 | P25267 | 615 | opLexA | 2-components-supTfn-TAP-C-term | N3 | P25420 |
| | | | | (TF component of two-component | | |
| | | | | system) | | |
| G1073 | P25265 | 616 | opLexA | 2-components-supTfn-HA-C-term | N3 | P25461 |
| | | | | (TF component of two-component | | |
| | | | | system) | | |
| G1073 | P21168 | 617 | Prom-G1073 | Promoter-reporter | N516 | P21142 |
| G1073 | P25104 | 618 | Prom-G1073 | Promoter-reporter (YFP/LTI6b) | N516 | P25755 |
| G1073 | P21521 | 619 | SUC2 | Direct promoter-fusion | N1142 | pMEN65 |
| G1134 | P467 | 620 | 35S | Direct promoter-fusion | N2 | pMEN20 |
| G1248 | P1446 | 621 | 35S | Direct promoter-fusion | N2 | pMEN65 |
| G1248 | P26045 | 622 | 35S | Protein-CFP-C-fusion | N2 | P25801 |
| G1266 | P483 | 623 | 35S | Direct promoter-fusion | N2 | pMEN20 |
| G1266 | P7154 | 624 | opLexA | 2-components-supTfn (TF | N3 | P5480 |
| | | | | component of two-component | | |
| | | | | system) | | |
| G1274 | P25203 | 625 | 35S | Direct GR-fusion C-term | N2 | P21171 |
| G1274 | P25221 | 626 | 35S | Direct GR-fusion N-term | N2 | P21173 |
| G1274 | P25659 | 627 | 35S | GAL4 N-term | N2 | P21195 |
| G1274 | P25658 | 628 | 35S | GAL4 C-term | N2 | P21378 |
| G1274 | P25269 | 629 | 35S | Protein-GFP-C-fusion | N2 | P25799 |
| G1274 | P15038 | 630 | 35S | Direct promoter-fusion | N2 | pMEN1963 |
| G1274 | P25742 | 631 | 35S | site-directed mutation_1 | N2 | pMEN65 |
| G1274 | P25743 | 632 | 35S | site-directed mutation_2 | N2 | pMEN65 |
| G1274 | P25745 | 633 | 35S | site-directed mutation_3 | N2 | pMEN65 |
| G1274 | P25746 | 634 | 35S | site-directed mutation_4 | N2 | pMEN65 |
| G1274 | P25744 | 635 | 35S | site-directed mutation_5 | N2 | pMEN65 |
| G1274 | P25435 | 636 | 35S | domain swap_1 | N2 | pMEN65 |
| G1274 | P25255 | 637 | opLexA | 2-components-supTfn-TAP-C-term | N3 | P25420 |
| | | | | (TF component of two-component | | |
| | | | | system) | | |
| G1274 | P8239 | 638 | opLexA | 2-components-supTfn (TF | N3 | P5480 |
| | | | | component of two-component | | |
| | | | | system) | | |
| G1274 | P25253 | 639 | opLexA | 2-components-supTfn-HA-C-term | N3 | P25461 |
| | | | | (TF component of two-component | | |
| | | | | system) | | |
| G1274 | P26258 | 640 | opLexA | 2-components-supTfn-HA-N-term | N3 | P25976 |
| | | | | (TF component of two-component | | |
| | | | | system) | | |
| G1274 | P25109 | 641 | Prom-G1274 | Promoter-reporter | N1097 | P21142 |
| G1274 | P25110 | 642 | Prom-G1274 | Promoter-reporter (YFP/LTI6b) | N1097 | P25755 |
| G1275 | P486 | 643 | 35S | Direct promoter-fusion | N2 | pMEN20 |
| G1275 | P3412 | 644 | opLexA | 2-components-supTfn (TF | N3 | P5381 |
| | | | | component of two-component | | |
| | | | | system) | | |
| G1275 | P25111 | 645 | Prom-G1275 | Promoter-reporter | N1098 | P21142 |
| G1275 | P25996 | 646 | Prom-G1275 | Promoter-reporter (YFP/LTI6b) | N1098 | P25755 |
| G1334 | P714 | 647 | 35S | Direct promoter-fusion | N2 | pMEN20 |
| G1334 | P26238 | 648 | 35S | Protein-YFP-C-fusion | N2 | P25800 |
| G1364 | P26108 | 649 | 35S | Protein-CFP-C-fusion | N2 | P25801 |
| G1364 | P4357 | 650 | opLexA | 2-components-supTfn (TF | N3 | P5381 |
| | | | | component of two-component | | |
| | | | | system) | | |
| G1752 | P1636 | 651 | 35S | Direct promoter-fusion | N2 | pMEN65 |
| G1752 | P4390 | 652 | opLexA | 2-components-supTfn (TF | N3 | P5381 |
| | | | | component of two-component | | |
| | | | | system) | | |
| G1758 | P1224 | 653 | 35S | Direct promoter-fusion | N2 | pMEN65 |
| G1758 | P25113 | 654 | Prom-G1758 | Promoter-reporter | N1102 | P21142 |
| G1758 | P25114 | 655 | Prom-G1758 | Promoter-reporter (YFP/LTI6b) | N1102 | P25755 |
| G1781 | P965 | 656 | 35S | Direct promoter-fusion | N2 | pMEN65 |
| G1781 | P26043 | 657 | 35S | Protein-CFP-C-fusion | N2 | P25801 |
| G1791 | P25079 | 658 | 35S | Direct GR-fusion C-term | N2 | P21171 |
| G1791 | P25094 | 659 | 35S | Direct GR-fusion HA N-term | N2 | P21173 |
| G1791 | P1694 | 660 | 35S | Direct promoter-fusion | N2 | pMEN65 |
| G1791 | P4406 | 661 | opLexA | 2-components-supTfn (TF | N3 | P5381 |
| | | | | component of two-component | | |
| | | | | system) | | |
| G1791 | P25121 | 662 | Prom-G1791 | Promoter-reporter | N1103 | P21142 |
| G1791 | P25116 | 663 | Prom-G1791 | Promoter-reporter (YFP/LTI6b) | N1103 | P25755 |
| G1792 | P25084 | 664 | 35S | Direct GR-fusion C-term | N2 | P21171 |
| G1792 | P25095 | 665 | 35S | Direct GR-fusion N-term | N2 | P21173 |
| G1792 | P25093 | 666 | 35S | GAL4 N-term | N2 | P21195 |
| G1792 | P25083 | 667 | 35S | GAL4 C-term | N2 | P21378 |
| G1792 | P25438 | 668 | 35S | domain swap_1 | N2 | P21378 |
| G1792 | P25271 | 669 | 35S | Protein-GFP-C-fusion | N2 | P25799 |
| G1792 | P1695 | 670 | 35S | Direct promoter-fusion | N2 | pMEN65 |
| G1792 | P25437 | 671 | 35S | TF dominant negative deletion | N2 | pMEN65 |
| G1792 | P25738 | 672 | 35S | site-directed mutation_1 | N2 | pMEN65 |
| G1792 | P25739 | 673 | 35S | site-directed mutation_2 | N2 | pMEN65 |
| G1792 | P25740 | 674 | 35S | site-directed mutation_3 | N2 | PMEN65 |
| G1792 | P25741 | 675 | 35S | site-directed mutation_4 | N2 | pMEN65 |
| G1792 | P25446 | 676 | 35S | domain swap_2 | N2 | pMEN65 |
| G1792 | P25445 | 677 | 35S | domain swap_5 | N2 | pMEN65 |
| G1792 | P25448 | 678 | 35S | domain swap_4 | N2 | pMEN65 |
| G1792 | P25447 | 679 | 35S | domain swap_3 | N2 | pMEN65 |
| G1792 | P25119 | 680 | opLexA | 2-components-supTfn-TAP-C-term | N3 | P25420 |
| | | | | (TF component of two-component | | |
| | | | | system) | | |
| G1792 | P6071 | 681 | opLexA | 2-components-supTfn (TF | N3 | P5381 |
| | | | | component of two-component | | |
| | | | | system) | | |
| G1792 | P25118 | 682 | opLexA | 2-components-supTfn-HA-C-term | N3 | P25461 |
| | | | | (TF component of two-component | | |
| | | | | system) | | |
| G1792 | P26259 | 683 | opLexA | 2-components-supTfn-HA-N-term | N3 | P25976 |
| | | | | (TF component of two-component | | |
| | | | | system) | | |
| G1792 | P23402 | 684 | Prom-G1792 | Promoter-reporter | N1104 | P21142 |
| G1792 | P25115 | 685 | Prom-G1792 | Promoter-reporter | N1308 | P21142 |
| G1792 | P23306 | 686 | Prom-G1792 | Promoter-reporter | N1104 | P32122 |
| G1792 | P25942 | 687 | Prom-G1792 | Promoter-reporter | N1170 | P21142 |
| G1792 | P25943 | 688 | Prom-G1792 | Promoter-reporter(YFP/LTI6b) | N1170 | P25755 |
| G1795 | P1575 | 689 | 35S | Direct promoter-fusion | N2 | pMEN65 |
| G1795 | P25085 | 690 | 35S | Direct GR-fusion C-term | N2 | P21171 |
| G1795 | P25096 | 691 | 35S | Direct GR-fusion HA N-term | N2 | P21173 |
| G1795 | P6424 | 692 | opLexA | 2-components-supTfn (TF | N3 | P5480 |
| | | | | component of two-component | | |
| | | | | system) | | |
| G1816 | P8223 | 693 | opLexA | 2-components-supTfn (TF | N3 | P5480 |
| | | | | component of two-component | | |
| | | | | system) | | |
| G1818 | P1677 | 694 | 35S | Direct promoter-fusion | N2 | pMEN65 |
| G1818 | P26159 | 695 | 35S | Protein-YFP-C-fusion | N2 | P25800 |
| G1819 | P1285 | 696 | 35S | Direct promoter-fusion | N2 | pMEN65 |
| G1819 | P26065 | 697 | 35S | Protein-YFP-C-fusion | N2 | P25800 |
| G1820 | P1284 | 698 | 35S | Direct promoter-fusion | N2 | pMEN65 |
| G1820 | P26064 | 699 | 35S | Protein-YFP-C-fusion | N2 | P25800 |
| G1820 | P3372 | 700 | opLexA | 2-components-supTfn (TF | N3 | P5480 |
| | | | | component of two-component | | |
| | | | | system) | | |
| G1821 | P26037 | 701 | 35S | Protein-CFP-C-fusion | N2 | P25801 |
| G1836 | P26052 | 702 | 35S | Protein-YFP-C-fusion | N2 | P25800 |
| G1836 | P3603 | 703 | opLexA | 2-components-supTfn (TF | N3 | P5381 |
| | | | | component of two-component | | |
| | | | | system) | | |
| G1919 | P1581 | 704 | 35S | Direct promoter-fusion | N2 | pMEN65 |
| G1927 | P2029 | 705 | 35S | Direct promoter-fusion | N2 | pMEN65 |
| G1930 | P1310 | 706 | 35S | Direct promoter-fusion | N2 | pMEN65 |
| G1930 | P3373 | 707 | opLexA | 2-components-supTfn (TF | N3 | P5480 |
| | | | | component of two-component | | |
| | | | | system) | | |
| G2010 | P1278 | 708 | 35S | Direct promoter-fusion | N2 | pMEN65 |
| G2053 | P2032 | 709 | 35S | Direct promoter-fusion | N2 | pMEN65 |
| G2115 | P1507 | 710 | 35S | Direct promoter-fusion | N2 | pMEN65 |
| G2133 | P1572 | 711 | 35S | Direct promoter-fusion | N2 | pMEN65 |
| G2133 | P4361 | 712 | opLexA | 2-components-supTfn (TF | N3 | P5381 |
| | | | | component of two-component | | |
| | | | | system) | | |
| G2133 | P25132 | 713 | Prom-G2133 | Promoter-reporter | N1108 | P21142 |
| G2133 | P25133 | 714 | Prom-G2133 | Promoter-reporter (YFP/LTI6b) | N1108 | P25755 |
| G2149 | P2065 | 715 | 35S | Direct promoter-fusion | N2 | pMEN1963 |
| G2153 | P1740 | 716 | 35S | Direct promoter-fusion | N2 | pMEN65 |
| G2153 | P4524 | 717 | opLexA | 2-components-supTfn (TF | N3 | P5381 |
| | | | | component of two-component | | |
| | | | | system) | | |
| G2153 | P25105 | 718 | Prom-G2153 | Promoter-reporter | N1110 | P21142 |
| G2156 | P1721 | 719 | 35S | Direct promoter-fusion | N2 | pMEN65 |
| G2156 | P4418 | 720 | opLexA | 2-components-supTfn (TF | N3 | P5381 |
| | | | | component of two-component | | |
| | | | | system) | | |
| G2156 | P25107 | 721 | Prom-G2156 | Promoter-reporter | N1111 | P21142 |
| G2157 | P1722 | 722 | 35S | Direct promoter-fusion | N2 | pMEN65 |
| G2345 | P26296 | 723 | 35S | Protein-CFP-C-fusion | N2 | P25801 |
| G2345 | P8079 | 724 | opLexA | 2-components-supTfn (TF | N3 | P5480 |
| | | | | component of two-component | | |
| | | | | system) | | |
| G2517 | P1833 | 725 | 35S | Direct promoter-fusion | N2 | pMEN65 |
| G2539 | P13710 | 726 | 35S | Direct promoter-fusion | N2 | pMEN1963 |
| G2555 | P2069 | 727 | 35S | Direct promoter-fusion | N2 | pMEN65 |
| G2637 | P13696 | 728 | 35S | Direct promoter-fusion | N2 | pMEN1963 |
| G2637 | P26054 | 729 | 35S | Protein-YFP-C-fusion | N2 | P25800 |
| G2718 | P8664 | 730 | opLexA | 2-components-supTfn (TF | N3 | P5480 |
| | | | | component of two-component | | |
| | | | | system) | | |
| G2718 | P23528 | 731 | Prom-G2718 | Promoter-reporter | N1116 | P21142 |
| G2718 | P25139 | 732 | Prom-G2718 | Promoter-reporter (YFP/LTI6b) | N1116 | P25755 |
| G2766 | P2532 | 733 | 35S | Direct promoter-fusion | N2 | pMEN1963 |
| G2989 | P2425 | 734 | 35S | Direct promoter-fusion | N2 | pMEN1963 |
| G2990 | P2426 | 735 | 35S | Direct promoter-fusion | N2 | pMEN1963 |
| G2991 | P2423 | 736 | 35S | Direct promoter-fusion | N2 | pMEN1963 |
| G2992 | P2427 | 737 | 35S | Direct promoter-fusion | N2 | pMEN1963 |
| G2993 | P13792 | 738 | 35S | Direct promoter-fusion | N2 | pMEN1963 |
| G2994 | P2434 | 739 | 35S | Direct promoter-fusion | N2 | pMEN1963 |
| G2995 | P25364 | 740 | 35S | Direct promoter-fusion | N2 | pMEN65 |
| G2996 | P2424 | 741 | 35S | Direct promoter-fusion | N2 | pMEN1963 |
| G2997 | P15364 | 742 | 35S | Direct promoter-fusion | N2 | pMEN65 |
| G2998 | P2431 | 743 | 35S | Direct promoter-fusion | N2 | pMEN1963 |
| G2999 | P25148 | 744 | 35S | Direct GR-fusion C-term | N2 | P21171 |
| G2999 | P25174 | 745 | 35S | Direct GR-fusion N-term | N2 | P21173 |
| G2999 | P25173 | 746 | 35S | GAL4 N-term | N2 | P21195 |
| G2999 | P25147 | 747 | 35S | GAL4 C-term | N2 | P21378 |
| G2999 | P25275 | 748 | 35S | Protein-GFP-C-fusion | N2 | P25799 |
| G2999 | P15277 | 749 | 35S | Direct promoter-fusion | N2 | pMEN1963 |
| G2999 | P25737 | 750 | 35S | site-directed mutation_1 | N2 | pMEN65 |
| G2999 | P25736 | 751 | 35S | site-directed mutation_2 | N2 | pMEN65 |
| G2999 | P25191 | 752 | opLexA | 2-components-supTfn-TAP-C-term | N3 | P25420 |
| | | | | (TF component of two-component | | |
| | | | | system) | | |
| G2999 | P8587 | 753 | opLexA | 2-components-supTfn (TF | N3 | P5480 |
| | | | | component of two-component | | |
| | | | | system) | | |
| G2999 | P25190 | 754 | opLexA | 2-components-supTfn-HA-C-term | N3 | P25461 |
| | | | | (TF component of two-component | | |
| | | | | system) | | |
| G2999 | P26260 | 755 | opLexA | 2-components-supTfn-HA-N-term | N3 | P25976 |
| | | | | (TF component of two-component | | |
| | | | | system) | | |
| G3000 | P23554 | 756 | 35S | Direct promoter-fusion | N2 | pMEN65 |
| G3001 | P2433 | 757 | 35S | Direct promoter-fusion | N2 | pMEN1963 |
| G3002 | P15113 | 758 | 35S | Direct promoter-fusion | N2 | pMEN1963 |
| G3074 | P2712 | 759 | 35S | Direct promoter-fusion | N2 | pMEN1963 |
| G3074 | P26055 | 760 | 35S | Protein-YFP-C-fusion | N2 | P25800 |
| G3086 | P25664 | 761 | 35S | Direct GR-fusion N-term | N2 | P21173 |
| G3086 | P25662 | 762 | 35S | GAL4 N-term | N2 | P21195 |
| G3086 | P25660 | 763 | 35S | GAL4 C-term | N2 | P21378 |
| G3086 | P25277 | 764 | 35S | Protein-GFP-C-fusion | N2 | P25799 |
| G3086 | P15046 | 765 | 35S | Direct promoter-fusion | N2 | pMEN1963 |
| G3086 | P26196 | 766 | 35S | Direct GR-fusion C-term | N2 | P21171 |
| G3086 | P8242 | 767 | opLexA | 2-components-supTfn (TF | N3 | P5480 |
| | | | | component of two-component | | |
| | | | | system) | | |
| G3086 | P25756 | 768 | opLexA | 2-components-supTfn-TAP-C-term | N3 | P25420 |
| | | | | (TF component of two-component | | |
| | | | | system) | | |
| G3086 | P25757 | 769 | opLexA | 2-components-supTfn-HA-C-term | N3 | P25461 |
| | | | | (TF component of two-component | | |
| | | | | system) | | |
| G3086 | P25128 | 770 | Prom-G3086 | Promoter-reporter | N1119 | P21142 |
| G3086 | P25129 | 771 | Prom-G3086 | Promoter-reporter (YFP/LTI6b) | N1119 | P25755 |
| G3380 | P21460 | 772 | 35S | Direct promoter-fusion | N2 | pMEN65 |
| G3381 | P21461 | 773 | 35S | Direct promoter-fusion | N2 | pMEN65 |
| G3381 | P25098 | 774 | opLexA | 2-components-supTfn (TF | N3 | pMEN65 |
| | | | | component of two-component | | |
| | | | | system) | | |
| G3383 | P23523 | 775 | 35S | Direct promoter-fusion | N2 | pMEN65 |
| G3388 | P21266 | 776 | 35S | Direct promoter-fusion | N2 | pMEN65 |
| G3388 | P21327 | 777 | 35S | Direct promoter-fusion | N2 | pMEN65 |
| G3389 | P21260 | 778 | 35S | Direct promoter-fusion | N2 | pMEN65 |
| G3390 | P21375 | 779 | 35S | Direct promoter-fusion | N2 | pMEN65 |
| G3390 | P21258 | 780 | 35S | Direct promoter-fusion | N2 | pMEN65 |
| G3391 | P21257 | 781 | 35S | Direct promoter-fusion | N2 | pMEN65 |
| G3392 | P21255 | 782 | 35S | Direct promoter-fusion | N2 | pMEN65 |
| G3393 | P21254 | 783 | 35S | Direct promoter-fusion | N2 | pMEN65 |
| G3393 | P21256 | 784 | 35S | Direct promoter-fusion | N2 | pMEN65 |
| G3394 | P21248 | 785 | 35S | Direct promoter-fusion | N2 | pMEN65 |
| G3394 | P23384 | 786 | 35S | Direct promoter-fusion | N2 | pMEN65 |
| G3394 | P23481 | 787 | 35S | Direct promoter-fusion | N2 | pMEN65 |
| G3395 | P21253 | 788 | 35S | Direct promoter-fusion | N2 | pMEN65 |
| G3396 | P23304 | 789 | 35S | Direct promoter-fusion | N2 | pMEN65 |
| G3397 | P21265 | 790 | 35S | Direct promoter-fusion | N2 | pMEN65 |
| G3398 | P21252 | 791 | 35S | Direct promoter-fusion | N2 | pMEN65 |
| G3399 | P21269 | 792 | 35S | Direct promoter-fusion | N2 | pMEN65 |
| G3399 | P21465 | 793 | 35S | Direct promoter-fusion | N2 | pMEN65 |
| G3400 | P21244 | 794 | 35S | Direct promoter-fusion | N2 | pMEN65 |
| G3401 | P21264 | 795 | 35S | Direct promoter-fusion | N2 | pMEN65 |
| G3406 | P21238 | 796 | 35S | Direct promoter-fusion | N2 | pMEN65 |
| G3407 | P21243 | 797 | 35S | Direct promoter-fusion | N2 | pMEN65 |
| G3408 | P21246 | 798 | 35S | Direct promoter-fusion | N2 | pMEN65 |
| G3429 | P21251 | 799 | 35S | Direct promoter-fusion | N2 | pMEN65 |
| G3430 | P21267 | 800 | 35S | Direct promoter-fusion | N2 | pMEN65 |
| G3431 | P21324 | 801 | 35S | Direct promoter-fusion | N2 | pMEN65 |
| G3432 | P21318 | 802 | 35S | Direct promoter-fusion | N2 | pMEN65 |
| G3434 | P21466 | 803 | 35S | Direct promoter-fusion | N2 | pMEN65 |
| G3435 | P21314 | 804 | 35S | Direct promoter-fusion | N2 | pMEN65 |
| G3436 | P21381 | 805 | 35S | Direct promoter-fusion | N2 | pMEN65 |
| G3436 | P21315 | 806 | 35S | Direct promoter-fusion | N2 | pMEN65 |
| G3444 | P21320 | 807 | 35S | Direct promoter-fusion | N2 | pMEN65 |
| G3445 | P21352 | 808 | 35S | Direct promoter-fusion | N2 | pMEN65 |
| G3446 | P21353 | 809 | 35S | Direct promoter-fusion | N2 | pMEN65 |
| G3447 | P21354 | 810 | 35S | Direct promoter-fusion | N2 | pMEN65 |
| G3448 | P21355 | 811 | 35S | Direct promoter-fusion | N2 | pMEN65 |
| G3449 | P21356 | 812 | 35S | Direct promoter-fusion | N2 | pMEN65 |
| G3450 | P21351 | 813 | 35S | Direct promoter-fusion | N2 | pMEN65 |
| G3451 | P21500 | 814 | 35S | Direct promoter-fusion | N2 | pMEN65 |
| G3452 | P21501 | 815 | 35S | Direct promoter-fusion | N2 | pMEN65 |
| G3453 | P23348 | 816 | 35S | Direct promoter-fusion | N2 | pMEN65 |
| G3455 | P21495 | 817 | 35S | Direct promoter-fusion | N2 | pMEN65 |
| G3456 | P21328 | 818 | 35S | Direct promoter-fusion | N2 | pMEN65 |
| G3456 | P21467 | 819 | 35S | Direct promoter-fusion | N2 | pMEN65 |
| G3458 | P21330 | 820 | 35S | Direct promoter-fusion | N2 | pMEN65 |
| G3459 | P21331 | 821 | 35S | Direct promoter-fusion | N2 | pMEN65 |
| G3460 | P21332 | 822 | 35S | Direct promoter-fusion | N2 | pMEN65 |
| G3470 | P21341 | 823 | 35S | Direct promoter-fusion | N2 | pMEN65 |
| G3470 | P21471 | 824 | 35S | Direct promoter-fusion | N2 | pMEN65 |
| G3471 | P21342 | 825 | 35S | Direct promoter-fusion | N2 | pMEN65 |
| G3472 | P21348 | 826 | 35S | Direct promoter-fusion | N2 | pMEN65 |
| G3474 | P21344 | 827 | 35S | Direct promoter-fusion | N2 | pMEN65 |
| G3474 | P21469 | 828 | 35S | Direct promoter-fusion | N2 | pMEN65 |
| G3475 | P21347 | 829 | 35S | Direct promoter-fusion | N2 | pMEN65 |
| G3476 | P21345 | 830 | 35S | Direct promoter-fusion | N2 | pMEN65 |
| G3478 | P21350 | 831 | 35S | Direct promoter-fusion | N2 | pMEN65 |
| G3515 | P21401 | 832 | 35S | Direct promoter-fusion | N2 | pMEN65 |
| G3516 | P21402 | 833 | 35S | Direct promoter-fusion | N2 | pMEN65 |
| G3517 | P21403 | 834 | 35S | Direct promoter-fusion | N2 | pMEN65 |
| G3518 | P21404 | 835 | 35S | Direct promoter-fusion | N2 | pMEN65 |
| G3519 | P21405 | 836 | 35S | Direct promoter-fusion | N2 | pMEN65 |
| G3520 | P21406 | 837 | 35S | Direct promoter-fusion | N2 | pMEN65 |
| G3556 | P21493 | 838 | 35S | Direct promoter-fusion | N2 | pMEN65 |
| G3643 | P23465 | 839 | 35S | Direct promoter-fusion | N2 | pMEN65 |
| G3644 | P23455 | 840 | 35S | Direct promoter-fusion | N2 | pMEN65 |
| G3644 | P25188 | 841 | opLexA | 2-components-supTfn (TF | N3 | P5381 |
| | | | | component of two-component | | |
| | | | | system) | | |
| G3649 | P23456 | 842 | 35S | Direct promoter-fusion | N2 | pMEN65 |
| G3650 | P25402 | 843 | 35S | Direct promoter-fusion | N2 | pMEN65 |
| G3659 | P23452 | 844 | 35S | Direct promoter-fusion | N2 | pMEN65 |
| G3660 | P23418 | 845 | 35S | Direct promoter-fusion | N2 | pMEN65 |
| G3661 | P23419 | 846 | 35S | Direct promoter-fusion | N2 | pMEN65 |
| G3676 | P25159 | 847 | 35S | Direct promoter-fusion | N2 | pMEN65 |
| G3681 | P25163 | 848 | 35S | Direct promoter-fusion | N2 | pMEN65 |
| G3685 | P25166 | 849 | 35S | Direct promoter-fusion | N2 | pMEN65 |
| G3686 | P25167 | 850 | 35S | Direct promoter-fusion | N2 | pMEN65 |
| G3690 | P25407 | 851 | 35S | Direct promoter-fusion | N2 | pMEN65 |
| G3717 | P23421 | 852 | 35S | Direct promoter-fusion | N2 | pMEN65 |
| G3718 | P23423 | 853 | 35S | Direct promoter-fusion | N2 | pMEN65 |
| G3719 | P25204 | 854 | 35S | Direct promoter-fusion | N2 | pMEN65 |
| G3720 | P25205 | 855 | 35S | Direct promoter-fusion | N2 | pMEN65 |
| G3721 | P25368 | 856 | 35S | Direct promoter-fusion | N2 | pMEN65 |
| G3722 | P25207 | 857 | 35S | Direct promoter-fusion | N2 | pMEN65 |
| G3723 | P25208 | 858 | 35S | Direct promoter-fusion | N2 | pMEN65 |
| G3724 | P25384 | 859 | 35S | Direct promoter-fusion | N2 | pMEN65 |
| G3724 | P25222 | 860 | opLexA | 2-components-supTfn (TF | N3 | pMEN53 |
| | | | | component of two-component | | |
| | | | | system) | | |
| G3725 | P25210 | 861 | 35S | Direct promoter-fusion | N2 | pMEN65 |
| G3726 | P25211 | 862 | 35S | Direct promoter-fusion | N2 | pMEN65 |
| G3727 | P25385 | 863 | 35S | Direct promoter-fusion | N2 | pMEN65 |
| G3728 | P25213 | 864 | 35S | Direct promoter-fusion | N2 | pMEN65 |
| G3729 | P25214 | 865 | 35S | Direct promoter-fusion | N2 | pMEN65 |
| G3730 | P25215 | 866 | 35S | Direct promoter-fusion | N2 | pMEN65 |
| G3737 | P25089 | 867 | 35S | Direct promoter-fusion | N2 | pMEN65 |
| G3739 | P25090 | 868 | 35S | Direct promoter-fusion | N2 | pMEN65 |
| G3742 | P25661 | 869 | 35S | Direct promoter-fusion | N2 | pMEN65 |
| G3744 | P25370 | 870 | 35S | Direct promoter-fusion | N2 | pMEN65 |
| G3746 | P25230 | 871 | 35S | Direct promoter-fusion | N2 | pMEN65 |
| G3750 | P25233 | 872 | 35S | Direct promoter-fusion | N2 | pMEN65 |
| G3755 | P25426 | 873 | 35S | Direct promoter-fusion | N2 | pMEN65 |
| G3760 | P25360 | 874 | 35S | Direct promoter-fusion | N2 | pMEN65 |
| G3765 | P25241 | 875 | 35S | Direct promoter-fusion | N2 | pMEN65 |
| G3766 | P25242 | 876 | 35S | Direct promoter-fusion | N2 | pMEN65 |
| G3767 | P25243 | 877 | 35S | Direct promoter-fusion | N2 | pMEN65 |
| G3768 | P25244 | 878 | 35S | Direct promoter-fusion | N2 | pMEN65 |
| G3769 | P25245 | 879 | 35S | Direct promoter-fusion | N2 | pMEN65 |
| G3771 | P25246 | 880 | 35S | Direct promoter-fusion | N2 | pMEN65 |
| G3794 | P25092 | 881 | 35S | Direct promoter-fusion | N2 | pMEN65 |
| G3803 | P25218 | 882 | 35S | Direct promoter-fusion | N2 | pMEN65 |
| G3804 | P25219 | 883 | 35S | Direct promoter-fusion | N2 | pMEN65 |
| G3841 | P25573 | 884 | 35S | Direct promoter-fusion | N2 | pMEN65 |
| G3848 | P25571 | 885 | 35S | Direct promoter-fusion | N2 | pMEN65 |
| G3856 | P25572 | 886 | 35S | Direct promoter-fusion | N2 | pMEN65 |
| G3864 | P25578 | 887 | 35S | Direct promoter-fusion | N2 | pMEN65 |
| G3876 | P25657 | 888 | 35S | Direct promoter-fusion | N2 | pMEN65 |
| n/a | P6506 | 889 | 35S | Promoter background | N2 | P5386 |
| | | | | (Promoter::LexA-GAL4TA driver | | |
| | | | | construct in 2-component system) | | |
| n/a | P5486 | 890 | 35SLEXA::GR | Promoter background | N2 | pMEN57 |
| | | | | (Promoter::LexA-GAL4TA driver | | |
| | | | | construct in 2-component system) | | |
| n/a | P5326 | 891 | AP1 | Promoter background | N207 | P5375 |
| | | | | (Promoter::LexA-GAL4TA driver | | |
| | | | | construct in 2-component system) | | |
| n/a | P5311 | 892 | ARSK1 | Promoter background | N82 | P5375 |
| | | | | (Promoter::LexA-GAL4TA driver | | |
| | | | | construct in 2-component system) | | |
| n/a | P5319 | 893 | AS1 | Promoter background | N179 | P5375 |
| | | | | (Promoter::LexA-GAL4TA driver | | |
| | | | | construct in 2-component system) | | |
| n/a | P5288 | 894 | CUT1 | Promoter background | N19 | P5375 |
| | | | | (Promoter::LexA-GAL4TA driver | | |
| | | | | construct in 2-component system) | | |
| n/a | P5287 | 895 | LTP1 | Promoter background | N18 | P5375 |
| | | | | (Promoter::LexA-GAL4TA driver | | |
| | | | | construct in 2-component system) | | |
| n/a | P5284 | 896 | RBCS3 | Promoter background | N11 | P5375 |
| | | | | (Promoter::LexA-GAL4TA driver | | |
| | | | | construct in 2-component system) | | |
| n/a | P9002 | 897 | RD29A | Promoter background | N249 | P5375 |
| | | | | (Promoter::LexA-GAL4TA driver | | |
| | | | | construct in 2-component system) | | |
| n/a | P5310 | 898 | RSI1 | Promoter background | N81 | P5375 |
| | | | | (Promoter::LexA-GAL4TA driver | | |
| | | | | construct in 2-component system) | | |
| n/a | P5318 | 899 | STM | Promoter background | N178 | P5375 |
| | | | | (Promoter::LexA-GAL4TA driver | | |
| | | | | construct in 2-component system) | | |
| n/a | P5290 | 900 | SUC2 | Promoter background | N23 | P5375 |
| | | | | (Promoter::LexA-GAL4TA driver | | |
| | | | | construct in 2-component system) | | |

The Two-Component Expression System

For the two-component system, two separate constructs are used: Promoter::LexA-GAL4TA and opLexA::TF. The first of these (Promoter::LexA-GAL4TA) comprises a desired promoter cloned in front of a LexA DNA binding domain fused to a GAL4 activation domain. The construct vector backbone (pMEN48, also known as P5375, SEQ ID NO: 906) also carries a kanamycin resistance marker, along with an opLexA::GFP reporter. Transgenic lines are obtained containing this first component, and a line is selected that shows reproducible expression of the reporter gene in the desired pattern through a number of generations. A homozygous population is established for that line, and the population is supertransformed with the second construct (opLexA::TF) carrying the TF of interest cloned behind a LexA operator site. This second construct vector backbone (pMEN53, also known as P5381, SEQ ID NO: 908) also contains a sulfonamide resistance marker.

Each of the above methods offers a number of pros and cons. A direct fusion approach allows for much simpler genetic analysis if a given promoter-TF line is to be crossed into different genetic backgrounds at a later date. The two-component method, on the other hand, potentially allows for stronger expression to be obtained via an amplification of transcription. Additionally, a range of two-component constructs were available at the start of the Lead Advancement program which had been built using funding from an Advanced Technology Program (ATP) grant.

In general, the lead TF from each study group is expressed from a range of different promoters using a two component method. Arabidopsis paralogs are also generally analyzed by the two-component method, but are typically analyzed using the only 35S promoter. However, an alternative promoter is sometimes used for paralogs when there is already a specific indication that a different promoter might afford a more useful approach (such as when use of the 35S promoter is already known to generate deleterious effects). Putative orthologs from other species are usually analyzed by overexpression from a 35S CaMV promoter via a direct promoter-fusion construct. The vector backbone for most of the direct promoter-fusion overexpression constructs is pMEN65, but pMEN1963 and pMEN20 are sometimes used.

### (2) Knock-Out/Knock-Down

Where available, T-DNA insertion lines from either the public or the in-house collections are analyzed.

In cases where a T-DNA insertion line is unavailable, an RNA interference (RNAi) strategy is sometimes used. At the outset of the program, the system was tested with two well-characterized genes [LEAFY (Weigel et al., 1992) and CONSTANS (Putterill et al., 1995)] that give clear morphological phenotypes when mutated. In each case, RNAi lines were obtained that exhibited characters seen in the null mutants.

An RNAi based strategy was taken for each of the five initial drought leads (Module 1). The approaches and target fragments that were planned for several Arabidopsis transcription factor sequences are shown in Table 19 and Table 20. For each lead gene, two constructs were designed: one being targeted to the lead gene itself and the other being targeted to the conserved domain shared by all the Arabidopsis paralogs. In some cases the RNAi fragments that were originally planned differ slightly from those that were finally included in the constructs. In such cases those differences, along with the DNA sequence of the full insert within the RNAi construct, are provided in the sequence section of the RNAi project reports for that gene. For two of the genes, G481 and G867, two alternative constructs targeting the clade of related genes were generated. Details of those constructs, G481-RNAi (clade) (P21159, P21300, P21305), and G867-RNAi (clade) (P21303, P21162, P21304), are provided in the Sequence Listing.

TABLE 19
Summary of fragments contained within gene specific
RNAi constructs for five primary genes

| GID | Target Region from ATG | Element Size |
|---|---|---|
| G682 | 191-342 | 151 bps |
| G481 | 277-677 | 400 bps |
| G1073 | 208-711 | 503 bps |
| G867 | 869-1198 | 330 bps |

Note:
The vector for all RNAi constructs (P21103) is derived from pMEN65 (Example II). A PDK intron (Waterhouse et al., 2001) was cloned into the middle of the multiple cloning sites in pMEN65, to produce this vector.

TABLE 20

Summary of fragments contained within Clade-Targeted RNAi Constructs. The entry vector for all RNAi
constructs is derived from pMEN65. A PDK intron (Waterhouse et al. (2001) was cloned into the middle
of the multiple cloning sites in pMEN65, which resulted in the entry vector.

G682
Two fragments, one from G682 and the other from G1816, will be generated and ligated together to
generate a hybrid fragment targeting the G682 clade members.
Fragment 1 sequence (125 bp) based on G682 CDS:
cttcttgttccgaagaggtgagtagtcttgagtgggaagttgtgaacatgagtcaagaagaagaagatttggtctctcgaatgcataagcttgtcggtgacag
gtgggagttgatcgccggaagg
Fragment 2 sequence (162 bp) based on G1816 CDS:
gaagtgagt ag c
atcgaatgggagtttatcaacatgactgaacaagaagaagatctcatctttcgaatgtacagacttgtcggtgataggtgggatttgatagcaggaagagttc
ctggaagacaaccagaggagatagagagata c tggat t atgagaaac

The bold italicized bases indicate positions where point mutations were introduced in the cloning
primers to increase the percentage homology with other clade members. The percentage homology of the
above fragment to each target clade member is shown below.

| | |
|---|---|
| Fragment 1 | Fragment 2 |

| | | | |
|---|---|---|---|
| GID | Homology (%) | GID | Homology (%) |

| | | | |
|---|---|---|---|
| G682 | 117/125 (93%) | G1816 | 158/162 (97%) |
| G225 | 106/125 (85%) | G226 | 148/162 (91%) |

G481
Two fragments, one from G485 and the other from G2345, will be generated and ligated together to
generate a hybrid fragment targeting G482 clade members.
Fragment 1 sequence (110 bp) based on G485 CDS:
gagcaagacaggttcttaccgatcgctaacgttagcaggatcatgaagaaagcacttcctgcgaacgcaaaaatctctaaggatgctaaagaaacgatgcagg
agtgtgt
Fragment 2 sequence (131 bp) based on G2345 CDS:
aggaatgcgtctctgagttcatcagcttcgtcaccagcgaggctagtgataagtgccaaagagagaaaaggaagaccatcaatggagatgatttgctttgggc
tatggccactttaggatttgaggattac

The bold italicized bases indicate positions where point mutations were introduced in the cloning
primers to increase the percentage homology with other clade members. The percentage homology of the
above fragment to each target clade member is shown below.

| | |
|---|---|
| Fragment 1 | Fragment 2 |

| | | | |
|---|---|---|---|
| GID | Homology (%) | GID | Homology (%) |

| | | | |
|---|---|---|---|
| G482 | 96/110 (87%) | G481 | 116/131 (88%) |
| G485 | 104/110 (94%) | G1364 | 118/131 (90%) |
| | | G2345 | 127/131 (97%) |
| | | G482 | 110/131 (84%) |

G1073
A 102 bp fragment will be generated based on the G2156 CDS between positions 216 and 318 counting from
first base of the start codon.
cgtccacgtggtcgtcctgcgggatccaagaacaagccgaagccaccggtgatagtgactagagatagccccaacgtgcttagatcacacgttcttgaagtc

The bold italicized bases indicate positions where point mutations were introduced in the cloning
primers to increase the percentage homology with other clade members. The percentage homology of the
above fragment to each target clade member is shown below.

| | |
|---|---|
| GID | Homology (%) |

| | |
|---|---|
| G1073 | 87/102 (85%) |
| G1067 | 86/102 (84%) |
| G2156 | 98/102 (96%) |

G867
A 127 bp fragment will be generated based on the G867 CDS between positions 163 and 290 counting from
the first base of the start codon.
gaaagcttccgtcgtcaaaatacaaaggtgtggtgccacaaccaaacggaagatggggagctcagatttacgagaaacaccagcgcgtgtggctcgggacatt
caacgaggaagaagaagccgctcg

The bold italicized bases indicate positions where point mutations were introduced in the cloning
primers to increase the percentage homology with other clade members. The percentage homology of the
above fragment to each target clade member is shown below.

| | |
|---|---|
| GID | Homology (%) |

| | |
|---|---|
| G867 | 123/125 (98%) |
| G9 | 111/127 (87%) |
| G993 | 105/119 (88%) |
| G1930 | 112/127 (88%) |

### (3) Protein Modifications

Addition of Non-Native Activation Domains

Translational fusions to a GAL4 acidic activation domain may be used in an attempt to alter TF potency.

Other activation domains such as VP16 may also be considered in the future.

### Deletion Variants

Truncated versions or fragments of the leads are sometimes overexpressed to test hypotheses regarding particular parts of the proteins. Such an approach can result in dominant negative alleles.

Point Mutation and Domain Swap Variants

In order to assess the role of particular conserved residues or domains, mutated versions of lead proteins with substitutions at those residues are overexpressed. In some cases, we also overexpress chimeric variants of the transcription factor in which one or domains have been exchanged with another transcription factor.

### (4) Analytical Tools for Pathway Analysis

Promoter-Reporter Constructs

Promoters are primarily cloned in front of a GUS reporter system. These constructs can be used to identify putative upstream transcriptional activators via a transient assay. In most cases approximately 2 kb of the sequence immediately 5′ to the ATG of the gene was included in the construct. The exact promoter sequences included in these constructs are provided in the Sequence Listing.

In addition to being used in transient assays, the promoter-reporter constructs are transformed into Arabidopsis. The lines are then used to characterize the expression patterns of the lead genes in planta over a variety of tissue types and stress conditions. As well as GUS, a number of fluorescent reporter proteins are used in Promoter-reporter constructs including GFP, YFP, CFP and anchored variants of YFP such as YFP-LTI6.

Protein Fusions to Fluorescent Tags

To examine sub-cellular localization of TFs, translational fusions to fluorescent markers such as GFP, CFP, and YFP are used.

Dexamethasone Inducible Lines

Glucocorticoid receptor fusions at the N and C termini of the primary TFs are being constructed to allow the identification of their immediate/early targets during array-based studies. We also produce dexamethasone inducible lines via a two-component approach.

Epitope-Tagged Variants

A number of epitope-tagged variants of each lead TF are being generated. Transgenic lines for these variants are for use in chromatin immunoprecipitation experiments (ChIP) and mass spectrometry based studies to assess protein-protein interactions and the presence of post-translational modifications. For each lead, the following are typically being made: TF-HA, HA-TF, and TF-TAP (HA=hemagglutinin epitope tag, TAP=a tandem affinity purification tag).
Definitions of particular project types, as referenced in the phenotypic screen report sections are provided in Table 21.

TABLE 21

| Project type | Definition |
|---|---|
| Direct promoter-fusion | A full-length wild-type version of a gene is directly fused to a promoter that will drive |
| (DPF) | its expression in transgenic plants. Such a promoter could be the native promoter or that |
| | gene, 35S, or a promoter that will drive tissue specific or conditional expression. |
| 2-components-supTfn | A full-length wild-type version of a gene is being expressed via the 2 component, |
| (TCST) | promoter::LexA-GAL4; opLexA::TF system. In this case, a stable transgenic line is first |
| | established containing one of the components and is later supertransformed with the |
| | second component. |
| splice_variant_* | A splice variant of a gene is directly fused to a promoter that will drive its expression in |
| | transgenic plants. Such a promoter could be the native promoter or that gene, 35S, or a |
| | promoter that will drive tissue specific or conditional expression. |
| Direct GR-fusion C- | A construct contains a TF with a direct C-terminal fusion to a glucocorticoid receptor. |
| term | |
| Direct GR-fusion N- | A construct contains a TF with a direct N-terminal fusion to a glucocorticoid receptor. |
| term | |
| Direct GR-fusion HA | A construct contains a TF with a direct C-terminal fusion to a glucocorticoid receptor in |
| C-term | combination with an HA (hemagglutinin) epitope tag in the conformation: TF-GR-HA |
| Direct GR-fusion HA | A construct contains a TF with a direct N-terminal fusion to a glucocorticoid receptor in |
| N-term | combination with an HA (hemagglutinin) epitope tag in the conformation: GR-TF-HA |
| GAL4 C-term | A TF with a C-terminal fusion to a GAL4 activation domain is being overexpressed. |
| GAL4 N-term | A TF with an N-terminal fusion to a GAL4 activation domain is being overexpressed. |
| TF dominant negative | A truncated variant or fragment of a TF is being (over)expressed, often with the aim of |
| deletion | producing a dominant negative phenotype. Usually the truncated version comprises the |
| | DNA binding domain. Projects of this category are presented in the results tables of our |
| | reports under the sections on “deletion variants. |
| TF dom neg deln 2ndry | A truncated variant or fragment of a TF is being (over)expressed, often with the aim of |
| domain | producing a dominant negative phenotype. In this case, the truncated version contains a |
| | conserved secondary domain (rather than the main DNA binding domain) or a |
| | secondary DNA binding domain alone, in the case when a TF has two potential binding |
| | domain (e.g. B3 & AP2). Projects of this category are presented in the results tables of |
| | our reports under the sections on “deletion variants. |
| deletion_* | A variant of a TF is being (over)expressed in which one or more regions have been |
| | deleted. Projects of this category are presented in the results tables of our reports under |
| | the sections on “deletion variants. |
| site-directed mutation_* | A form of the protein is being overexpressed which has had one or more residues |
| | changed by site directed mutagenesis. |
| domain swap_* | A form of the protein is being overexpressed in which a particular fragment has been |
| | substituted with a region from another protein. |
| KO | Describes a line that harbors a mutation in an Arabidopsis TF at its endogenous locus. In |
| | most cases this is caused by a T-DNA insertion. |
| RNAi (clade) | An RNAi construct designed to knock-down a clade of related genes. |
| RNAi (GS) | An RNAi construct designed to knock-down a specific gene. |
| Promoter-reporter | A construct being used to determine the expression pattern of a gene, or in transient |
| | assay experiments. This would typically be a promoter-GUS or promoter-GFP (or a |
| | derivative of GFP) fusion. |
| Protein-GFP-C-fusion | A translational fusion is being overexpressed in which the TF has GFP rased to the C- |
| | terminus. |
| Protein-YFP-C-fusion | A translational fusion is being overexpressed in which the TF has YFP fused to the C- |
| | terminus. |
| Protein-CFP-C-fusion | A translational fusion is being overexpressed in which the TF has CFP fused to the C- |
| | terminus. |
| 2-components-supTfn- | A translational fusion is being overexpressed in which the TF has a TAP tag (Tandem |
| TAP-C-term | affinity purification epitope, see Rigaut et al., 1999 and Rohila et al., 2004) fused to the |
| | C-terminus. This fusion is being expressed via the two-component system: |
| | promoter::LexA-GAL4; opLexA::TF-TAP. In this case, a stable transgenic line is first |
| | established containing the promoter component and is later supertransformed with the |
| | TF-TAP component). |
| 2-components-supTfn- | A translational fusion is being overexpressed in which the TF has an HA |
| HA-C-term | (hemagglutinin) epitope tag fused to the C-terminus. This fusion is being expressed via |
| | the two-component system: promoter::LexA-GAL4; opLexA::TF-HA. In this case, a |
| | stable transgenic line is first established containing the promoter component and is later |
| | supertransformed with the TF-HA component). |
| 2-components-supTfn- | A translational fusion is being overexpressed in which the TF has an HA |
| HA-N-term | (hemagglutinin) epitope tag fused to the N-terminus. This fusion is being expressed via |
| | the two-component system: promoter::LexA-GAL4; opLexA::HA-TF. In this case, a |
| | stable transgenic line is first established containing the promoter component and is later |
| | supertransformed with the HA-TF component). |
| Double OEX Cross | A transgenic line harboring two different overexpression constructs, created by a genetic |
| | crossing approach. |

*designates any numeric value

### Example II

### Promoter Analysis

A major component of the program is to determine the effects of ectopic expression of transcription factors in a variety of different tissue types, and in response to the onset of stress conditions. Primarily this is achieved by using a panel of different promoters via a two-component system.

Component 1: promoter driver lines (Promoter::LexA/GAL4). In each case, the first component (Promoter::LexA/GAL4) comprises a LexA DNA binding domain fused to a GAL4 activation domain, cloned behind the desired promoter. These constructs are contained within vector backbone pMEN48 (Example III) which also carries a kanamycin resistance marker, along with an opLexA::GFP reporter. The GFP is EGFP, an variant available from Clontech with enhanced signal. EGFP is soluble in the cytoplasm. Transgenic “driver lines” were first obtained containing the Promoter::LexA/GAL4 component. For each promoter driver, a line was selected which showed reproducible expression of the GFP reporter gene in the desired pattern, through a number of generations. We also tested the plants in our standard plate based physiology assays to verify that the tissue specific pattern was not substantially altered by stress conditions. A homozygous population was then established for that line.

Component 2: TF construct (opLexA::TF). Having established a promoter panel, it is possible to overexpress any transcription factor in the precise expression pattern conferred by the driver lines, by super-transforming or crossing in a second construct (opLexA::TF) carrying the TF of interest cloned behind a LexA operator site. In each case this second construct carried a sulfonamide selectable marker and was contained within vector backbone pMEN53 (see Example III).

Arabidopsis promoter driver lines are shown in Table 22 (below).

TABLE 22
Expression patterns conferred by promoters
used for two-component studies.

| | Expression pattern | | Driver |
|---|---|---|---|
| Promoter | conferred | Reference | line used |
| 35S | Constitutive | Odell et al. (1985) | line 17 |
| SUC2 | Vascular/Phloem | Truernit and Sauer | line 6 |
| | | (1995) | |
| ARSK1 | Root | Hwang and | line 8 |
| | | Goodman (1995) | |
| CUT1 | Shoot epidermal/guard cell | Kunst et al. (2000) | line 2 |
| | enhanced | | |
| RBCS3 | Photosynthetic tissue | Wanner and | line 4 |
| | | Gruissem (1991) | |
| RD29A* | Drought/Cold/ABA | Yamaguchi- | lines 2 |
| | inducible | Shinozaki and | and 5 |
| | | Shinozaki (1993) | |
| LTP1 | Shoot epidermal/trichome | Thoma et al. | line 1 |
| | enhanced | (1994) | |
| RSI1 | Root meristem and root | Taylor and | line 34 |
| | vascular | Scheuring (1994) | |
| AP1 | Flower primordia/Flower | Hempel et al. | line 16 |
| | | (1997); Mandel et | |
| | | al. (1992) | |
| STM | Meristems | Long and Barton | lines 5 |
| | | (2000); Long et al. | and 10 |
| | | (1996) | |
| AS1 | Primordia and young | Byrne et al. (2000) | line 1026 |
| | organs | | |

Notes:
Two different RD29A promoter lines, lines 2 and 5, were in use. Line 2 has a higher level of background expression than line 5. Expression from the line 2 promoter was expected to produce constitutive moderate basal transcript levels of any gene controlled by it, and to generate an increase in levels following the onset of stress. In contrast, line 5 was expected to produce lower basal levels and a somewhat sharper up-regulation of any gene under its control, following the onset of stress. Although RD29A exhibits up-regulation in response to cold and drought in mature tissues, this promoter produces relatively highly levels of expression in embryos and young seedlings.

Validation of the Promoter-driver line patterns. To demonstrate that each of the promoter driver lines could generate the desired expression pattern of a second component target at an independent locus arranged in trans, crosses were made to an opLexA::GUS line. Typically, it was confirmed that the progeny exhibited GUS activity in an equivalent region to the GFP seen in the parental promoter driver line. However, GFP can move from cell-to-cell early in development and in meristematic tissues, and hence patterns of GFP in these tissues do not strictly report gene expression.

Given that the two-component combinations for the Lead Advancement program were obtained by a supertransformation approach, we performed a separate set of control experiments in which an opLexA::GUS reporter construct was supertransformed into each of the promoter driver lines. The aim was to verify that the expression pattern was maintained for the majority of independent insertion events for the target gene. For each of the promoter lines, the pattern was maintained in the majority of supertransformants, except in the case of the SUC2 driver line. For unknown reasons, the expression from this driver line was susceptible to silencing on supertransformation. It remains to be determined whether this was a general facet of SUC2 promoter itself, following supertransformation, or whether the effect was confined specifically to the line initially selected for supertransformation. We have are therefore establishing a new SUC2 driver line for use in two-component supertransformation approaches, as well as cloning the SUC2 promoter into a transformation vector backbone to allow its use via direct-promoter fusion to different TFs. To test the promoter fragment cloned in this direct promoter-fusion vector, we created both SUC2::GFP and SUC2::GUS promoter-reporter constructs in the vector as controls. In each case, the expected expression pattern was obtained in the majority of independent transformants obtained. Preliminary results indicate that the direct fusion lines are predictable, with regard to pattern. However, expression levels are quite variable, with many lines having very low levels of vascular expression. This may suggest that the SUC2 promoter is relatively susceptible to gene silencing.

It is clear that the 35S promoter induces much higher levels of expression compared to the other promoters presently in use.

### Example III

### Vector and Cloning Information

### Vector and Cloning Information: Expression Vectors.

A list of constructs (PIDs) included in this application, indicating the promoter fragment that was used to drive the transgene, along with the cloning vector backbone, is provided in Table 23. Compilations of the sequences of promoter fragments (SEQ ID NO: 927 to 937) and the expressed transgene sequences within the PIDs (SEQ ID NO: 421 to 900) are provided in the Sequence Listing. Plant Expression vectors that have been generated are summarized in the following table and more detailed description are provided below.

TABLE 23
Summary of Plant Expression Vectors

| Construct | | | | Description of the |
|---|---|---|---|---|
| Name | Class | Construct Description | Selection | included sequence |
| pMEN001 | 35S | 35S::MCS::Nos | prNOS::NPTII::Nos | T-DNA segment |
| | expression | | | (SEQ ID NO: 901) |
| | vector | | | |
| pMEN20 | 35S | 35S::MCS::E9 | 35S::NPTII::Nos | 35S::MCS::E9 |
| | expression | | | (SEQ ID NO: 902) |
| | vector | | | |
| pMEN65 | 35S | 35S::MCS::E9 | prNOS::NPTII::Nos | T-DNA segment |
| | expression | | | (SEQ ID NO: 903) |
| | vector | | | |
| pMEN1963 | 35S | 35S::attR1::CAT::ccdB::attR2:: | prNOS::NPTII::Nos | T-DNA segment |
| | expression | E9 | | (SEQ ID NO: 904) |
| | vector | | | |
| P5360 | 35S | 35S::MCS::E9 | 35S::NPmito::Sulf:: | T-DNA segment |
| | expression | | Nos | (SEQ ID NO: 905) |
| | vector | | | |
| P5375 | 2-component | MCS::m35S::oEnh::LexAGal4:: | 35S::NPTII::Nos | MCS::m35S::oEnh:: |
| (pMEN48) | driver vector | E9 (opLexA::GFP::E9) | | LexAGal4 |
| | | | | (SEQ ID NO: 906) |
| P5386 | 2-component | 35S::oEnh::LexAGal4::E9 | 35S::NPTII::Nos | 35S::oEnh::LexAGal4 |
| (pMEN57) | driver vector | (opLexA::GFP::E9) | | (SEQ ID NO: 907) |
| P5381 | 2-component | opLexA::MCS::E9 | 35S::NPmito::Sulf:: | opLexA::MCS |
| (pMEN53) | target vector | | Nos | (SEQ ID NO: 908) |
| P5480 | 2-component | opLexA::attR1::CAT::ccdB:: | 35S::NPmito::Sulf:: | opLexA::attR1::CAT:: |
| (pMEN256) | target vector | attR2::E9 | Nos | ccdB::attR2::E9 |
| | | | | (SEQ ID NO: 909) |
| P25420 | 2-component | opLexA::MCS::(9A)TAP::E9 | 35S::NPmito::Sulf:: | MCS::(9A)TAP |
| | target vector | | Nos | (SEQ ID NO: 910) |
| P25976 | 2-component | opLexA::12xHA(10A)::MCS:: | 35S::NPmito::Sulf:: | 12xHA(10A)::MCS |
| | target vector | E9 | Nos | (SEQ ID NO: 911) |
| P25461 | 2-component | opLexA::MCS::(10A)12xHA:: | 35S::NPmito::Sulf:: | MCS::(10A)12xHA |
| | target vector | E9 | Nos | (SEQ ID NO: 912) |
| P21171 | GR fusion | 35S::MCS::GR::E9 | prNOS::NPTII::Nos | MCS::GR |
| | vector | | | (SEQ ID NO: 913) |
| P21173 | GR fusion | 35S::GR::MCS::E9 | prNOS::NPTII::Nos | GR::MCS |
| | vector | | | (SEQ ID NO: 914) |
| P21172 | GR-HA | 35S::MCS::GR::6xHA::E9 | prNOS::NPTII::Nos | MCS::GR::6xHA |
| | fusion | | | (SEQ ID NO: 915) |
| | vector | | | |
| P21174 | GR-HA | 35S::GR::MCS::6xHA::E9 | prNOS::NPTII::Nos | GR::MCS::6xHA |
| | fusion | | | (SEQ ID NO: 916) |
| | vector | | | |
| P5425 | GAL4 fusion | 35S::G40::GAL4 | prNOS::NPTII::Nos | G40::GAL4 |
| (pMEN201) | vector | | | (SEQ ID NO: 917) |
| P21195 | GAL4 fusion | 35S::Gal4::MCS::E9 | prNOS::NPTII::Nos | Gal4::MCS |
| | vector | | | (SEQ ID NO: 918) |
| P21378 | GAL4 fusion | 35S::MCS::Gal4::E9 | prNOS::NPTII::Nos | MCS::Gal4 |
| | vector | | | (SEQ ID NO: 919) |
| P25799 | GFP fusion | 35S::MCS::GFP::E9 | prNOS::NPTII::Nos | MCS::GFP |
| | vector | | | (SEQ ID NO: 920) |
| P25801 | CFP fusion | 35S::MCS::(9A)CFP::E9 | prNOS::NPTII::Nos | MCS::(9A)CFP |
| | vector | | | (SEQ ID NO: 921) |
| P25800 | YFP fusion | 35S::MCS::(9A)YFP::E9 | prNOS::NPTII::Nos | MCS::(9A)YFP |
| | vector | | | (SEQ ID NO: 922) |
| P32122 | Promoter | MCS::GFP::E9 | prNOS::NPTII::Nos | MCS::GFP |
| | reporter | | | (SEQ ID NO: 923) |
| | vector | | | |
| P21142 | Promoter | MCS::intGUS::E9 | prNOS::NPTII::Nos | MCS::intGUS |
| | reporter | | | (SEQ ID NO: 924) |
| | vector | | | |
| P25755 | Promoter | MCS::YFPLTI6b::E9 | prNOS::NPTII::Nos | MCS::YFPLTI6b |
| | reporter | | | (SEQ ID NO: 925) |
| | vector | | | |
| P21103 | RNAi | 35S::MCS::PDK::MCS::E9 | prNOS::NPTII::Nos | MCS::PDK::MCS |
| | vector | | | (SEQ ID NO: 926) |

Table 24 Legend: 10A: 10× alanine spacer; 12×HA: twelve repeats of the HA epitope tag; attR1/attR2: Gateway recombination sequence; CAT: chloramphenicol resistance; ccdb: counter selectable marker; E9: E9 3-prime UTR; GR: glucocorticoid receptor; intGUS: GUS reporter gene with an intron; LexAGal4 DNA binding protein; MCS: multiple cloning site; Nos: Nopaline synthase 3-prime UTR; NPmito: mitochondrial targeting sequence; oEnh: Omega enhancer; prNOS: Nopaline synthase promoter; NPTII: Kanamycin resistance; YFP/CFP: GFP reporter protein variant; YFPLTI6b: YFP fusion for membrane localization

Other Construct Element Sequences, which may be found in the table below and in the Sequence Listing, include: the 35S promoter (35S), the NOS promoter (prNOS), the minimal 35S (m35S), the omega Enhancer (oEnh), the Nos terminator (Nos), the E9 terminator (E9), and the NPmito::Sulfonamide element.

TABLE 24

Other Construct Element Sequences

| | |
|---|---|
| Element | Sequence |

| | |
|---|---|
| 35S | gcggattccattgcccagctatctgtcactttattgtgaagatagtgaaaaagaaggtggctcctacaaatgccatcattgcgataaagga |
| promoter | aaggccatcgttgaagatgcctctgccgacagtggtcccaaagatggacccccacccacgaggagcatcgtggaaaaagaagacgttccaa |
| (35S) | ccacgtcttcaaagcaagtggattgatgtgatggtccgattgagacttttcaacaaagggtaatatccggaaacctcctcggattccattg |
| | cccagctatctgtcactttattgtgaagatagtggaaaaggaaggtggctcctacaaatgccatcattgcgataaaggaaaggccatcgtt |
| | gaagatgcctctgccgacagtggtcccaaagatggacccccacccacgaggagcatcgtggaaaaagaagacgttccaaccacgtcttcaa |
| | agcaagtggattgatgtgatatctccactgacgtaagggatgacgcacaatcccactatccttcgcaagacccttcctctatataaggaag |
| | ttcatttcatttggagaggacacgctga |
| NOS | tcgagatcatgagcggagaattaagggagtcacgttatgacccccgccgatgacgcgggacaagccgttttacgtttggaactgacagaac |
| promoter | cgcaacgttgaaggagccactcagccgcgggtttctggagtttaatgagctaagcacatacgtcagaaaccattattgcgcgttcaaaagt |
| (prNOS) | cgcctaaggtcactatcagctagcaaatatttcttgtcaaaaatgctccactgacgttccataaattcccctcggtatccaattagagtct |
| | catattcactctcaatccaaataatctgcaccggatctggatcgtttcgc |
| minimal | cgcaagacccttcctctatataaggaagttcatttcatttggagaggacacgctc |
| 35S | |
| (m355) | |
| omega | atttttacaacaattaccaacaacaacaaacaacaaacaacattacaattacatttacaattacca |
| Enhancer | |
| (oEnh) | |
| Nos | gcgggactctggggttcgaaatgaccgaccaagcgacgcccaacctgccatcacgagatttcgattccaccgccgccttctatgaaaggtt |
| terminator | gggcttcggaatcgttttccgggacgccggctggatgatcctccagcgcggggatctcatgctggagttcttcgcccacgggatctctgcg |
| (Nos) | gaacaggcggtcgaaggtgccgatatcattacgacagcaacggccgacaagcacaacgccacgatcctgagcgacaatatgatcgggcccg |
| | gcgtccacatcaacggcgtcggcggcgactgcccaggcaagaccgagatgcaccgcgatatcttgctgcgttcggatattttcgtggagtt |
| | cccgccacagacccggatgatccccgatcgttcaaacatttggcaataaagtttcttaagattgaatcctgttgccggtcttgcgatgatt |
| | atcatataatttctgttgaattacgttaagcatgtaataattaacatgtaatgcatgacgttatttatgagatgggtttttatgattagag |
| | tcccgcaattatacatttaatacgcgatagaaaacaaaatatagcgcgcaaactaggataaattatcgcgcgcggtgtcatctatgttact |
| | agatcggg |
| E9 | gatcctctagctagagctttcgttcgtatcatcggtttcgacaacgttcgtcaagttcaatgcatcagtttcattgcgcacacaccagaat |
| terminator | cctactgagtttgagtattatggcattgggaaaactgtttttcttgtaccatttgttgtgcttgtaatttactgtgttttttattcggttt |
| (E9) | tcgctatcgaactgtgaaatggaaatggatggagaagagttaatgaatgatatggtccttttgttcattctcaaattaatattatttgttt |
| | tttctcttatttgttgtgtgttgaatttgaaattataagagatatgcaaacattttgtttgagtaaaaatgtgtcaaatcgtggcctctaa |
| | tgaccgaagttaatatgaggagtaaaacacttgtagttgtaccattatgcttattcactaggcaacaaatatattttcagacctagaaaag |
| | ctgcaaatgttactgaatacaagtatgtcctcttgtgttttagacatttatgaactttcctttatgtaattttccagaatccttgtcagat |
| | tctaatcattgctttataattatagttatactcatggatttgtagttgagtatgaaaatattttttaatgcattttatgacttgccaattg |
| | attgacaacatgcatcaatcgacctgcagccactcgaagcggccggccgccac |
| NPmito::Sul | agctcatttttacaacaattaccaacaacaacaaacaacaaacaacattacaattacatttacaattatcgatggcttctcggaggcttct |
| fonamide | cgcctctctcctccgtcaatcggctcaacgtggcggcggtctaatttcccgatcgttaggaaactccatccctaaatccgcttcacgcgcc |
| | tcttcacgcgcatcccctaagggattcctcttaaaccgcgccgtacagtacgctacctccgcagcggcaccggcatctcagccatcaacac |
| | caccaaagtccggcagtgaaccgtccggaaaattaccgatgagttcaccggcgctggttcgatcggtgccatggataaatcgctcatcatt |
| | ttcggcatcgtcaacataacctcggacagtttctccgatggaggccggtatctggcgccagacgcagccattgcgcaggcgcgtaagctga |
| | tggccgagggggcagatgtgatcgacctcggtccggcatccagcaatcccgacgccgcgcctgtttcgtccgacacagaaatcgcgcgtat |
| | cgcgccggtgctggacgcgctcaaggcagatggcattcccgtctcgctcgacagttatcaacccgcgacgcaagcctatgccttgtcgcgt |
| | ggtgtggcctatctcaatgatattcgcggttttccagacgctgcgttctatccgcaattggcgaaatcatctgccaaactcgtcgttatgc |
| | attcggtgcaagacgggcaggcagatcggcgcgaggcacccgctggcgacatcatggatcacattgcggcgttctttgacgcgcgcatcgc |
| | ggcgctgacgggtgccggtatcaaacgcaaccgccttgtccttgatcccggcatggggttttttctgggggctgctcccgaaacctcgctc |
| | tcggtgctggcgcggttcgatgaattgcggctgcgcttcgatttgccggtgcttctgtctgtttcgcgcaaatcctttctgcgcgcgctca |
| | caggccgtggtccgggggatgtcggggccgcgacactcgctgcagagcttgccgccgccgcaggtggagctgacttcatccgcacacacga |
| | gccgcgccccttgcgcgacgggctggcggtattggcggcgctgaaagaaaccgcaaggattcgttaa |

### 35S Expression Vectors

pMEN001 is a derivative of pBI121 in which kanamycin resistance gene is driven by the Nos promoter. pMEN001 was used for the initial cloning of a number of Arabidopsis transcription factors. (Sequence of pMEN001 polylinker=SEQ ID NO: 901)

pMEN20 is an earlier version of pMEN65 in which the kanamycin resistance gene is driven by the 35S promoter rather than the nos promoter. It is the base vector for P5381, P5425, P5375, and some of the older Arabidopsis transcription factor overexpression constructs. (Sequence of pMEN20 polylinker=SEQ ID NO: 902)

pMEN65 is a derivative of pMON10098. The only differences between pMEN65 and pMON10098 are the polylinker and the fact that the kanamycin gene is driven by the nos promoter. pMEN65 is the base vector for the majority of the transcription factor overexpression clones. (Sequence of pMEN65=SEQ ID NO: 903);

| pMEN65 primers: | |
|---|---|
| 35S | gcaagtggattgatgtgatatc |
| O5183 | tttggagaggacacgctgacaa |
| O6344 | atccggtacgaggcctgtctagag |
| E9 | caaactcagtaggattctggtgtgt |

```
pMEN65 polylinker:
                                  gcaagtggattgatgtgatatc->primer 35S
CCAACCACGTCTTCAAAGCAAGTGGATTGATGTGATATCTCCACTGACGTAAGGGATGACGCACAATCCCACTATCCTTCGCAAGAC
CCT
GGTTGGTGCAGAAGTTTCGTTCACCTAACTACACTATAGAGGTGACTGCATTCCCTACTGCGTGTTAGGGTGATAGGAAGCGTTCTG
GGA
                                                    tttggagaggacacgctgacaa->primer O5183
TCCTCTATATAAGGAAGTTCATTTCATTTGGAGAGGACACGCTGACAAGCTGACTCTAGCAGATCTGGTACCGTCGACGGTGAGCTC
CGC
AGGAGATATATTCCTTCAAGTAAAGTAAACCTCTCCTGTGCGACTGTTCGACTGAGATCGTCTAGACCATGGCAGCTGCCACTCGAG
GCG
                                                                                                              --------pMEN65 MCS------------
GGCCGCTCTAGACAGGCCTCGTACCGGATCCTCTAGCTAGAGCTTTCGTTCGTATCATCGGTTTCGACAACGTTCGTCAAGTTCAAT
GCA
CCGGCGAGATCTGTCCGGAGCATGGCCTAGGAGATCGATCTCGAAAGCAAGCATAGTAGCCAAAGCTGTTGCAAGCAGTTCAAGTTA
CGT
      <-gagatctgtccggagcatggccta primer O6344
TCAGTTTCATTGCGCACACACCAGAATCCTACTGAGTTTGAGTATTATGGCATT
AGTCAAAGTAACGCGTGTGTGGTCTTAGGATGACTCAAACTCATAATACCGTAA
                          <-tgtgtggtcttaggatgactcaaac primer E9
```

pMEN1963 is a derivative of pMEN65 with Gateway attR sites flanking the ccdB gene, a counter-selectable marker. This vector is used to receive an insert flanked by attL sites from a Gateway entry clone. It was the base vector for many of the Arabidopsis transcription factor overexpression clones. Sequence of pMEN1963 SEQ ID NO: 904)

P5360 is a derivative of pMEN65 in which the kanamycin resistance gene was replaced by a mitochondrial-targeted sulfonamide resistance gene. Sequence of P5360=SEQ ID NO: 905)

### Two-Component Vectors

P5375 (also called pMEN48) is the 2-component base vector used to express the LexA:GAL4 chimeric activator under different promoters. It contains a multiple cloning site in front of the LexA:GAL4 gene, followed by the GFP reporter gene under the control of the LexA operator. It has a pMEN20 backbone, and carries kanamycin resistance under the 35S promoter. (Sequence of P5375 insert=SEQ ID NO: 906)

P5386 (also called pMEN57) is a derivative of P5375 in which the 35S promoter from pBI121 is cloned into the HindIII and NotI sites of p5375. It drives expression of the LexA:GAL4 activator under the 35S promoter. (Sequence of P5386 insert=SEQ ID NO: 907)

P5381 (also called pMEN53) is the 2-component base vector that was used to express genes under the control of the LexA operator. It contains eight tandem LexA operators from plasmid p8op-lacZ (Clontech) followed by a polylinker. The plasmid carries a sulfonamide resistance gene driven by the 35S promoter. (Sequence of P5381 LexAOp and polylinker regions=SEQ ID NO: 908)

P5480 (also called pMEN256) is a derivative of P5381 in which the multiple cloning site is replaced with Gateway attR sites flanking the ccdB gene. This vector was used to receive an insert flanked by attL sites from a Gateway entry clone. (Sequence of P5480 (pMEN256) (opLexA::attR1::CAT::ccdB::attR2::E9)=SEQ ID NO: 909)

P25420 is the based vector for the development of C-term TAP fusion. The vector includes a 10-alanine spacer segment between the gene of interest and the TAP element. This is a 2-component vector with the LexA operator. (Sequence of P25420 insert=SEQ ID NO: 910)

P25976 is the based vector for the development of N-term TAP fusion. The vector includes a 10-alanine spacer segment between the gene of interest and the TAP element. This is a 2-component vector with the LexA operator (Sequence of P25976 insert=SEQ ID NO: 911)

P25461 is the based vector for the development of C-term 12×HA fusion. The vector includes a 10-alanine spacer segment between the gene of interest and the 12×HA element. This is a 2-component vector with the LexA operator. (Sequence of P25461 insert=SEQ ID NO: 912)

### Fusion Vectors

P21171 is the backbone vector for creation of C-terminal glucocorticoid receptor fusion constructs. The GR hormone binding domain minus the ATG was amplified and cloned into pMEN65 with NotI and XbaI. To create gene fusions, the gene of interest was amplified using a 3′ primer that ends at the last amino acid codon before the stop codon. The PCR product can then be cloned into the SalI and NotI sites. (Sequence of P21171 GR coding sequence and polylinker=SEQ ID NO: 913)

P21173 is the backbone vector for creation of N-terminal glucocorticoid receptor fusion constructs. The GR hormone binding domain including the ATG was amplified and cloned into pMEN65 with BglII and KpnI. To create gene fusions, the gene of interest was amplified using a primer that starts at the second amino acid and has added the KpnI or SalI and NotI sites. The PCR product was then cloned into the KpnI or SalI and NotI sites of P21173, taking care to maintain the reading frame. (Sequence of P21173 GR coding sequence and polylinker ═SEQ ID NO: 914)

P21172 is the based vector for the development of N-terminal glucocorticoid receptor fusion constructs with an N-terminal HA epitope tag. (Sequence of P21172 insert=SEQ ID NO: 915)

P21174 is the based vector for the development of C-terminal glucocorticoid receptor fusion constructs with an N-terminal HA epitope tag. (Sequence of P21174 insert=SEQ ID NO: 916)

P21195 is the backbone vector for creation of N-terminal GAL4 activation domain protein fusions. It was created by inserting the GAL4 activation domain into the BglII and KpnI sites of pMEN65. To create gene fusions, the gene of interest was amplified using a primer that starts at the second amino acid and has added the KpnI or SalI and NotI sites. The PCR product was then cloned into the KpnI or SalI and NotI sites of P21195, taking care to maintain the reading frame. (Sequence of P21195 GAL4 activation domain and polylinker=SEQ ID NO: 918)

P21378 was constructed to serve as a backbone vector for creation of C-terminal GAL4 activation domain fusions. However, P5425 (see below) was also used as a backbone construct. P21378 was constructed by amplification of the GAL4 activation domain and insertion of this domain into the NotI and XbaI sites of pMEN65. To create gene fusions, the gene of interest was amplified using a 3′ primer that ends at the last amino acid codon before the stop codon. The PCR product can then be cloned into the SalI and NotI sites. (Sequence of P21378 GAL4 activation domain and polylinker=SEQ ID NO: 919)

P5425 (also called pMEN201) is a derivative of pMEN20 that carries a CBF1:GAL4 fusion. To construct other GAL4 fusions, the CBF1 gene was removed with SalI or Kpn1 and EcoRI. The gene of interest was amplified using a 3′ primer that ended at the last amino acid codon before the stop codon and contained an EcoRI or Mfe1 site. The product was inserted into these SalI or KpnI and EcoRI sites, taking care to maintain the reading frame. (Sequence of P5425 (pMEN201)=SEQ ID NO: 917)

P25799 is the based vector for the development of C-terminal GFP fusion constructs. (Sequence of P25799 insert=SEQ ID NO: 920)

P25801 is the based vector for the development of C-terminal CFP fusion constructs. The vector includes a 10-alanine spacer segment between the gene of interest and the CFP element. (Sequence of P25801 insert=SEQ ID NO: 921)

P25800 is the based vector for the development of C-terminal YFP fusion constructs. The vector includes a 10-alanine spacer segment between the gene of interest and the YFP element. (Sequence of P25800 insert=SEQ ID NO: 922)

### Promoter-Reporter Vectors

P32122 is the based vector for the development of GFP reporter constructs. (Sequence of P32122 insert=SEQ ID NO: 923)

P21142 is the based vector for the development of GUS reporter constructs. (Sequence of P21142 insert SEQ ID NO: 924)

P25755 is the based vector for the development of membrane-anchored YFP reporter constructs. (Sequence of P25755 insert=SEQ ID NO: 925)

### RNAi Vector

P21103 is the backbone vector for the creation of RNAi constructs. The PDK intron from pKANNIBAL (Wesley et al. (2001)) was amplified and cloned into the SalI and NotI sites of pMEN65. An EcoRI site was included in the 5′ primer between the SalI site and the Pdk intron sequence. RNAi constructs were generated as follows:

The target sequence was amplified with primers with the following restriction sites:

5′ primer: BamHI and SalI

3′ primer: XbaI and EcoRI

A sense fragment was inserted in front of the Pdk intron using SalI and EcoRI to generate an intermediate vector.

The same fragment was then subcloned into the intermediate vector behind the PDK intron in the antisense orientation using XbaI and EcoRI.

Target sequences were selected to be 100 bp long or longer. For constructs designed against a clade rather than a single gene, the target sequences have at least 85% identity to all clade members. Where it is not possible to identity a single 100 bp sequence with 85% identity to all clade members, hybrid fragments composed of two shorter sequences were used. Sequence of P21103 polylinker and PDK intron=SEQ ID NO: 926)

Cloning methods. The sequence of each clone used in this report is presented with the results of the phenotypic screens, or in an appendix in the case of clones used in the TFSeeker™ assay.

Arabidopsis transcription factor clones used in this report were created in one of three ways: isolation from a library, amplification from cDNA, or amplification from genomic DNA. The ends of the Arabidopsis transcription factor coding sequences were generally confirmed by RACE PCR or by comparison with public cDNA sequences before cloning.

Clones of transcription factor orthologs from rice, maize, and soybean presented in this report were all made by amplification from cDNA. The ends of the coding sequences were predicted based on homology to Arabidopsis or by comparison to public and proprietary cDNA sequences; RACE PCR was not done to confirm the ends of the coding sequences. For cDNA amplification, we used KOD Hot Start DNA Polymerase (Novagen), in combination with 1M betaine and 3% DMSO. This protocol was found to be successful in amplifying cDNA from GC-rich species such as rice and corn, along with some non-GC-rich species such as soybean and tomato, where traditional PCR protocols failed. Primers were designed using at least 30 bases specific to the target sequence, and were designed close to, or overlapping, the start and stop codons of the predicted coding sequence.

Clones were fully sequenced. In the case of rice, high-quality public genomic sequences were available for comparison, and clones with sequence changes that result in changes in amino acid sequence of the encoded protein were rejected. For corn and soy, however, it was often unclear whether sequence differences represent an error or polymorphism in the source sequence or a PCR error in the clone. Therefore, in the cases where the sequence of the clone we obtained differed from the source sequence, a second clone was created from an independent PCR reaction. If the sequences of the two clones agreed, then the clone was accepted as a legitimate sequence variant.

Transformation. Agrobacterium strain ABI was used for all plant transformations. This strain is chloramphenicol, kanamycin and gentamicin resistant.

### Example IV

### GR Line Analysis

A one- or two-component approach was used to generate dexamethasone inducible lines used, as detailed below.

One-component dex-inducible lines. In the one-component system, direct-GR fusion constructs are made for overexpression of a TF with a glucocorticoid receptor fusion at either its N or C terminal end.

Two-component dex-inducible lines. For the two component strategy, a kanamycin resistant 35S::LexA-GAL4-TA driver line was established and was then supertransformed with opLexA::TF constructs (carrying a sulfonamide resistance gene) for each of the transcription factors of interest.

Establishment of the 35S::LexA-GAL4-TA driver line. Approximately one hundred 35S::LexA-GAL4-TA independent driver lines containing construct pMEN262 (also known as P5486) were generated at the outset of the experiment. Primary transformants were selected on kanamycin plates and screened for GFP fluorescence at the seedling stage. Any lines that showed constitutive GFP activity were discarded. At 10 days, lines that showed no GFP activity were then transferred onto MS agar plates containing dexamethasone (5 μM). Lines were that showed strong GFP activation by 2-3 days following the dexamethasone treatments were marked for follow-up in the T2 generation. Following similar experiments in the T2 generation, a single line, 65, was selected for future studies. Line 66 lacked any obvious background expression and all plants showed strong GFP fluorescence following dexamethasone application. A homozygous population for line 65 was then obtained, re-checked to ensure that it still exhibited induction following dexamethasone application, and bulked. 35S::LexA-GAL4-TA line 65 was also crossed to an opLexA::GUS line to demonstrate that it could drive activation of targets arranged in trans.

### Example V

### Transformation

Transformation of Arabidopsis was performed by an Agrobacterium-mediated protocol based on the method of Bechtold and Pelletier (1998). Unless otherwise specified, all experimental work was done using the Columbia ecotype.

Plant preparation. Arabidopsis seeds were sown on mesh covered pots. The seedlings were thinned so that 6-10 evenly spaced plants remained on each pot 10 days after planting. The primary bolts were cut off a week before transformation to break apical dominance and encourage auxiliary shoots to form. Transformation was typically performed at 4-5 weeks after sowing.

Bacterial culture preparation. Agrobacterium stocks were inoculated from single colony plates or from glycerol stocks and grown with the appropriate antibiotics and grown until saturation. On the morning of transformation, the saturated cultures were centrifuged and bacterial pellets were re-suspended in Infiltration Media (0.5×MS, 1×B5 Vitamins, 5% sucrose, 1 mg/ml benzylaminopurine riboside, 200 μl/L Silwet L77) until an A600 reading of 0.8 is reached.

Transformation and seed harvest. The Agrobacterium solution was poured into dipping containers. All flower buds and rosette leaves of the plants were immersed in this solution for 30 seconds. The plants were laid on their side and wrapped to keep the humidity high. The plants were kept this way overnight at 4° C. and then the pots were turned upright, unwrapped, and moved to the growth racks.

The plants were maintained on the growth rack under 24-hour light until seeds were ready to be harvested. Seeds were harvested when 80% of the siliques of the transformed plants are ripe (approximately 5 weeks after the initial transformation). This seed was deemed T0 seed, since it was obtained from the T0 generation, and was later plated on selection plates (either kanamycin or sulfonamide, see Example VI). Resistant plants that were identified on such selection plates comprised the T1 generation.

### Example VI

### Morphology

Morphological analysis was performed to determine whether changes in transcription factor levels affect plant growth and development. This was primarily carried out on the T1 generation, when at least 10-20 independent lines were examined. However, in cases where a phenotype required confirmation or detailed characterization, plants from subsequent generations were also analyzed.

Primary transformants were selected on MS medium with 0.3% sucrose and 50 mg/l kanamycin. T2 and later generation plants were selected in the same manner, except that kanamycin was used at 35 mg/l. In cases where lines carry a sulfonamide marker (as in all lines generated by super-transformation), seeds were selected on MS medium with 0.3% sucrose and 1.5 mg/l sulfonamide. KO lines were usually germinated on plates without a selection. Seeds were cold-treated (stratified) on plates for 3 days in the dark (in order to increase germination efficiency) prior to transfer to growth cabinets. Initially, plates were incubated at 22° C. under a light intensity of approximately 100 microEinsteins for 7 days. At this stage, transformants were green, possessed the first two true leaves, and were easily distinguished from bleached kanamycin from bleached kanamycin or sulfonamide-susceptible seedlings. Resistant seedlings were then transferred onto soil (Sunshine potting mix). Following transfer to soil, trays of seedlings were covered with plastic lids for 2-3 days to maintain humidity while they became established. Plants were grown on soil under fluorescent light at an intensity of 70-95 microEinsteins and a temperature of 18-23° C. Light conditions consisted of a 24-hour photoperiod unless otherwise stated. In instances where alterations in flowering time was apparent, flowering may was re-examined under both 12-hour and 24-hour light to assess whether the phenotype was photoperiod dependent. Under our 24-hour light growth conditions, the typical generation time (seed to seed) was approximately 14 weeks.

Because many aspects of Arabidopsis development are dependent on localized environmental conditions, in all cases plants were evaluated in comparison to controls in the same flat. As noted below, controls for transgenic lines were wild-type plants, plants overexpressing CBF4, or transgenic plants harboring an empty transformation vector selected on kanamycin or sulfonamide. Careful examination was made at the following stages: seedling (1 week), rosette (2-3 weeks), flowering (4-7 weeks), and late seed set (8-12 weeks). Seed was also inspected. Seedling morphology was assessed on selection plates. At all other stages, plants were macroscopically evaluated while growing on soil. All significant differences (including alterations in growth rate, size, leaf and flower morphology, coloration and flowering time) were recorded, but routine measurements were not be taken if no differences were apparent. In certain cases, stem sections were stained to reveal lignin distribution. In these instances, hand-sectioned stems were mounted in phloroglucinol saturated 2M HCl (which stains lignin pink) and viewed immediately under a dissection microscope.

Note that for a given project (gene-promoter combination, GAL4 fusion lines, RNAi lines etc.), ten lines were typically examined in subsequent plate based physiology assays.

### Example VII

### Physiology Experimental Methods

Plate Assays. Twelve different plate-based physiological assays (shown below), representing a variety of drought-stress related conditions, were used as a pre-screen to identify top performing lines from each project (i.e. lines from transformation with a particular construct), that may be tested in subsequent soil based assays. Typically, ten lines were subjected to plate assays, from which the best three lines were selected for subsequent soil based assays. However, in projects where significant stress tolerance was not obtained in plate based assays, lines were not submitted for soil assays.

In addition, some projects were subjected to nutrient limitation studies. A nutrient limitation assay was intended to find genes that allow more plant growth upon deprivation of nitrogen. Nitrogen is a major nutrient affecting plant growth and development that ultimately impacts yield and stress tolerance. These assays monitor primarily root but also rosette growth on nitrogen deficient media. In all higher plants, inorganic nitrogen is first assimilated into glutamate, glutamine, aspartate and asparagine, the four amino acids used to transport assimilated nitrogen from sources (e.g. leaves) to sinks (e.g. developing seeds). This process is regulated by light, as well as by C/N metabolic status of the plant. We used a C/N sensing assay to look for alterations in the mechanisms plants use to sense internal levels of carbon and nitrogen metabolites which could activate signal transduction cascades that regulate the transcription of N-assimilatory genes. To determine whether these mechanisms are altered, we exploited the observation that wild-type plants grown on media containing high levels of sucrose (3%) without a nitrogen source accumulate high levels of anthocyanins. This sucrose induced anthocyanin accumulation can be relieved by the addition of either inorganic or organic nitrogen. We used glutamine as a nitrogen source since it also serves as a compound used to transport N in plants.

Germination assays. NaCl (150 mM), mannitol (300 mM), sucrose (9.4%), ABA (0.3 μM), Heat (32° C.), Cold (8° C.), —N is basal media minus nitrogen plus 3% sucrose and —N/+Gln is basal media minus nitrogen plus 3% sucrose and 1 mM glutamine.

Growth assays. Growth assays consisted of severe dehydration (plate-based desiccation or drought), heat (32° C. for 5 days followed by recovery at 22° C.), chilling (8° C.), root development (visual assessment of lateral and primary roots, root hairs and overall growth). For the nitrogen limitation assay, all components of MS medium remained constant except nitrogen was reduced to 20 mg/L of NH₄NO₃. Note that 80% MS had 1.32 g/L NH₄NO₃ and 1.52 g/L KNO₃.

Unless otherwise stated, all experiments were performed with the Arabidopsis thaliana ecotype Columbia (col-0). Assays were usually performed on non-selected segregating T2 populations (in order to avoid the extra stress of selection). Control plants for assays on lines containing direct promoter-fusion constructs were Col-0 plants transformed an empty transformation vector (pMEN65). Controls for 2-component lines (generated by supertransformation) were the background promoter-driver lines (i.e. promoter::LexA-GAL4TA lines), into which the supertransformations were initially performed.

All assays were performed in tissue culture. Growing the plants under controlled temperature and humidity on sterile medium produced uniform plant material that had not been exposed to additional stresses (such as water stress) which could cause variability in the results obtained. All assays were designed to detect plants that were more tolerant or less tolerant to the particular stress condition and were developed with reference to the following publications: Jang et al. (1997), Smeekens (1998), Liu and Zhu (1997), Saleki et al. (1993), Wu et al. (1996), Zhu et al. (1998), Alia et al. (1998), Xin and Browse, (1998), Leon-Kloosterziel et al. (1996). Where possible, assay conditions were originally tested in a blind experiment with controls that had phenotypes related to the condition tested.

### Procedures

Prior to plating, seed for all experiments were surface sterilized in the following manner: (1) 5 minute incubation with mixing in 70% ethanol, (2) 20 minute incubation with mixing in 30% bleach, 0.01% triton-X 100, (3) 5× rinses with sterile water, (4) Seeds were re-suspended in 0.1% sterile agarose and stratified at 4° C. for 3-4 days.

All germination assays follow modifications of the same basic protocol. Sterile seeds were sown on the conditional media that had a basal composition of 80% MS+Vitamins. Plates were incubated at 22° C. under 24-hour light (120-130 μE m⁻² s⁻¹) in a growth chamber. Evaluation of germination and seedling vigor was performed 5 days after planting. For assessment of root development, seedlings germinated on 80% MS+Vitamins+1% sucrose were transferred to square plates at 7 days. Evaluation was done 5 days after transfer following growth in a vertical position. Qualitative differences were recorded including lateral and primary root length, root hair number and length, and overall growth.

For chilling (8° C.) and heat sensitivity (32° C.) growth assays, seeds were germinated and grown for 7 days on MS+Vitamins+1% sucrose at 22° C. and then were transferred to chilling or heat stress conditions. Heat stress was applied for 5 days, after which the plants were transferred back to 22° C. for recovery and evaluated after a further 5 days. Plants were subjected to chilling conditions (8° C.) and evaluated at 10 days and 17 days.

For plate-based severe dehydration assays (sometimes referred to as desiccation assays), seedlings were grown for 14 days on MS+ Vitamins+1% Sucrose at 22° C. Plates were opened in the sterile hood for 3 hr for hardening and then seedlings were removed from the media and dried for 2 h in the hood. After this time they were transferred back to plates and incubated at 22° C. for recovery. Plants were evaluated after another 5 days.

### Data Interpretation

At the time of evaluation, plants were given one of the following scores:
(++) Substantially enhanced performance compared to controls. The phenotype was very consistent and growth was significantly above the normal levels of variability observed for that assay.
(+) Enhanced performance compared to controls. The response was consistent but was only moderately above the normal levels of variability observed for that assay.
(wt) No detectable difference from wild-type controls.
(−) Impaired performance compared to controls. The response was consistent but was only moderately above the normal levels of variability observed for that assay.
(−−) Substantially impaired performance compared to controls. The phenotype was consistent and growth was significantly above the normal levels of variability observed for that assay.
(n/d) Experiment failed, data not obtained, or assay not performed.

### Example VII

### Soil Drought (Clay Pot)

The soil drought assay (performed in clay pots) was based on that described by Haake et al. (2002). Experimental Procedure.

Previously, we performed clay-pot assays on segregating T2 populations, sown directly to soil. However, in the current procedure, seedlings were first germinated on selection plates containing either kanamycin or sulfonamide.

Seeds were sterilized by a 2 minute ethanol treatment followed by 20 minutes in 30% bleach/0.01% Tween and five washes in distilled water. Seeds were sown to MS agar in 0.1% agarose and stratified for 3 days at 4° C., before transfer to growth cabinets with a temperature of 22° C. After 7 days of growth on selection plates, seedlings were transplanted to 3.5 inch diameter clay pots containing 80 g of a 50:50 mix of vermiculite:perlite topped with 80 g of ProMix. Typically, each pot contains 14 seedlings, and plants of the transgenic line being tested are in separate pots to the wild-type controls. Pots containing the transgenic line versus control pots were interspersed in the growth room, maintained under 24-hour light conditions (18-23° C., and 90-100 μE m⁻² s⁻¹) and watered for a period of 14 days. Water was then withheld and pots were placed on absorbent paper for a period of 8-10 days to apply a drought treatment. After this period, a visual qualitative “drought score” from 0-6 was assigned to record the extent of visible drought stress symptoms. A score of “6” corresponded to no visible symptoms whereas a score of “0” corresponded to extreme wilting and the leaves having a “crispy” texture. At the end of the drought period, pots were re-watered and scored after 5-6 days; the number of surviving plants in each pot was counted, and the proportion of the total plants in the pot that survived was calculated.

Split-pot method. A variation of the above method was sometimes used, whereby plants for a given transgenic line were compared to wild-type controls in the same pot. For those studies, 7 wild-type seedlings were transplanted into one half of a 3.5 inch pot and 7 seedlings of the line being tested were transplanted into the other half of the pot.

Analysis of results. In a given experiment, we typically compared 6 or more pots of a transgenic line with 6 or more pots of the appropriate control. (In the split pot method, 12 or more pots are used.) The mean drought score and mean proportion of plants surviving (survival rate) were calculated for both the transgenic line and the wild-type pots. In each case a p-value* was calculated, which indicated the significance of the difference between the two mean values. The results for each transgenic line across each planting for a particular project were then presented in a results table.

Calculation of p-values. For the assays where control and experimental plants were in separate pots, survival was analyzed with a logistic regression to account for the fact that the random variable was a proportion between 0 and 1. The reported p-value was the significance of the experimental proportion contrasted to the control, based upon regressing the logit-transformed data.

Drought score, being an ordered factor with no real numeric meaning, was analyzed with a non-parametric test between the experimental and control groups. The p-value was calculated with a Mann-Whitney rank-sum test.

For the split-pot assays, matched control and experimental measurements were available for both variables. In lieu of a direct transformed regression technique for these data, the logit-transformed proportions were analyzed by parametric methods. The p-value was derived from a paired-t-test on the transformed data. For the paired score data, the p-value from a Wilcoxon test was reported.

### Example IX

### Soil Drought (Single Pot)

These experiments determined the physiological basis for the drought tolerance conferred by each lead and were typically performed under soil grown conditions. Usually, the experiment was performed under photoperiodic conditions of 10-hr or 12-hr light. Where possible, a given project (gene/promoter combination or protein variant) was represented by three independent lines. Plants were usually at late vegetative/early reproductive stage at the time measurements were taken. Typically we assayed three different states: a well-watered state, a mild-drought state and a moderately severe drought state. In each case, we made comparisons to wild-type plants with the same degree of physical stress symptoms (wilting). To achieve this, staggered samplings were often required. Typically, for a given line, ten individual plants were assayed for each state.

The following physiological parameters were routinely measured: relative water content, ABA content, proline content, and photosynthesis rate. In some cases, measurements of chlorophyll levels, starch levels, carotenoid levels, and chlorophyll fluorescence were also made.

Analysis of results. In a given experiment, for a particular parameter, we typically compared about 10 samples from a given transgenic line with about 10 samples of the appropriate wild-type control at each drought state. The mean values for each physiological parameter were calculated for both the transgenic line and the wild-type pots. In each case, a P-value (calculated via a simple t-test) was determined, which indicated the significance of the difference between the two mean values. The results for each transgenic line across each planting for a particular project were then presented in a results table.

A typical procedure is described below; this corresponds to method used for the drought time-course experiment which we performed on wild-type plants during our baseline studies at the outset of the drought program.

Procedure. Seeds were stratified for 3 days at 4° C. in 0.1% agarose and sown on Metronmix 200 in 2.25 inch pots (square or round). Plants were maintained in individual pots within flats grown under short days (10:14 L:D). Seeded were watered as needed to maintain healthy plant growth and development. At 7 to 8 weeks after planting, plants were used in drought experiments.

Plants matched for equivalent growth development (rosette size) were removed from plastic flats and placed on absorbent paper. Pots containing plants used as well-watered controls were placed within a weigh boat and the dish placed on the absorbent paper. The purpose of the weigh boat was to retain any water that might leak from well-watered pots and affect pots containing plants undergoing the drought stress treatment.

On each day of sampling, up to 18 droughted plants and 6 well-watered controls (from each transgenic line) were picked from a randomly generated pool (given that they passed quality control standards). Biochemical analysis for photosynthesis, ABA, and proline was performed on the next three youngest, most fully expanded leaves. Relative water content was analyzed using the remaining rosette tissue.

### Example X

### Soil Drought (Biochemical and Physiological Assays)

Background. The purpose of these measurements was to determine the physiological state of plants in soil drought experiments.

Measurement of Photosynthesis. Photosynthesis was measured using a LICOR LI-6400. The LI-6400 uses infrared gas analyzers to measure carbon dioxide to generate a photosynthesis measurement. This method is based upon the difference of the CO₂ reference (the amount put into the chamber) and the CO₂ sample (the amount that leaves the chamber). Since photosynthesis is the process of converting CO₂ to carbohydrates, we expected to see a decrease in the amount of CO₂ sample. From this difference, a photosynthesis rate can be generated. In some cases, respiration may occur and an increase in CO₂ detected. To perform measurements, the L1-6400 was set-up and calibrated as per L1-6400 standard directions. Photosynthesis was measured in the youngest most fully expanded leaf at 300 and 1000 ppm CO₂ using a metal halide light source. This light source provided about 700 μE m⁻² s⁻¹.

Fluorescence was measured in dark and light adapted leaves using either a L1-6400 (LICOR) with a leaf chamber fluorometer attachment or an OS-1 (Opti-Sciences) as described in the manufacturer's literature. When the LI-6400 was used, all manipulations were performed under a dark shade cloth. Plants were dark adapted by placing in a box under this shade cloth until used. The OS-30 utilized small clips to create dark adapted leaves.

Measurement of Abscisic Acid and Proline. The purpose of this experiment was to measure ABA and proline in plant tissue. ABA is a plant hormone believed to be involved in stress responses and proline is an osmoprotectant.

Three of the youngest, most fully expanded mature leaves were harvested, frozen in liquid nitrogen, lyophilized, and a dry weight measurement taken. Plant tissue was then homogenized in methanol to which 500 ng of d6-ABA had been added to act as an internal standard. The homogenate was filtered to removed plant material and the filtrate evaporated to a small volume. To this crude extract, approximately 3 ml of 1% acetic acid was added and the extract was further evaporated to remove any remaining methanol. The volume of the remaining aqueous extract was measured and a small aliquot (usually 200 to 500 μl) removed for proline analysis (Protocol described below). The remaining extract was then partitioned twice against ether, the ether removed by evaporation and the residue methylated using ethereal diazomethane. Following removal of any unreacted diazomethane, the residue was dissolved in 100 to 200 μl ethyl acetate and analyzed by gas chromatography-mass spectrometry. Analysis was performed using an HP 6890 GC coupled to an HP 5973 MSD using a DB-5 ms gas capillary column. Column pressure was 20 psi. Initially, the oven temperature was 150° C. Following injection, the oven was heated at 5° C./min to a final temperature of 250° C. ABA levels were estimated using an isotope dilution equation and normalized to tissue dry weight.

Free proline content was measured according to Bates (Bates et al., 1973). The crude aqueous extract obtained above was brought up to a final volume of 500 μl using distilled water. Subsequently, 500 μl of glacial acetic was added followed by 500 μl of Chinard's Ninhydrin. The samples were then heated at 95 to 100° C. for 1 hour. After this incubation period, samples were cooled and 1.5 ml of toluene were added. The upper toluene phase was removed and absorbance measured at 515 nm. Amounts of proline were estimated using a standard curve generated using L-proline and normalized to tissue dry weight.

[n.b. Chinard's Ninhydrin was prepared by dissolving 2.5 g ninhydrin (triketohydrindene hydrate) in 60 ml glacial acetic acid at 70° C. to which 40 ml of 6 M phosphoric acid was added.]

Measurement of Relative Water Content (RWC). Relative Water Content (RWC) indicates the amount of water that is stored within the plant tissue at any given time. It was obtained by taking the field weight of the rosette minus the dry weight of the plant material and dividing by the weight of the rosette saturated with water minus the dry weight of the plant material. The resulting RWC value can be compared from plant to plant, regardless of plant size.

$$Relative Water Content = \frac{Field Weight - Dry Weight}{Turgid Weight - Dry Weight} \times 100$$

After tissue had been removed for array and ABA/proline analysis, the rosette was cut from the roots using a small pair of scissors. The field weight was obtained by weighing the rosette. The rosette was then immersed in cold water and placed in an ice water bath in the dark. The purpose of this was to allow the plant tissue to take up water while preventing any metabolism which could alter the level of small molecules within the cell. The next day, the rosette was carefully removed, blotted dry with tissue paper, and weighed to obtain the turgid weight. Tissue was then frozen, lyophilized, and weighed to obtain the dry weight.

Starch determination. Starch was estimated using a simple iodine based staining procedure. Young, fully expanded leaves were harvested either at the end or beginning of a 12 h light period and placed in tubes containing 80% ethanol or 100% methanol. Leaves were decolorized by incubating tubes in a 70 to 80 C water bath until chlorophyll had been removed from leaf tissue. Leaves were then immersed in water to displace any residual methanol which may be present in the tissue. Starch was then stained by incubating leaves in an iodine stain (2 g KI, 1 g I₂ in 100 ml water) for one min and then washing with copious amounts of water. Tissue containing large amounts of starch stained dark blue or black; tissues depleted in starch were colorless.

Chlorophyll/carotenoid determination. For some experiments, chlorophyll was estimated in methanolic extracts using the method of Porra et al. (1989). Carotenoids were estimated in the same extract at 450 nm using an A (1%) of 2500. We currently are measuring chlorophyll using a SPAD-502 (Minolta). When the SPAD-502 was being used to measure chlorophyll, both carotenoid and chlorophyll content and amount could also be determined via HPLC. Pigments were extracted from leave tissue by homogenizing leaves in acetone:ethyl acetate (3:2). Water was added, the mixture centrifuged, and the upper phase removed for HPLC analysis. Samples were analyzed using a Zorbax C18 (non-endcapped) column (250×4.6) with a gradient of acetonitrile:water (85:15) to acetonitrile:methanol (85:15) in 12.5 minutes. After holding at these conditions for two minutes, solvent conditions were changed to methanol:ethyl acetate (68:32) in two minutes.

Carotenoids and chlorophylls were quantified using peak areas and response factors calculated using lutein and beta-carotene as standards.

Quantification of protein level. Protein level quantification was performed for 35S::G481 and related projects. Plants were plated on selective MS media, and transplanted to vertical MS plates after one week of growth. After 17 days of growth (24 h light, 22 C), tissues were harvested from the vertical plates. The shoot tissue from 1 plant was harvested as one biological replicate for each line, and the root tissue from 2 plants were combined as 1 biological replicate. For each line analyzed, two biological replicates each of shoot and root tissue were analyzed. Whole cell protein extracts were prepared in a 96 well format and separated on a 4-20% SDS-PAGE gel, transferred to PVDF membrane for western blotting, and probed with a 1:2000 dilution of anti-G481 antibody in a 1% blocking solution in TBS-T. Protein levels for various samples were estimated by setting a level of one for pMEN65 wild type and three for line G481-6 to describe the amount of G481 protein visible on the blot. The protein level for each of the other lines tested was visually estimated on each blot relative to the pMEN65 and G481-6 standards.

Nuclear and cytoplasmically-enriched fractions. We developed a platform to prepare nuclear and cytoplasmic protein extracts in a 96-well format using a tungsten carbide beads for cell disruption in a mild detergent and a sucrose cushion to separate cytoplasmic from nuclear fractions. We used histone antibodies to demonstrate that this method effectively separated cytoplasmic from nuclear-enriched fractions. An alternate method (spun only) used the same disruption procedure, but simply pelleted the nuclei to separate them from the cytoplasm without the added purification of a sucrose cushion.

Quantification of mRNA level. Three shoot and three root biological replicates were typically harvested for each line, as described above in the protein quantification methods section. RNA was prepared using a 96-well format protocol, and cDNA synthesized from each sample. These preparations were used as templates for RT-PCR experiments. We measured the levels of transcript for a gene of interest (such as G481) relative to 18S RNA transcript for each sample using an ABI 7900 Real-Time RT-PCR machine with SYBR Green technology.

Phenotypic Analysis: Flowering time. Plants were grown in soil. Flowering time was determined based on either or both of (i) number to days after planting to the first visible flower bud. (ii) the total number of leaves (rosette or rosette plus cauline) produced by the primary shoot meristem.

Phenotypic Analysis: Heat stress. In preliminary experiments described in this report, plants were germinated growth chamber at 30 C with 24 h light for 11 d. Plants were allowed to recover in 22 C with 24 h light for three days, and photographs were taken to record health after the treatment. In a second experiment, seedlings were grown at 22 C for four days on selective media, and the plates transferred to 32 C for one week. They were then allowed to recover at 22 C for three days. Forty plants from two separate plates were harvested for each line, and both fresh weight and chlorophyll content measured.

Phenotypic Analysis: Dark-induced senescence. In preliminary experiments described in this report, plants were grown on soil for 27-30 days in 12 h light at 22 C. They were moved to a dark chamber at 22 C, and visually evaluated for senescence after 10-13 days. In some cases we used Fv/Fm as a measure of chlorophyll (Pourtau et al., 2004) on the youngest most fully-expanded leaf on each plant. The Fv/Fm mean for the 12 plants from each line was normalized to the Fv/Fm mean for the 12 matched controls.

Microscopy. Light microscopy was performed by us. Electron and confocal microscopy were performed using the facilities at University of California, Berkeley.

Various Definitions Used in this Report:
RWC=Relative water content (field wt.−dry weight)/(turgid wt.−dry wt.)×100
ABA=Abscisic acid, μg/gdw
Proline=Proline, μmole/gdw
A 300=net assimilation rate, μmole CO₂/m²/s at 300 ppm CO₂
A 1000=net assimilation rate, μmole CO₂/m²/s at 1000 ppm CO₂
Ch1 SPAD=Chlorophyll estimated by a Minolta SPAD-502, ratio of 650 nm to 940 nm
Total Ch1=mg/gfw, estimated by HPLC
Carot=mg/gfw, estimated by HPLC
Fo=minimal fluorescence of a dark adapted leaf
Fm′=maximal fluorescence of a dark adapted leaf
Fo′=minimal fluorescence of a light adapted leaf
Fm′=maximal fluorescence of a light adapted leaf
Fs=steady state fluorescence of a light adapted leaf
Psi lf=water potential (Mpa) of a leaf
Psi p=turgor potential (Mpa) of a leaf
Psi pi=osmotic potential (Mpa) of a leaf
Fv/Fm=(Fm−Fo)/Fm; maximum quantum yield of PSII
Fv′/Fm′=(Fm′−Fo′)/Fm′; efficiency of energy harvesting by open PSII reaction centers
PhiPS2=(Fm′−Fs)/Fm′, actual quantum yield of PSII
ETR=PhiPS2×light intensity absorbed×0.5; we use 100 μE/m²/s for an average light intensity and 85% as the amount of light absorbed
qP=(Fm′−Fs)/(Fm′−Fo′); photochemical quenching (includes photosynthesis and photorespiration); proportion of open PSII
qN=(Fm−Fm′)/(Fm−Fo′); non-photochemical quenching (includes mechanisms like heat dissipation)
NPQ=(Fm−Fm′)/Fm′; non-photochemical quenching (includes mechanisms like heat dissipation)

### Example XI

### Disease Physiology, Plate Assays

Overview. A Sclerotinia plate-based assay was used as a pre-screen to identify top performing lines from each project (i.e., lines from transformation with a particular construct) that could be tested in subsequent soil-based assays. Top performing lines were also subjected to Botrytis cinerea plate assays as noted. Typically, eight lines were subjected to plate assays, from which the best lines were selected for subsequent soil-based assays. In projects where significant pathogen resistance was not obtained in plate based assays, lines were not submitted for soil assays.

Unless otherwise stated, all experiments were performed with the Arabidopsis thaliana ecotype Columbia (Col-0). Assays were usually performed on non-selected segregating T2 populations (in order to avoid the extra stress of selection). Control plants for assays on lines containing direct promoter-fusion constructs were wild-type plants or Col-0 plants transformed an empty transformation vector (pMEN65). Controls for 2-component lines (generated by supertransformation) were the background promoter-driver lines (i.e. promoter::LexA-GAL4TA lines), into which the supertransformations were initially performed.

Procedures. Prior to plating, seed for all experiments were surface sterilized in the following manner: (1) 5 minute incubation with mixing in 70% ethanol; (2) 20 minute incubation with mixing in 30% bleach, 0.01% Triton X-100; (3) five rinses with sterile water. Seeds were resuspended in 0.1% sterile agarose and stratified at 4° C. for 2-4 days.

Sterile seeds were sown on starter plates (15 mm deep) containing the following medium: 50% MS solution, 1% sucrose, 0.05% MES, and 1% Bacto-Agar. 40 to 50 seeds were sown on each plate. Plates were incubated at 22° C. under 24-hour light (95-110 μE m² s⁻¹) in a germination growth chamber. On day 10, seedlings were transferred to assay plates (25 mm deep plates with medium minus sucrose). Each assay plate had nine test seedlings and nine control seedlings on separate halves of the plate. Three or four plates were used per line, per pathogen. On day 14, seedlings were inoculated (specific methods below). After inoculation, plates were put in a growth chamber under a 12-hour light/12-hour dark schedule. Light intensity was lowered to 70-80 μE m² s⁻¹ for the disease assay. Disease symptoms were evaluated starting four days post-inoculation (DPI) up to 10 DPI if necessary. For each plate, the number of dead test plants and control plants were counted. Plants were scored as “dead” if the center of the rosette collapsed (usually brown or water-soaked).

Sclerotinia inoculum preparation. A Sclerotinia liquid culture was started three days prior to plant inoculation by cutting a small agar plug (¼ sq. inch) from a 14- to 21-day old Sclerotinia plate (on Potato Dextrose Agar; PDA) and placing it into 100 ml of half-strength Potato Dextrose Broth (PDB). The culture was allowed to grown in the PDB at room temperature under 24-hour light for three days. On the day of seedling inoculation, the hyphal ball was retrieved from the medium, weighed, and ground in a blender with water (50 ml/gm tissue). After grinding, the mycelial suspension was filtered through two layers of cheesecloth and the resulting suspension was diluted 1:5 in water. Plants were inoculated by spraying to run-off with the mycelial suspension using a Preval aerosol sprayer.

Botrytis inoculum preparation. Botrytis inoculum was prepared on the day of inoculation. Spores from a 14- to 21-day old plate were resuspended in a solution of 0.05% glucose, 0.03M KH₂PO₄ to a final concentration of 10⁴ spores/ml. Seedlings were inoculated with a Preval aerosol sprayer, as with Sclerotinia inoculation.

Data Interpretation. After the plates were evaluated, each line was given one of the following overall scores:

(++) Substantially enhanced resistance compared to controls. The phenotype was very consistent across all plates for a given line.

(+) Enhanced resistance compared to controls. The response was consistent but was only moderately above the normal levels of variability observed for that assay.

(wt) No detectable difference from wild-type controls.

(−) Increased susceptibility compared to controls. The response was consistent but was only moderately above the normal levels of variability observed for that assay.

(−−) Substantially impaired performance compared to controls. The phenotype was consistent and growth was significantly above the normal levels of variability observed for that assay.

(n/d) Experiment failed, data not obtained, or assay not performed.

### Example XII

### Disease Physiology, Soil Assays

Overview. Lines from transformation with a particular construct were tested in a soil-based assay for resistance to powdery mildew (Erysiphe cichoracearum) as noted below. Typically, eight lines per project were subjected to the Erysiphe assay.

Unless otherwise stated, all experiments were performed with the Arabidopsis thaliana ecotype Columbia (Col-0). Assays were usually performed on non-selected segregating T2 populations (in order to avoid the extra stress of selection). Control plants for assays on lines containing direct promoter-fusion constructs were wild-type plants or Col-0 plants transformed an empty transformation vector (pMEN65). Controls for 2-component lines (generated by supertransformation) were the background promoter-driver lines (i.e. promoter::LexA-GAL4TA lines), into which the supertransformations were initially performed.

In addition, positive hits from the Sclerotinia plate assay were subjected to a soil-based Sclerotinia assay as noted. This assay was based on hyphal plug inoculation of rosette leaves.

Procedures. Erysiphe inoculum was propagated on a pad4 mutant line in the Col-0 background, which is highly susceptible to Erysiphe (Reuber et al., 1998). The inoculum was maintained by using a small paintbrush to dust conidia from a 2-3 week old culture onto new plants (generally three weeks old). For the assay, seedlings were grown on plates for one week under 24-hour light in a germination chamber, then transplanted to soil and grown in a walk-in growth chamber under a 12-hour light/12-hour dark light regimen, 70% humidity. Each line was transplanted to two 13 cm square pots, nine plants per pot. In addition, three control plants were transplanted to each pot for direct comparison with the test line. Approximately 3.5 weeks after transplanting, plants were inoculated using settling towers as described by Reuber et al. (1998). Generally, three to four heavily infested leaves were used per pot for the disease assay. The level of fungal growth was evaluated eight to ten days after inoculation.

Data Interpretation. After the pots were evaluated, each line was given one of the following overall scores:

(+++) Highly enhanced resistance as compared to controls. The phenotype was very consistent.

(++) Substantially enhanced resistance compared to controls. The phenotype was very consistent in both pots for a given line.

(+) Enhanced resistance compared to controls. The response was consistent but was only moderately above the normal levels of variability observed.

(wt) No detectable difference from wild-type controls.

(−) Increased susceptibility compared to controls. The response was consistent but was only moderately above the normal levels of variability observed.

(−−) Substantially impaired performance compared to controls. The phenotype was consistent and growth was significantly above the normal levels of variability observed.

(n/d) Experiment failed, data not obtained, or assay not performed.

### Example XIII

### Experimental Results

This report provides experimental observations for ten transcription factors for drought tolerance (G481; G682; G867; G912; G1073; G47; G1274; G1792; G2999; G3086) and two transcription factors for disease resistance (G28; G1792). A set of polynucleotides and polypeptides related to each lead transcription factor has been designated as a “study group” and related sequences in these clades have been subsequently analyzed using morphological and phenotypic studies.

Phenotypic Screens: promoter combinations. A panel of promoters was assembled based on domains of expression that had been well characterized in the published literature. These were chosen to represent broad non-constitutive patterns which covered the major organs and tissues of the plant. The following domain-specific promoters were picked, each of which drives expression in a particular tissue or cell-type: ARSK1 (root), RBCS3 (photosynthetic tissue, including leaf tissue), CUT1 (shoot epidermal, guard-cell enhanced), SUC2 (vascular), STM (apical meristem and mature-organ enhanced), AP1 (floral meristem enhanced), AS1 (young organ primordia) and RSI1 (young seedlings, and roots). Also selected was a stress inducible promoter, RD29A, which is able to up-regulate a transgene at drought onset.

The basic strategy was to test each polynucleotide with each promoter to give insight into the following questions: (i) mechanistically, in which part of the plant is activity of the polynucleotide sufficient to produce stress tolerance? (ii) Can we identify expression patterns which produce compelling stress tolerance while eliminating any undesirable effects on growth and development? (iii) Does a particular promoter give an enhanced or equivalent stress tolerance phenotype relative to constitutive expression? Each of the promoters in this panel is considered to be representative of a particular pattern of expression; thus, for example, if a particular promoter such as SUC2, which drives expression in vascular tissue, yields a positive result with a particular transcription factor gene, it would be predicted and expected that a positive result would be obtained with any other promoter that drives the same vascular pattern.

We now have many examples demonstrating the principle that use of a regulated promoter can confer substantial stress tolerance while minimizing deleterious effects. For example, the results from regulating G1792-related genes using regional specific promoters were especially persuasive. When overexpressed constitutively, these genes produced extreme dwarfing. However, when non-constitutive promoters were used to express these sequences ectopically, off-types were substantially ameliorated, and strong disease tolerance was still obtained (for example, with RBCS3::G1792 and RBCS3::G1795 lines). Another project worth highlighting is ARSK1::G867 where expression in the roots yielded drought tolerance without any apparent off-types.

Additionally, it is feasible to identify promoters which afford high levels of inducible expression. For instance, a major tactic in the disease program is to utilize pathogen inducible promoters; a set of these has now been identified for testing with each of the disease-resistance conferring transcription factors. This approach is expected to be productive as we have shown that inducible expression of G1792 via the dexamethasone system gives effective disease tolerance without off-types. By analogy, it would be useful to take a similar approach for the drought tolerance trait. So far the only drought regulated promoter that we have tested is RD29A, since its utility had been published (Kasuga et al., 1999).

Phenotypic Screens: effects of protein variants for distinct transcription factors. The effects of overexpressing a variety of different types of protein variants including: deletion variants, GAL4 fusions, variants with specific residues mutagenized, and forms in which domains are swapped with other proteins, have been examined. Together, these approaches have been informative, and have helped illuminate the role of specific residues (see for example, the site-directed mutagenesis experiments for G1274 or G1792), as well as giving new clues as to the basis of particular phenotypes. For example, overexpression lines for a G481 deletion variant exhibited drought tolerance, suggesting that the G481 drought phenotype might arise from dominant negative type interactions.

Phenotypic Screens: knockout and knock-down approaches. Thus far, both T-DNA alleles and RNAi methods have been used to isolate knockouts/knockdown lines for transcription factors of interest. In general, it was determined that the knockout (KO) approach to be more informative and easier to interpret than RNAi based strategies. In particular, RNAi approaches are hampered by the possibility that other related transcription factors might be directly or indirectly knocked-down (even when using a putative gene-specific construct). Thus, a set of RNAi lines showing an interesting phenotype requires a very substantial amount of molecular characterization to prove that the phenotypes are due to reduced activity of the targeted gene. We have found that KO lines have given some useful insights into the relative endogenous roles of particular genes within the CAAT family, and revealed the potential for obtaining stress tolerance traits via knock-down strategies (e.g., G481 knockout/knockdown approaches).

The following table summarizes the experimental results that have yielded new phenotypic traits in morphological, physiological or disease assays in Arabidopsis. The last column lists the trait that was experimentally observed in plants after: (i) transforming with each transcription factor polynucleotide GID (Gene IDentifier, found in the first column) under the listed regulatory control mechanism (found in the fifth or “Project Column”); (ii) in the cases where the project is listed as “KO”, where the transcription factor was knocked out; or (iii) in the cases where the project is listed as “RNAi (GS) or RNAi(clade), the transcription factor was knocked down using RNAi targeting either the gene sequence or the clade of related genes, respectively.

TABLE 25
Phenotypic traits conferred by Arabidopsis transcription factors in morphological, physiological or
disease assays in Arabidopsis

| | | Species from | | | | |
|---|---|---|---|---|---|---|
| | SEQ | which GID | | | | |
| | ID | was | | | | Experimental observation |
| GID | NO: | obtained | Clade | Project | Trait Category | (trait compared to controls) |
| G1006 | 152 | Arabidopsis | G28 | Constitutive | Resistance to | Increased resistance to |
| | | thaliana | | 35S | Sclerotinia | Sclerotinia |
| G3430 | 168 | Oryza sativa | G28 | Constitutive | Resistance to | Increased resistance to |
| | | | | 35S | Sclerotinia | Sclerotinia |
| G3660 | 158 | Brassica | G28 | Constitutive | Resistance to | Increased resistance to |
| | | oleracea | | 35S | Sclerotinia | Sclerotinia |
| G3718 | 156 | Glycine max | G28 | Constitutive | Resistance to | Increased resistance to |
| | | | | 35S | Sclerotinia | Sclerotinia |
| G3717 | 154 | Glycine max | G28 | Constitutive | Resistance to | Increased resistance to |
| | | | | 35S | Erysiphe | Erysiphe |
| G3659 | 150 | Brassica | G28 | Constitutive | Resistance to | Increased resistance to |
| | | oleracea | | 35S | Erysiphe | Erysiphe |
| G3718 | 156 | Glycine max | G28 | Constitutive | Resistance to | Increased resistance to |
| | | | | 35S | Erysiphe | Erysiphe |
| G2133 | 176 | Arabidopsis | G47 | Constitutive | Altered | Inflorescence: decreased |
| | | thaliana | | 35S | architecture | apical dominance |
| G47 | 174 | Arabidopsis | G47 | Leaf RBCS3 | Cold tolerance | Increased tolerance to cold |
| | | thaliana | | | | |
| G2115 | 406 | Arabidopsis | G47 | Constitutive | Cold tolerance | Increased tolerance to cold |
| | | thaliana | | 35S | | |
| G2133 | 176 | Arabidopsis | G47 | Constitutive | Cold tolerance | Increased tolerance to cold |
| | | thaliana | | 35S | | |
| G3643 | 178 | Glycine max | G47 | Constitutive | Cold tolerance | Increased tolerance to cold |
| | | | | 35S | | |
| G3649 | 184 | Oryza sativa | G47 | Constitutive | Cold tolerance | Increased tolerance to cold |
| | | | | 35S | | |
| G47 | 174 | Arabidopsis | G47 | Stress | Altered | Decreased ABA sensitivity |
| | | thaliana | | Inducible | hormone | |
| | | | | RD29A | sensitivity | |
| G47 | 174 | Arabidopsis | G47 | Stress | Drought | Increased tolerance to |
| | | thaliana | | Inducible | tolerance | dehydration |
| | | | | RD29A | | |
| G2133 | 176 | Arabidopsis | G47 | Constitutive | Drought | Increased tolerance to |
| | | thaliana | | 35S | tolerance | dehydration |
| G2133 | 176 | Arabidopsis | G47 | Leaf RBCS3 | Drought | Increased tolerance to |
| | | thaliana | | | tolerance | dehydration |
| G2133 | 176 | Arabidopsis | G47 | Stress | Drought | Increased tolerance to |
| | | thaliana | | Inducible | tolerance | dehydration |
| | | | | RD29A | | |
| G3643 | 178 | Glycine max | G47 | Constitutive | Drought | Increased tolerance to drought |
| | | | | 35S | tolerance | in soil assays |
| G47 | 174 | Arabidopsis | G47 | GAL4 N- | Altered | Early flowering |
| | | thaliana | | term (Super | flowering time | |
| | | | | Active) | | |
| G47 | 174 | Arabidopsis | G47 | Vascular | Altered | Late flowering |
| | | thaliana | | SUC2 | flowering time | |
| G3649 | 184 | Oryza sativa | G47 | Constitutive | Altered | Late flowering |
| | | | | 35S | flowering time | |
| G47 | 174 | Arabidopsis | G47 | Shoot apical | Altered leaf | Large leaf size |
| | | thaliana | | meristem | morphology | |
| | | | | STM | | |
| G47 | 174 | Arabidopsis | G47 | Vascular | Altered leaf | Dark green leaf color |
| | | thaliana | | SUC2 | morphology | |
| G47 | 174 | Arabidopsis | G47 | Vascular | Altered leaf | Large leaf size |
| | | thaliana | | SUC2 | morphology | |
| G47 | 174 | Arabidopsis | G47 | Vascular | Altered stem | Thicker stem |
| | | thaliana | | SUC2 | morphology | |
| G3644 | 182 | Oryza sativa | G47 | Constitutive | Altered stem | Thicker stem |
| | | | | 35S | morphology | |
| G3649 | 184 | Oryza sativa | G47 | Constitutive | Altered stem | Thicker stem |
| | | | | 35S | morphology | |
| G481 | 22 | Arabidopsis | G481 | Constitutive | Altered | Increased chlorophyll |
| | | thaliana | | 35S | biochemistry | |
| G481 | 2 | Arabidopsis | G481 | Constitutive | Altered | Increased starch |
| | | thaliana | | 35S | biochemistry | |
| G481 | 2 | Arabidopsis | G481 | Constitutive | Altered | Photosynthesis rate increased |
| | | thaliana | | 35S | biochemistry | |
| G481 | 2 | Arabidopsis | G481 | Vascular | Cold tolerance | Increased tolerance to cold |
| | | thaliana | | SUC2 | | |
| G481 | 2 | Arabidopsis | G481 | Constitutive | Cold tolerance | Increased tolerance to cold |
| | | thaliana | | 35S | | |
| G481 | 2 | Arabidopsis | G481 | RNAi (GS) | Cold tolerance | Increased tolerance to cold |
| | | thaliana | | | | |
| G485 | 18 | Arabidopsis | G481 | Constitutive | Cold tolerance | Increased tolerance to cold |
| | | thaliana | | 35S | | |
| G489 | 46 | Arabidopsis | G481 | Constitutive | Cold tolerance | Increased tolerance to cold |
| | | thaliana | | 35S | | |
| G926 | 52 | Arabidopsis | G481 | KO | Cold tolerance | Increased tolerance to cold |
| | | thaliana | | | | |
| G928 | 400 | Arabidopsis | G481 | Constitutive | Cold tolerance | Increased tolerance to cold |
| | | thaliana | | 35S | | |
| G1248 | 360 | Arabidopsis | G481 | Constitutive | Cold tolerance | Increased tolerance to cold |
| | | thaliana | | 35S | | |
| G1820 | 44 | Arabidopsis | G481 | Constitutive | Cold tolerance | Increased tolerance to cold |
| | | thaliana | | 35S | | |
| G1836 | 48 | Arabidopsis | G481 | Constitutive | Cold tolerance | Increased tolerance to cold |
| | | thaliana | | 35S | | |
| G2345 | 22 | Arabidopsis | G481 | Constitutive | Cold tolerance | Increased tolerance to cold |
| | | thaliana | | 35S | | |
| G2539 | 408 | Arabidopsis | G481 | Constitutive | Cold tolerance | Increased tolerance to cold |
| | | thaliana | | 35S | | |
| G3396 | 42 | Oryza sativa | G481 | Constitutive | Cold tolerance | Increased tolerance to cold |
| | | | | 35S | | |
| G3397 | 36 | Oryza sativa | G481 | Constitutive | Cold tolerance | Increased tolerance to cold |
| | | | | 35S | | |
| G3398 | 40 | Oryza sativa | G481 | Constitutive | Cold tolerance | Increased tolerance to cold |
| | | | | 35S | | |
| G3475 | 16 | Glycine max | G481 | Constitutive | Cold tolerance | Increased tolerance to cold |
| | | | | 35S | | |
| G3476 | 20 | Glycine max | G481 | Constitutive | Cold tolerance | Increased tolerance to cold |
| | | | | 35S | | |
| G3876 | 8 | Oryza sativa | G481 | Constitutive | Cold tolerance | Increased tolerance to cold |
| | | | | 35S | | |
| G481 | 2 | Arabidopsis | G481 | Deletion | Drought | Increased tolerance to drought |
| | | thaliana | | variant | tolerance | in soil assays |
| G481 | 2 | Arabidopsis | G481 | RNAi (GS) | Drought | Increased tolerance to drought |
| | | thaliana | | | tolerance | in soil assays |
| G481 | 2 | Arabidopsis | G481 | Vascular | Drought | Increased tolerance to |
| | | thaliana | | SUC2 | tolerance | dehydration |
| G481 | 2 | Arabidopsis | G481 | Vascular | Drought | Increased tolerance to drought |
| | | thaliana | | SUC2 | tolerance | in soil assays |
| G482 | 28 | Arabidopsis | G481 | Constitutive | Drought | Increased tolerance to drought |
| | | thaliana | | 35S | tolerance | in soil assays |
| G485 | 18 | Arabidopsis | G481 | Constitutive | Drought | Increased tolerance to drought |
| | | thaliana | | 35S | tolerance | in soil assays |
| G485 | 18 | Arabidopsis | G481 | KO | Drought | Increased tolerance to drought |
| | | thaliana | | | tolerance | in soil assays |
| G634 | 50 | Arabidopsis | G481 | Constitutive | Drought | Increased tolerance to |
| | | thaliana | | 35S | tolerance | dehydration |
| G1248 | 360 | Arabidopsis | G481 | Constitutive | Drought | Increased tolerance to drought |
| | | thaliana | | 35S | tolerance | in soil assays |
| G1818 | 404 | Arabidopsis | G481 | Constitutive | Drought | Increased tolerance to |
| | | thaliana | | 35S | tolerance | dehydration |
| G1820 | 44 | Arabidopsis | G481 | Constitutive | Drought | Increased tolerance to drought |
| | | thaliana | | 35S | tolerance | in soil assays |
| G1836 | 48 | Arabidopsis | G481 | Constitutive | Drought | Increased tolerance to drought |
| | | thaliana | | 35S | tolerance | in soil assays |
| G2345 | 22 | Arabidopsis | G481 | Constitutive | Drought | Increased tolerance to drought |
| | | thaliana | | 35S | tolerance | in soil assays |
| G2539 | 408 | Arabidopsis | G481 | Constitutive | Drought | Increased tolerance to drought |
| | | thaliana | | 35S | tolerance | in soil assays |
| G3074 | 410 | Arabidopsis | G481 | Constitutive | Drought | Increased tolerance to |
| | | thaliana | | 35S | tolerance | dehydration |
| G3395 | 38 | Oryza sativa | G481 | Constitutive | Drought | Increased tolerance to drought |
| | | | | 35S | tolerance | in soil assays |
| G3398 | 40 | Oryza sativa | G481 | Constitutive | Drought | Increased tolerance to drought |
| | | | | 35S | tolerance | in soil assays |
| G3434 | 12 | Zea mays | G481 | Constitutive | Drought | Increased tolerance to |
| | | | | 35S | tolerance | dehydration |
| G3435 | 30 | Zea mays | G481 | Constitutive | Drought | Increased tolerance to drought |
| | | | | 35S | tolerance | in soil assays |
| G3470 | 4 | Glycine max | G481 | Constitutive | Drought | Increased tolerance to drought |
| | | | | 35S | tolerance | in soil assays |
| G3471 | 6 | Glycine max | G481 | Constitutive | Drought | Increased tolerance to drought |
| | | | | 35S | tolerance | in soil assays |
| G3476 | 20 | Glycine max | G481 | Constitutive | Drought | Increased tolerance to |
| | | | | 35S | tolerance | dehydration |
| G3476 | 20 | Glycine max | G481 | Constitutive | Drought | Increased tolerance to drought |
| | | | | 35S | tolerance | in soil assays |
| G3876 | 8 | Oryza sativa | G481 | Constitutive | Drought | Increased tolerance to |
| | | | | 35S | tolerance | dehydration |
| G481 | 2 | Arabidopsis | G481 | GAL4 C- | Altered | Early flowering |
| | | thaliana | | term (Super | flowering time | |
| | | | | Active) | | |
| G481 | 2 | Arabidopsis | G481 | RNAi (clade) | Altered | Late flowering |
| | | thaliana | | | flowering time | |
| G481 | 2 | Arabidopsis | G481 | Vascular | Altered | Late flowering |
| | | thaliana | | SUC2 | flowering time | |
| G482 | 28 | Arabidopsis | G481 | Constitutive | Altered | Early flowering |
| | | thaliana | | 35S | flowering time | |
| G482 | 28 | Arabidopsis | G481 | Vascular | Altered | Early flowering |
| | | thaliana | | SUC2 | flowering time | |
| G3397 | 36 | Oryza sativa | G481 | Constitutive | Altered | Early flowering |
| | | | | 35S | flowering time | |
| G3398 | 40 | Oryza sativa | G481 | Constitutive | Altered | Early flowering |
| | | | | 35S | flowering time | |
| G3435 | 30 | Zea mays | G481 | Constitutive | Altered | Early flowering |
| | | | | 35S | flowering time | |
| G3436 | 34 | Zea mays | G481 | Constitutive | Altered | Early flowering |
| | | | | 35S | flowering time | |
| G3474 | 24 | Glycine max | G481 | Constitutive | Altered | Early flowering |
| | | | | 35S | flowering time | |
| G3475 | 16 | Glycine max | G481 | Constitutive | Altered | Early flowering |
| | | | | 35S | flowering time | |
| G481 | 2 | Arabidopsis | G481 | Constitutive | Altered | Late flowering |
| | | thaliana | | 35S | flowering time | |
| G481 | 2 | Arabidopsis | G481 | KO | Altered | Early flowering |
| | | thaliana | | | flowering time | |
| G1334 | 54 | Arabidopsis | G481 | Constitutive | Altered | Early flowering |
| | | thaliana | | 35S | flowering time | |
| G1781 | 56 | Arabidopsis | G481 | Constitutive | Altered | Early flowering |
| | | thaliana | | 35S | flowering time | |
| G3396 | 42 | Oryza sativa | G481 | Constitutive | Altered | Late flowering |
| | | | | 35S | flowering time | |
| G3429 | 58 | Oryza sativa | G481 | Constitutive | Altered | Late flowering |
| | | | | 35S | flowering time | |
| G3434 | 12 | Zea mays | G481 | Constitutive | Altered | Early flowering |
| | | | | 35S | flowering time | |
| G3470 | 4 | Glycine max | G481 | Constitutive | Altered | Late flowering |
| | | | | 35S | flowering time | |
| G3478 | 26 | Glycine max | G481 | Constitutive | Altered | Early flowering |
| | | | | 35S | flowering time | |
| G481 | 2 | Arabidopsis | G481 | GAL4 C- | Heat tolerance | Increased tolerance to heat |
| | | thaliana | | term (Super | | |
| | | | | Active) | | |
| G3436 | 34 | Zea mays | G481 | Constitutive | Heat tolerance | Increased tolerance to heat |
| | | | | 35S | | |
| G485 | 18 | Arabidopsis | G481 | KO | Altered | Decreased ABA sensitivity |
| | | thaliana | | | hormone | |
| | | | | | sensitivity | |
| G481 | 2 | Arabidopsis | G481 | Constitutive | Altered | Decreased ABA sensitivity |
| | | thaliana | | 35S | hormone | |
| | | | | | sensitivity | |
| G485 | 18 | Arabidopsis | G481 | Constitutive | Altered | Decreased ABA sensitivity |
| | | thaliana | | 35S | hormone | |
| | | | | | sensitivity | |
| G1820 | 44 | Arabidopsis | G481 | Constitutive | Altered | Decreased ABA sensitivity |
| | | thaliana | | 35S | hormone | |
| | | | | | sensitivity | |
| G1836 | 48 | Arabidopsis | G481 | Constitutive | Altered | Decreased ABA sensitivity |
| | | thaliana | | 35S | hormone | |
| | | | | | sensitivity | |
| G3396 | 42 | Oryza sativa | G481 | Constitutive | Altered | Decreased ABA sensitivity |
| | | | | 35S | hormone | |
| | | | | | sensitivity | |
| G481 | 2 | Arabidopsis | G481 | Vascular | Altered leaf | Dark green leaf color |
| | | thaliana | | SUC2 | morphology | |
| G481 | 2 | Arabidopsis | G481 | Constitutive | Altered | Increased seedling size |
| | | thaliana | | 35S | morphology | |
| G481 | 2 | Arabidopsis | G481 | GAL4 C- | Altered | Increased seedling size |
| | | thaliana | | term (Super | morphology | |
| | | | | Active) | | |
| G3397 | 36 | Oryza sativa | G481 | Constitutive | Altered | Increased seedling size |
| | | | | 35S | morphology | |
| G482 | 28 | Arabidopsis | G481 | Constitutive | Tolerance to | Increased tolerance to |
| | | thaliana | | 35S | hyperosmotic | mannitol |
| | | | | | stress | |
| G485 | 18 | Arabidopsis | G481 | Constitutive | Tolerance to | Increased tolerance to sucrose |
| | | thaliana | | 35S | hyperosmotic | |
| | | | | | stress | |
| G926 | 52 | Arabidopsis | G481 | KO | Altered sugar | Increased tolerance to sugar |
| | | thaliana | | | sensing | |
| G928 | 400 | Arabidopsis | G481 | Constitutive | Tolerance to | Increased tolerance to sucrose |
| | | thaliana | | 35S | hyperosmotic | |
| | | | | | stress | |
| G1820 | 44 | Arabidopsis | G481 | Constitutive | Tolerance to | Increased tolerance to sucrose |
| | | thaliana | | 35S | hyperosmotic | and mannitol |
| | | | | | stress | |
| G1836 | 48 | Arabidopsis | G481 | Constitutive | Tolerance to | Increased tolerance to sucrose |
| | | thaliana | | 35S | hyperosmotic | |
| | | | | | stress | |
| G3470 | 4 | Glycine max | G481 | Constitutive | Tolerance to | Increased tolerance to sucrose |
| | | | | 35S | hyperosmotic | and mannitol |
| | | | | | stress | |
| G634 | 50 | Arabidopsis | G481 | Constitutive | Altered root | Increased root mass |
| | | thaliana | | 35S | morphology | |
| G3472 | 32 | Glycine max | G481 | Constitutive | Altered root | Increased root hair |
| | | | | 35S | morphology | |
| G3472 | 32 | Glycine max | G481 | Constitutive | Tolerance to | Increased tolerance to NaCl |
| | | | | 35S | sodium | |
| | | | | | chloride | |
| G485 | 18 | Arabidopsis | G481 | KO | Tolerance to | Increased tolerance to NaCl |
| | | thaliana | | | sodium | |
| | | | | | chloride | |
| G481 | 2 | Arabidopsis | G481 | GAL4 C- | Tolerance to | Increased tolerance to NaCl |
| | | thaliana | | term (Super | sodium | |
| | | | | Active) | chloride | |
| G481 | 2 | Arabidopsis | G481 | KO | Tolerance to | Decreased tolerance to NaCl |
| | | thaliana | | | sodium | |
| | | | | | chloride | |
| G485 | 18 | Arabidopsis | G481 | Constitutive | Tolerance to | Increased tolerance to NaCl |
| | | thaliana | | 35S | sodium | |
| | | | | | chloride | |
| G1820 | 44 | Arabidopsis | G481 | Constitutive | Tolerance to | Increased tolerance to NaCl |
| | | thaliana | | 35S | sodium | |
| | | | | | chloride | |
| G3429 | 58 | Oryza sativa | G481 | Constitutive | Tolerance to | Increased tolerance to NaCl |
| | | | | 35S | sodium | |
| | | | | | chloride | |
| G3434 | 12 | Zea mays | G481 | Constitutive | Tolerance to | Increased tolerance to NaCl |
| | | | | 35S | sodium | |
| | | | | | chloride | |
| G3470 | 4 | Glycine max | G481 | Constitutive | Tolerance to | Increased tolerance to NaCl |
| | | | | 35S | sodium | |
| | | | | | chloride | |
| G2718 | 64 | Arabidopsis | G682 | Constitutive | Altered | Decreased anthocyanin |
| | | thaliana | | 35S | biochemistry | |
| G3392 | 72 | Oryza sativa | G682 | Constitutive | Altered | Decreased anthocyanin |
| | | | | 35S | biochemistry | |
| G3393 | 66 | Oryza sativa | G682 | Constitutive | Altered | Decreased anthocyanin |
| | | | | 35S | biochemistry | |
| G3431 | 68 | Zea mays | G682 | Constitutive | Altered | Decreased anthocyanin |
| | | | | 35S | biochemistry | |
| G3444 | 70 | Zea mays | G682 | Constitutive | Altered | Decreased anthocyanin |
| | | | | 35S | biochemistry | |
| G226 | 62 | Arabidopsis | G682 | Root ARSK1 | Cold tolerance | Increased tolerance to cold |
| | | thaliana | | | | |
| G682 | 60 | Arabidopsis | G682 | Epidermal | Cold tolerance | Increased tolerance to cold |
| | | thaliana | | LTP1 | | |
| G682 | 60 | Arabidopsis | G682 | Vascular | Cold tolerance | Increased tolerance to cold |
| | | thaliana | | SUC2 | | |
| G3392 | 72 | Oryza sativa | G682 | Constitutive | Cold tolerance | Increased tolerance to cold |
| | | | | 35S | | |
| G3393 | 66 | Oryza sativa | G682 | Constitutive | Cold tolerance | Increased tolerance to cold |
| | | | | 35S | | |
| G3431 | 68 | Zea mays | G682 | Constitutive | Cold tolerance | Increased tolerance to cold |
| | | | | 35S | | |
| G3448 | 80 | Glycine max | G682 | Constitutive | Cold tolerance | Increased tolerance to cold |
| | | | | 35S | | |
| G3449 | 78 | Glycine max | G682 | Constitutive | Cold tolerance | Increased tolerance to cold |
| | | | | 35S | | |
| G3450 | 74 | Glycine max | G682 | Constitutive | Cold tolerance | Increased tolerance to cold |
| | | | | 35S | | |
| G1816 | 76 | Arabidopsis | G682 | Constitutive | Drought | Increased tolerance to drought |
| | | thaliana | | 35S | tolerance | in soil assays |
| G3450 | 74 | Glycine max | G682 | Constitutive | Drought | Increased tolerance to drought |
| | | | | 35S | tolerance | in soil assays |
| G682 | 60 | Arabidopsis | G682 | GAL4 N- | Drought | Increased tolerance to |
| | | thaliana | | term (Super | tolerance | dehydration |
| | | | | Active) | | |
| G682 | 60 | Arabidopsis | G682 | Vascular | Drought | Increased tolerance to drought |
| | | thaliana | | SUC2 | tolerance | in soil assays |
| G3446 | 82 | Glycine max | G682 | Constitutive | Drought | Increased tolerance to drought |
| | | | | 35S | tolerance | in soil assays |
| G3447 | 86 | Glycine max | G682 | Constitutive | Drought | Increased tolerance to drought |
| | | | | 35S | tolerance | in soil assays |
| G3448 | 80 | Glycine max | G682 | Constitutive | Drought | Increased tolerance to drought |
| | | | | 35S | tolerance | in soil assays |
| G3445 | 84 | Glycine max | G682 | Constitutive | Altered | Late flowering |
| | | | | 35S | flowering time | |
| G682 | 60 | Arabidopsis | G682 | Vascular | Heat tolerance | Increased tolerance to heat |
| | | thaliana | | SUC2 | | |
| G3450 | 74 | Glycine max | G682 | Constitutive | Heat tolerance | Increased tolerance to heat |
| | | | | 35S | | |
| G226 | 62 | Arabidopsis | G682 | Constitutive | Altered | Decreased ABA sensitivity |
| | | thaliana | | 35S | hormone | |
| | | | | | sensitivity | |
| G682 | 60 | Arabidopsis | G682 | Constitutive | Altered | Decreased ABA sensitivity |
| | | thaliana | | 35S | hormone | |
| | | | | | sensitivity | |
| G682 | 60 | Arabidopsis | G682 | RNAi (GS) | Altered | Decreased ABA sensitivity |
| | | thaliana | | | hormone | |
| | | | | | sensitivity | |
| G682 | 60 | Arabidopsis | G682 | RNAi (clade) | Altered | Decreased ABA sensitivity |
| | | thaliana | | | hormone | |
| | | | | | sensitivity | |
| G3445 | 84 | Glycine max | G682 | Constitutive | Altered | Decreased ABA sensitivity |
| | | | | 35S | hormone | |
| | | | | | sensitivity | |
| G682 | 60 | Arabidopsis | G682 | Constitutive | Altered | Increased tolerance to low |
| | | thaliana | | 35S | nutrient uptake | nitrogen conditions |
| G1816 | 76 | Arabidopsis | G682 | Constitutive | Altered | Altered C/N sensing: |
| | | thaliana | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G1816 | 76 | Arabidopsis | G682 | Constitutive | Altered | Increased tolerance to low |
| | | thaliana | | 35S | nutrient uptake | nitrogen conditions |
| G3393 | 66 | Oryza sativa | G682 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G226 | 62 | Arabidopsis | G682 | Constitutive | Altered | Altered C/N sensing: |
| | | thaliana | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G226 | 62 | Arabidopsis | G682 | Constitutive | Altered | Increased tolerance to low |
| | | thaliana | | 35S | nutrient uptake | nitrogen conditions |
| G682 | 60 | Arabidopsis | G682 | GAL4 C- | Altered | Altered C/N sensing: |
| | | thaliana | | term (Super | nutrient uptake | increased tolerance to basal |
| | | | | Active) | | media minus nitrogen plus 3% |
| | | | | | | sucrose and/or basal media |
| | | | | | | minus nitrogen plus 3% |
| | | | | | | sucrose and 1 mM glutamine |
| G682 | 60 | Arabidopsis | G682 | GAL4 C- | Altered | Increased tolerance to low |
| | | thaliana | | term (Super | nutrient uptake | nitrogen conditions |
| | | | | Active) | | |
| G682 | 60 | Arabidopsis | G682 | GAL4 N- | Altered | Increased tolerance to low |
| | | thaliana | | term (Super | nutrient uptake | nitrogen conditions |
| | | | | Active) | | |
| G682 | 60 | Arabidopsis | G682 | Epidermal | Altered | Altered C/N sensing: |
| | | thaliana | | LTP1 | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G682 | 60 | Arabidopsis | G682 | Epidermal | Altered | Increased tolerance to low |
| | | thaliana | | LTP1 | nutrient uptake | nitrogen conditions |
| G1816 | 76 | Arabidopsis | G682 | Epidermal | Altered | Increased tolerance to low |
| | | thaliana | | CUT1 | nutrient uptake | nitrogen conditions |
| G3392 | 72 | Oryza sativa | G682 | Constitutive | Altered | Increased tolerance to low |
| | | | | 35S | nutrient uptake | nitrogen conditions |
| G3392 | 72 | Oryza sativa | G682 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3393 | 66 | Oryza sativa | G682 | Constitutive | Altered | Increased tolerance to low |
| | | | | 35S | nutrient uptake | nitrogen conditions |
| G3431 | 68 | Zea mays | G682 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3431 | 68 | Zea mays | G682 | Constitutive | Altered | Increased tolerance to low |
| | | | | 35S | nutrient uptake | nitrogen conditions |
| G3444 | 70 | Zea mays | G682 | Constitutive | Altered | Increased tolerance to low |
| | | | | 35S | nutrient uptake | nitrogen conditions |
| G3447 | 86 | Glycine max | G682 | Constitutive | Altered | Increased tolerance to low |
| | | | | 35S | nutrient uptake | nitrogen conditions |
| G3448 | 80 | Glycine max | G682 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3448 | 80 | Glycine max | G682 | Constitutive | Altered | Increased tolerance to low |
| | | | | 35S | nutrient uptake | nitrogen conditions |
| G3449 | 78 | Glycine max | G682 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3449 | 78 | Glycine max | G682 | Constitutive | Altered | Increased tolerance to low |
| | | | | 35S | nutrient uptake | nitrogen conditions |
| G3450 | 74 | Glycine max | G682 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3450 | 70 | Glycine max | G682 | Constitutive | Altered | Increased tolerance to low |
| | | | | 35S | nutrient uptake | nitrogen conditions |
| G682 | 60 | Arabidopsis | G682 | Constitutive | Tolerance to | Increased tolerance to sucrose |
| | | thaliana | | 35S | hyperosmotic | |
| | | | | | stress | |
| G226 | 62 | Arabidopsis | G682 | Constitutive | Tolerance to | Increased tolerance to sucrose |
| | | thaliana | | 35S | hyperosmotic | |
| | | | | | stress | |
| G3392 | 72 | Oryza sativa | G682 | Constitutive | Tolerance to | Increased tolerance to |
| | | | | 35S | hyperosmotic | mannitol |
| | | | | | stress | |
| G682 | 60 | Arabidopsis | G682 | Vascular | Altered size | Increased biomass |
| | | thaliana | | SUC2 | | |
| G3393 | 66 | Oryza sativa | G682 | Constitutive | Altered root | Increased root hair |
| | | | | 35S | morphology | |
| G226 | 62 | Arabidopsis | G682 | Constitutive | Altered root | Increased root hair |
| | | thaliana | | 35S | morphology | |
| G682 | 60 | Arabidopsis | G682 | Constitutive | Altered root | Increased root hair |
| | | thaliana | | 35S | morphology | |
| G3392 | 72 | Oryza sativa | G682 | Constitutive | Altered root | Increased root hair |
| | | | | 35S | morphology | |
| G3431 | 68 | Zea mays | G682 | Constitutive | Altered root | Increased root hair |
| | | | | 35S | morphology | |
| G3444 | 70 | Zea mays | G682 | Constitutive | Altered root | Increased root hair |
| | | | | 35S | morphology | |
| G3448 | 80 | Glycine max | G682 | Constitutive | Altered root | Increased root hair |
| | | | | 35S | morphology | |
| G3449 | 78 | Glycine max | G682 | Constitutive | Altered root | Increased root hair |
| | | | | 35S | morphology | |
| G3450 | 70 | Glycine max | G682 | Constitutive | Altered root | Increased root hair |
| | | | | 35S | morphology | |
| G3392 | 72 | Oryza sativa | G682 | Constitutive | Altered seed | Pale seed color |
| | | | | 35S | morphology | |
| G3393 | 66 | Oryza sativa | G682 | Constitutive | Altered seed | Pale seed color |
| | | | | 35S | morphology | |
| G3431 | 68 | Zea mays | G682 | Constitutive | Altered seed | Pale seed color |
| | | | | 35S | morphology | |
| G3444 | 70 | Zea mays | G682 | Constitutive | Altered seed | Pale seed color |
| | | | | 35S | morphology | |
| G682 | 60 | Arabidopsis | G682 | RNAi (GS) | Tolerance to | Increased tolerance to NaCl |
| | | thaliana | | | sodium | |
| | | | | | chloride | |
| G1816 | 76 | Arabidopsis | G682 | KO | Tolerance to | Increased tolerance to NaCl |
| | | thaliana | | | sodium | |
| | | | | | chloride | |
| G682 | 60 | Arabidopsis | G682 | Epidermal | Tolerance to | Increased tolerance to NaCl |
| | | thaliana | | CUT1 | sodium | |
| | | | | | chloride | |
| G3392 | 72 | Oryza sativa | G682 | Constitutive | Tolerance to | Increased tolerance to NaCl |
| | | | | 35S | sodium | |
| | | | | | chloride | |
| G1816 | 76 | Arabidopsis | G682 | Constitutive | Altered sugar | Increased tolerance to sugar |
| | | thaliana | | 35S | sensing | |
| G2718 | 64 | Arabidopsis | G682 | Constitutive | Altered sugar | Increased tolerance to sugar |
| | | thaliana | | 35S | sensing | |
| G3392 | 72 | Oryza sativa | G682 | Constitutive | Altered sugar | Increased tolerance to sugar |
| | | | | 35S | sensing | |
| G3431 | 68 | Zea mays | G682 | Constitutive | Altered sugar | Increased tolerance to sugar |
| | | | | 35S | sensing | |
| G682 | 60 | Arabidopsis | G682 | Epidermal | Altered | Decreased trichome density |
| | | thaliana | | LTP1 | trichome | |
| | | | | | morphology | |
| G2718 | 64 | Arabidopsis | G682 | Constitutive | Altered | Decreased trichome density |
| | | thaliana | | 35S | trichome | |
| | | | | | morphology | |
| G3392 | 72 | Oryza sativa | G682 | Constitutive | Altered | Decreased trichome density |
| | | | | 35S | trichome | |
| | | | | | morphology | |
| G3393 | 66 | Oryza sativa | G682 | Constitutive | Altered | Decreased trichome density |
| | | | | 35S | trichome | |
| | | | | | morphology | |
| G3431 | 68 | Zea mays | G682 | Constitutive | Altered | Decreased trichome density |
| | | | | 35S | trichome | |
| | | | | | morphology | |
| G3444 | 70 | Zea mays | G682 | Constitutive | Altered | Decreased trichome density |
| | | | | 35S | trichome | |
| | | | | | morphology | |
| G3445 | 84 | Glycine max | G682 | Constitutive | Altered | Decreased trichome density |
| | | | | 35S | trichome | |
| | | | | | morphology | |
| G3446 | 82 | Glycine max | G682 | Constitutive | Altered | Decreased trichome density |
| | | | | 35S | trichome | |
| | | | | | morphology | |
| G3447 | 86 | Glycine max | G682 | Constitutive | Altered | Decreased trichome density |
| | | | | 35S | trichome | |
| | | | | | morphology | |
| G3448 | 80 | Glycine max | G682 | Constitutive | Altered | Decreased trichome density |
| | | | | 35S | trichome | |
| | | | | | morphology | |
| G3449 | 78 | Glycine max | G682 | Constitutive | Altered | Decreased trichome density |
| | | | | 35S | trichome | |
| | | | | | morphology | |
| G3450 | 70 | Glycine max | G682 | Constitutive | Altered | Decreased trichome density |
| | | | | 35S | trichome | |
| | | | | | morphology | |
| G9 | 106 | Arabidopsis | G867 | Constitutive | Cold tolerance | Increased tolerance to cold |
| | | thaliana | | 35S | | |
| G867 | 88 | Arabidopsis | G867 | Constitutive | Cold tolerance | Increased tolerance to cold |
| | | thaliana | | 35S | | |
| G867 | 88 | Arabidopsis | G867 | Deletion | Cold tolerance | Increased tolerance to cold |
| | | thaliana | | variant | | |
| G867 | 88 | Arabidopsis | G867 | GAL4 C- | Cold tolerance | Increased tolerance to cold |
| | | thaliana | | term (Super | | |
| | | | | Active) | | |
| G993 | 90 | Arabidopsis | G867 | Constitutive | Cold tolerance | Increased tolerance to cold |
| | | thaliana | | 35S | | |
| G1930 | 92 | Arabidopsis | G867 | Constitutive | Cold tolerance | Increased tolerance to cold |
| | | thaliana | | 35S | | |
| G3389 | 104 | Oryza sativa | G867 | Constitutive | Cold tolerance | Increased tolerance to cold |
| | | | | 35S | | |
| G3452 | 98 | Glycine max | G867 | Constitutive | Cold tolerance | Increased tolerance to cold |
| | | | | 35S | | |
| G867 | 88 | Arabidopsis | G867 | Root ARSK1 | Drought | Increased tolerance to drought |
| | | thaliana | | | tolerance | in soil assays |
| G867 | 88 | Arabidopsis | G867 | Vascular | Drought | Increased tolerance to |
| | | thaliana | | SUC2 | tolerance | dehydration |
| G867 | 88 | Arabidopsis | G867 | Deletion | Drought | Increased tolerance to |
| | | thaliana | | variant | tolerance | dehydration |
| G867 | 88 | Arabidopsis | G867 | GAL4 N- | Drought | Increased tolerance to drought |
| | | thaliana | | term (Super | tolerance | in soil assays |
| | | | | Active) | | |
| G867 | 88 | Arabidopsis | G867 | RNAi (clade) | Drought | Increased tolerance to drought |
| | | thaliana | | | tolerance | in soil assays |
| G867 | 88 | Arabidopsis | G867 | Stress | Drought | Increased tolerance to drought |
| | | thaliana | | Inducible | tolerance | in soil assays |
| | | | | RD29A | | |
| G867 | 88 | Arabidopsis | G867 | Vascular | Drought | Increased tolerance to drought |
| | | thaliana | | SUC2 | tolerance | in soil assays |
| G3389 | 104 | Oryza sativa | G867 | Constitutive | Drought | Increased tolerance to drought |
| | | | | 35S | tolerance | in soil assays |
| G3390 | 112 | Oryza sativa | G867 | Constitutive | Drought | Increased tolerance to |
| | | | | 35S | tolerance | dehydration |
| G3432 | 102 | Zea mays | G867 | Constitutive | Drought | Increased tolerance to drought |
| | | | | 35S | tolerance | in soil assays |
| G3451 | 108 | Glycine max | G867 | Constitutive | Drought | Increased tolerance to drought |
| | | | | 35S | tolerance | in soil assays |
| G867 | 88 | Arabidopsis | G867 | RNAi (clade) | Altered | Late flowering |
| | | thaliana | | | flowering time | |
| G3389 | 104 | Oryza sativa | G867 | Constitutive | Altered | Early flowering |
| | | | | 35S | flowering time | |
| G3389 | 104 | Oryza sativa | G867 | Constitutive | Heat tolerance | Increased tolerance to heat |
| | | | | 35S | | |
| G9 | 106 | Arabidopsis | G867 | Constitutive | Altered | Decreased ABA sensitivity |
| | | thaliana | | 35S | hormone | |
| | | | | | sensitivity | |
| G867 | 88 | Arabidopsis | G867 | Constitutive | Altered | Decreased ABA sensitivity |
| | | thaliana | | 35S | hormone | |
| | | | | | sensitivity | |
| G867 | 88 | Arabidopsis | G867 | Root ARSK1 | Altered | Decreased ABA sensitivity |
| | | thaliana | | | hormone | |
| | | | | | sensitivity | |
| G3390 | 112 | Oryza sativa | G867 | Constitutive | Altered | Decreased ABA sensitivity |
| | | | | 35S | hormone | |
| | | | | | sensitivity | |
| G3453 | 100 | Glycine max | G867 | Constitutive | Altered | Decreased ABA sensitivity |
| | | | | 35S | hormone | |
| | | | | | sensitivity | |
| G9 | 106 | Arabidopsis | G867 | Constitutive | Tolerance to | Increased tolerance to sucrose |
| | | thaliana | | 35S | hyperosmotic | |
| | | | | | stress | |
| G993 | 90 | Arabidopsis | G867 | Constitutive | Tolerance to | Increased tolerance to sucrose |
| | | thaliana | | 35S | hyperosmotic | |
| | | | | | stress | |
| G867 | 88 | Arabidopsis | G867 | Vascular | Tolerance to | Increased tolerance to sucrose |
| | | thaliana | | SUC2 | hyperosmotic | |
| | | | | | stress | |
| G3451 | 108 | Glycine max | G867 | Constitutive | Tolerance to | Increased tolerance to sucrose |
| | | | | 35S | hyperosmotic | |
| | | | | | stress | |
| G3452 | 98 | Glycine max | G867 | Constitutive | Tolerance to | Increased tolerance to sucrose |
| | | | | 35S | hyperosmotic | |
| | | | | | stress | |
| G9 | 106 | Arabidopsis | G867 | Constitutive | Altered root | Increased root hair |
| | | thaliana | | 35S | morphology | |
| G867 | 88 | Arabidopsis | G867 | Constitutive | Altered root | Increased root hair |
| | | thaliana | | 35S | morphology | |
| G993 | 90 | Arabidopsis | G867 | Constitutive | Altered root | Increased root hair |
| | | thaliana | | 35S | morphology | |
| G3451 | 108 | Glycine max | G867 | Constitutive | Altered root | Increased root hair |
| | | | | 35S | morphology | |
| G3452 | 98 | Glycine max | G867 | Constitutive | Altered root | Increased root hair |
| | | | | 35S | morphology | |
| G3455 | 96 | Glycine max | G867 | Constitutive | Altered root | Increased root hair |
| | | | | 35S | morphology | |
| G867 | 88 | Arabidopsis | G867 | RNAi (clade) | Altered size | Increased biomass |
| | | thaliana | | | | |
| G867 | 88 | Arabidopsis | G867 | GAL4 N- | Tolerance to | Increased tolerance to NaCl |
| | | thaliana | | term (Super | sodium | |
| | | | | Active) | chloride | |
| G867 | 88 | Arabidopsis | G867 | Leaf RBCS3 | Tolerance to | Increased tolerance to NaCl |
| | | thaliana | | | sodium | |
| | | | | | chloride | |
| G867 | 88 | Arabidopsis | G867 | Stress | Tolerance to | Increased tolerance to NaCl |
| | | thaliana | | Inducible | sodium | |
| | | | | RD29A | chloride | |
| G867 | 88 | Arabidopsis | G867 | Vascular | Tolerance to | Increased tolerance to NaCl |
| | | thaliana | | SUC2 | sodium | |
| | | | | | chloride | |
| G3389 | 104 | Oryza sativa | G867 | Constitutive | Tolerance to | Increased tolerance to NaCl |
| | | | | 35S | sodium | |
| | | | | | chloride | |
| G3391 | 94 | Oryza sativa | G867 | Constitutive | Tolerance to | Increased tolerance to NaCl |
| | | | | 35S | sodium | |
| | | | | | chloride | |
| G3452 | 98 | Glycine max | G867 | Constitutive | Tolerance to | Increased tolerance to NaCl |
| | | | | 35S | sodium | |
| | | | | | chloride | |
| G3456 | 132 | Glycine max | G867 | Constitutive | Tolerance to | Increased tolerance to NaCl |
| | | | | 35S | sodium | |
| | | | | | chloride | |
| G867 | 88 | Arabidopsis | G867 | GAL4 C- | Altered sugar | Increased tolerance to sugar |
| | | thaliana | | term (Super | sensing | |
| | | | | Active) | | |
| G3455 | 96 | Glycine max | G867 | Constitutive | Altered sugar | Increased tolerance to sugar |
| | | | | 35S | sensing | |
| G922 | 328 | Arabidopsis | G922 | Constitutive | Drought | Increased tolerance to |
| | | thaliana | | 35S | tolerance | dehydration |
| G922 | 328 | Arabidopsis | G922 | Constitutive | Drought | Increased tolerance to drought |
| | | thaliana | | 35S | tolerance | in soil assays |
| G922 | 328 | Arabidopsis | G922 | Constitutive | Tolerance to | Increased tolerance to NaCl |
| | | thaliana | | 35S | sodium | |
| | | | | | chloride | |
| G922 | 328 | Arabidopsis | G922 | Constitutive | Altered sugar | Increased tolerance to sugar |
| | | thaliana | | 35S | sensing | |
| G922 | 328 | Arabidopsis | G922 | Constitutive | Cold tolerance | Increased tolerance to cold |
| | | thaliana | | 35S | | |
| G922 | 328 | Arabidopsis | G922 | Constitutive | Altered | Decreased ABA sensitivity |
| | | thaliana | | 35S | hormone | |
| | | | | | sensitivity | |
| G1073 | 114 | Arabidopsis | G1073 | Floral | Cold tolerance | Increased tolerance to cold |
| | | thaliana | | meristem | | |
| | | | | AP1 | | |
| G1073 | 114 | Arabidopsis | G1073 | Double Over- | Cold tolerance | Increased tolerance to cold |
| | | thaliana | | expression | | |
| | | | | (with G481) | | |
| G2153 | 138 | Arabidopsis | G1073 | Constitutive | Cold tolerance | Increased tolerance to cold |
| | | thaliana | | 35S | | |
| G2156 | 130 | Arabidopsis | G1073 | Constitutive | Cold tolerance | Increased tolerance to cold |
| | | thaliana | | 35S | | |
| G3400 | 124 | Oryza sativa | G1073 | Constitutive | Cold tolerance | Increased tolerance to cold |
| | | | | 35S | | |
| G3456 | 132 | Glycine max | G1073 | Constitutive | Cold tolerance | Increased tolerance to cold |
| | | | | 35S | | |
| G3459 | 122 | Glycine max | G1073 | Constitutive | Cold tolerance | Increased tolerance to cold |
| | | | | 35S | | |
| G1073 | 114 | Arabidopsis | G1073 | Double Over- | Altered | Increased tolerance to low |
| | | thaliana | | expression | nutrient uptake | nitrogen conditions |
| | | | | (with G481) | | |
| G1073 | 114 | Arabidopsis | G1073 | Constitutive | Drought | Increased tolerance to drought |
| | | thaliana | | 35S | tolerance | in soil assays |
| G1073 | 114 | Arabidopsis | G1073 | Constitutive | Drought | Increased tolerance to |
| | | thaliana | | 35S | tolerance | dehydration |
| G1073 | 114 | Arabidopsis | G1073 | Shoot apical | Drought | Increased tolerance to |
| | | thaliana | | meristem | tolerance | dehydration |
| | | | | STM | | |
| G1073 | 114 | Arabidopsis | G1073 | Shoot apical | Drought | Increased tolerance to drought |
| | | thaliana | | meristem | tolerance | in soil assays |
| | | | | STM | | |
| G1073 | 114 | Arabidopsis | G1073 | GAL4 C- | Drought | Increased tolerance to |
| | | thaliana | | term (Super | tolerance | dehydration |
| | | | | Active) | | |
| G1073 | 114 | Arabidopsis | G1073 | GAL4 C- | Drought | Increased tolerance to drought |
| | | thaliana | | term (Super | tolerance | in soil assays |
| | | | | Active) | | |
| G1073 | 114 | Arabidopsis | G1073 | RNAi (GS) | Drought | Increased tolerance to |
| | | thaliana | | | tolerance | dehydration |
| G1073 | 114 | Arabidopsis | G1073 | RNAi (clade) | Drought | Increased tolerance to |
| | | thaliana | | | tolerance | dehydration |
| G1067 | 120 | Arabidopsis | G1073 | Constitutive | Drought | Increased tolerance to drought |
| | | thaliana | | 35S | tolerance | in soil assays |
| G1067 | 120 | Arabidopsis | G1073 | stress | Drought | Increased tolerance to |
| | | thaliana | | Inducible | tolerance | dehydration |
| | | | | RD29A | | |
| G1067 | 120 | Arabidopsis | G1073 | stress | Drought | Increased tolerance to drought |
| | | thaliana | | Inducible | tolerance | in soil assays |
| | | | | RD29A | | |
| G1067 | 120 | Arabidopsis | G1073 | Root ARSK1 | Drought | Increased tolerance to |
| | | thaliana | | | tolerance | dehydration |
| G2153 | 138 | Arabidopsis | G1073 | Constitutive | Drought | Increased tolerance to drought |
| | | thaliana | | 35S | tolerance | in soil assays |
| G2156 | 130 | Arabidopsis | G1073 | Constitutive | Drought | Increased tolerance to drought |
| | | thaliana | | 35S | tolerance | in soil assays |
| G2156 | 130 | Arabidopsis | G1073 | Root ARSK1 | Drought | Increased tolerance to |
| | | thaliana | | | tolerance | dehydration |
| G2157 | 144 | Arabidopsis | G1073 | Constitutive | Drought | Increased tolerance to |
| | | thaliana | | 35S | tolerance | dehydration |
| G3399 | 118 | Oryza sativa | G1073 | Constitutive | Drought | Increased tolerance to |
| | | | | 35S | tolerance | dehydration |
| G3399 | 118 | Oryza sativa | G1073 | Constitutive | Drought | Increased tolerance to drought |
| | | | | 35S | tolerance | in soil assays |
| G3400 | 124 | Oryza sativa | G1073 | Constitutive | Drought | Increased tolerance to drought |
| | | | | 35S | tolerance | in soil assays |
| G3401 | 136 | Oryza sativa | G1073 | Constitutive | Drought | Increased tolerance to drought |
| | | | | 35S | tolerance | in soil assays |
| G3408 | 146 | Oryza sativa | G1073 | Constitutive | Drought | Increased tolerance to drought |
| | | | | 35S | tolerance | in soil assays |
| G3456 | 132 | Glycine max | G1073 | Constitutive | Drought | Increased tolerance to drought |
| | | | | 35S | tolerance | in soil assays |
| G3460 | 126 | Glycine max | G1073 | Constitutive | Drought | Increased tolerance to drought |
| | | | | 35S | tolerance | in soil assays |
| G3556 | 142 | Oryza sativa | G1073 | Constitutive | Drought | Increased tolerance to |
| | | | | 35S | tolerance | dehydration |
| G1073 | 114 | Arabidopsis | G1073 | Constitutive | Altered flower | Large flower |
| | | thaliana | | 35S | morphology | |
| G2153 | 138 | Arabidopsis | G1073 | Constitutive | Altered flower | Large flower |
| | | thaliana | | 35S | morphology | |
| G2156 | 130 | Arabidopsis | G1073 | Constitutive | Altered flower | Large flower |
| | | thaliana | | 35S | morphology | |
| G3399 | 118 | Oryza sativa | G1073 | Constitutive | Altered flower | Large flower |
| | | | | 35S | morphology | |
| G3400 | 124 | Oryza sativa | G1073 | Constitutive | Altered flower | Large flower |
| | | | | 35S | morphology | |
| G2153 | 138 | Arabidopsis | G1073 | Constitutive | Altered | Late flowering |
| | | thaliana | | 35S | flowering time | |
| G2156 | 130 | Arabidopsis | G1073 | Constitutive | Altered | Late flowering |
| | | thaliana | | 35S | flowering time | |
| G2156 | 130 | Arabidopsis | G1073 | Root ARSK1 | Altered | Late flowering |
| | | thaliana | | | flowering time | |
| G3399 | 118 | Oryza sativa | G1073 | Constitutive | Altered | Late flowering |
| | | | | 35S | flowering time | |
| G3400 | 124 | Oryza sativa | G1073 | Constitutive | Altered | Late flowering |
| | | | | 35S | flowering time | |
| G3406 | 116 | Oryza sativa | G1073 | Constitutive | Heat tolerance | Increased tolerance to heat |
| | | | | 35S | | |
| G3459 | 122 | Glycine max | G1073 | Constitutive | Heat tolerance | Increased tolerance to heat |
| | | | | 35S | | |
| G3460 | 126 | Glycine max | G1073 | Constitutive | Heat tolerance | Increased tolerance to heat |
| | | | | 35S | | |
| G2153 | 138 | Arabidopsis | G1073 | Constitutive | Altered | Decreased ABA sensitivity |
| | | thaliana | | 35S | hormone | |
| | | | | | sensitivity | |
| G3406 | 116 | Oryza sativa | G1073 | Constitutive | Altered | Decreased ABA sensitivity |
| | | | | 35S | hormone | |
| | | | | | sensitivity | |
| G2156 | 130 | Arabidopsis | G1073 | Leaf RBCS3 | Altered leaf | Large leaf size |
| | | thaliana | | | morphology | |
| G1067 | 120 | Arabidopsis | G1073 | Leaf RBCS3 | Altered leaf | Large leaf size |
| | | thaliana | | | morphology | |
| G1067 | 120 | Arabidopsis | G1073 | Stress | Altered leaf | Large leaf size |
| | | thaliana | | Inducible | morphology | |
| | | | | RD29A | | |
| G2156 | 130 | Arabidopsis | G1073 | Constitutive | Altered leaf | Large leaf size |
| | | thaliana | | 35S | morphology | |
| G2157 | 144 | Arabidopsis | G1073 | Constitutive | Altered leaf | Altered leaf shape |
| | | thaliana | | 35S | morphology | |
| G2157 | 144 | Arabidopsis | G1073 | Constitutive | Altered leaf | Large leaf size |
| | | thaliana | | 35S | morphology | |
| G3399 | 118 | Oryza sativa | G1073 | Constitutive | Altered leaf | Large leaf size |
| | | | | 35S | morphology | |
| G3400 | 124 | Oryza sativa | G1073 | Constitutive | Altered leaf | Altered leaf shape (short |
| | | | | 35S | morphology | rounded curled leaves at early |
| | | | | | | stages, broad leaves at later |
| | | | | | | stages) |
| G3400 | 124 | Oryza sativa | G1073 | Constitutive | Altered leaf | Large leaf size |
| | | | | 35S | morphology | |
| G3456 | 132 | Glycine max | G1073 | Constitutive | Altered leaf | Dark green leaf color |
| | | | | 35S | morphology | |
| G3456 | 132 | Glycine max | G1073 | Constitutive | Altered leaf | Large leaf size |
| | | | | 35S | morphology | |
| G3460 | 126 | Glycine max | G1073 | Constitutive | Altered leaf | Dark green leaf color |
| | | | | 35S | morphology | |
| G3407 | 134 | Oryza sativa | G1073 | Constitutive | Altered | Increased seedling size |
| | | | | 35S | morphology | |
| G1073 | 114 | Arabidopsis | G1073 | Constitutive | Tolerance to | Increased tolerance to sucrose |
| | | thaliana | | 35S | hyperosmotic | |
| | | | | | stress | |
| G1067 | 120 | Arabidopsis | G1073 | Stress | Tolerance to | Increased tolerance to |
| | | thaliana | | Inducible | hyperosmotic | hyperosmotic stress |
| | | | | RD29A | stress | |
| G1073 | 114 | Arabidopsis | G1073 | Epidermal | Tolerance to | Increased tolerance to sucrose |
| | | thaliana | | CUT1 | hyperosmotic | and mannitol |
| | | | | | stress | |
| G1067 | 120 | Arabidopsis | G1073 | Stress | Altered root | Increased root hair |
| | | thaliana | | Inducible | morphology | |
| | | | | RD29A | | |
| G1073 | 114 | Arabidopsis | G1073 | Constitutive | Altered root | Altered root branching |
| | | thaliana | | 35S | morphology | |
| G1073 | 114 | Arabidopsis | G1073 | Constitutive | Altered root | Increased root mass |
| | | thaliana | | 35S | morphology | |
| G1073 | 114 | Arabidopsis | G1073 | Constitutive | Altered root | Increased root hair |
| | | thaliana | | 35S | morphology | |
| G3399 | 118 | Oryza sativa | G1073 | Constitutive | Altered root | Increased root hair |
| | | | | 35S | morphology | |
| G3399 | 118 | Oryza sativa | G1073 | Constitutive | Altered root | Increased root mass |
| | | | | 35S | morphology | |
| G3456 | 132 | Glycine max | G1073 | Constitutive | Altered | Late senescence |
| | | | | 35S | senescence | |
| G1073 | 114 | Arabidopsis | G1073 | Double Over- | Altered size | Increased biomass |
| | | thaliana | | expression | | |
| | | | | (with G481) | | |
| G2156 | 130 | Arabidopsis | G1073 | Leaf RBCS3 | Altered size | Increased biomass |
| | | thaliana | | | | |
| G3399 | 118 | Oryza sativa | G1073 | Constitutive | Altered size | Increased biomass |
| | | | | 35S | | |
| G3400 | 124 | Oryza sativa | G1073 | Constitutive | Altered size | Increased biomass |
| | | | | 35S | | |
| G3460 | 126 | Glycine max | G1073 | Constitutive | Altered size | Increased biomass |
| | | | | 35S | | |
| G1073 | 114 | Arabidopsis | G1073 | Deletion | Altered size | Increased biomass |
| | | thaliana | | variant | | |
| G1073 | 114 | Arabidopsis | G1073 | Vascular | Altered size | Increased biomass |
| | | thaliana | | SUC2 | | |
| G2153 | 138 | Arabidopsis | G1073 | Constitutive | Altered size | Increased biomass |
| | | thaliana | | 35S | | |
| G2156 | 130 | Arabidopsis | G1073 | Constitutive | Altered size | Increased biomass |
| | | thaliana | | 35S | | |
| G3456 | 132 | Glycine max | G1073 | Constitutive | Altered size | Increased biomass |
| | | | | 35S | | |
| G1073 | 114 | Arabidopsis | G1073 | Constitutive | Tolerance to | Increased tolerance to NaCl |
| | | thaliana | | 35S | sodium | |
| | | | | | chloride | |
| G2156 | 130 | Arabidopsis | G1073 | Constitutive | Tolerance to | Increased tolerance to NaCl |
| | | thaliana | | 35S | sodium | |
| | | | | | chloride | |
| G1067 | 120 | Arabidopsis | G1073 | Root ARSK1 | Tolerance to | Increased tolerance to NaCl |
| | | thaliana | | | sodium | |
| | | | | | chloride | |
| G1067 | 120 | Arabidopsis | G1073 | Leaf RBCS3 | Tolerance to | Increased tolerance to NaCl |
| | | thaliana | | | sodium | |
| | | | | | chloride | |
| G1067 | 120 | Arabidopsis | G1073 | Stress | Tolerance to | Increased tolerance to NaCl |
| | | thaliana | | Inducible | sodium | |
| | | | | RD29A | chloride | |
| G1073 | 114 | Arabidopsis | G1073 | Root ARSK1 | Tolerance to | Increased tolerance to NaCl |
| | | thaliana | | | sodium | |
| | | | | | chloride | |
| G3401 | 136 | Oryza sativa | G1073 | Constitutive | Tolerance to | Increased tolerance to NaCl |
| | | | | 35S | sodium | |
| | | | | | chloride | |
| G3459 | 122 | Glycine max | G1073 | Constitutive | Tolerance to | Increased tolerance to NaCl |
| | | | | 35S | sodium | |
| | | | | | chloride | |
| G3556 | 142 | Oryza sativa | G1073 | Constitutive | Tolerance to | Increased tolerance to NaCl |
| | | | | 35S | sodium | |
| | | | | | chloride | |
| G1073 | 114 | Arabidopsis | G1073 | Constitutive | Altered sugar | Increased tolerance to sugar |
| | | thaliana | | 35S | sensing | |
| G2156 | 130 | Arabidopsis | G1073 | Constitutive | Altered sugar | Increased tolerance to sugar |
| | | thaliana | | 35S | sensing | |
| G3401 | 136 | Oryza sativa | G1073 | Constitutive | Altered sugar | Increased tolerance to sugar |
| | | | | 35S | sensing | |
| G1274 | 186 | Arabidopsis | G1274 | GAL4 C- | Altered | Inflorescence: decreased |
| | | thaliana | | term (Super | architecture | apical dominance |
| | | | | Active) | | |
| G1274 | 186 | Arabidopsis | G1274 | Point | Altered | Inflorescence: decreased |
| | | thaliana | | mutation | architecture | apical dominance |
| G1274 | 186 | Arabidopsis | G1274 | Point | Altered | Altered C/N sensing: |
| | | thaliana | | mutation | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3720 | 204 | Zea mays | G1274 | Constitutive | Altered | Increased tolerance to low |
| | | | | 35S | nutrient uptake | nitrogen conditions |
| G3722 | 200 | Zea mays | G1274 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3727 | 196 | Zea mays | G1274 | Constitutive | Altered | Increased tolerance to low |
| | | | | 35S | nutrient uptake | nitrogen conditions |
| G3729 | 216 | Oryza sativa | G1274 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3721 | 198 | Oryza sativa | G1274 | Constitutive | Tolerance to | Increased tolerance to |
| | | | | 35S | hyperosmotic | mannitol |
| | | | | | stress | |
| G1274 | 186 | Arabidopsis | G1274 | Constitutive | Drought | Increased tolerance to |
| | | thaliana | | 35S | tolerance | dehydration |
| G1274 | 186 | Arabidopsis | G1274 | Point | Drought | Increased tolerance to drought |
| | | thaliana | | mutation | tolerance | in soil assays |
| G1275 | 208 | Arabidopsis | G1274 | Constitutive | Drought | Increased tolerance to drought |
| | | thaliana | | 35S | tolerance | in soil assays |
| G1275 | 208 | Arabidopsis | G1274 | Stress | Drought | Increased tolerance to drought |
| | | thaliana | | Inducible | tolerance | in soil assays |
| | | | | RD29A | | |
| G3803 | 194 | Glycine max | G1274 | Constitutive | Drought | Increased tolerance to |
| | | | | 35S | tolerance | dehydration |
| G3719 | 212 | Zea mays | G1274 | Constitutive | Altered | Inflorescence: decreased |
| | | | | 35S | architecture | apical dominance |
| G3720 | 204 | Zea mays | G1274 | Constitutive | Altered | Inflorescence: decreased |
| | | | | 35S | architecture | apical dominance |
| G3721 | 198 | Oryza sativa | G1274 | Constitutive | Altered | Inflorescence: decreased |
| | | | | 35S | architecture | apical dominance |
| G3722 | 200 | Zea mays | G1274 | Constitutive | Altered | Inflorescence: decreased |
| | | | | 35S | architecture | apical dominance |
| G3726 | 202 | Oryza sativa | G1274 | Constitutive | Altered | Inflorescence: decreased |
| | | | | 35S | architecture | apical dominance |
| G1274 | 186 | Arabidopsis | G1274 | Constitutive | Cold tolerance | Increased tolerance to cold |
| | | thaliana | | 35S | | |
| G1274 | 186 | Arabidopsis | G1274 | Point | Cold tolerance | Increased tolerance to cold |
| | | thaliana | | mutation | | |
| G1275 | 208 | Arabidopsis | G1274 | Constitutive | Cold tolerance | Increased tolerance to cold |
| | | thaliana | | 35S | | |
| G1758 | 394 | Arabidopsis | G1274 | Constitutive | Cold tolerance | Increased tolerance to cold |
| | | thaliana | | 35S | | |
| G3721 | 198 | Oryza sativa | G1274 | Constitutive | Cold tolerance | Increased tolerance to cold |
| | | | | 35S | | |
| G3726 | 202 | Oryza sativa | G1274 | Constitutive | Cold tolerance | Increased tolerance to cold |
| | | | | 35S | | |
| G3729 | 216 | Oryza sativa | G1274 | Constitutive | Cold tolerance | Increased tolerance to cold |
| | | | | 35S | | |
| G3804 | 192 | Zea mays | G1274 | Constitutive | Cold tolerance | Increased tolerance to cold |
| | | | | 35S | | |
| G194 | 218 | Arabidopsis | G1274 | Constitutive | Drought | Increased tolerance to |
| | | thaliana | | 35S | tolerance | dehydration |
| G2517 | 220 | Arabidopsis | G1274 | Constitutive | Drought | Increased tolerance to |
| | | thaliana | | 35S | tolerance | dehydration |
| G3804 | 192 | Zea mays | G1274 | Constitutive | Drought | Increased tolerance to drought |
| | | | | 35S | tolerance | in soil assays |
| G2517 | 220 | Arabidopsis | G1274 | Constitutive | Altered | Early flowering |
| | | thaliana | | 35S | flowering time | |
| G1275 | 208 | Arabidopsis | G1274 | Constitutive | Heat tolerance | Increased tolerance to heat |
| | | thaliana | | 35S | | |
| G1274 | 186 | Arabidopsis | G1274 | Constitutive | Altered | Decreased ABA sensitivity |
| | | thaliana | | 35S | hormone | |
| | | | | | sensitivity | |
| G1275 | 208 | Arabidopsis | G1274 | Stress | Altered | Decreased ABA sensitivity |
| | | thaliana | | Inducible | hormone | |
| | | | | RD29A | sensitivity | |
| G3721 | 198 | Oryza sativa | G1274 | Constitutive | Altered | Decreased ABA sensitivity |
| | | | | 35S | hormone | |
| | | | | | sensitivity | |
| G3721 | 198 | Oryza sativa | G1274 | Constitutive | Tolerance to | Increased tolerance to NaCl |
| | | | | 35S | sodium | |
| | | | | | chloride | |
| G1274 | 186 | Arabidopsis | G1274 | Point | Altered leaf | Large leaf size |
| | | thaliana | | mutation | morphology | |
| G1274 | 186 | Arabidopsis | G1274 | GAL4 C- | Altered leaf | Large leaf size |
| | | thaliana | | term (Super | morphology | |
| | | | | Active) | | |
| G3724 | 188 | Glycine max | G1274 | Constitutive | Altered leaf | Large leaf size |
| | | | | 35S | morphology | |
| G3725 | 214 | Oryza sativa | G1274 | Constitutive | Altered root | Increased root mass |
| | | | | 35S | morphology | |
| G1274 | 186 | Arabidopsis | G1274 | Constitutive | Silique | Increased seed number |
| | | thaliana | | 35S | | |
| G1274 | 186 | Arabidopsis | G1274 | Constitutive | Silique | Trilocular silique |
| | | thaliana | | 35S | | |
| G3724 | 188 | Glycine max | G1274 | Constitutive | Altered size | Increased biomass |
| | | | | 35S | | |
| G1274 | 186 | Arabidopsis | G1274 | Constitutive | Altered sugar | Increased tolerance to sugar |
| | | thaliana | | 35S | sensing | |
| G1274 | 186 | Arabidopsis | G1274 | Point | Altered sugar | Increased tolerance to sugar |
| | | thaliana | | mutation | sensing | |
| G30 | 226 | Arabidopsis | G1792 | Dex induced | Resistance to | Increased resistance to |
| | | thaliana | | | Botrytis | Botrytis |
| G30 | 226 | Arabidopsis | G1792 | Leaf RBCS3 | Resistance to | Increased resistance to |
| | | thaliana | | | Botrytis | Botrytis |
| G1266 | 254 | Arabidopsis | G1792 | Constitutive | Resistance to | Increased resistance to |
| | | thaliana | | 35S | Botrytis | Botrytis |
| G1791 | 230 | Arabidopsis | G1792 | Dex induced | Resistance to | Increased resistance to |
| | | thaliana | | | Botrytis | Botrytis |
| G1792 | 222 | Arabidopsis | G1792 | Dex induced | Resistance to | Increased resistance to |
| | | thaliana | | | Botrytis | Botrytis |
| G1792 | 222 | Arabidopsis | G1792 | Leaf RBCS3 | Resistance to | Increased resistance to |
| | | thaliana | | | Botrytis | Botrytis |
| G1795 | 224 | Arabidopsis | G1792 | Epidermal | Resistance to | Increased resistance to |
| | | thaliana | | LTP1 | Botrytis | Botrytis |
| G1795 | 224 | Arabidopsis | G1792 | Leaf RBCS3 | Resistance to | Increased resistance to |
| | | thaliana | | | Botrytis | Botrytis |
| G1791 | 230 | Arabidopsis | G1792 | Epidermal | Resistance to | Increased resistance to |
| | | thaliana | | LTP1 | Botrytis | Botrytis |
| G1792 | 222 | Arabidopsis | G1792 | Constitutive | Cold tolerance | Increased tolerance to cold |
| | | thaliana | | 35S | | |
| G3380 | 250 | Oryza sativa | G1792 | Constitutive | Cold tolerance | Increased tolerance to cold |
| | | | | 35S | | |
| G3381 | 234 | Oryza sativa | G1792 | Constitutive | Cold tolerance | Increased tolerance to cold |
| | | | | 35S | | |
| G3383 | 228 | Oryza sativa | G1792 | Constitutive | Cold tolerance | Increased tolerance to cold |
| | | | | 35S | | |
| G3516 | 240 | Zea mays | G1792 | Constitutive | Cold tolerance | Increased tolerance to cold |
| | | | | 35S | | |
| G3517 | 244 | Zea mays | G1792 | Constitutive | Cold tolerance | Increased tolerance to cold |
| | | | | 35S | | |
| G3518 | 246 | Glycine max | G1792 | Constitutive | Cold tolerance | Increased tolerance to cold |
| | | | | 35S | | |
| G3724 | 188 | Glycine max | G1792 | Constitutive | Cold tolerance | Increased tolerance to cold |
| | | | | 35S | | |
| G3737 | 236 | Oryza sativa | G1792 | Constitutive | Cold tolerance | Increased tolerance to cold |
| | | | | 35S | | |
| G3739 | 248 | Zea mays | G1792 | Constitutive | Cold tolerance | Increased tolerance to cold |
| | | | | 35S | | |
| G3794 | 252 | Zea mays | G1792 | Constitutive | Cold tolerance | Increased tolerance to cold |
| | | | | 35S | | |
| G1791 | 230 | Arabidopsis | G1792 | Epidermal | Drought | Increased tolerance to |
| | | thaliana | | CUT1 | tolerance | dehydration |
| G1795 | 224 | Arabidopsis | G1792 | Vascular | Drought | Increased tolerance to |
| | | thaliana | | SUC2 | tolerance | dehydration |
| G3380 | 250 | Oryza sativa | G1792 | Constitutive | Drought | Increased tolerance to drought |
| | | | | 35S | tolerance | in soil assays |
| G3383 | 228 | Oryza sativa | G1792 | Constitutive | Drought | Increased tolerance to |
| | | | | 35S | tolerance | dehydration |
| G3515 | 238 | Oryza sativa | G1792 | Constitutive | Drought | Increased tolerance to drought |
| | | | | 35S | tolerance | in soil assays |
| G3518 | 246 | Glycine max | G1792 | Constitutive | Drought | Increased tolerance to drought |
| | | | | 35S | tolerance | in soil assays |
| G3737 | 236 | Zea mays | G1792 | Constitutive | Drought | Increased tolerance to |
| | | | | 35S | tolerance | dehydration |
| G3737 | 236 | Zea mays | G1792 | Constitutive | Drought | Increased tolerance to drought |
| | | | | 35S | tolerance | in soil assays |
| G3739 | 248 | Zea mays | G1792 | Constitutive | Drought | Increased tolerance to |
| | | | | 35S | tolerance | dehydration |
| G3794 | 252 | Zea mays | G1792 | Constitutive | Drought | Increased tolerance to |
| | | | | 35S | tolerance | dehydration |
| G1791 | 230 | Arabidopsis | G1792 | Vascular | Altered | Late flowering |
| | | thaliana | | SUC2 | flowering time | |
| G3517 | 244 | Zea mays | G1792 | Constitutive | Heat tolerance | Increased tolerance to heat |
| | | | | 35S | | |
| G1266 | 254 | Arabidopsis | G1792 | Constitutive | Altered | Decreased ABA sensitivity |
| | | thaliana | | 35S | hormone | |
| | | | | | sensitivity | |
| G1791 | 230 | Arabidopsis | G1792 | Leaf RBCS3 | Altered | Decreased ABA sensitivity |
| | | thaliana | | | hormone | |
| | | | | | sensitivity | |
| G1795 | 224 | Arabidopsis | G1792 | Vascular | Altered | Decreased ABA sensitivity |
| | | thaliana | | SUC2 | hormone | |
| | | | | | sensitivity | |
| G3518 | 246 | Glycine max | G1792 | Constitutive | Altered | Decreased ABA sensitivity |
| | | | | 35S | hormone | |
| | | | | | sensitivity | |
| G3724 | 188 | Glycine max | G1792 | Constitutive | Altered | Decreased ABA sensitivity |
| | | | | 35S | hormone | |
| | | | | | sensitivity | |
| G3737 | 236 | Zea mays | G1792 | Constitutive | Altered | Decreased ABA sensitivity |
| | | | | 35S | hormone | |
| | | | | | sensitivity | |
| G3739 | 248 | Zea mays | G1792 | Constitutive | Altered | Decreased ABA sensitivity |
| | | | | 35S | hormone | |
| | | | | | sensitivity | |
| G3380 | 250 | Oryza sativa | G1792 | Constitutive | Altered | Decreased ABA sensitivity |
| | | | | 35S | hormone | |
| | | | | | sensitivity | |
| G30 | 226 | Arabidopsis | G1792 | Vascular | Altered leaf | Glossy leaves |
| | | thaliana | | SUC2 | morphology | |
| G1792 | 222 | Arabidopsis | G1792 | Point | Altered leaf | Gray leaf color |
| | | thaliana | | mutation | morphology | |
| G30 | 226 | Arabidopsis | G1792 | AS1 | Light response | Altered leaf orientation |
| | | thaliana | | | | (upward pointing cotyledons) |
| G1752 | 402 | Arabidopsis | G1792 | Constitutive | Altered | Altered C/N sensing: |
| | | thaliana | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G1792 | 222 | Arabidopsis | G1792 | Constitutive | Altered | Altered C/N sensing: |
| | | thaliana | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G30 | 226 | Arabidopsis | G1792 | Epidermal | Altered | Increased tolerance to low |
| | | thaliana | | LTP1 | nutrient uptake | nitrogen conditions |
| G1795 | 224 | Arabidopsis | G1792 | Vascular | Altered | Increased tolerance to low |
| | | thaliana | | SUC2 | nutrient uptake | nitrogen conditions |
| G3516 | 240 | Zea mays | G1792 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G3520 | 242 | Glycine max | G1792 | Constitutive | Altered | Altered C/N sensing: |
| | | | | 35S | nutrient uptake | increased tolerance to basal |
| | | | | | | media minus nitrogen plus 3% |
| | | | | | | sucrose and/or basal media |
| | | | | | | minus nitrogen plus 3% |
| | | | | | | sucrose and 1 mM glutamine |
| G1752 | 402 | Arabidopsis | G1792 | Constitutive | Tolerance to | Increased tolerance to |
| | | thaliana | | 35S | hyperosmotic | mannitol |
| | | | | | stress | |
| G1795 | 224 | Arabidopsis | G1792 | Vascular | Tolerance to | Increased tolerance to |
| | | thaliana | | SUC2 | hyperosmotic | mannitol |
| | | | | | stress | |
| G3380 | 250 | Oryza sativa | G1792 | Constitutive | Tolerance to | Increased tolerance to |
| | | | | 35S | hyperosmotic | mannitol |
| | | | | | stress | |
| G3739 | 248 | Zea mays | G1792 | Constitutive | Tolerance to | Increased tolerance to |
| | | | | 35S | hyperosmotic | mannitol |
| | | | | | stress | |
| G3381 | 234 | Oryza sativa | G1792 | Constitutive | Tolerance to | Increased tolerance to |
| | | | | 35S | hyperosmotic | mannitol |
| | | | | | stress | |
| G3383 | 228 | Oryza sativa | G1792 | Constitutive | Tolerance to | Increased tolerance to |
| | | | | 35S | hyperosmotic | mannitol |
| | | | | | stress | |
| G3519 | 232 | Glycine max | G1792 | Constitutive | Tolerance to | Increased tolerance to |
| | | | | 35S | hyperosmotic | mannitol |
| | | | | | stress | |
| G1792 | 222 | Arabidopsis | G1792 | Constitutive | Altered root | Increased root hair |
| | | thaliana | | 35S | morphology | |
| G1792 | 222 | Arabidopsis | G1792 | Constitutive | Altered root | Increased root mass |
| | | thaliana | | 35S | morphology | |
| G3515 | 238 | Oryza sativa | G1792 | Constitutive | Altered root | Increased root hair |
| | | | | 35S | morphology | |
| G3515 | 238 | Oryza sativa | G1792 | Constitutive | Altered root | Increased root mass |
| | | | | 35S | morphology | |
| G30 | 226 | Arabidopsis | G1792 | Dex induced | Resistance to | Increased resistance to |
| | | thaliana | | | Sclerotinia | Sclerotinia |
| G30 | 226 | Arabidopsis | G1792 | Leaf RBCS3 | Resistance to | Increased resistance to |
| | | thaliana | | | Sclerotinia | Sclerotinia |
| G1791 | 230 | Arabidopsis | G1792 | Dex induced | Resistance to | Increased resistance to |
| | | thaliana | | | Sclerotinia | Sclerotinia |
| G1795 | 224 | Arabidopsis | G1792 | Epidermal | Resistance to | Increased resistance to |
| | | thaliana | | LTP1 | Sclerotinia | Sclerotinia |
| G1795 | 224 | Arabidopsis | G1792 | Leaf RBCS3 | Resistance to | Increased resistance to |
| | | thaliana | | | Sclerotinia | Sclerotinia |
| G1795 | 224 | Arabidopsis | G1792 | Epidermal- | Resistance to | Increased resistance to |
| | | thaliana | | specific | Sclerotinia | Sclerotinia |
| | | | | CUT1 | | |
| G1266 | 254 | Arabidopsis | G1792 | Constitutive | Resistance to | Increased resistance to |
| | | thaliana | | 35S | Sclerotinia | Sclerotinia |
| G3381 | 234 | Oryza sativa | G1792 | Constitutive | Resistance to | Increased resistance to |
| | | | | 35S | Sclerotinia | Sclerotinia |
| G3518 | 246 | Glycine max | G1792 | Constitutive | Tolerance to | Increased tolerance to NaCl |
| | | | | 35S | sodium | |
| | | | | | chloride | |
| G3724 | 188 | Glycine max | G1792 | Constitutive | Tolerance to | Increased tolerance to NaCl |
| | | | | 35S | sodium | |
| | | | | | chloride | |
| G3737 | 236 | Oryza sativa | G1792 | Constitutive | Tolerance to | Increased tolerance to NaCl |
| | | | | 35S | sodium | |
| | | | | | chloride | |
| G3724 | 188 | Glycine max | G1792 | Constitutive | Tolerance to | Increased tolerance to sugar |
| | | | | 35S | hyperosmotic | |
| | | | | | stress | |
| G3739 | 248 | Zea mays | G1792 | Constitutive | Tolerance to | Increased tolerance to sugar |
| | | | | 35S | hyperosmotic | |
| | | | | | stress | |
| G2053 | 330 | Arabidopsis | G2053 | Constitutive | Altered | Early flowering |
| | | thaliana | | 35S | flowering time | |
| G2053 | 330 | Arabidopsis | G2053 | Constitutive | Drought | Increased tolerance to drought |
| | | thaliana | | 35S | tolerance | in soil assays |
| G516 | 334 | Arabidopsis | G2053 | Constitutive | Tolerance to | Increased tolerance to |
| | | thaliana | | 35S | hyperosmotic | mannitol |
| | | | | | stress | |
| G516 | 334 | Arabidopsis | G2053 | Constitutive | Cold tolerance | Increased tolerance to cold |
| | | thaliana | | 35S | | |
| G2999 | 256 | Arabidopsis | G2999 | Constitutive | Cold tolerance | Increased tolerance to cold |
| | | thaliana | | 35S | | |
| G2989 | 280 | Arabidopsis | G2999 | Constitutive | Cold tolerance | Increased tolerance to cold |
| | | thaliana | | 35S | | |
| G2990 | 284 | Arabidopsis | G2999 | Constitutive | Cold tolerance | Increased tolerance to cold |
| | | thaliana | | 35S | | |
| G2992 | 286 | Arabidopsis | G2999 | Constitutive | Cold tolerance | Increased tolerance to cold |
| | | thaliana | | 35S | | |
| G2997 | 264 | Arabidopsis | G2999 | Constitutive | Cold tolerance | Increased tolerance to cold |
| | | thaliana | | 35S | | |
| G3002 | 290 | Arabidopsis | G2999 | Constitutive | Cold tolerance | Increased tolerance to cold |
| | | thaliana | | 35S | | |
| G3685 | 274 | Oryza sativa | G2999 | Constitutive | Cold tolerance | Increased tolerance to cold |
| | | | | 35S | | |
| G3686 | 268 | Oryza sativa | G2999 | Constitutive | Cold tolerance | Increased tolerance to cold |
| | | | | 35S | | |
| G2989 | 280 | Arabidopsis | G2999 | Constitutive | Drought | Increased tolerance to drought |
| | | thaliana | | 35S | tolerance | in soil assays |
| G2989 | 280 | Arabidopsis | G2999 | Constitutive | Drought | Increased tolerance to |
| | | thaliana | | 35S | tolerance | dehydration |
| G2989 | 280 | Arabidopsis | G2999 | Constitutive | Drought | Increased tolerance to drought |
| | | thaliana | | 35S | tolerance | in soil assays |
| G2990 | 284 | Arabidopsis | G2999 | Constitutive | Drought | Increased tolerance to drought |
| | | thaliana | | 35S | tolerance | in soil assays |
| G3676 | 266 | Zea mays | G2999 | Constitutive | Drought | Increased tolerance to |
| | | | | 35S | tolerance | dehydration |
| G3686 | 268 | Oryza sativa | G2999 | Constitutive | Drought | Increased tolerance to drought |
| | | | | 35S | tolerance | in soil assays |
| G3002 | 290 | Arabidopsis | G2999 | Constitutive | Heat tolerance | Increased tolerance to heat |
| | | thaliana | | 35S | | |
| G3690 | 262 | Oryza sativa | G2999 | Constitutive | Heat tolerance | Increased tolerance to heat |
| | | | | 35S | | |
| G2999 | 256 | Arabidopsis | G2999 | GAL4 N- | Altered | Early flowering |
| | | thaliana | | term (Super | flowering time | |
| | | | | Active) | | |
| G3000 | 260 | Arabidopsis | G2999 | Constitutive | Altered | Early flowering |
| | | thaliana | | 35S | flowering time | |
| G3676 | 266 | Zea mays | G2999 | Constitutive | Altered | Early flowering |
| | | | | 35S | flowering time | |
| G3686 | 286 | Oryza sativa | G2999 | Constitutive | Altered | Early flowering |
| | | | | 35S | flowering time | |
| G2990 | 284 | Arabidopsis | G2999 | Constitutive | Altered | Decreased ABA sensitivity |
| | | thaliana | | 35S | hormone | |
| | | | | | sensitivity | |
| G2992 | 286 | Arabidopsis | G2999 | Constitutive | Altered | Decreased ABA sensitivity |
| | | thaliana | | 35S | hormone | |
| | | | | | sensitivity | |
| G2995 | 288 | Arabidopsis | G2999 | Constitutive | Altered | Decreased ABA sensitivity |
| | | thaliana | | 35S | hormone | |
| | | | | | sensitivity | |
| G2999 | 256 | Arabidopsis | G2999 | Leaf RBCS3 | Altered | Decreased ABA sensitivity |
| | | thaliana | | | hormone | |
| | | | | | sensitivity | |
| G3685 | 274 | Oryza sativa | G2999 | Constitutive | Altered | Decreased ABA sensitivity |
| | | | | 35S | hormone | |
| | | | | | sensitivity | |
| G2995 | 288 | Arabidopsis | G2999 | Constitutive | Tolerance to | Increased tolerance to |
| | | thaliana | | 35S | hyperosmotic | mannitol |
| | | | | | stress | |
| G3690 | 262 | Oryza sativa | G2999 | Constitutive | Tolerance to | Increased tolerance to |
| | | | | 35S | hyperosmotic | mannitol |
| | | | | | stress | |
| G2999 | 256 | Arabidopsis | G2999 | Leaf RBCS3 | Tolerance to | Increased tolerance to sucrose |
| | | thaliana | | | hyperosmotic | |
| | | | | | stress | |
| G2995 | 288 | Arabidopsis | G2999 | Constitutive | Tolerance to | Increased tolerance to sucrose |
| | | thaliana | | 35S | hyperosmotic | |
| | | | | | stress | |
| G2996 | 270 | Arabidopsis | G2999 | Leaf RBCS3 | Tolerance to | Increased tolerance to sucrose |
| | | thaliana | | | hyperosmotic | |
| | | | | | stress | |
| G2991 | 282 | Arabidopsis | G2999 | Constitutive | Altered root | Increased root mass |
| | | thaliana | | 35S | morphology | |
| G2995 | 288 | Arabidopsis | G2999 | Constitutive | Tolerance to | Increased tolerance to NaCl |
| | | thaliana | | 35S | sodium | |
| | | | | | chloride | |
| G3676 | 266 | Zea mays | G2999 | Constitutive | Tolerance to | Increased tolerance to NaCl |
| | | | | 35S | sodium | |
| | | | | | chloride | |
| G3681 | 278 | Zea mays | G2999 | Constitutive | Tolerance to | Increased tolerance to NaCl |
| | | | | 35S | sodium | |
| | | | | | chloride | |
| G3760 | 324 | Zea mays | G3086 | Constitutive | Tolerance to | Increased tolerance to NaCl |
| | | | | 35S | sodium | |
| | | | | | chloride | |
| G2555 | 318 | Arabidopsis | G3086 | Constitutive | Heat tolerance | Increased tolerance to heat |
| | | thaliana | | 35S | | |
| G3750 | 326 | Oryza sativa | G3086 | Constitutive | Heat tolerance | Increased tolerance to heat |
| | | | | 35S | | |
| G2555 | 318 | Arabidopsis | G3086 | Constitutive | Cold tolerance | Increased tolerance to cold |
| | | thaliana | | 35S | | |
| G2766 | 322 | Arabidopsis | G3086 | Constitutive | Cold tolerance | Increased tolerance to cold |
| | | thaliana | | 35S | | |
| G3086 | 292 | Arabidopsis | G3086 | Constitutive | Cold tolerance | Increased tolerance to cold |
| | | thaliana | | 35S | | |
| G3755 | 302 | Zea mays | G3086 | Constitutive | Cold tolerance | Increased tolerance to cold |
| | | | | 35S | | |
| G3760 | 324 | Zea mays | G3086 | Constitutive | Cold tolerance | Increased tolerance to cold |
| | | | | 35S | | |
| G3766 | 304 | Glycine max | G3086 | Constitutive | Cold tolerance | Increased tolerance to cold |
| | | | | 35S | | |
| G3086 | 292 | Arabidopsis | G3086 | Double Over- | Altered | Early flowering |
| | | thaliana | | expression | flowering time | |
| | | | | (with G481) | | |
| G3086 | 292 | Arabidopsis | G3086 | KO | Altered | Late flowering |
| | | thaliana | | | flowering time | |
| G3086 | 292 | Arabidopsis | G3086 | RSI1 | Altered | Early flowering |
| | | thaliana | | | flowering time | |
| G3760 | 324 | Zea mays | G3086 | Constitutive | Altered | Early flowering |
| | | | | 35S | flowering time | |
| G3086 | 292 | Arabidopsis | G3086 | Constitutive | Drought | Increased tolerance to |
| | | thaliana | | 35S | tolerance | dehydration |
| G3750 | 326 | Oryza sativa | G3086 | Constitutive | Drought | Increased tolerance to |
| | | | | 35S | tolerance | dehydration |
| G3750 | 326 | Oryza sativa | G3086 | Constitutive | Drought | Increased tolerance to |
| | | | | 35S | tolerance | dehydration |
| G3765 | 314 | Glycine max | G3086 | Constitutive | Drought | Increased tolerance to drought |
| | | | | 35S | tolerance | in soil assays |
| G3767 | 298 | Glycine max | G3086 | Constitutive | Drought | Increased tolerance to |
| | | | | 35S | tolerance | dehydration |
| G3769 | 296 | Glycine max | G3086 | Constitutive | Drought | Increased tolerance to |
| | | | | 35S | tolerance | dehydration |
| G3771 | 312 | Glycine max | G3086 | Constitutive | Drought | Increased tolerance to |
| | | | | 35S | tolerance | dehydration |
| G3771 | 312 | Glycine max | G3086 | Constitutive | Drought | Increased tolerance to drought |
| | | | | 35S | tolerance | in soil assays |
| G3766 | 304 | Glycine max | G3086 | Constitutive | Drought | Increased tolerance to |
| | | | | 35S | tolerance | dehydration |
| G1134 | 316 | Arabidopsis | G3086 | Constitutive | Altered | Decreased ABA sensitivity |
| | | thaliana | | 35S | hormone | |
| | | | | | sensitivity | |
| G3744 | 300 | Oryza sativa | G3086 | Constitutive | Altered | Decreased ABA sensitivity |
| | | | | 35S | hormone | |
| | | | | | sensitivity | |
| G3750 | 326 | Oryza sativa | G3086 | Constitutive | Altered | Decreased ABA sensitivity |
| | | | | 35S | hormone | |
| | | | | | sensitivity | |
| G3760 | 324 | Zea mays | G3086 | Constitutive | Altered | Decreased ABA sensitivity |
| | | | | 35S | hormone | |
| | | | | | sensitivity | |
| G3765 | 314 | Glycine max | G3086 | Constitutive | Altered | Decreased ABA sensitivity |
| | | | | 35S | hormone | |
| | | | | | sensitivity | |
| G3766 | 304 | Glycine max | G3086 | Constitutive | Altered | Decreased ABA sensitivity |
| | | | | 35S | hormone | |
| | | | | | sensitivity | |
| G3767 | 298 | Glycine max | G3086 | Constitutive | Altered | Decreased ABA sensitivity |
| | | | | 35S | hormone | |
| | | | | | sensitivity | |
| G3768 | 294 | Glycine max | G3086 | Constitutive | Altered | Decreased ABA sensitivity |
| | | | | 35S | hormone | |
| | | | | | sensitivity | |
| G3769 | 296 | Glycine max | G3086 | Constitutive | Altered | Decreased ABA sensitivity |
| | | | | 35S | hormone | |
| | | | | | sensitivity | |
| G3766 | 304 | Glycine max | G3086 | Constitutive | Altered | Early flowering |
| | | | | 35S | flowering time | |
| G3767 | 298 | Glycine max | G3086 | Constitutive | Altered | Early flowering |
| | | | | 35S | flowering time | |
| G3768 | 294 | Glycine max | G3086 | Constitutive | Altered | Early flowering |
| | | | | 35S | flowering time | |
| G3769 | 296 | Glycine max | G3086 | Constitutive | Altered | Early flowering |
| | | | | 35S | flowering time | |
| G3771 | 312 | Glycine max | G3086 | Constitutive | Altered | Early flowering |
| | | | | 35S | flowering time | |
| G3744 | 300 | Oryza sativa | G3086 | Constitutive | Tolerance to | Increased tolerance to sucrose |
| | | | | 35S | hyperosmotic | |
| | | | | | stress | |

In this Example, unless otherwise indicted, morphological and physiological traits are disclosed in comparison to wild-type control plants. That is, a transformed plant that is described as large and/or drought tolerant is large and more tolerant to drought with respect to a wild-type control plant. When a plant is said to have a better performance than controls, it generally showed less stress symptoms than control plants. The better performing lines may, for example, produce less anthocyanin, or be larger, green, or more vigorous in response to a particular stress, as noted below. Better performance generally implies greater tolerance to a particular biotic or abiotic stress, less sensitivity to ABA, or better recovery from a stress (as in the case of a drought treatment) than controls.

### The G28 Clade

G1006 (SEQ ID NO: 151 and 152; Arabidopsis thaliana)—Constitutive 35S

Background. G1006 is a closely-related Arabidopsis homolog of G28. It has been described in the public literature as AtERF2, and has been demonstrated to be induced by ethylene, methyl jasmonate, and pathogens (Fujimoto et al., 2000; Chen et al., 2002; Brown et al., 2003).

Morphological Observations. G1006 produced dwarfing when overexpressed. Almost all of the lines in each of two different batches were small and slow developing, although in one batch dwarfing was not initially evident. They also typically exhibited dark, shiny leaves.

Disease Assay Results. Eight 35S::G1006 lines were tested by Sclerotinia plate assay. Four of these lines (305, 308, 315, and 320) showed a small degree of enhanced resistance to Sclerotinia infection.

TABLE 26
G1006 disease assay results:

| | | Project | | | |
|---|---|---|---|---|---|
| Line | PID | Type | Botrytis | Sclerotinia | Erysiphe |
| 302 | P417 | DPF | n/d | wt | n/d |
| 304 | P417 | DPF | n/d | wt | n/d |
| 305 | P417 | DPF | n/d | + | n/d |
| 308 | P417 | DPF | n/d | + | n/d |
| 314 | P417 | DPF | n/d | wt | n/d |
| 315 | P417 | DPF | n/d | + | n/d |
| 318 | P417 | DPF | n/d | wt | n/d |
| 320 | P417 | DPF | n/d | + | n/d |

DPF = direct promoter fusion
n/d = not determined

Discussion. Lines overexpressing G1006 were generally smaller and slower developing than controls, and had dark green, shiny leaves. These morphological phenotypes were similar to those observed in G28 lines with moderate to high expression levels, and to those observed in a number of G28 orthologs. Four out of eight lines tested showed some degree of enhanced resistance to Sclerotinia infection, indicating that G1006 functions similarly to G28 in disease resistance. Resistance to Botrytis cinerea and powdery mildew have not yet been tested.

Potential applications. G1006 may be useful for engineering pathogen resistance in crop plants.

G3430 (SEQ ID NO: 167 and 168; Oryza sativa)—Constitutive 35S

Background. G3430 is a rice ortholog of G28. The aim of this project was to determine whether overexpression of G3430 in Arabidopsis produces comparable effects to those of G28 overexpression.

Morphological Observations. In the first batch of plants, all eleven lines had wavy leaves in the vegetative phase. All lines were also smaller and darker green, when compared to controls (except line 304 which was marginally large). All lines in this first batch also had shiny leaves and were late developing.

The second batch of 35S::G3430 plants showed no consistent differences to controls. An exception was line 322, which was small and dark green and died prior to bolting. All plants showed slightly wavy leaves.

Disease Assay Results. Ten 35S::G3430 lines were tested by Sclerotinia plate assay. Five lines (306, 308, 328, and 330) displayed increased resistance to this pathogen. Seven of 10 lines were moderately to highly resistant to Erysiphe in a soil-based assay as compared to controls.

TABLE 27
35S::G3430 disease assay results:

| | | Project | | | |
|---|---|---|---|---|---|
| Line | PID | Type | Botrytis | Sclerotinia | Erysiphe |
| 306 | P21267 | DPF | + | + | +++ |
| 308 | P21267 | DPF | wt | + | +++ |
| 321 | P21267 | DPF | + | wt | +++ |
| 323 | P21267 | DPF | wt | wt | wt |
| 324 | P21267 | DPF | wt | wt | ++ |
| 325 | P21267 | DPF | wt | + | wt |
| 326 | P21267 | DPF | wt | wt | +++ |
| 327 | P21267 | DPF | wt | wt | wt |
| 328 | P21267 | DPF | wt | + | ++ |
| 330 | P21267 | DPF | wt | + | +++ |

DPF = direct promoter fusion
n/d = not determined

Discussion. Overexpression of G3430 produced inconsistent effects on Arabidopsis morphology. One batch of 35S::G3430 lines was small, dark green, and late developing, while a second batch was not significantly different from controls. High expressing 35S::G28 plants and most G28 orthologs tested show a small, dark green, late flowering phenotype, suggesting that the phenotype seen in the first set of transgenic plants is accurate. The expression of this phenotype may vary depending on growth conditions.

Ten 35S::G3430 lines have been tested in disease assays to date. Five of these lines showed enhanced resistance to Sclerotinia in a plate assay; seven of these lines showed enhanced resistance to Erysiphe, and two of the lines resistant to Erysiphe were also resistant to Botrytis. Several lines are resistant to multiple pathogens. The lines tested came from both batches of T1 plants.

Potential applications. G3430 may be used to increase disease resistance or modify flowering time in plants.

G3659 (SEQ ID NO: 149 and 150; Brassica oleracea)—Constitutive 35S

Background. G3659 was included in the disease lead advancement program as a Brassica oleracea ortholog of G28. The aim of this project was to determine whether overexpression of G3659 in Arabidopsis produces comparable effects to those of G28 overexpression.

Morphological Observations. Overexpression of G3659 produced plants that were small, dark green and late developing.

Disease Assay Results. Of the 35S::G3659 lines tested in a plate assay for Sclerotinia resistance; two lines showed some degree of resistance. Four lines tested in an Erysiphe soil assay were more resistant to this pathogen than controls, with the level of resistance ranging from somewhat to highly resistant.

TABLE 28
35S::G3659 Disease assay results:

| | | Project | | | |
|---|---|---|---|---|---|
| Line | PID | Type | Botrytis | Sclerotinia | Erysiphe |
| 301 | P23452 | DPF | wt | wt | wt |
| 303 | P23452 | DPF | n/d | wt | wt |
| 305 | P23452 | DPF | wt | wt | ++ |
| 306 | P23452 | DPF | + | wt | + |
| 308 | P23452 | DPF | wt | + | +++ |
| 310 | P23452 | DPF | n/d | + | +++ |
| 311 | P23452 | DPF | n/d | wt | wt |
| 315 | P23452 | DPF | n/d | wt | wt |
| 316 | P23452 | DPF | n/d | wt | wt |
| 317 | P23452 | DPF | n/d | wt | wt |

DPF = direct promoter fusion
n/d = not determined

Discussion. Overexpression of G3659 produced plants that were small, dark green and late developing. These morphological effects are similar to the phenotype observed in high expressing 35S::G28 lines and plants expressing most G28 orthologs. Ten 35S::G3659 lines have been tested in a plate assay for Sclerotinia resistance, with two lines showing some degree of resistance. Of the ten 35S::G3659 lines that have been tested in a plate assay for Sclerotinia resistance, three lines showed greater resistance than controls.

Potential applications. Based on the results obtained so far, G3659 may be used to increase disease resistance or modify flowering time in plants.

G3660 (SEQ ID NO: 157 and 158. Brassica oleracea)—Constitutive 35S

Background. G3660 is a Brassica oleracea ortholog of G1006, a closely-related Arabidopsis homolog of G28. The aim of this project was to determine whether overexpression of G3660 in Arabidopsis produces comparable effects to those of G28 overexpression.

Morphological Observations. The overexpression of G3660 consistently induced dwarfing in Arabidopsis. Seventy-five percent of the T1 transformants were noticeably small at seven days. All lines isolated were small, dark green and had shiny leaves. Plant size and flowering time were variable, although all lines were small and flowered late to some degree.

Disease Assay Results. Ten 35S::G3660 lines were tested by Sclerotinia plate assay. Three lines (302, 308, and 340) showed a degree of enhanced resistance to Sclerotinia infection. Ten lines were tested in an Erysiphe soil assay, and all lines tested were moderately to highly resistant to this pathogen.

TABLE 29
35S::G3660 Disease assay results:

| | | Project | | | |
|---|---|---|---|---|---|
| Line | PID | Type | Botrytis | Sclerotinia | Erysiphe |
| 301 | P23418 | DPF | n/d | wt | +++ |
| 302 | P23418 | DPF | wt | + | +++ |
| 305 | P23418 | DPF | n/d | wt | ++ |
| 307 | P23418 | DPF | n/d | wt | +++ |
| 308 | P23418 | DPF | + | + | +++ |
| 321 | P23418 | DPF | n/d | wt | +++ |
| 324 | P23418 | DPF | n/d | wt | +++ |
| 327 | P23418 | DPF | n/d | wt | +++ |
| 330 | P23418 | DPF | n/d | wt | + |
| 340 | P23418 | DPF | + | + | +++ |

DPF = direct promoter fusion
n/d = not determined

Discussion. Overexpression of G3660 produced plants that were small, dark green and late developing. These morphological effects were similar to the phenotype observed in high expressing 35S::G28 plants and plants expressing most closely-related G28 homologs. Ten 35S::G3660 lines have been tested in a plate assay for Sclerotinia resistance; three lines showed greater resistance than controls. In a soil based assay, all ten lines tested were more resistant to Erysiphe than controls.

Potential applications. Based on the results obtained so far, G3660 may be used to increase disease resistance or modify flowering time in plants.

G3661 (SEQ ID NO: 161 and 162; Zea mays)—Constitutive 35S

Background. G3661 is maize ortholog of G28. The aim of this project is to determine whether overexpression of G3661 in Arabidopsis produces comparable effects to those of G28 overexpression.

Morphological Observations. Overexpression of G3661 produced plants that were small, dark green and late developing.

Disease Array Results. Five of 10 lines showed evidence of greater tolerance to Erysiphe than controls in plate-based assays, including two lines that were highly resistant.

TABLE 30
35S::G3661 Disease assay results:

| | | Project | | | |
|---|---|---|---|---|---|
| Line | PID | Type | Botrytis | Sclerotinia | Erysiphe |
| 301 | P23419 | DPF | n/d | wt | wt |
| 302 | P23419 | DPF | n/d | − | + |
| 306 | P23419 | DPF | n/d | wt | +++ |
| 308 | P23419 | DPF | n/d | wt | + |
| 311 | P23419 | DPF | n/d | wt | + |
| 312 | P23419 | DPF | n/d | wt | +++ |
| 314 | P23419 | DPF | n/d | wt | n/d |
| 321 | P23419 | DPF | n/d | − | n/d |
| 322 | P23419 | DPF | n/d | wt | n/d |
| 323 | P23419 | DPF | n/d | wt | wt |

DPF = direct promoter fusion
n/d = not determined

Discussion. The morphological effects conferred by G3661 are similar to the phenotype observed in high expressing 35S::G28 plants and plants expressing most closely-related G28 homologs. While some of these overexpressors were much more resistant to Erysiphe than controls, two lines were somewhat more sensitive to Sclerotinia. A screening step to eliminate those plants that are more sensitive to the latter pathogen than controls would likely be advantageous.

Potential applications. Based on the results obtained so far, G3661 may be used to increase Erysiphe resistance or modify flowering time in plants.

G3717 (SEQ ID NO: 153 and 154; Glycine max)—Constitutive 35S

Background. G3717 was included in the disease lead advancement program as a soy ortholog of G28. The aim of this project was to determine whether overexpression of G3717 in Arabidopsis produces comparable effects to those of G28 overexpression.

Morphological Observations. Overexpression of G3717 produced plants that were severely dwarfed, dark green, and late developing. A small, dark green, late developing phenotype is common among plants expressing members of the G28 clade, but the effects of G3717 are particularly severe.

Disease Assay Results. In a soil based assay, 35S::G3717 lines were found to be generally more resistant than wild-type controls to Erysiphe. For five of the lines tested, the level of resistance conferred as compared to controls was highly significant.

TABLE 31
35S::G3717 Disease assay results:

| | | Project | | | |
|---|---|---|---|---|---|
| Line | PID | Type | Botrytis | Sclerotinia | Erysiphe |
| 321 | P23421 | DPF | n/d | n/d | +++ |
| 323 | P23421 | DPF | n/d | wt | +++ |
| 343 | P23421 | DPF | n/d | n/d | +++ |
| 344 | P23421 | DPF | n/d | n/d | +++ |
| 345 | P23421 | DPF | n/d | wt | + |
| 346 | P23421 | DPF | n/d | n/d | wt |
| 365 | P23421 | DPF | n/d | n/d | +++ |
| 367 | P23421 | DPF | n/d | n/d | ++ |

DPF = direct promoter fusion
n/d = not determined

Discussion. Due to these deleterious effects on growth, the disease resistance conferred by G3717 may be best utilized if expression of this gene is optimized with a judicious regulatory mechanism.

Potential applications. Based on the results obtained so far, G3717 may be used to increase disease resistance in plants.

G3718 (SEQ ID NO: 155 and 156; Glycine max)—Constitutive 35S

Background. G3718 was included in the disease lead advancement program as a soy ortholog of G28. The aim of this project was to determine whether overexpression of G3718 in Arabidopsis produces comparable effects to those of G28 overexpression.

Morphological Observations. Overexpression of G3718 produced plants that were small, dark green and late developing.

Disease Assay Results. Of the 35S::G3718 lines tested in a plate assay for Sclerotinia resistance; two lines showed some degree of resistance. Five 35S::G3718 lines were more resistant than wild type in soil-based assays.

TABLE 32
35S::G3718 Disease assay results:

| | | Project | | | |
|---|---|---|---|---|---|
| Line | PID | Type | Botrytis | Sclerotinia | Erysiphe |
| 302 | P23423 | DPF | n/d | wt | +++ |
| 303 | P23423 | DPF | n/d | wt | wt |
| 304 | P23423 | DPF | n/d | wt | wt |
| 305 | P23423 | DPF | n/d | wt | wt |
| 306 | P23423 | DPF | wt | + | wt |
| 307 | P23423 | DPF | wt | wt | ++ |
| 308 | P23423 | DPF | n/d | wt | ++ |
| 309 | P23423 | DPF | n/d | wt | +++ |
| 313 | P23423 | DPF | + | + | n/d |
| 324 | P23423 | DPF | n/d | n/d | +++ |

DPF = direct promoter fusion
n/d = not determined

Discussion. The morphological effects conferred by G3718 were similar to the phenotype observed in high expressing 35S::G28 lines and plants expressing most closely-related G28 homologs. Due to these deleterious effects on growth, the disease resistance conferred by G3718 may be best utilized if expression of this gene is optimized with a judicious regulatory mechanism.

Potential applications. Based on the results obtained so far, G3718 may be used to increase disease resistance or modify flowering time in plants.

### The G47 Clade

G2133 (SEQ ID NO: 175 and 176; Arabidopsis thaliana)—Constitutive 35S

Two new sets of 35S::G2133 direct promoter fusion lines have been obtained. In both cases, the majority of lines were markedly dwarfed at early stages and exhibited vertically oriented leaves. Many of the lines were late flowering, and at late stages a significant number of lines showed fleshy, succulent, leaves and stems and reduced apical dominance. These effects were similar to those seen in 35S::G47 lines.

Morphological Observations. Many of the 35S::G2133 lines at early stages were small. Some lines flowered late and showed upright leaves. A few other lines had vitreous inner rosette leaves. Several lines showed fleshy leaves and sterns and had a reduction in apical dominance at late stages. Four lines were phenotypically similar to wild-type.

Physiology (Plate assays) Results. Five out of ten 35S::G2133 lines were more tolerant to cold in a germination assay. Five lines were also more tolerant to a water deprivation stress, as shown in a severe dehydration plate-based assay.

Physiology (Soil Drought-Clay Pot) Summary. Numerous independent 35S::G2133 lines have been tested in soil drought assays and each showed more tolerance to and/or better recovery from drought conditions than controls.

TABLE 33
35S::G2133 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 3 | DPF | 5.3 | 0.71 | 0.000018* | 0.7 | 0.17 | 0.00000000000000000033* |
| 4 | DPF | 4.2 | 0.71 | 0.0000071* | 0.54 | 0.17 | 0.0000000000000051* |
| 5 | DPF | 6 | 0.71 | 0.00051* | 0.88 | 0.17 | 0.00000000000049* |
| 311 | DPF | 1.5 | 0.7 | 0.04* | 0.26 | 0.1 | 0.00057* |
| 311 | DPF | 1 | 0.3 | 0.022* | 0.16 | 0.05 | 0.0051* |
| 312 | DPF | 1.4 | 0.9 | 0.27 | 0.21 | 0.16 | 0.22 |
| 312 | DPF | 1.6 | 0.6 | 0.033* | 0.21 | 0.093 | 0.006* |
| 316 | DPF | 1.6 | 0.1 | 0.00051* | 0.29 | 0.036 | 0.0000011* |
| 316 | DPF | 1.4 | 0.5 | 0.022* | 0.17 | 0.071 | 0.018* |
| MIXED | DPF | 1.6 | 0.9 | 0.18 | 0.25 | 0.14 | 0.017* |

DPF = direct promoter fusion
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. As expected, 35S::G2133 overexpressing lines were dwarfed, had fleshy leaves and stems, and reduced apical dominance later in development. G2133 overexpressors showed improved cold germination and greater tolerance to water deprivation as compared to controls in plates and soil-based drought assays.

Potential applications. G2133 provides enhanced cold germination and drought tolerance. The gene might also be used to modify developmental traits such as flowering time and inflorescence architecture.

G2133 (SEQ ID NO: 175 and 176; Arabidopsis thaliana)—Leaf RBCS3

Background. G2133 is a very closely related homolog of G47, having 65% sequence identity over the entire length of the protein (138 amino acids), and had previously shown excellent drought tolerance in soil assays. These proteins presumably have diverged relatively recently in Arabidopsis. The objective of this project was to determine whether leaf mesophyll-specific expression of G2133 would separate the stress tolerance and morphological phenotypes.

Morphological Observations. Twenty RBCS3::G2133 lines have been generated using a two-component approach. Considerable size variation was seen among these T1 plants, but overall, no consistent differences to controls were noted. The severe dwarfing that is apparent in 35S::G2133 lines was not seen.

Physiology (Plate assays) Results. RBCS3::G2133 lines were more tolerant relative to wild-type in a severe plate-based dehydration assay (three lines out of ten), and in a growth assay under chilling conditions (six lines out of ten).

Discussion. Two-component RBCS3::G2133 T1 lines have been found to have significantly varied plant size, but no consistent differences with control were observed. Thus, dwarfing and the fleshy leaves and stems observed with 35S ectopic expression were eliminated using the RBCS3 promoter. RBCS3::G2133 overexpressing lines also showed tolerance to severe dehydration stress and improved growth in cold conditions in plate assays. Thus, stress tolerance was retained with elimination of significant morphological abnormalities. It should also be noted that we obtained cold-stress tolerance with the RBCS3 lines for the related gene, G47.

G2133 (SEQ ID NO: 175 and 176; Arabidopsis thaliana)—Stress Inducible RD29A—Line 5

Background. The objective of this project was to determine whether drought-inducible expression of G2133 would support stress tolerance without adverse morphological phenotypes.

Morphological Observations. RD29A::G2133 two-component lines plants were noted to be marginally smaller than wild-type at the rosette stage, but otherwise appeared wild type.

Physiology (Plate assays) Results. Three out of ten RD29A::G2133 lines were more tolerant to a severe plate based dehydration stress compared to wild-type control seedlings.

Physiology (Soil Drought-Clay Pot) Summary. The three lines that were more tolerant to dehydration stress were tested in soil based assays and one of these showed a repeatedly better survival than controls across two different plantings. Importantly, the T2 lines tested in soil drought assays showed no clear developmental abnormalities or size reductions.

Discussion. Two-component RD29A (line 5)::G2133 overexpressors lines are marginally smaller than control plants at the rosette stage, but otherwise are unaltered in growth and development. Three lines exhibited increased water deprivation tolerance (desiccation tolerance in the plate-based extreme dehydration assay), one of these lines showed improved drought tolerance in a soil assay, and the T2 lines showed no developmental abnormalities or size reductions

Potential applications. RD29A::G2133 overexpression provides enhanced cold germination and water deprivation tolerance with few or no adverse morphological or developmental defects. The gene might also be used to modify developmental traits such as flowering time and inflorescence architecture.

G47 (SEQ ID NO: 173 and 174; Arabidopsis thaliana)—Leaf RBCS3

Background. G47 was included in the drought program based on the enhanced vegetative yield and the drought tolerance shown by 35S::G47 lines.

The objective of this project was to determine whether leaf mesophyll-specific expression of G47 would separate the stress tolerance and morphological phenotypes characteristic of G47 overexpression.

Morphological Observations. RBCS3::G47 lines have been generated using a two-component approach, and three different batches of T1 lines have been obtained. Some of these seedlings were slightly small at early stages, but severe dwarfing effects were not observed. Some lines were late flowering. A considerable number of lines showed no consistent differences in morphology to controls.

Physiology (Plate assays) Results. Six of 10 RBCS3::G47 lines were more tolerant to salt than wild-type controls in germination assays. RBCS3::G47 overexpressors were also observed to be more tolerant to hyperosmotic stress; 3 of 10 lines were more tolerant to mannitol and 3 of 10 lines were more tolerant to sucrose than controls. Six of 10 lines showed better germination in the cold than wild-type in plate assays. Overall, stress tolerance similar to that observed with 35S::G47 lines was retained with elimination of significant morphological abnormalities. It should also be noted that we obtained cold-stress and hyperosmotic stress (desiccation) tolerance with the RBCS3 lines for the related gene, G2133.

Discussion. Two-component RBCS3::G47 lines have been found to have nearly normal growth and development compared with controls. Some lines were slightly small early in development, and some lines showed a late flowering phenotype. However, the severe dwarfing, and leaf and stem thickness, seen with 35S::G47 lines, were not observed.

Potential applications. G47 provides enhanced cold and drought tolerance. The utility of leaf mesophyll expression for stress tolerance is promising. The RBCS3::G47 combination might also be useful for manipulating flowering time.

G47 (SEQ ID NO: 173 and 174; Arabidopsis thaliana)—Stress Inducible RD29A—Line 5

Background. The objective of this project was to determine whether drought-inducible expression of G47 via the RD29A promoter would support stress tolerance without adverse morphological phenotypes.

Morphological observations. Two-component RD29A::G47 lines were somewhat dwarfed early in development, although significant size variation was observed. Some lines were later flowering.

Physiology (Plate assays) Results. Half of the lines (5 of 10) performed better than controls in a water deprivation, severe dehydration plate assay, and 3 of 10 lines were insensitive to ABA in a germination assay. This contrasts with the 35S::G47 (direct fusion) lines which showed little increased stress tolerance in plate assays.

Physiology (Soil Drought-Clay Pot) Summary. One RD29A::G47 line of three lines examined exhibited significantly better recovery than control plants is a water deprivation, soil-based drought assay.

Potential applications. At this stage of the analysis, we have shown that drought-inducible expression of G47 can significantly ameliorate growth abnormalities observed using the 35S promoter, and that some stress tolerance is retained. The RD29A::G47 lines were not completely wild-type, which might be attributed to G47 expression from the RD29A promoter in embryos and young seedlings.

G47 (SEQ ID NO: 173 and 174; Arabidopsis thaliana)—Super Activation (N-GAL4-TA)

Background. The aim of this study was to determine whether addition of a strong transcription activation domain from the yeast GAL4 gene could enhance potency of the 35S::G47 phenotype.

Morphological Observations. 35S::N-GAL4-G47 T1 lines showed a mild acceleration in the onset of flowering, relative to wild-type controls. In other regards, these lines appeared wild type.

Physiology Summary. In assays performed thus far, nine of ten 35S::N-GAL4-G47 lines tested were more tolerant to mannitol than wild-type control plants, indicating a hyperosmotic stress tolerant phenotype.

Discussion. In contrast to 35S::G47 lines, which had multiple developmental alterations including delayed flowering, the 35S::N-GAL4-TA-G47 lines flowered earlier than controls. In other aspects of development, these lines appeared similar to wild type.

Potential applications. At this stage of the analysis, the N-terminal GAL4 fusion was found to mitigate undesirable morphological changes, but we have not determined the utility of an N-terminal fusion for drought tolerance. Based on the morphological effects observed, the GAL-G47 fusion can be used to modify flowering time. The mannitol tolerance results indicate that the GAL-G47 fusion may be used to confer drought and drought-related stress tolerance in plants.

G47 (SEQ ID NO: 173 and 174; Arabidopsis thaliana)—Vascular SUC2

Background. The objective of this project was to determine whether phloem companion cell-specific expression of G47 would separate the stress tolerance and morphological phenotypes observed with 35S::G47 lines.

Morphological Observations. SUC2::G47 two-component system transformants were mostly wild-type at early stages, but a significant number of the lines were late flowering and exhibited rather dark leaves versus controls. A number of lines were late flowering and/or showed dark leaves. The stems from these plants were also potentially thicker (detailed measurements were not taken) than those of controls.

Physiology Results. Three of 10 SUC2::G47 lines were more tolerant than wild type controls in severe desiccation assays.

Discussion: Two-component SUC2::G47 lines displayed wild-type growth at early stages, but many lines were delayed in flowering. Later in development, enlarged and greener leaves were observed. The stems of some of these lines may also be somewhat thicker than stems of controls. Thus, expression using the SUC2 promoter eliminates the significant dwarfing effects associated with constitutive overexpression.

Potential applications. G47 provides enhanced drought tolerance. The utility of phloem companion cell-specific expression for these traits remains to be determined. The morphological phenotypes seen in SUC2::G47 lines indicate that this combination can be useful for modifying developmental traits such as flowering time, leaf size, stem structure, and coloration.

G47 (SEQ ID NO: 173 and 174; Arabidopsis thaliana)—Shoot Apical Meristem STM

Background. The objective of this study was to determine whether the morphological and stress phenotypes associated with 35S::G47 overexpression could be resolved with meristem-specific expression using the STM promoter.

Morphological Observations. Two sets of STM::G47 lines have been obtained using the two-component system. Lines 1001-1020 isolated in one STM driver line showed wild-type morphology at all developmental stages. Lines isolated in another STM driver line were small at early stages with a number of the lines showing delayed flowering. At late stages some of the second STM driver lines exhibited larger rosettes than wild type.

Physiology (Plate assays) Results. In assays performed thus far, 5 of 10 STM::G47 lines were more tolerant to sucrose, and 7 of 10 lines were more tolerant to germination in cold conditions, than wild-type control plants.

Discussion. STM::G47 overexpression via the two-component system in one driver line yielded plants with growth and development comparable to that of controls. G47 overexpression with a second driver line yielded plants that were generally small early in development, but some of which flowered rather late and developed enlarged leaves at late stages.

Potential applications. We have shown that meristem-specific expression of G47 can result in normal plants, but have yet to determine whether drought tolerance is retained with this expression pattern. However, it is possible that expression of G47 at high levels in meristems can be useful for modifying developmental traits such as flowering time and leaf size.

G2115 (SEQ ID NO: 405 and 406; Arabidopsis thaliana)—Constitutive 35S

Background. G2115 was included in the G47 study group as an outlier to help define the specific structural motifs necessary for abiotic stress tolerance and drought tolerance. G2115 lies in a closely-related clade of AP2 transcription factors. The few 35S::G2115 lines tested previously in the earlier genomics program did not show the fleshy stems characteristic of G47 overexpression, and were not found to confer stress-tolerance.

Morphological Observations. G2115 overexpressing lines showed deleterious effects on morphology; all were dwarfed to varying extents and a number of lines were early flowering. Other lines were slow developing, bolted late, and exhibited various non-specific floral abnormalities.

Physiology (Plate assays) Results. Four of 10 lines were observed to be more tolerant to cold stress in a germination assay, and 3 of 10 lines were more tolerant than wild-type controls in a cold growth assay.

Discussion: As was observed in the genomics program, 35S::G2115 overexpressing lines were somewhat dwarfed and showed a variety of morphological defects. Some lines flowered earlier than controls, while other lines flowered later. G2115 overexpressing lines showed improved germination and growth in the cold in plate based assays. At the time of this these lines have not been evaluated for drought tolerance in a soil assay.

Potential applications: G2115 provides enhanced germination and growth under cold conditions. Given the deleterious effects on development, the gene might require optimization with tissue specific or inducible promoters.

G3643 (SEQ ID NO: 177 and 178; Glycine max)—Constitutive 35S

Background. G3643 was included as a soybean ortholog of G47. The objective of this project was to determine whether G3643 can condition drought tolerance when expressed in Arabidopsis.

Morphological Observations. All 35S::G3643 lines were small, particularly at early stages, and a substantial number of lines exhibited delayed flowering. At late stages, several lines were noted to be slightly larger than controls. Overall, the fleshy stem and leaf phenotype was less marked in this set of lines than in 35S::G47 lines.

Physiology (Plate assays) Results. Three of 10 G3643 overexpressing lines were more tolerant than wild type to cold in a germination assay.

Physiology (Soil Drought-Clay Pot) Summary. Three independent 35S::G3643 lines have recently been tested in a soil drought assay and each showed more tolerance to and better recovery from drought conditions than controls.

TABLE 34
35S::G3643 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 305 | DPF | 1.9 | 1.4 | 0.26 | 0.56 | 0.39 | 0.0043* |
| 307 | DPF | 1.3 | 0.30 | 0.059* | 0.34 | 0.057 | 0.00000017* |
| 313 | DPF | 1.0 | 0 | 0.0058* | 0.23 | 0.0071 | 0.00028* |

DPF = direct promoter fusion
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3643 direct fusion lines were smaller than controls, with a number of lines having delayed flowering compared to controls. Overall, the fleshy stem and leaf phenotype was less marked in this set of lines than in 35S::G47 lines. Improved cold germination was observed in plate assays, and two lines with enhanced cold germination also were more tolerant than controls in soil drought assays.

Potential applications. Based on the results from stress assays, G3643 provides enhanced cold germination and drought tolerance. However, given the developmental effects of overexpression of this gene, it might require optimization with a tissue specific or inducible promoter.

G3644 (SEQ ID NO: 181 and 182; Oryza sativa)—Constitutive 35S

Background. G3644 is a rice ortholog of G47. The aim of this project was to determine whether overexpression of G3644 produces comparable effects on morphology and stress tolerance to overexpression of G47.

Morphological Observations. Dwarfing was apparent in the 35S::G3644 lines at early stages. Many individuals showed large leaves and thick stems that were somewhat reminiscent of those in 35S::G47 lines. However, these features were not apparent in the rest of the lines. Four lines were slightly late flowering. Two lines were noted to be bushy at late stages. In some lines, considerable size variation was apparent early in development, with three lines being particularly small. Later, most of the lines developed enlarged rosettes and rather thick stems.

Discussion. Direct fusion 35S::G3644 lines had morphological phenotypes similar to 35S::G47 lines: early dwarfing was observed in most lines, and some lines also exhibited thick leaves and stems. Thus, the G3644 proteins shares some activity with G47. No stress tolerance was observed in any plate assay, and surprisingly, the G3644 overexpressing lines were consistently more sensitive to cold than the controls. This was particularly intriguing, as many genes within the G47 study have produced cold tolerance when overexpressed.

Potential applications. G3644 yields similar morphological phenotypes to those conditioned by G47, but the stress tolerance phenotypes are different. The utility of G3644 is not clear, but it appears to regulate at least some pathways in common with G47.

G3649 (SEQ. ID NO: 183 and 184; Oryza sativa)—Constitutive 35S

Background. G3649 is a rice ortholog of G47. The aim of this project was to determine whether G3649 produces comparable effects to G47 on morphology and stress tolerance when overexpressed in Arabidopsis.

Morphological Observations. 35S::G3649 lines exhibited similar phenotypes to those seen in G47 overexpression lines. The majority of lines showed dwarfing, particularly at early stages, were light green in coloration, slow developing, and had vertically oriented leaves. At later stages, many of the lines were late flowering and produced thick fleshy stems and leaves and had rather short inflorescence internodes. Several lines showed an abnormal branching pattern and short inflorescence internodes.

Physiology (Plate assays) Results. Three of 14 35S::G3649 lines showed increased cold germination in plate assays relative to wild-type controls.

Physiology (Soil Drought-Clay Pot) Summary. In soil-based drought assays, at least one line was more tolerant to drought and recovered from drought better than wild-type controls. A second line also recovered better from the drought treatment than controls.

Discussion. Direct fusion 35S::G3649 lines had morphological phenotypes similar to 35S::G47 lines: early dwarfing was observed in the majority of lines; at late stages, many lines were late flowering and exhibited thick leaves and stems. Based on these phenotypes, this protein appears to have comparable activity to G47.

Potential applications. G3649 produces similar morphological phenotypes to those conditioned by G47. G3649 appears to regulate at least some pathways in common with G47, and may be used to confer cold and drought tolerance to plants.

The G482 Clade and related Sequences
G481 (SEQ ID NO: 1 and 2; Arabidopsis thaliana)—Constitutive 35S

Background. G481 was included in the drought program based on the enhanced tolerance of 35S::G481 lines to drought related stress. This gene has been referenced in the public literature as AtHAP3a (Edwards et al., 1998) and NF-YB1 (Gusmaroli et al., 2001; 2002). Other than the expression data in these papers suggesting that the gene is ubiquitously expressed (with high levels in flower and/or silique) no functional data have been published. The aim of this study was to re-assess a larger number of 35S::G481 lines and compare its overexpression effects to those of other genes from the NF-Y family.

Morphological Observations. We have generated 35S lines for G481 using both the two-component system and a direct promoter fusion approach. Alterations in flowering time and a dark coloration were noted, but these effects were rather variable between different lines and plantings, suggesting that the phenotypes might be critically dependent on the specific level of G481 overexpression and/or were influenced by subtle changes in growth conditions (such as light intensity, temperature, air-flow etc.). In many instances, the 35S::G481 lines flowered at the same times as controls. However, when changes in flowering time were seen, in most cases, the clearest effect was a delay in the onset of flowering. In some lines, though, accelerated flowering was apparent.

Physiology (Plate assays) Results. Both two component and direct fusion 35S::G481 lines have been tested in plate based assays.

Initially, a set of ten two component lines were examined. Seedlings from four of these ten lines were less sensitive to ABA in a germination assay. Two of these lines also were more tolerant than wild-type in a cold germination assay.

Subsequently, a new batch of fifteen 35S::G481 direct promoter-fusion lines were tested. Five of the fifteen lines were more tolerant than controls in a cold germination assay. The same five lines showed enhanced vigor relative to wild-type seedlings on control plates in the absence of a stress treatment. Three of these five lines also showed more tolerance to sucrose in a sucrose germination assay (confirming the result obtained in our earlier genomics program). Three of these five lines were more tolerant than controls in a cold growth assays. Some of the five lines that performed well in the cold germination experiment also were more tolerant than controls in severe dehydration, mannitol and ABA germination assays.

Discussion. Both 35S::G481 two-component and direct fusion lines have now been extensively examined, and comparable phenotypes were obtained via each of these approaches. Changes in flowering time and a dark coloration were noted, but these effects were rather variable between different lines and plantings, suggesting that the phenotypes might be critically dependent on the specific level of G481 overexpression and/or were influenced by subtle changes in growth conditions (such as light intensity, temperature, air-flow etc.). In many instances, the 35S::G481 lines flowered at the same times as controls. However, when changes in flowering time were seen, in most cases, the clearest effect was a delay in the onset of flowering. In some lines, though, accelerated flowering was apparent. Thus, the switch to flowering appears to be finely balanced and there might be a specific range of G481 activity that determines whether a delay or acceleration of that switch occurs.

It should be emphasized that we have observed flowering time and stress tolerance-related phenotypes for many of genes from the NF-Y family; it is emerging that broad groups of the genes from across the entire family can influence these traits. Importantly the direction of the flowering time phenotypes do not appear to correlate with stress tolerance, since we have obtained convincing stress tolerance phenotypes with lines that showed either late or early flowering.

Potential applications. The results of these overexpression studies confirm our earlier conclusion that G481 and the related genes are excellent candidates for improvement of drought related stress tolerance in commercial species. Additionally, G481 related genes could be used to manipulate flowering time. The dark coloration and sucrose germination results obtained with 35S::G481 lines suggest that the gene might influence the regulation of photosynthesis and carbohydrate metabolism. As such, the gene may be used to enhance yield under a range of conditions, and not merely during water limitation.

G481 (SEQ ID NO: 1 and 2; Arabidopsis thaliana)—Vascular SUC2

Background. The aim of this project was to determine whether expression of G481 from a SUC2 promoter, which predominantly drives expression in a vascular specific pattern, is sufficient to confer stress tolerance that is similar to, or better than, that seen in 35S::G481 lines.

Morphological Observations. Overexpression of G481 from the SUC2 promoter produced a marked delay in the onset of flowering, dark green coloration, and increased rosette size at later stages of development.

Two-component lines containing an opLexA::G481 construct were supertransformed into a SUC2::LexA-GAL4TA promoter driver line. The majority of lines showed delayed flowering and dark coloration in both the T1 generation and each of three T2 lines that were examined.

As an alternative to the 2-component approach, we built a construct (P21522) that contains a direct promoter-fusion for SUC2::G481. The majority of these plants were late flowering and were dark in coloration. These effects were also observed in each of three T2 populations that were examined.

Physiology (Plate assays) Results. SUC2::G481 lines were more tolerant to cold conditions and a severe dehydration stress in plate based assays. The results were consistent for both direct fusion and two component lines; both sets of lines were more tolerant than controls in these two assays.

Physiology (Soil Drought-Clay Pot) Summary. Seven independent lines containing a SUC2::G481 direct fusion construct were tested in soil drought assays. One of these lines (#1691) was more tolerant than wild-type controls in two out of four runs of the experiment.

TABLE 35
SUC2::G481 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 1691 | DPF | 1.8 | 0.94 | 0.15 | 0.33 | 0.18 | 0.0051* |
| 1691 | DPF | 0.33 | 0.78 | 0.36 | 0.071 | 0.12 | 0.26 |
| 1691 | DPF | 1.2 | 1.0 | 0.49 | 0.079 | 0.13 | 0.18 |
| 1691 | DPF | 2.8 | 0.40 | 0.00020* | 0.78 | 0.064 | 0.000000000000000000000050* |

DPF = direct promoter fusion
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion: SUC2::G481 lines were obtained using both a direct-promoter fusion and a two-component approach. In either case, most of the lines were rather dark green in coloration, and showed delayed flowering. It is noteworthy that these effects were potentially stronger than those seen in 35S::G481 lines, indicating that high levels of G481 protein in the vascular system heavily influenced those phenotypes.

Potential applications. From our earlier studies, it was concluded that G481 could be applied to improve abiotic stress tolerance. These experiments indicate that SUC2 (or another vascular specific promoter) can be useful for optimizing G481 activity. Nonetheless, since the delayed flowering phenotype was potentially more severe than that observed with the 35S promoter, in certain target species such as soybean, the SUC2::G481 combination might actually exacerbate any off-types associated with delayed flowering and maturation.

Aside from abiotic stress tolerance traits, the SUC2::G481 combination may be of use in modifying flowering time. The dark coloration of SUC2::G481 lines might also be indicative of higher levels of chlorophyll or other pigments which could enhance photosynthetic capacity and yield.

G481 (SEQ ID NO: 1 and 2; Arabidopsis thaliana)—RNAi (GS)

Background. The aim of this project was to determine if G481 plays a critical role in stress tolerance by using an RNAi approach that was designed to specifically target G481 but not its related clade members.

Morphological Observations. Overall, lines harboring a G481 RNAi(GS) construct exhibited no consistent differences in morphology to wild-type controls.

A minority of T1 plants were noted to exhibit slight alterations in flowering time. A few lines were somewhat late flowering whereas a few others were marginally early flowering. Six populations were examined in the T2 generation: four lines appeared wild type.

Physiology (Plate assays) Results. Lines harboring a G481 RNAi (GS) construct were more tolerant to cold conditions in germination and growth assays.

Physiology (Soil Drought—Clay Pot) Summary. Three independent lines were tested in soil drought assays. The results indicate that the G481-RNAi (GS) construct might confer some level of drought tolerance.

One of the lines (#1672) showed significantly better survival that controls in two independent plantings. The other two lines each showed better survival on one plant date but not on a second plant date.

TABLE 36
G481-RNAi (GS) drought assay results:

| | | Mean | | p-value for | Mean | Mean | p-value for |
|---|---|---|---|---|---|---|---|
| | Project | drought | Mean drought | drought score | survival for | survival for | difference in |
| Line | Type | score line | score control | difference | line | control | survival |
| 1668 | RNAi (GS) | 1.8 | 0.74 | 0.080* | 0.36 | 0.19 | 0.0013* |
| 1668 | RNAi (GS) | 0 | 0.11 | 0.50 | 0 | 0.016 | 0.99 |
| 1669 | RNAi (GS) | 1.5 | 0.74 | 0.17 | 0.19 | 0.19 | 0.96 |
| 1669 | RNAi (GS) | 0.67 | 0.11 | 0.27 | 0.083 | 0.016 | 0.034* |
| 1672 | RNAi (GS) | 2.0 | 0.74 | 0.32 | 0.38 | 0.19 | 0.00028* |
| 1672 | RNAi (GS) | 0.67 | 0.11 | 0.31 | 0.095 | 0.016 | 0.020* |

Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Forty lines containing the G481 RNAi(GS) construct were studied, and overall, these lines appeared morphologically wild type. Nonetheless, minor changes in flowering time were noted in a minority of the lines, with some appearing slightly early flowering and others being slightly late flowering. These effects were subtle.

Surprisingly, G481 RNAi(GS) lines showed tolerance to cold in both germination and seedling growth assays. Additionally, evidence of drought tolerance was also obtained for G481 RNAi(GS) in soil based assays. It is interesting to compare these results with those obtained for a KO.G481 T-DNA allele: KO.G481 plants did not show an enhanced performance in plate assays, and in fact showed increased sensitivity to NaCl. No consistent difference to controls was seen for KO.G481 lines in soil drought assays, but the KO lines did show accelerated flowering.

The differences in phenotypes obtained with the G481 RNAi(GS) lines versus the KO.G481 alleles demonstrates that the RNAi(GS) construct either did not produce a complete knock-down of G481 activity, or influenced other components of the NF-Y family in a manner that had not been predicted. Thus, to determine the basis of the stress tolerance seen in these lines, substantial follow-up studies would be needed to assess the effects on other genes within the family. Nonetheless, the results confirm that the effects on stress tolerance seen with the NF-Y family are complex and are likely the result of genetic interactions between different members of the family. We have now performed a preliminary analysis on these RNAi(GS) lines to examine the effects on expression of a selected set of CCAAT family genes; we have found that there appeared to be a down-regulation of the HAP2 gene, G926 (which produces stress tolerance when knocked-out). Thus, the stress tolerance seen in the G481 RNAi(GS) lines may be an indirect result of down-regulation of G926.

Potential applications. These results indicate that enhanced stress tolerance can be obtained via knock-down approaches on the NF-Y family as well as by overexpression of genes encoding particular subunits. G481 RNAi (GS) lines may represent an indirect approach to reducing expression of G926, with reduced expression of G926 conditioning enhanced stress tolerance.

G481 (SEQ ID NO: 1 and 2; Arabidopsis thaliana)—Deletion Variant

Background. The aim of this project was to further refine our understanding of G481 function by use of a “dominant negative” approach in which truncated versions of the protein were overexpressed. Two different constructs (P21273 and P21274) were used to overexpress different portions of the G481 B domain (see sequence section for details).

Morphological Observations. Lines have been obtained for each of two different G481 deletion variant constructs (P21273 and P21274), each of which overexpresses a fragment of the G481 protein.

P21274 lines: some of these lines showed alterations in leaf shape, coloration, and, generally, delayed flowering time. However, such effects were of moderately low penetrance and were variable between lines and plant dates, suggesting that they could have been influenced by subtle changes in growth conditions. Many of the lines appeared wild type.

P21273 lines: Plants harboring this construct exhibited wild-type morphology at all stages of development.

Physiology (Soil Drought-Clay Pot) Summary. Overexpression lines for P21274, containing a truncated variant of G481 (see sequence section for details), exhibited enhanced drought tolerance in soil based assays.

Four independent lines were examined. Lines 1270 and 1267 yielded consistent results; both showed more tolerance than controls in each of the two whole pot experiments in they were tested. These lines, however, showed a wild-type performance in a single run of a split pot assay. Another line, 1266, showed significantly better survival in a split pot experiment and one run of a whole pot assay. However, in a different run of a whole pot assay, that line showed a worse performance than controls. The fourth line, 1269, showed a comparable performance to controls in both a whole pot and a split pot experiment.

TABLE 37
G481 deletion variant drought assay results:

| | | Mean | Mean | p-value for | Mean | Mean | |
|---|---|---|---|---|---|---|---|
| | Project | drought | drought | drought score | survival | survival for | p-value for difference |
| Line | Type | score line | score | difference | for line | control | in survival |
| 1267 | DV | 3.7 | 1.6 | 0.081* | 0.43 | 0.23 | 0.0010* |
| 1267 | DV | 1.5 | 0.70 | 0.10* | 0.18 | 0.12 | 0.18 |
| 1267 | DV | 2.3 | 1.8 | 0.35 | 0.32 | 0.25 | 0.41 |
| 1270 | DV | 5.3 | 1.6 | 0.0045* | 0.67 | 0.23 | 0.000000000034* |
| 1270 | DV | 4.0 | 0.70 | 0.000065* | 0.49 | 0.12 | 0.00000000000046* |
| 1270 | DV | 1.2 | 1.1 | 0.82 | 0.17 | 0.15 | 0.82 |

DV = Deletion variant; transcription factor dominant negative deletion, secondary domain (this truncated versions of G481 was overexpressed to drought tolerance conferred by particular parts of the protein. Such an approach can result in dominant negative alleles.)
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Transgenic Arabidopsis lines were created for each of the deletion variant constructs. Plants containing P21273 (which overexpressed an N-terminal portion of the B domain: amino acids 21-84) were morphologically similar to wild-type at all stages of development. When tested in plate based assays, these lines showed a wild-type response and were not tested in soil drought experiments.

Interesting phenotypes, however, were obtained for lines harboring the second construct (P21274) which overexpressed an N-terminal portion of the B domain: amino acids 51-117. This fragment of the G481 protein was predicted to be incapable of DNA binding, but could potentially have associated with other HAP subunits. The P21274 lines showed tolerance to NaCl in germination assays and exhibited drought tolerance in soil based assays. Developmental changes were also noted in some of the lines. Among the primary transformants, a delay in the onset of flowering, along with long, narrow, slightly dark leaves was observed in about one third of the lines. A number of T2 populations were morphologically examined; changes in leaf shape were apparent, and some lines showed slightly delayed flowering. However, in other T2 lines, a slight acceleration in flowering was noted. Thus, the effects on flowering time were somewhat unstable, and could have depended on the specific level of overexpression, along with environmental factors such as light intensity and temperature.

In soil drought physiology experiments performed on P21274 lines, apparently higher levels of chlorophyll and carotenoids compared to wild-type were observed at a moderately droughted state. One of the lines (#1270, which showed the strongest drought tolerance phenotype) also showed a higher level of proline versus wild-type under a well-watered condition and under a mild drought. A slightly elevated ABA level was also apparent in this line under mild drought.

It should be noted that late flowering and stress tolerance have been observed in plants overexpressing the full-length version of G481 and in a KO.G485 line. Thus, the phenotypes seen in the deletion variant lines could have been due to interference of the truncated form of G481 with other components of the CCAAT-binding complex. The results also raise the possibility that stress tolerance produced by overexpression of the native full-length version of G481 might be derived from a similar “dominant negative” type effect.

Potential applications. Based on the results of our overexpression studies, G481 and its related paralogs are excellent candidates for improvement of drought related stress tolerance in commercial species. This deletion variant study adds further insight into how G481 might influence stress tolerance. It is possible that the truncated form of G481 confers more robust stress tolerance than the native full-length form. The results from this study indicate that both stress tolerance and flowering time traits can be obtained from overexpression of a fragment of a CAAT binding factor, and are not dependent on the full-length protein being overexpressed.

G481 (SEQ ID NO: 1 and 2; Arabidopsis thaliana)—Super Activation (C-GAL4-TA)

Background. The aim of this project was to determine whether the efficacy of the G481 protein could be improved by addition of an artificial GAL4 activation domain at the C-terminus.

Morphological Observations. Overexpression of a G481-GAL4 fusion, produced a striking acceleration in the onset of flowering (1-2 weeks under 24-hour light conditions) and a severe reduction in overall plant size.

T1 lines:

The above effect was highly penetrant and was observed in the majority of plants from three separate batches of T1 lines.

T2 lines:

Accelerated flowering was also observed, to varying extents in the T2 generation populations.

Physiology (Plate assays) Results. Fifteen different 35S::G481-GAL4 lines were tested in plate based assays, spanning two different plant dates. Positive results were obtained in a number of different assays as shown in the table below. For unknown reasons, though, lines from the 521-531 set showed a lower frequency of phenotypes than those from the 1621-1640 set.

Substantially enhanced tolerance, relative to controls, was seen NaCl germination (5/15 lines) and heat germination (5/15 lines).

A number of the lines which showed a strong performance in these assays also performed better than wild-type in other assays such as severe dehydration and chilling growth. Additionally, a number of lines were generally larger and more vigorous than wild-type controls on regular control growth and germination MS plates.

Discussion. Several independent transgenic lines were generated for this study. The majority of these plants displayed a striking acceleration in the onset of flowering, as well as a severe reduction in overall plant size. Interestingly, the flowering results seen here were largely opposite to the late flowering seen from overexpression of the wild-type form of the G481 protein (see 35S::G481 report). It should be emphasized that the effects on flowering time obtained with 35S::G481-GAL4 were most comparable to those seen in 35S::G482 or 35S::G485 lines. Thus, the new domain added at the C-terminus had switched the effects on flowering produced overexpression of the native G481 protein.

Lines tested in soil drought assays gave rather inconclusive results. Two independent lines showed significantly better survival than controls in one of the runs of the assay. Nonetheless, other lines actually performed worse than wild type in later runs of the assay. Interestingly, though, the lines which showed this poor performance were the ones that exhibited the strongest effects on flowering time; thus, there might exist a threshold level of G481-GAL4 activity, above which the effects become negative.

Potential applications. Based on the results of our overexpression studies, the G481-GAL4 combination could be used to modify flowering time. Nonetheless, it might be necessary to select plants with an optimum level of G481-GAL4 expression in order to achieve both early flowering and drought tolerance. In the light of the delayed maturation off-type seen in 35S::G481 soy lines, the G481-GAL4 combination may be used in that species to achieve drought tolerance without a delay in maturity.

G481 (SEQ ID NO: 1 and 2; Arabidopsis thaliana)—RNAi (Clade)

Background. The aim of this project was to determine if the G482 clade plays a critical role in stress tolerance by using an RNAi approach that was designed to specifically target the G482 clade members (G481, G482, G485, G1364 and G2345).

Morphological Observations. G481-RNAi (clade) lines exhibited complex changes in flowering time and leaf shape/coloration.

Three independent batches containing a total of 43 T1 lines harboring G481 RNAi (clade) constructs were examined. Plants from each of these three sets of lines exhibited a clear delay in the onset of flowering (up to 2-3 weeks later than wild-type under 24 hour light conditions) and displayed leaves that were long, narrow and slightly dark in coloration. Several lines were tiny and darker green at early stages. A number of lines showed very long petioles.

Physiology (Plate assays) Results. G481-RNAi (clade) lines were more tolerant than control plants to salt (5 of 24 lines tested), mannitol (3 of 24 lines), sucrose (3 of 24 lines), growth in heat (9 of 24 lines), severe desiccation (3 of 24 lines), and growth in cold (5 of 24 lines). One line (#1322) was much more tolerant to heat than wild-type controls during germination in duplicate plate assays conducted.

Discussion. Two different sets of RNAi molecules were designed to interfere with the expression of the G482 clade. One variant (P21305) was based on sequences from G485 and G2345, while the other variant (constructs P21159 and P21300, which were identical to each other) was modeled on G482 and G2345. Both variants contained base-pair mutations intended to optimize homology to the clade.

Each of the RNAi (clade) constructs produced similar, but complex effects on plant development. In the T1 generation, many of the lines for each construct exhibited a clear delay in the onset of flowering (up to 2-3 weeks later than wild-type under 24 hour light conditions) and displayed leaves that were long, narrow and slightly dark in coloration. However, a number of lines were later examined in the T2 generation. In some cases, the plants showed a comparable phenotype to that seen in the T1 and were late flowering. However, unexpectedly, some of the T2 populations were early flowering, even though the parental plant had been late flowering. Additionally, for a given T2 line, in some instances a flowering time phenotype was apparent in one planting, but was not seen on a different plant date. Thus, the effects of the transgene appeared to change between generations, and could have depended on subtle variables such as temperature and light intensity, which might have differed between plantings.

When tested in plate-based physiology assays, lines for each of the constructs showed evidence of stress tolerance. In particular, lines of both constructs displayed enhanced tolerance in a heat growth assay. Lines for one of the constructs (P21305) also showed consistently better tolerance than controls to a mannitol germination assay. Many of the lines also were larger and more vigorous than wild-type seedlings when grown on control plates in the absence of a stress treatment.

Potential applications. These results indicate that enhanced stress tolerance can be obtained via knock-down approaches on the NF-Y family as well as by overexpression of genes encoding particular subunits.

The morphological effects seen in these RNAi lines indicate that a knock-down approach could also be applied to the NF-Y family to modify flowering time. Also the dark coloration seen in the lines could indicate an increase in chlorophyll levels; thus the gene may be used to improve photosynthetic capacity, yield, and nutritional quality.

G481 (SEQ ID NO: 1 and 2; Arabidopsis thaliana)—Knockout (KO)

Background. The aim of this project was to determine if G481 plays a critical role in stress tolerance by knocking out its expression using T-DNA insertional mutagenesis. A null mutant for G481 would also assist with genetic analysis (for example, via its combination with other KO and overexpressing lines) to allow a more refined understanding of where the gene is positioned in stress tolerance pathways relative to other genes.

Insertion line SALK_{—}032272 (NCBI acc. no. BH612182, version BH612182.1; GI:1805975; SALK_{—}032272 Arabidopsis thaliana TDNA insertion lines Arabidopsis thaliana genomic clone SALK_{—}032272, genomic survey sequence): BLAST analysis of the sequence from the insertion point deposited in GenBank by SALK indicates that the T-DNA in this line is integrated approximately 1300 bp downstream of the G481 start codon.

Insertion line SALK-109993 (NCBI acc. no. BZ664699; version BZ664699.1; GI:28181591; SALK-109993.42.55.x Arabidopsis thaliana TDNA insertion lines Arabidopsis thaliana genomic clone SALK_{—}109993.42.55.x, genomic survey sequence): BLAST analysis of the sequence from the insertion point deposited in GenBank by SALK indicates that the T-DNA in this line is integrated approximately 115 bp downstream of the G481 start codon.

RT-PCR and protein blot experiments performed on tissue from the homozygous plants did not detect G481 transcript or protein compared to wild-type controls. Thus, both of the T-DNA insertion alleles appear to be null mutations.

Morphological Observations. We have isolated homozygous KO.G481 populations for two independent T-DNA insertion alleles derived from the SALK collection. In each case, the plants showed accelerated flowering, but this phenotype varied in penetrance across different plant dates, and was likely influenced by subtle differences in growth conditions.

Physiology (Plate assays) Results. Homozygotes for a T-DNA insertion within G481 (SALK-032272) were more sensitive to sodium chloride in a germination assay.

Discussion. We have isolated homozygous KO.G481 populations for two independent T-DNA insertion alleles derived from the SALK collection. Both of these appear to be null mutations, based on the absence of G481 transcript or protein. In each case, the plants showed accelerated flowering, but this phenotype varied in penetrance across different plant dates, and was likely influenced by subtle differences in growth conditions.

One of the alleles was tested in plate-based physiological assays, the plants were more sensitive to sodium chloride germination, having lower germination efficiency than wild-type plants. This result was reproduced in a number of repeats of the experiment. This finding supports the notion that G481 has an endogenous role in abiotic stress protection. As yet, though, we have not found a clear-cut difference between the KO lines and wild-type in soil based drought assays.

Potential applications. The results of this study complement the findings of our overexpression experiments and indicate that G481 has an endogenous role in affording stress tolerance in Arabidopsis. This supports our earlier conclusions that the gene may be applied to engineer improved drought and abiotic stress tolerance in commercial crops. The accelerated flowering seen in the KO lines indicates that G481 might act as a floral repressor as part of its native role, and that the gene may be applied to modify flowering time traits.

G485 (SEQ ID NO: 17 and 18; Arabidopsis thaliana)—Constitutive 35S

Background. G485 is a non-LEC1-like member of the HAP3 (NF-YB) sub-group of the Arabidopsis CCAAT-box binding transcription factor family. Along with G482, this gene occupies a separate sub-clade within the phylogeny to G481. G485 has been referenced as sequence 1042 from patent application WO0216655 on stress-regulated genes, transgenic plants and methods of use. G485 was reported therein to be cold responsive in a microarray analysis (Harper et al., 2002). The gene has also been designated as NF-YB3 by Gusmaroli et al. (2001; 2002).

During the earlier genomics program, we examined knockout and overexpression lines for G485. While no effects were noted at that time for drought stress related phenotypes, effects on flowering time were observed for plants overexpressing G485. These plants had accelerated flowering, bolting up to one week earlier than wild-type plants grown under 24 hr lights. These studies, combined with studies on plants lacking G485 expression (see the KO.G485 report) demonstrate that G485 is sufficient to act as a floral activator, and is also necessary in that role within the plant.

The aim of this study was to re-assess the effects of overexpression of G485 using a two-component system and to determine if this gene can confer enhanced stress tolerance in a manner comparable to G481.

Morphological Observations. Many of the 35S::G485 two-component lines exhibited a marked acceleration in the onset of flowering and generally formed flower buds 1-2 weeks sooner than wild type under continuous light conditions. Many of the lines also showed a reduction in rosette biomass compared to wild type. In fact, three of twenty lines showed a severe dwarf phenotype and did not survive to maturity. Early flowering was exhibited by 11/20 of the T1 lines (#301, 302, 303, 304, 306, 307, 309, 313, 315, 317, and 319). The remaining lines appeared wild type, apart from lines 310 and 314 which were noted to be slightly delayed in the onset of flowering. Line 14 was also infertile and failed to yield seed.

Flowering time was also assessed in a number of T2 populations: plants from the T2-302, T2-305, T2-307, T2-309, and T2-319 all displayed early flowering comparable to that seen in the parental lines. Plants from the T2-310 and T2-311 populations flowered at the same time as controls.

(All of the ten 2-component lines submitted for physiological assays showed segregation on selection plates in the T2 generation that was compatible with the transgene being present at a single locus.)

A new set of 35S::G485 direct promoter-fusion lines (361-380) was subsequently obtained; 19/20 of the T1 plants were noted to be early flowering, slightly small and slightly pale in coloration. One of the lines (T2-369) was grown in the T2 generation and showed early flowering. A pair of lines obtained during the initial genomics program (lines 76 and 77) were also examined; line 77 flowered early, whereas line 76 appeared wild type.

Physiology (Plate assays) Results. We had previously observed that G485 overexpressing lines behaved similarly to the wild-type controls in all physiological assays performed. However, when seedlings from ten new two-component lines overexpressing G485 were examined, tolerance to several stress related conditions were observed. Eight of ten lines were more tolerant to salt stress in a germination assay compared to wild-type seedlings. Several salt tolerant lines were also less sensitive to sucrose, ABA, and cold stress in separate germination assays.

In subsequent experiments, a new set of ten 35S::G485 direct fusion lines were tested. These lines were more tolerant than controls in a cold growth assay, but appeared wild type in the other assays. The differences in results seen between one and two-component lines could be attributed to different ranges of G485 overexpression levels being attained via these approaches.

Physiology (Soil Drought-Clay Pot) Summary. 35S::G485 (2-component) overexpression lines showed evidence of drought tolerance when tested in soil based assays. Three independent lines were tested in a whole pot assay; two of these lines (lines 310 and 319) showed better tolerance and/or recovery than wild-type on at least one of three different plant dates. Line 310 also performed better than wild-type in a single run of a split pot soil drought assay (line and controls together in same pot). A third (2-component) line (#311) performed better than wild-type in two runs of the assay, but for unknown reasons, performed worse than wild-type when the assay was repeated for a third time. Such variation in results between different plantings suggests that the drought resistance phenotype could have been influenced by factors such as growth temperature, air-flow, and light intensity, which might have varied between different runs of the experiment.

Four independent 35S::G485 direct promoter-fusion lines were also put through soil assays, but these showed no consistent improvement in drought tolerance relative to wild type. In fact, two of the direct promoter-fusion lines performed worse than wild-type on one of the dates tested.

The differences in performance observed between 2-component and direct fusion lines for G485 suggest that there might be a particular range of G485 levels which are effective under drought conditions.

TABLE 38
35S::G485 drought assay results:

| | | | | p-value for | | | p-value for |
|---|---|---|---|---|---|---|---|
| | Project | Mean drought | Mean drought | drought score | Mean survival | Mean survival | difference in |
| Line | Type | score line | score control | difference | for line | for control | survival |
| 310 | TCST | 0.83 | 0.50 | 0.43 | 0.13 | 0.095 | 0.47 |
| 310 | TCST | 1.5 | 0.90 | 0.24 | 0.23 | 0.14 | 0.067* |
| 310 | TCST | 0.30 | 0.10 | 0.30 | 0.014 | 0.043 | 0.17 |
| 310 | TCST | 3.6 | 2.3 | 0.045* | 0.51 | 0.33 | 0.045* |
| 319 | TCST | 1.4 | 0.10 | 0.00056* | 0.21 | 0.036 | 0.000096* |
| 319 | TCST | 1.3 | 0.50 | 0.024* | 0.14 | 0.11 | 0.47 |

TCST = two-components-supertransformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. As was seen for the direct 35S promoter fusion lines generated for our earlier genomics program, plants expressing 35S::G485 via the two-component system consistently flowered 1-2 weeks earlier than wild-type plants under 24 hr light. (Similar effects on flowering time were noted with other G481-related genes such as G482 and G1820.)

Evidence of drought tolerance was seen in the 2-component lines but was not apparent in the direct fusion lines. There was no clear-cut association between drought tolerance and flowering time, since drought tolerance was obtained in a line that was early flowering and in a line that was wild type.

35S::G485 lines (both the direct fusion lines and the two-component lines) were also examined in “single pot” soil drought experiments and a number of physiological parameters were measured. No consistent differences to controls were seen.

The difference in results between the two-component and direct fusion approached might be accounted for by the two-component system giving an amplification of G485 overexpression relative to that found in 35S::G485 direct fusion lines.

Potential applications. The results of this study bolster our conclusion that G481 and the related genes such as G485 are excellent candidates for improvement of abiotic stress tolerance (such as drought, cold, and salt) in commercial species.

Additionally, G485 could be used to manipulate flowering time, and may be particularly useful in situations where an acceleration or induction of flowering was desired.

G485 (SEQ ID NO: 17 and 18; Arabidopsis thaliana)—KO

Background. We d previously examined KO and overexpression lines for G485. While no effects were noted at that time for drought stress related phenotypes, effects on flowering time were observed for plants overexpressing G485. These plants had accelerated flowering, bolting up to 1 week earlier than wild-type plants grown under 24 hr lights. The knock out line appeared wild type, but we subsequently reported preliminary data for a SALK line (SALK_{—}062245

(NCBI acc. no. BH791968, version BH791968.1; GI: 19887127; SALK_{—}062245.42.85.x Arabidopsis thaliana TDNA insertion lines Arabidopsis thaliana genomic clone SALK_{—}062245.42.85.x, genomic survey sequence). that showed delayed flowering. These studies indicated that G485 is both sufficient to act as a floral activator, and is also necessary in that role within the plant. The aim of this study was to use a KO approach to determine whether G485 has a native role in stress response pathways.

A G485 T-DNA insertion line derived the SALK collection (SALK_{—}062245) was obtained from the ABRC at Ohio State University. BLAST analysis of the sequence from the insertion point deposited in GenBank by SALK indicates that the T-DNA in this line is integrated near the end of the gene, approximately 458 bp downstream of the G485 start codon.

Morphological Observations. We identified two homozygous plants (lines 341, 346), by PCR genotyping, among seven individual plants germinated from the seed lot supplied by the ABRC. Both of these homozygotes were later flowering compared to controls (24-hour light conditions).

Selfed seed was collected from lines 341 and 346 and a batch of 18 progeny from each was grown, verified as being homozygous, and morphologically examined under 24-hour light conditions. Both these batches of plants again showed a moderate delay in flowering (approximately 1-2 weeks after controls). Seeds were collected from the 18 plants from homozygous line 341 and the next generation were examined on multiple independent plant dates. In each of these plantings, a delay in the onset of flowering was observed, and in some cases the plants took on a dark coloration.

RT-PCR experiments performed on tissue from the homozygous plants initially did not detect G485 transcript compared to wild-type controls, suggesting that the T-DNA insertion had resulted in a null mutation. However, in later experiments we detected a (weak) larger band, using primers spanning the putative T-DNA insertion site. Our interpretation is that this allele comprises a T-DNA border inserted into the 3′ end of the G485 gene. Currently, it is not known whether the allele is functional.

Physiology (Plate assays) Results. Homozygotes for a T-DNA insertion within G485 (SALK_{—}062245) were more tolerant than wild-type seedlings in germination assays containing sodium chloride and ABA. Such results were obtained with seed lots derived from multiple different homozygous plants carrying the SALK_{—}062245 insertion.

Physiology (Soil Drought-Clay Pot) Summary. Homozygotes for a T-DNA insertion within G485 (SALK-062245) showed enhanced drought tolerance in soil based assays.

### G485 Knockout

G485 plants were tested on three independent plant dates; more tolerance to and better recovery from drought than controls was obtained on two of those three dates. On the third date, the plants showed a wild-type performance.

TABLE 39
G485 Knockout drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 341-1 | KO | 1.7 | 0.73 | 0.019* | 0.23 | 0.13 | 0.00084* |
| 341_MIX | KO | 2.4 | 1.3 | 0.10* | 0.41 | 0.26 | 0.0081 |
| 341_MIX | KO | 1.6 | 1.4 | 0.59 | 0.43 | 0.43 | 1.0* |

KO = knockout
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Homozygotes for a G485 T-DNA insertion line (SALK_{—}062245, which carried the T-DNA at the 3′ end of the gene) were confirmed to be late flowering, and showed a 1-2 week delay in flowering under 24-hour light conditions. Additionally, following the onset of flowering, in some of the plantings, the plants were somewhat dark in coloration relative to wild type. In plate based assays, this KO line exhibited greater tolerance than controls in separate ABA and NaCl germination assays. The KO.G485 line also showed a better performance than controls in soil drought assays. At present, it is not clear whether the allele used in these experiments was null or retained some degree of activity. Our RT-PCR experiments indicate that the allele comprises a T-DNA border inserted into the end of the G485 gene. An expressed product, which is larger than the native transcript, is detectable.

Results for the KO.G485 line are very comparable to the data obtained for G481 overexpression lines. Thus, there could be antagonistic interactions between different genes from the NF-Y family. The physiological basis of the stress tolerance in this KO line is therefore not yet clear and might be related either to parameters that were not measured in the physiology experiments or to changes that were too subtle to detect.

Potential applications. The data from these KO studies confirm that G485 is part of the genetic networks that regulate flowering time, and as such, the gene may be used to manipulate the onset of flowering in commercial species. The data from our plate and soil drought assays, also demonstrate that G485 regulates stress tolerance; confirming that the gene is a good candidate for the modification of stress tolerance traits. In particular, the results indicate that it might be possible to modify abiotic stress tolerance and flowering time by knock-down approaches, such as by screening for (naturally) occurring alleles of G485 orthologs in target crop species. Technology such as TILLING (McCallum, C., et al., 2000) could be used as part of such approaches.

G482 (SEQ ID NO: 27 and 28; Arabidopsis thaliana)—Constitutive 35S

Background. G482 is a non-LEC1-like member of the HAP3 (NF-YB) sub-group of the Arabidopsis CCAAT-box binding transcription factor family. Along with G485, this gene occupies a separate sub-clade within the phylogeny to G481. G482 has been referenced in the public literature as AtHAP3b and AF-YB2 (Edwards et al., 1998; Gusmaroli et al., 2001; 2002). Data in these papers suggest that the gene is constitutively expressed.

We have previously observed that plants overexpressing G482 were NaCl tolerant in a germination assay. The aim of this study was to re-assess the effects of overexpression of G482 (using a two-component system) and to compare these to the effects of changes in G481 activity.

Morphological Observations. We have now generated 35S lines for G482 using the two-component system; two batches of T1 lines (321-341 and 341-360) were examined and many of the plants showed a striking acceleration of flowering (1-2 weeks sooner than wild-type) under 24 hour light conditions.

The early flowering effect was seen in many of the lines examined. The majority of 35S::G482 lines also displayed a slight reduction in overall size; in fact a number of lines were very small and did not survive to maturity. Comparable effects on flowering time were also seen in five of six T2 populations. Plants from a sixth T2 population (T2-346) were slightly small and slow growing, but otherwise appeared wild type.

All of the ten 2-component lines submitted for physiological assays showed segregation on selection plates in the T2 generation that was compatible with the transgene being present at a single locus.

We isolated a new batch of 35S::G482 direct promoter fusion lines. In contrast to the two component lines, early flowering was observed at relatively low frequency and was apparent in only 3/20 of the lines.

The basis for the difference in result between the direct fusion and two-component lines is unknown. However, it could relate to the possibility that higher levels of G482 activity were obtained with a two-component approach.

Physiology (Plate assays) Results. During our earlier genomics program, plants overexpressing G482 showed increased seedling growth relative to wild-type when germinated on high salt media. A similar tolerance to osmotic stress was observed in the present studies.

Initially, two-component lines were tested in these plate assays. Five out of ten lines had better seedling vigor versus controls when germinated on plates containing mannitol. Three of these lines also had more vigor in a heat germination assay compared to wild-type seedlings.

Subsequently, we tested a new set of direct promoter-fusion lines. These lines showed a lower frequency of phenotypes than the two component lines, and positive results were not seen in the mannitol and heat assays. Nonetheless, two of the lines did show an enhanced tolerance in the NaCl germination assay, confirming our initial result from the genomics program.

Physiology (Soil Drought-Clay Pot) Summary. Overexpression of G482 conferred enhanced drought tolerance under soil grown conditions.

Positive results were obtained in the “whole pot” assay. Three independent two component lines were each tested in four different plantings. Two of the lines showed a significantly enhanced performance across multiple plantings (#351 on two of four dates and #354 on three of four dates).

TABLE 40
35S::G482 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 351 | TCST | 1.0 | 0.37 | 0.12 | 0.11 | 0.063 | 0.17 |
| 351 | TCST | 1.0 | 1.0 | 1.0 | 0.13 | 0.13 | 0.93 |
| 351 | TCST | 1.9 | 0.70 | 0.074* | 0.39 | 0.18 | 0.00016* |
| 351 | TCST | 2.4 | 0.40 | 0.00021* | 0.44 | 0.036 | 0.00000000028* |
| 354 | TCST | 2.0 | 0.37 | 0.0015* | 0.19 | 0.063 | 0.00068* |
| 354 | TCST | 1.0 | 1.0 | 1.0 | 0.15 | 0.13 | 0.57 |
| 354 | TCST | 1.7 | 1.0 | 0.14 | 0.54 | 0.37 | 0.0042* |
| 354 | TCST | 1.9 | 0.90 | 0.041* | 0.31 | 0.11 | 0.000071* |

TCST = two-components-supertransformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. G482 (two-component) overexpression lines showed a striking 1-2 week acceleration in flowering time. Additionally, many of the lines showed a slight overall decrease in size, and some lines did not survive to maturity. Similar effects on flowering time were noted with other G481-related genes such as G485 and G1820.

The effects on flowering time were not originally noted in our earlier genomics screen when 35S::G482 direct fusion lines were examined. To re-assess that result, we isolated a new set of direct fusion lines; the majority of plants flowered at a similar time to controls, and early flowering was seen in only a small number (3/20) of the lines. The new set of direct fusion lines was also tested in plate based assays. Positive phenotypes were obtained at a lower frequency than with the two-component lines; two of ten 35S::G482 direct fusion lines showed salt tolerance in a germination assay, out in most of the assays, a wild-type response was observed. The direct fusion lines were not tested in soil based assays.

It is possible that the higher penetrance of flowering time and stress resistance phenotypes obtained in the two-component lines versus direct fusion lines resulted from higher levels of G482 overexpression in the former.

Potential applications. The results of this study strengthen our conclusion that G481 and its related genes are excellent candidates for improvement of drought related stress tolerance in commercial species. Additionally, G482 could be useful for manipulating flowering time.

G482 (SEQ ID NO: 27 and 28; Arabidopsis thaliana)—Vascular SUC2

Background. G482 is a close Arabidopsis homolog of G481. The aim of this project was to determine whether expression of G482 from a SUC2 promoter, which drives expression in a vascular specific pattern, would produce comparable effects on morphology and stress tolerance to that seen with a 35S promoter.

Morphological Observations. Lines in which G482 was expressed from the SUC2 promoter have now been obtained using the 2-component system. These lines showed a very marked acceleration in the onset of flowering under 24-hrs light, were slightly pale in coloration, and were generally more developmentally advanced than controls at all developmental stages.

A few lines were slightly small at early stages. At later stages most lines were early developing and had lighter green rosettes than controls.

Physiology Results. In plate experiments, except for occasional SUC2::G482 lines which showed positive results in cold and dehydration assays, no consistent difference was observed compared to controls.

Discussion. SUC2::G482 lines have been generated using a two component system. These lines were very similar to 35S lines and showed a very clear-cut acceleration of flowering. However, the phenotype was obtained at an even higher penetrance in the SUC2::G482 lines than in the 35S::G482 lines. Thus, high levels of G482 protein in the vascular system appear to be sufficient to effect this flowering phenotype (the result is also interesting in the light of our finding that SUC2::G481 lines showed similar phenotypes to 35S::G481 lines).

Potential applications. Given the effect on flowering time, the SUC2::G482 combination may be useful for modifying the onset of flowering, particularly in instances where an acceleration or induction of flowering is desired.

G489 (SEQ ID NO: 45 and 46; Arabidopsis thaliana)—Constitutive 35S

Background. G489 is a member of the HAP5 (NF-YC) subfamily of the CCAAT-box binding transcription factors. Since the NF-Y factors are known to act as trimeric complexes (comprising YA, YB, and YC subunits) we are testing whether genes for the other subunits (YCs and YAs) can confer drought tolerance in a comparable manner to genes encoding the YB subunit class to which G481 belongs.

We initially had observed that plants overexpressing G489 were tolerant of NaCl and mannitol in separate growth assays. Morphologically, the plants were similar to wild type. The aim of this study was to re-assess 35S::G489 lines for drought-related stress tolerance. Also, we sought to test whether use of a two-component overexpression system would produce any strengthening of the phenotype relative to the use of a 35S direct promoter-fusion.

Two new sets of 35S::G489 lines (301-320 and 421-440) have been obtained as part of the drought program.

Lines 301-320 harbored a 35S direct promoter-fusion construct (P51). The second batch of plants (421-440) overexpress G489 via the two component system.

Morphological Observations. Neither of the two sets of 35S::G489 plants showed any consistent differences in morphology to wild-type controls in either the T1 or T2 generations.

Physiology (Plate assays) Results. Lines harboring a 35S direct promoter-fusion construct or overexpressing G489 via the two component system have now been analyzed in abiotic stress assays. Five out of ten lines with a direct promoter fusion (P51) were more tolerant than wild-type seedlings in a cold germination assay. Three other lines were more tolerant in a chilling growth assay. Two lines were tolerant to dehydration stress in a severe drought plate based assay.

Only two lines were more tolerant to dehydration stress and one other line tolerant to cold in germination assays for lines harboring the two-component system for driving 35S expression.

This result is the opposite of that observed when we compared direct promoter fusions versus two-component systems. Normally the two-component system is more robust than direct promoter fusions in generating phenotypes.

Discussion. New sets of 35S::G489 lines derived from direct promoter fusions or the two-component system were created and characterized morphologically. Neither of the two sets of plants showed any consistent differences in morphology from wild type controls.

Potential applications. Based on the results of these and previous experiments, genes for other NF-Y subunits, as well as the YB class, can produce abiotic stress tolerance when overexpressed. G489, as a member of the YC class is evidence of this; the gene could potentially be applied to enhance tolerance to abiotic stress such as drought and cold.

G926 (SEQ ID NO: 51 and 52; Arabidopsis thaliana)—KO

Background. G926 is an Arabidopsis gene which is a member of the HAP2 (NF-YA) subfamily of the CCAAT-box binding transcription factors. Since the NF-Y factors are known to act as trimeric complexes (comprising YA, YB, and YC subunits) we are testing whether genes for the other subunits (YCs and YAs) can confer drought tolerance in a comparable manner to genes encoding the YB subunit class to which G481 belongs.

A G926 knockout line was isolated and carries a T-DNA insertion at ˜425 bp downstream of the G926 ATG. This line was examined morphologically and tested in stress assays, and was found to show tolerance to abiotic stresses such as NaCl, sucrose, and ABA.

Since the original genomics screen, RT-PCR has been performed to confirm the absence of a wild-type G926 transcript (using a pair of primers that spanned the T-DNA insertion) in this line. However, it should be noted that a product was obtained in RT-PCR experiments with a pair of primers that were both 5′ to the T-DNA insertion point. Thus, it is possible that a truncated variant of G926 was expressed in these lines, but it is not clear whether or not the allele would have been functional.

Morphological Observations. During the original genomics screens, we analyzed a homozygous KO.G926 line. Plants from that line showed wild-type morphology. Additional homozygous plants have been examined under 24-hour light conditions. These plants also exhibited no consistent difference in morphology to wild-type controls.

Physiology (Plate assays) Results. Lines with a knockout of G926 were more tolerant than wild-type seedlings in several abiotic stress assays, including, NaCl (8 of 10 lines tested), ABA (10 of 10 lines), sucrose germination assays (8 of 10 lines), severe dehydration (4 of 10 lines), and a chilling growth assay (10 of 10 lines). Ten different seed lots derived from individual homozygous plants for the same T-DNA insertion allele were tested in these assays.

Physiology (Soil Drought-Clay Pot) Summary. Two separate lines with a knockout of G926 showed better performance in a soil-based drought assay than controls, and one of these lines recovered better from the drought treatment than the controls.

Discussion. We have so far been unable to identify additional KO.G926 alleles in the public collections. However, we have now re-analyzed our original KO.G926 line and confirmed the stress tolerance effects seen in plate based assays.

Potential applications. G926 may be used to regulate abiotic stress tolerance traits. In particular, the strong positive data from plate based assays support the notion that a knock-down approach on NF-Y family genes may be a viable method of achieving stress tolerance in commercial crops such as maize and soy.

G928 (SEQ ID NO: 399 and 400; Arabidopsis thaliana)—Constitutive 35S

Background. G928 is an Arabidopsis member of the HAP2 (NF-YA) subfamily of the CCAAT-box binding transcription factors. Since the NF-Y factors are known to act as trimeric complexes (comprising YA, YB, and YC subunits) genes for the other subunits (YCs and YAs) such as G928 were tested to determine whether these sequences can confer drought tolerance in a comparable manner to genes encoding the YB subunit class to which G481 belongs.

Morphological Observations. 35S::G928 lines exhibited a wild-type morphology.

Physiology (Plate assays) Results. Ten of 10 lines tested were more tolerant than wild type in a cold germination assay. Five of those lines were also more tolerant to sucrose in a separate germination assay.

Discussion. Drought assays have not yet been performed with G928 constitutive overexpressing lines. However, the tolerance in sucrose and cold germination assays that was observed suggests that G928 overexpression will confer tolerance to abiotic stress, including hyperosmotic stresses.

Potential applications. The abiotic stress results coupled with the wild-type morphology and development exhibited by these lines, this sequence is an excellent candidate for conferring stress tolerance in commercially important plants.

G1836 (SEQ ID NO: 47 and 48; Arabidopsis thaliana)—Constitutive 35S

Background. G1836 is a member of the HAP5 (NF-YC) subfamily of the CCAAT-box binding transcription factors. Since the NF-Y factors are known to act as trimeric complexes (comprising YA, YB, and YC subunits) we are testing whether genes for the other subunits (YCs and YAs) can confer drought tolerance in a comparable manner to genes encoding the YB subunit class to which G481 belongs.

Plants overexpressing G1836 were somewhat paler green than the wild-type controls in morphology assays. 35S::G1836 lines also showed enhanced tolerance in salt stress germination assays. The aim of this study was to re-assess the effects of G1836 on tolerance to drought-related stress.

Morphological Observations. Three independent batches of 35S::G1836 (2-component) T1 lines (301-313; 361-372; 381-386) were examined. Many of the plants showed a variety of morphological changes including: reduced overall size, abnormal leaf shape and coloration (some lines were slightly yellow), vertically oriented leaves, slightly delayed flowering or slow growth, reduced apical dominance, and floral abnormalities that resulted in poor fertility. It should be noted, however, that a number of lines showed no consistent differences to wild type. The effects seen with 2-component are similar to those exhibited by the 35S::G1836 direct fusion lines.

Line 306 was small and showed the pleiotropic effects described above. Line 384 also showed the showed the pleiotropic effects described.

In the T2 generation, one of six T2-306 plants was slightly pale with serrated leaves, five of six appeared wild type.

T2-384 plants were grown on two different plant dates. On one of these dates, the plants appeared wild type, but on the second date were pale and slightly early flowering. This response might depend on variables such as growth temperature, which could have differed between the two plantings.

Physiology (Plate assays) Results. G1836 overexpressing lines showed more seedling vigor in response to salt stress in a germination assay compared to wild-type control plants. These results were confirmed when seedlings of ten new (2-component) lines overexpressing G1836 were re-examined in the current program. Six of the lines tested were more tolerant than wild type in a salt germination assay. Four of those lines were also more tolerant to sucrose and ABA in separate germination assays. Two of those four lines also were more tolerant than controls in a chilling growth assay.

Physiology (Soil Drought-Clay Pot) Summary. 35S::G1836 transformants exhibited an enhanced performance in a “whole pot” soil drought assay. Two (306 and 384) of three lines tested showed significantly improved survival compared to wild type. It should be noted that in an independent planting these lines showed no consistent difference to controls. In that experiment, however, the plants were dried down excessively (note very low soil drought scores); it could thus be the case that G1836 affords protection against moderate, but not severe drought stress.

TABLE 41
35S::G1836 drought assay results:

| | | Mean | Mean | p-value for | Mean | Mean | |
|---|---|---|---|---|---|---|---|
| | Project | drought | drought | drought score | survival for | survival for | p-value for difference |
| Line | Type | score line | score | difference | line | control | in survival |
| 306 | TCST | 0 | 0.37 | 0.28 | 0.060 | 0.063 | 0.89 |
| 306 | TCST | 4.7 | 1.0 | 0.0011* | 0.96 | 0.29 | 0.000000000011* |
| 384 | TCST | 0 | 0.37 | 0.28 | 0.036 | 0.063 | 0.33 |
| 384 | TCST | 2.5 | 1.0 | 0.018* | 0.61 | 0.29 | 0.0000057* |

TCST = two-components-supertransformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Many of the 35S::G1836 (2-component) lines obtained during the current study showed a complex variety of morphological changes including reduced overall size, abnormal leaf shape and coloration, vertically oriented leaves and alterations in flowering time. Additionally, some lines had reduced apical dominance, and floral abnormalities that resulted in poor fertility.

It should be emphasized that we have observed similar stress-tolerance phenotypes for several G481 related genes including G482, G485 and G1820. The comparable effects indicate that the genes are functionally related.

Potential applications. The results of this study strengthen our earlier conclusions that G481 and the related genes are excellent candidates for improvement of drought related stress tolerance in commercial species. Additionally, the results strongly implicate genes for the other subunits, such as G1836, in addition to the YBs (HAP3s), in conferring drought related stress tolerance. However, given some of the morphological off-types obtained from constitutive G1836 expression, it might be necessary to optimize the expression of the transgene by use of tissue specific or conditional promoters.

G2345 (SEQ ID NO: 21 and 22; Arabidopsis thaliana)—Constitutive 35S

Background. G2345 is a closely-related Arabidopsis homolog of G481, and is a member of the HAP3 (NF-YB) sub-group of the CCAAT-box binding transcription factor family. Based on phylogenetic and sequence analysis, the G2345 protein lies within the G481 (rather than the G482/G485) sub-clade and is most closely related to G1364. The aim of this study was to re-evaluate the effects of G2345 overexpression and determine whether the gene confers similar effects to G481.

Morphological Observations. We have now generated 35S lines for G2345 using the two component system; no consistent differences in morphology were observed compared to wild-type controls. It should be mentioned that a slight acceleration of flowering was noted in some of the lines, but that this was inconsistent across different plantings and could have depended on variables such as growth temperature. Some changes in leaf shape were also noted, but again, this effect was not consistent across lines.

Three batches of T1 lines were obtained. Some size variation was apparent in the second batch of plants, but plants from the other batches appeared wild type.

Four lines were examined in the T2 generation. T2-389 plants showed some size variation (small at early stages) and some individuals had short broad leaves. T2-390 plants appeared wild type on two plant dates but were slightly early flowering on a third plant date. T2-393 plants appeared wild type in a first planting but were marginally early flowering in two other plantings. T2-400 plants were grown on a single date and showed slightly early flowering and displayed short broad leaves.

Physiology (Plate assays) Results. Four 35S::G2345 lines were more tolerant than wild-type seedlings in a germination assay under cold conditions.

Physiology (Soil Drought-Clay Pot) Summary. Data from soil drought assays indicate that overexpression of G2345 can confer drought tolerance in Arabidopsis.

Four independent 35S::G2345 lines were examined:

Line 393 performed significantly better than wild type in two different plantings of a whole pot assay. Line 393 was tested on a third date but showed a wild type response on that date. It should be noted though, that on that date, the drought treatment was particularly severe, suggesting that this gene confers tolerance to moderate but not severe drought.

Line 389 also performed significantly better than controls in a “whole pot” assay in one of the assays.

TABLE 42
35S::G2345 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 389 | TCST | 1.0 | 0.22 | 0.069* | 0.18 | 0.063 | 0.011* |
| 389 | TCST | 1.1 | 0.67 | 0.15 | 0.26 | 0.20 | 0.19 |
| 393 | TCST | 2.3 | 0.22 | 0.0021* | 0.40 | 0.063 | 0.000000070* |
| 393 | TCST | 0.10 | 0.10 | 1.0 | 0 | 0.0071 | 0.34 |
| 393 | TCST | 0.58 | 1.0 | 0.15 | 0.15 | 0.26 | 0.12 |
| 393 | TCST | 0.40 | 0 | 0.078* | 0.036 | 0.014 | 0.27 |

TCST = two-components-supertransformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have now obtained three sets of 35S::G2345 lines using a two-component approach. These plants did not show any consistent alterations in morphology, although subtle effects on flowering time, overall size, and leaf shape were observed in some of the lines. However, 35S::G2345 lines did show positive results in physiology assays; a number of lines exhibited greater tolerance in a cold germination assay on plates and two lines were more tolerant in soil drought assays. Based on the stress tolerance effects obtained with G2345 overexpression, the protein has comparable activity to the G481 protein.

Potential applications. Given the results obtained thus far, G2345 may be used to confer tolerance to drought-related stress in commercial species.

G1248 (SEQ ID NO: 359 and 360; Arabidopsis thaliana)—Constitutive 35S

Background: G1248 represents a non-LEC1-like member of the HAP3 subfamily of CCAAT-box binding transcription factors, which based on our phylogenetic analysis, lies outside a clade containing the G481 and G482 groups. The aim of this study was to compare the effects of G1248, G481 and G482 overexpression and to determine whether proteins, related but outside the G481 and G482 clades, are capable of conferring abiotic stress tolerance.

Morphological Observations. We have now isolated and examined additional 35S::G1248 lines and found that overexpression of this gene produces complex effects on flowering time, plant size, growth rate and coloration.

Seventeen of 19 transformants were noted to be small and dark in coloration compared to controls. Four of 19 transformant lines, including line 339, were noted to be late developing. Two of 19 lines appeared wild type.

Six lines were examined in the T2 generation. In an initial planting three T2 lines were assessed. At early stages, T2-321 plants were noted to small with a few plants being dark in coloration. Similar phenotypes were seen at lower frequency in the T2 populations from two lines. Three further T2 populations were examined in a second planting. In this second planting, plants from all three of the T2 populations, including line 339, were small and slightly early developing compared to wild-type. This latter phenotype was similar to that reported in our initial genomics screens.

That the early developing phenotype was not equally apparent in different generations and planting dates demonstrates that such effects are complex and are likely to be heavily influenced by variables like temperature, light intensity, air flow, and transgene expression level which might have differed between lines and experiments.

Physiology (Plate assays) Results. Three of 10 lines tested were more tolerant than wild-type controls in a cold growth assay.

Physiology (Soil Drought-Clay Pot) Summary. One line, #339, showed significant evidence of greater drought tolerance than controls, including wild-type and CBF4-overexpressors.

TABLE 43
35S::G1248 drought assay results:

| | | | Mean | Mean | p-value for | Mean | Mean | p-value for |
|---|---|---|---|---|---|---|---|---|
| | Project | | drought | drought | drought | survival | survival for | difference |
| Line | Type | Control | score line | score control | score | for line | control | in survival |
| 339 | DPF | CBF4 OEX | 1.5 | 1.9 | 0.22 | 0.29 | 0.30 | 0.79 |
| 339 | DPF | CBF4 OEX | 1.8 | 1.5 | 0.52 | 0.23 | 0.11 | 0.013* |
| 339 | DPF | Wild type | 2.3 | 1.7 | 0.12 | 0.50 | 0.40 | 0.098* |
| 339 | DPF | Wild type | 1.8 | 0.90 | 0.015* | 0.30 | 0.16 | 0.0079* |

DPF = Direct promoter fusion
OEX = overexpressor
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion: During earlier genomics screens, 35S::G1248 lines exhibited accelerated flowering. We have now isolated additional 35S::G1248 lines and these exhibited rather complex changes in flowering time, plant size, growth rate and coloration. Many of the primary transformants were somewhat small, slow developing, and dark in coloration. Later, a number of T2 populations were morphologically examined and some of these showed accelerated flowering. It should be emphasized that the early developing phenotype was of variable penetrance and was therefore potentially influenced by variables like temperature, light intensity, air flow, and transgene expression level (which might have differed between lines and plantings).

Potential Applications: G1248 may be used to modify floral transition. The gene might also be used to confer tolerance to abiotic stress, in particular to cold or drought conditions.

G634 (SEQ ID NO: 49 and 50; Arabidopsis thaliana)—Constitutive 35S

Background. G634 (AT1G33240) was initially identified as two public partial cDNAs sequences (GTL1 and GTL2) which are splice variants of the same gene (Smalle et al, 1998). The published expression pattern shows that G634 is highly expressed in siliques and not expressed in leaves, stems, flowers or roots.

Three constructs were initially made for G634: P324, P1374 and P1717 contained a genomic clone of G634. P1374 and P1717 contain G634 cDNAs, with P1717 being the longer variant. Overexpression lines for P1717 were never analyzed during our genomics program. However lines for P324 showed some variable effects on size, but otherwise appeared wild type. Lines for P1374 exhibited an increase in trichome density on leaves and stems, but in other respects appeared wild type.

35S::G634 lines for P324 and P1374 showed a strong performance during our initial soil drought screens. Additionally, our array experiments on plants undergoing a soil-drought treatment indicated that G634 shows a small but significant up-regulation specifically in the recovery phase, following re-watering at the end of the drought.

35S::G634 lines also showed a shade tolerant phenotype.

This current project was initiated to analyze a greater number of G634 lines for stress tolerance phenotypes and to compare the effects of the different splice variants for G634 (see sequence section for details).

Morphological Observations. Additional sets of 35S::G634 lines have now been obtained for each of three different clones (see sequence section for details). Each of the clones produced an increase in trichome size/density when overexpressed.

Lines transformed with P1374 ere noted to have an increase in trichome density. The trichomes on these individuals also were larger than in wild type. Some of the lines were also rather late developing.

Lines generated with P324 were slightly small, with two lines showing slightly larger and more dense trichomes than controls. Two lines were slightly small and had an increase in trichome density/slightly larger trichomes relative to the control. The remaining lines appeared wild type.

Lines generated with P1374 were slightly small at early stages. A significant number showed increased trichome density. An increase in trichome size was noted in many of these lines.

Lines transformed with P1717 exhibited enlarged trichomes, and a majority of these had an apparent increase in trichome density. A substantial number of the lines were also markedly early flowering.

Physiology (Plate assays) Results. Three different PIDs for 35S::G634 were analyzed in abiotic stress assays. Overall, when all PIDs are combined, 9 out of 30 lines were more tolerant than wild-type seedlings in a plate based severe dehydration assay. Six out of 30 lines also had more root growth. When individual PIDs are considered, P1717 and P324 had 5 out of 10 lines and 3 out of 10 lines that were more tolerant than controls in the dehydration assay. P1717 and P1374 each had three out of 10 lines with more root growth than controls.

Physiology (Soil Drought-Clay Pot) Summary. In soil-based assays, most of the G634 overexpressing lines tested performed better than wild-type controls with regard to drought tolerance and recovery from drought treatment.

Discussion. We have now obtained lines for each of the three G634 overexpression clones, and in each case observed an increase in trichome density along with a potential increase in trichome size. Lines for the longest cDNA clone (P1717) also exhibited early flowering. Lines for each of the clones were tested in plate based assays and were more tolerant than wild-type in the severe dehydration assay. Lines for each of the two cDNA clones also showed more vigorous root growth than controls when grown on plates.

Potential applications. G634 has a wide range of potential applications including enhancing tolerance to various abiotic stresses, conferring shade tolerance, modulating flowering time, and modulating trichome structure/density, thus improving insect tolerance and accumulation of valuable secondary metabolites.

G1818 (SEQ ID NO: 403 and 404; Arabidopsis thaliana)—Constitutive 35S

Background. G1818 is an Arabidopsis gene which is a member of the HAP5 (NF-YC) subfamily of the CCAAT-box binding transcription factors. Since the NF-Y factors are known to act as trimeric complexes (comprising YA, YB, and YC subunits) genes for the other subunits (YCs and YAs) were tested to determine whether they confer drought tolerance in a comparable manner to genes encoding the YB subunit class to which G481 belongs. 35S::G1818 lines were examined during an earlier genomics screen and were found to be late flowering and have increased seed protein content. Additionally, 35S::G1818 lines gave positive results in a C/N sensing screen.

Morphological Observations. A number of G1818 overexpressing lines had upright, serrated leaves and were lighter green and late developing as compared with wild-type plants. Some lines showed no consistent differences relative to controls.

Physiology (Elate assays) Results. In the limited number of assays that have been performed thus far, G1818 overexpressors (3/10 lines) have been shown to confer greater tolerance to severe desiccation in plate based assays than wild-type controls.

Discussion. We have now obtained lines for G1818 overexpression clones. A number of lines were late developing and were distinct from wild type in that they were small, paler and later developing.

Potential applications. G1818 and related genes may be used to improve abiotic stress tolerance in plants, including drought related stress tolerance. These results implicate genes for the other subunits, such as G1818, in addition to the YBs (HAP3s), in conferring drought related stress tolerance.

G1820 (SEQ ID NO: 42 and 44; Arabidopsis thaliana)—Constitutive 35S

Background. G1820 is a member of the HAP5 (NF-YC) subfamily of the CCAAT-box binding transcription factors. Since the NF-Y factors are known to act as trimeric complexes (comprising YA, YB, and YC subunits) we are testing whether genes for the other subunits (YCs and YAs) can confer drought tolerance in a comparable manner to genes encoding the YB subunit class to which G481 belongs.

G1820 overexpression lines flowered earlier than controls. In physiology assays, these plants showed more tolerance to salt stress and ABA in separate germination assays. In a severe dehydration assay, 35S::G1820 seedlings were more tolerant compared to wild-type controls. The aim of this study was to re-assess the effects of G1820 overexpression on drought-related stress tolerance.

Morphological Observations. Many of the transformants showed a variety of morphological changes including reduced overall size, abnormal leaf shape and coloration (some lines were slightly yellow) and reduced apical dominance. A number of the lines also flowered earlier than controls. These phenotypic effects were generally more severe than those shown by 35S::G1820 direct fusion lines, suggesting that higher levels of G1820 activity might have been obtained using the 2-component system.

Physiology (Plate assays) Results. 35S::G1820 lines showed more tolerance to salt stress and insensitivity to ABA in separate germination assays. On a severe water deprivation assay, seedlings were more tolerant compared to wild-type controls.

A similar enhanced resistance to ABA was observed in eight of ten new 35S::G1820 (two-component) lines that were examined (no severe dehydration tolerance was observed however). In addition, these lines were more tolerant than wild-type to varying extents in several other assays including sucrose, salt, mannitol, and cold.

Physiology (Soil Drought-Clay Pot) Summary. 35S::G1820 lines showed a significantly enhanced performance before and after a period of drought as compared to wild type.

TABLE 44
35S::G1820 drought assay results.

| | | Mean | | p-value for | Mean | Mean | p-value for |
|---|---|---|---|---|---|---|---|
| | Project | drought | Mean drought | drought score | survival for | survival for | difference in |
| Line | Type | score line | score control | difference | line | control | survival |
| 2 | DPF | 2.0 | 0.71 | 0.37 | 0.27 | 0.17 | 0.017* |
| 3 | DPF | 3.0 | 1.7 | 0.21 | 0.58 | 0.24 | 0.023* |
| 5 | DPF | 3.0 | 1.7 | 0.066* | 0.36 | 0.24 | 0.082* |
| 7 | DPF | 3.0 | 1.7 | 0.21 | 0.69 | 0.24 | 0.0020* |
| 7 | DPF | 2.5 | 0.71 | 0.031* | 0.29 | 0.17 | 0.045* |
| 14 | DPF | 3.5 | 0.71 | 0.0016* | 0.37 | 0.17 | 0.000014* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Two-component 35S::G1820 lines have now been examined; a wide range of morphological alterations were observed, similar to those seen in our previous studies.

It should be emphasized that we have observed stress tolerance phenotypes for several other G481 related genes including G482, G485 and G1836. The similar effects seen when these genes are overexpressed strongly indicate that they are functionally related, at least with respect to the stress tolerance phenotype.

Potential applications. The results of this study strengthen our earlier conclusions that G481 and the related genes are excellent candidates for improvement of drought related stress tolerance in commercial species. Additionally, the results strongly implicate genes for the other subunits, such as G1820, in addition to the YBs (HAP3s), in conferring drought related stress tolerance. In addition to the effects on stress tolerance, G1820 could be used for manipulating flowering time; the gene may be particularly suitable in cases where an acceleration or induction of flowering is desired.

G1781 (SEQ ID NO: 55 and 56; Arabidopsis thaliana)—Constitutive 35S

Background. G1781 (SEQ ID NO: 56) represents a non-LEC1-like member of the HAP3 subfamily of CCAAT-box binding transcription factors, which based on our phylogenetic analysis of the Arabidopsis proteins, lies outside the clade containing the G481 and G482 groups. The aim of this study was to compare the effects of G1781, G481 and G482 overexpression and to determine whether proteins from outside the G481 and G482 clades are capable of conferring abiotic stress tolerance.

Morphological Observations. Overexpression of G1781 produced a number of developmental changes including accelerated flowering, dwarfing and rather spindly inflorescences.

Discussion. Previously, we observed that 35S::G1781 lines were early flowering, in a comparable manner to 35S::G482 lines. In the present study, we isolated a new batch of 35S::G1781 lines; again these lines showed early flowering, and some were markedly smaller than wild type. However, these lines exhibited a wild-type phenotype in plate based assays. 35S::G1781 lines were also tested in soil drought assays. One line did show a better performance than controls but the result was not obtained in a repeat experiment.

Potential application. Based on the results obtained to date, the clearest application for G1781 would be for modification of the floral transition. In particular, the gene may be suitable in cases where an acceleration or induction of flowering is desired.

G1334 (SEQ ID NO: 53 and 54; Arabidopsis thaliana)—Constitutive 35S

Background. G1334 is an Arabidopsis gene which is a member of the HAP2 (NF-YA) subfamily of the CCAAT-box binding transcription factors. Since the NF-Y factors are known to act as trimeric complexes (comprising YA, YB, and YC subunits) we are testing whether genes for the other subunits (YCs and YAs) can confer drought tolerance in a comparable manner to genes encoding the YB subunit class to which G481 belongs. 35S::G1334 lines were examined and were found to be small and dark in coloration. However, the transformants showed a wild-type response in all the physiology assays performed. The aim of this project is to analyze a greater number of lines for stress tolerance phenotypes.

Morphological Observations. A set of twenty new 35S::G1334 lines (#301-320) has been obtained. The majority of these transformants were small, with dark green compact rosettes and accelerated flowering versus wild type. Other lines were of wild-type size but still showed early flowering.

Discussion. A new set of 35S::G1334 has now been morphologically examined. These lines were early flowering, dark in coloration, and dwarfed relative to controls.

Potential applications. G1334 may be used to modify flowering time or plant development.

G2539 (SEQ ID NO: 407 and 408; Arabidopsis thaliana)—Constitutive 35S

Background. G2539 encodes an AP2 family protein and was identified as an upstream activator of G481.

Morphological Observations. 35S::G2539 lines were examined during earlier genomics screens and were found to small, dark in coloration, and have alterations in flowering time. Some lines flowered early, others late. Yet other lines had no differences in flowering time relative to controls.

Physiology (Plate assays) Results. Three of 10 lines were more tolerant than wild type in cold germination assays.

Potential applications. Based on the results obtained so far, G2539 may be used to enhance tolerance to abiotic stresses such as cold.

G3074 (SEQ ID NO: 409 and 410; Arabidopsis thaliana)—Constitutive 35S

Background. G3074 is an Arabidopsis gene which is a member of the HAP-like subfamily of the CCAAT-box binding transcription factors. Since the NF-Y factors are known to act as trimeric complexes we are testing whether genes for the other subunits can confer drought tolerance in a comparable manner to genes encoding the YB subunit class to which G481 belongs.

35S::G3074 lines were examined during earlier, limited genomics screen and showed a wild-type response in all assays. The most recent data were obtained project to analyze a greater number of lines for stress tolerance phenotypes.

Morphological Observations. At the seedling stage, some lines were small compared to controls. At later stages, the overexpressing lines generally were similar to wild-type in morphology and development.

Physiology (Plate assays) Results. Five of 10 overexpressing lines tested were more tolerant than wild type in plate-based severe desiccation assays.

Potential applications. Based on the results obtained so far, G3074 may be used to enhance tolerance to drought-related stresses in plants.

G3396 (SEQ ID NO: 41 and 42; Oryza sativa)—Constitutive 35S

Background. G3396 is an NF-YB gene from Oryza sativa and lies within the G481 sub-clade. G3396 corresponds to OsHAP3B and has been recently been shown to influence chloroplast biogenesis (Miyoshi et al., 2003). The aim of this study was to assess the role of this gene in drought stress-related tolerance, and to compare the effects with those of other G481-related genes.

Morphological Observations. 35S::G3396 lines exhibited a moderate delay in the onset of flowering (1-2 weeks under 24-hour light conditions) and produced rather dark leaves, which, at later stages, became enlarged and downward curled at the margins.

Physiology (Plate assays) Results. Five out of ten 35S::G3396 lines were more tolerant than wild-type seedlings in a cold germination assay. Three of these lines also performed better than wild-type on plates containing ABA.

Discussion. 35S::G3396 lines showed delayed flowering, a dark coloration, and produced leaves that became rather enlarged and curled, particularly at late stages. These phenotypes were somewhat comparable to those seen in 35S::G481 lines, indicating that the two proteins have similar activities. 35S::G3396 lines also showed positive results in plate assays and displayed more tolerance relative to controls in cold germination and were less sensitive to ABA in germination experiments. Some evidence of drought tolerance was detected in soil based assays under 24-hr light: two lines showed less severe stress symptoms than wild-type at the end of a drought period, but this effect was not consistently obtained between different plantings.

Potential applications. Based on the results obtained so far, G3396 has a similar activity to G481 and may be applied to enhance tolerance to abiotic stresses such as drought and cold. From the morphological phenotypes seen in 35S::G3396 lines, the gene could be applied to modify flowering time, leaf shape, or biomass. The dark coloration could be indicative of increased chlorophyll levels; thus G3396 might improve photosynthetic capacity and yield.

G3397 (SEQ ID NO: 35 and 36; Oryza sativa)—Constitutive 35S

Background. G3397 is an NF-YB gene from Oryza sativa and is phylogenetically more closely related to Arabidopsis G485/G482 than G481. G3397 corresponds to OsHAP3C and has been recently been shown to influence chloroplast biogenesis (Miyoshi et al., 2003). The aim of this study was to assess the role of G3397 in drought-related stress tolerance via overexpression, and compare the effects with that of the other G481-related genes.

Morphological Observations. 35S::G3397 lines exhibited a distinct acceleration in the onset of flowering (1-2 weeks under 24-hour light). 35S::G3397 lines also showed a reduction in overall size compared to controls. Such effects were also obtained in each of three T2 populations that were morphologically examined.

Physiology (Plate assays) Results. Four out of ten 35S::G3397 lines were more tolerant than wild-type seedlings in a cold germination assay. Additionally, seedlings of a number of the lines were somewhat larger and more vigorous than wild-type seedlings when grown on regular control plates without stress treatments.

Discussion. 35S::G3397 lines showed a very marked acceleration in flowering time, along with a reduction in overall plant size compared to wild type. A comparable phenotype has been obtained from overexpression of the two the most closely related Arabidopsis genes G485 and G482, indicating that G3397 has a similar activity to those proteins. 35S::G3397 lines have been tested in plate based abiotic stress assays; positive results were obtained in a cold germination assay. In particular, it is worth highlighting that 35S::G485 lines also were more tolerant in that assay, which further argues that G3397 has comparable activity to G485. Additionally, some of 35S::G3397 the lines showed enhanced seedling vigor compared to controls when grown on regular MS media without a stress treatment.

Potential applications. Based on the results so far obtained, G3397 may be applied to modify flowering time. The data from plate based assays indicate that the gene could be used to engineer abiotic stress resistance, and in particular, traits such as cold/wet germination.

G3398 (SEQ ID NO: 39 and 40; Oryza sativa)—Constitutive 35S

Background. G3398 is phylogenetically more closely related to Arabidopsis G485/G482 than G481. The aim of this study was to assess the role of G3398 in drought stress-related tolerance via overexpression, and compare the effects with those of the other G481-related genes.

Morphological Observations. 35S::G3398 lines exhibited a distinct acceleration in the onset of flowering (1-2 weeks under 24-hour light). Such effects were also seen in each of three T2 populations that were morphologically examined. 35S::G3398 lines exhibited a reduction in overall size compared to controls.

Physiology (Soil Drought—Clay Pot) Summary. 35S::G3398 lines showed a significantly enhanced performance in soil drought assays compared to wild type.

Three lines (#301, 303, and 304) showed significantly better performance than controls. On a later planting date, line 302 also showed significantly better survival than controls.

TABLE 45
35S::G3398 drought assay results:

| | | Mean | Mean | p-value for | Mean | Mean | |
|---|---|---|---|---|---|---|---|
| | Project | drought | drought | drought score | survival | survival for | p-value for difference in |
| Line | Type | score line | score | difference | for line | control | survival |
| 301 | DPF | 3.3 | 1.4 | 0.033* | 0.45 | 0.16 | 0.000000041* |
| 301 | DPF | 2.3 | 2.3 | 0.89 | 0.34 | 0.39 | 0.38 |
| 302 | DPF | 3.0 | 2.3 | 0.43 | 0.53 | 0.39 | 0.022* |
| 303 | DPF | 3.3 | 1.4 | 0.027* | 0.30 | 0.16 | 0.11 |
| 303 | DPF | 2.7 | 2.3 | 0.76 | 0.39 | 0.39 | 0.75 |
| 304 | DPF | 4.6 | 1.4 | 0.00091* | 0.58 | 0.16 | 0.0000000000000011* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3398 lines showed a 1-2 week acceleration in flowering time, compared to wild type. These early flowering lines were also markedly smaller than controls, and only small quantities of seed were obtained. As a result, only six lines were tested in plate-based physiology assays; no consistent difference in performance to controls was seen in those experiments. Nonetheless, 35S::G3398 lines did show enhanced tolerance in soil drought assays. The early flowering and drought tolerance phenotypes observed in 35S::G3398 lines were very similar to those seen in overexpression lines for G485 or G482, indicating that G3398 has comparable activity to those proteins.

Potential applications. Based on the results from these studies, G3398 could be applied to effect tolerance to drought-related stress. Additionally, G3398 could be used to modify flowering time; the gene may be particularly suitable in cases where an acceleration or induction of flowering is desired.

G3429 (SEQ ID NO: 57 and 58; Oryza sativa)—Constitutive 35S

Background. G3429 (SEQ ID NO: 57) is from Oryza sativa and was included in the drought program as a related gene to G481. From our phylogenetic analysis, G3429 is more distantly related to G481 than the other non-Arabidopsis genes and was included in this study as an example of an outlier. The gene encodes a protein corresponding to OsNF-YB 1 and has been shown to form a ternary complex with a MADS protein OsMADS18 (Masiero et al., 2002). The aim of this project was to assess the role of G3429 in drought stress-related tolerance, and to compare the effects with those of the other G481 related genes.

Morphological Observations. A significant number of 35S::G3429 lines exhibited a mild delay in the onset of flowering. Some lines exhibited late flowering and rather narrow leaves.

Physiology (Plate assays) Results. Six out of ten 35S::G3429 lines were more tolerant than wild-type seedlings in a germination assay in the presence of sodium chloride.

Discussion. Out of twenty 35S::G3429 T1 plants examined, six were notably late flowering and had narrow leaves compared to wild type. Plate-based stress assays revealed that 35S::G3429 lines had a marked enhancement in salt tolerance during germination relative to controls. The effects on flowering time and the plate assay results were somewhat similar to the results from overexpression of G481 and some of the G481-related proteins such as G3470. Thus, the G3429 protein could have influenced some of the same pathways which were acted on by those transcription factors.

Potential applications. Based on the results obtained to date, G3429 may be applied to effect abiotic stress tolerance, and in particular to enhance traits such as salinity tolerance. The gene might also be used to modify leaf development or to manipulate the floral transition, and could be of use in circumstances where a repression of reproductive growth is desired.

G3434 (SEQ ID NO: 11 and 12; Zea mays)—Constitutive 35S

Background. G3434 is an NF-YB gene from Zea mays and lies within the G481 sub-clade. G3434 is an ortholog of the rice protein, G3395. The aim of this study was to assess the role of G3434 in drought-related stress tolerance via overexpression, and compare the effects with that of the other NF-Y genes.

Morphological Observations. Overexpression of G3434 produced a moderate acceleration in the onset of flowering in many of the lines (about 2-5 days sooner than wild-type controls under continuous light conditions).

Physiology (Plate assays) Results. 35S::G3434 seedlings were more tolerant than wild-type seedlings in several abiotic stress assays. Out of eighteen total lines, nine, six, or four lines did better in germination assays where media contained sodium chloride, mannitol, or sucrose respectively. Seven out of eighteen lines did better in a severe plate based dehydration assay, and four of eighteen lines were more tolerant than wild type in a cold germination assay.

Physiology (Soil Drought-Clay Pot) Summary. Two lines of 35S::G3434 performed better than controls in terms of drought tolerance and recovery from drought in soil based assays.

Discussion. The majority of 35S::G3434 lines plants showed an acceleration in the onset of flowering. It should be noted that several other G481 homologs have been implicated in modulating flowering time indicating that G3434 has a similar activity. Interestingly, though, although G3434 lies within the same sub-clade as G481, the effects on flowering time were different; 35S::G481 lines were predominantly late flowering.

Potential applications. Based on the results obtained, G3434 has similar effects to other genes from the G481 study and may be used to enhance resistance to abiotic stresses such as cold, drought, and salinity. The gene might also be applied to regulate flowering time.

G3435 (SEQ ID NO: 29 and 30; Zea mays)—Constitutive 35S

Background. G3435 is an NF-YB gene from Zea mays and is phylogenetically more closely related to Arabidopsis G485/G482 than G481. The aim of this study was to assess the role of G3435 in drought stress-related tolerance via overexpression, and to compare the effects with those of the other NF-Y genes.

Morphological Observations. 35S::G3435 lines exhibited a distinct acceleration in the onset of flowering (1-2 weeks under 24-hour light). In general, the early flowering lines also accumulated less vegetative biomass than wild type. Equivalent effects on flowering time were also obtained in three T2 populations that were examined.

Physiology (Soil Drought-Clay Pot) Summary. 35S::G3435 lines showed a significantly better performance than controls in soil drought assays. Five of six lines tested out-performed wild-type in one or more runs of a “whole pot” soil drought assay.

TABLE 46
35S::G3435 drought assay results:

| | | | Mean | | | | |
|---|---|---|---|---|---|---|---|
| | | Mean | drought | p-value for | Mean | Mean | |
| | Project | drought | score | drought score | survival for | survival for | p-value for difference |
| Line | Type | score line | control | difference | line | control | in survival |
| 301 | DPF | 2.3 | 1.4 | 0.25 | 0.16 | 0.16 | 0.64 |
| 301 | DPF | 3.7 | 2.3 | 0.15 | 0.51 | 0.39 | 0.045* |
| 306 | DPF | 4.3 | 1.4 | 0.0057* | 0.51 | 0.16 | 0.0000000000082* |
| 306 | DPF | 1.3 | 2.3 | 0.33 | 0.40 | 0.39 | 0.94 |
| 308 | DPF | 2.3 | 1.4 | 0.38 | 0.33 | 0.16 | 0.000082* |
| 308 | DPF | 2.7 | 2.3 | 0.64 | 0.52 | 0.39 | 0.027* |
| 309 | DPF | 1.3 | 0.56 | 0.065* | 0.36 | 0.079 | 0.0000033* |
| 309 | DPF | 0.83 | 1.2 | 0.24 | 0.12 | 0.17 | 0.19 |
| 311 | DPF | 0.67 | 0.56 | 0.69 | 0.15 | 0.079 | 0.092* |
| 311 | DPF | 1.3 | 1.3 | 1.0 | 0.18 | 0.18 | 1.0 |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. The early flowering and drought tolerance phenotypes observed in 35S::G3435 lines were very similar to those seen in overexpression lines for G485 or G482, indicating that G3435 has comparable activity to those proteins. Surprisingly, 35S::G3435 lines showed a wild-type performance in most of the plate based assays, and in fact a small number of lines showed a worse performance in a heat growth assay. It is possible that these poorly performing lines represented events where the transgene was becoming silenced.

Potential applications. Based on the results from these studies, G3435 could be applied to effect tolerance to drought related stress. Additionally, G3435 could be used to modify flowering time; the gene may be particularly suitable in cases where an acceleration or induction of flowering is desired.

G3436 (SEQ ID NO: 33 and 34; Zea mays)—Constitutive 35S

Background. G3436 is an NF-YB gene from Zea mays and lies within the G482/G485 sub-clade. The aim of this study was to assess the role of G3436 in drought-related stress tolerance via overexpression, and to compare the effects with those of other NF-Y genes.

Morphological Observations. Overexpression of G3436 in Arabidopsis produced a striking acceleration in the onset of flowering (by approximately 1 week under 24-hour light conditions). In addition to the effects on flowering time, the lines also showed a reduction in vegetative biomass relative to controls. Such effects were observed in each of three batches of transformants, as detailed below. Three lines were examined in the T2 generation and comparable effects on flowering time and size were observed.

Physiology (Plate assays) Results. Six out of ten 35S::G3436 lines more tolerant than wild-type seedlings in a heat germination assay. Two of these lines also were more tolerant to cold germination or chilling growth assays than wild-type controls.

Discussion. It is noteworthy that such an acceleration of flowering was observed in G482 and G485 overexpression lines, indicating that G3436 has a related activity to those proteins. 35S::G3436 lines showed positive results in plate based abiotic stress experiments and were more tolerant of heat during germination compared to wild type. However, the results from soil drought experiments have so far been inconclusive. Data for particular lines were rather inconsistent between different runs of the experiments, suggesting that variables such as temperature, light intensity, and air-flow might influence the results.

Potential applications. Based on the results obtained so far, G3436 may be applied to effect abiotic stress tolerance, particularly to factors such as heat. The gene might also be applied to modify flowering time, and could be especially useful in circumstances where either an acceleration or induction of flowering is desired.

G3470 (SEQ ID NO: 3 and 4; Glycine max)—Constitutive 35S

Background. G3470 is an NF-YB gene from Glycine max and lies within the G481 sub-clade. The aim of this study was to assess the role of G3470 in drought stress-related tolerance via overexpression, and compare the effects with that of the other NF-Y genes.

Morphological Observations. 35S::G3470 lines exhibited a distinct delay in the onset of flowering (approximately one week under 24-hour light). Two different constructs (P21341 and P21471) were tested, and both produced similar effects on morphology. However, for unknown reasons, the penetrance of the late flowering phenotype was more apparent with P21341 than P21471. The constructs each contained cDNAs that encoded identical products, but there was a slight difference in the UTRs included in the constructs (see sequence section for details).

It should also be noted that the penetrance of the late flowering phenotype varied across lines and plant dates, suggesting that it might depend heavily on transgene expression level and/or environmental variables such as growth temperature and light intensity.

P21341 lines: Lines 301-320: 10/20 (#304, 307, 308, 309, 311, 316, 317, 318, 319, 320) displayed late flowering and exhibited slightly dark narrow leaves. The remaining lines appeared wild type.

P21471 lines: Lines 321-331: 1/11 (#327) showed delayed flowering. The rest appeared wild type.

Physiology (Plate assays) Results. 35S::G3470 lines for two different constructs (see sequence section for details) were tested in plate based physiology assays. Both constructs yield an enhanced tolerance in sodium chloride germination assays relative to controls, but P21471 lines showed enhanced tolerance in a number of additional assays, as detailed below.

P21341 lines. Seven (#302, 303, 305, 309, 310, 316, 318) often lines showed enhanced germination, relative to wild type, in NaCl germination assays. Two lines (301 and 303) showed enhanced tolerance in a heat growth assay.

P21471 Lines

4/10 lines (324, 329, 330, and 331) showed enhanced tolerance in NaCl germination assays.

5/10 (322, 326, 327, 330, 331) lines showed enhanced tolerance in mannitol germination assays.

5/10 (326, 327, 329, 330, 331) lines showed enhanced tolerance in sucrose germination assays.

4/10 lines (327, 329, 330, and 331) lines showed enhanced tolerance in ABA germination assays.

3/10 lines (324, 326, and 330) lines showed marginally enhanced tolerance in severe dehydration assays.

Physiology (Soil Drought-Clay Pot) Summary. A number of different 35S::G3470 lines for each of two different overexpression constructs (see sequence section for details) were tested. The results from these clay pot survival assays were somewhat inconclusive. In most of the plantings, 35S::G3470 lines showed a comparable performance to controls. A single line (#326) harboring construct (P21471) showed a significantly better performance than controls in a one of two runs of a whole pot assay, but exhibited a comparable performance in the second planting. A number of other lines performed worse than wild-type in one or more repeats of the assay.

Results from a separate study, however, indicate that G3470 confers an advantage under moderate drought stress conditions. In that study, individual plants from a 35S::G3470 line were grown in individual pots under 10-hour light conditions, water was withheld, and the proportion of the plants in the population showing moderate stress symptoms was recorded on consecutive days. In that experiment, wild-type plants showed stress symptoms sooner than those of the 35S::G3470 line.

TABLE 47
35S::G3470 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 326 | DPF | 1.5 | 0.60 | 0.016* | 0.25 | 0.11 | 0.0023* |
| 326 | DPF | 1.6 | 1.1 | 0.28 | 0.14 | 0.11 | 0.37 |

DPF = direct promoter fusion project
TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3370 lines exhibited similar phenotypes to 35S::G481 lines when overexpressed. Evidence of delayed flowering and a darker coloration was observed among the different lines, but these phenotypes were somewhat variable and potentially condition dependent. Additionally, 35S::G3470 lines showed a better performance in a number of plate based stress assays compared to controls. Occasional lines showed a better performance than wild-type in soil drought survival screens performed in clay pots under 24-hr light. 35S::G3470 plants were also tested in a more detailed drought study, where individual plants were droughted under 10-hr photoperiodic conditions in individual pots. In that study, 35S::G3470 plants showed less severe stress symptoms than controls on consecutive days of the early-mid part of the drought time course.

It should be noted that two different constructs for G3470 were tested (P21471 and P21341, see sequence section for details). While lines for these showed similar results in the soil drought clay pot screens, there was a difference in the penetrance of phenotypes seen in the morphology and plate based assays. Lines for P21341 showed higher penetrance of the late flowering effects but a lower penetrance of “hits” in plate based assays relative to P21471. The basis of this difference is unclear at present, since the two constructs encode identical G3470 proteins. Nonetheless, there was a slight difference in the UTR sequences included in the two constructs, which might have influenced the stability of the transcript.

All in all, 35S::G3470 lines showed similar morphological and drought-related stress phenotypes to 35S::G481 lines which strongly indicates that the two proteins have comparable activities.

Potential applications. Based on the enhanced performance of 35S::G3470 lines in abiotic stress assays, this gene could be used to confer tolerance to drought related stress.

Additionally, the delayed flowering and slightly dark coloration seen in the 35S::G3470 lines indicate that the gene might also be used to modify flowering time and enhance yield. A dark coloration could be due to increased chlorophyll or chloroplast content and may be indicative of an improvement in photosynthetic capacity.

G3471 (SEQ ID NO: 5 and 6; Glycine max)—Constitutive 35S

Background. G3471 is an NF-YB gene from Glycine max. Based on sequence alignments and phylogenetic analysis, G3471 lies within the G481 sub-clade. The aim of this study was to assess the role of G3471 in drought stress-related tolerance via overexpression, and to compare the effects with those of the other NF-Y genes.

Morphological Observations. Overexpression of G3471 produced alterations in leaf shape, coloration, and flowering time relative to controls. The predominant phenotype was delayed flowering and dark narrow leaves.

Physiology (Plate assays) Results. 35S::G3471 lines were tested in plate based physiology assays. Some of these transformants were more tolerant to sucrose (3 of 24 lines), less sensitive to ABA (3 of 24 lines), and severe desiccation (7 of 24 lines) than controls.

Physiology (Soil Drought-Clay Pot) Summary. 35S::G3471 lines showed evidence of drought tolerance in these clay pot screens. Each of three independent lines performed better than wild-type in one of two plantings.

TABLE 48
35S::G3471 drought assay results.

| | | Mean | Mean | p-value for | | | p-value for |
|---|---|---|---|---|---|---|---|
| | Project | drought | drought score | drought score | Mean survival | Mean survival | difference in |
| Line | Type | score line | control | difference | for line | for control | survival |
| 341 | DPF | 2.5 | 1.9 | 0.28 | 0.38 | 0.37 | 0.90 |
| 341 | DPF | 1.7 | 1.2 | 0.32 | 0.32 | 0.19 | 0.015* |
| 344 | DPF | 0.70 | 0.60 | 0.74 | 0.17 | 0.16 | 0.75 |
| 344 | DPF | 2.2 | 1.4 | 0.059* | 0.45 | 0.22 | 0.000067* |
| 347 | DPF | 1.1 | 1.2 | 0.93 | 0.41 | 0.36 | 0.39 |
| 347 | DPF | 2.0 | 1.0 | 0.031* | 0.38 | 0.16 | 0.000081* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Changes in flowering time were seen among the 35S::G3471 lines. A number of lines were slightly dark, had narrow leaves, and were late flowering. This effect was similar to that obtained with 35S::G481 lines, indicating that the two proteins have similar activities.

No consistent effects were seen in plate based assays, but each of three lines showed a better performance than wild-type in one of two runs of a soil drought assay.

Potential applications. Based on the results obtained, G3471 likely has similar applications to G481; the gene may be useful in conferring tolerance to drought-related stress. G3471 might also be applied to regulate developmental traits such as flowering time.

G3472 (SEQ ID NO: 31 and 32; Glycine max)—Constitutive 35S

Background. G3472 is an NF-YB gene from Glycine max and is phylogenetically more closely related to Arabidopsis G485/G482 than G481. The aim of this study was to assess the role of G3472 in drought-related stress tolerance via overexpression, and to compare the effects with those of the other G481 homologs.

Morphological Observations. A total of forty 35S::G3472 lines were obtained in two separate batches of T1 lines. With the exception of occasional plants that were slightly early flowering and had some size variation, neither of these sets of plants showed any consistent difference in morphology to controls. Three T2 populations were also examined, but overall, there were no consistent differences in morphology to wild type controls.

Physiology (Plate assays) Results. Three of ten 35S::G3472 lines showed an improvement in NaCl tolerance on germination relative to controls. Some of the lines also were rather more vigorous, had more extensively developed root systems and more root hairs than wild-type seedlings, when grown on regular MS control plates without a stress treatment.

Discussion. Although occasional plants showed slightly early flowering, overall, 35S::G3472 lines showed no clear-cut differences in growth and development compared to wild-type controls. These plants were tested in plate based assays: a small number of the lines showed mild NaCl tolerance in a germination assay and some others exhibited more extensive root development when grown on plates. In the other treatments, though, the plants showed a wild-type response, and no obvious improvement in tolerance was seen during soil based drought assays.

This gene did not produce accelerated flowering in the same manner as did other G482/G485 related genes such as G3474, G3475 and G3476 (SEQ ID NOs: 24, 16 and 20, respectively). It is possible therefore, that G3472 has weaker activity than some of the other proteins within the clade. Based on sequence alignments, it might be possible to predict particular key residues which are essential for the protein function.

Potential applications. Based on the plate data obtained so far, G3472 may be applied to improve abiotic stress tolerance traits such as salinity tolerance, or to enhance root development.

G3474 (SEQ ID NO: 23 and 24; Glycine max)—Constitutive 35S

Background. G3474 is an NF-YB gene from Glycine max and lies within the G482/G485 sub-clade. The aim of this study was to assess the role of G3474 in drought stress-related tolerance via overexpression, and to compare the effects with those of the other G481-related genes.

Morphological Observations. Overexpression of G3474 produced a marked acceleration in the onset of flowering (1-2 weeks under 24-hour light conditions) in many of the Arabidopsis transformants.

A significant number of lines displayed no consistent differences to wild type.

Early flowering was also observed in each of three T2 populations.

Discussion. 35S::G3474 lines showed accelerated flowering by 1-2 weeks compared to wild-type. This same phenotype was also noted for the many of the genes within the G482/G485 sub-clade, indicating that those proteins have similar activities. However, when 35S::G3474 lines were tested in plate based abiotic stress assays, no consistent difference in performance relative to controls was observed, suggesting that G3474 did not have a fully equivalent activity to G482/G485.

Potential applications. Based on the results obtained so far, G3474 could be applied to modify flowering time. In particular, the gene may be used in circumstances when an acceleration or induction of flowering is desired.

G3475 (SEQ ID NO: 15 and 16; Glycine max)—Constitutive 35S

Background. G3475 is an NF-YB gene from Glycine max and lies within the G482/G485 sub-clade. The aim of this study was to assess the role of G3475 in drought stress-related tolerance via overexpression, and to compare the effects with those of the other G481-related genes.

Morphological Observations. Overexpression of G3475 produced a very marked acceleration in the onset of flowering in Arabidopsis (approximately 1-2 weeks under 24-hour light conditions). Many of these plants also displayed a reduction in vegetative biomass compared to wild type.

Physiology results. Four of ten 35S::G3475 lines were more tolerant to cold than wild-type seedlings in a plate-based cold growth assay.

Discussion. 35S::G3475 lines showed accelerated flowering by ˜1-2 weeks compared to wild-type. This same phenotype was also noted for many of the genes within the G482/G485 sub-clade, indicating that those proteins have similar activities. When 35S::G3475 lines were tested in plate based abiotic stress assays, four of 10 lines showed enhanced tolerance in the cold growth assay relative to controls.

Potential applications. Based on the results obtained so far, G3475 could be applied to modify flowering time. In particular, the gene may be used in circumstances when an acceleration or induction of flowering is desired. The gene might also have a utility in conferring tolerance to abiotic stress; in particular to cold conditions.

G3476 (SEQ ID NO: 19 and 20; Glycine max)—Constitutive 35S

Background. G3476 is an NF-YB gene from Glycine max and lies within the G482/G485 sub-clade. The aim of this study was to assess the role of G3476 in drought stress-related tolerance via overexpression, and to compare the effects with those of the other G481-related genes.

Morphological Observations. Overexpression of G3476 produced an acceleration in the onset of flowering in Arabidopsis (by up to approximately 1 week under 24-hour light). These effects, however, were rather inconsistent between different batches of T1 plants, and a considerable number of plants showed no consistent differences to controls.

No alterations in flowering time were noted in three different T2 populations that were examined.

Physiology (Plate assays) Results. Three out of ten 35S::G3476 lines were more tolerant to cold in a germination assay. Three lines were also more tolerant to dehydration stress in a severe plate based drought assay.

Physiology (Soil Drought-Clay Pot) Summary. Two lines of 35S::G3476 transformants performed significantly better than wild-type in soil drought assays in at least one planting.

TABLE 49
35S::G3476 drought assay results:

| | | | Mean | | | | |
|---|---|---|---|---|---|---|---|
| | | Mean | drought | p-value for | Mean | Mean | |
| | Project | drought | score | drought score | survival for | survival for | p-value for difference |
| Line | Type | score line | control | difference | line | control | in survival |
| 309 | DPF | 1.3 | 0.60 | 0.12 | 0.24 | 0.13 | 0.022* |
| 309 | DPF | 2.9 | 0.80 | 0.00014* | 0.58 | 0.11 | 0.000000000014* |
| 321 | DPF | 1.2 | 0.40 | 0.026* | 0.24 | 0.079 | 0.00055* |
| 321 | DPF | 0.30 | 0.40 | 0.68 | 0.064 | 0.060 | 0.83 |

DPF = direct promoter fusion project
TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3476 lines showed accelerated flowering by 1 week compared to wild-type. However, this effect was of variable penetrance between lines and plantings suggesting that it might be dependent on a specific range of transgene expression and/or growth conditions. It should be noted that many of the genes within the G482/G485 sub-clade produced early flowering when overexpressed, indicating that those proteins have similar activities. 35S::G3476 lines showed encouraging results in abiotic stress assays.

Potential applications. Based on the results obtained, G3476 could be applied to effect tolerance to abiotic stresses such as drought and cold conditions. The gene might also have a utility in modification of flowering time.

G3478 (SEQ ID NO: 25 and 26; Glycine max)—Constitutive 35S

Background. G3478 is an NF-YB gene from Glycine max and lies within the G482/G485 sub-clade. The aim of this study was to assess the role of G3478 in drought stress-related tolerance via overexpression, and compare the effects with those of the other G481-related genes.

Morphological Observations. Overexpression of G3478 accelerated the onset of flowering in Arabidopsis. The transformants tended to have spindly stems.

Discussion. 35S::G3478 lines showed accelerated flowering time, by 1-2 weeks and were slightly small compared to wild-type. The same flowering phenotype was also noted for the many of the genes within the G482/G485 sub-clade, indicating that these proteins have similar activities.

Potential applications. Based on the results obtained so far, G3478 could be applied to modify flowering time. In particular, the gene may be used in circumstances when an acceleration or induction of flowering is desired.

G3876 (SEQ ID NO: 7 and 8; Oryza sativa)—Constitutive 35S

Background. G3876 is a maize gene which is a member of the HAP3 (NF-YB) subfamily of the CCAAT-box binding transcription factors. In phylogenetic analyses, the protein lies within the same sub-clade as G481.

Morphological Observations. Two sets of 35S::G3876 lines have been selected. No clear-cut alterations in morphology were noted, although a few of the lines were noted to have slight changes in flowering time. Line 302 was slightly late flowering, whereas #305 and #311 were slightly early flowering. Fifteen other lines appeared wild type in morphology and development.

Physiology (Plate assays) Results. Six of 10 lines tested were more tolerant to cold during germination than wild type, and 4 of 10 lines were more tolerant to desiccation in plate-based assays.

Discussion. A minority of 35S::G3876 lines showed slight alterations in flowering time. Perhaps more significantly, improvements in cold germination and desiccation tolerance were noted in plants that had wild-type development and morphology.

Potential applications. Based on the results obtained so far, G3876 could be applied to modify flowering time. The gene may also be used to improve drought and cold tolerance without causing undesirable morphological or developmental defects.

G481 (SEQ ID NO: 1 and 2; Arabidopsis thaliana)—Double Overexpression

Background. The aim of this double overexpression approach was to determine whether different leads gave an additive effect on drought/disease/low N tolerance when “stacked” together in the same line. A crossing strategy was initiated to construct the lines listed below.

Morphological Observations.

(1) 35S::G481×35S::G1073 (SEQ ID NO: 113)

A doubly homozygous line has been obtained. These plants showed an additive phenotype compared to the two parental lines. The double overexpressors tended to be late flowering, had larger rosettes than controls (particularly at late stages of growth), with somewhat enlarged and curled leaves. These plants also tended to be darker green than controls.

(2) 35S::G481×35S::G867 (SEQ ID NO: 87)

The F1 plants from this cross were dark in coloration, showed narrow leaves, and were distinctly late flowering. Such phenotypes were perhaps stronger than those seen in the 35S::G481 parental line.

(3) 35S::G481×35S::G682 (SEQ ID NO: 59)

These plants showed an additive phenotype between G682 and G481 overexpression and were small at early stages, glabrous and late flowering.

(4) 35S::G481×35S::G489 (SEQ ID NO: 45)

The double overexpression line showed a comparable phenotype to the 35S::G481 parental line: the plants were late flowering, dark in coloration, and had rather narrow leaves.

(5) 35S::G481×35S::G1792 (SEQ ID NO: 221)

These F1 plants showed a wild-type phenotype, and unexpectedly, did not show a delay in flowering.

(6) 35S::G481 (female)×35S::G3086 (SEQ ID NO: 291)(male)

Twenty F1 plants were obtained. All showed an identical phenotype to the 35S::G3086 parental line: very early flowering, reduced size, and spindly inflorescences.

Physiology (Plate assays) Results. Six out of ten double overexpressing lines for 35S::G1073 supertransformed into a 35S::G481 line were more tolerant to cold conditions in a plate-based germination assay. Four lines also performed better than control seedlings in a root growth assay under low N.

Physiology (Soil Drought-Clay Pot) Summary. 35S::G481×35S::G489 and 35S::G481×35S::G1073 lines were more drought tolerant than controls in clay pot screens.

TABLE 50
35S::G481 X 35S::G489 drought assay results

| | | Mean | Mean | p-value for | Mean | Mean | p-value for |
|---|---|---|---|---|---|---|---|
| | | drought | drought score | drought score | survival | survival | difference in |
| Line | Project Type | score line | control | difference | for line | for control | survival |
| F1-16-8 | G481 x G489 | 2.6 | 2.0 | 0.053* | 0.46 | 0.31 | 0.0081* |
| | Double OEX | | | | | | |
| F1-16-8 | G481 x G489 | 2.6 | 2.0 | 0.26 | 0.49 | 0.36 | 0.030* |
| | Double OEX | | | | | | |
| F1-1-46 | G481 x G1073 | 3.4 | 2.3 | 0.021* | 0.57 | 0.36 | 0.00070* |
| | Double OEX | | | | | | |
| F1-1-46 | G481 x G1073 | 2.4 | 1.8 | 0.10* | 0.57 | 0.31 | 0.000011* |
| | Double OEX | | | | | | |

Double OEX = double overexpression resulting from crossing of two homozygous lines
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion: A crossing strategy was initiated to construct these lines; details of progress are shown in the morphology section. Double homozygous lines have been obtained between 35S::G481 and 35S::G682, 35S::G1073 and 35S::G489.

The 35S::G481;35S::G1073 double overexpression line has also been examined in a single pot soil drought assay at well-watered, mild drought, and moderate drought states for a variety of physiological parameters. Based on HPLC measurements, the double showed higher chlorophyll and carotenoid levels at the two drought states than wild-type. A significantly higher level of proline was also seen in the 35S::G481;35S::G1073 line versus wild-type at both drought states. A higher level of ABA versus wild-type was also apparent at a mild-drought state.

The 35S::G481×35S::G3086 combination produced an interesting morphological phenotype. F1 plants have recently been obtained, and these all showed a 35S::G3086-like morphology; the plants were very early flowering.

Potential applications: The 35S::G481;35S::G489 combination may be used to confer drought tolerance in plants.

The 35S::G481;35S::G3086 combination might have an application in soybean. 35S::G481 in soybean produced a delayed flowering off-type that is associated with a yield penalty. Combining G481 with G3086 overexpression in the same soy line may afford drought tolerance without the delayed flowering caused by G481 alone.

### The G682 Clade

G682 (SEQ ID NO: 59 and 60; Arabidopsis thaliana)—Constitutive 35S

Background. G682 was selected for the drought program based on the enhanced tolerance of 35S::G682 lines to drought-related stresses such as heat. A genetic analysis of G682 function has not yet been published, but its sequence (AX366159) has been included in patent application WO0208411.

Previously, we observed that G682 overexpression produced enhanced tolerance heat during germination. The aim of this study was to re-assess a greater number of 35S::G682 lines and to allow comparison of the G682 overexpression effects to those of its paralogs and orthologs. We also sought to test whether use of a two-component overexpression system would produce any strengthening of the phenotype relative to the use of a 35S direct promoter-fusion. The effects of three different clones were tested in the two-component system: two distinct cDNA clones and a genomic clone.

Two additional sets of direct promoter fusion lines were also examined in this study. One set contained a genomic clone of G682 in the kojak background. The other set contained the same genomic clone in a wild-type background. The kojak mutant produces only vestigial root hairs, and thus the purpose of overexpressing G682 in this background was to test whether the effects of G682 were dependent on increased root hairs.

Morphological Observations. Overexpression of G682 produced a spectrum of effects on Arabidopsis morphology including a glabrous phenotype, reduced pigmentation levels, alterations in flowering time, and increases in root hair density. Most of plants were reduced in size.

Two component lines exhibited a comparable glabrous phenotype to the G682 overexpression lines using a 35S direct promoter fusion construct.

The glabrous effects were highly penetrant: 16/20 T1 plants were completely glabrous, 3/20 T1 plants were partially glabrous (305, 306, 309) and 1/20 (#319) showed a wild-type trichome density. It should be noted that a number of lines #301, 304, 311, 315, 316, 318 were also observed to be smaller than wild type. No difference in coloration compared to wild-type was noted in the seed from this set of plants.

Trichome distribution on lines 301-316 were examined once again in the T2 generation: from lines 305, 306, 309 again were partially glabrous, while plants from all the other lines were completely glabrous.

Three lines were examined again in the T3 generation: T3-306 showed a partial glabrous phenotype and was slightly early flowering. Lines T3-307 and T3-310 exhibited a completely glabrous phenotype (of the lines submitted for physiological assays, the following showed a segregation on selection plates in the T2 generation that was compatible with the transgene being present at a single locus: 301, 302, 306, 308, 309, 310, 312, and 314. Lines 305 and 307 showed segregation that was compatible with insertions at multiple loci.).

Lines 2061-2070 (contained P23516, a cDNA variant clone of G682, see sequence section for details):

All were slightly small, 1/10 died at early stages, 3/10 were completely glabrous (#2063, 2069, 2070), 3/10 (2061, 2062, 2067) were partially glabrous and 3/10 (#2064, 2065, 2066) appeared wild type.

Lines 2081-2092 (contained P23517, a G682 cDNA clone, see sequence section for details):

All slightly small at early stages. 2/12 (#2085, 2092) were partially glabrous, 1/12 (#2089) appeared wild type. Remaining 8/12 plants were completely glabrous.

The higher frequency glabrous phenotype obtained with the lines containing P23517 suggests that the G682 protein encoded by that cDNA might be more potent than the one encoded by P23516.

Direct promoter-fusion lines, as compared to controls

Two new sets of 35S::G682 direct promoter fusion lines have recently been obtained, containing our original genomic clone P108. Lines 1761-1780 were derived from transformation of that construct into a kojak mutant background, whereas the lines 1781-1800 were derived from transformation into a wild-type Columbia background.

Lines 1761-1780 (contained a genomic clone of G682 in the kojak background):

At early stages, all were pale and glabrous. Later, 2/20 plants appeared wild type (#1777, 1778) whereas the others showed a glabrous or partial glabrous phenotype. Most of plants were reduced in size. A number of lines were early flowering: 1768, 1771, 1779 were very early, whereas #1761-1764, 1773-1775 were slightly early flowering. No difference in coloration compared to wild-type was noted in the seed from this set of plants.

Kojak is a mutant in the cellulose synthase like gene CSLD3 (At3g03050) which produces only vestigial root hairs. The aim of this experiment was to test whether the 35S::G682 enhanced stress resistance phenotype was dependent on increased root hair density. The intention was that we would be able to test the effects of G682 in the absence of an ability to produce root hairs. Very surprisingly, however, the 35S::G682 phenotype was epistatic to the kojak mutation (see physiology plate results) and the 35S::G682;kojak lines exhibited root hairs. Such as result suggests that G682 overexpression compensated for the kojak defect.

Lines 1781-1800 (contained a genomic clone of G682) in the wild type Background.

At early stages, all were pale and glabrous. Later, 19/20 were either glabrous or partial glabrous (#1789 appeared wild type). All others were glabrous and slightly small. Some plants (particularly #1791) flowered early. A number of the lines were rather slow developing versus wild-type: 1784-1787, 1795, and 1796. No difference in coloration compared to wild-type was noted in the seed from this set of plants.

Epidermal patterning in 35S::G682, Line 16: To preliminarily determine if G682 overexpression caused changes in stomatal density, we observed epidermal peels of 35S::G682 (line 16) and controls, less than one week after bolting began. Epidermal density was equivalent in mature rosette leaves (both abaxial and adaxial surfaces) and in the inflorescence stem. On the abaxial (lower) side of expanding cauline leaves, however, epidermal density was somewhat greater in wild-type plants (G682 OE plants had approximately one-third less stomata per unit area).

Physiology (Plate assays) Results. We previously observed that G682 overexpressors were more tolerant to heat during germination. The plants were glabrous with tufts of increased root hair density compared to wild type.

Enhanced abiotic stress tolerance has now been confirmed using ten 2-component 35S::G682 lines (301 to 314). All ten lines showed increased tolerance to sucrose on germination and also were more tolerant than wild type to varying extents in at least one or more of the following germination assays: sodium chloride, mannitol, heat, and ABA. Nine of the lines also showed a marked increase in root hair density and were glabrous. 35S::G682 lines performed better in the C:N sensing and growth under low nitrogen assays.

In contrast, 35S::G682 direct promoter fusion lines (1781 to 1798) had less dramatic phenotypes. In most stress assays, no increased tolerance was observed., Some lines did show an increase in root hair density and performed better in the C:N-sensing and growth-under-low-nitrogen assays. The difference in the phenotypes between the two-component and direct-promoter-fusion lines probably reflects greater expression in the two-component lines.

35S::G682 seedlings in the kojak background (lines 1761 to 1780) were also analyzed in physiological assays in an attempt to see how important the presence of root hairs are for the stress tolerance phenotypes observed with G682 overexpression. 35S::G682 seedlings in a kojak background performed well in a C:N sensing assay. Two of these ten lines performed well in a root growth assay under low nitrogen; the seedlings were more vigorous and had more extensively developed roots than controls. Some of the plants also did well in heat germination and heat growth assays.

As noted in the morphology section, the overexpression of G682 rescued the vestigial-root phenotype of the kojak mutant. This rescue was not fully penetrant. The rescue of the kojak phenotype precluded our attempt to determine the degree to which root hairs are necessary for the stress resistance phenotypes seen in G682 OE lines.

Discussion. 35S::G682 two-component lines showed a strong glabrous phenotype, similar to what was observed during our initial genomics program. Additionally, a number of the lines were noted to be smaller than controls, an effect that had not been previously recognized.

A high penetrance of the glabrous phenotype was seen with one of the cDNA variant clones (P23517), and with the G682 genomic clone. The other cDNA clone (P23516) had lower penetrance. 35S::G682 two component lines were typically smaller than wild-type.

Surprisingly, over-expression of G682 in the kojak background rescued the phenotype of the mutant. The rescue of the kojak phenotype by G682 overexpression precluded our attempts to determine the importance of root hairs in the stress tolerance phenotypes conferred by G682.

Direct promoter fusion lines had weaker phenotypes compared to the two-component lines. The most striking differences between the direct fusion lines and the two-component lines were seen in the NaCl germination assay, and in the sucrose germination assay where the two-component lines gave significant stress tolerance. The direct fusion lines did not show increased tolerance in these assays.

The performance of 35S::G682 two-component lines in the clay-pot soil drought assay was outstanding, as they consistently performed better than wild-type controls. All three two-component lines tested showed increased survivability in two separate experiments. Direct fusion lines also showed some evidence of drought tolerance, but this was less marked than with the two-component lines.

Thus, we have obtained substantially stronger phenotypes with 35S::G682 two-component lines than direct fusion lines. This might be attributable to higher levels of G682 expression in the former.

Three independent 35S::G682 lines (a pair of direct fusion and a two-component line) were tested in “single pot” soil drought assays, in which a number of different physiological parameters were measured. A general reduction in chlorophyll levels was noted, and in some cases, a reduction in the rate of photosynthesis was seen. These parameters correlate with the fact that 35S::G682 lines were markedly dwarfed and rather yellow in coloration. It should also be noted that many of the plants in these experiments were too small to used for physiological measurements.

Potential applications. The results of these overexpression studies confirm that G682 is an excellent candidate gene to modify trichome or root hair development, and for improvement of drought-related stress or nutrient limitation tolerance in plants. However, the slight decrease in size seen in some of the lines, suggests that the gene might require optimization by use of different promoters or protein modifications, prior to product development.

G682 (SEQ ID NO: 59 and 60; Arabidopsis thaliana)—Vascular SUC2

Background. The aim of this project was to determine whether expression of G682 from a SUC2 promoter, which predominantly drives expression in a vascular pattern, was sufficient to confer stress tolerance that is similar to, or better than, that seen in 35S::G682 lines.

Additionally, this study allowed us to assess whether use of an alternative promoter could eliminate some of the undesirable size reductions that are associated with G682 overexpression (see 35S::G682 report), while still conferring enhanced stress tolerance.

Morphological Observations. For plants transformed with P21525, which contains a SUC2::G682 direct promoter-fusion, changes in trichome distribution were apparent in 8/12 T1 plants. These individuals all exhibited a partial glabrous phenotype in which trichomes were absent from the central portions of the leaves nearest the mid-vein, but became present towards the leaf margins. Some slight variation in flowering time was also noted in this population, but in other respects, the lines were of a wild-type size and morphology.

Three lines were examined in the T2 generation. All of these populations showed a partial glabrous phenotype comparable to that seen in the T1 generation. One line also showed accelerated flowering. Plants from each of the populations were noted to slightly small.

Initially, three sets of 2-component lines were obtained for which an opLexA::G682 construct was supertransformed into a SUC2::LexA-GAL4TA promoter driver line. Considerable size variation was apparent among these plants in the T1 generation, but no effects on trichome development were noted. However, the GFP reporter in these supertransformants indicated that activity from the SUC2 promoter was becoming silenced in subsequent generations. The lines were therefore not submitted for physiological assays.

Later we obtained 2-component lines in an alternative SUC2 promoter line created by supertransformation with two different opLexA::G682 constructs (P23517 and P23516). In this line the GFP reporter indicated that the SUC2::LexA driver was active, but none of the resulting lines produced alterations in trichome distribution. However, some of the lines did show effects on flowering time. Both P23517 and P23516 were functional and produced a glabrous phenotype when supertransformed into a 35S driver line, see 35S::G682 results, above.

At present the basis of the difference in results obtained between the one and two component SUC2::G682 lines is unclear, but it could indicate that a particular range of expression is needed to produce the glabrous effects.

Physiology (Plate assays) Results. Six of ten SUC2::G682 direct promoter fusion lines were larger than control seedlings in a heat germination assay. Three lines also did well in a heat growth assay. These seedlings also were somewhat larger and more vigorous on control plates in the absence of a stress treatment. In the other assays, the plants were not significantly different from wild-type.

A set of ten two-component lines were tested later. Three of these lines showed a weak positive result in a chilling growth assay, but in the other assays, a wild-type response was obtained.

Interestingly, SUC2::G682 lines did not show consistently better results than controls in the N assays, indicating that a vascular specific pattern of expression was not sufficient to confer tolerance to such conditions. The SUC2::G682 lines were also not noted to have any increase in root hair density

Physiology (Soil Drought—Clay Pot) Summary. Three independent SUC2::G682 lines were tested in soil drought assays. One line (#1542) showed significantly better survival than controls on two of three plant dates. This line showed a wild-type performance when tested on a third plant date.

TABLE 51
SUC2::G682 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 1542 | DPF | 3.2 | 1.6 | 0.0012* | 0.48 | 0.24 | 0.000031* |
| 1542 | DPF | 0.60 | 0.20 | 0.16 | 0.14 | 0.050 | 0.012* |
| 1542 | DPF | 1.0 | 0.90 | 0.77 | 0.22 | 0.20 | 0.74 |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. SUC2::G682 lines containing a direct promoter-fusion construct have been established, as well as SUC2::G682 two component lines. A partial glabrous phenotype was observed in the majority of the direct promoter-fusion lines: leaves of SUC2::G682 plants were generally devoid of trichomes in the central region nearest the mid-vein, but developed those structures towards the margins of the leaves. We have previously established that G682 acts to repress trichome formation and the above trichome distribution correlated well with the expression pattern produced by the SUC2 promoter. G682 protein (or signaling molecules associated with its activity) would likely have been present at the highest levels near the mid-vein of leaves and could have moved from those regions to inhibit trichome specification in the adjacent epidermal tissue. In other respects, SUC2::G682 direct promoter-fusion plants were morphologically wild type. Some size reduction was observed in the SUC2::G682 T2 generation plants, but this was rather less marked than that seen in 35S::G682 lines.

Two-component lines, surprisingly, did not show a glabrous phenotype. Two different promoter background lines, and three different opLexA::G682 constructs were used. It is worth noting that the opLexA::G682 constructs used in this study produced glabrous phenotypes in combination with a 35S::LexA-GAI4IA construct. (see 35S::G682 section). It is currently unclear why the two component SUC2 lines did not produce a partial glabrous phenotype.

Two-component lines were not tested in soil assays. These lines were subjected to plate based assays, but showed a wild-type response, apart from a weak positive result in a chilling assay.

Potential applications. Overexpression studies indicate that G682 is an excellent candidate for improvement of drought related stress tolerance in commercial species. The results of this SUC2 experiment indicate that G682 can confer some stress tolerance when expressed under the control of a vascular promoter. Although the tolerance seen was less compelling than in 35S lines, dwarfing off-types seen in 35S::G682 lines were less apparent in the SUC2::G682 lines. Thus, a vascular expression pattern may be useful for optimization of this polynucleotide in crops.

G682 (SEQ ID NO: 59 and 60; Arabidopsis thaliana)—Epidermal CUT1

Background. The aim of this project was to determine whether expression of G682 from a CUT1 promoter (which predominantly drives expression in the shoot epidermis, and results in high level expression in guard cells), was sufficient to confer stress tolerance that is similar to, or better than, that seen in 35S::G682 lines.

Additionally this study allowed us to assess whether use of an alternative promoter could eliminate some of the undesirable size reductions that are associated with G682 overexpression (see 35S::G682 report), while still conferring enhanced stress tolerance.

Morphological Observations. Arabidopsis lines in which G682 was expressed from the CUT1 promoter (using the two component system) exhibited no consistent differences in growth and development compared to controls. Two batches of CUT1::G682 lines were obtained; some size variation was observed among the T1 plants, but overall, their morphology appeared wild type. Three T2 populations were also examined and exhibited wild-type morphology.

Physiology (Plate assays) Results. Six out of ten CUT1::G682 lines were more tolerant to NaCl than wild type in a germination assay. One line was substantially more tolerant to cold than controls in a germination assay.

Physiology (Soil Drought-Clay Pot) Summary. Three of eight CUT1::G682 lines tested were more tolerant to drought in a soil based drought assay than controls.

Discussion. We have produced CUT1::G682 lines using the two component system. These lines showed no consistent differences to wild-type and interestingly, did not show a glabrous phenotype. This could indicate either that CUT1 did not produce high enough levels of G682 activity in the epidermis to repress trichome initiation, or that activity of G682 is required in sub-epidermal layers to cause that effect.

CUT1::G682 lines have now been subjected to plate based physiology assays. Six of ten lines tested produced a moderate enhancement of tolerance to sodium chloride on germination plates. Although this was a somewhat weaker phenotype than that shown by 35S::G682 lines, it was of interest since the CUT1 promoter does not drive significant expression in the root, and the CUT1::G682 lines were not observed to show any increase in root hair density. Therefore, the stress resistance phenotype of plants with increased G682 activity is separable from changes in root hair number. The increased NaCl stress tolerance seen in CUT1::G682 lines appears to have arisen from increased levels of G682 in the shoot epidermis. However, there is the possibility that G682 protein, or signals associated with its activity, were able to move from the epidermal cell layer to other regions of the shoot.

In clay-pot soil assays, one line of CUT1::G682 overexpressors showed statistically significant drought tolerance relative to wild-type controls.

Potential application. Overexpression studies indicate that G682 is an excellent candidate for improvement of drought related stress tolerance in commercial species. The results of this CUT1 experiment indicate that G682 can confer some drought-related stress tolerance independently of an increase in root hair density; thus the gene could be applicable to plant species in which the roots already differentiate a maximum number of root hairs.

G682 (SEQ ID NO: 59 and 60; Arabidopsis thaliana)—Epidermal LTP1

The aim of this project was to determine whether expression of G682 from a LTP1 promoter (which predominantly drives expression in the shoot epidermis, and results in particularly high levels of expression in trichomes), was sufficient to confer stress tolerance that is similar to, or better than, that seen in 35S::G682 lines.

Additionally this study allowed us to assess whether use of the LTP1 promoter could eliminate the undesirable size reduction associated with G682 overexpression, while still conferring enhanced stress tolerance.

Morphological Observations. Two sets of lines were obtained in which G682 was expressed from the LTP1 promoter. Lines 1721-1736 contained a direct fusion construct (P23328): many of these lines showed a glabrous phenotype comparable to that seen in 35S::G682 lines. Additionally, most of the lines were slightly small compared to wild type. Of 16 lines obtained, 8/16 were totally glabrous. Two of 16 lines were partially glabrous. The remaining T1 plants appeared wild type. Three lines were examined in the T2 generation. Two lines were small and glabrous, whereas the one population were partially glabrous.

A set of LTP1::G682 2-component lines were subsequently obtained. Surprisingly, none of these lines exhibited a glabrous phenotype. The basis for this difference is not clear, but it should be noted that the opLexA::G682 construct (P23517) was transformed into a 35S driver line, and the majority of lines were glabrous. It is possible that there is an optimum range of expression needed to generate glabrous effects with LTP1 and that this was only obtained with the direct fusion arrangement.

Physiology (Plate assays) Results. Three out of ten LTP1::G682 lines performed better than controls in the C:N sensing and growth under low nitrogen assays. Seedlings were also glabrous and were somewhat larger. The greater tolerance of 6 of 10 lines tested relative to controls seen in the growth assay under chilling conditions might reflect the somewhat larger size and lack of anthocyanin production in LTP1::G682 plants.

Discussion. We have produced both LTP1::G682 direct promoter-fusion, and LTP1::G682 two-component lines. Surprisingly, the direct promoter-fusion lines were typically glabrous, whereas the two-component lines were not. The opLexA::G682 construct used in this study produced a glabrous phenotype in combination with a 35S::LexA-GAL4TA construct (see 35S::G682 section).

Ten of the LTP1::G682 direct promoter-fusion lines have been subjected to plate based physiology assays. Three out of the ten lines performed better in the C:N sensing and growth-under-low-nitrogen assays. For unknown reasons, in clay-pot soil drought assays, LTP1::G682 lines performed significantly worse than wild-type.

Potential applications. The results of this LTP1 experiment indicate that G682 can confer some stress tolerance independently of an increase in root hair density; thus the gene could be applicable to plant species in which the roots already differentiate a maximum number of root hairs. Soil-drought assays, however, indicate that LTP1::G682 is not an optimal combination for conferring drought tolerance in soil-grown plants.

G682 (SEQ ID NO: 59 and 60; Arabidopsis thaliana)—Super Activation (N-GAL4-TA)

Background. G682 was included in the drought program based on the increased tolerance of 35S::G682 lines to drought-related stresses. The aim of this project was to determine whether the efficacy of the G682 protein could be improved by addition of an artificial GAL4 activation domain.

Morphological Observations. Lines containing a 35S::GAL4-G682 construct exhibited no consistent differences in morphology to wild type controls. Some size variation was noted, though, among two batches of T1 lines. Interestingly, no evidence of a glabrous phenotype was evident. Transformants were obtained at rather a low frequency, with only twelve T1 lines being obtained from two selection attempts.

Physiology (Plate assays) Results. Five of eight 35S::GAL4-G682 lines were more tolerant than wild-type control seedlings in severe desiccation stress assays. Three of ten lines also performed marginally better than controls in a low N growth assay on the basis of having lower levels of anthocyanins.

Discussion. We have now isolated transformants that overexpress a version of the G682 protein that has a GAL4 activation domain fused to the N terminus. Transformants did not show a glabrous phenotype. Thus, with an added GAL4 domain, the G682 product no longer behaved as a repressor of trichome development.

In plate-based physiological assays, three often 35S::G682-GAL4 lines performed better than wild-type in a low-nitrogen root growth assay and five of ten were more tolerant in a dehydration assay. These results were less dramatic than those seen with 35S::G682.

Potential applications. 35S::G682-N-GAL4 lines may be used to confer drought-related tolerance or low nutrient tolerance to commercially-important plant species.

G682 (SEQ ID NO: 59 and 60; Arabidopsis thaliana)—Super Activation (C-GAL4-TA)

Background. The aim of this project was to determine whether the efficacy of the G682 protein could be improved by addition of an non-native GAL4 activation domain.

Morphological Observations. Overexpression of a “super-active” form of G682, comprising a GAL4 transactivation domain fused to the C terminus of the protein, produced a reduction in trichome density.

A total of seventeen 35S::G682-GAL4 T1 lines were obtained; one of these lines was completely glabrous, whereas the others were partially glabrous to varying extents. Three T2 lines were also examined and these also showed a partial glabrous phenotype.

Physiology (Plate assays) Results. 35S::G682-GAL4 lines were glabrous and had reduced anthocyanin levels. Five lines performed better in the C:N sensing and/or growth under low nitrogen assays.

Discussion. We have now isolated transformants that overexpress a version of the G682 protein that has a GAL4 activation domain fused to the C terminus. These lines all showed a reduction in trichome density, but the majority exhibited a partial, rather than a fully glabrous phenotype. Such effects were generally weaker than those shown by 35S::G682 transformants, where the majority of lines were completely glabrous. Thus, with an added GAL4 domain, the G682 product still behaved as a repressor of trichome development, but was less efficient than the wild-type version of the protein.

In plate-based physiological assays, a number of 35S::G682-GAL4 lines performed better than wild-type in low-nitrogen assays, but the results were less dramatic than those seen with 35S::G682. No clear-cut effects on root hair distribution were observed and no consistent effects were obtained in soil drought assays. These data indicate that the GAL4 domain reduces the activity of the G682 protein.

Potential applications. 35S::G682-C-GAL4 lines may be used to confer tolerance in low nutrient conditions to commercially-important plant species.

G682 (SEQ ID NO: 59 and 60; Arabidopsis thaliana)—RNAi (GS)

Background. The aim of this project was to farther refine our understanding of G682 function by use of an RNAi approach; a construct (P21111) was generated that was specifically targeted towards reducing G682 activity but not the activity of its paralogs (Table 18).

Morphological Observations. Sixteen T1 lines harboring the G682 RNAi (GS) construct P21111 were obtained. Three of these lines exhibited a moderate delay in the onset of flowering compared to wild-type controls (approximately 1 week late under 24 hour light). A fourth line showed a more subtle delay in the onset of flowering. The remaining twelve T1 plants appeared wild type at all developmental stages. Three T2 lines were also examined; plants from these populations showed no consistent differences in morphology to controls.

Physiology (Plate assays) Results. Eight of ten lines harboring a G682 RNAi (GS) construct were more tolerant than wild-type controls to sodium chloride in a germination assay. Five lines were more tolerant to sucrose than controls. Five lines were also less sensitive to ABA in a germination assay. Interesting, a similar insensitivity to ABA was noted in the KO.G682 lines and RNAi (clade) constructs. A total of three different lines were substantially more tolerant to heat than controls in germination or growth assays.

Discussion. We have isolated lines harboring a G682 specific RNAi construct. About 25% of T1 lines showed a mild delay in the onset of flowering, suggesting that the gene might promote the floral transition. However, a late-flowering phenotype was not evident in the T2 generation. With the exception of the late-flowering phenotype, these lines were wild-type.

Surprisingly, some of the lines showed more tolerance to NaCl and less sensitivity to ABA than controls in plate-based abiotic stress assays. The latter result is of interest since a G682 KO line also gave a positive result in ABA assays. In an initial run of a soil drought assay, though, G682 RNAi (GS) lines showed a wild-type performance.

Potential applications. The plate based results indicate that a knock-down approach with G682 may be used to afford stress tolerance. This is perhaps paradoxical, but it is possible that endogenous levels of G682 negatively regulate some genes which confer a benefit under stress conditions. An example of such an effect has recently been noted for a mutant of CBF2 (Novillo et al., 2004).

G682 (SEQ ID NO: 59 and 60; Arabidopsis thaliana)—RNAi (clade)

Background. The aim of this project was to further refine our understanding of G682 function by use of an RNAi approach; a construct (see sequence section) was generated that was targeted towards reducing activity of all members of the G682 clade. Given that the different members of the G682 clade potentially share some functional redundancy, it was thought that this method could reveal phenotypes that might not be visible in single KO lines for the individual clade members.

Morphological Observations. A total of twenty-three G682 RNAi (clade) lines were obtained. The majority of lines showed no consistent effects on morphology or development.

Four T1 lines were larger, developmentally more advanced, and showed upright leaves at the mid-rosette stage. Such phenotypes were not apparent in a second set of T1 lines. All plants from one T2 population and occasional plants from another T2 population were slightly larger than controls at the mid-rosette stage. The remainder of the T2 populations examined appeared wild type.

Physiology (Plate assays) Results. Five of ten lines harboring a G682 RNAi (clade) construct were tolerant to ABA in a germination assay. Some of these lines also were more tolerant in the cold growth (2/10 lines) and severe dehydration assays (2/10 lines) than wild-type controls.

Interestingly, a similar insensitivity to ABA was noted in the KO.G682 lines and RNAi (GS) construct lines.

Discussion. We have now isolated lines harboring the G682 RNAi clade construct. These lines displayed no clear differences in morphology to wild-type controls. In particular, no obvious changes in trichome morphology or distribution were observed. Given that null mutants for one of the clade members, G1816 (SEQ ID NO: 76), are known to exhibit alterations in trichome density, it would appear the construct used was not sufficient to completely eliminate activity of that gene. The lack of a trichome phenotype thus indicates that the lines do not have bona fide knockdown for all of the G682 clade members. Detailed expression studies would be needed to assess the effects on activity of the different G682-related Arabidopsis genes in these plants.

Surprisingly, five of ten lines were less sensitive to ABA in a plate assay; this result is of interest since a G682 KO line and the RNAi (GS) lines also gave a positive result in ABA assays. In an initial run of a soil drought assay, though, G682 RNAi (clade) lines showed a wild-type performance.

Potential applications. The plate based results indicate that a knock-down approach with G682 may be used to afford stress tolerance. This is perhaps paradoxical, but it is possible that endogenous levels of G682 negatively regulate some genes which confer a benefit under stress conditions. An example of such an effect has recently been noted for a mutant of CBF2 (Novillo et al., 2004).

G226 (SEQ ID NO: 61 and 62; Arabidopsis thaliana)—Constitutive 35S

Background. G226 (SEQ ID NO: 62) is a paralog of G682. In earlier studies, 35S::G226 lines showed enhanced resistance to osmotic stress conditions. The G226 sequence (GenBank accession AX651522) has been included in patent publication WO 03000898. Recently a genetic analysis of G226 was published, focusing on developmental phenotypes, in which the gene was identified as an enhancer of TRY and CPC (Kirik et al., 2004b).

The aim of this study was to re-assess 35S::G226 lines and determine whether overexpression of the gene could confer enhanced stress tolerance in a comparable manner to G682. We also sought to test whether use of a two-component overexpression system would produce any strengthening of the phenotype relative to the use of a 35S direct promoter-fusion.

Morphological Observations. We have now produced 35S::G226 lines using the two component system. These plants exhibited a comparable glabrous phenotype to the G226 overexpression lines using a 35S direct promoter fusion construct. Some lines developed slowly and flowered later than wild type. T1 lines generally were glabrous. Many of the lines were noted to show a distinct reduction in size compared to controls. A reduction in size was not previously noted for 35S::G226 direct promoter fusion lines, and could reflect the possibility that higher levels of G226 activity were obtained with the two-component system. Seven of 20 lines were severely dwarfed and died prior to reaching maturity.

Physiology (Plate assays) Results. All 35S::G226 lines were glabrous, had reduced anthocyanin levels and showed increased root hair production. Eight of nine 35S::G226 lines performed better in the C:N sensing and growth under low nitrogen assays.

Eight of ten lines were more tolerant to ABA in a germination assay. Five of ten lines were tolerant to sucrose. Two of these lines were tolerant to cold stress during germination and growth.

The observed tolerance to these abiotic stress could be related to the fact that 35S::G226 lines do not produce anthocyanins, or to the observation that the lines generally have enhanced root hair growth.

Discussion. We have generated multiple sets of 35S::G226 lines using the two component system. These lines showed a strong glabrous phenotype, similar to what was observed during our previous studies, and similar to the effect produced by G682 overexpression. In addition, many of the 35S::G226 lines were noted to be smaller than controls, an effect that had not been previously recognized. Many of the primary transformants died before reaching maturity. All 35S::G226 lines were glabrous, had reduced anthocyanin levels and showed increased root hair production.

35S::G226 lines have not yet been extensively tested in soil drought assays.

Potential applications. The current data support results of earlier studies indicating that G226 may be used to enhance abiotic stress tolerance. The positive results obtained in nitrogen assays indicate that G226 could be applied to enhance nutrient utilization traits. Based on the epidermal phenotypes shown by 35S::G226 lines, the gene might also be used to modify trichome or root hair development.

The reduction in size that was apparent in these lines suggests that G226 might require optimization by use of different promoters or protein modifications, prior to product development.

G226 (SEQ ID NO: 61 and 62; Arabidopsis thaliana)—Root ARSK1

Background. The aim of this project was to determine whether expression of G226 from an ARSK1 promoter, which drives expression in a root specific pattern, was sufficient to confer stress tolerance that is similar to, or better than, that seen in 35S::G226 lines.

Additionally this study allowed us to assess whether use of an alternative promoter could eliminate some of the undesirable size reductions that we have recently found to be associated with G226 overexpression, while still conferring enhanced stress tolerance.

Morphological Observations. ARSK1::G226 lines have been obtained using the 2-component system. The majority of these plants appeared wild type, but some size variation was apparent. Three T2 lines were later examined; plants from these populations were slightly small and slow developing relative to controls.

Physiology (Plate assays) Results. Three often ARSK1::G226 lines of seedlings were more tolerant than wild type in a cold growth assay.

Discussion. A total of thirty ARSK1::G226 lines have been obtained using the 2-component system. Most of the lines appeared wild type, but some were small and slow developing.

ARSK1::G226 plants showed no obvious increase in root hair density, as was seen in 35S::G226 lines. This latter result was perhaps unexpected given that ARSK1 drives expression within the root epidermis (see methods sections for details of promoter analysis). However, ARSK1 does not produce high level expression in the youngest differentiating region of the root; thus expression was likely not present in the regions where epidermal fate was being specified.

While ARSK1::G226 lines were more tolerant to cold than controls, expression of G226 from this root specific promoter was apparently not sufficient to produce a marked increase in tolerance to the other stresses tested. This could be because G226, like G682, has to be present in non-root tissues for stress tolerance to be attained, or because ARSK1 did not drive expression at high enough levels in the appropriate regions of the root.

Potential applications: Based on the results with overexpression lines, G226 is a candidate gene for increased cold stress tolerance.

G1816 (SEQ ID NO: 75 and 76; Arabidopsis thaliana)—Constitutive 35S

Background. G1816 corresponds to TRIPTYCHON (TRY), SEQ ID NO: 76, a gene that regulates epidermal cell specification in the leaf and root (Schnittger et al., 1998; 1999; Schellmann et al., 2002). G1816 is a paralog of G682 and was shown to confer increased resistance to osmotic stress conditions such as high levels of glucose.

The aim of this study was to re-assess 35S::G1816 lines and determine whether overexpression of the gene could confer enhanced stress tolerance in a comparable manner to G682. We also sought to examine whether use of a two-component overexpression system would produce any strengthening of the phenotype relative to the use of a 35S direct promoter-fusion.

Morphological Observations. 35S::G1816 lines produced using the two component system exhibited a comparable glabrous phenotype to G1816 overexpression lines produced using a 35S direct promoter fusion construct.

Three independent batches of 35S::G1816 two-component lines have been isolated. However, in addition to the glabrous effects, many of the lines were noted to be somewhat reduced in size compared to controls.

Line details are noted below:

Lines 301-315: Eight lines were completely glabrous. #303, 313 were partially glabrous. Of these lines, #301, 305, 311 were slightly smaller and slower developing than controls. #307, 308, 309, 314, 315 were very small and died early in the life cycle. No difference in coloration compared to wild-type was noted in the seed from this set of plants.

Lines 321-340: 14/20 lines were glabrous. 4/20 (#333, 334, 337, 339) were partially glabrous. All of the glabrous and partially glabrous plants were slightly reduced in size compared to wild type controls, at early stages of growth. 2/20 (#324, 332) appeared wild type. No difference in coloration compared to wild-type was noted in the seed from this set of plants.

Lines 341-354: all plants showed some evidence of a glabrous phenotype, with #346 being partially glabrous and the remainder being completely glabrous. All lines were smaller than wild type (20-70% wild-type size) at early stages of growth. No difference in coloration compared to wild-type was noted in the seed from this set of plants.

A number of T2 population were also morphologically examined. These plants showed a comparable phenotype to the primary transformants, being glabrous and generally smaller than controls.

Physiology (Plate assays) Results. 35S::G1816 lines were found to be insensitive to high glucose levels in a germination assay. 35S::G1816 leaves were glabrous and the plants also exhibited increased root hair density. We have now tested 35S::G1816 two-component lines; all ten of the lines tested showed excellent growth on sucrose in a germination assay. All these lines were also glabrous and displayed increased root hair density. These same lines performed well under our C:N sensing screen and in a root growth assays under low nitrogen, with 10 of 10 lines tested showing altered C/N sensing and better tolerance of low nitrogen conditions than controls.

Physiology (Soil Drought-Clay Pot) Summary. 35S::G1816 lines showed enhanced drought tolerance. Three independent (2-component) lines each showed significantly better survival than controls in a “whole pot” soil drought experiment.

TABLE 52
35S::G1816 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 304 | TCST | 2.0 | 0.89 | 0.062* | 0.38 | 0.15 | 0.00036* |
| 304 | TCST | 0 | 0 | 1.0 | 0.010 | 0.010 | 1.0 |
| 345 | TCST | 1.5 | 0.89 | 0.45 | 0.33 | 0.15 | 0.0034* |
| 345 | TCST | 0 | 0.17 | 1.0 | 0.042 | 0.042 | 1.0 |
| 353 | TCST | 2.0 | 0.89 | 0.028* | 0.58 | 0.15 | 0.0000000022* |
| 353 | TCST | 0 | 0.17 | 1.0 | 0.021 | 0.021 | 1.0 |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G1816 two component lines showed a strong glabrous phenotype, similar to what was observed during our previous studies, and similar to the effect produced by G682 overexpression. However, many of the 35S::G1816 lines were noted to be smaller than controls, an effect that had not been previously recognized.

These soil-drought results were generally comparable to those observed for G682 lines, indicating that the G682 and G1816 likely have very related functions.

Potential applications. Based on the tolerance of 35S::G1816 lines to osmotic stress, G1816 is a good candidate gene for use in the alleviation of drought related stress. The strong performance of 35S::G1816 lines on plates containing high levels of sugar particularly indicates that the gene might also be used to manipulate sugar-sensing responses. The strong performance in nitrogen assays indicates that this gene may be useful for engineering crops for growth in nutrient limited conditions. However, the decrease in size seen in some of the lines suggests that the gene might require optimization by use of different promoters or protein modifications prior to product development.

The epidermal phenotypes seen in 35S::G1816 lines indicate that the gene could also be used to modify developmental characters such as the formation of trichomes or root hairs.

G1816 (SEQ ID NO: 75 and 76; Arabidopsis thaliana)—Epidermal CUT1

Background. The aim of this project was to determine whether expression of G1816 from a CUT1 promoter, which predominantly drives expression the shoot epidermis, and results in high level expression in guard cells, is sufficient to confer stress tolerance. This study was also conducted to assess an alternative promoter for eliminating undesirable size reductions noted with constitutive overexpression, while still conferring enhanced stress tolerance.

Morphological Observations. Arabidopsis lines in which G1816 was expressed from the CUT1 promoter via the 2-component system exhibited no consistent differences in growth and development compared to controls. Two sets of CUT1::G1816 lines (461-470 and 481-500) have been obtained and all individuals exhibited wild-type morphology.

Physiology (Plate assays) Results. Six out of ten lines of CUT1::G1816 seedlings show less severe stress symptoms (i.e., had lower levels anthocyanins) than controls when grown on low nitrogen. Some of the lines also showed better root development compared to controls in these conditions. However, on normal control MS plates, the lines were not generally noted to have increase root hair density.

In the C:N sensing screen, CUT1::G1816 seedlings were wild-type in their response.

Discussion. CUT1::G1816 two component lines showed no consistent morphological differences to wild type and did not show a glabrous phenotype. This could indicate either that CUT1 did not produce high enough levels of G1816 activity in the epidermis to repress trichome initiation, or that activity of G1816 is required in sub-epidermal layers to cause that effect. A comparable result was observed in both CUT1::G682 and CUT1::G2718 lines.

CUT1::G 816 seedlings typically had less anthocyanins and in some cases better root development, relative to controls, on low nitrogen growth plates. No other significant results were obtained in plate assays, and no enhanced performance versus wild-type was seen in abiotic stress experiments.

Potential applications. The CUT1::G1816 combination was less effective for producing abiotic stress tolerance than constitutive expression. Nonetheless, this combination may be of utility for engineering tolerance to nutrient limited conditions, particularly since the CUT1::G1816 lines did not show any of the developmental off-types that were seen in 35S overexpression lines.

G1816 (SEQ ID NO: 75 and 76; Arabidopsis thaliana col)—KO

Background. The aim of this study is to determine whether G1816 is necessary as part of the plant's natural protection against drought-related stress, by obtaining and testing a null mutant under such conditions.

Morphological Observations. A G1816 T-DNA insertion line, SALK_{—}029760 (NCBI acc. no. BH789490, version BH789490.1; GI:19882588; SALK_{—}029760.51.00.x Arabidopsis thaliana TDNA insertion lines Arabidopsis thaliana genomic clone SALK_{—}029760.51.00.x, genomic survey sequence) was obtained from the ABRC at Ohio State University. BLAST analysis of the sequence from the insertion point deposited in GenBank by SALK indicated that the T-DNA in this line was integrated about 33 bp downstream of the G1816 start codon.

Two of twenty plants among individuals were observed to display irregularly spaced trichomes (lines 409, 410). The progeny of these lines were morphologically examined, and all showed irregularities in trichome spacing and structure. Trichomes appeared in unevenly spaced clusters, and in many cases exhibited four rather than three branches. Based on the trichome phenotype, which corresponds to the published try phenotype, and 100% KanR among those plants, it was concluded that the line 409 and 410 populations were homozygous.

Physiology (Plate assays) Results. G1816 knockout lines from two independent homozygous plants for the SALK insertion line: SALK_{—}029760 were more tolerant than wild type when germinated in the presence of 150 mM sodium chloride.

Discussion. We have identified a putative homozygous line for a T-DNA insertion within the G1816 sequence. These plants show a comparable phenotype to that described in the public literature for try mutants, and show irregularly spaced clusters of trichomes (Schnittger et al., 1998; 1999; Schellmann et al., 2002).

Potential applications. From our previous studies, we concluded that G1816 could be used to confer increased tolerance to a variety of abiotic stresses. The KO identified here may be useful in further elucidation of G682-related stress tolerance mechanisms. Additionally, the positive results seen in the plate based NaCl assay indicate that stress tolerance may be achieved through knock-down approaches on the G682 group.

G2718 (SEQ ID NO: 63 and 64; Arabidopsis thialiana)—Constitutive 35S

Background. G2718 (SEQ ID NO: 64) is a paralog of G682. The aim of this study was to re-assess 35S::G2718 lines and determine whether overexpression of the gene could confer enhanced stress tolerance in a comparable manner to G682.

Morphological Observations. 35S::G2718 lines were been obtained using the two-component system; we isolated twenty four lines (341-344; 421-440). These plants exhibited a comparable glabrous phenotype to the G2718 overexpression lines produced using a 35S direct promoter fusion construct.

It should be noted that many of the two-component lines showed a reduction in overall size, particularly at early stages of the life cycle. Such an effect was also noted for some of the lines obtained during our genomics program.

Three of four lines in the 341-344 set (all except #344) were completely glabrous. All of the lines in the 421-440 set were completely glabrous, except for #421, 422, 423, 424, 426, 430, 434, 436, 438, and 440, which exhibited a partially glabrous phenotype. Three lines were also examined in the T2 generation and each showed a glabrous phenotype combined with reduced size. No changes in seed coat coloration were noted in any of the lines.

Physiology (Plate assays) Results. Eight of ten 35S::G2718 lines were glabrous, and had reduced anthocyanin levels, showed increased root hair production. Eight of 10 lines were more tolerant than controls to growth assay in a sucrose germination assay. Nine of 10 lines exhibited altered C/N sensing relative to controls, and all ten lines tested were more tolerant than controls to low nitrogen conditions in a root growth determination.

Discussion. We have now isolated 35S::G2718 lines using the two component system. These lines showed a strong glabrous phenotype and increased root hair production, similar to what was observed during our initial genomics study, and similar to the effect produced by G682 overexpression. Many of the 35S::G2718 lines were noted to be smaller than controls. These lines also typically had reduced anthocyanin levels.

35S::G2718 lines typically were more tolerant than controls in sucrose germination assays and performed very well relative to controls in a low-nitrogen germination and growth assays (scoring higher than 35S::G682 lines in these assays). The observed tolerance to these abiotic stress could be related to the fact that 35S::G2718 lines do not produce anthocyanins, or to the observation that these lines generally have enhanced root hair growth.

Potential applications. The epidermal phenotypes seen in 35S::G2718 lines indicate that this gene could also be used to modify developmental characters such as the formation of trichomes or root hairs. The results from these experiments indicate that G2718 has a similar activity to G682 and could be used to enhance tolerance to abiotic stress and/or low nutrient conditions.

G3392 (SEQ ID NO: 71 and 72; Oryza sativa)—Constitutive 35S

Background. G3392 (SEQ ID NO: 71) is a rice ortholog of G682. The aim of this project was to determine whether G3392 has an equivalent function to the G682-related genes from Arabidopsis via the analysis of 35S::G3392Arabidopsis lines.

Morphological Observations. Overexpression of G3392 in Arabidopsis produced a glabrous phenotype and a slight reduction in overall plant size. Additionally, a loss of seed coat coloration was noted in some of the lines.

The above effects were observed in three different batches of 35S::G3392 lines as detailed below:

Lines 301-306: all plants were completely glabrous, and slightly small. 5/6 lines (all except #302) yielded pale seed; this effect was particularly strong in lines #301 and 305.

Lines 321-322: both plants were completely glabrous, slightly small and yielded pale seed.

Lines 341-349: all were completely glabrous and slightly small at all stages of growth. Seed from these lines were pale.

Three T2 populations were morphologically examined (see table below) and all showed equivalent phenotypes to those seen among T1 lines.

Physiology (Plate assays) Results. 35S::G3392 lines performed well in plate based assays in that they showed less stress symptoms than control plants. As indicated by anthocyanin production, all lines showed positive results in cold germination and cold growth assays. In addition, all lines gave positive results in a C:N sensing screen and in a root growth assay under low N. Better tolerance than controls was also noted for three lines (306, 342, and 346) in sucrose, mannitol, and NaCl germination assays.

Discussion. We have now generated 35S::G3392 lines; these plants showed comparable morphological effects to 35S::G682 lines and exhibited a glabrous phenotype combined with a reduction in overall size. Such similarities in phenotypes indicate that the proteins have similar activities. Interestingly, many of the 35S::G3392 lines also produced pale yellow seed, which likely indicated a reduction in anthocyanin levels in the seed coat. Such an effect was not observed 35S::G682 seed, but G682 and its paralogs were found during our genomics studies to inhibit anthocyanin production. It should be noted that the apparent tolerance of 35S::G3392 lines in many of the plate based assays might have been related to the absence of anthocyanins, rather than increased vigor per se.

Physiology (Soil Drought-Clay Pot) Summary. In clay-pot soil drought assays, results have been variable. Three 35S::G3392 lines were each assayed three separate times. One line was more tolerant to drought than controls.

Potential application: Based on the performance of 35S::G3392 in stress assays, the G3392 protein likely regulated some of the same pathways as G682. G3392 therefore has potential utility for stress resistance or nutrient utilization traits in commercial plants.

The effect of G3392 on epidermal patterning indicates that the gene could be applied to manipulate trichome development; in some species trichomes accumulate valuable secondary metabolites and in other instances are thought to provide protection against predation.

Change in seed coloration observed in 35S::G3392 lines indicates that the gene might also be used to regulate production of flavonoid related compounds, which affect the nutritional value of foodstuffs.

G3393 (SEQ ID NO: 65 and 66; Oryza sativa)—Constitutive 35S

Background. G3393 (SEQ ID NO: 65) is a closely-related rice homolog of G682. The aim of this project was to determine whether G3393 has an equivalent function to the G682-related genes from Arabidopsis via the analysis of 35S::G3393 Arabidopsis lines.

Morphological Observations. Overexpression of G3393 in Arabidopsis produced a glabrous phenotype and a slight reduction in overall plant size. Additionally, a loss of seed coat coloration was noted in some of the lines.

Line details, as compared to controls:

T1 lines 301-309: all plants were completely glabrous, and slightly small. A reduction in seed coat pigmentation was seen to various extents in the seed from these lines. Most of the lines showed a slight yellowing of the seed coat. Line 305, however, showed a strong effect and its seed were almost completely yellow. Seed from line 308 showed wild-type coloration.

T1 lines 321-333: all were slightly small and completely glabrous except for #329. #327 and 330 produced very pale seed. #328 and 332 produced slightly pale seed. #323, 326, 333 yielded very marginally lighter colored seed than wild type. Seed coloration in the remaining lines appeared wild type.

Three T2 populations were also examined; plants from all three of these populations were slightly small and glabrous. Interestingly, T2-323 plants and occasional plants from the other two T2 lines were early flowering. This phenotype was not noted on other plant dates or in the T1 generation, suggesting that it could have depended on environmental variables which might have differed between the plantings.

Physiology (Plate assays) Results. Nine of ten 35S::G3393 lines performed well in chilling growth assays as well as in the C:N sensing. All lines exhibited altered C/N sensing relative to controls. All lines also performed better than controls in a root growth assay in low nitrogen conditions. 35S::G3393 lines also showed a glabrous phenotype and exhibited increased root hair density relative to controls.

Discussion. We have now generated 35S::G3393 lines; these plants showed comparable morphological effects to 35S::G682 lines and exhibited a glabrous phenotype combined with a reduction in overall size. These similarities in phenotypes indicate that the proteins have similar activities. Interestingly, many of the 35S::G3393 lines also produced pale yellow seed, which likely indicated a reduction in anthocyanin levels in the seed coat. Such an effect was not observed 35S::G682 seed, but G682 and its paralogs were found during our genomics studies to inhibit anthocyanin production.

The better performance of 35S::G3393 lines in physiology assays may reflect reduced production of anthocyanins in 35S::G3393 seedlings.

In clay-pot soil drought assays, results have been variable.

Potential application: Based on the performance of 35S::G3393 in plate assays, it is clear that G3393 has potential utility for conferring abiotic stress resistance in commercial plants. However, the gene may need to be optimized for commercial application to mitigate the effects of G3393 on growth.

The effect of G3393 on epidermal patterning indicates that the gene could be applied to manipulate trichome development; in some species trichomes accumulate valuable secondary metabolites and in other instances are thought to provide protection against predation.

Change in seed coloration observed in 35S::G3393 lines indicates that the gene might also be used to regulate production of flavonoid related compounds, which affect the nutritional value of foodstuffs.

G3431 (SEQ ID NO: 67 and 68; Zea mays)—Constitutive 35S

Background. G3431 (SEQ ID NO: 67) is a closely-related maize homolog of G682. The aim of this project was to determine whether G3431 has an equivalent function to the G682-related genes from Arabidopsis via the analysis of 35S::G3431 Arabidopsis lines.

Morphological Observations. Overexpression of G3431 in Arabidopsis produced a glabrous phenotype and a slight reduction in overall plant size. Additionally, a loss of seed coat coloration was noted in some of the lines. These effects were observed in two different batches of 35S::G3431 lines as detailed below:

T1 lines 301-306: all plants were completely glabrous, and slightly small. A reduction in seed coat pigmentation was seen in lines #302, 303, 304, and 305. Lines 301 and 306 yielded wild-type colored seed.

T1 lines 321-340: all were slightly small and completely glabrous. Seeds from lines 335, 336, 337, 339 were pale in coloration. Other lines showed wild-type seed coloration.

Glabrous effects were also noted in each of three T2 populations. Plants from one of these populations, T2-303, also flowered early, but that effect was line specific and was not noted in the other lines.

Physiology (Plate assays) Results. Seven out often 35S::G3431 lines performed well in C:N sensing and growth under low nitrogen assays. The same lines also performed well in growth under chilling conditions. A small subset of these lines also did well in germination assays in the presence of high sucrose levels (versus controls). 35S::G3431 seedlings also showed a glabrous phenotype and three often lines had increased root hair density.

Discussion. We have now generated 35S::G3431 lines; these plants showed comparable morphological effects to 35S::G682 lines and exhibited a glabrous phenotype combined with a small reduction in overall size. These similarities in phenotypes indicate that the proteins have similar activities. Interestingly, some of the 35S::G3431 lines also produced pale yellow seed, which likely indicated a reduction in anthocyanin levels in the seed coat. Such an effect was not observed in 35S::G682 seed, but G682 and its paralogs were found during our genomics studies to inhibit anthocyanin production.

Surprisingly, in clay-pot soil drought assays, 35S::G3431 consistently showed greater sensitivity to drought.

We previously concluded that G3431 is equivalent to another maize gene, G3444 (SEQ ID NO: 69). However, it should be noted that the construct for G3431 gave a higher penetrance of positive results in the N assays, and the glabrous phenotype, compared to the G3444. This might be attributed to differences in the amounts of UTR included in the constructs.

Potential application: That 35S::G3431 gave an enhanced performance in some of the plate based assays indicates the gene may be used to effect abiotic stress tolerance. However, the gene would likely need to be optimized for commercial application to mitigate the effects of G3431 on growth.

The effect of G3431 on epidermal patterning indicates that the gene could be applied to manipulate trichome development; in some species trichomes accumulate valuable secondary metabolites and in other instances are thought to provide protection against predation.

Change in seed coloration observed in 35S::G3431 lines indicates that the gene might also be used to regulate production of flavonoid related compounds, which affect the nutritional value of foodstuffs.

G3444 (SEQ ID NO: 69 and 70; Zea mays)—Constitutive 35S

Background. G3444 is a maize gene closely related to G682. The aim of this project was to determine whether G3444 has an equivalent function to the G682-related genes from Arabidopsis via the analysis of 35S::G3444 Arabidopsis lines.

Morphological Observations. Overexpression of G3444 in Arabidopsis produced a glabrous phenotype and a reduction in overall plant size. Additionally, a loss of seed coat coloration was noted in some of the lines.

T1 Line Details:

Lines 321-340: 8/20 plants (#322, 323, 324, 325, 331, 333, 337, and 340) were glabrous and slightly small compared to wild type. The remaining lines showed no consistent differences in morphology to controls. Seed produced by lines #323, 324, 325 and 340 were slightly paler than wild type whereas seed from other lines showed wild-type coloration.

Three T2 populations were examined:

T2-331: plants were all slightly small and glabrous.

T2-333: plants were pale, rather early flowering, and had trichomes.

T2-334: plants were pale, rather early flowering, and had trichomes.

Physiology (Plate assays) Results. Four out of ten 35S::G3444 lines performed well in a root growth assay under low nitrogen. The plants also produced less anthocyanin and fewer trichomes. Three lines showed a higher density root hairs versus controls.

Physiology (Soil Drought-Clay Pot) Summary. One line was observed to recover from drought better than wild-type controls.

Discussion. We have now generated 35S::G3444 lines; these plants showed comparable morphological effects to 35S::G682 lines and exhibited a glabrous phenotype combined with a reduction in overall size. These similarities in phenotypes indicate that the proteins have similar activities. Interestingly, some of the 35S::G3444 lines also produced pale yellow seed, which likely indicated a reduction in anthocyanin levels in the seed coat. Such an effect was not observed 35S::G682 seed, but G682 and its paralogs were found during our genomics studies to inhibit anthocyanin production.

We previously concluded that G3444 is equivalent to another maize gene, G3431. However, it should be noted that the construct for G3431 gave a higher penetrance of positive results in the N assays, and the glabrous phenotype compared to the G3444. This might be attributed to differences in the amounts of UTR included in the constructs.

Potential applications. Based on the results obtained, G3444 could be used to effect abiotic stress tolerance (also see also G3431 results and discussion, above).

G3445 (SEQ ID NO: 83 and 84; Glycine max)—Constitutive 35S

Background. G3445 is a soy gene that is closely related to G682. The aim of this project was to determine whether G3445 has an equivalent function to the G682-related genes from Arabidopsis via the analysis of 35S::G3445 Arabidopsis lines.

Morphological Observations. Overexpression of G3445 in Arabidopsis produced a partial glabrous phenotype and a slight reduction in overall plant size. These effects were observed in three different batches of 35S::G3445 lines as detailed below:

Lines 301-303: line 301 was completely glabrous, lines 302 and 303 were partially glabrous. All lines three lines were slightly small and slower in developing and flowering than controls. Seed coloration was wild type in all three lines.

Lines 321-325: all were partially glabrous, but otherwise were wild type. Seed coloration was wild type in these lines.

Lines 341-347: all were glabrous to varying extents. Lines 341 and 344 showed the strongest effects and were also small versus wild type plants.

Three lines were morphologically examined in the T2 generation (301, 302, and 347); all exhibited a partial glabrous phenotype.

Physiology (Plate assays) Results. Four out of ten 35S::G3445 seedlings germinated in the presence of ABA. Some lines also produced fewer trichomes than control seedlings.

Discussion. 35S::G3445 lines exhibited a partially glabrous phenotype combined with a slight reduction in overall size, similar to 35S::G682. These phenotypic similarities indicate that the proteins have similar activities.

In plate-based physiological assays, we found that four of ten 35S::G3445 lines were ABA insensitive. There were no significant differences observed in the other plate-based physiology assays or in clay-pot soil drought assays.

Potential applications. Based on the results reported here, we have evidence that G3445 can be used to promote abiotic stress tolerance in commercial species.

The effect of G3445 on epidermal patterning indicates that the gene could be applied to manipulate trichome development; in some species trichomes accumulate valuable secondary metabolites and in other instances are thought to provide protection against predation.

G3446 (SEQ ID NO: 81 and 82; Glycine max)—Constitutive 35S

Background. G3446 (SEQ ID NO: 82) is a soy gene that is closely related to G682. The aim of this project was to determine whether G3446 has an equivalent function to the G682-related genes from Arabidopsis via the analysis of 35S::G3446 Arabidopsis lines.

Morphological Observations. Overexpression of G3446 in Arabidopsis produced a glabrous phenotype, a reduction in overall plant size, and alterations in flowering time.

Line Details:

T1 Lines 301-320: 18/20 were glabrous to various extents and also slightly small compared to controls. Of these lines, all were almost completely glabrous except for #302, 305, and 309 which were partially glabrous. 2/20 lines (#316, 320) appeared wild type. No obvious difference in seed coloration compared to controls was noted in this batch of lines.

Three lines were examined in the T2 generation (302, 303, and 311). All showed a glabrous phenotype. Alterations in flowering time were also seen, but these were rather inconsistent between lines. T2-302 plants were small and early flowering, whereas line 303 plants were late developing. Plants from line 311 flowered at the same time as controls.

Physiology (Soil Drought-Clay Pot) Summary. Three independent 35S::G3446 lines were tested in a single run of a whole pot soil drought assay. One of these lines (#302) showed significantly greater survival compared to controls. However, another line (#311) showed significantly worse survival. The third line (#303) showed a wild-type performance.

TABLE 53
35S::G3446 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 302 | DPF | 4.8 | 3.8 | 0.17 | 0.72 | 0.57 | 0.033* |

DPF = direct promoter fusion project
Survival = proportion of plants in each pot that survived
Drought scale 6 (highest score) = no stress symptoms, 0 (lowest score; most severe effect) = extreme stress symptoms
*line performed better than control (significant at P < 0.11)

Discussion. 35S::G3446 lines showed comparable morphological effects to 35S::G682 lines, as they exhibited a glabrous phenotype combined with a slight reduction in overall size. These similarities indicate that the proteins have similar activities.

We previously concluded that G3446 is equivalent to another soy gene, G3447. The phenotypes of 35S::G3446 and 35S::G3447 were equivalent. Both genes were subjected to plate-based assays as well as clay-pot soil drought assays. 35S::G3446 and 35S::G3447 both showed no consistent differences to wild-type in plate-based physiological assays, and showed no clear-cut effects on root hair density. They also both showed equivocal results in clay-pot soil drought assays. Although some lines showed an increased tolerance to drought (e.g., line 302), other lines showed a worse performance compared to wild-type.

These results demonstrate that within the G682 study group, the glabrous phenotype is separable from both the effects on root hair patterning and the stress tolerance phenotypes.

Potential applications. Given the inconsistent soil-drought data, and the wild-type performance in plate assays, it remains to be determined whether G3446 could be applied to effect abiotic stress tolerance in commercial species. However, based on the soil assay results, there is some indication that under certain conditions, G3446/G3447 may enhance drought tolerance.

The effect of G3446 on epidermal patterning indicates that the gene could be applied to manipulate trichome development; in some species trichomes accumulate valuable secondary metabolites and in other instances are thought to provide protection against predation.

G3447 (SEQ ID NO: 85 and 86; Glycine max)—Constitutive 35S

Background. G3447 is a soy gene that is closely related to G682. The aim of this project was to determine whether G3447 has an equivalent function to the G682-related genes from Arabidopsis via the analysis of 35S::G3447 Arabidopsis lines.

Morphological Observations. Overexpression of G3447 in Arabidopsis produced a glabrous phenotype and a reduction in overall plant size.

All lines from batch 301-320 were either completely glabrous or almost completely glabrous. Additionally many of the plants displayed a slight reduction in overall size compared to controls. No obvious difference in seed coloration compared to controls was noted in this batch of lines.

Three T2 lines were later examined. Plants from each of these populations were glabrous and showed small rosettes. Plants from one of the lines (#316) were also early flowering. This effect was not seen in the other lines and had not been noted in the T1 generation.

Physiology (Plate assays) Results. Three lines of 35S::G3447 seedlings produced less anthocyanin in a root growth assay under low nitrogen. 35S::G3447 seedlings did not produce trichomes.

Physiology (Soil Drought-Clay Pot) Summary:

Seven independent 35S::G3447 lines were examined in soil drought assays. One of these lines (#310) exhibited a significantly better survival and recovery than wild-type controls on two different plant dates.

TABLE 54
35S::G3447 drought assay results:

| | | | Mean | | | | |
|---|---|---|---|---|---|---|---|
| | | Mean | drought | p-value for | Mean | Mean | |
| | Project | drought | score | drought score | survival for | survival for | p-value for difference |
| Line | Type | score line | control | difference | line | control | in survival |
| 310 | DPF | 3.8 | 3.8 | 0.83 | 0.54 | 0.57 | 0.65 |
| 310 | DPF | 1.5 | 0.60 | 0.016* | 0.14 | 0.050 | 0.017* |
| 310 | DPF | 3.6 | 1.3 | 0.0033* | 0.67 | 0.25 | 0.0000000000085* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3447 lines showed comparable morphological effects to 35S::G682 lines, as they exhibited a glabrous phenotype combined with a slight reduction in overall size. These similarities indicate that the proteins have similar activities.

We previously concluded that G3447 is equivalent to another soy gene, G3446. The phenotypes of 35S::G3446 and 35S::G3447 were similar. Lines for both genes were subjected to plate-based assays as well as clay-pot soil drought assays, and similar results were obtained. 35S::G3446 and 35S::G3447 both showed no clear-cut difference to wild-type in plate-based physiological assays. However, a small number of the G3447 lines were more tolerant than wild-type controls in low N growth assays. Results for 35S::G3447 lines from the clay-pot soil drought assays were also somewhat inconclusive. A single line showed enhanced tolerance (line 310, in triplicate assays planted on three different dates), but other lines showed a worse performance than controls in one or more plantings.

These results demonstrate that within the G682 study group, the glabrous phenotype is separable from both the effects on root hair patterning and the stress tolerance phenotypes.

Potential applications. Given the soil-drought data, it is possible that under some conditions that G3446/G3447 may promote tolerance to drought-related stress. However, the gene appears to be less effective in this regard than G682.

The effect of G3447 on epidermal patterning indicates that the gene could be applied to manipulate trichome development; in some species trichomes accumulate valuable secondary metabolites and in other instances are thought to provide protection against predation.

G3448 (SEQ ID NO: 79 and 80; Glycine max)—Constitutive 35S

Background. G3448 (SEQ ID NO: 79) is a soy gene that is closely related to G682. The aim of this project was to determine whether G3448 has an equivalent function to the G682-related genes from Arabidopsis via the analysis of 35S::G3448 Arabidopsis lines.

Morphological Observations. Overexpression of G3448 in Arabidopsis produced a glabrous phenotype and a reduction in overall plant size.

All twenty T1 lines from batch 301-320 were either completely glabrous or almost completely glabrous. Additionally, many of the plants displayed a reduction in overall size compared to controls. The most strongly affected lines (#311, 313, 315, 320) were particularly small and had leaf tissue that was paler than that of wild type. A number of T2 populations (see table below) from these line were examined and comparable phenotypes were seen to those in the T1 plants.

No obvious difference in seed coloration compared to controls was noted in 35S::G3448 lines.

Physiology (Plate assays) Results. All ten 35S::G3448 lines performed better in the C:N sensing and growth under low nitrogen assays. All lines produced more root hairs, and lacked trichomes. Three of ten lines also were more tolerant than wild-type in a chilling growth assay.

Physiology (Soil Drought-Clay Pot) Summary. One line of 35S::G3448 plants (# 308) exhibited significantly better survival than controls.

TABLE 55
35S::G3448 drought assay results

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 308 | DPF | 4.0 | 2.8 | 0.21 | 0.58 | 0.41 | 0.0041* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3448 showed phenotypes that were similar to 35S::G682 lines, exhibiting a glabrous phenotype combined with a reduction in overall size. These similarities in phenotypes indicate that the proteins have similar activities. Additionally the 35S::G3448 lines showed a somewhat lighter coloration than controls, perhaps indicating that levels of pigments such as anthocyanins were reduced in leaf tissue.

In plate-based physiological assays three of ten 35S::G3448 lines were tolerant of chilling, and all lines tested did well in low-nitrogen root growth and C/N sensing assays. The lines also showed increased root hair density. The better performance than controls might reflect the plants inability to make anthocyanins.

In clay-pot soil drought assays, a single line (#308) showed a drought resistant phenotype.

Potential applications. Based on the physiology results obtained, G3448 may be applied to effect abiotic stress tolerance in commercial species.

The effect of G3448 on epidermal patterning indicates that the gene could be applied to manipulate trichome development; in some species trichomes accumulate valuable secondary metabolites and in other instances are thought to provide protection against predation. The lighter coloration of 35S::G3448 plants could indicate that G3448 may be used to regulate the production of flavonoid related compounds, which contribute to the nutritional value of foodstuffs.

G3449 (SEQ ID NO: 77 and 78; Glycine max)—Constitutive 35S

Background. G3449 (SEQ ID NO: 77) is a soy gene that is a closely-related homolog of G682. The aim of this project was to determine whether G3449 has an equivalent function to the G682-related genes from Arabidopsis via the analysis of 35S::G3449 Arabidopsis lines.

Morphological Observations. Overexpression of G3449 in Arabidopsis produced a glabrous phenotype and a reduction in overall plant size.

Eighteen of twenty lines from batch 301-320 were either completely glabrous or almost completely glabrous. Two of twenty lines (#302, 304) appeared wild type. All of the glabrous plants were markedly small and in most instances developed more slowly than wild-type controls. These lines also were somewhat paler than controls at the seedling stage. No obvious alteration in coloration was observed in the seed from this batch of plants.

Three T2 populations were also examined; the plants were small and showed glabrous effects, as had been seen in the T1.

Physiology (Plate assays) Results. Nine out of ten 35S::G3448 lines performed better in the C:N sensing and growth under low nitrogen assays. Some lines produced less anthocyanin in cold conditions than wild type. All lines produced more root hairs, and showed a glabrous phenotype.

Discussion. We have now generated 35S::G3449 lines; these plants showed comparable morphological effects to 35S::G682 lines and exhibited a glabrous phenotype combined with a slight reduction in overall size. These similarities in phenotypes indicate that the proteins have similar activities. Additionally, 35S::G3449 transformants were distinctly paler than wild-type at the seedling stage, perhaps indicating a reduction in the levels of pigments such as anthocyanins. The better performance in C/N sensing and cold assays may reflect the plants reduced production of anthocyanins.

Potential applications. Based on the results of plate assays, G3449 may be applied to effect abiotic stress tolerance. However, the gene appears to be less effective for drought related stress than G682.

The effect of G3449 on epidermal patterning indicates that the gene could be applied to manipulate trichome development; in some species trichomes accumulate valuable secondary metabolites and in other instances are thought to provide protection against predation. The lighter coloration of 35S::G3449 plants could indicate that G3449 may be used to regulate the production of flavonoid related compounds, which contribute to the nutritional value of foodstuffs.

G3450 (SEQ ID NO: 73 and 74; Glycine max)—Constitutive 35S

Background. G3450 (SEQ ID NO: 73) is a soy gene that is a closely-related homolog of G682. Based on a phylogenetic tree built using conserved MYB domains, the G3450 protein appears to be more closely related to the G682-clade of Arabidopsis genes than any of the other homologs included the study. The aim of this project was to determine whether G3450 has an equivalent function to the G682-related genes from Arabidopsis via the analysis of 35S::G3450 Arabidopsis lines.

Morphological Observations. Overexpression of G3450 in Arabidopsis produced a glabrous phenotype and a slight reduction in overall plant size. Additionally, a loss of seed coat coloration was noted in some of the lines. These effects were observed in three different batches of 35S::G3450 T1 lines as detailed below:

Lines 301-318: all plants were completely glabrous or almost completely glabrous, slightly small, and rather pale in coloration compared to controls. Three of the eighteen lines (#311, 312, and 314) were very small and perished prior to flowering. The seed coloration from this batch of lines was generally marginally paler than in the controls. The strongest reduction in seed coloration was seen in line #309 and the seed from that line were also slightly larger than in wild type.

Lines 321-336: all plants were completely glabrous or almost completely glabrous, and slightly small. No obvious alterations in seed coloration were observed in this batch of lines.

Lines 341-360: all plants were completely glabrous and slightly small. 2/20 lines showed accelerated flowering.

Three lines T2-304, T2-315 and T2-317 were later examined in the T2 generation. All plants from those populations were glabrous, slightly small, and slightly late developing compared to wild type.

Of the lines submitted for physiological testing, the following showed a segregation on selection plates in the T2 generation that was compatible with the transgene being present at a single locus: 304, 305, 307, 313, 315, and 317. Lines 301, 302, 303 showed segregation that was compatible with insertions at multiple loci.

Physiology (Plate assays) Results. All 35S::G3450 lines were glabrous, had reduced anthocyanin levels and showed increased root hair production. Nine of ten 35S::G3450 lines performed better in the C:N sensing and growth under low nitrogen assays.

Six often lines (#302, 303, 304, 307, 315, and 317) were more tolerant of cold stresses on germination. Two of these lines (315, 317) showed an enhanced performance in salt germination assays and one of the lines (302) showed increased heat tolerance on germination. Enhanced tolerance was also observed in growth assays under heat stress (304, 307, and 315) and chilling (303, 307, 313, 315, and 317). Two often lines (306, 315) also showed an enhanced performance in a severe dehydration assay.

Physiology (Soil Drought-Clay Pot) Summary. Overexpression of G3450 produced a marked increase in drought tolerance in Arabidopsis. Four independent lines were tested and three of these lines showed positive effects.

Line 317 showed significantly better survival than wild type in 2 of 3 plantings. Line 315 showed significantly better survival than wild type in 1 of 3 plantings. Line 304 showed significantly better survival than wild type in 1 of 1 planting.

TABLE 56
35S::G3450 drought assay results:

| | | | Mean | | | | |
|---|---|---|---|---|---|---|---|
| | | Mean | drought | p-value for | Mean | Mean | |
| | Project | drought | score | drought score | survival | survival for | p-value for difference in |
| Line | Type | score line | control | difference | for line | control | survival |
| 304 | DPF | 2.6 | 0.60 | 0.0024* | 0.67 | 0.13 | 0.0000000000000025* |
| 315 | DPF | 2.7 | 2.8 | 0.85 | 0.38 | 0.41 | 0.62 |
| 315 | DPF | 1.9 | 1.5 | 0.43 | 0.25 | 0.32 | 0.38 |
| 315 | DPF | 2.3 | 0.10 | 0.00016* | 0.66 | 0 | 0.0000016* |
| 317 | DPF | 2.3 | 2.8 | 0.64 | 0.38 | 0.41 | 0.56 |
| 317 | DPF | 3.8 | 1.8 | 0.0065* | 0.67 | 0.35 | 0.00000024* |
| 317 | DPF | 2.1 | 0.10 | 0.00012* | 0.55 | 0.0071 | 0.00000061* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have now generated 35S::G3450 lines; these plants showed comparable morphological effects to 35S::G682 lines and exhibited a glabrous phenotype combined with a slight reduction in overall size. These similarities in phenotypes indicate that the proteins have similar activities. Interestingly, 35S::G3450 lines were slightly pale and some of the lines produced pale yellow seed, which likely indicated a reduction in anthocyanin levels in the seed coat. Such an effect was not observed in 35S::G682 seed, but G682 and its paralogs were found during our genomics studies to inhibit anthocyanin production. The observed tolerance to some of these abiotic stress could be related to the fact that 35S::G3450 lines do not produce anthocyanins, or to the observation that these lines generally have enhanced root hair growth.

The comparable morphological and physiological effects obtained in 35S::G3450 lines versus overexpression lines for the G682-related Arabidopsis genes, indicates that the G3450 protein has a very similar or equivalent activity to the Arabidopsis proteins.

Potential applications. Based on the positive results of plate based assays and soil drought assays, G3450 could be used to confer tolerance to drought-related and low nutrient stress.

The effect of G3450 on epidermal patterning also indicate that the gene could be applied to manipulate trichome development; in some species trichomes accumulate valuable secondary metabolites and in other instances are thought to provide protection against predation. The lighter coloration observed in 35S::G3450 leaf tissue and seeds could indicate that G3450 may be used to regulate the production of flavonoid related compounds, which contribute to the nutritional value of foodstuffs.

### The G867 Clade

G867 (SEQ ID NO: 87 and 88; Arabidopsis thaliana)—Constitutive 35S

Background. G867 corresponds to RAV1 (Kagaya et al., 1999) and was selected for the drought program based on the enhanced resistance of 35S::G867 lines to abiotic stress treatments. G867 has been reported as involved in a brassinosteroid signaling pathway, and was found to cause reductions in lateral root and rosette leaf development when overexpressed, and reduction in G867 gene expression causes early flowering (Hu et al., 2004).

Previously, we observed that G867 overexpression produced enhanced tolerance to high salt and sucrose levels. The aim of this study was to re-assess 35S::G867 lines and compare its overexpression effects to those of its paralogs. We also sought to test whether use of a two-component overexpression system would produce any strengthening of the phenotype relative to the use of a 35S direct promoter-fusion.

Morphological Observations. Direct promoter fusion lines: Additional G867 overexpression lines have now been generated containing the 35S direct promoter fusion construct (P383). 35S::G867 plants displayed a number of pleiotropic and variable alterations in overall morphology relative to wild-type controls. Such developmental changes included a reduction in overall size and alterations in leaf orientation. In some lines, changes in leaf shape, flowering time and non-specific floral abnormalities that reduced fertility were observed. A number of the lines (#5, 6, 8), were also re-examined, and were found to display similar phenotypes to that observed previously. Details of lines are shown below:

T1 lines 301-320: all lines appeared slightly reduced in size and #304, 310, 316, 318 showed abnormalities in rosette leaf orientation.

T2-305: all were rather small, marginally early flowering, had rather narrow leaves, and show floral abnormalities (flowers fail to properly open).

T2-306: all were rather small, marginally early flowering, had rather narrow leaves, and show floral abnormalities (flowers fail to properly open).

T2-309: all were small, had narrow, upward oriented leaves, and showed abnormal flowers.

T2-310: 3/6 wild-type, 1/6 dwarfed and infertile, 2/6 early flowering but otherwise wild type.

T2-312; all appeared wild type.

T3-5: all plants were distinctly smaller than controls, slightly pale and have narrow leaves.

T3-6: all plants were distinctly smaller than controls, and 4/7 were tiny, dark in color, and perished early in development.

T3-8: 2/7 plants examined were tiny and died at early stages of development, 5/7 were distinctly small.

2-Component Lines

Further G867 overexpression lines (1621-1640) were produced using the two component vector system (P7140, P6506). These plants showed comparable phenotypes to those seen in the direct fusion lines and were small, slow developing and showed alterations in leaf shape and orientation.

T1 lines 1621-1640: all are small, pale, and slow developing to various extents. Some lines show alterations in leaf orientation.

T2-1622: all slightly small.

T2-1626: all slightly small and were slow developing at early stages.

T2-1633: 3/6 slightly small and slow developing. Others appeared wild type.

Physiology (Plate assays) Results. 35S::G867 lines had previously been shown to exhibit increased seedling vigor in germination assays on both high salt and high sucrose media compared to wild-type controls. We confirmed these data using both direct promoter fusion and two component systems and extended the positive results to include insensitivity to ABA in a germination assay (as opposed to ABA sensitive wild-type plants). Furthermore, several lines had better growth than wild-type plants in a chilling growth assay. However, several lines had small and chlorotic seedlings and showed a low germination efficiency. A number of lines also exhibited increased root hair density.

Discussion. We have now examined an additional set of 35S::G867 direct promoter-fusion lines. Overall, 35S::G867 causes a number of morphological phenotypes, including a reduction in overall size, alterations in leaf shape and orientation (which potentially indicated a disruption in circadian control), slow growth, and floral abnormalities relative to controls. Both direct fusion and two-component lines have been generated and assayed for drought related stress tolerance.

It should be emphasized that we have obtained comparable developmental effects as well as a strong enhancement of drought related stress tolerance in overexpression lines for the all three of the paralogs; G9, G1930 and G993. The almost identical phenotypic effects observed for the four genes strongly indicate that they are functionally equivalent.

Potential applications. Based on the results of our overexpression studies, G867 and its related homologs are excellent candidate genes for improvement of drought related stress tolerance in commercial species. However, the morphological effects associated with their overexpression, suggests that tissue-specific or conditional promoters might be required to optimize the utility of these genes.

G867 (SEQ ID NO: 87 and 88; Arabidopsis thaliana)—Vascular SUC2

Background. The aim of this project is to determine whether expression of G867 from a SUC2 promoter, which predominantly drives expression in a vascular specific pattern, is sufficient to confer stress tolerance that is similar to, or better than, that seen in 35S::G867 lines. We also wish to assess whether use of the SUC2 promoter could eliminate some of the undesirable morphologies associated with G867 overexpression, while still conferring enhanced stress tolerance.

Morphological Observations. Two-component lines have been obtained (#381-400) for which an opLexA::G867 construct was supertransformed into a SUC2::LexA-GAL4TA promoter driver line (#6). These lines appeared wild type at all developmental stages. However, it should be noted that the promoter driver line (#6) used in this set of lines, produced relatively low expression levels.

A direct promoter-fusion construct (P21521) for SUC2::G867 was also available. Fourteen lines (#1581-1594) harboring this construct also showed no consistent differences to wild-type controls.

A number of populations from both the 2-component (3 lines) and direct fusion (6 lines) were examined in the T2 generation, as indicated in the table below. These plants showed no consistent differences to wild-type, except for plants from the T2-1583 population, some of which were found to be small and exhibit early senescence. This phenotype was not recorded in other lines or in T2-1583 plants grown for the soil drought assay.

Physiology (Plate assays) Results. Seven out of ten SUC2::G867 (2-component) lines were more tolerant to sodium chloride in a germination assay. Four of these seven SUC2::G867 lines also performed better in a sucrose germination assay.

Positive results were also obtained with SUC2::G867 direct fusion lines. Three of ten direct fusion SUC2::G867 lines performed well in the sucrose germination assay. Six of ten direct fusion SUC2::G867 lines were more tolerant to ABA than wild type. In addition, five of these six direct fusion lines perform better than wild type in response to severe dehydration.

Physiology (Soil Drought—Clay Pot) Summary. Overall, SUC2::G867 lines showed an enhanced performance in soil drought assays.

Three 2-component lines were tested in a single run of a “split pot” assay (line and control plants together in same pot). Two of the three lines (#385, 388) exhibited significantly better survival than controls.

Six different SUC2::G867 direct fusion lines were also tested in “whole pot” soil drought assays. Two of these lines (1592 and 1583) each showed better survival than wild type on a single plant date.

TABLE 57
SUC2::G867 drought assay results:

| | | | | | p-value for | | | |
|---|---|---|---|---|---|---|---|---|
| | | | Mean | Mean | drought | Mean | Mean | p-value for |
| | Project | | drought | drought | score | survival | survival for | difference in |
| Line | Type | Assay type | score line | score control | difference | for line | control | survival |
| 1583 | DPF | Whole pot | 2.3 | 0.70 | 0.0051* | 0.34 | 0.14 | 0.00012* |
| 1583 | DPF | Whole pot | 2.3 | 2.1 | 0.60 | 0.42 | 0.48 | 0.34 |
| 1592 | DPF | Whole pot | 1.7 | 1.2 | 0.17 | 0.22 | 0.22 | 0.91 |
| 1592 | DPF | Whole pot | 2.1 | 1.0 | 0.12 | 0.43 | 0.28 | 0.010* |
| 385 | TCST | Split pot | 1.3 | 0.75 | 0.095* | 0.19 | 0.11 | 0.059* |
| 388 | TCST | Split pot | 1.5 | 0.33 | 0.041* | 0.21 | 0.048 | 0.041* |

DPF = direct promoter fusion project
TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have isolated SUC2::G867 lines via both a direct-promoter fusion approach and a 2-component approach.

The physiological effects of SUC2::G867 in stress assays were comparable, but perhaps slightly weaker, than those shown by 35S::G867 plants.

Importantly, it should be noted that in contrast to the phenotype seen in 35S::G867 transformants, which showed a variety of undesirable morphological effects, SUC2::G867 lines displayed no obvious developmental abnormalities. Thus, the SUC2 promoter rather than the CaMV 35S promoter might alleviate such problems.

Potential applications. Given the undesirable morphologies that arise from G867 overexpression, it might be helpful to optimize G867 expression in plants by use of alternative promoters or sequence modifications. The results of this experiment indicate that use of the SUC2 promoter may be a good means to achieve this.

G867 (SEQ ID NO: 87 and 88; Arabidopsis thaliana)—Root ARSK1

Background. The aim of this project is to determine whether expression of G867 from an ARSK1 promoter, which drives expression in a root specific pattern, is sufficient to confer stress tolerance that is similar to, or better than, that seen in 35S::G867 lines. We also wish to assess whether use of a root specific promoter can eliminate some of the undesirable morphologies associated with G867 overexpression, while still conferring enhanced stress tolerance.

Morphological Observations. ARSK1::G867 lines exhibited no consistent changes in morphology versus wild-type controls.

Two Batches of 2-Component Lines Obtained:

T1 lines 1681-1689; at early stages, most of these plants were distinctly smaller than controls. However, at later stages, they appeared wild type.

T1 lines 1741-1748; some size variation was noted at early stages, but otherwise the plants appeared wild type.

T2 lines 1741, 1744 and 1748; wild-type at all developmental stages.

Direct promoter-fusion lines: To confirm the effects of ARSK1 with G867, we also obtained lines harboring a direct promoter-fusion construct. Twenty lines were obtained (1901-1920) and these plants showed no consistent differences to wild-type controls.

Physiology (Plate assays) Results. Three ARSK1::G867 lines were insensitive to ABA in a germination assay.

Physiology (Soil Drought-Clay Pot) Summary. Data from assays run so far indicate that the ARSK1::G867 combination affords significant drought tolerance relative to controls. Three independent lines were tested on each of three different plant dates. Lines 1744 and 1748 both showed a better performance than controls on two of the dates and a comparable performance to controls on the third date. Line 1741 showed a better performance than controls on one date and a wild-type performance on two other dates

TABLE 58
ARSK1::G867 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 1741 | TCST | 1.8 | 0.33 | 0.017* | 0.26 | 0.056 | 0.000098* |
| 1741 | TCST | 0.90 | 0.50 | 0.22 | 0.26 | 0.26 | 1.0 |
| 1741 | TCST | 1.2 | 0.80 | 0.12 | 0.14 | 0.17 | 0.51 |
| 1744 | TCST | 1.3 | 0.33 | 0.095* | 0.26 | 0.056 | 0.000098* |
| 1744 | TCST | 2.1 | 1.8 | 0.45 | 0.50 | 0.50 | 0.95 |
| 1744 | TCST | 1.8 | 1.1 | 0.024* | 0.38 | 0.34 | 0.45 |
| 1748 | TCST | 1.3 | 0.33 | 0.032* | 0.26 | 0.056 | 0.00060* |
| 1748 | TCST | 1.9 | 1.2 | 0.024* | 0.45 | 0.37 | 0.16 |
| 1748 | TCST | 1.2 | 1.0 | 0.52 | 0.24 | 0.24 | 0.92 |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have generated a number of ARSK1::G867 lines using a two-component approach, and these lines appeared to have wild-type morphology. Wild-type morphology was also seen in ARSK1::G867 direct fusion lines. These results contrast the effects of 35S overexpression of G867, which produces a marked reduction in overall size and other developmental abnormalities (see 35S::G867 report). It appears that targeting G867 expression to the roots can retain a number of desirable drought-tolerance related phenotypes, while eliminating the undesirable shoot morphology apparent in the 35S::G867 lines.

Potential applications. Based on the data from overexpression studies, G867 is a good candidate gene for improving stress tolerance in commercial species. However, given the undesirable morphologies that arise from G867 overexpression, it may be helpful to optimize the gene by use of alternative promoters or sequence modifications before it can be used to develop products. The results of this experiment indicate that use of the ARSK1 promoter may be a good way to achieve this.

G867 (SEQ ID NO: 87 and 88; Arabidopsis thaliana)—Stress Inducible RD29A—Line 2

Background. A two component approach was used for these studies and two different RD29A::LexA promoter driver lines were established: line 2 and line 5. Line 2 had a higher level of background expression than line 5, and thereby is expected to provide somewhat different regulation. Line 2 was observed to have constitutive basal expression of GFP, and to have a marked increase in GFP expression following the onset of stress. In contrast, line 5 exhibited very low background expression, although it still exhibited an up-regulation of expression following the onset of stress. However, the stress-induced levels of GFP expression observed in line 5 were lower than those observed for line 2.

Morphological Observations. Two batches of supertransformants for opLexA::G867 into the RD29A_line2::LexA promoter background were obtained (1381-1398; 1561-1567). Most of the primary transformants were smaller than controls, to varying extents, but in other respects, showed wild-type morphology.

Line Details:

Lines 1381-1398: all lines were slightly small except for 6/19 lines (1383, 1389, 1391, 1392, 1384, 1393) which were tiny.

Four T2 populations from this set of lines were later examined (see table below). Plants from each of these T2 showed no consistent differences in morphology to wild-type controls.

Lines 1561-1567: at early stages, all appeared wild type, but at late stages all were noticeably smaller than wild type.

Physiology (Plate assays) Results. Four RD29A::G867 lines (2-component in the line 2 promoter background) out of ten performed well in a germination assay in the presence of sodium chloride.

Physiology (Soil Drought-Clay Pot) Summary. Four independent G867 (2-component) lines in the RD29A line 2 promoter background were each tested in a single run of the split pot soil drought assay. One of these lines (#1391) showed a significantly better performance than controls.

TABLE 59
RD29A::G867 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 1391 | TCST | 0.83 | 0.25 | 0.042* | 0.26 | 0.12 | 0.0092* |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. The majority of the RD29A::LexA;opLexA::G867 lines were slightly smaller than controls, but in other respects exhibited wild-type morphology. Thus, the low constitutive expression produced by the driver line could have triggered such reduced size effects. The reduction in size seen in these lines was generally less severe than that seen in the 35S::G867 lines.

Potential applications. G867 in combination with a stress inducible promoter appears to confer moderate drought tolerance and only moderate morphological defects (small size) compared to the 35S::G867 lines, indicating that this may be a potential way to achieve stress tolerance while minimizing undesirable morphologies.

G867 (SEQ ID NO: 87 and 88; Arabidopsis thaliana)—Stress Inducible RD29A—Line 5

Morphological Observations. Supertransformants for opLexA::G867 into the RD29A_line5::LexA promoter background showed no consistent differences in morphology to controls.

A total of thirty-five lines have been obtained, in two different batches: (#1401-1418 and 1461-1477). Two lines were very small (#1402 and 1464), but the remainder were wild type. Plants from two T2 populations were also examined and appeared wild type.

Physiology (Plate assays) Results. Three of ten RD29A::G867 lines performed better than wild-type seedlings in a germination assay in the presence of sodium chloride (one line performed substantially better than controls).

Physiology (Soil Drought-Clay Pot) Summary. One line (line 1466) comprising an opLexA::G867 transgene transformed into the RD29A line 5 promoter background recovered from drought better than controls in soil-based assays.) on one plant date.

TABLE 60
RD29A::G867 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 1466 | TCST | 0.60 | 0.10 | 0.28 | 0.20 | 0.19 | 0.88 |
| 1466 | TCST | 0.60 | 0.10 | 0.28 | 0.11 | 0.029 | 0.0098* |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have now established two component (RD29A::LexA;opLexA::G867) lines in the RD29A line 5 background. These lines showed no consistent differences in morphology to controls. This result contrasts the effects of 35S overexpression of G867, which produces a marked reduction in overall size and other developmental abnormalities (i.e., as compared to 35S::G867 lines).

Potential applications. This experiment indicates that G867 in combination with a stress inducible promoter may effect drought-related abiotic stress tolerance, while reducing undesirable morphological effects associated with constitutive overexpression of the gene. However, it should be noted that the stress tolerance phenotypes obtained with RD29A were less compelling than those obtained with the 35S::G867 lines.

G867 (SEQ ID NO: 87 and 88; Arabidopsis thaliana col)—Leaf RBCS3

Background. The aim of this project is to determine whether expression of G867 from a RBCS3 promoter, which predominantly drives expression in photosynthetic tissue, is sufficient to confer stress tolerance that is similar to, or better than, that seen in 35S::G867 lines, while eliminating some of the undesirable morphologies associated with constitutive G867 overexpression.

Morphological Observations. Arabidopsis lines in which G867 was expressed from the RBCS3 promoter (using the two component system) exhibited no consistent alterations in growth and development. The majority of plants showed wild-type morphology, though a number of RBCS3::G867 lines were noted to have a slight reduction in overall size. In general, the effects seen were less severe that those obtained with 35S::G867 lines.

T1 lines generally appeared wild type at all developmental stages. Some size variation was apparent at the rosette stage, with some plants being small to varying extents. Four lines developed slowly, had slightly contorted leaves, and bolted slightly later than controls.

Three T2 lines were examined; plants in each of these populations displayed a wild-type phenotype.

Physiology (Plate assays) Results. Four RBCS3::G867 lines out of ten performed better than wild-type seedlings in a germination assay in the presence of sodium chloride.

Discussion. We have isolated RBCS3::G867 lines via a 2-component approach. The RBCS3 lines were tested in drought related assays; moderate salt tolerance was seen in plate-based assays, but no clear advantage over controls was observed in soil based clay pot assays

Potential applications. Based on the data from overexpression studies, G867 is a good candidate gene for improving stress tolerance in commercial species. However, given the undesirable morphologies that arise from G867 overexpression (see the 35S::G867 report), it might be necessary to optimize the gene by use of alternative promoters or sequence modifications before it can be used to develop products. The results of this experiment indicate that use of the RBCS3 promoter may be a potential means to achieve this. The stress tolerance phenotypes obtained with this construct were less compelling than with the 35S::G867 combination.

G867 (SEQ ID NO: 87 and 88; Arabidopsis thaliana)—Super Activation (N-GAL4-TA)

Background. The aim of this project was to determine whether the efficacy of the G867 protein could be improved by addition of an artificial GAL4 activation domain.

Morphological Observations. Overexpression of a super-active form of G867, comprising a GAL4 transactivation domain fused to the N terminus of the protein, produced no consistent effects on Arabidopsis morphology.

Two batches of lines containing construct P21201 have were obtained: lines 981-991 and 1141-1160. The majority of these T1 lines and each of three T2 populations appeared wild type at all developmental stages.

Physiology (Plate assays) Results. Four out of ten 35S::GAL4-G867 lines were more tolerant to sodium chloride than controls in a germination assay.

Physiology (Soil Drought-Clay Pot) Summary. Three independent 35S::GAL4-G867 lines were tested in soil drought assays.

A single line #981 showed significantly better survival than controls in a single run of a split pot assay and in a first run of a whole pot assay. In a second run of the whole pot assay, the line showed a comparable response to wild type.

A second line #987 showed a wild-type performance in the split pot assay and inconsistent results in the whole pot assay. In one planting, this line performed substantially better than controls, but in a subsequent planting, where the plants suffered a harsher drought treatment, line #987 showed a worse performance than controls.

The third line #1148, performed worse in two repeats of the whole pot assay and showed a wild-type response in the split pot assay.

TABLE 61
35S::GAL4-G867 drought assay results:

| | | Mean | | p-value for | Mean | Mean | |
|---|---|---|---|---|---|---|---|
| | Project | drought | Mean drought | drought score | survival | survival | p-value for difference in |
| Line | Type | score line | score control | difference | for line | for control | survival |
| 981 | GAL4 | 0.60 | 0.60 | 0.90 | 0.19 | 0.14 | 0.26 |
| | N-term | | | | | | |
| 981 | GAL4 | 1.0 | 0.40 | 0.092* | 0.77 | 0.17 | 0.000000000000000000027* |
| | N-term | | | | | | |
| 981 | GAL4 | 0.58 | 0.17 | 0.089* | 0.11 | 0.048 | 0.089* |
| | N-term | | | | | | |

Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have isolated lines that overexpress a version of the G867 protein that has a GAL4 activation domain fused to the N terminus. These transformants showed no consistent differences in morphology compared to wild type controls. This result contrasts the effects of overexpression of the wild-type form of the G867 protein, as well as with the C-terminal GAL4 fusion, which both produced a marked reduction in overall size and other developmental abnormalities (i.e., as compared with 35S::G867 and G867 C-terminal fusions).

Potential applications. Based on the data from overexpression studies, G867 is a good candidate gene for improving stress tolerance in commercial species. However, given the undesirable morphologies that arise from G867 overexpression (i.e., as compared to 35S::G867 lines), it might be necessary to optimize the gene by use of alternative promoters or sequence modifications before it can be used to develop products. The morphology and physiology results from this experiment indicate addition of an artificial activation domain at the N-terminus may be one such modification. However, the levels of stress tolerance obtained with the GAL4-G867 fusion did not appear better than those conferred by the native form of the protein

G867 (SEQ ID NO: 87 and 88; Arabidopsis thaliana)—Super Activation (C-GAL4-TA)

Background. The aim of this project was to determine whether the efficacy of the G867 protein could be improved by addition of an artificial GAL4 activation domain.

Morphological Observations. Overexpression of a super-active form of G867, comprising a GAL4 transactivation domain fused to the C terminus of the protein, produced complex effects on Arabidopsis morphology.

Two batches of lines containing construct P21193 were obtained: 521-531 and 641-645. The majority of these plants appeared wild type. However, a number of lines (522, 523, and 525) from the first batch were noted to be small at early stages of development, while the second batch all appeared wild type. Previously, we concluded that overexpression of this construct yielded no consistent effects on morphology. However, on examining three T2 populations, a more distinct (but pleiotropic) phenotype became apparent.

T2-523: all plants were small at early stages, had rather shiny leaves, showed an acceleration in the onset of flowering, had rather bushy inflorescences, and floral abnormalities.

T2-525; 2/6 plants slightly large at early stages and grew more rapidly than controls. 4/6 rather small and slow developing.

T2-528: all plants small, slightly dark with shiny leaves. 3/6 flowered slightly early. Floral abnormalities and poor fertility were apparent.

Physiology (Plate assays) Results. Three of ten 35S::G867-GAL4 lines performed better than wild-type on plates containing sucrose in a germination assay. Two of these lines were also less sensitive to ABA in another germination assay, and three lines showed enhanced performance in a chilling growth assay. Two lines were also small and pale.

Discussion. We have isolated lines that overexpress a version of the G867 protein that has a GAL4 activation domain fused to the C terminus. Three of the ten lines were substantially more resistant to sucrose in germination assays than controls. The same three lines also out-performed wild type to varying extents in germination assays on ABA and in growth assays under cold conditions. Interestingly, though, these three lines performed worse than controls in clay pot soil drought assays. These same three lines were smaller and darker than wild-type controls, showed altered flowering time and had mild fertility problems. Such effects were very similar to those seen in 35S::G867 lines. The remaining seven lines, however, displayed a wild-type response in plate-based assays. A number of these transformants showed a slight reduction in size, but the majority showed no consistent differences in morphology compared to wild type controls. This result contrasts the effects of overexpression of the wild-type form of the G867 protein, which produces a marked reduction in overall size and other developmental abnormalities.

It appears that while the additional domain added at the C-terminus reduces deleterious phenotypes associated with overexpression of G867, stress resistance phenotypes are also seen at lower frequency in these 35S::G867-GAL4 lines (e.g., as compared to the non-modified 35S::G867, where all of the lines tested showed abiotic stress resistance.

Potential applications. Based on the data from overexpression studies, G867 is a good candidate gene for improving stress tolerance in commercial species. However, given the undesirable morphologies that arise from G867 overexpression, it might be necessary to optimize the gene by use of alternative promoters or sequence modifications before it can be used to develop products. The results of this experiment suggest addition of an artificial activation domain at the C-terminus does not offer any consistent improvement relative to the native form of the protein.

G867 (SEQ ID NO: 87 and 88; Arabidopsis thaliana)—Deletion Variant

Background. The aim of this project was to further refine our understanding of G867 function by use of a dominant negative approach in which a truncated version of the protein was overexpressed. Two constructs were built; one of these overexpressed a short version of the G867 protein comprising the AP2 domain, whereas the other was a truncated version comprising the B3 domain, but not the AP2 domain.

Morphological Observations. Lines have been obtained for each of two different G867 deletion constructs: P21275 and P21276.

Lines 1041-1060 and 1441-1460 were transformed with P21275, a construct in which a truncated version of G867 comprising the AP2 domain was overexpressed. Plants harboring this construct exhibited no consistent differences in morphology to wild-type controls.

Lines 881-889, lines 1001-1016, and lines 1361-1380 contained P21276, a construct in which a truncated version of G867 containing the B3 domain, but not the AP2 domain, was overexpressed. Plants from each of these three sets of lines showed a number pleiotropic but distinct alterations in morphology. The plants generally formed narrow strap like leaves, were slightly reduced in overall size, had reductions in trichome density, showed increased activity of secondary shoot meristems (in the primary rosette leaf axils), and had abnormalities in shoot phyllotaxy. Some of the lines were also noted to flower early and develop rather more rapidly than wild type.

The above phenotypes were observed to varying extents in 6/9 lines from the 881-889 set: (#884, 885, 886, 887, 888, 889), 16/16 lines from the 1001-1016 set, and 19/20 lines (all except #1371) from the 1361-1380 set. Two T2 lines (T2-881 and T2-889, T2-1002) were also examined; plants from each of these populations were early flowering and formed rather bushy stems.

Physiology (Plate assays) Results. Two different G867 deletion constructs (P21275 and P21276) have been analyzed in abiotic stress assays.

Lines 886 to 1014 contained P21276, a construct in which a truncated version of G867 containing the B3 domain, but not the AP2 domain, was overexpressed. Three lines were more tolerant to cold stress during germination.

Lines 1041 to 1060 and 1441-1460 were transformed with P21275, a construct in which a truncated version of G867 comprising the AP2 domain was overexpressed. Five of twenty lines were more tolerant compared with wild-type plants in a growth assay in the presence of chilling temperatures. Four of the twenty lines performed better than controls in a severe dehydration assay.

Discussion. We have now isolated lines harboring each of the G867 dominant negative constructs. Lines containing the construct for overexpression of the AP2 domain exhibited wild-type morphology. This contrasts the effects of overexpressing the full-length G867 protein, which causes a number of undesirable morphological changes. Thus, the regions of the G867 protein that cause undesirable morphologies are potentially external to the AP2 domain itself.

Lines carrying the other construct, expressing a truncated form of the protein containing the B3 domain, showed a variety of morphological alterations including changes in phyllotaxy, leaf shape, overall size, and flowering time. Such pleiotropic effects are rather difficult to interpret, but suggest that G867 can impact a range of developmental processes. In particular, some of the lines showed a reduction in trichome density, indicating that G867 can affect the genetic pathways that specify trichome development.

Physiological assays have been performed on lines carrying each of the two constructs. Neither type of line performed differently than controls in soil-based drought tolerance assays. Plate-based assays indicate that both types of construct confer moderate cold tolerance, either in germination or growth.

Potential applications. The morphological changes seen in dominant negative lines overexpressing the B3 domain indicate that G867 can be used to manipulate various aspects of plant development. In particular, the gene may be used to modify trichome formation. Such structures have a variety of roles and in some species accumulate potentially valuable secondary metabolites. In other cases trichomes are thought to offer protection against water loss or insect attack. These dominant negative lines may also be used to confer cold tolerance.

G867 (SEQ ID NO: 87 and 88; Arabidopsis thaliana)—RNAi (clade)

Background. The aim of this project was to further refine our understanding of G867 function by use of an RNAi approach; two constructs (see sequence section) were generated that were targeted towards reducing activity of all members of the G867 clade. Given that the different members of the G867 clade are potentially functionally redundant, it was thought that this method could reveal phenotypes that might not be visible in single KO lines for the individual clade members.

Morphological Observations. Lines for two different G867-RNAi (clade) constructs have been examined. Some evidence of delayed flowering and increased rosette size was apparent, but these phenotypes were obtained at a relatively low frequency.

Line Details:

(N.B. P21303 and P21304 were different constructs. P21162, however, was identical to P21303. See sequence section for details.)

P21303 and P21162 Lines:

T1 lines 421-429: all were slightly small at early stages. 2/9 lines (422, 426) were rather late flowering.

T2-422, T2-426, T2-427: all appeared wild type.

T1 lines 1201-1217. 3/17 (1202, 1203, 1205) developed large rosettes with long leaves and petioles were slightly late flowering. Others appeared wild type.

T2-1202: all were slightly late flowering.

T2-1203, T2-1205: all appeared wild type.

T1 lines 1661-1680: some size variation, with many lines being slightly small at early stages. 3/20 lines were rather late developing versus controls. Others appeared wild type at later stages.

T2-1674: all appeared wild type.

T2-1673: all had slightly large rosettes at late stages.

T2-1665: all were slightly large at the rosette stage.

P21304 Lines:

T1 lines 1221-1240: no consistent differences to controls.

T2-1240: all appeared wild type.

T2-1239: 2/6 showed slightly enlarged rosettes, 4/6 were wild type.

T2-1235: 2/6 showed slightly enlarged rosettes, 4/6 were wild type.

Physiology (Plate assays) Results. Lines for two different G867-RNAi (clade) constructs were tested in plate based assays. Overall, although sporadic “hits” were obtained in some of the assays, lines for either of these constructs showed no consistent differences in performance relative to controls under stress conditions.

A number of the lines carrying P21303, however, were noted to be larger and more vigorous at the seedling stage relative to controls.

(N.B. P21303 and P21304 were different constructs. P21162, however, was identical to P21303.

Physiology (Soil Drought-Clay Pot) Summary. Lines for two different G867-RNAi (clade) constructs were tested in soil drought assays.

Two of three lines harboring P21162 showed an enhanced survival relative to controls in a single run of a split pot soil drought assay (lines and control together in same pot).

Three lines for another construct (P21304) were also tested in a split pot assay. All of these lines showed a comparable rate of survival versus wild-type, but one lines showed less severe stress symptoms versus the control at the end of the drought period.

TABLE 62
G867-RNAi (clade) drought assay results:

| | | | Mean | Mean | | | | |
|---|---|---|---|---|---|---|---|---|
| | | | drought | drought | p-value for | Mean | Mean | p-value for |
| | | | score | score | drought score | survival | survival for | difference |
| PID | Line | Project Type | line | control | difference | for line | control | in survival |
| P21162 | 422 | RNAi (clade) | 1.6 | 1.6 | 1.0 | 0.23 | 0.29 | 0.47 |
| P21162 | 426 | RNAi (clade) | 2.0 | 2.0 | 1.0 | 0.29 | 0.15 | 0.041* |
| P21162 | 427 | RNAi (clade) | 2.8 | 2.8 | 1.0 | 0.39 | 0.19 | 0.0049* |

Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have now isolated lines harboring each of the G867 RNAi clade constructs. The plants displayed some changes in morphology, at low frequency, relative to wild-type controls, including alterations in plant size and flowering time. Two lines survived drought better than controls in soil drought assays. One of these lines (#426) was more tolerant to salt in plate-based assays, and second line (#427), was more tolerant to severe dehydration and grew better than controls in plate-based assays.

Potential applications. Based on the data from overexpression studies, G867 is a good candidate for improving stress tolerance in commercial species. However, this RNAi project has not provided much additional insight into the native role of the G867 clade. The results do indicate, though, that a knock-down approach on the G867 related genes can be used as a means to enhance stress tolerance and modify developmental processes.

G9 (SEQ ID NO: 105 and 106; Arabidopsis thaliana)—Constitutive 35S

Background, G9 is a paralog of G867, and has been referenced in the public literature as RAP2.8 and RAV2 (Okamuro et al., 1997; Kagaya et al., 1999). G9 has been reported as a putative ABA agonist in maize protoplasts (Gampala et al. 2004). Previously, we observed that G9 overexpression enhanced root growth. The aim of this study was to re-assess 35S::G9 lines and determine whether overexpression of the gene could confer enhanced stress tolerance in a comparable manner to G867. We also sought to test whether use of a two-component overexpression system would produce any strengthening of the phenotype relative to the use of a 35S direct promoter-fusion.

Morphological Observations. G9 overexpression lines were generated containing the 35S direct promoter fusion construct (P167, lines 301-318), and via the two component system (6506, P7824, lines 461-473). Both these batches of 35S::G9 lines displayed a number of pleiotropic and variable alterations in overall morphology relative to wild-type controls. Such developmental changes included a reduction in overall size and alterations in leaf orientation. In some lines, changes in leaf shape, flowering time and non-specific floral abnormalities that reduced fertility were observed. Details of lines are shown below:

Direct fusion lines were generally smaller than controls with had narrow leaves and a reduction in rosette biomass. Some plants showed poor fertility and set very few siliques.

Three T2 lines were morphologically examined:

These plants were smaller than controls (in some cases, the difference was slight), slow developing, late bolting, had vertically oriented leaves and showed floral abnormalities.

T2-304: 6/6 were slightly small and showed delayed bolting.

Two component lines were small and showed vertically oriented leaves and slow growth relative to wild type. A number of plants were late developing.

Physiology (Plate assays) Results. 35S::G9 lines showed more root growth on control plates compared to wild-type control seedlings. When seedlings of ten new (direct fusion) lines overexpressing G9 were analyzed, increased tolerance to salt was observed in all ten lines. Several lines were also less sensitive to ABA and sucrose in separate germination assays. Tolerance to cold conditions was also observed in a growth assay under chilling conditions. Seedlings from some lines were also small and chlorotic. However, enhanced root growth was not observed on control plates, in this set of lines.

Tolerance to salt, sucrose and ABA was also seen when a two component expression system was used to drive the constitutive overexpression of G9. However, enhanced root hair production was noted in a few lines, in contrast to the results seen with the most recent set of direct promoter fusion lines.

Discussion. New overexpression lines have been obtained using both a direct promoter fusion construct and a two component expression system. Lines generated by either of these methods exhibited similar phenotypes and displayed a number of morphological effects that had not been observed during our earlier genomics screens. These included a reduction in overall size, alterations in leaf orientation (which potentially indicated a disruption in circadian control), slow growth, and floral abnormalities relative to controls.

It should be emphasized that we have obtained comparable developmental effects as well as a strong enhancement of abiotic stress tolerance from all four of the Arabidopsis genes in the G867 study group (G9, G867, G993, and G1930). The almost identical phenotypic effects produced by these genes strongly indicate that they are functionally equivalent.

Potential applications. Based on the results of our overexpression studies, G9 and its related paralogs are excellent candidate genes for improvement of abiotic stress tolerance in commercial species. However, the morphological effects associated with their overexpression suggest that tissue-specific or conditional promoters might be required to optimize the utility of these genes.

G993 (SEQ ID NO: 89 and 90; Arabidopsis thaliana)—Constitutive 35S

Background. G993 is a paralog of G867. Previously, we observed that G993 overexpression lines exhibited a number of morphological abnormalities. The aim of this study was to re-assess 35S::G993 transformants using a greater number of lines and determine whether overexpression of the gene could confer enhanced stress tolerance in a comparable manner to G867.

Morphological Observations. Additional G993 overexpression lines have now been generated using both the two-component system and a direct fusion approach.

35S::G993 plants displayed a number of pleiotropic and variable alterations in overall morphology relative to wild-type controls. Such developmental changes included a reduction in overall size, alterations in leaf shape, alternations in hypocotyl length and cotyledon orientation, slower growth, altered flowering time and non-specific floral abnormalities that reduced fertility were observed.

Direct promoter-fusion lines were generally small and slow developing, with floral abnormalities and poor fertility.

Two-component lines (generated using the two component vectors P6506 and P21149) showed particularly severe phenotypes; the majority were extremely dwarfed, dark, and slow developing, and yielded few if any seeds. Consequently, the 2-component lines could not be tested in physiology assays.

Physiology (Plate assays) Results. When seedlings overexpressing G993 were examined, eight of these lines gave positive stress tolerance phenotypes in one or more of the following assays: salt ( 6/10), sucrose 5/10), cold germination ( 3/10) or growth under chilling conditions ( 6/10).

Interestingly, two lines which did not show a positive result on the stress plates (#330 and #337) showed a dramatic increase in root hair density when grown on regular control MS plates.

Discussion. New overexpression lines have been obtained using both a two-component and a direct fusion approach. These lines exhibited similar phenotypes to those observed during our earlier genomics studies and were generally small, slow developing, and poorly fertile. Occasional lines also showed features that indicated a disruption in light regulated development, such as long hypocotyls and alterations in leaf orientation. Our attempts to generate 2-component overexpression lines resulted in dwarfed, dark, and slow developing plants, which were too infertile to produce enough seeds for physiological testing.

It should be emphasized that we have obtained comparable developmental effects as well as a strong enhancement of drought related stress tolerance in all four of the Arabidopsis genes in the G867 study group (G9, G867, G993, and G1930). The almost identical phenotypic effects produced by these genes strongly indicate that they are functionally equivalent.

Potential applications. Based on the results of our overexpression studies, G993 and its related paralogs are excellent candidate genes for improvement of drought related stress tolerance in commercial species. However, the morphological effects associated with their overexpression, suggests that tissue specific or conditional promoters might be required to optimize the utility of these genes.

Additionally, the increased root hair production seen in 35S::G993 lines indicates that the gene may be used to enhance root growth and differentiation and might thereby improve performance under other stresses, such as low nutrient availability.

G1930 (SEQ ID NO: 91 and 92; Arabidopsis thaliana)—Constitutive 35S

Background. G1930 is a paralog of G867. Previously, we observed that G1930 overexpression lines exhibited increased tolerance to sodium chloride, ABA, and sucrose. The plants also showed a number of morphological abnormalities. The aim of this study was to re-assess 35S::G1930 transformants using a greater number of lines and determine whether overexpression of the gene could confer enhanced stress tolerance in a comparable manner to G867. We also sought to test whether use of a two-component overexpression system would produce any strengthening of the phenotype relative to the use of a 35S direct promoter-fusion.

Morphological Observations. New sets of G1930 overexpression lines have now been generated, containing the 35S direct promoter fusion construct (P1310, lines 301-318, 361-371), and via the two component system (P6506, P3373, lines 321-340)

These batches of 35S:G1930 lines displayed a number of pleiotropic alterations in overall morphology relative to wild-type controls. Such developmental changes included a reduction in overall size and alterations in leaf orientation. In some lines, changes in leaf shape, hypocotyl length, trichome density, flowering time and non-specific floral abnormalities that reduced fertility were also observed. Details of lines are provided below:

Direct fusion lines were distinctly small with narrow leaves that were positioned in an upright manner. Some plants displayed abnormal flowers in which buds often fail to open. Some lines had reduced trichome density and showed poor fertility.

Two component lines were small and at the seedling stage showed rather long hypocotyls. A number of lines displayed vertically oriented leaves and bolted later than wild type. A number of lines displayed flowers that failed to properly open and were of poor fertility.

Physiology (Plate assays) Results. In the initial genomics program, G1930 overexpressors were more tolerant of osmotic stress conditions than controls. The plants responded to high NaCl and high sucrose on plates with more seedling vigor compared to wild-type control plants.

This observation was confirmed when seedlings of several lines overexpressing G1930 by direct promoter fusion were re-examined. Most of the lines tested were more tolerant to sodium chloride, sucrose in a germination assay, or tolerant to cold in a growth assay than controls. While many 2-component lines were tolerant to ABA during germination, none of the direct promoter fusion lines showed this tolerance.

Discussion. We generated additional 35S::G1930 lines via both the direct promoter-fusion and the 2-component methods. Both types of lines exhibited a variety of morphological phenotypes including reduced size, slow growth, and alterations in leaf orientation. In some lines, changes in leaf shape, hypocotyl length, trichome density, flowering time and non-specific floral abnormalities that reduced fertility were also observed.

It should be emphasized that we have obtained comparable developmental effects as well as a strong enhancement of drought related stress tolerance from all four of the Arabidopsis genes in the G867 study group (G9, G867, G993, and G1930). The almost identical phenotypic effects produced by these genes strongly indicate that they are functionally equivalent.

Potential applications. Based on the results of our overexpression studies, G1930 and its related paralogs are excellent candidate genes for improvement of abiotic stress tolerance in commercial species. However, the morphological effects associated with their overexpression, suggests that tissue specific or conditional promoters might be required to optimize the utility of these genes.

G3389 (SEQ ID NO: 103 and 104; Oryza sativa)—Constitutive 35S

Background. G3389 is a rice gene that is a closely-related homolog of G867. The aim of this project is to determine whether G3389 has an equivalent function to G867 via the analysis of 35S::G3389 Arabidopsis lines.

Morphological Observations. 35S::G3389 lines were obtained at relatively low frequency, a total of only five transformants were obtained from three different selection attempts. This suggests that the gene might be lethal when overexpressed at high levels.

In the T1 generation, all four of the five lines (#341, 342, 343, and 344) were early flowering and all of the lines except #342 were smaller than wild type. Two of the T1 plants (341 and 344 had rather bushy inflorescences). One of the lines (#345) was extremely tiny.

Four of the lines were then examined in the T2 generation; three of these populations appeared wild type, but plants from T2-341 displayed accelerated flowering and had bushy inflorescences, similar to what had been observed in the T1 generation.

Physiology (Plate assays) Results. All four lines had good performance in germination assays relative to controls in the presence of sodium chloride or cold temperatures. Two of these lines were tolerant to heat during growth assays.

Physiology (Soil Drought-Clay Pot) Summary. One 35S::G3389 line showed greater drought tolerance than wild type.

TABLE 63
35S::G3389 drought assay results:

| | | Mean | | p-value for | Mean | | p-value for |
|---|---|---|---|---|---|---|---|
| | Project | drought | Mean drought | drought score | survival for | Mean survival | difference in |
| Line | Type | score line | score control | difference | line | for control | survival |
| 341 | DPF | 1.5 | 0.78 | 0.068* | 0.13 | 0.13 | 0.93 |

DPF = direct promoter fusion
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. In general, 35S::G3389 plants displayed comparable, but weaker developmental and stress tolerance effects to the 35S::G867 plants, indicating that this ortholog is at least partially functionally equivalent to the G867 study group in Arabidopsis. Relatively few transformants were obtained with multiple attempts, however, suggesting that the gene might be lethal when overexpressed at high levels.

Potential applications. Based on the results of our overexpression studies, G3389 is a candidate gene for improvement of abiotic stress tolerance in commercial species. However, the possible deleterious effects associated with high overexpression levels, suggest that tissue specific or conditional promoters might be required to optimize the utility of this gene. The changes in flowering time and morphology seen in 35S::G3389 lines indicate that this gene could be used to modify the floral transition and/or plant development.

G3391 (SEQ ID NO: 93 and 94; Oryza sativa)—Constitutive 35S

Background. G3391 is a rice gene that is a closely-related homolog of G867. The aim of this project was to determine whether G3391 has an equivalent function to G867 via the analysis of 35S::G3391 Arabidopsis lines.

Morphological Observations. Overexpression of G3391 produced a variety of pleiotropic morphological effects in Arabidopsis. 35S::G3391 lines were distinctly small and showed alterations in leaf shape, leaf orientation, flowering time, and floral defects that resulted in poor fertility. Three sets of lines were obtained, as detailed below:

Lines 321-335: all T1 lines were markedly small, with narrow pointed leaves. #321, 328, 335 were tiny and perished at early stages. #322, 323, 327, 329, 332, 334 were early flowering. All lines had very poor seed yield. Two T2 populations were also examined; T2-326 plants were early flowering and had rather upright leaves. T2-324 plants showed some size variation, but were otherwise wild type.

Lines 361-374: all T1 lines were tiny and dark in coloration at the seedling stages. Later these plants were small with pointed upright leaves. #362, 365, 368 were very small. #363, 366, 369, 371, 372 were early flowering. All lines showed poor fertility and yielded relatively few seeds.

Lines 381-384: all T1 lines were small, with spindly stems and showed slightly early flowering.

Physiology (Plate assays) Results. Three 35S::G3391 lines were more tolerant to sodium chloride in a germination assay. It is worth noting that five of the lines that were not tolerant to sodium chloride were small, vitrified, or chlorotic.

Discussion. 35S::G3391 lines exhibited a number of morphological changes similar to those seen in 35S::G867 lines including a reduction in overall size, alterations in leaf shape and orientation, and floral abnormalities. Additionally, a substantial number of the G3391 lines showed accelerated flowering, indicating that G3391 acts to promote the floral transition. Other lines showed small, vitrified, or chlorotic phenotypes and were not salt tolerant in the plate-based assays. No consistent alteration in tolerance was obtained in a soil drought experiment.

Potential application: This project indicates that G3391 could be applied to effect abiotic stress tolerance in commercial species. In addition, the accelerated flowering seen in 35S::G3391 plants indicate that the gene could be used to manipulate flowering time. In particular, shortening generation times would also help speed-up breeding programs, particularly in species such as trees, which typically grow for many years before flowering. Conversely, it may be possible to modify the activity of G3391 or its closely-related homologs to delay flowering in order to achieve an increase in biomass and yield. The possible deleterious effects associated with high overexpression levels, however, suggests that tissue specific or conditional promoters might be required to optimize the utility of this gene.

G3432 (SEQ ID NO: 101 and 102; Zea mays)—Constitutive 35S

Background. G3432 is a maize gene that is a closely-related homolog of G867. The aim of this project was to determine whether G3432 has an equivalent function to G867 via the analysis of 35S::G3432 Arabidopsis lines.

Morphological Observations. Overexpression of G3432 produced a variety of deleterious pleiotropic morphological effects in Arabidopsis. 35S::G3432 lines were extremely small and showed alterations in coloration, leaf shape, leaf orientation, and floral defects that resulted in poor fertility. Occasional lines also showed reductions in trichome density. Many lines were late flowering. It should be noted that only lines with a relatively weak phenotype yielded sufficient seed for physiology assays.

Physiology (Soil Drought—Clay Pot) Summary. Three independent 35S::G3432 lines were tested in a single run of a soil drought assay. One of these lines (#304) showed a significantly improved survival relative to wild type. However, another line (#312) showed a worse survival than wild type. The third line (#309) showed a comparable performance to controls.

It has not been determined whether there exists a molecular basis (such as differences in transgene expression level) for the opposing results obtained with the different lines. It should be noted, though, that plants from line 312 were extremely tiny and this likely influenced their performance.

TABLE 64
35S::G3432 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 304 | DPF | 3.5 | 1.0 | 0.0056* | 0.57 | 0.16 | 0.0000000027* |

DPF = direct promoter fusion
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3432 lines exhibited a number of morphological changes including a reduction in overall size, alterations in leaf shape and orientation, and floral abnormalities, and in some cases, reduced trichome density. These phenotypes were more severe than, but otherwise comparable to, those obtained from overexpression of G867. These lines have now been tested in drought related stress assays. One line, #304, performed significantly better than controls in the clay pot drought assay, while another line, with a more severe morphological phenotype, performed worse than controls in the same assay. In the plate-based assays, all of the 35S::G3432 showed severe morphological defects versus controls, which made the results difficult to interpret.

Potential application: We have obtained initial evidence from soil assays that G3432 can effect drought tolerance. Nevertheless, the undesirable effects associated with overexpression of this gene suggest that it would need to be optimized by using a different promoter or by engineering changes within the protein, before it could be applied to develop a product.

The morphological changes seen in 35S::G3432 lines indicate that the gene may be used to manipulate various aspects of plant development. In particular, G3432 may be used to modify trichome formation. Such structures have a variety of roles and in some species accumulate potentially valuable secondary metabolites. In other cases trichomes are thought to offer protection against water loss or insect attack.

G3451 (SEQ ID NO: 107 and 108; Glycine max)—Constitutive 35S

Background. G3451 is a soy gene that is a closely-related homolog of G867. The aim of this project was to determine whether G3451 has an equivalent function to G867 via the analysis of 35S::G3451 Arabidopsis lines.

Morphological Observations. Overexpression of G3451 produced a wide spectrum of effects on Arabidopsis growth and development, including alterations in plant size, coloration, growth rate, leaf shape and orientation, and flowering time.

Three batches of T1 lines were obtained (301-317, 321-335, 341-360). At the early seedling stage, some of these plants had cotyledon abnormalities. Later, many lines were slow growing, exhibited vertically oriented leaves and flowered later than wild-type. Such phenotypes were apparent in all the lines, to varying extents. It should be noted that the most severely affected lines died without yielding seed; only lines with a mild phenotype could therefore be taken forward for physiology assays.

Three T2 populations were morphologically examined; the plants were rather dwarfed, but the phenotypes were generally weaker than those seen among primary transformants. T2-303 plants were also slightly early flowering.

Physiology (Plate assays) Results. Six out of ten 35S::G3451 lines showed good performance when germinated on plates containing sucrose. In addition, seven out of ten lines were noted to have small pale seedlings with narrow leaves and had short thick roots with more root hairs.

Physiology (Soil Drought-Clay Pot) Summary. G3451 overexpression conferred enhanced drought tolerance in Arabidopsis. Three independent 35S::G3451 lines were tested; two of these lines (#302 and 314) showed significantly better survival than the wild type control. The third line (#303) was significantly less stressed than the control after 8 days of dry down, but did not show a significant difference in survival rate.

TABLE 65
35S::G3451 drought assay results:

| | | | Mean | | | | |
|---|---|---|---|---|---|---|---|
| | | Mean | drought | p-value for | Mean | Mean | |
| | Project | drought | score | drought score | survival | survival for | p-value for difference in |
| Line | Type | score line | control | difference | for line | control | survival |
| 302 | DPF | 3.2 | 0.67 | 0.0020* | 0.57 | 0.14 | 0.000000010* |
| 303 | DPF | 1.8 | 0.67 | 0.031* | 0.21 | 0.14 | 0.18 |
| 314 | DPF | 4.5 | 0.67 | 0.0013* | 0.75 | 0.14 | 0.0000000000000036* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. A number of 35S::G3451 lines have been established, and show a number of morphological defects including a reduction in plant size, unusual coloration, slow growth rate, alterations in leaf shape and orientation, and slightly early flowering time. Many of these phenotypes are similar to what was observed in 35S::G867 lines, indicating that this soy homolog has some conserved function with the Arabidopsis gene. 35S::G3451 lines with mild morphological phenotypes were tested for abiotic stress tolerance. As with some of the other soy homologs of G867, a root hair phenotype is visible in these overexpression lines.

Potential application: Based on the results of our overexpression studies, G3451 is an excellent candidate gene for improvement of abiotic stress tolerance in commercial species. However, the morphological effects associated with its overexpression, suggests that tissue specific or conditional promoters might be required to optimize the utility of this gene.

G3452 (SEQ ID NO: 97 and 98; Glycine max)—Constitutive 35S

Background. G3452 is a soy gene that is a closely-related homolog of G867. The aim of this project was to determine whether G3452 has an equivalent function to G867 via the analysis of 35S::G3452 Arabidopsis lines.

Morphological Observations. Overexpression of G3452 produced a wide spectrum of effects on Arabidopsis growth and development, including alterations in plant size, coloration, growth rate, leaf shape and orientation, and flowering time.

Two batches of T1 lines were obtained (301-318 and 321-340). The majority of plants were small, dark, slow developing, had vertically oriented abnormally-shaped leaves and flowered later than wild type. However, a small number of lines were noted to be slightly early flowering. Some lines also exhibited floral defects and aberrant branching patterns. The above effects were also apparent in two (T2-305 and T2-310) of three T2 populations that were morphologically examined. Plants from T2-304 appeared wild type.

Physiology (Plate assays) Results. Five of six 35S::G3452 lines were more tolerant to sucrose in a germination assay. Four of these lines were more tolerant to sodium chloride in another germination assay, and two of the lines performed well in a cold germination assay. Several 35S::G3452 lines were noted to be small and chlorotic with little secondary root growth. However, two lines showed more root hair growth versus controls.

Discussion. 35S::G3452 lines displayed a number of morphological similarities to those seen in 35S::G867 lines, such as reduced plant size, leaf coloration, growth rate, leaf shape and orientation. A number of the 35S::G3452 lines also showed alterations in flowering time. 35S::G3452 lines were subjected to drought related assays, and were more tolerant to sucrose, sodium chloride, and cold during germination than control plants. As with other soy homologs of G867, a root hair phenotype is visible in these overexpression lines.

Potential application: This project indicates that G3452 could be applied to effect drought-related abiotic stress tolerance in commercial species. In addition, the accelerated flowering seen in 35S::G3452 plants indicate that the gene could be used to manipulate flowering time. In particular, shortening generation times would also help speed-up breeding programs, particularly in species such as trees, which typically grow for many years before flowering. Conversely, it may be possible to modify the activity of G3452 or its closely-related homologs to delay flowering in order to achieve an increase in biomass and yield. The gene might also be used to modify other aspects of development such as root morphology.

G3455 (SEQ ID NO: 95 and 96; Glycine max)—Constitutive 35S

Background. G3455 is a soy gene that is a closely-related homolog of G867. The aim of this project was to determine whether G3455 has an equivalent function to G867 via the analysis of 35S::G3455 Arabidopsis lines.

Morphological Observations. Overexpression of G3455 produced a complex spectrum of morphological effects including changes in plant size, flowering time, leaf shape and orientation, trichome density, and floral defects.

A total of twenty T1 lines (361-380) were examined; at early seedling stages, about 50% of the lines were small and dark in coloration. Later, the majority of plants were smaller then wild-type, to varying extents. 6/20 (#362, 364, 370, 371, 375, 377) lines were early flowering and 6/20 had upright leaves (#365, 367, 368, 369, 374, 376). Two of the T1 lines exhibited a partial glabrous phenotype and were noticeably slow developing.

Three 35S::G3455 T2 populations were later examined. Plants from each of these populations were small, slow developing and displayed vertically oriented leaves. At later stages, the plants formed rather bushy inflorescences and had poorly developed siliques Physiology (Plate assays) Results. Nine out of ten 35S::G3455 lines performed well in a germination assay (versus controls) in the presence of sucrose. Several lines were also noted to have poor germination rates, long narrow leaves, less root branching, and in a few lines, more root hairs.

Discussion. A number of 35S::G3455 lines have been established, and show a number of morphological defects including changes in plant size, flowering time, leaf shape and orientation, trichome density, dark leaf coloration and slightly early flowering time. Many of these phenotypes are similar to what was observed in 35S::G867 lines, indicating that this soy homolog has some conserved function with the Arabidopsis gene. Almost all lines tested are more tolerant to sucrose than controls in the plate-based assays. As with other soy homologs of G867, a root hair phenotype is visible in these overexpression lines.

Potential applications. Based on the results of our overexpression studies, G3455 is a good candidate gene for improvement of abiotic stress tolerance in commercial species. However, the morphological effects associated with its overexpression, suggests that tissue specific or conditional promoters might be required to optimize the utility of this gene.

### The G922 Clade

G922 (SEQ ID NO: 327 and 328; Arabidopsis thaliana)—Constitutive 35S

Published Information

G922 corresponds to Scarecrow-like 3 (SCL3) first described by Pysh et al. (GenBank accession number AF036301; (1999) Plant J. 18: 111-119). Northern blot analysis results show that G922 is expressed in siliques, roots, and to a lesser extent in shoot tissue from 14 day old seedlings. Pysh et al did not test any other tissues for G922 expression. In situ hybridization results showed that G922 was expressed predominantly in the endodermis in the root tissue. This pattern of expression was very similar to that of SCARECROW (SCR), G306. Experimental evidence indicated that the co-localization of the expression is not due to cross-hybridization of the G922 probe with G306. Pysh et al proposed that G922 may play a role in epidermal cell specification and that G922 may either regulate or be regulated by G306.

The sequence for G922 can also be found in the annotated BAC clone F11F12 from chromosome 1 (GenBank accession number AC012561). The sequence for F11F12 was submitted to GenBank by the DNA Sequencing and Technology Center at Stanford University.

Experimental Observations. The function of this gene was analyzed using transgenic plants in which G922 was expressed under the control of the 35S promoter. Transgenic plants overexpressing G922 were more salt tolerant than wild-type plants as determined by a root growth assay on MS media supplemented with 150 mM NaCl. Plant overexpressing G922 also were more tolerant to osmotic stress as determined by germination assays in salt-containing (150 mM NaCl) and sucrose-containing (9.4%) media. G922 overexpressors were also more tolerant to growth in cold conditions than wild-type plants, and were also less sensitive to ABA than wild type. Morphologically, plants overexpressing G922 had altered leaf morphology (including curling, upright-oriented leaves), dark coloration, and somewhat reduced overall plant size. In wild-type plants, expression of G922 was induced by auxin, ABA, heat, and drought treatments. In non-induced wild-type plants, G922 was expressed constitutively at low levels.

The high salt assays indicated that this gene might confer drought tolerance, which was confirmed in soil-based assays, in which G922-overexpressors were significantly healthier after water deprivation treatment than controls.

Potential Applications. Based upon results observed in plants overexpressing G922 or its equivalogs could be used to increase tolerance to salt, drought and other hyperosmotic stress, increase tolerance to cold, and alter leaf morphology in plants.

### The G1073 Clade

G1073 (SEQ ID NO: 113 and 114; Arabidopsis thaliana)—Constitutive 35S

Background. G11073 was included in the drought program based on the drought tolerance and enhanced yield shown by 35S::G1073 lines. We have now designated this locus as HERCULES I (HRC1).

The aim of this study was to re-assess 35S::G1073 lines and compare its overexpression effects to those of its homologs. We also sought to test whether use of a two-component overexpression system would produce any strengthening of the phenotype relative to the use of a 35S direct promoter-fusion.

Morphological Observations. We have now generated 35S::G1073 lines (301-320) using the 2-component system. These lines exhibited comparable morphological effects to the 35S direct promoter fusion lines.

Two-Component Lines:

35S::G1073 two-component lines showed a mild to moderate delay in the onset of flowering and developed larger broader leaves than those of wild type. These effects were of intermediate penetrance, being observed to varying extents in eight of twenty T1 lines (#303, 304, 305, 308, 310, 314, 318, 319). It should be noted that considerable size variation was apparent among the remaining twelve lines in the set, and for unknown reasons, some of these plants (#307, 309, 312, 313, 315, 316, 317) died prior to reaching maturity.

The following lines were examined in subsequent generations, as detailed below:

T2-301: all showed a moderate phenotype.

T2-302: appeared wild type.

T2-304: all late flowering with broad curled leaves.

T3-304: all small at early stages with curled leaves. Plants were late flowering and leaves became enlarged at late stages.

T2-305: showed a mild phenotype.

T2-308: showed a mild phenotype.

T2-310: showed a moderate phenotype, all plants had distinctly broad leaves and flowered 3-4 days late. Inflorescence internodes were short and plants had a bushy appearance.

T2-311: occasional plants showed a very mild phenotype, most appeared wild type.

T2-314: all showed a moderately strong phenotype.

T2-319: showed a moderate phenotype, all plants had distinctly broad leaves and flowered about 1 week late.

Of the lines submitted for physiological assays, the following showed a segregation on selection plates in the T2 generation that was compatible with the transgene being present at a single locus: 305, 308, 310, 311, 314, 319. Lines 301, 304, 306, and 320 showed segregation that was compatible with insertions at multiple loci.

Direct Fusion Lines:

An additional set of direct fusion lines (1921-1940) harboring P448 have also been obtained: 6/20 of these lines (1923, 1927, 1929, 1933, 1935, 1937) showed large leaves and slightly delayed flowering. The remaining plants exhibited wild-type morphology.

A number of additional studies have been performed with the original 35S::G1073 lines obtained during the genomics program. We compared 35S::G1073 seedlings grown on plates with wild-type and noted that an increase in size is apparent from very early in the rosette stage. Additionally the 35S::G1073 seedlings have more extensive root development and develop longer root hairs than controls. Sections through the root tips of these lines, though, indicate that the root meristems have a comparable organization to those in the wild-type.

Results for a full-length G1073 clone. It should be noted that the initial G1073 constructs P448 (35S::G1073, Direct promoter-fusion) and P3369 (opLexA::G1073 for 2-components-supTfn) harbored an N-terminally truncated clone (see sequence details). We have now obtained a full-length G1073 clone (within P25703, a 35S::G1073 direct fusion construct). Two sets of lines for this fall-length clone have now been morphologically examined.

Lines 1961-1980 (for full-length clone P25703): 5/20 (#1968, 1969, 1972, 1975, 1978) showed a very marked phenotype (short petioles, large broad, round leaves, large flowers). 7/20 showed a moderate phenotype (1962, 1963, 1964, 1966, 1973, 1976, 1977), 4/20 showed a mild phenotype (1967, 1970, 1971, and 1974), and 4/20 (#1961, 1965, 1979, 1980) appeared wild type. The following lines were late flowering: #1963, 1966, 1968, 1969, 1972, 1975, 1977, 1978.

Lines 1981-2000 (for full-length clone P25703): 19/20 lines all showed evidence of an increased biomass phenotype (except for #1988 which was small with flat pale leaves). 4/20 (1982, 1987, 1990, 1992) lines displayed a mild phenotype, whereas the remaining lines showed a moderately strong phenotype. The following lines were late flowering: #1981, 1983, 1984, 1986, 1989, 1991, 1994, 1995, 1997, 1998, 2000.

Based on these two sets of lines, in comparison to the lines obtained for the N-terminally truncated form of G1073, it appears that the increased biomass phenotype is rather more marked and seen at higher penetrance when the full-length clone is overexpressed (during our earlier genomics program, 11/20 lines overexpressing the truncated form of G1073 showed an increased size phenotype).

Physiology (Plate assays) Results. 35S::G1073 lines behaved similarly to wild-type controls in all physiological assays performed. We have now re-examined a greater number of 35S::G1073 lines, this time via a 2-component overexpression approach. All ten of the new lines out-performed controls when germinated on plates containing sodium chloride. Five out of the ten lines also had better growth on plates containing sucrose.

A new set of 35S direct promoter fusion lines showed better tolerance to water stress in a severe dehydration stress assay, but did not exhibit better growth on plates containing sodium chloride or sucrose.

Physiology (Soil Drought-Clay Pot) Summary. 35S::G1073 two-component lines showed an enhanced performance, relative to controls, in soil drought assays. Five independent 35S::G1073 2-component lines have been tested. Two of these lines (#310 and 311) showed a significantly better survival than controls in each of two different repeats of a “whole pot” soil drought assay. Both of the lines, however, displayed a comparable performance to wild-type in a single run of the “split pot” assay (line and controls in same pot). A different, line (#314) was not tested in a “whole pot” assay, but survived better than controls in a “split pot” assay.

TABLE 66
35S::G1073 drought assay results:

| | | | | | p-value for | | | |
|---|---|---|---|---|---|---|---|---|
| | | | | Mean | drought | Mean | Mean | p-value for |
| | Project | Assay | Mean drought | drought | score | survival | survival | difference in |
| Line | Type | Type | score line | score control | difference | for line | for control | survival |
| 310 | TCST | Whole Pot | 3.0 | 1.4 | 0.0017* | 0.35 | 0.19 | 0.0019* |
| 310 | TCST | Whole Pot | 3.4 | 1.8 | 0.018* | 0.56 | 0.25 | 0.00000016* |
| 310 | TCST | Split Pot | 0.67 | 0.50 | 0.77 | 0.23 | 0.20 | 0.41 |
| 311 | TCST | Whole Pot | 1.1 | 0.70 | 0.26 | 0.18 | 0.11 | 0.091* |
| 311 | TCST | Whole Pot | 1.5 | 0.50 | 0.078* | 0.31 | 0.057 | 0.0000010* |
| 311 | TCST | Split Pot | 0.50 | 0.83 | 0.42 | 0.17 | 0.25 | 0.13 |
| 314 | TCST | Split Pot | 0.33 | 0.17 | 1.0 | 0.17 | 0.073 | 0.015* |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. G1073 overexpression via the two-component system resulted in similar phenotypes to those previously observed by us. Namely, many lines exhibited an increase in biomass relative to wild type along with changes in leaf morphology and a slight to moderate delay in flowering time. We have also more extensively examined a number of our original 35S::G1073 direct fusion lines and found that the increased size phenotype is apparent from very early in the rosette stage in some of the lines. The 35S::G1073 seedlings also had more extensively developed roots and longer, denser, root hairs than wild type.

Importantly, the two-component 35S::G1073 lines displayed good tolerance to high salt and sucrose levels during germination compared to the wild type, and also compared to direct 35S::G1073 fusion lines. It is possible that this enhanced performance arose from the two-component approach producing a higher level of G1073 expression than the direct fusion construct. The two-component lines also showed very marked tolerance in soil drought assays. In particular, one line (#311) had very mild morphological changes while exhibiting effective resistance to high salt levels during germination and effective tolerance to drought in a soil assay. Thus, the stress resistance phenotypes obtained with this gene are separable from the developmental changes.

In single pot soil drought assays, three independent 35S::G1073 lines (two direct fusion lines and a two-component line) were examined at well-watered, mild drought, and moderate drought states for a variety of physiological parameters. Apart from an apparent reduction in chlorophyll content in the 35S::G1073 lines versus wild-type at each of the stages, no consistent differences were observed. The physiological basis of the drought tolerance in 35S::G1073 lines is not yet clear and might be related either to parameters that were not measured in the physiology experiments or to changes that were too subtle to detect.

Effects of a full-length G1073 clone versus a short variant. It should be noted that our initial G1073 constructs P448 (35S::G1073, Direct promoter-fusion) and P3369 (opLexA::G1073 for 2-components-supTfa) harbored an N-terminally truncated clone (see sequence details). We have now obtained a full-length G1073 clone (construct P25703) and morphologically examined overexpression lines. 35S::G1073 lines for this full-length cDNA clone showed markedly enhanced biomass in the majority of lines, and no evidence of dwarfing off-types were apparent. In particular, it appears that the increased biomass phenotype is rather more marked and seen at higher penetrance when the full-length clone is overexpressed versus our original truncated clone.

It should be noted that we have obtained similar morphological and physiological phenotypes from overexpression of the related Arabidopsis genes (G2153 and G2156) indicating that these genes are likely to be functionally related.

Potential applications. The results of this study show that G1073 can be used to improve drought related stress tolerance. In light of the different range of effects exhibited by two-component and direct fusion constructs, it may be possible to identify lines with optimal expression patterns for minimized morphological changes with effective stress tolerance. The data also confirm our earlier conclusions that G1073 could be applied to increase biomass and modify flowering time.

G1073 (SEQ ID NO: 113 and 114; Arabidopsis thaliana)—Root ARSK1

Background. The aim of this project was to determine whether expression of G1073 from an ARSK1 promoter, which predominantly drives expression in root tissue, is sufficient to confer stress tolerance that is similar to, or better than, that seen in 35S::G1073 lines. We also wished to test whether the increased biomass seen in 35S::G1073 lines would arise from a root specific expression pattern.

Morphological Observations. Arabidopsis lines in which G1073 was expressed from the ARSK1 promoter (via the two component system) displayed no consistent difference in morphology compared to controls.

Twenty T1 lines were examined (341-360); three lines (#342, 346, 357) were noted to be slightly small and slow developing. However the remainder of the lines exhibited wild-type morphology at all stages. No consistent effects on morphology were noted in the T2 populations that were examined.

Of the lines submitted for physiological assays, all except line 556 showed segregation on selection plates in the T2 generation that was compatible with the transgene being present at a single locus. Lines 556, showed segregation that was compatible with insertions at multiple loci.

Physiology (Plate assays) Results. Seedlings from five ARSK1::G1073 lines had more seedling vigor compared to wild type when germinated on plates containing sodium chloride. Seedlings from two other lines performed better than wild-type in a cold germination assay.

Discussion. We have obtained ARSK1::G1073 lines using a two component approach; no consistent effects on morphology were apparent among these transformants and alterations in leaf size were not observed. Thus, either expression from the ARSK1 promoter was too weak or root expression was not sufficient to trigger the alterations in leaf size that are apparent in 35S::G1073 lines.

Interestingly, although ARSK1::G1073 lines showed no clear morphological changes, five out of ten of these lines did exhibit enhanced tolerance to sodium chloride in a plate based germination assay. Two other lines outperformed wild type in a cold germination assay. These phenotypes are of particular interest, since they show that G1073 can provide stress tolerance independently of changes in organ size. Additionally, since ARSK1 is not significantly expressed in shoot tissue, the results suggest that G1073 expression is not required in the shoot in order to achieve stress tolerance. However, it should be noted that the enhancement of tolerance to salt in plate assays was weaker using the ARSK1 promoter in comparison with the 35S promoter. ARSK1::G1073 lines showed rather inconsistent results in soil drought assays; a single line showed improved tolerance on one plant date, but this was not seen on a second date.

Potential applications. Based on overexpression studies (see 35S::G1073 report), G1073 is a good candidate gene for improvement of abiotic stress tolerance. However, an ARSK1::G1073 combination does not offer significant tolerance in a soil drought assay, in contrast to results with the 35S::G1073 combination under soil drought conditions.

G1073 (SEQ ID NO: 113 and 114; Arabidopsis thaliana)—Epidermal CUT1

Background. The aim of this project was to determine whether expression of G1073 from a CUT1 promoter (which predominantly drives expression in shoot epidermal tissue, with highest levels of expression in guard cells), is sufficient to confer stress tolerance that is similar to, or better than, that seen in 35S::G1073 lines. We also wished to test whether the increased biomass seen in 35S::G1073 lines would arise from an epidermis-specific expression pattern.

Morphological Observations. In Arabidopsis lines that express G1073 from the CUT1 promoter using the two component system, CUT1::LexA; opLexA::G1073, have now been generated. some size variation was apparent at early stages of growth, but overall, the plants showed no consistent differences in morphology to controls.

Plants from each of three CUT1::G1073 T2 populations all showed wild-type morphology.

Physiology (Plate assays) Results. Three CUT1::G1073 lines showed increased seedling vigor when germinated on plates containing sodium chloride. Of these three lines, seedlings of two lines also performed better than wild-type when germinated on sucrose whereas seedlings of the third line had better vigor when germinated on mannitol containing plates. A fourth line showed a better performance only in a sucrose germination assay.

Discussion. We have obtained CUT1::G1073 lines using a two component approach; no consistent effects on morphology were apparent among these transformants and alterations in leaf size were not observed. Thus, either expression from the CUT1 promoter was too weak or epidermal expression was not sufficient to trigger the alterations in leaf size that are apparent in 35S::G1073 lines.

Interestingly, although CUT1::G1073 lines showed no clear morphological changes, three out of ten of these lines did exhibit enhanced tolerance to sodium chloride in a plate based germination assay. Two of these lines also outperformed wild type in a sucrose germination assay, whereas the third line germinated better than wild type on mannitol media. A fourth CUT1::G1073 line gave a positive result in the sucrose assay alone. Although these osmotic stress tolerance phenotypes were seen in a relatively small number of lines, they are of particular interest, since they indicate that G1073 can provide stress tolerance independently of changes in organ size. Additionally, the CUT1 driver line does not give significant expression in the root, suggesting that G1073 expression is not required in the root in order to achieve such tolerance. However, CUT1::G1073 lines did not provide any consistently enhanced tolerance to drought in a soil assay. For example, line 394, which had enhanced tolerance to both salt and sucrose in germination assays, did not confer consistent improvement in a drought assay. Whether this is a function of expression pattern or expression level remains unclear at this time.

Potential applications. Based on overexpression studies (see 35S::G1073 report), G1073 is a good candidate gene for improvement of abiotic stress tolerance. Expression of G1073 using the CUT1 promoter may be inadequate to provide improved performance in a soil drought assay.

G1073 (SEQ ID NO: 113 and 114; Arabidopsis thaliana)—Vascular SUC2

Background. G1073 was included in drought program based on the drought tolerance and enhanced yield shown by 35S::G1073 lines. We have now designated this locus as HERCULES 1 (HRC1).

The aim of this project was to determine whether expression of G1073 from a SUC2 promoter, which predominantly drives expression in a vascular pattern, is sufficient to confer stress tolerance that is similar to, or better than, that seen in 35S::G1073 lines. We also wished to test whether the increased biomass seen in 35S::G1073 lines would arise from a vascular expression pattern.

Morphological Observations. SUC2::G1073 lines exhibited delayed flowering, changes in leaf shape, and increased rosette biomass at late stages of development.

Two component lines. Three sets of 2-component lines have been obtained.

Two sets (#1081-1088, 1101-1105) comprised an opLexA::G1073 construct supertransformed into a SUC2::LexA-GAL4TA promoter driver line (#6). In each of these sets, a number of lines exhibited enlarged leaves and a slight delay in the onset of flowering, as detailed below:

Lines 1081-1088: all appeared wild type at early stages. #1085 and #1088 were slightly late flowering and developed enlarged leaves at later stages. #1082 was also slightly late flowering. The remaining lines showed wild-type morphology at all stages. Lines 1081, 1085 and 1087 were examined in the T2 generation and all showed a wild-type morphology.

Lines 1101-1105: all were slightly small at early stages. #1102 and #1105 were slightly later flowering and #1102 developed enlarged rosette leaves at late stages. The remaining lines all appeared wild type later in development.

The third set of two component lines (#1941-1949) comprised an opLexA::G1073 construct supertransformed into a stronger SUC2::LexA-GAL4TA promoter driver line (#12). These plants showed a markedly stronger phenotype than that seen in the first two sets of lines. At early stages, these plants were small and had narrow leaves with rather long petioles. All the lines showed a marked delay in the onset of flowering and developed much longer curled leaves compared to wild type. It is perhaps worth noting that the morphology of these leaves was somewhat different than in 35S::G1073 lines, where the leaves had a broad round appearance.

Direct promoter-fusion lines. Comparable effects to those seen in the (#1941-1949) 2-component lines were obtained in SUC2::G1073 direct fusion lines. Delayed flowering and enlarged leaves were observed in plants from each of two different sets of lines.

Physiology (Plate assays) Results. Two versions of G1073 under the control of the SUC2 promoter have been used. Both the direct fusion and two-component versions show good tolerance to cold during germination (3/10 two-component lines and 4/10 direct-promoter fusion lines).

Discussion. We have obtained SUC2::G1073 lines using both a two component and direct fusion approach. In each case, a significant number of lines exhibited a delay in the onset of flowering and enlarged leaves relative to controls. This effect became particularly apparent at later developmental stages. Similar phenotypes were obtained at a comparable frequency in 35S::G1073 lines; thus the SUC2 and 35S promoters produced comparable morphological effects when used in combination with G1073. Interestingly, though, the shape of the leaves in SUC2::G1073 and 35S::G1073 plants was subtly different; leaves of the 35S lines were somewhat rounder than in the SUC2 lines. It is not yet clear whether the increase in organ size in the SUC2::G1073 lines arose from G1073 activity within the developing organ primordia themselves or from G1073 protein (or associated signals) moving into shoot meristems or newly incipient primordia from nearby vasculature.

SUC2::G1073 lines have showed improved stress tolerance on plates, particularly to cold treatment. However, results with the soil drought assay have varied, and there is no clear improvement in drought tolerance using the SUC2-promoter.

Potential applications. Based on the effects of overexpression studies (see 35S::G1073 report), G1073 is a good candidate gene for improvement of abiotic stress tolerance. However, the SUC2 promoter does not provide a good option for elimination of morphological effects with retention of drought tolerance. Nevertheless, given the increased biomass seen in these experiments, the SUC::G1073 combination may be used to manipulate traits related to plant organ size.

G1073 (SEQ ID NO: 113 and 114; Arabidopsis thaliana)—Shoot Apical Meristem STM

Background. The aim of this project was to determine whether the efficacy of the G1073 protein in conferring improved abiotic stress tolerance could be maintained by overexpressing the G1073 DNA sequence under the control of a shoot apical meristem-specific promoter.

Morphological Observations. While some lines, similar to 35S::G1073 plants, were late developing, and had broad, serrated leaves, the majority of STM::G1073 lines were similar to wild-type in their development and morphology.

Physiology (Plate assays) Results. Three out of ten STM::G1073 lines were more tolerant to plate-based severe desiccation than controls.

Physiology (Soil Drought-Clay Pot) Summary. One of three lines of STM::G1073 overexpressing plants was more tolerant to drought conditions in a soil-based assay.

G1073 (SEQ ID NO: 113 and 114; Arabidopsis thaliana)—Floral Meristem AP1

Background. The aim of this project was to determine whether the efficacy of the G1073 protein in conferring improved abiotic stress tolerance could be maintained by overexpressing the G1073 DNA sequence under the control of a floral meristem-specific promoter.

Morphological Observations. One line was late developing, but the majority of AP1::G1073 lines examined were similar to wild-type in their development and morphology.

Physiology (Plate assays) Results. Four out of ten AP1::G1073 lines were more tolerant to cold during germination than controls.

G1073 (SEQ ID NO: 113 and 114; Arabidopsis thaliana)—Super Activation (C-GAL4-TA)

Background. The aim of this project was to determine whether the efficacy of the G1073 protein could be improved by addition of a non-native GAL4 activation domain.

Morphological Observations. A total of twelve 35S::G1073-GAL4 T1 lines were isolated. Considerable morphological variation was apparent among the T1 lines. Four of the lines died at early stages. 3/8 (#1550, 1543, 1544) of the surviving plants were small, bushy and showed floral abnormalities. The remaining lines appeared wild type.

Three T2 populations were later examined. Plants from each of these populations appeared wild type.

Physiology (Plate assays) Results. Five out of eight lines containing a GAL4 carboxyl terminal activation domain fusion construct of G1073 were mildly more tolerant to water deficit in a severe plate-based dehydration assay.

Physiology (Soil Drought-Clay Pot) Summary. Two of three 35S::G1073-GAL4 lines tested were significantly more tolerant than controls a soil drought assay, as shown in the table below. A third line had significantly worse survival compared to controls (not shown).

TABLE 67
35S::G1073-GAL4 drought assay results:

| | | | Mean | | | | |
|---|---|---|---|---|---|---|---|
| | | Mean | drought | p-value for | Mean | Mean | |
| | Project | drought | score | drought score | survival for | survival for | p-value for difference |
| Line | Type | score line | control | difference | line | control | in survival |
| 1542 | GAL4 | 3.5 | 0.74 | 0.00036* | 0.56 | 0.19 | 0.000000000065* |
| | C-term | | | | | | |
| 1552 | GAL4 | 1.7 | 0.74 | 0.072* | 0.26 | 0.19 | 0.16 |
| | C-term | | | | | | |

GAL4 C-term = C-terminal translational fusion of transcription factor to a GAL4 acidic activation domain
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have now isolated lines that overexpress a version of the G1073 protein that has a GAL4 activation domain fused to the C terminus. For the most part, lines were indistinguishable from wild type, but some morphological variation was noted in the T1 generation. A few lines with essentially wild-type morphology were tested for enhanced abiotic stress tolerance in plate assays and in soil drought assays. A significant number of the lines showed dehydration tolerance in the plate assays. Two lines (1542 and 1552) exhibited enhanced drought tolerance in soil assays (one of these, line 1552, also exhibiting enhanced growth on germination in mannitol and tolerance to severe dehydration stress in plates).

Potential applications. Based on the data from overexpression studies, G1073 is a good candidate gene for improving stress tolerance in commercial species. Lines with essentially wild-type morphology have showed enhanced drought tolerance, indicating that the C-terminal fusion of the GAL4-TA domain may be used in plants to overcome adverse morphological effects while retaining drought tolerance.

G1073 (SEQ ID NO: 113 and 114; Arabidopsis thaliana)—RNAi (GS)

Background. The aim of this project is to determine whether G1073 is necessary as part of the endogenous protection against drought related stress, by obtaining and testing a knock-down mutant under such conditions. A knock-down mutant for G1073 would assist with genetic analysis to allow a more encompassing understanding of where the gene is positioned in stress tolerance pathways and its mode of action.

Morphological Observations. A number of sets of G1073-RNAi (GS) lines have now been obtained. Overall, these plants showed no consistent differences in morphology to controls. It should be noted, though, that considerable size variation was noted among plants from the first two sets of T1 lines (561-580 and 581-600). However, a final set of lines (1001-1020) showed more uniform growth. Three T2 populations were morphologically examined. Plants from one population (573) were pale and early flowering. This phenotype may have reflected a transgene position effect, and was not observed in the other two lines, which appeared wild type (N.B. P21295 and P21117 are equivalent constructs).

Physiology (Plate assays) Results. Five of ten G1073-RNAi (GS) lines showed a better performance than controls in a severe dehydration assay. Three of these lines also showed a mild tolerance to NaCl in a germination assay. One of the five lines (#573) and another line (which did not show NaCl or dehydration tolerance) also showed tolerance in a heat germination assay. The heat tolerance phenotype was sporadic and was seen in 2/10 lines.

Discussion. We have obtained lines harboring an RNAi construct designed to target only G1073 and not other, related AT-Hook genes. Overall, these lines showed no consistent morphological differences from wild-type.

Seemingly conflicting results were obtained from abiotic stress assays, with lines that had increased salt and severe dehydration tolerance performing more poorly in the soil drought assay. Overexpression of G1073 leads to both increased salt tolerance and drought tolerance, suggesting, perhaps, a more complex relationship than anticipated between salt tolerance and drought tolerance control by G1073-regulated pathways. Focusing on drought tolerance itself, the data are consistent with a normal role for G1073 in drought tolerance. The data also suggest a difference in threshold for morphological and stress tolerance effects.

Potential applications. Based on our previous data, G1073 is a good candidate gene for improvement of stress tolerance and yield. The data from this project suggest that G1073 might play a role in the native pathways that confer tolerance to drought stress.

G1073 (SEQ ID NO: 113 and 114; Arabidopsis thaliana)—RNAi (Clade)

Background. The aim of this project was to further refine our understanding of G1073-regulated pathways by use of an RNAi approach; a construct (see sequence section) was generated that was targeted towards reducing activity of all members of the G1073 clade. Given that the different members of the G1073 clade are potentially functionally redundant, it was thought that this method could reveal phenotypes that might not be visible in single KO lines for the individual clade members.

Morphological Observations. Two sets of G1073-RNAi (clade) lines have been obtained. Overall, no consistent effects on morphology were observed in these plants.

Line Details:

T1 1021-1040: no clear difference to wild type, but 2/20 lines (1023, 1025) showed a slight delay in flowering and developed rather large rosettes compared to controls, at late stages.

T2-1024: all appeared wild type.

T2-1031: 3/6 were small with narrow curled leaves, 3/6 appeared wild type.

T2-1033: all appeared wild type.

T2-1038: all appeared wild type.

T1 1281-1300: no clear differences to controls but 3/20 lines (1283, 1294, 1297 were slightly pale and early flowering).

T2-1284: all appeared wild type.

T2-1296: all appeared wild type.

(N.B. the constructs P21160 and P21301 are identical.)

Physiology (Plate assays) Results. Three out of ten G1073-RNAi (clade) lines showed enhanced tolerance relative to controls in a plate based dehydration assay. One of these lines (#1033) and another line (#1295, which showed a wild-type performance in the dehydration assay) exhibited enhanced NaCl tolerance in a germination assay relative to control plants. In all other respects, G1073-RNAi (clade) lines behaved similarly to wild type in plate assays.

Discussion. We have obtained lines harboring an RNAi clade construct, which is expected to target G2156 and G1073 most effectively, and possibly G1067, G2153 and G1076. These plants appeared morphologically wild type.

Potential applications. A G1073-RNAi (clade) approach may be used to improve stress tolerance and yield.

G1073 (SEQ ID NO: 113 and 114; Arabidopsis thaliana)—Deletion Variant

Background. The aim of this project was to further refine our understanding of G1073 function by use of a “dominant negative” approach in which truncated versions of the protein were overexpressed. Two alternative constructs were built; one of these (P21271) overexpressed a short fragment of the G1073 protein spanning from 8 amino acids before the highly conserved AT-Hook motif to 22 amino acids after that motif. The second construct (P21272) overexpressed a longer G1073 fragment starting at the same position as the short fragment but containing an additional 108 amino acids (for a total of 130 amino acids past the AT-Hook motif), which includes most of the structural domain that is well conserved in G1073-related AT-Hook proteins.

Morphological Observations. Lines have been obtained for each of two different G1073 dominant negative constructs: P21271 and P21272. These constructs were each designed to overexpress a highly conserved central portion of the G1073 protein. P21271 contained a shorter fragment of G1073 than P21272 (see sequence notes).

Lines 861-880 were transformed with P21271. The majority of these lines displayed a wild-type phenotype and appeared wild type at all developmental stages. However, 4/20 lines (872, 879, 880, 870) were noted to have marginally larger leaves than wild type at the end of the rosette stage. This phenotype was subtle and was not observed at other developmental stages.

Line sets 881-889 and 1261-1280 each contained the construct P21272. Eight out of these twenty-nine plants (883, 886, 888, 889, 1264, 1265, 1270, and 1278) showed a variety of severe developmental defects and were extremely dwarfed, poorly fertile and often had contorted leaves. A number of the dwarf plants were also early flowering compared to controls. The remaining lines in the P21272 sets exhibited wild-type morphology.

Three T2 lines for P21272 were examined (see table below); plants from these populations showed wild-type morphology.

Discussion. We have now isolated lines overexpressing each of the G1073 deletion variant constructs. In contrast to overexpression lines for the full-length version of the gene, these truncated versions did not produce any improved tolerance to abiotic stress in plate assays or the soil drought assay. The majority of lines for each of the constructs exhibited wild-type morphology; however, in each case, a significant number of plants displayed developmental alterations.

Approximately 25% of the overexpression lines containing the longer peptide (P21272) exhibited deleterious morphological effects (dwarfing and contorted leaves). Such effects are not readily apparent among regular 35S::G1073 lines. However, it should be noted that such effects were frequently seen among overexpression lines for the related genes G2153 and G2156. Thus, it seems that sequences outside of the conserved domain (which was present in the longer deletion variant) are the responsible for the differences in dwarfing phenotypes seen with the paralogs versus G1073 itself.

Potential applications: Based on the data from overexpression studies, G1073 and the related proteins are good candidates for improving stress tolerance in commercial plant species. The results from these deletion variants demonstrate that the conserved AT-hook region of G1073 is not by itself sufficient to produce increased size and stress tolerance.

G1073 (SEQ ID NO: 113 and 114; Arabidopsis thaliana)—Double Overexpression

Background. The aim of this double overexpression approach is to determine whether different transcription factors will give an additive effect on drought tolerance when “stacked” together in the same line. The combinations (3)-(6) below are being made to test whether the delayed flowering produced by overexpression of G1073 can be overcome by a transgene that accelerates flowering.

Morphological Observations. Crosses have been set up between 35S::G1073 and the other overexpressing lines as follows:

(1) 35S::G1073 Line 4×35S::G481 (SEQ ID NO: 1) Line 3

A double homozygous line for this combination has been constructed and showed an additive morphological phenotype between G1073 and G481 overexpression (see “G481 (SEQ ID NO: 1 and 2; Arabidopsis thialiana)—Double Overexpression”, results for (1) 35S::G481×35S::G1073 (SEQ ID NO: 113)”, presented above.

(2) 35S::G1073 Line 3×35S::G682 (SEQ ID NO: 59) Line 16

This cross has been set-up and seed from an F2 population is in hand. We are currently in the process of screening for a double homozygous line. The double overexpressing individuals in the F1 and F2 populations showed an additive phenotype between G1073 and G682 overexpression; these plants were small at early stages, showed a glabrous phenotype, were late flowering, and developed broad, slightly serrated leaves. Overall, the size-related aspect of the phenotype was comparable to that seen in 35S::G1073 lines; overexpression of G1073 effectively overcame the dwarfing that is associated with G682 overexpression.

(3) 35S::G1073 Line 4 (Male)×35S::G3086 (SEQ ID NO: 291) Line 8 (Female)

Eleven F1 plants were obtained. All F1 plants showed an intermediate leaf phenotype, but were earlier flowering than wild-type. Thus, the accelerated flowering produced by G3086 overexpression appears epistatic to the delayed flowering associated with G1073 overexpression. The double overexpressing plants had broader leaves than the 35S::G3086 parental line, but the leaves were smaller than in wild-type and the 35S::G1073 parental line.

(4) 35S::G1073 Line 4×35S::G867 (SEQ ID NO: 87) Line 8

Seeds from an F1 population are currently in hand, but a double homozygote has not yet been identified. The F1 lines showed no consistent differences to wild-type and exhibited neither the large leaves characteristic of G1073 overexpression nor the dwarfing that is characteristic of G867 overexpression lines.

(5) 35S::G1073 Line 4×35S::G1274 (SEQ ID NO: 185) Line 16

Twelve F1 plants were obtained and these showed a strikingly additive morphological phenotype. The leaves of these plants were even larger than in either the 35S::G1073 or the 35S::G1274 parental line. The double overexpression line, however, still showed the loss of apical dominance that is characteristic of G1274 overexpression.

Physiology (Soil Drought-Clay Pot) Summary. One independent 35S::G1073 line 4×35S::G481 line 3 line was tested, and this line exhibited significantly better survival relative to controls.

TABLE 68
35S::G1073 line X 35S::G481 drought assay results:

| | | Mean | Mean | p-value for | Mean | Mean | p-value for |
|---|---|---|---|---|---|---|---|
| | | drought | drought | drought score | survival for | survival | difference in |
| Line | Project Type | score line | score control | difference | line | for control | survival |
| F1-1- | Double OEX | 3.4 | 2.3 | 0.021* | 0.57 | 0.36 | 0.00070* |
| F1-1- | Double OEX | 2.4 | 1.8 | 0.10* | 0.57 | 0.31 | 0.000011* |

OEX = Double overexpression
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion A crossing strategy was initiated to construct the above lines; details of progress are shown in the above “Morphological Observations”. Double overexpressors have been obtained for the 35S::G1073;35S::G682 combination, but we are still in the process of obtaining a double homozygote. However, these lines showed an intermediate morphology between those of the parental lines, having broad glabrous leaves. Overall, the size-related aspect of the phenotype was comparable to that seen in 35S::G1073 lines; overexpression of G1073 effectively overcame the dwarfing that is associated with G682 overexpression. One line, # F1-1-46, a 35S::G1073 line×35S::G481 double overexpressor line, showed greater drought tolerance than controls in repeat assays.

We have also obtained F1 plants from cross (3); these flowered earlier than wild-type, demonstrating that overexpression of G3086 can overcome the delay in flowering that results from G1073 overexpression. The leaves of the double overexpression line were broader and rounder than those of the 35S::G3086 parental line, but were smaller than those of wild-type and the 35S::G1073 parental line.

F1 plants were obtained from cross (9) and these showed a strikingly additive morphological phenotype. The leaves of these plants were even larger than in either the 35S::G1073 or the 35S::G1274 parental line. The double overexpression line, however, still showed the loss of apical dominance that is characteristic of G1274 overexpression.

Potential applications. Both the 35S::G3086;35S::G1073 and the 35S::G1073;35S::G1274 combinations indicate that a stacking approach might offer advantages over either of the 35S::G1073 transgene alone.

For example, 35S::G1073 in soybean produces a delayed flowering off-type which is associated with a yield penalty (field trial data). Combining G1073 and G3086 overexpression in the same line may afford drought tolerance without the delayed flowering caused by G1073.

A combination of 35S::G1274 and 35S::G1073 can be of particular value for applications where an increase in vegetative biomass is desired, such as in green leafy vegetables or in forestry crops. Additionally, since both G1073 and G1274 give very good drought tolerance when used singly, it is expected combining the two transgenes might give an exceptional tolerance to drought. It should also be noted that 35S::G1073 soybean lines show a strongly increased apical dominance off-type which leads to a yield reduction (field trial data). We have shown that the 35S::G1274;35S::G1073 double exhibits a reduced apical dominance phenotype in Arabidopsis, indicating that combining 35S::G1274 with 35S::G1073 in soybean will eliminate the off-type which is associated with the latter.

G1067 (SEQ ID NO: 119 and 120; Arabidopsis thaliana)—Constitutive 35S

Background. G1067 is a paralog of G1073. This gene and G2156 are the most related Arabidopsis paralogs of G1073, according to phylogenetic analysis.

G1067 corresponds to ESCAROLA (ESC) and the morphological effects of its overexpression have been documented by Weigel et al. (2000); these included slow growth, delayed flowering and leaf curling. Such observations were confirmed during our earlier genomics program.

The aim of the current study was to re-evaluate the effects of G1067 overexpression using a two-component approach.

Morphological Observations. We have so far been able to recover a total of only five 35S::G1067 (2-component) lines despite making selection attempts on six aliquots of T0 seed that were derived from three independent transformations.

The paucity of transformants recovered suggests that G1067 might have lethal effects when overexpressed via the two component system. Previously, 35S::G1067 direct promoter fusion lines were found to exhibit a variety of deleterious phenotypes. It is possible that a higher level of G1067 activity was attained with a two component approach and that this impeded the isolation of transformants.

Of the two-component lines that were obtained, four (#301, 302, 441, 442) of the five T1 lines were smaller and slow developing compared to controls. The final line #303 was tiny and arrested growth early in development.

Three lines (#301, 302, 442) examined in the T2 generation showed no consistent differences in morphology compared to controls, suggesting that the transgenes might have been less active than in the T1 generation.

An attempt was made to isolate additional 35S::G1067 direct fusion lines (these were shown to be dwarfed and have curled leaves), but no transformants were recovered.

Physiology (Plate assays) Results. Due to the deleterious effects of G1067 overexpression, only four 35S::G1067 lines were available for plate assays. Two (#441 and #442) of these four lines showed a better performance than controls on ABA germination plates. Line 441 also performed better than wild type in a cold growth assay.

Physiology (Soil Drought-Clay Pot) Summary. One of three independent 35S::G1067 (2-component) lines tested (#442) exhibited significantly better survival relative to controls.

TABLE 69
35S::G1067 (drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 442 | TCST | 3.0 | 0.89 | 0.024* | 0.31 | 0.19 | 0.049* |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Overexpression lines have been obtained using the two-component expression system. Lines generally exhibited an exacerbated phenotype compared to the phenotype observed in our earlier genomics program, and were very small and slow growing. No obvious increases in leaf size were noted, as were seen with G1073 overexpression. It should also be noted that two-component lines were obtained at very low frequency, possibly indicating that high level overexpression produced lethality. Four of the five lines produced T2 progeny with relatively wild-type morphology. The T2 progeny of one of these lines, #442, was more tolerant to drought treatment than the wild type in a clay pot assay, and was also more tolerant to ABA than wild type in a germination assay. A second line was both more tolerant to ABA in a germination assay and more tolerant to cold in a growth assay.

Potential applications. G1067 has been shown to confer improved tolerance to drought-related stress, although G1067 was fairly toxic to most transformants. It is possible that specific expression patterns or levels are necessary to enable recovery of wild type plants with enhanced drought tolerance. Interestingly, one of the 35S::G1067 lines (#442) from our studies could be an example of such an instance, since we obtained stress tolerance without any obvious morphological changes.

G1067 (SEQ ID NO: 119 and 120; Arabidopsis thaliana)—Stress Inducible RD29A—Line 5

Background. The aim of this project was to determine whether expression of G1067 from a stress inducible promoter (RD29A) would confer enhanced tolerance to drought related stress.

A two component approach was used for these studies and two different RD29A::LexA promoter driver lines were established: line 2 and line 5. Line 2 had a higher level of background expression than line 5, and thereby is expected to provide somewhat different regulation. Line 2 was observed to have constitutive basal expression of GFP, and to have a marked increase in GFP expression following the onset of stress. In contrast, line 5 exhibited very low background expression, although it still exhibited an up-regulation of expression following the onset of stress. However, the stress-induced levels of GFP expression observed in line 5 were lower than those observed for line 2.

Morphological Observations. Supertransformants for opLexA::G1067 into the RD29A_line5::LexA promoter background were obtained. Two sets of lines were isolated (661-670; 701-720). Approximately 50% of the lines showed changes in leaf size and shape and 50% of the lines appeared wild type.

Line Details:

T1 Lines 661-670: 7/10 lines (#661, 662, 664, 667, 668, 669, 770) were slightly small, with rather rounded leaves, at the rosette stage, but later appeared wild type. The remaining plants appeared wild type throughout development.

T1 Lines 701-720: #714, 716, 718 were small with rounded leaves at early stages. The remaining lines appeared wild type, early on. At later stages 8/20 lines, (#702, 703, 705, 709, 710, 711, 715, 720) displayed short, wide rosette leaves that curled downwards at the margins. Other lines from this set appeared wild type.

T2-664: 4/6 showed broad curled leaves and enlarged rosettes at later stages.

T2-710: 2/6 showed slightly broad leaves.

Physiology (Plate assays) Results. Six out of the ten lines showed good tolerance to dehydration stress in a severe dehydration assay. In addition, some lines also performed well in germination assays on mannitol, sucrose, ABA, cold, and sodium chloride (lines 711, 717, 708, and 710). Lines 707 and 708 had an increased root hair phenotype.

Physiology (Soil Drought-Clay Pot) Summary. Drought experiments on supertransformants for opLexA::G1067 into the RD29A_line5::LexA promoter background indicate that this gene/promoter combination might offer an advantage under drought conditions.

Three independent lines were initially tested in a split pot soil drought assay (line and controls together in same pot). One line (#404) showed less severe stress symptoms at the end of dry-down compared to controls.

Three different lines were later tested in whole pot soil drought assays. Two of these three lines (711 and 717) each showed a better survival than wild type on one of two plant dates. The survival of each of those lines was not statistically distinct from controls on the second plant date.

TABLE 70
RD29A::G1067 drought assay results:

| | | | Mean | Mean | p-value for | Mean | Mean | p-value for |
|---|---|---|---|---|---|---|---|---|
| | Project | | drought | drought score | drought score | survival | survival for | difference |
| Line | Type | Assay type | score line | control | difference | for line | control | in survival |
| 711 | TCST | Whole Pot | 1.8 | 1.6 | 0.94 | 0.42 | 0.25 | 0.0037* |
| 711 | TCST | Whole Pot | 2.2 | 1.2 | 0.072* | 0.34 | 0.30 | 0.44 |
| 704 | TCST | Split Pot | 0.75 | 0.17 | 0.011* | 0.13 | 0.071 | 0.12 |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have now established two component (RD29A::LexA;opLexA::G1067) lines in the RD29A line 5 background. The majority of these lines showed no consistent alterations in morphology relative to controls. However, some of the transformants did show a small reduction in size and slightly more rounded leaves than controls. At later stages, in some of the second generation (T2) lines, enlarged leaves and slightly delayed flowering were seen, indicating that G1067 can produce similar effects on plant morphology to G1073. In these lines, low constitutive expression produced by the driver line could have triggered such effects. However, it should be noted that none of the lines showed the extreme dwarfing and curled leaves seen in 35S::G1067 lines.

In plate assays, several lines were more tolerant to severe dehydration, mannitol and sodium chloride. Lines 704 and 711, which generally performed better than wild type in plate stress assays, also performed better than wild type in a soil drought treatment.

Potential applications. Drought-inducible expression of G1067 can provide improved drought tolerance, without deleterious effects on plant morphology as are seen in 35S::G1067 lines.

G1067 (SEQ ID NO: 119 and 120; Arabidopsis thaliana)—Leaf RBCS3

Background. The aim of this project was to determine whether expression of G1067 from an RBCS3 promoter, which predominantly drives expression in photosynthetic tissue, would eliminate or alleviate the morphological effects of overexpression. We also wished to test whether RBCS3::G1067 lines show enhanced tolerance to abiotic stress.

Morphological Observations. Arabidopsis lines in which G1067 was expressed from the RBCS3 promoter (using the two component system) exhibited changes in size, altered leaf shape and in some cases showed slightly delayed flowering, as detailed below.

Line Details:

T1 Lines 581-590: all were small, particularly at early stages and exhibited rather rounded short leaves. At later stages, the leaves often became contorted and curled. All lines showed a slight delay in the onset of flowering (about 1-5 days under 24-hour light).

T1 Lines 621-629: all were slightly small at early stages and had short, round, rather broad leaves. Delayed flowering was not noted in this set of lines.

T2-587: no consistent differences to wild-type. (on one plant date, this line was slightly early flowering, but the effect was not seen on other dates).

T2-588: slightly delayed flowering and slightly enlarged curled leaves (this phenotype was only recorded in one of three plantings and could be conditional).

T2-627: no consistent differences to wild-type. (on one plant date, this line was slightly early flowering, but the effect was not seen on other dates).

Physiology (Plate assays) Results. Four out of the 10 RBCS3::G1067 lines showed good performance when germinated on plates containing sodium chloride.

Discussion. We have obtained RBCS3::G1067 lines using a two component approach; these plants were generally small at early stages, had short rounded leaves and flowered slightly late. At later stages of growth, the leaves became contorted and curled, but in occasional lines leaves were broader than those of controls. The appearance of broad leaves, albeit at a low frequency, indicates that G1073 and G1067 might, at least to some extent, be functionally related. In comparison with constitutive G1067 expression, expression from the leaf RBCS3 promoter gave much attenuated morphological effects. Three lines with relatively wild-type morphology yielded improved growth in a NaCl germination assay. However, the results from soil drought assays were rather inconsistent. A single line did show increased tolerance in one run of the experiment, but in a different planting, showed a comparable result to wild-type.

Potential applications. The utility of the RBCS3::G1067 combination with respect to drought tolerance remains unclear. However, based on the morphological effects seen in these lines, the combination may be used to modify flowering time or leaf shape. Importantly, the increased leaf size seen in these lines demonstrates that the capacity to effect this phenotype is shared by G1073-related proteins and is not specific to G1073 itself.

G1067 (SEQ ID NO: 119 and 120; Arabidopsis thaliana)—Root ARSK1

Background. The aim of this project was to determine whether expression of G1067 from an ARSK1 promoter, which predominantly drives expression in root tissue, would eliminate or alleviate the detrimental morphological effects of constitutive overexpression, and whether ARSK1::G1067 lines show enhanced tolerance to abiotic stress.

Morphological Observations. The majority of Arabidopsis lines in which G1067 was expressed from the ARSK1 promoter (via the two component system) displayed no consistent difference in morphology compared to controls. However, a number of the lines showed a reduction in overall size and developed slowly relative to controls.

T1 Lines Details:

Lines 341-347: 3/7 (#342, 343, 346) lines were very small, 4/7 lines appeared wild type.

Lines 401-409: 2/9 (#403, 404) lines were small, 7/9 appeared wild type.

Lines 481-490: 3/10 (#483, 488, 489) lines were very small, 7/10 appeared wild type.

T2 line details:

T2-345: all plants appeared wild type.

T2-346: 2/6 were tiny, 3/6 were slightly small, 1/6 was wild type.

T2-347: all plants were small at early stages and developed more slowly than wild type.

T2-401, T2-402, and T2-406; plants showed no consistent differences to wild-type controls.

Of the lines submitted for physiological assays, all lines showed segregation on selection plates in the T2 generation that was compatible with the transgene being present at a single locus.

Physiology (Plate assays) Results. Four out of ten lines were more resistant than wild-type seedlings in a severe dehydration assay. Interestingly, some lines that were not tolerant to dehydration stress had better seedling vigor when germinated on plates containing salt compared to wild-type seedlings.

Discussion. We have obtained ARSK1::G1067 lines using a two-component approach; the majority (18 out of 26) of these transformants appeared wild type, and displayed no evidence of curled leaves or severe dwarfing. However, 8 of 26 lines showed size reductions and developed more slowly than controls, to various extents.

ARSK1::G1067 lines have now been tested in plate based stress assays, and four out often lines examined showed enhanced tolerance in a severe dehydration assay. All of these four lines had shown a wild-type phenotype in the morphological screens, demonstrating that G1067 could enhance dehydration stress tolerance without producing obvious negative effects on plant size. Four other ARSK1::G1067 lines showed a wild-type response in the dehydration assay but were more tolerant than controls in an NaCl germination assay.

Potential applications: Based on the above results, G1067 might be applied to improve tolerance to drought related stress in commercial species. Given the undesirable morphologies that arise from G1067 overexpression via a constitutive promoter, the gene might be optimized for product development by its combination with this or another root-specific promoter.

G2153 (SEQ ID NO: 137 and 138; Arabidopsis thaliana)—Constitutive 35S

Background. G2153 was included in drought program based on its sequence relatedness to G1073 and its tolerance to osmotic stress in our earlier genomics screens. This gene and G1069 are the most related Arabidopsis homologs of G1073, according to phylogenetic analysis. There have been no data documenting functional characteristics of G2153 in the public domain.

Morphological Observations. Additional sets of G2153 overexpression lines have now been obtained by both the two component system (P6506, P4524) and a 35S direct promoter fusion construct (P1740).

The majority of plants produced via each of the two methods exhibited marked alterations in leaf shape; petioles were short and leaf blades were rounded and had a ruffled, curled, appearance compared to wild type. The plants were also generally small, slow developing, flowered much later than controls, and yielded relatively few seeds. These latter results were somewhat comparable to those obtained during our earlier genomics studies, when a considerable number of the 35S::G2153 lines were observed to be small and occasionally to show leaf curling. However, two component lines typically showed the stronger phenotypes, perhaps suggesting that higher levels of G2153 activity were attainable using that system compared to a direct promoter fusion approach.

In addition to the above effects on leaf shape and size, a minor percentage of the lines showed a phenotype not observed during our earlier genomics study. At early stages, plants possessing this phenotype appeared either wild type, or reduced in size, but at later stages, their lateral organs continued to grow and expand for longer than in wild-type, resulting in leaves and floral organs (particularly petals) becoming markedly enlarged. This phenotype was generally more apparent in the set of direct fusion lines than the two component lines. Details of lines and phenotypes are given below:

Direct promoter fusion lines: Lines 341-354: considerable size variation was seen at the vegetative stage with #342, 343, 345, 347, 351, and 354 being particularly small with abnormal shaped leaves. At this stage, the remaining plants were slightly small, or wild type. All plants in the set flowered later than wild-type to varying extents. Four of the fourteen lines (#345, 349, 350, 354) showed enlarged organs, and of these, #350 had such effects apparent at the early inflorescence stage, whereas other lines showed the phenotype only towards the end of the life cycle. Line 345 also produced a particularly large quantity of seed.

(Of the lines submitted for physiological assays, the following had segregation on selection plates in the T2 generation that was compatible with the transgene being present at a single locus: 342, 343, 345, 347, 349, 354. Lines 341, 348, 350, 352 showed segregation that was compatible with insertions at multiple loci.)

The following lines were examined in the T2 generation:

T2-352: all small at early stages, late flowering, has rather curled leaves (at margins) and increased rosette biomass at late stages.

T2-348: 4/6 showed broad leaves at late rosette stage. All were later flowering than control. Some evidence of leaf curling was apparent.

T2-341: 4/6 showed a mild delay in the onset of flowering, but otherwise, no consistent alterations in morphology were apparent.

Two component lines: Lines 301-308: all were very small, slow developing, with curled ruffled leaves. None of these lines developed increased biomass versus wild type.

Lines 321-324: all were very small, slow developing, with curled ruffled leaves. None of these lines developed increased biomass versus wild type.

Lines 361-365: all were very small, slow developing, with curled ruffled leaves. #362, 365 exhibited enlarged rosettes at late stages.

Lines 381-383: all were very small, slow developing, with curled leaves.

Lines 401-411: all were very small, slow developing, and showed curled ruffled leaves. Line 401 exhibited increased biomass at late stages.

The majority of two component lines displayed severe size reductions and produced few if any seeds.

Three lines were examined in the T2 generation: T2-401, T2-406, T2-411. In each case, the plants were tiny, had curled leaves, and were very late flowering. Some of these plants died early on, but those that survived recovered at late stages of growth and developed large rosettes relative to controls.

Flowers from a number of 35S::G2153 (direct promoter-fusion) lines were larger than flowers from wild type.

Physiology (Plate assays) Results. Overexpression of G2153 in Arabidopsis resulted in seedlings with an altered response to osmotic stress. In a germination assay on media containing high sucrose, G2153 overexpressors had more expanded cotyledons and longer roots than the wild-type controls.

We have now re-examined a larger number of direct promoter-fusion 35S::G2153 lines. Nine of ten new lines tested outperformed wild type to varying extents in a number of different plate based assays. These included decreased tolerance to NaCl, sucrose, ABA, germination in cold and growth in cold, as well as reduced sensitivity to ABA, relative to wild type. However, seven of the 35S::G2153 lines were rather small and had little root growth.

Similar phenotypes were obtained with two-component lines and comparable results were seen in the same assays as for the direct fusion lines.

Physiology (Soil Drought-Clay Pot) Summary.

Direct promoter fusion lines: 35S::G2153 direct promoter fusion lines showed strongly enhanced survival relative to wild-type in soil drought assays. Four independent lines were tested and three of those lines showed a consistently better rate of survival than wild-type controls. Line 352 showed particularly strong tolerance and exhibited significantly better survival than wild-type on each of the three plant dates on which it was tested. Line 348 exhibited significantly better survival than wild-type on each of the two plant dates when it was tested. Line 343 showed better survival on the one plant date on which it was tested.

TABLE 71
35S::G2153 drought assay results

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 343 | DPF | 5.3 | 2.6 | 0.0015* | 0.48 | 0.25 | 0.000058* |
| 348 | DPF | 0.50 | 0.10 | 0.13 | 0.093 | 0.021 | 0.019* |
| 348 | DPF | 1.0 | 0.60 | 0.27 | 0.21 | 0.086 | 0.0035* |
| 352 | DPF | 4.0 | 2.6 | 0.13 | 0.55 | 0.25 | 0.00000037* |
| 352 | DPF | 2.1 | 0.10 | 0.0055* | 0.39 | 0.021 | 0.000000063* |
| 352 | DPF | 1.9 | 0.10 | 0.0018* | 0.24 | 0.014 | 0.000025* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have generated lines for both direct fusion and two component constructs. Lines from the two approaches exhibited similar effects. The majority of transformants were small, slow developing and had abnormally shaped leaves. However, a significant proportion of the lines developed enlarged lateral organs (leaves and flowers), particularly at later developmental stages. It should be noted that a greater frequency of deleterious phenotypes was seen among the two-component lines. Since other data indicate that two-component lines have increased expression levels in comparison with direct fusion constructs, this suggests that deleterious morphological effects are dose-dependent.

Abiotic stress assays have now been performed on a set of the direct fusion and two-component lines. These experiments confirmed our earlier observations that 35S::G2153 lines have enhanced tolerance to abiotic stress. In our latest studies, positive phenotypes were seen in NaCl, sucrose, ABA, and cold stress assays. Three different direct fusion lines also showed a strong performance relative to controls in “whole-pot” soil drought assays. Interestingly, three different two-component lines tested in “split-pot” soil drought assays all performed worse than wild-type, perhaps due to the competition between these slow-growing lines and controls in this format.

It is particularly interesting that effects similar to those exhibited by 35S::G2153 lines on organ growth and stress tolerance have been obtained with 35S::G1073 and 35S::G2156 lines, indicating that these genes are functionally related.

Potential applications. Based on the results of our overexpression studies on G2153 and related genes, G2153 is a potential candidate for improvement of drought related stress tolerance in commercial species. Although this work provides further evidence for improved drought tolerance conditioned by members of the AT-Hook family, stress tolerant G2153 lines also had a dramatic flowering delay and morphological effects.

Based on the developmental effects observed, G2153 could be used to manipulate organ growth and flowering time.

G2156 (SEQ ID NO: 129 and 130; Arabidopsis thaliana)—Constitutive 35S

Background. G2156 was included in the drought program as a potential paralog to G1073. Based on amino acid sequence, the G2156 protein along with G1067 is phylogenetically more closely related to G1073 than the other members of the study group. There have been no data documenting functional characteristics of G2156 in the public domain.

Our earlier genomics screen characterized 35S::G2156 lines as having multiple morphological alterations, but did not reveal any enhancement of abiotic stress tolerance. The aim of this study was to re-examine the effects of G2156 overexpression, particularly with respect to abiotic stress responses.

Morphological Observations. Additional sets of G2156 overexpression lines have now been obtained by both the two component system (P6506, P4418) and a 35S direct promoter fusion construct (P1721).

The majority of plants produced via each of the two methods exhibited marked alterations in leaf shape; petioles were short and leaf blades were rounded and had a ruffled, curled appearance. The plants were also generally very small, slow developing, flowered later than controls, and yielded relatively few seeds. Occasional lines, though, showed early flowering. These results were comparable to those obtained during our earlier genomics studies. However, two component lines typically showed the stronger phenotypes, perhaps suggesting that higher levels of G2156 activity were attainable using that system compared to a direct promoter fusion approach.

In addition to the above effects on leaf shape and size, a minor percentage of the lines showed a phenotype not observed during our earlier genomics study. At early stages, plants possessing this phenotype appeared either wild type, or reduced in size, but at later stages, their lateral organs continued to grow and expand for longer than in wild-type, resulting in leaves and floral organs particularly petals) becoming markedly enlarged. This phenotype was generally more apparent in the set of direct fusion lines than the two component lines.

Line Details:

Direct promoter fusion lines. T1 lines 421-440: considerable size variation was seen at the vegetative stage with #423, 424, 425, 426, and 427 being particularly small with abnormal shaped leaves. At this stage, the remaining plants were slightly small, or wild type. Many of the plants in the set flowered later than wild type to varying extents, but two lines, T1-433 and T1-439 were somewhat early flowering. Five of the fourteen lines (#421, 428, 436, 437, 438) showed enlarged organs. Unfortunately (due to a lab mishap) seed from lines #436, 437, 438 were not obtained.

T2-421: all showed broad curled leaves, were small at early stages, and flowered late.

T2-423: all very small and pale at early stages, showed highly curled leaves and flowered late.

T2-424: all slightly small at early stages with mild leaf curling. Flowering was slightly delayed and leaves were slightly enlarged at late stages.

T2-425: all showed accelerated flowering and were pale in coloration.

(Of the lines tested in physiological assays, the following had a segregation on selection plates in the T2 generation that was compatible with the transgene being present at a single locus: 424, 425, 435. Lines 421, 422, 428, 429, 434, 432 and 431 showed segregation that was compatible with insertions at multiple loci.)

Two component lines. T1 lines 301-307: all were tiny with curled leaves and died at early stages.

T1 lines 321-326: all were tiny with curled leaves, and slow developing. #321, 325, 326 died at early stages

T1 lines 381: only a single plant was obtained in this set, which was tiny, showed curled leaves and developed very slowly.

T1 lines 401-403: #402 and 403 were tiny and late flowering. #401 appeared wild type at early stages, but later developed enlarged leaves and flowers.

Since the majority of two component lines displayed severe size reductions and produced few if any seeds, lines were not available for soil drought assays.

Physiology (Plate assays) Results. 35S::G2156 seedlings had previously displayed a wild-type response in physiological assays. We have now examined a greater number of lines.

Eight out of ten new 35S::G2156 direct fusion lines showed a good performance versus wild-type when germinated on plates containing sodium chloride. In addition, one line (425) had more vigor when analyzed on both heat germination and cold growth plates. Another line (421) performed better than wild-type on a germination assay on plates containing sucrose.

Three 35S::G2156 two-component lines were also tested and tolerance was noted in sucrose and ABA germination assays and in a cold growth assay.

Physiology (Soil Drought-Clay Pot) Summary. Three independent 35S::G2156 (direct fusion) lines have been tested in soil drought assays. Two of these lines showed a significantly better performance than wild-type on one of two dates on which a “whole pot” assay with moderate drought conditions was run. Both of the lines showed a comparable performance to wild type on a second plant date. However, it should be noted that on the second date, the plants suffered a somewhat harsher treatment. Thus, this gene might confer an advantage under moderate but not severe drought conditions.

TABLE 72
35S::G2156 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 421 | DPF | 0.10 | 0.10 | 1.0 | 0.036 | 0.046 | 0.75 |

Discussion. We have generated lines using both direct fusion and two component constructs. Lines from the two approaches exhibited similar effects. The majority of transformants were small, slow developing and had abnormally shaped leaves. However, a significant proportion of the lines developed enlarged lateral organs (leaves and flowers), particularly at later developmental stages. It should be noted that a greater frequency of deleterious phenotypes was seen among the two-component lines. Since other data indicate that two-component lines have increased expression levels in comparison with direct fusion constructs, this suggests that deleterious morphological effects are dose-dependent.

Plate-based physiology assays revealed an effect not observed during our earlier studies: 35S::G2156 lines showed enhanced tolerance in a germination assay on sodium chloride media. An enhanced performance was also seen in ABA, sucrose, and chilling growth assays. Three lines (#421, 424 and 425) were tested in soil drought assays, with the former two lines performing better than controls under mild drought stress, while the latter line performed worse than controls under both mild and more severe drought treatment. In contrast to lines 421 and 424 which flowered late and had curled leaves, line 425 flowered early and had pale leaves.

It is particularly interesting that effects similar to those exhibited by 35S::G2156 lines on organ growth and stress tolerance have been obtained with 35S::G1073 and 35S::G2153 lines, indicating that these genes are functionally related.

Potential applications. Based on the results of our overexpression studies on G2156 and related genes, G2156 is a potential candidate for improvement of drought related stress tolerance in commercial species. Although this work provides further evidence for improved drought tolerance conditioned by members of the AT-Hook family, stress-tolerant lines also had delayed flowering and altered morphology.

Based on the developmental effects observed, G2156 could be used to manipulate organ growth and flowering time.

G2156 (SEQ ID NO: 129 and 130; Arabidopsis thaliana)—Root ARSK1

Background. Many of the 35S::G2156 lines have previously been shown to have marked reductions in overall size and a range of undesirable effects such as slow growth, curled leaves, altered coloration, and poor fertility. In our original genomics experiments, overexpression lines with very severe morphological changes could not be tested in stress assays. The aim of this project was to determine whether expression of G2156 from an ARSK1 promoter, which predominantly drives expression in root tissue, would eliminate or alleviate the undesirable morphological effects of overexpression. We also wished to test whether G2156, when combined with an ARSK1 promoter, would result in increased abiotic stress tolerance.

Morphological Observations. The majority of Arabidopsis lines in which G2156 was expressed from the ARSK1 promoter (via the two component system) displayed no consistent difference in morphology compared to controls.

A total of 60 T1 lines from three different batches were examined, as detailed below:

Lines 361-380: 5/20 (#361, 362, 366, 367, 364) lines were small, 15/20 appeared wild type.

Lines 481-500: all showed wild-type morphology, but 12/20 (#483, 484, 485, 486, 487, 488, 489, 490, 493, 495, 499, 500) displayed a marginal delay in the onset of flowering (this effect did not recapitulate when three of those lines were examined in the T2 generation (see below).

Lines 601-620: all appeared wild type.

Six different T2 populations were also morphologically examined:

T2-364: all plants were wild type.

T2-365: all appeared wild type.

T2-368: 3/6 were small, 3/6 were slow developing.

T2-484: all appeared wild type.

T2-485: all appeared wild type.

T2-489: all appeared wild type.

T2-488: all appeared wild type.

T2-494: all appeared wild type.

Of the lines submitted for physiological assays, all showed a segregation on selection plates in the T2 generation that was compatible with the transgene being present at a single locus.

Physiology (Plate assays) Results. Four out of ten ARSK1::G2156 lines performed better than wild-type in a severe plate based drought assay.

Discussion. We have obtained ARSK1::G2156 lines using a two component approach; three independent batches of transformants were obtained. Approximately half of the lines from one of these batches displayed a very marginal delay in the onset of flowering, but the majority of lines displayed no obvious differences in growth and development to wild-type controls. Thus, use of a root promoter in combination with G2156 largely eliminated the undesirable morphologies produced by overexpression of that gene with the 35S promoter.

ARSK1::G2156 lines were tested in plate based assays; four out often lines performed better than controls in a severe dehydration assay. This is of particular interest since a comparable result was obtained in the ARSK1 experiment for the closely related gene, G1067, highlighting the fact that these two genes likely have comparable functions.

The above dehydration assay results indicate that an increase in G2156 activity can confer abiotic stress tolerance. However, two of three ARSK1::G2156 lines tested performed more poorly than controls in a soil drought assay. These results a more complex relationship than expected between the severe dehydration assay and the soil drought assay.

Potential applications. Based on the above results, G2156 may be used to improve tolerance to abiotic stress in commercial species.

G2156 (SEQ ID NO: 129 and 130; Arabidopsis thaliana)—Leaf RBCS3

Background. The aim of this project was to determine whether expression of G2156 from an RBCS3 promoter, which predominantly drives expression in photosynthetic tissue, would eliminate or alleviate the undesirable morphological effects of overexpression. We also wished to test whether G2156, when combined with an RBCS3 promoter, would result in increased abiotic stress tolerance.

Morphological Observations. Arabidopsis lines in which G2156 was expressed from the RBCS3 promoter (using the two component system) exhibited changes in size, altered leaf shape and in some cases showed delayed flowering. In particular, a substantial number of the lines developed enlarged leaves at later stages of growth. Details of lines and phenotypes are provided below:

T1 Lines 541-560: all were small at early stages and displayed rounded leaves with short petioles. Ten of the twenty lines (543, 544, 545, 546, 548, 552, 554, 557 558, 559) flowered later that controls to various extents and ten of the twenty lines (543, 544, 545, 548, 554, 550, 551, 554, 555, 557) developed distinctly enlarged leaves.

T1 Lines 581-587: all were small at early stages and displayed rounded leaves with short petioles. All showed varying degrees of late flowering, with #585 being most extreme. #584, 585, 586, 587 developed enlarged leaves.

Three T2 populations were later examined:

T2-543; all appeared wild type.

T2-544; all showed broad leaves.

T2-554; all had broad rather large curled leaves and enlarged flowers.

Physiology Results. Three out of ten lines of RBCS3::G2156 were less sensitive to ABA in a germination assay compared with wild-type control seedlings

Discussion. We have obtained RBCS3::G2156 lines using a two component approach. At early stages, these plants were slightly small and showed rather rounded leaves. However, at later stages, 50% of the lines developed enlarged leaves and showed increased rosette biomass compare to controls. The majority of lines showing this phenotype also displayed a slight delay in the onset of flowering.

Interestingly, large leaves were also observed when 35S::G2156 lines were re-examined. However, leaf enlargements were seen at lower frequency in the 35S::G2156 study than in the RBCS3::G2156 study. Additionally many of the lines from the 35S::G2156 experiment were very small and had multiple defects; such effects appear to have been largely avoided by use of the RBCS3 promoter. These differences could either be due to the possibility that differential expression solely in mesophyll cells does not produce toxic effects, or because of the different expression levels obtained using the two different promoters. The increased leaf size seen in this study was comparable to the effects produced by increased G1073 activity and serves to strengthen the conclusion that the two genes have related roles.

RBCS3 produces expression in relatively mature, photosynthesizing leaf tissue. Thus, G2156 when expressed at a relatively late stage of leaf development produced developmental signals that maintained leaf growth. However, there remains the possibility that G2156 triggered the production of developmental signals in mature leaves that were then transmitted to younger leaf primordia, and committed them to overgrowth at an early stage.

In plate based assays, a lower frequency of positive results were seen than with the 35S lines. However, RBCS3::G2156 lines did show an enhanced performance on ABA germination plates.

Potential applications. The RBCS3::G2156 combination produced a lower frequency of deleterious effects than the 35S::G2156 combination, and thus G2156 under the regulatory control of the RBCS3 promoter may be used to produce plants that are tolerant to drought and other abiotic stresses. However, the combination also showed less compelling results than 35S lines in drought-related stress assays.

G2157 (SEQ ID NO: 143 and 144; Arabidopsis thaliana)—Constitutive 35S

Background. G2157 (AT3G55560) is an Arabidopsis AT-hook protein that is a potential homolog of G1073. During the initial genomics screens, 35S::G2157 lines were found to show various pleiotropic developmental abnormalities such as dwarfing and changes in leaf shape. We have recently begun to analyze additional 35S::G2157 Arabidopsis lines as part of the G1073-related studies, since tomato lines overexpressing G2157 exhibited an increased biomass phenotype in a field screen.

Morphological Observations. A total of nineteen new 35S::G2157 lines (#301 and #321-338) have been obtained. The majority of these lines (all except #321, 326, 330, 338) showed a morphological phenotype: the plants were rather small and light in coloration at the rosette stage and exhibited broad rounded leaves with short petioles and rather wrinkled margins. Several lines have developed increased biomass and enlarged rosette leaves versus controls at later stages. Some of these plants have broad, curling, ruffled leaves that roll up at edges, slightly darker coloration, and are slightly late in developing.

Physiology (Plate assays) Results. Three of ten lines were more tolerant to severe desiccation in plate-based assays than were wild-type controls.

Discussion. We have now obtained new sets of 35S::G2157 lines. These plants show very similar morphologies to overexpression lines for some of the other genes in the G1073 study group, particularly G2156 and G2153. 35S::G2157 lines were typically small at early stages, and had short, broad leaves with curled margins. At later stages, an increase in leaf size and biomass became apparent relative to wild-type.

Potential applications. Based on the morphological changes seen in 35S::G2157 lines, this gene could be applied to manipulate plant development, enhance biomass, and improve drought tolerance.

G3399 (SEQ ID NO: 117 and 118; Oryza sativa)—Constitutive 35S

Background. G3399 is a closely-related ortholog of G1073. Phylogenetic analysis identifies G3399 along with G3400 as being the most closely related rice homologs to G1073 (in this study). The aim of this project is to determine whether overexpression of G3399 in Arabidopsis produces comparable effects to those of G1073 overexpression.

Morphological Observations. Overexpression of G3399 produced marked changes in Arabidopsis morphology including alterations in seedling vigor, size, leaf shape and flowering time. A marked increase in organ size was observed in a significant number of the lines.

An initial set of 35S::G3399 lines (321-340), harbored a construct P21269, which contained a one base pair sequence change (leading to an amino acid substitution, see seq. details) in the G3399 clone relative to the wild-type allele of the gene. Later sets of lines (381-400 and 401-420) were transformed with P21465, which carried an error-free G3399 clone. Both constructs produced similar morphological effects, as detailed below. Initially, it appeared as though P21269 lines showed a lower rate of dwarfing, but having obtained a second set of P21465 lines (401-420), this no longer appears to be the case.

Lines 321-340 (P21269): at the earliest stages, considerable size variation was apparent, with many the lines being slightly smaller than controls; this effect was particularly seen in #326, 327, 328, 329, 333, 336, 337, 339, 340. Two lines (#326 and 337) were very tiny and showed contorted curled leaves. Enlarged leaves and flowers were observed in eight of twenty lines (#323, 325, 331, 332, 334, 335, 336, 340), and this effect first became apparent at the end of the rosette stage. In addition to the increased organ size, a number of the lines also flowered late: #324, 331, 332, 334, 340.

Lines 381-400 (P21465): at the earliest stages, all lines appeared very small with abnormally shaped curled contorted leaves (#382, 384, 386, 394, 397, 398 were particularly strongly affected). At later stages of growth, enlarged leaves were apparent in 4/20 lines: #381, 389, 390 and 395.

Line 401-420 (P21465): at the seedling stages, 4/20 (#403, 407, 410, 413) lines displayed enhanced vigor and were larger than controls 401 is dead. Some lines (#405, 407-409, 411, 412, 417, 419) also had long, narrow cotyledons. Later, the majority of lines (402, 403, 404, 406, 407, 410, 411, 412, 413, 415-420) showed large, broad, slightly curled rosette leaves. A minority of lines (#408, 409, 414) were severely dwarfed and yellow in coloration with highly contorted leaves.

T2408; all plants were markedly small at early stages. AU were pale, late flowering, and showed round leaves with highly curled contorted margins.

Physiology (Plate assays Results. An initial set of 35S::G3399 lines (321-340), harbored a construct P21269, which contained a one base pair sequence change (leading to an amino acid substitution, see seq. details) in the G3399 clone relative to the wild-type allele of the gene. Several lines with this construct had more root hairs than controls.

Later sets of lines (381400 and 401-420) were transformed with P21465, which carried an error-free G3399 clone. Both constructs produced similar morphological effects, as detailed below. This construct also produced plants with more extensively developed roots (i.e., more root mass) and/or more root hairs. Three of ten of the P21465 lines also showed a marginally better performance than controls in a severe dehydration assay.

Initially, in morphology assays, it appeared as though P21269 lines showed a lower rate of dwarfing, but having obtained a second set of P21465 lines (401-420), this no longer appears to be the case.

Physiology (Soil Drought-Clay Pot) Summary. 35S::G3399 lines containing P21465 (an error-free G3399 clone) showed significantly better survival than controls in soil drought assays.

Three independent lines were tested; line 408 showed better survival on each of two plant dates, whereas line 412 showed better survival on one of two plantings.

TABLE 73
35S::G3399 drought assay results:

| | | | Mean | | | | |
|---|---|---|---|---|---|---|---|
| | | Mean | drought | p-value for | Mean | Mean | |
| | Project | drought | score | drought score | survival | survival | p-value for difference in |
| Line | Type | score line | control | difference | for line | for control | survival |
| 408 | DPF | 1.4 | 0.80 | 0.14 | 0.20 | 0.11 | 0.033* |
| 408 | DPF | 3.7 | 1.3 | 0.00052* | 0.75 | 0.19 | 0.0000000000000000021* |
| 412 | DPF | 1.0 | 1.2 | 0.54 | 0.21 | 0.15 | 0.32 |
| 412 | DPF | 2.1 | 0.50 | 0.0026* | 0.46 | 0.11 | 0.000000079* |

DPF = direct promoter fusion project
Drought scale: 6 (highest score) = no stress symptoms, 0 (lowest score; most severe effect) = extreme stress symptoms
*line performed better than control (significant at P < 0.11)

Discussion. 35S::G3399 lines have been obtained containing either of two different constructs. Both constructs produced similar morphological phenotypes; many of the lines were small at early stages, showed alterations in leaf shape, and had slightly delayed flowering. However a significant number of lines developed enlarged lateral organs (leaves and flowers), particularly at later stages.

35S::G3399 lines did not exhibit a striking overall improvement in stress tolerance in a number of the plate-based abiotic stress assays. However, three lines were more tolerant than controls in a severe dehydration assay, and a number of the lines showed more vigorous root development (increase root mass and root hair density) than wild-type when grown on control plates in the absence of a stress treatment. Importantly, two lines that exhibited improved performance in the severe dehydration assay each performed better than controls in at least one soil-based drought assay. Each of these lines (#408 and #412) produced dwarf plants, to varying degrees. Line #415, which had increased organ size (similar to results found with 35S::G1073 lines) was tolerant to dehydration in a plate assay but not to drought in a soil-based assay. The results with this rice gene demonstrate that enhanced drought tolerance in soil based assays for the G1073 group, is separable from the increased biomass phenotype that is obtained with G1073 itself.

Potential applications. Overexpression of G3399 in Arabidopsis produced both alterations in organ size, as well as enhanced stress tolerance, as was found with overexpression of G1073. These data show that proteins with a comparable activity to G1073 exist in monocots. Thus, G3399 may be used in monocots to regulate these traits. The current results do not indicate that G3399 offers any better utility for producing stress-tolerant plants without morphological alterations compared to G1073.

G3400 (SEQ ED NO: 123 and 124; Oryza sativa)—Constitutive 35S

Background. G3400 is a rice ortholog of G1073. Phylogenetic analysis identifies G3400 along with G3399 as being the most closely related orthologs to G1073 in this study. The aim of this project is to determine whether overexpression of G3400 in Arabidopsis produces comparable effects to those of G1073 overexpression.

Morphological Observations. 35S::G3400 lines exhibited highly pleiotropic effects on morphology, and showed changes in overall size, organ size, coloration, leaf shape, fertility and flowering time. At early stages most lines were severely dwarfed; some lines developed enlarged leaves and rather bushy inflorescences at late stages.

### Line Details

Line set 301-320: zero transformants obtained.

T1 lines 321-325: all showed a reduction in size at early stages, had short rounded curled leaves, and were slow developing compared to controls. These effects were strongest in line #321. Later, however, from the mid-inflorescence phase onwards, two of the lines (#322 and 323) developed leaves that were distinctly broader than in wild type. The flowers of these lines also were large compared to those of controls (seed from 3 lines: 321-323).

T1 line set 341-360: zero transformants obtained.

T1 lines 361-362; two tiny deformed transformants recovered. Both died at early stages.

T1 lines 381-400; at early stages all lines were tiny and many had deformed cotyledons and contorted, cup-shaped leaves. By a late stage of development #381, 383, 385, 386, 389, 392, 396, and 400 developed broad, large, curly leaves, and rather bushy inflorescences. The flowers on some plants also were somewhat large.

T2-321: all tiny with pale contorted leaves at all stages of development.

T2-332 & T2-323: all small, with pale, curled, upright, serrated leaves and delayed flowering. At later stages, leaves became rather enlarged, and inflorescences were bushy and had short internodes.

All three of the 35S::G3400 line tested out-performed controls in germination and growth assays involving cold temperatures. It was also noted that 35S::G3400 seedlings were pale with long narrow leaves.

Physiology (Soil Drought-Clay Pot) Summary. Two out of three independent 35S::G3400 lines showed a significantly better performance than controls in a single run of a split pot soil drought assay (plants from lines and controls together in same pot).

TABLE 74
35S::G3400 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 322 | DPF | 0.58 | 0.17 | 0.031* | 0.25 | 0.17 | 0.080* |
| 323 | DPF | 0.92 | 0.25 | 0.013* | 0.33 | 0.11 | 0.000073* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3400 overexpression lines were obtained at a low frequency, suggesting that the gene might have lethal effects when overexpressed at high levels. The lines that were obtained were small, slow developing, had curled leaves and showed complex developmental abnormalities at early stages. However, at later stages, some of the lines formed enlarged leaves and flowers.

It should be noted that the morphologically similar effects caused by overexpression of this rice gene versus G1073 and the Arabidopsis paralogs, indicate that they likely have related functions.

Three 35S::G3400 lines, all of which contained small plants with altered leaf morphology (contorted, serrated edges), were tested in plate-based abiotic stress assays and soil-based drought assays. All three lines showed improved performance in cold germination and growth assays, and two of the three lines had improved performance in the soil drought assays. This demonstrates that the stress tolerance phenotypes obtained with the G1073-group are independent of the increased organ growth phenotype that is most prevalent in G1073 lines.

Potential applications. Overexpression of G3400 in Arabidopsis produced both alterations in organ size, as well as enhanced stress tolerance, as was found with overexpression of G1073. These data show that proteins with a comparable activity to G1073 exist in monocots. Thus, G3400 may be used in monocots to regulate these traits. The current results do not indicate that G3400 offers any better utility for producing stress-tolerant plants without morphological alterations compared to G1073, and that if G3400 is used, it might need to be optimized by use of a non-constitutive promoter.

G3401 (SEQ ID NO: 135 and 136; Oryza sativa)—Constitutive 35S

Background. G3401 is a closely-related rice homolog of G1073. G3401 is more distantly related to G1073 than the rice sequences G3399 and G3400. G2153 and G1069 are the Arabidopsis sequences most closely related to G3401. The aim of this project was to determine whether overexpression of G3401 in Arabidopsis produces comparable effects to those of G1073 overexpression.

Morphological Observations. 35S::G3401 lines exhibited a pleiotropic morphology and showed alterations in leaf size, leaf shape, flowering time and overall plant size.

### Line Details

T1 lines 341-352:

The phenotypes seen in this batch of plants were rather variable, but alterations in size and leaf morphology were apparent. At the seedling stage, 6/12 plants (343, 345, 346, 349, 351, 352) were distinctly small and possessed rather narrow cotyledons. At this stage the other lines appeared wild type. Later, considerable size variation and changes in leaf shape were noted; in particular lines #343, 345, 346, 349 were tiny with rounded leaves. A number of lines also grew rather more slowly and bolted later than in wild type: #342, 347, 351, 352. A single plant (#344) was noted to have slightly larger leaves than controls, at the late rosette stage, but this effect was subtle.

Three T2 populations were examined:

T2-342, T2-347, T2-352; all were slightly small, showed delayed flowering and developed broad, rounded leaves.

Physiology (Plate assays) Results. 35S::G3401 seedlings were more tolerant to sucrose than controls in germination assays.

Physiology (Soil Drought-Clay Pot) Summary. Two out of three independent 35S::G3401 lines showed a significantly better performance than controls in a single run of a split pot soil drought assay (plants from lines and controls together in same pot).

TABLE 75
35S::G3401 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 347 | DPF | 1.8 | 0.75 | 0.056* | 0.35 | 0.13 | 0.022* |
| 352 | DPF | 2.1 | 0.50 | 0.0084* | 0.42 | 0.15 | 0.0055* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Twelve 35S::G3401 T1 lines were obtained-these plants showed a range of developmental changes including reduced size, slow growth, and altered leaf shape. A single line exhibited slightly enlarged leaves at late stages.

Three 35S::G3401 T2 lines were tested in soil-based drought assays. Lines #347 and #352 each performed better than controls, whereas line #342 performed similarly to controls. It is noteworthy that line #342 did not perform better than controls in any plate-based stress assay, whereas lines #347 and #352 performed better than controls in salt and high sucrose germination assays and soil-based drought assays, and line #352 also performed better in cold germination assays. AU three T2 lines were slightly small, showed delayed flowering and developed broad, rather rounded leaves. These results are very similar to those obtained with G2153, in terms of both morphological impact and stress tolerance.

Potential applications. These data indicate that G3401 could be used to modify stress tolerance in crop plants, and indicate that proteins with comparable activities to G2153 and G1073 are present in monocots. It will be useful to understand the relationship between G3401 protein expression level, morphological effect and stress effects in order to better optimize drought tolerance in crop plants while maintaining a wild-type morphology.

G3407 (SEQ ID NO: 133 and 134; Oryza sativa)—Constitutive 35S

Background. G3407 is a rice gene that is related to G1073. Phylogenetic analysis indicates that G3407 is most closely related to the Arabidopsis genes G1075 and G1076. The aim of this project was to determine whether overexpression of G3407 in Arabidopsis produces comparable effects to those of G1073 overexpression.

Morphological Observations. 35S::G3407 transformants exhibited an increase in size at the seedling stage compared to wild type controls, but at later stages appeared wild type. This effect was observed in approximately 50% of the primary transformants from a single set of T1 lines, as detailed below:

Lines 321-331: 5 out of 11 seedlings showed the above effects. The seedlings appeared to have cotyledons vertically oriented relative to wild-type, indicating that they might have altered light responses. At all other stages of development, however, no consistent differences in morphology to wild-type were observed.

Three lines were examined in the T2 generation and the majority seedlings from one of these (#302) were noted to be large at the 7 day stage. Occasional seedlings from the T2-301 population were also noted to be large.

It should be noted that transformants were obtained at only a relatively low frequency with this gene, and that, for unknown reasons, zero lines were isolated in each of two other selection attempts.

Discussion. 35S::G3407 seedlings showed a marked increase in size at early stages relative to wild-type. However, at later stages 35S::G3407 lines appeared wild type. It should be noted that we have observed increased seedling vigor with 35S::G1073 lines and in overexpression lines for other genes from the study group. Thus, G3407 appears to share some degree of activity with other G1073-like proteins.

35S::G3407 lines did not exhibit improved performance in any plate-based abiotic stress tolerance assays. However, relatively few transformants were obtained, suggesting that high level expression might be lethal, and that the recovered transformants may all have been expressing G3407 at a low level.

Potential applications. 35S::G3407 lines showed a marked increase in size at early stages, indicating that this gene may be used to enhance seedling vigor. This trait has enormous value for commercial crops; increasing the survivability of seedlings in the field can increase yield potential.

G3456 (SEQ ID NO: 131 and 132; Glycine max)—Constitutive 35S

Background. G3456 was included in the drought program as a soy AT-Hook gene closely related to G1073. The aim of this project was to determine whether overexpression of G3456 in Arabidopsis produces comparable effects to those of G1073 overexpression.

Morphological Observations. 35S::G3456 lines exhibited alterations in overall size, coloration, inflorescence architecture, leaf shape, and flowering time. In particular, at later stages of growth, a significant number of lines developed enlarged leaves and displayed increased biomass and delayed senescence relative to wild type controls.

Line Details:

T1 Lines 321-337: at early stages these plants appeared wild type. However, 3/17 lines (#329, 334, 335) were slightly small, had short internodes, and displayed curled leaves relative to controls. Later in development, four of seventeen lines (#323, 325, 328, 332) exhibited substantially larger rosettes than controls and also were dark in coloration. These plants also showed a slight delay in the onset of flowering.

T1 Lines 341-350: 2/10 lines (#348 and 350) displayed noticeably enlarged leaves. All lines were rather dark at late stages and had slightly short inflorescence internodes leading to a somewhat bushy architecture. Occasional plants, such as #349, exhibited floral defects.

T1 Lines 361-380: all plants were slightly larger and darker than controls at later stages. At early stages, these lines were wild type in appearance.

Three lines were also examined in the T2 generation:

T2-326: all were small at early stages with cupped cotyledons and leaf curling. All were slow growing, but by late stages had developed noticeably larger leaves than wild type. Senescence was also delayed in these plants.

T2-331: all were small at early stages with cupped cotyledons and leaf curling. All were slow growing and were rather short, bushy, and exhibited delayed senescence compared to wild type.

T2-337: all were late flowering, developed dark green, enlarged leaves, and senesced later than controls.

Physiology (Plate assays) Results. 35S::G3456 seedlings showed enhanced tolerance to sodium chloride in a germination assay and during chilling conditions in a growth assay (based on the lack of anthocyanin production). It should be noted that some of the 35S::G3456 seedlings were also observed to be small and vitrified on control plates, in the absence of stress treatments.

Physiology (Soil Drought-Clay Pot) Summary. Overexpression of G3456 produced a marked enhancement of drought tolerance in Arabidopsis. Three independent lines were tested in soil drought assays. Two of these lines (#331 and 337) showed significantly better survival than controls in each of two different plantings.

TABLE 76
35S::G3456 drought assay results:

| | | | Mean | | | | |
|---|---|---|---|---|---|---|---|
| | | Mean | drought | p-value for | Mean | Mean | |
| | Project | drought | score | drought score | survival | survival | |
| Line | Type | score line | control | difference | for line | for control | p-value for difference in survival |
| 331 | DPF | 4.4 | 1.2 | 0.00019* | 0.95 | 0.17 | 0.000000000000000000000023* |
| 331 | DPF | 3.5 | 1.4 | 0.0041* | 0.64 | 0.17 | 0.000000000000034* |
| 337 | DPF | 3.1 | 0.80 | 0.00025* | 0.54 | 0.071 | 0.00000000000031* |
| 337 | DPF | 3.0 | 1.0 | 0.014* | 0.56 | 0.12 | 0.0000000000014* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3456 lines exhibited a number of morphological alterations including changes in leaf morphology, size, and inflorescence architecture. In addition, a number of the lines showed delayed flowering, dark coloration, delayed senescence, and exhibited larger organs at later stages of development. It should be noted that these developmental effects were similar to those produced by Arabidopsis genes from the G1073 study group.

Overexpression of G3456 conferred improved abiotic stress responses, as some 35S::G3456 lines exhibited improved performance in high salt germination and in chilling growth assays. Three lines were tested in the soil drought assay, and two of the three lines had consistently, and very significantly, improved drought tolerance. Based on these results, G3456 has a similar activity to the Arabidopsis genes from the study group.

Potential applications. Overexpression of G3456 can provide effective drought tolerance, and is a good candidate for improvement of drought tolerance in crops. However, like G1073, substantial morphological effects are associated with G3456 overexpression, including delayed senescence (that is known to be problematic in 35S::G1073 soybean).

The developmental effects above indicate that the gene could be used also to modify traits such as flowering time, and organ size. The dark coloration exhibited by some of the lines could indicate increased chlorophyll levels; G3456 might therefore also impact photosynthetic capacity, yield, and nutritional value.

G3459 (SEQ ID NO: 121 and 122; Glycine max)—Constitutive 35S

Background. G3459 was included in the drought program as a soy AT-Hook gene related to G1073. The aim of this project was to determine whether overexpression of G3459 in Arabidopsis produces comparable effects to those of G1073 overexpression.

Morphological Observations. Overexpression of G3459 produced a spectrum of effects on Arabidopsis growth and development.

The majority of transformants exhibited morphological abnormalities including dwarfing, abnormally-shaped, curled leaves and cotyledons, slow growth, delayed flowering, short internodes, floral defects (such as failure of stamen development), and changes in coloration and branching pattern. At later stages, some of the lines were observed to develop rather broad rounded leaves.

A total of 36 lines (301-313, 321-335, 341-345, 361-373) were obtained, spanning four different plantings. The above phenotypes were observed, to varying extents in all of the transformants.

Physiology (Plate assays) Results. Five out of ten 35S::G3459 lines were more tolerant to sodium chloride in a germination assay. Another group of lines was more tolerant to heat in a germination assay. A further group of lines was more tolerant to cold temperatures during a growth assay; one line in this group had enhanced tolerance to sodium chloride, while two other lines in this group had enhanced heat tolerance on germination.

Discussion. 35S::G3459 lines produced adverse effects on plant growth and development, resulting in dwarfing, abnormally-shaped curled leaves and cotyledons, and other structural defects. At later stages, some of the lines exhibited delayed flowering and enlarged leaves, indicating that the G3459 protein shared some degree of activity with other proteins from the G1073 study group. However, severe floral defects were seen in 35S::G3459 lines. Few seed were obtained, suggesting that constitutive overexpression of G3459 is toxic in Arabidopsis. No soil drought assay was performed. However, improved tolerance to sodium chloride and heat germination, as well as seedling chilling tolerance, were observed in plate abiotic stress assays.

Potential applications. Overexpression of G3459 causes developmental changes and thus the gene may be used to regulate morphological traits. Thus, G3459 may be used to enhance abiotic stress tolerance traits.

Background. G3460 is a soy protein that is related to G1073, although it is most closely related to the Arabidopsis proteins G1075 and G1076. The aim of this project was to determine whether overexpression of G3460 in Arabidopsis produces comparable effects to those of G1073 overexpression.

Morphological Observations. Overexpression of G3460 in Arabidopsis produced striking morphological effects that included changes in leaf shape, altered flower morphology, and dramatic increases in vegetative biomass.

Phenotypes obtained in the three sets of 35S::G3460 lines isolated are detailed below.

Lines 301-312: 6/12 plants (#301, 306, 307, 308, 309, 312) were small (particularly at early stages), and had rather dark green leaves that were curled and in some cases serrated. These lines were markedly late flowering and exhibited a variety of non-specific floral abnormalities. Line 304 lacked these phenotypes, but exhibited enlarged leaves. The remaining lines showed some size variation, but otherwise, no consistent differences to wild type.

Lines 321-326: 3/6 plants (#321, 325, 326) had markedly broad leaves. #322, 324, 325 were late flowering and displayed dark green curled rosettes and floral abnormalities at late stages. #323 was small with curled contorted leaves.

Lines 341-358: considerable size variation was apparent at early stages. Later, 12/18 plants (#341, 344, 345, 346, 347, 352, 354, 355, 356, 357, 358, 359) were small (10-90% wild type size), late flowering and showed broad but severely twisted leaves and floral abnormalities. 5/18 lines (#342, 349, 350, 351, 353) lacked the severe leaf curling, but produced extremely enlarged leaves and were only mildly late flowering compared to wild type. A single line #348 was slightly early flowering.

T2-321: all small at early stages, curled leaves, dark green, and later flowering at later stages.

T2-324; all were markedly small with curled leaves and poor fertility.

T2-326; all small at early stages, 4/6 had enlarged leaves later on. 1/6 were tiny, 1/6 appeared wild type.

T2-307; all small, with curled leaves.

T2-308; all slightly small, with curled yellow leaves.

T2-309; all small, with curled yellowed leaves.

T2-343; 3/6 slightly early flowering, 3/6 appeared wild type.

T2-351; all small at early stages with curled leaves. Some evidence of early flowering was seen in this line. At later stages, leaves became wide and rounded.

T2-353; all small with extremely curled leaves at early stages. At later stages, leaves became broader and larger than in controls.

The above results indicate that G3460 produces complex effects on morphology that are likely to be influenced by subtle changes in transgene expression level. Additionally, parental phenotypes were not always maintained between generations. For example, both lines 351 and 353 had an initial primary transformant that was large and did not show severe leaf curling. However, severe leaf curling was apparent in the T2 progeny from those lines.

Physiology (Plate assays) Results. Four of ten 35S::G3460 seedlings were more tolerant to heat during a germination assay. Occasional lines also showed positive results in some of the other plate assays. Some of the 35S::G3460 seedlings were also small, pale and poorly developed when grown on control plates in the absence of a stress treatment.

Physiology (Soil Drought-Clay Pot) Summary. 35S::G3460 lines outperformed wild-type controls when tested in soil drought assays. Two of three independent lines showed significantly better survival than controls in two different plantings.

TABLE 77
35S::G3460 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 343 | DPF | 0.40 | 0.60 | 0.58 | 0.050 | 0.093 | 0.17 |
| 343 | DPF | 0.70 | 0.80 | 0.84 | 0.10 | 0.13 | 0.45 |
| 353 | DPF | 2.2 | 0.70 | 0.0060* | 0.31 | 0.12 | 0.00023* |
| 353 | DPF | 2.5 | 0.30 | 0.00031* | 0.43 | 0.029 | 0.0000000077* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3460 lines showed positive results when tested in plate based stress assays (heat tolerance on germination) and in the soil drought assay.

However, the majority of 35S::G3460 lines displayed a variety of morphological abnormalities including reduced size, slow growth, very delayed flowering, severely curled leaves and floral defects. Nonetheless, nine out of a total of thirty six T1 lines showed a somewhat different phenotype; these plants were slightly late flowering but developed extremely enlarged leaves, particularly at later stages of development. This resulted in a very substantial increase in vegetative biomass (possibly greater than that seen in 35S::G1073 Arabidopsis lines).

Interestingly, some T1 lines with enhanced vegetative biomass yielded small T2 progeny with extremely curled leaves. The basis of this change is unknown at present.

It is interesting to note that some aspects of the above phenotype, such as the enlarged leaves, were similar to those seen in 35S::G1073 lines. However, other features such as the extremely twisted dark green curled leaves seen in the majority of 35S::G3460 lines were not seen in 35S::G1073 transformants.

Potential applications. G3460 can provide enhanced drought tolerance in Arabidopsis, and thus could be used to regulate drought tolerance in crops. However, like G1073, G3460 produced undesirable morphological effects in Arabidopsis. It is not clear how these effects, which are somewhat different from those observed with G1073, might translate to soybean. However, particular with expression optimization, G3460 is an excellent candidate for the enhancement of yield and biomass accumulation.

G3408 (SEQ ID NO:145 and 146; Oryza sativa)—Constitutive 35S

Background. G3408 is more distantly related to G1073 than many of the other crop homologs in our study. The aim of this project was to determine whether overexpression of G3408 in Arabidopsis produces comparable effects to those of G1073 overexpression.

Morphological Observations. Overexpression of G3408 produced a number of alterations in Arabidopsis growth and development including changes in size, leaf shape, growth rate and flowering time. Interestingly, a number of T2 lines showed similar morphology to that seen in 35S::G1073 plants.

Physiology (Soil Drought-Clay Pot) Summary. 35S::G3408 lines were observed to be more tolerant to drought than control lines.

TABLE 78
35S::G3408 drought assay results.

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 331_MIX | DPF | 0.20 | 0.20 | 1.0 | 0.021 | 0.021 | 1.0 |
| 331_MIX | DPF | 1.9 | 1.3 | 0.059* | 0.34 | 0.26 | 0.12 |
| 331_MIX | DPF | 1.2 | 1.6 | 0.16 | 0.26 | 0.30 | 0.42 |
| 327 | DPF | 1.0 | 0.42 | 0.011* | 0.27 | 0.24 | 0.79 |
| 331 | DPF | 0.25 | 0.083 | 0.072* | 0.17 | 0.13 | 0.21 |
| 336 | DPF | 0.67 | 0.25 | 0.048* | 0.21 | 0.24 | 0.45 |

DPF = direct promoter fusion project
TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3408 lines exhibited a variety of morphological changes including reduced size, alterations in leaf shape, slow growth, and delayed flowering. Occasional lines showed broad, enlarged leaves. Such effects were comparable to those seen in some of the overexpression lines for the related Arabidopsis genes G2153 and G2156, suggesting that G3408 has a somewhat comparable activity to those proteins. While 35S::G3408 lines did not exhibit any consistently improved performance in either plate-based abiotic stress assays, they did perform better in our soil-based drought assays. It should be noted that the primary transformants were all dwarfed, although the T2 progeny used in physiology assays had relatively wild-type morphology. Thus, it is possible that G3408 was relatively poorly expressed in the lines subjected to abiotic stress assays.

Potential applications. Based on the results obtained so far, G3408 might be applied to modify developmental characters such as leaf morphology and flowering time, and improve drought tolerance.

Background. G1274 from Arabidopsis is a member of the WRKY family of transcription factors and is of interest based on soil drought tolerance exhibited by 35S::G1274 Arabidopsis lines. G1274 corresponds to AtWRKY51 (At5g64810).

Morphological Observations.

Direct promoter fusion lines: Twenty 35S::G1274 direct promoter-fusion lines have been isolated (Lines 301-320). Lines 301, 302, 304, 307, 308, 309, 311 and 313-320 had broad, rounded, somewhat flat leaves that were oriented upwards. The leaves of the other lines were wild type. Lines 302, 311 and 315 had a more extreme phenotype and exhibited a short stature with slightly bushy architecture. Late in development, most lines showed no differences from controls, although lines 302, 311 and 315 had bushier inflorescences and increased silique number. At a low frequency, especially noted in line 302, trilocular siliques with increased seed number were noted.

Two-component lines: Twenty-eight 35S::G1274 two component lines have been isolated (lines 321-328, 381-392 and 401-408. More extreme phenotypes were observed in these lines, compared to the direct promoter-fusion lines. Lines were typically late developing and were reduced in size to varying degrees. Leaves were dark green and plants typically had compact rosettes and bushy inflorescences. Five T1 plants died before maturation, and fertility was reduced in plants that did reach maturity.

Physiology (Plate assays) Results. Several 35S::G1274 lines (two-component and direct promoter fusions) from multiple generations were analyzed in plate-based abiotic stress assays. Direct promoter fusion lines (lines 1 and 4, T3 lines generated during our genomics program) showed sucrose insensitivity in a germination assay and tolerance to chilling growth conditions (lines 1, 4, 8, 12, 16). These lines also performed better than controls in a C/N sensing and a low N growth assay. However, no significant difference compared to wild-type seedlings was seen when a new set of T2 direct promoter fusion lines (302-314) were analyzed.

For the two-component lines that were examined, several were found to be insensitive to ABA in a germination assay and tolerant to chilling growth and severe dehydration. However, for unknown reasons, these lines did not show positive results in N related assays.

Several lines of both promoter types had less root growth, were chlorotic and small.

Physiology (Soil Drought-Clay Pot) Summary. 35S::G1274 lines showed excellent drought tolerance with both direct-fusion and two component lines across multiple lines and plant dates.

TABLE 79
35S::G1274 drought assay results:

| | | | | Mean | | | Mean | |
|---|---|---|---|---|---|---|---|---|
| | | | Mean | drought | p-value for | Mean | survival | |
| | | Project | drought | score | drought score | survival | for | p-value for difference in |
| PID | Line | Type | score line | control | difference | for line | control | survival |
| P8239 | 392 | TCST | 3.0 | 1.3 | 0.023* | 0.71 | 0.21 | 0.0000000000000024* |
| P8239 | 402 | TCST | 2.5 | 1.1 | 0.0047* | 0.36 | 0.24 | 0.027* |
| P8239 | 403 | TCST | 2.1 | 0.90 | 0.0022* | 0.50 | 0.17 | 0.000000020* |

Discussion. Plants containing direct fusion and two-component 35S::G1274 constructs showed excellent drought tolerance in our clay pot soil drought screens. Such results have been obtained in multiple lines across several plant dates. 35S::G1274 lines were also analyzed in plate-based abiotic stress assays. Direct promoter fusion lines showed sucrose insensitivity in a germination assay and tolerance to chilling growth conditions. Additionally, some of these lines showed a better performance than controls in a low N growth and C/N sensing assay on plates. However, in a new set of T2 direct promoter fusion lines, no significant difference compared to wild-type seedlings was observed. Several two-component lines were also examined. Several of these lines were found to be insensitive to ABA in a germination assay and tolerant to chilling growth and severe dehydration. The difference observed between direct fusion and two-component lines may have been due to expression level obtained in the various lines.

35S::G1274 plants consistently displayed a short, bushy architecture. This phenotype was stronger in two-component lines, where it was often accompanied by a delay in development. Leaves of 35S::G1274 plants were more rounded, flat and oriented upwards compared to wild-type. Additionally, at low frequency, some lines exhibited an overall increase in silique number, as well as a trilocular (and sometimes wrinkled) silique phenotype. These siliques contained an additional chamber of seeds, which sometimes resulted in twice as many seeds being produced, compared to wild-type controls. This silique effect was also observed in several of the G1274 crop homologs. However, some two-component lines showed an overall reduction in fertility.

Potential applications. Based upon soil drought, ABA, and sucrose plate assay results, G1274 appears to be an excellent candidate for improvement of drought/osmotic stress tolerance in crop species. G1274 also appears useful in protecting plants against low temperature, as well in low nitrogen environments. Additionally, G1274 may be optimized for use in increasing yield, or altering fruit architecture. However, some of the morphological effects associated with overexpression suggest that tissue-specific or conditional promoters might be required to enhance the utility of this and related genes.

G1274 (SEQ ID NO: 185 and 186; Arabidopsis thialiana)—Super Activation (C-GAL4-TA)

Background. The aim of this project was to determine whether the efficacy of the G1274 protein could be improved by addition of an artificial GAL4 activation domain at the C-terminus.

Morphological Observations. 35S::G1274-GA-4 lines showed somewhat comparable morphological effects to 35S::G1274 lines. The majority of plants showed an increase in leaf size. A number of lines also exhibited a loss of apical dominance during the reproductive phase. This latter phenotype was possibly less penetrant, though, than with 35S::G1274 lines.

Line Details: the following showed short, broad, flat leaves versus the control: #781, 784, 785, 787, 789-791, 795, 796, 799. #784, 787, 791, 799 exhibited a bushy phenotype.

Discussion. Several lines were examined morphologically, and showed somewhat comparable morphological effects to 35S::G1274. The majority of plants showed broad and flat leaves, leading to an overall increase in leaf size. A number of lines also exhibited a loss of apical dominance during the reproductive phase. This latter phenotype was possibly less penetrant than that seen with constitutive G1274 lines. A distinct effect on silique morphology was not noted in the lines thus far examined, as was seen in some 35S::G1274 plants.

It is notable that these 35S::G1274-GAL4 plants affect overall leaf morphology and inflorescence structure similarly to G1274. Thus, the GAL4 might also serve as a useful tag for use in immunoprecipitation experiments. At this time, it is unknown if fusion of the GAL4 activation domain will enhance stress tolerance.

G1274 (SEQ ID NO: 185 and 186; Arabidopsis thaliana)—Point Mutation

Background. G1274 from Arabidopsis is a member of the WRKY family of transcription factors and is of interest based on the soil drought tolerance exhibited by 35S::G1274 Arabidopsis lines. G1274 corresponds to AtWRKY51 (At5g64810).

The aim of this project was to examine the role of particular residues within the G1274 protein. The following clones have been engineered via site-directed mutagenesis:

(1) 35S::G1274 (K120Q)

(2) 35S::G1274 (N131S)

(3) 35S::G1274 (D149S)

(4) 35S::G1274 (Y155V)

(5) 35S::G1274 (S136T)

The K120Q mutation targets an amino acid within the highly conserved WRKY box of the G1274 DNA binding domain. The sequence of the WRKY box is highly conserved among all WRKY-family proteins, and it is notable that the three Arabidopsis proteins included in the G1274 study group (G1274, G1275 and G1758) have an amino acid variation within this box that distinguishes them from all other WRKY proteins. This K120Q mutation converts the WRKY box of these three proteins to a sequence similar to that found in all other WRKY proteins (which contain a Q residue at this position). Mutations 2-5 above each target strongly conserved amino acids within the DNA binding domain, but outside of the WRKY box. These residues, in effect, define the G1274 study group as distinct from the other WRKY II-c family members.

Morphological Observations. Overexpression lines for each of five different mutagenized variants of G1274 have now been generated. Interestingly these lines differentiated between the broad leaf and bushy inflorescence phenotypes that are characteristic of G1274 overexpression.

The first of the variants (K120Q) produced a much more extreme bushy phenotype than wild-type. Variants 2-4 ((N131S); (D149S); (Y155V)) each yielded increased leaf size with no effect or only a very mild effect on apical dominance. Variant 5 (S136T) produced equivalent effects to the wild-type form of the G1274 protein.

(1) Line 801-820(contains 35S::G1274(K120Q)): all were small at early stages, with upward pointing leaves, and a slightly pale coloration. During the reproductive phase, these lines showed a more extreme loss of apical dominance than overexpression lines for the wild-type G1274 clone. No changes in leaf size were apparent in these lines. Lines 801, 803, 804, 806, 808, 810-813, 815, 817 and 818 showed the strongest effects. #814 and #816 were slightly small and early developing versus wild-type. Lines 802, 807, 809, 817 were wild-type.

(2) Lines 821-840 (contains 35S::G1274(N131S)): all lines (except 839 and 840 which appeared wild type) showed broad leaves. A number of lines exhibited a slight loss of apical dominance during the reproductive phase.

(3) Lines 861-880 (contains 35S::G1274(D149S)): almost all showed broad flat leaves; two lines appeared wild type.

(4) Lines 881-900 (contains 35S::G1274(Y155V)): most showed broad flat leaves, a few lines were tiny. Several lines were slow developing.

(5) Lines 841-860 (contains 35S::G1274(S136T)): all showed broad flat leaves and a number showed a bushy phenotype. These effects were comparable to those seen on overexpression of the wild-type form of G1274.

Physiology (Plate assays) Results. Many of the lines harboring mutagenized variants of G1274 were more tolerant, relative to controls, in plate-based stress assays.

Lines 801-820 overexpressing site-directed mutation (1), were more tolerant to germination in cold conditions (10 of 10 lines tested) and growth in cold conditions (5 of 10 lines tested) than wild-type controls. Lines 801-820 also had altered C/N sensing, with 7 of 10 lines tested having increased tolerance to germination in low nitrogen conditions, and 10 of 10 lines having less anthocyanin on basal media minus nitrogen plus 3% sucrose and 1 mM glutamine.

Lines 821-840, overexpressing site-directed mutation (2), were more tolerant to germination in cold conditions (6 of 10 lines tested) and growth in cold conditions (5 of 10 lines tested) than wild-type controls. Lines 821-840 also had altered C/N sensing, with 5 of 10 lines having less anthocyanin on basal media minus nitrogen plus 3% sucrose and 1 mM glutamine.

Lines 861-880, overexpressing site-directed mutation (3), were more tolerant to germination in cold conditions (5 of 10 lines tested). Lines 861-880 also had altered C/N sensing, with 7 of 10 lines having less anthocyanin on basal media minus nitrogen plus 3% sucrose and 1 mM glutamine.

Lines 881-900, overexpressing site-directed mutation (4), were more tolerant to sucrose (5 of 10 lines tested), germination in cold conditions (3 of 10 lines tested), growth in cold conditions (3 of 10 lines tested), and less sensitive to ABA (6 of 10 lines tested), than wild-type controls. Under low N conditions, 3 of 10 lines exhibited better root growth than wild-type control plants.

Four of ten lines 841-850 overexpressing site-directed mutation (5) were more tolerant to desiccation than control plants in plate-based assays.

Physiology (Soil Drought-Clay Pot) Summary. Lines harboring mutagenized variants of G1274 generally showed strongly enhanced survival relative to wild-type in soil drought assays. For each of the first for variants, at least one line showed a better rate of survival and/or recovery from drought treatment than wild-type controls.

TABLE 80
G1274 point mutation drought assay results:

| | | Mean | Mean | | | | |
|---|---|---|---|---|---|---|---|
| | | drought | drought | p-value for | Mean | Mean | p-value for |
| | | score | score | drought score | survival for | survival for | difference in |
| Line | Project Type | line | control | difference | line | control | survival |
| 809 | Site Dir Mut 1 | 3.9 | 2.9 | 0.055* | 0.88 | 0.57 | 0.0000014* |
| 809 | Site Dir Mut 1 | 1.7 | 1.3 | 0.26 | 0.43 | 0.26 | 0.0023 |
| 824 | Site Dir Mut 2 | 1.9 | 2 | 1 | 0.37 | 0.41 | 0.49 |
| 824 | Site Dir Mut 2 | 1.6 | 1 | 0.085* | 0.36 | 0.21 | 0.0069* |
| 862 | Site Dir Mut 3 | 1.9 | 0.88 | 0.03* | 0.3 | 0.25 | 0.37 |
| 862 | Site Dir Mut 3 | 1.6 | 0.9 | 0.015* | 0.34 | 0.16 | 0.0011* |
| 865 | Site Dir Mut 3 | 1.8 | 0.75 | 0.033* | 0.32 | 0.21 | 0.05* |
| 865 | Site Dir Mut 3 | 1.5 | 1.6 | 0.87 | 0.29 | 0.31 | 0.69 |
| 866 | Site Dir Mut 3 | 0.88 | 0.25 | 0.017* | 0.25 | 0.098 | 0.0037* |
| 866 | Site Dir Mut 3 | 2.2 | 1.4 | 0.026* | 0.39 | 0.39 | 0.90 |
| 885 | Site Dir Mut 4 | 3.1 | 1.4 | 0.0018* | 0.82 | 0.38 | 0.00000000000065* |
| 885 | Site Dir Mut 4 | 1.7 | 1.2 | 0.14 | 0.40 | 0.35 | 0.39 |

Site Dir Mut (no.) = Site directed mutation project no.
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. The point mutants made for this project have separated the broad leaf and bushy inflorescence phenotypes that are characteristic of G1274 overexpression. The K120Q variant exacerbated the bushy phenotype, with the plants showing an extreme loss of apical dominance. The N131S, D149S and Y155V variants, on the other hand, each yielded increased leaf size with no effect or only a very mild effect on apical dominance. The final variant, S136T, produced equivalent effects to the wild-type form of the G1274 protein.

Arabidopsis lines ectopically expressing site directed mutation projects 1, 2, 3 and 4 each showed significant drought tolerance in one or more plantings relative to controls.

Potential applications. The variants tested here indicate a means of using G1274 to release apical dominance without affecting leaf size and vice versa. Various mutations shed light on the role played by specific residues in conferring abiotic stress tolerance in the G1274 polypeptide, and may be used to confer drought tolerance.

G1275 (SEQ ID NO: 207 and 208; Arabidopsis thaliana)—Constitutive 35S

Background. G1275 from Arabidopsis is a member of the WRKY family of transcription factors and is of interest based on its high similarity to G1274, which exhibited soil drought tolerance when overexpressed. G1275 corresponds to AtWRKY50 (At5g26170). In our earlier genomics program, G1275 overexpression was noted to cause marked dwarfing and loss of apical dominance. No indication of stress tolerance was observed in the assays run at that time (in contrast to G1274, which did show some chilling and low nitrogen tolerance). The aim of this project was to determine whether expression of G1275 from the 35S promoter was sufficient to confer stress tolerance that is similar to, or better than, that seen in 35S::G1274 lines.

Morphological Observations. G1275 induced severe dwarfing, and significant lethality when overexpressed. Approximately half of the T1 seedlings isolated died or were infertile. The degree of dwarfing in 35S::G1275 was variable; however, all T1 plants were reduced in size compared to controls. Inflorescences were typically compact and bushy, and lines were also late-flowering to various degrees. Line 365 exhibited floral abnormalities and line 364 had wrinkled siliques. A total of 31 lines were isolated from three batches of T1 plants: Lines 301-303, 361-374 and 521-536. Due to the morphological abnormalities, only very limited quantities of seeds were available for stress assays.

Physiology (Plate assays) Results. Four out of eight 35S::G1275 lines were larger and greener than control seedlings in a heat germination assay. Four lines were also more tolerant than wild-type seedlings to cold conditions in a chilling growth assay.

Discussion. Several direct fusion lines have been examined in plate-based assays. These lines were noted to be tolerant of both heat and cold stress. Morphologically. 35S::G1275 plants consistently displayed a short, bushy architecture. This phenotype was similar to that seen in 35S::G1274 plants, but stronger here and included some severe dwarfing and lethality. Also similar to what was seen when G1274 was overexpressed, 35S::G1275 plants also showed a wrinkled silique phenotype (although siliques containing extra locules and/or seeds have not been noted in the lines examined). Some plants had rounded and flat leaves, similar to that seen with G1274 overexpression.

Potential applications. 35S::G1275 plants have yet to be tested in drought assays. However, based on the plate assay data, G1275 could be used to create temperature stress-tolerant plants. G1275 appears to similarly affect overall morphology and plant architecture as did G1274, but some of the potentially negative effects are more pronounced with G1275. Therefore, this gene may need to be optimized using tissue-specific or conditional promoters to enhance utility. The gene might also be applied to regulate morphological traits such as flowering time, branching patterns, and fruit development.

G1275 (SEQ ID NO: 207 and 208; Arabidopsis thaliana)—Stress Inducible RD29A—line 5

Background. The aim of this project was to determine whether expression of G1275 from an RD29A stress inducible promoter, was sufficient to confer stress tolerance without the deleterious morphological off-types (loss of apical dominance, dwarfing etc.) that are associated with G1275 constitutive expression

Morphological Observations. A total of thirty-two RD29A_line5::G1275 lines were isolated from four separate batches of T1 plants (441-447, 461-463, 481-491 and 501-511). In general plants were slightly smaller than controls. Four plants died early in development. Lines 461, 463 and 483 were late flowering, and line 505 had siliques that were broader than control siliques. At 31 days, lines 501 and 507 had long rectangular rosette leaves.

Physiology (Plate assays) Results. Three out of ten RD29A::G1275 lines were insensitive to ABA in a germination assay.

Physiology (Soil Drought-Clay Pot) Summary. RD29A::G1275 lines showed strongly enhanced survival relative to wild-type in soil drought assays. Two lines showed a better rate of survival than wild-type controls and one line exhibited significantly better survival than wild-type on one plant date.

TABLE 81
RD29A::G1275 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 444 | TCST | 4.4 | 3.6 | 0.039* | 0.87 | 0.71 | 0.0017* |
| 444 | TCST | 1.5 | 1.5 | 0.86 | 0.26 | 0.35 | 0.12 |
| 463 | TCST | 2.4 | 2.8 | 0.28 | 0.47 | 0.52 | 0.40 |
| 463 | TCST | 2.0 | 1.2 | 0.0075* | 0.28 | 0.24 | 0.41 |

TCST = Two component super transformation project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. RD29A::G1275 lines have been obtained using a two-component system. In plate-based stress assays, 30% of lines tested were insensitive to ABA. Two lines showed evidence of greater tolerance to drought than controls. Morphologically, a few lines showed sporadic differences in leaf or silique shape, but overall, the plants did not show consistent differences from controls, other than a slight reduction in overall size.

Potential applications. Based on the results from plate assays, G1275 may be used along with an inducible promoter to confer tolerance to abiotic stresses such as drought.

G194 (SEQ ID NO: 217 and 218; Arabidopsis thaliana)—Constitutive 35S

Background. G194 (AtWRKY23, At2g47260) lies just outside of the phylogenetically defined G1274 study group. This gene was included in the present study to test the functional boundary of the G1274 study group, and determine the range of effects caused by genes that are more distantly-related to G1274. G194 is very closely related to G2517, which produced similar stress tolerance effects as G194 when overexpressed.

Morphological Observations. G194 produced severe dwarfing when overexpressed. The most severely affected individuals died at early stages. T1 plants were also typically late flowering, and approximately one third of the T1 plants died during the course of analysis. Lines overexpressing G194 were also typically delayed developmentally, and floral abnormalities were common on lines that produced flowers.

A total of twenty T1 lines were obtained: 301-311 and 321-329. Lines 308, 309, 322, 323, 325 and 328 were tiny (<5% of control). Line 325 was also slightly dark green and had a compact rosette. Line 321, 324, 326, 327, 329 and 330 were small (<50% of control size).

Physiology (Plate assays) Results. Four out of four 35S:G194 lines performed better than wild-type seedlings in a severe dehydration assay.

Discussion. 40% of the 35S::G194 lines tested in a plate-based severe dehydration assay were more tolerant than controls. The closely related gene, G2517, behaved similarly in this same assay. The most consistent morphological effect from G194 overexpression was severe dwarfing (5-50% of control size). Additionally, plants were late flowering, and many T1 plants died during the course of analysis. When plants survived to the reproductive stage, floral abnormalities were also common. These phenotypes are much more severe than those observed for G1274. The tolerance to severe dehydration indicates that more distant WRKY proteins such as G194, have some activity in common with G1274. Nonetheless, that these proteins did not yield the same morphologies as G1274 when overexpressed indicates that they are not of equivalent function.

Potential applications. Several G194 overexpression lines demonstrated enhanced survival in the plate-based dehydration assay, so it is possible that G194 may be useful in creating drought tolerant plants. However, the severe morphological effects associated with overexpression would require optimizing with tissue-specific or conditional promoters to enhance any possible utility.

G1758 (SEQ ID NO: 393 and 394; Arabidopsis thaliana)—Constitutive 35S

Background. G1758 corresponds to Arabidopsis AtWRKY59 (At2g21900). The aim of this project was to re-assess with a greater number of lines whether expression of G1758 from the 35S promoter was sufficient to confer stress tolerance that is similar to, or better than, that seen in 35S::G1274 lines.

Physiology (Plate assays) Results. In stress plate assays, 3 of 10 lines were more tolerant than wild-type seedlings in a chilling growth assay. Morphologically, 35S::G1758 lines were not consistently different from wild-type.

Discussion. The gene was included in this research program based on its high similarity to G1274, which exhibited soil drought tolerance when overexpressed. Interestingly, though, during our earlier genomics program, 35S::G1758 lines did not show the morphological effects seen in 35S::G1274 and 35S::G1275 lines, and showed a wild-type response in the limited number of stress assays performed at that time.

Potential applications. Based on our analysis of 35S::G1758 lines, this gene may be used in creating plants that are tolerant of abiotic stresses such as cold.

G2517 (SEQ ID NO: 219 and 220; Arabidopsis thaliana)—Constitutive 35S

Background. G2517 (AtWRKY68, At3g62340) lies just outside of the phylogenetically defined G1274 study group. This gene was included in the present study to test the functional boundary of the G1274 study group, and determine the range of effects caused by genes that are more distantly-related to G1274. G2517 is very closely related to G194, which produced similar stress tolerance effects as G2517 when overexpressed.

Morphological Observations. G2517 induced early flowering when overexpressed. Plants were typically small with spindly inflorescences. Most plants were also early flowering, although a late flowering phenotype was observed in two cases (lines 363 and 365). A total of 54 35S::G2517 lines were isolated from 4 separate plantings.

Physiology (Plate assays) Results. Six out of ten 35S::G2517 lines were more tolerant to a severe dehydration based plate assay.

Discussion. 60% of the 35S::G2517 lines tested in a plate-based severe dehydration assay were more tolerant than controls. The closely related G194 behaved similarly in this same assay. G2517 overexpression also induced early flowering and spindly architecture. Plants were typically small, and at a low frequency, were late flowering. These morphological effects are similar to those seen in G194, and in general, are more severe than observed for G1274, G1274. The tolerance to severe dehydration indicates that more distant WRKY proteins such as G2517, have some activity in common with G1274. Nonetheless, that these proteins did not yield the same morphologies as G1274 when overexpressed indicates that they are not of equivalent function.

Potential applications. Several G2517 overexpression lines demonstrated enhanced survival in the plate-based dehydration assay, so G2517 may be used to create drought-tolerant plants. The morphological effects associated with overexpression may require optimizing with tissue-specific or conditional promoters. The gene might also be used to modify developmental traits such as flowering time.

G3719 (SEQ ID NO: 211 and 212; Zea mays)—Constitutive 35S

Background. G3719 is a WRKY gene from Zea mays and is a closely-related homolog of G1274. Based on our phylogenetic analysis, there are two separate clades representing potential monocot homologs of G1274, and this gene is most closely related to G3730 from rice. The aim of this study was to assess the role of G3719 in drought stress-related tolerance, and to compare the overexpression effects with those of other G1274-related genes.

Morphological Observations. G3719 induced severe dwarfing, a short bushy phenotype, significant lethality and various developmental alterations when overexpressed in Arabidopsis. 35S::G3719 seedlings were observed to be smaller than control seedlings. The majority of the plants died prior to maturation, and the plants surviving to adulthood typically had low fertility. A total of thirty-three 35S::G3719 lines have been selected from four separate planting dates (lines 301-306, 321-333, 361-368 and 381-386), but the seed yield from these plants was extremely poor.

Discussion. Morphological observations indicate that overexpression of this gene causes severe dwarfing, significant lethality and various developmental alterations such as a loss of apical dominance and low fertility. These effects are stronger than those seen in 35S::G1274 lines, and very similar to those observed for G1275 lines. This may indicate that the monocot subclade to which G3719 belongs may better represent G1275, rather than G1274.

35S::G3719 lines have not yet been tested in drought-related assays.

Potential applications. Alterations of apical dominance or plant architecture could create new plant varieties. Dwarf plants may be of potential interest to the ornamental horticulture industry, and shorter, more bushy plants may also have increased resistance to lodging.

G3720 (SEQ ID NO: 203 and 204; Zea mays)—Constitutive 35S

Background. G3720 is a WRKY gene from Zea mays and is a closely-related homolog of G1274. Based on our phylogenetic analysis, there are two separate clades representing potential monocot homologs of G1274, and this gene is most closely related to G3725 and G3726 from rice, and G3722 from corn. The aim of this study was to assess the role of G3720 in drought stress-related tolerance, and to compare the overexpression effects with those of other G1274-related genes.

Morphological Observations. G3720 induced severe dwarfing, loss of apical dominance and significant lethality and various developmental alterations when overexpressed in Arabidopsis. 35S::G3720 seedlings were observed to be smaller than control seedlings. The majority of the plants died prior to maturation, and the plants surviving to adulthood typically had low fertility. A total of thirty-seven 35S::G3720 lines have been selected from four separate planting dates (Lines 301-304, 321-333, 341-350 and 361-380), but these lines yielded few if any seeds.

Physiology Results. Three of ten lines tested showed more root growth under low nitrogen conditions than wild-type control plants.

Discussion. Morphologically, 35S::G3720 lines showed similar, but more severe phenotypes than the other G1274-related genes. These included significant mortality, dwarfing, extreme loss of apical dominance, as well as changes in flowering time and leaf shape. Additionally, these lines had decreased fertility. These severe phenotypes are also observed in overexpression lines of G3726 from rice, which is the most similar gene to G3720 in the study group. The 35S::G3720 effects were more similar to those of G1275 overexpression than G1274 overexpression.

Potential applications. The plate based results indicate that 35S::G3720 overexpression may be used to confer stress tolerance in plants, particularly to low nitrogen conditions. Alterations of apical dominance or plant architecture could create new plant varieties. Dwarf plants may be of potential interest to the ornamental horticulture industry, and shorter, more bushy plants may also have increased resistance to lodging.

G3721 (SEQ ID NO: 197 and 198; Oryza sativa)—Constitutive 35S

Background. G3721 is a WRKY gene from Oryza sativa and is a closely-related homolog of G1274. Based on our phylogenetic analysis, there are two separate clades representing potential monocot homologs of G1274, and this gene is most closely related to G3727, G3804, and G3728 from corn. The aim of this study was to assess the role of G3721 in drought stress-related tolerance, and to compare the overexpression effects with those of other G1274-related genes.

Morphological Observations. A total of twenty 35S::G3721 lines have been isolated: 301-320. Plants overexpressing G3721 were all initially small on selection plate, compared to controls. Seven days after germination they were also pale, vitrified and had very short roots. Six of twenty lines died shortly after transplantation and an additional seven lines were severely dwarfed. The severely dwarfed lines were typically spindly, bushy, and late-developing. They also had abnormal siliques. Line 313 was bushy and had terminal flowers emerging from the leaf axils. Lines 303 and 320 were wild-type sized and flowered earlier than controls.

Physiology (Plate assays) Summary. Seven of ten 35S::G3721 lines tested were more tolerant to salt, 5 of 10 lines were more tolerant to mannitol, 10 of 10 lines were less sensitive to ABA, and 10 of 10 lines were more tolerant to cold in a germination assay, than wild-type controls.

Discussion. Morphological observations indicate that overexpression of this gene causes dwarfing, loss of apical dominance, changes in flowering time and lethality. These effects are stronger than those seen in 35S::G1274 lines, and similar to those observed in overexpression lines for G1275, as well as the related rice gene G3730. At a low frequency, 35S::G3721 lines also showed an abnormal silique phenotype, similar to G1274 and other homolog lines.

Potential applications. The plate based results indicate that 35S::G3721 overexpression may be used to confer stress tolerance in plants, particularly to salt, cold, and hyperosmotic stresses, likely including drought. Alterations of apical dominance or plant architecture could create new plant varieties. Dwarf plants may be of potential interest to the ornamental horticulture industry, and shorter, more bushy plants may also have increased resistance to lodging.

G3722 (SEQ ID NO: 199 and 200; Zea mays)—Constitutive 35S

Background. G3722 is a WRKY gene from Zea mays and is a closely-related homolog of G1274. Based on our phylogenetic analysis, there are two separate clades representing potential monocot homologs of G1274, and this gene is most closely related to G3725 and G3726 from rice, and G3720 from corn. The aim of this study was to assess the role of G3722 in drought stress-related tolerance, and to compare the overexpression effects with those of other G1274-related genes.

Morphological Observations. A total of twenty 35S::G3722 T1 lines were analyzed (301-320). Three lines died early in development, and all additional lines were small, pale and curly-leafed in the vegetative phase. The size of the remaining 17 lines varied, but all were typically small and bushy compared to controls. Lines 302-304, 306 and 310-314 were early developing and spindly compared to wild-type (with lines 311 and 312 earliest). Lines 310 and 313 had abnormal siliques.

Physiology Results. Four of 10 35S::G3722 lines had a higher rate of germination in a low nitrogen germination assay (under low nitrogen and high sucrose conditions) than control plants, and 6 of 10 35S::G3722 lines demonstrated altered C/N sensing in a low nitrogen germination assay with glutamine as a nitrogen source.

Discussion. Morphologically, these lines showed similar phenotypes to the other G1274-related genes including lethality, dwarfing, loss of apical dominance, as well as changes in flowering time and leaf shape. At a low frequency, these lines also produced abnormal siliques.

Potential applications. The plate based results indicate that 35S::G3722 overexpression may be used to confer stress tolerance in plants, particularly to low nitrogen conditions. Alterations of apical dominance or plant architecture could create new plant varieties. Dwarf plants may be of potential interest to the ornamental horticulture industry, and shorter, more bushy plants may also have increased resistance to lodging.

G3724 (SEQ ID NO: 187 and 188; Glycine max)—Constitutive 35S

Background. G3724 is a WRKY gene from Glycine max and is a closely-related homolog of G1274. Based on our phylogenetic analysis, this gene is most closely related to G3723 and G3803, also from soy. The aim of this study was to assess the role of this gene in drought stress-related tolerance, and to compare the effects with those of other G1274-related genes.

Morphological Observations. A total of seventeen 35S::G3724 lines were isolated and analyzed (Lines 301-317). At early stages, the majority of lines were small and a number of lines were slightly early flowering (#312, 314, 315). A number of lines developed enlarged rosettes by the later stages of development: #305, 308, 310, 311, 317. Lines 310, 311 and 317 were late flowering.

Physiology results. Five of 10 lines tested were more tolerant to salt, 3 of ten lines were more tolerant to sucrose, 10 of 10 lines were less sensitive to ABA, and 8 of 10 lines were less sensitive to cold in a germination assay than wild-type control plants.

Discussion. 35S::G3724 lines were consistently more tolerant to a number of abiotic stresses than controls. Morphological observations indicate that overexpression of this gene causes changes in flowering time, leaf shape and overall size. Many plants were small at early stages, but later some lines developed enlarged rosettes. Overall, there was not a strongly consistent phenotype. However, the large leaf phenotype was somewhat similar to that seen in 35S::G1274 lines.

Potential applications. However, based on the morphology results obtained so far, the gene may be used to modify developmental traits such as flowering time and leaf shape. This gene may also be used to increase biomass in plants, resulting in increased yield. Furthermore, the plate based results indicate that 35S::G3724 overexpression may be used to confer stress tolerance in plants, particularly to salt, cold during germination, and hyperosmotic stresses, including drought.

G3725 (SEQ ID NO: 213 and 214; Oryza sativa)—Constitutive 35S

Background. G3725 is a WRKY gene from Oryza sativa and is a closely-related homolog of G1274. Based on our phylogenetic analysis, there are two separate clades representing potential monocot homologs of G1274, and this gene is most closely related to G3720 and G3722 from corn, and G3726 from rice. The aim of this study was to assess the role of G3725 in drought stress-related tolerance, and to compare the overexpression effects with those of other G1274-related genes.

Morphological Observations. Fifteen 35S::G3725 lines have been isolated and analyzed (301-315). G3725 induced early flowering, reduced plant size slightly.

Physiology results. 35S::G3725 lines have more root growth compared with wild-type seedlings on control plates.

Discussion. In plate-based stress assays, G3725 overexpressing seedlings were noted to have more vigorous root growth on control plates, but behaved similarly to wild-type in all other assays. Morphologically, these lines showed mild phenotypes compared to the other G1274-related genes, and were similar to wild-type. Some lines, though, flowered slightly early and had slightly reduced size, but otherwise were similar to controls.

Potential applications. At this time, it is unknown how G3725 overexpression may affect drought tolerance. However, given the effects seen in roots in our plate based assays, the gene may be used to manipulate root development.

G3726 (SEQ ID NO: 201 and 202; Oryza sativa)—Constitutive 35S

Background. G3726 is a WRKY gene from Oryza sativa and is a closely-related homolog of G1274. Based on our phylogenetic analysis, there are two separate clades representing potential monocot homologs of G1274, and this gene is most closely related to G3720 and G3722 from corn, and G3725 from rice. The aim of this study was to assess the role of G3726 in drought stress-related tolerance, and to compare the overexpression effects with those of other G1274-related genes.

Morphological Observations. Thirty-seven 35S::G3726 lines were isolated. G3726 overexpression generally induced early flowering, short internode growth. Fifteen of thirty-seven lines isolated died prior to maturity. Plant size was typically reduced and plants were bushy compared to controls, although the degree of growth suppression and bushiness varied between lines. Siliques were also commonly smaller in the most severely affected 35S::G3726 lines.

Physiology (Plate assays) Results. Three 35S::G3726 lines contained less anthocyanins and were larger than control seedlings in a germination assay under cold conditions. One of these and two other lines performed better than controls in a cold growth assay.

Physiology (Soil Drought-Clay Pot) Summary. Two 35S::G3726 lines showed drought tolerance in our soil drought screens, including when CBF4 overexpressors (OEX), known to be more tolerant to drought than wild-type plants, were used as controls, as noted in the table below. Such results have been obtained with both direct-fusion and two component lines across multiple lines and plant dates.

TABLE 82
35S::G3726 drought assay results:

| | | | | | Mean | p-value for | | | |
|---|---|---|---|---|---|---|---|---|---|
| | | | | Mean | drought | drought | Mean | Mean | p-value for |
| | | | Project | drought | score | score | survival | survival for | difference in |
| PID | Line | Control | Type | score line | control | difference | for line | control | survival |
| P25211 | 309 | CBF4 | DPF | 1.3 | 0.8 | 0.22 | 0.14 | 0.14 | 0.86 |
| | | OEX | | | | | | | |
| P25211 | 309 | CBF4 | DPF | 1.8 | 0.9 | 0.014* | 0.27 | 0.13 | 0.0027* |
| | | OEX | | | | | | | |
| P25211 | 313 | CBF4 | DPF | 2.5 | 0.6 | 0.00024* | 0.27 | 0.086 | 0.00014* |
| | | OEX | | | | | | | |
| P25211 | 313 | CBF4 | DPF | 2.1 | 0.9 | 0.00057* | 0.24 | 0.17 | 0.22 |
| | | OEX | | | | | | | |

DPF = Direct promoter fusion
OEX = overexpressor
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Of the ten lines tested in plate assays, 30% were larger and had less anthocyanin than control seedlings in a cold germination assay. One of these lines, along with two other lines contained less anthocyanins in a cold growth assay. Significantly, two lines were more tolerant to drought than controls overexpressing CBF4, which are plants known to be more tolerant to drought than wild-type.

Morphologically, these lines showed similar, but more severe phenotypes than the other G1274-related genes. These included significant mortality, dwarfing, extreme loss of apical dominance, as well as changes in flowering time and leaf shape. Additionally, these lines had decreased fertility. These severe phenotypes are also observed in overexpression lines of G3720 from corn, which is the most similar gene to G3726 in the study group.

Potential applications. The morphological phenotypes observed in these lines may indicate that expression of G3726 may require optimization with tissue-specific or conditional promoters to enhance potential utility in drought tolerance or plant architecture. The gene may be used to regulate developmental traits such as flowering time and branching. Results from cold stress plate and soil drought assays indicate that this gene may be a good candidate for increasing tolerance to low temperature and drought conditions.

G3804 (SEQ ID NO: 191 and 192; Zea mays)—Constitutive 35S

Background. G3804 is a WRKY gene from Zea mays and is a closely-related homolog of G1274. Based on our phylogenetic analysis, there are two separate clades representing potential monocot homologs of G1274, and this gene is most closely related to G3727 and G3728 from corn. The clone obtained for G3804 may actually represent a variant of G3728. The aim of this study was to assess the role of G3804 in drought stress-related tolerance, and to compare the overexpression effects with those of other G1274-related genes.

Morphological Observations. A total of twenty 35S::G3804 lines were isolated (301-320). G3804 induced dwarfing, poor root growth on selection plates, altered flowering-time and lethality when overexpressed in Arabidopsis. Plants were typically small and bushy compared to controls; however the phenotype varied among lines. Many of the lines showed flower buds sooner than wild-type, but then developed more slowly than wild-type during the inflorescence phase. Dues to the dwarfing in these lines, seed yield was very poor. However, lines 302 and 319 had trilocular siliques.

Physiology (Plate assays) Results. Four of ten 35S::G3804 lines were more tolerant to cold in a germination assay compared to wild-type seedlings.

Physiology (Soil Drought-Clay Pot) Summary. 35S::G3804 lines showed an excellent performance in soil drought screens. Each of three independent lines performed better than wild-type on each of two different plant dates.

TABLE 83
35S::G3804 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 304 | DPF | 3.5 | 0.70 | 0.00016* | 0.60 | 0.21 | 0.00000000015* |
| 304 | DPF | 3.5 | 1.5 | 0.0043* | 0.71 | 0.46 | 0.000028* |
| 307 | DPF | 2.6 | 1.3 | 0.0015* | 0.56 | 0.34 | 0.00022* |
| 307 | DPF | 2.5 | 1.7 | 0.088* | 0.74 | 0.55 | 0.00083* |
| 309 | DPF | 3.0 | 1.1 | 0.0020* | 0.60 | 0.29 | 0.00000028* |
| 309 | DPF | 2.7 | 0.90 | 0.0017* | 0.51 | 0.25 | 0.000013* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3804 lines showed an excellent performance in soil drought assays. Additionally 40% of the lines tested in a cold germination assay were more tolerant than wild-type controls. Morphologically, these lines showed similar phenotypes as the other G1274-related genes including lethality, dwarfing, loss of apical dominance, as well as changes in flowering time and leaf shape. At a low frequency, these lines also produced trilocular siliques containing more seeds than wild-type. Based on these phenotypes, G3804 has a comparable activity to the G1274 protein.

Potential applications. Based on the results obtained, G3804 could be applied to produce tolerance to abiotic stresses such as cold and drought. However, the morphological phenotypes observed in these lines may indicate that expression of G3804 could require optimization with tissue-specific or conditional promoters to enhanced potential utility in drought tolerance or to increase yield. The gene might also be used to regulate developmental traits such as flowering time, branching, fruit development and seed yield.

### The G1792 Clade

G1792 (SEQ ID NO: 221 and 222; Arabidopsis thialiana)—Constitutive 35S

Background. G1792 overexpressing lines showed enhanced tolerance to drought in a soil drought assay and enhanced resistance to multiple pathogens. 35S::G1792 lines were also more tolerant to low nitrogen conditions. We have assigned the name TDR1 (Transcriptional regulator of the Defense Response 1) to this gene, based on its apparent role in disease responses.

Morphological Observations. Twenty 35S::G1792 direct fusion lines (Lines 301-320) and thirty-seven 35S::G1792 two-component lines (Lines 401-420 and 521-537) were isolated. The overexpression of G1792 consistently induced dwarfing and dark green coloration in Arabidopsis. The effects of overexpression were more severe in the two-component lines, as dwarfing occurred earlier (at 7 days), and the degree of dwarfing in later stages of development was also more generally more pronounced.

Although all plants were smaller than wild-type there were size variations evident in the both the direct fusion and two component T1 plants. Flowering time was consistently late in the transgenic lines, and the leaves of the overexpression lines were shiny compared to the controls. One direct fusion line (302) was grayish green, and had flat leaves.

Physiology (Plate assays) Results. 35S::G1792 lines (direct promoter fusion and two component) had a better performance in a C/N sensing assay and growth under low N compared with wild-type seedlings. In addition, some direct promoter and two component lines showed greater tolerance to severe dehydration and cold conditions than wild type controls in growth assays.

Discussion. Both two-component and direct promoter fusion 35S::G1792 lines have been established. Overexpression of G1792 consistently induced dwarfing and dark green coloration in Arabidopsis. The degree of dwarfing was generally more severe in the two-component lines. Flowering time was consistently late in the transgenic lines, and the leaves of the overexpression lines were shiny compared to the controls.

Consistent with results obtained in the genomics program, 35S::G1792 lines had better performance in a C/N sensing assay and in root growth under low nitrogen than wild-type controls. In particular, under low N conditions, 35S::G1792 lines exhibited much better root growth and a higher density of root hairs compared to wild type. In addition, some lines showed tolerance to severe dehydration and cold conditions in plate based assays, phenotypes that were not uncovered in the genomics program. Seven independent lines have performed better than wild-type in the clay pot soil drought screen in one or more plant dates; one line (#12) performed significantly worse on two occasions.

Five of eight 35S::G1792 lines tested were significantly more resistant to Botrytis in a plate assay. No lines tested showed enhanced resistance to Sclerotinia, consistent with previous observations in the genomics program.

Potential applications. The results obtained to date indicate that G1792 and its related genes have a number of potential applications in crop plants, including improving tolerance to drought and other abiotic stresses, enhancing growth on limiting nitrogen, and increasing disease resistance, if expressed under appropriate promoters. Additionally, G1792 could be used to manipulate developmental traits such as flowering time, wax deposition, leaf shape, and root development.

G1792 (SEQ ID NO: 221 and 222; Arabidopsis thaliana)—Leaf-Specific RBCS3

Background. The aim of this project was to determine whether expression of G1792 from an RBCS3 promoter, which drives expression in photosynthetic tissues, is sufficient to confer drought tolerance or pathogen resistance, and whether restricting G1792 expression to these tissues can overcome the negative side effects (stunting, late development) of G1792 expression.

Morphological Observations. Twenty RBCS3::G1792 lines were isolated (361-380). Lines 361-369, 371, 375 and 376 were slightly small in size and slightly dark in coloration. All other lines were equivalent to control lines. Lines 368, 369, 371, 372 and 374-377 were late developing.

Physiology Results. Three out of ten RBCS3::G1792 lines performed better than wild-type seedlings when germinated in the presence of sodium chloride.

Disease Summary: Five RBCS3::G1792 two-component lines were tested in Sclerotinia and Botrytis plate assays. Three of these lines (373, 378, and 379) were more resistant than comparable control plants to Botrytis. No change in Sclerotinia resistance was observed in any of the lines.

N.B. TDR1 (Transcriptional regulator of the Defense Response) is the gene name for G1792. The control used in this experiment was the RBCS3 promoter background supertransformed with a GUS target gene.

Discussion. The majority of RBCS3::G1792 lines were slightly smaller, darker green, and later developing than controls, but these phenotypes were much less severe than those of 35S::G1792 plants. Three out of ten lines showed enhanced tolerance to sodium chloride in a germination assay. These lines were tested in the soil drought assay, but did not show enhanced drought tolerance. Three of five lines tested in disease assays showed enhanced resistance to Botrytis.

Potential applications. The results obtained to date indicate that expression of G1792 in photosynthetic tissue may enhance disease resistance and tolerance to abiotic stresses such as high salt, while alleviating the deleterious effects on morphology that are associated with constitutive expression.

G1792 (SEQ ID NO: 221 and 222; Arabidopsis thaliana)—GR Fusion (Dex Inducible)

Background. Two-component G1792 dexamethasone-inducible lines were created. Since G1792 produces dwarfing when overexpressed, the dexamethasone-inducible lines were generated to allow us to test lines in disease assays after dexamethasone application. N-terminal and C-terminal direct GR fusions to G1792 have also been created. If it is demonstrated that the fusion proteins are functional, these lines could also be used in microarray experiments.

Morphological Observations. The following sets of dexamethasone inducible lines have been generated:

Two-component lines:

Lines 321-340:

All T1 lines appeared wild type in the absence of dex, except for 328 which was late flowering

A number of lines were morphologically examined in subsequent generations and all showed a wild-type phenotype in the absence of dexamethasone.

Homozygous T3 generation populations have now been established for three lines: 334, 337, 340.

Direct Fusion Lines:

Lines 681-700 (containing a 35S::G1792-GR fusion):

In the absence of dex, all T1 plants were small at early stages. A number of lines also were early flowering (#682, 684, 685, 688, 689, 691, 692, 694, 699, 700)

Lines 720-740 (containing a 35S::GR-G1792 fusion):

In the absence of dexamethasone, these T1 lines appeared wild type except for a number of lines that showed early flowering (#729, 732, 733, 735, 736 and 738-740)

Disease Summary Eight two-component dexamethasone (dex)-inducible G1792 lines were tested in Sclerotinia, Botrytis, and Fusarium plate assays. Seven of these lines showed moderate to strong resistance to Botrytis when grown on plates containing dex. Two lines were more resistant to Fusarium than wild-type controls. No change in Sclerotinia resistance was observed. These lines did not show enhanced Botrytis resistance when grown on regular (minus dex) plates. The control used in this experiment was the dex-inducible promoter background supertransformed with a GUS target gene.

Physiology (Plate assays) Results. Of the abiotic stress analyses performed thus far, 5 of 10 G1792-GR fusion (dex inducible) lines had greater tolerance to severe dehydration than wild type-controls. Four of 10 lines had more root growth under low nitrogen conditions than wild-type controls.

Discussion. The two-component dexamethasone-inducible lines that have been created are wild-type in morphology. Two-component dexamethasone (dex)-inducible G1792 lines showed moderate to strong resistance to Botrytis and Fusarium when grown on plates containing dex. No change in Sclerotinia resistance was observed (35S::G1792 overexpression also does not confer Sclerotinia resistance).

A number of lines carrying the G1792 N-terminal and C-terminal direct GR fusion constructs were early flowering. In addition, the C-terminal lines were also smaller at early growth stages

Potential applications. The results obtained to date indicate that expression of G1792 under an inducible promoter could be used to produce tolerance to desiccating conditions, and disease resistance, while mitigating the negative side effects of G1792 overexpression.

G1792 (SEQ ID NO: 221 and 222; Arabidopsis thaliana)—Point Mutation

Background. The aim of this project was to investigate the role of four key conserved amino acids in the “EDLL” domain, which is conserved in the C-terminal end of the G1792 protein and its paralogs and orthologs. Each mutation changes one of these conserved amino acids: site-directed mutation_{—}1, E119V; site-directed mutation_{—}2, D124G; site-directed mutation_{—}3, L128G; site-directed mutation_{—}4, L132G.

Morphological Observations. Overexpression lines for each of four different mutagenized variants of G1792 have now been generated. The first of these sets of lines (G1792(E119V)) showed similar phenotype to G1792 overexpression; dark shiny curled leaves and dwarfing. Lines for the other variants, though, show much less severe dwarfing and lacked the glossy appearance. Instead, these other sets of lines had a rather dull silvery coloration, which perhaps indicated a change in wax composition at the leaf surface.

(1) Lines 961-980 (containing 35S::G1792(E119V)): all were markedly small with narrow, dark green, shiny leaves. The following lines were also very slow developing and bolted later than wild-type: 961, 965, 966, 968, 970, 971, 975, 977, 979, and 980.

(2) Lines 981-997 (containing 35S::G1792(D124G)): all were slightly small at early stages. Later, the lines were of wild-type size, but some (#982, 983, 984, 987, 991, 992, 993) had leaves that were flat, dull green in coloration, and showed slight serrations on the margins. At later stages, the leaves of these lines became rather contorted. Some of the lines were also late flowering: 982-984, 991-993 and 997.

(3) Lines 1001-1006 (containing 35S::G1792(L128G)): all were small at early stages. At later stages 4/6 of these lines (1003-1006) developed rather flat leaves that had a grayish silvery appearance. These lines were also slightly late flowering and showed a reduction in apical dominance. #1002 was tiny and #1001 appeared wild type at later stages.

(4) Lines 1021-1024 (containing 35S::G1792(L132G)): all were small at early stages. Later the plants were dull in coloration and developed dull, flat leaves with slightly serrated margins. A reduction in apical dominance was also apparent at late stages. #1022 was small, bushy, and had curled leaves versus wild-type.

Physiology (Plate assays) Results. In tests performed thus far, several of the lines harboring mutagenized variants of G1792 were more tolerant, relative to controls, in plate-based abiotic stress assays. Lines overexpressing site-directed mutation (2) had a higher rate of germination than controls under low nitrogen and high sucrose conditions (4 of 10 lines in this C/N sensing assay), less anthocyanin on basal media minus nitrogen plus 3% sucrose and 1 mM glutamine (6 of 10 lines in this second C/N sensing assay), and more root mass on a low nitrogen root growth assay (5 of 10 lines in this nutrient limitation assay).

Physiology (Soil Drought-Clay Pot) Summary. Two lines of mutagenized variants of G1792 were tested in the soil drought screen and showed a significantly better performance than wild-type controls.

TABLE 84
G1792 Point mutation drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 982 | Site Dir | 1.5 | 1.4 | 0.57 | 0.35 | 0.32 | 0.61 |
| | Mut 2 | | | | | | |
| 982 | Site Dir | 2.2 | 1.0 | 0.0018* | 0.41 | 0.31 | 0.11* |
| | Mut 2 | | | | | | |
| 986 | Site Dir | 3.2 | 2.3 | 0.092* | 0.76 | 0.62 | 0.015* |
| | Mut 2 | | | | | | |
| 986 | Site Dir | 2.4 | 1.9 | 0.21 | 0.54 | 0.48 | 0.34 |
| | Mut 2 | | | | | | |

Site Dir Mut 2 = Site directed mutation (2) project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Overexpression lines for each of these mutagenized variants of G1792 have now been generated. The first of these sets of lines (G1792(E119V)) showed similar phenotype to G1792 overexpression: dark, shiny, curled leaves and dwarfing. Lines for the other variants, though, show much less severe dwarfing and lacked the glossy appearance. Instead, these other sets of lines had a rather dull silvery coloration, which perhaps indicated a change in wax composition at the leaf surface. A similar phenotype was observed in a minority of lines for the G1792 deletion variant in which the EDLL domain was deleted, suggesting that this may represent a dominant negative phenotype.

Two different lines ectopically expressing site-directed mutagenesis #2 were more tolerant to drought than controls.

Potential Applications. Lines overexpressing G1792 with site-directed mutation (2) are likely to have better quality and greater yield in low nitrogen conditions or in drought conditions.

G1791 (SEQ ID NO: 229 and 230; Arabidopsis thaliana)—Vascular-Specific SUC2

Background. G1791 is a paralog of G1792. We have named G1791 TDR2 (Transcriptional regulator of the Defense Response 2).

The aim of this project was to determine whether expression of G1791 from a SUC2 promoter, which predominantly drives expression in a vascular specific pattern, is sufficient to confer pathogen resistance or drought tolerance, and whether expression of G1791 in vascular tissue causes any of the negative side effects of G1791 expression (stunting, delayed development and flowering).

Morphological Observations. Twenty SUC2_line12::G1791 lines have been isolated (741-761). Lines 748 and 757 died early in development. Lines 741-747 and 755 were small (20% control size). Lines 741, 742, 744, 745 and 755 were dark green and shiny. All lines were generally late flowering compared to control plants.

Physiology (Plate assays) Results. In tests performed thus far, several of the lines harboring mutagenized variants of G1791 under the regulator control of the SUC2 promoter showed altered C/N sensing. Three of 10 lines had a higher rate of germination than controls under low nitrogen and high sucrose conditions, and seedling of 5 of 10 lines had less anthocyanin on basal media minus nitrogen plus 3% sucrose and 1 mM glutamine.

Discussion. A set of two-component SUC2::G1791 lines has been isolated. A number of these lines were significantly smaller than controls, and some were dark green and shiny. All lines were late flowering. These phenotypes are similar to those caused by constitutive overexpression of members of the G1792 clade, although much less severe than seen in 35S::G1791 plants. These results suggest that expression of G1791 in vascular tissue may contribute to the deleterious effects seen in 35S::G1791 plants.

Potential applications. Based on the data so far, expression of G1791 in a vascular pattern may be used to modify developmental traits such as flowering time. These plants may also perform better under conditions of nitrogen limitation.

G1791 (SEQ ID NO: 229 and 230; Arabidopsis thaliana)—Epidermal-Specific LTP1

Background. The aim of this project was to determine whether expression of G1791 from an LTP1 promoter (which predominantly drives expression in the shoot epidermis and vascular tissue) is sufficient to confer pathogen resistance or drought tolerance, and whether expression of G1791 in epidermal and vascular tissue causes any of the negative side effects of G1791 expression (stunting, delayed development and flowering).

Morphological Observations. Thirty-two LTP1::G1791 lines were isolated (lines 321-332 and 381-400). No consistent differences to controls were observed.

Disease Summary: Five LTP1::G1791 two-component lines were tested in Sclerotinia and Botrytis plate assays, Two of these lines showed moderate resistance to Botrytis; the disease response to Sclerotinia was not affected.

N.B. TDR2 (Transcriptional regulator of the Defense Response) is the gene name for G1791. The control used in this experiment was the LTP1 promoter background supertransformed with a GUS target gene.

Discussion. LTP1::G1791 lines did not show any significant morphological differences to wild-type controls. Five LTP1::G1791 lines were analyzed for disease resistance: two of these lines showed enhanced resistance to Botrytis, but none to Sclerotinia. These lines have not yet been assayed for powdery mildew resistance; these assays will be of interest because powdery mildew infects only epidermal cells. Ten lines were tested in plate-based abiotic stress assays; no consistent enhanced tolerance was seen in any assay.

Potential applications. The results obtained to date indicate that expression of G1791 under an LTP1 promoter may enhance disease resistance (while minimizing deleterious growth effects seen with constitutive expression).

G1791 (SEQ ID NO: 229 and 230; Arabidopsis thaliana)—Epidermal-Specific CUT1

Background. The aim of this project was to determine whether expression of G1791 from a CUT1 promoter (which predominantly drives expression in a shoot epidermal pattern, with high levels of expression in guard cells) is sufficient to confer pathogen resistance or drought tolerance, and whether expression of G1791 in epidermal tissue causes any of the negative side effects of G1791 expression (stunting, delayed development and flowering).

Morphological Observations. Twenty six CUT1::G1791 lines were isolated in two batches (581-591 and 661-675). CUT1::G1791 plants were typically smaller than controls early in development, and approximately one-third of the lines were slightly late developing compared to controls. No other differences were evident.

Disease Summary. Six of eight CUT1::G1791 two-component lines showed moderate resistance to Sclerotinia in plate assays. The disease response to Botrytis was not affected.

N.B. TDR2 (Transcriptional regulator of the Defense Response) is the gene name for G1791. The control used in this experiment was the CUT1 promoter background supertransformed with a GUS target gene.

Physiology (Plate assays) Results. Three out of ten CUT1::G1791 lines were more tolerant to a severe dehydration stress compared with wild-type seedlings.

Discussion. CUT1::G1791 plants were typically smaller than controls early in development, and approximately one-third of the lines were slightly late developing compared to controls. Three out of ten CUT1::G1791 lines were more tolerant to a severe dehydration stress than controls in a plate assay.

Potential applications. The results obtained to date indicate that expression of G1791 under a CUT1 promoter may enhance abiotic stress tolerance while decreasing the negative side effects of constitutive G1791 expression.

G1791 (SEQ ID NO: 229 and 230; Arabidopsis thaliana)—Leaf-Specific RBCS3

Background. The aim of this project was to determine whether expression of G1791 from an RBCS3 promoter, which drives expression in photosynthetic tissue, is sufficient to confer pathogen resistance, and whether restricting expression of G1791 to photosynthetic tissue can alleviate the negative side effects of G1791 expression (stunting, delayed development and flowering).

Morphological Observations. Twenty RBCS3::G1791 lines have been isolated (361-380). Overall they were not consistently different from control lines. Lines 367, 368 and 373 were slightly smaller than controls and lines 362, 363, 367 and 368 were slightly late flowering.

Physiology (Plate assays) Results. Five out of ten RBCS3::G1791 lines were insensitive to ABA in a germination assay. Three of these lines were more tolerant than controls to cold in a chilling growth assay.

Discussion. In general, the two-component RBCS3::G1791 lines examined showed no consistent morphological differences from controls, although four lines were slightly late flowering. Five RBCS3::G1791 lines were analyzed for Botrytis and Sclerotinia resistance through the SBIR grant, and no consistent disease resistance phenotype was found. The lines were tested in plate based assays and showed a better performance than controls in ABA germination and cold growth assays.

Potential applications. The RBCS3::G1791 combination may be used to confer tolerance to abiotic stress such as cold and drought (while minimizing deleterious growth effects seen with constitutive expression).

G1791 (SEQ ID NO: 229 and 230; Arabidopsis thaliana)—GR Fusion (Dex Inducible)

Background. Since G1791 produces severe dwarfing when overexpressed, the dexamethasone-inducible lines the lines were generated to allow us to test lines in disease assays with simultaneous dexamethasone applications.

Morphological Observations. The following sets of dexamethasone inducible lines have been generated:

Two-Component Lines:

Lines 341-360:

All T1 lines appeared wild type in the absence of dex, except for 342 which was dark green and dwarfed.

Direct Fusion Lines:

Lines 641-660 (containing a 35S::G1791-GR fusion):

Many of these T1 lines were dwarfed and showed rather long petioles in the absence of dex. (#641, 642, 644, 646, 647, 649, 654 had long petioles and 643, 645, 650-653, 655, 656-660 were small.) Some alterations in flowering time were observed: 642, 644, 648, 649, 651, 652, 654, 657, 660 were early developing, whereas #653 and 656 were slightly late flowering. #643 was tiny and dark green. In other respects, the lines showed no consistent differences to controls.

Lines 710-720 (Containing a 35S::GR-G1791 Fusion):

These T1 lines appeared wild type except for 702, 712-714, and 720 which were slightly early flowering.

Disease Summary. Five two-component dexamethasone (dex)-inducible G1791 lines were tested in Sclerotinia and Botrytis plate assays. Three of these lines showed moderate to strong resistance to both pathogens when grown on plates containing dex. Of these three lines, two (351 and 353) did not show enhanced resistance when grown on regular (minus dex) plates, as expected. Line 350 did have increased resistance to both pathogens on regular plates, however the degree of resistance was lower than on dex-containing plates. This apparent “leakiness” of the transgene in line 350 could be attributed to a position effect of the insertion site of the target construct.

N.B. TDR2 (Transcriptional regulator of the Defense Response) is the gene name for G1791. The control used in this experiment was the dex-inducible promoter background supertransformed with a GUS target gene.

Discussion. A set of two-component lines was obtained. In the absence of dexamethasone, these plants appeared wild type. Five of these lines were tested in disease assays, and three lines showed moderate to strong resistance to Sclerotinia and Botrytis when pre-treated with dexamethasone.

N-terminal and C-terminal direct GR fusions to G1791 were also obtained. A relatively high number of the 35S::G1791-GR lines showed dwarfing and long petioles in the absence of Dex, perhaps indicating that these fusions are not confined to the cytoplasm, or that they exert negative effects in the cytoplasm. The 35S::GR-G1791 lines appeared wild type.

Potential applications. The results obtained to date indicate that expression of G1791 under an inducible promoter may be sufficient to provide enhanced disease resistance.

G1795 (SEQ ID NO: 223 and 224; Arabidopsis thaliana)—Vascular-Specific SUC2

Background. G1795 is a paralog of G1792. Analysis of G1795 under alternative promoters for disease resistance was initiated, and it was found in this work that expression of G1795 under a green tissue or inducible promoter can confer Botrytis and Sclerotinia resistance. We have named G1795 TDR3 (Transcriptional regulator of the Defense Response 3).

The aim of this project was to determine whether expression of G1795 from a SUC2 promoter, which predominantly drives expression in a vascular specific pattern, is sufficient to confer drought tolerance or pathogen resistance, and whether expression of G1795 in vascular tissue causes any of the negative side effects of G1795 expression (stunting, delayed development and sterility).

Morphological Observations. Twenty SUC2::G1795 (2-component) lines were isolated (481-500). These lines were consistently small and late flowering, and leaves were consistently shiny and narrow. The exception was line 482 which appeared wild type.

Physiology (Plate assays) Results. Six out of ten SUC2::G1795 lines were darker green in a germination assay in the presence of mannitol. Five of these lines were also insensitive to ABA in another germination assay. Three of these lines also had more root growth in a growth assay under limited nitrogen. Four lines were also more tolerate to a severe dehydration stress compared to wild-type seedlings.

Discussion. SUC2::G1795 plants were consistently small and late flowering, with shiny, narrow leaves. These phenotypes are consistent with those seen with overexpression of other members of the G1792 clade. However, these effects were not as severe as those seen in constitutively-overexpressing 35S::G1795 plants. Several SUC2::G1795 lines were insensitive to ABA, tolerant to mannitol in germination assays, showed more root growth than controls on limited nitrogen, and showed enhanced tolerance to severe dehydration. Therefore, it seems that expression of G1795 in vascular tissue can confer abiotic stress tolerance, but is associated with some lesser degree of deleterious morphological effects when expression is regulated by a vascular, rather than constitutive, promoter.

Potential applications. The results obtained to date indicate that expression of G1795 under a vascular promoter may enhance abiotic stress tolerance, while reducing the deleterious side effects conferred by constitutive G1795 expression. However, it should be noted that substantial morphological off-types were still seen in SUC2::G1795 lines. In addition to stress tolerance, this gene might also be used to influence developmental traits such as flowering time, leaf shape, and surface wax accumulation.

G1795 (SEQ ID NO: 223 and 224; Arabidopsis thaliana)—Epidermal-Specific CUT1

Background. The aim of this project was to determine whether expression of G1795 from an CUT1 promoter (which predominantly drives expression in vascular tissue) is sufficient to confer pathogen resistance, and whether expression of G1795 in epidermal and/or vascular tissue causes any of the negative side effects of G1795 expression (stunting, delayed development and sterility).

Morphological Observations. Twenty-one CUT1::G1795 lines were isolated. These lines were generally small, dark green and the majority were also late flowering. They also typically had shiny leaves. The degree of severity of these traits was variable.

Disease Summary. Eight CUT1::G1795 lines were tested in a Sclerotinia plate assay. Three lines were more resistant than controls to this pathogen.

G1795 (SEQ ID NO: 223 and 224; Arabidopsis thaliana)—Epidermal-Specific LTP1

Background. The aim of this project was to determine whether expression of G1795 from an LTP1 promoter (which predominantly drives expression in the shoot epidermis and vascular tissue) is sufficient to confer pathogen resistance or drought tolerance, and whether expression of G1795 in epidermal and/or vascular tissue causes any of the negative side effects of G1795 expression (stunting, delayed development and sterility).

Morphological Observations. Twenty LTP1::G1795 lines were isolated (301-320). All lines were small and dark, starting during the vegetative phase of development. Lines 301-303 and 306 were late flowering. Lines 301, 302, 308, 314-316 and 318-320 showed early senescence. Contrary to typical Arabidopsis development, late flowering and early senescence were seen in some of the same LTP1::G1795 plants.

Disease Summary. Eight LTP1::G1795 two-component lines were tested in a Sclerotinia plate assay and five lines were tested in a Botrytis plate assay. Five lines showed moderate to strong resistance to Sclerotinia, and four lines showed enhanced resistance to Botrytis.

Three lines that showed Sclerotinia resistance were tested in a soil-based assay. All three lines showed better survival than controls after 4 days in one run of this assay.

N.B. TDR3 (Transcriptional regulator of the Defense Response) is the gene name for G1795. The control used in this experiment was the LTP1 promoter background supertransformed with a GUS target gene.

Discussion. All LTP1::G1795 lines analyzed were small and dark green. A few lines were late flowering, and several showed early senescence, including some of the late flowering plants. The small, dark green, and late flowering phenotypes are typical of members of the G1792 clade (though much less severe than seen in 35S::G1795 plants), but early senescence (indicating that G1795 might regulate senescence pathways) has not been noted in this study group before.

Five LTP1::G1795 two-component lines were tested in Sclerotinia and Botrytis plate assays. Three of these lines showed moderate to strong resistance to Sclerotinia and Botrytis, and a fourth line displayed enhanced resistance only to Botrytis. These lines have not yet been tested in powdery mildew assays. In contrast, LTP1::G1795 showed a wild-type response in plate-based abiotic stress assays.

Potential applications. The results obtained to date indicate that expression of G1795 under the LTP1 promoter may enhance disease resistance. This promoter yielded less severe, but did not completely remove, the morphological and developmental adverse effects associated with constitutive G1795 overexpression.

G1795 (SEQ ID NO: 223 and 224; Arabidopsis thaliana)—Leaf-Specific RBCS3

Background: The purpose of this program was to determine if G1795 under alternative under a green tissue or inducible promoter can confer Botrytis and Sclerotinia, or other disease resistance.

Morphological Observations. Eighteen RBCS3::G1795 lines were isolated (341-343 and 381-395). All lines were small at the rosette stage of development, and had dark green leaves. All lines flowered late.

Disease Summary. Eight RBCS3:G1795 two-component lines were tested in the Sclerotinia plate assay, and six of these lines were also tested in a Botrytis plate assay. Five of these lines displayed moderate to strong enhanced resistance to both Sclerotinia and Botrytis.

Three lines that showed resistance to Sclerotinia in a plate assay were tested in a Sclerotinia soil assay. All three lines showed significantly better plant survival in one run of the assay.

N.B. TDR3 (Transcriptional regulator of the Defense Response) is the gene name for G1795. The control used in this experiment was the RBCS3 promoter background supertransformed with a GUS target gene.

Discussion. All RBCS3::G1795 lines were small with dark green leaves at the rosette stage of development, and flowered late. However, these phenotypes were much less severe than those seen in 35S::G1795 lines. Five RBCS3::G1795 two-component lines were tested in Sclerotinia and Botrytis plate assays. Four of these lines displayed moderate to strong enhanced resistance to both Sclerotinia and Botrytis. However, RBCS3::G1795 lines did not perform significantly differently from wild-type controls in plate-based abiotic stress assays.

Potential applications. The results obtained to date indicate that expression of G1795 under an RBCS3 promoter may enhance disease resistance. An RBCS3 expression pattern did not appear to achieve this while completely eliminating morphological off-types.

G30 (SEQ ID NO: 225 and 226; Arabidopsis thaliana)—Vascular SUC2

Background. G30 is a paralog of G1792. We have named G30 TDR4 (Transcriptional regulator of the Defense Response 4).

The aim of this project was to determine whether expression of G30 from a SUC2 promoter, which predominantly drives expression in a vascular specific pattern, is sufficient to confer pathogen resistance or drought tolerance, and whether expression of G30 in vascular tissue causes any of the negative side effects of G30 overexpression (stunting, delayed development, and sterility).

Morphological Observations. Twenty SUC2::G30 lines were isolated (541-560). All lines had leaves that were dark, shiny and small. Leaves were also curly vs. control. All lines also were late flowering, with the exception of line 559 which was small and dark but developed normally vs. control. After bolting, lines 543, 545, 547, 550, 552, 553, 555 and 560 were small and spindly. Line 551 had large, wide, curly leaves vs. control.

Physiology Results. Five of 10 lines were more tolerant to mannitol, 7 of 10 lines were more tolerant to cold during germination, and 3 of 10 lines were more tolerant to desiccation in a plate-based assay than controls. Seedlings of 8 of 10 lines had less anthocyanin on basal media minus nitrogen plus 3% sucrose and 1 mM glutamine in this second C/N sensing assay.

Discussion. All SUC2::G30 lines analyzed were small with dark, shiny, and curly leaves. All lines but one were late flowering. The small, dark green, and late flowering phenotypes are typical of members of the G1792 clade, though much less severe than seen in 35S::G30 plants.

Potential Applications. Plants overexpressing G30 under the regulator control of vascular promoters such as SUC2 may have greater quality and yield than non-transformed plants under conditions of nutrient (e.g., nitrogen) limitation.

G30 (SEQ ID NO: 225 and 226; Arabidopsis thaliana)—Leaf RBCS3

Background. We have named G30 TDR4 (Transcriptional regulator of the Defense Response 4). The aim of this project was to determine whether expression of G30 from an RBCS3 promoter, which drives expression in photosynthetic tissue, is sufficient to confer pathogen resistance or drought tolerance, and whether restricting the expression of G30 to photosynthetic tissue alleviates the negative side effects of G30 overexpression (stunting, delayed development and sterility).

Morphological Observations. Twenty RBCS3::G30 lines have been isolated (Lines 361-380). All of these lines were at least marginally late flowering, and had dark green/slightly wrinkled leaves. Lines 361, 368, 369 and 376 were the latest flowering lines, and lines 368 and 375 were the smallest lines (30% of controls).

Five RBCS3::G30 two-component lines were tested in Sclerotinia and Botrytis plate assays. Three of these lines (370, 374, and 377) showed moderate to strong resistance to Sclerotinia and Botrytis. Line 362 only provided enhanced Botrytis resistance.

N.B. TDR4 (Transcriptional regulator of the Defense Response) is the gene name for G30. The control used in this experiment was the RBCS3 promoter background line supertransformed with a GUS target gene.

Discussion. All the RBCS3::G30 lines characterized were at least marginally late flowering, and had dark green/slightly wrinkled leaves. The small, dark green, and late flowering phenotypes are typical of members of the G1792 clade, though much less severe than seen in 35S::G30 plants. Five RBCS3::G30 two-component lines were tested in Sclerotinia and Botrytis plate assays. Three of these lines showed moderate to strong resistance to Sclerotinia and Botrytis, while a fourth line only showed enhanced Botrytis resistance. These lines have not yet been tested in powdery mildew assays. In contrast, RBCS3::G30 lines did not perform significantly differently from wild-type controls in plate-based abiotic stress assays.

Potential applications. The results obtained to date indicate that expression of G30 under a green tissue promoter may enhance disease resistance. However, it should be noted that this promoter still resulted in developmental off-types.

G30 (SEQ ID NO: 225 and 226; Arabidopsis thaliana)—Epidermal LTP1

Background. The aim of this project was to determine whether expression of G30 from an LTP1 promoter (which predominantly drives expression in the shoot epidermis and vascular tissue) is sufficient to confer pathogen resistance or drought tolerance, and whether restricting expression of G30 to epidermal tissue alleviates any of the negative side effects of G30 expression (stunting, delayed development and sterility).

Morphological Observations. Thirteen LTP1::G30 lines were isolated from two batches of T1 plants (341-346 and 381-387. All plants were small in size and dark in color, with curling upright leaves compared to controls. All lines also flowered and developed late.

Physiology (Plate assays) Results. Three out of ten lines were more tolerant to low nitrogen in a growth assay than wild-type control seedlings. Three other lines did not accumulate anthocyanins in a cold germination assay.

Discussion. All LTP1::G30 plants analyzed were small in size and dark in color, with curling upright leaves compared to controls. All lines also flowered and developed late. The small, dark green, and late flowering phenotypes are typical of members of the G1792 clade, though much less severe than seen in 35S::G30 plants.

Three out of ten LTP1::G30 lines showed more tolerance to a low nitrogen growth assay than wild-type control seedlings. Three other lines did not accumulate anthocyanins in a cold germination assay, indicating that these lines may be more tolerant to cold germination. No soil drought studies have been performed on these lines. Only one out of five lines tested showed altered performance in disease assays.

Potential applications. The results obtained to date indicate that expression of G30 under the LTP1 promoter may confer enhanced tolerance to abiotic stress. However, LTP1 lines still showed developmental off-types. There is not yet compelling evidence that the LTP1 promoter is a suitable promoter to use in combination with G30 to produce enhanced disease resistance.

G30 (SEQ ID NO: 225 and 226; Arabidopsis thaliana)—Emergent Leaf primordia AS1

Background. The aim of this project was to determine whether expression of G30 from an AS1 promoter, which drives expression in emergent leaf primordia, causes any of the negative side effects of G30 overexpression (stunting, delayed development and sterility).

Morphological Observations. Twenty AS1::G30 lines have been isolated (741-760). Seedlings typically had long hypocotyls and upward pointing cotyledons. Lines 741, 747, 752, 758 and 759 were slightly darker green prior to flowering, and all lines had upward pointing rosette leaves.

At the time of bolting, lines 741-744, 746-749, 752, 753 and 757-759 were small (30-60% control size). Lines 744, 746, 748-750, 752, 753 and 757-759 were dark green in coloration. Lines 744, 746, 749, 750 and 757 are late developing vs. control. These phenotypes continued to be evident throughout later development.

Discussion. AS1::G30 seedlings typically had long hypocotyls and upward pointing cotyledons. All lines also had upward pointing rosette leaves, indicating that the gene influences light-regulated development. At the time of bolting, most lines were small and dark green, and several were late flowering. The small, dark green, and late flowering phenotypes are typical of members of the G1792 clade, though much less severe than those seen in 35S::G30 plants.

Potential Applications. Plants overexpressing G30 under the regulatory control of emergent leaf primordia promoters such as AS1 have morphological and developmental effects that are much less severe than plants constitutively expressing this gene, and thus this combination may be used to manipulate photosynthetic rate, light-regulated development and flowering time without the severe effects seen in the latter plants.

G30 (SEQ ID NO: 225 and 226; Arabidopsis thaliana)—GR Fusion (Dex Inducible)

Background. Since G30 produces severe dwarfing when overexpressed, dexamethasone-inducible lines were generated to allow us to test lines in disease assays after dexamethasone application.

Morphological Observations. The following sets of dexamethasone inducible lines have been generated:

Two-Component Lines:

Lines 321-340:

All were dwarfed (except #339) in the T1 generation and showed a phenotype similar to, but less severe than 35S::G30 lines. These phenotypes were seen in the absence of dexamethasone suggesting that the lines were “leaky.”

Interestingly, though, in later generations, three of these lines were morphologically examined, and appeared wild type in the absence of dexamethasone. Homozygous T3 generation populations have now been established for each of these lines (321, 322, 339, see table below).

Direct Fusion lines:

Lines 621-640 (containing a 35S::G30-GR Fusion):

The majority of these use T1 lines appeared wild type in the absence of dexamethasone except for 622, 625, 626, 637 which were dwarfed with shiny leaves. Some of the lines were slightly early flowering (#631, 632, 634, 636, 638-640).

Lines 621-640 (containing a 35S::GR-G30 fusion):

These lines were of wild-type size, but a substantial number of the lines were early flowering in the absence of dexamethasone: 643, 644, 646, 647, 650-656, 658-660.

Disease Summary. Five two-component dexamethasone (dex)-inducible G30 lines were tested in Sclerotinia and Botrytis plate assays. Three of these lines (321, 322, and 339) showed strong resistance to Sclerotinia and Botrytis when grown on plates containing dex. Line 321 also provided enhanced pathogen resistance on regular (minus dex) plates, however the degree of resistance was lower than on dex-containing plates. This apparent “leakiness” of the transgene in line 321 could be attributed to a position effect of the insertion site of the target construct.

N.B. TDR4 (Transcriptional regulator of the Defense Response) is the gene name for G30. The control used in this experiment was the dex-inducible promoter background supertransformed with a GUS target gene.

Physiology Results. Dexamethasone-inducible G30 lines were more tolerant to abiotic stresses. These included salt (5 of 10 lines), mannitol (hyperosmotic stress; 7/10), ABA (5 of 10 lines), and heat during germination (6 of 10 lines).

Discussion. Dexamethasone (dex) inducible G30 overexpression lines have been established using the two-component system. All but one line were dwarfed in the T1 generation and showed a phenotype similar to, but less severe than 35S::G30 lines, suggesting that G30 expression was not tightly off in these lines. It impossible that this is due to regulatory sequences present in the UTR or coding sequence in the opLexA::G30 construct, although relatively little 5′ or 3′ UTR is present. However, in later generations, such dwarfing effects were much less apparent.

Five two-component dexamethasone (dex)-inducible G30 lines were tested in Sclerotinia and Botrytis plate assays. Three of these lines showed strong resistance to Sclerotinia and Botrytis when grown on plates containing dex. One line also provided enhanced pathogen resistance on regular (minus dex) plates, however the degree of resistance was lower than on dex-containing plates. This apparent “leakiness” of the transgene is consistent with the morphological phenotypes observed in the absence of dex.

N-terminal and C-terminal direct GR fusions to G30 have also been created. The majority of lines produced for each construct were of wild-type size, but some were early flowering. No disease assays have been performed on these lines.

Potential applications. The results obtained to date indicate that expression of G30 under an inducible promoter can confer disease resistance and tolerance to various abiotic stresses without the plants developing severe off-types (developmental and/or morphological defects).

G1266 (SEQ ID NO: 253 and 254; Arabidopsis thaliana)—Constitutive 35S

Background. G1266 corresponds to ERF1 (as opposed to AtERF1, which corresponds to G28), a gene with known functions in ethylene/jasmonate and disease signal transduction (Solano et al., 1998; Lorenzo et al., 2003). ERF1 overexpressing lines have been shown to have increased resistance to multiple pathogens (Berrocal-Lobo et al., 2002; Berrocal-Lobo and Molina, 2004), and in the genomics program we observed enhanced resistance to E. orontii. ERF1 (and potentially other ERF genes) are thought to function antagonistically to AtMYC2 to balance the ethylene/jasmonate-dependent pathogen resistance pathway against the jasmonate-dependent wound response pathway (Boter et al., 2004; Lorenzo et al., 2004). G1266 was included in this study because it is related to G1792, and because we wanted to compare the strength of the disease resistance phenotype induced by G1266 to that of other genes in the G1792 and G28 study groups.

Morphological Observations. G1266 produced severe dwarfing when overexpressed. Almost all of the lines in each of two different batches were very small, slow developing, and exhibited narrow, dark, rather shiny leaves. A total of thirty new lines have been obtained: 301-320 and 321-330.

Physiology (Plate assays) Results. Five out of ten 35S::G1266 lines were insensitive to ABA in a germination assay. Two of these lines were also tolerant to NaCl and mannitol in a germination assay. Two other lines were more tolerant to cold in another germination assay.

Disease Summary. In our earlier genomics program, G1266 overexpressing lines were found to be more tolerant to the fungal pathogen Erysiphe orontii but were wild type in their response to Sclerotinia, Botrytis, and Fusarium. Eight new 35S::G1266 lines were tested by Sclerotinia and Botrytis plate assays; several of these lines displayed altered pathogen response phenotypes. Three of these lines showed enhanced resistance to Botrytis, one line showed enhanced resistance to Sclerotinia, and one line showed enhanced resistance to both pathogens. Lines 304 and 307 displayed enhanced disease symptoms in response to Botrytis.

Discussion. 35S::G1266 plants were severely dwarfed, dark green, and late flowering, with narrow, shiny leaves. These phenotypes are consistent with those observed for plants overexpressing members of the G1792 clade.

As members of the G1792 study group, 35S::G1266 lines were analyzed in both the drought-related and disease-related screens. Five 35S::G1266 lines showed insensitivity to ABA in a germination assay. Four of these lines also produced hits in sodium chloride, mannitol, or cold germination assays. Three lines were tested in the clay pot drought assay, but no significant difference from wild-type plants was observed.

Four lines were more resistant to Botrytis in a plate assay, and two lines showed more resistance in a Sclerotinia assay. There was some overlap between lines that performed well in the plate-based abiotic stress and disease assays; three lines that were hits in the abiotic stress assays also hit in one or more disease assays.

It is possible that the ABA insensitivity and disease resistance phenotypes of 35S::G1266 plants are linked: there are known antagonistic interactions between the ethylene/jasmonate and ABA signal transduction pathways (Anderson et al., 2004). ABA suppresses basal JA/ethylene pathway signaling, ethylene insensitive mutants show increased expression of ABA-dependent reporter genes, and some ABA-insensitive mutants are more resistant to necrotrophic pathogens (Anderson et al., 2004). However, the converse (that increased JA/ethylene signaling mediated at the level of AtERF1 can suppress ABA signaling) seems plausible, but has not to our knowledge been directly demonstrated.

Potential applications. Based on the results obtained so far, G1266 may be used to increase abiotic stress tolerance or disease resistance, or modify plant development and flowering time.

G1752 (SEQ ID NO: 401 and 402; Arabidopsis thaliana)—Constitutive 35S

Background. G1752 was included in this research program because of its close relationship to G1792. G1752 is a member of an Arabidopsis sub-clade that includes G1266. The aim of this project was to determine whether overexpression of G1752 in Arabidopsis produces comparable effects to those of G1792 overexpression.

Morphological Observations. G1752 overexpressors tended to be a slightly darker green than control seedlings. Several lines were chlorotic, and had less root growth than wild-type controls. G1752 produced severe dwarfing when overexpressed. Almost all of the lines in each of two different batches were very small, slow developing, and exhibited narrow dark leaves. The most severely affected individual died at early stages.

Physiology (Plate assays) Results. Three out of seven 35S::G1752 lines were tolerant to mannitol in a germination assay.

Discussion. G1752 was highly deleterious when expressed under the 35S promoter. Most lines were severely dwarfed and slow developing, with dark green narrow leaves. These phenotypes are consistent with those observed for G1752 in the genomics program, and for some other members of the G1792 study group.

Potential applications. Based on the results obtained so far, G1752 may be applied to increase hyperosmotic stress tolerance if expressed under an appropriate promoter.

G3380 (SEQ ID NO: 249 and 250; Oryza sativa)—Constitutive 35S

Background. G3380 is a closely-related rice homolog of G1792. The aim of this study was to determine whether overexpression of G3380 can confer abiotic stress tolerance and disease resistance similarly to G1792.

Morphological Observations. The overexpression of G3380 consistently induced mild dwarfing, and a slight delay in flowering. Plants were isolated in two different batches, and in the first batch, the Ti seedlings were small and vitrified. A total of twenty-four 35S::G3380 lines were isolated: 301-311 and 321-333.

Physiology (Plate assays) Results. Five out of 10 35S::G3380 lines were more tolerant to mannitol in a germination assay than control seedlings. Six of 10 lines were also more tolerant than controls when germinated in the presence of mannitol. Three of 10 lines also showed tolerance to cold than controls in a growth assay.

Physiology (Soil Drought-Clay Pot) Summary. Three independent 35S::G3515 lines were tested in a single run of the soil drought screen and all three showed a significantly better performance than wild-type controls.

TABLE 85
35S::G3380 drought assay results.

| | | Mean | Mean | p-value for | | Mean | p-value for |
|---|---|---|---|---|---|---|---|
| | Project | drought | drought score | drought score | Mean survival | survival | difference in |
| Line | Type | score line | control | difference | for line | for control | survival |
| 301 | DPF | 2.3 | 1.4 | 0.023* | 0.41 | 0.40 | 0.90 |
| 301 | DPF | 1.5 | 1.3 | 0.59 | 0.27 | 0.22 | 0.37 |
| 307 | DPF | 3.0 | 2.0 | 0.12 | 0.54 | 0.42 | 0.043* |
| 307 | DPF | 1.9 | 1.0 | 0.00053* | 0.37 | 0.20 | 0.0022* |
| 322 | DPF | 3.0 | 1.5 | 0.00086* | 0.65 | 0.33 | 0.00000013* |
| 322 | DPF | 2.8 | 1.1 | 0.0015* | 0.57 | 0.29 | 0.0000027* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. The overexpression of G3380 consistently induced mild dwarfing, and a slight delay in flowering. Overexpression of members of the G1792 clade tends to produce small, dark green, and late flowering plants. However, this phenotype displayed by 35S::G3380 plants is milder than for most members of the clade, and yet these overexpressors performed well in hyperosmotic, cold, and drought stress assays.

Potential applications. G3380 may be used to enhance tolerance to drought-related stresses and cold.

G3381 (SEQ ID NO: 233 and 234; Oryza sativa)—Constitutive 35S

Background. G3381 is a closely-related rice homolog of G1792. The aim of this study was to determine whether overexpression of G3381 can confer abiotic stress and disease resistance similarly to G1792.

Morphological Observations. To date, a total of six 35S::G3381 lines have been obtained (Lines 301-306), however, only one batch of plants has been evaluated. Line 301 appeared wild type, but in all other lines, the overexpression of G3381 consistently induced dwarfing, spindly leaves and dark green coloration.

Physiology (Plate assays) Results. Three out of four 35S::G3381 lines performed better than wild-type seedlings in a germination assay under cold conditions. Two of these lines also did well when germinated in the presence of mannitol. Some lines also showed tolerance to NaCl, ABA, heat, C/N sensing, and growth under low nitrogen.

Disease Summary. Five 35S::G3381 lines were tested by Sclerotinia plate assay. Lines 301, 302, and 306 displayed enhanced resistance to Sclerotinia. Three of the five lines were also tested by Botrytis plate assay. The response to this pathogen was variable: one line showed an enhanced resistance to Botrytis while another line appeared to be slightly more susceptible.

Discussion. The overexpression of G3381 induced strong dwarfing and dark green coloration, phenotypes typical of overexpression of members of the G1792 clade. Only six 35S::G3381 lines have been recovered to date. (Because of the strong phenotype associated with G3381 overexpression, RBCS3::G3381 lines are also being created for evaluation.) A limited number of 35S::G3381 lines were available for testing in stress assays: one of three lines performed well in a soil drought assay and a better performance than controls was seen in plate-based mannitol and cold germination assays. Three of five lines also showed resistance to Sclerotinia in disease assays.

Potential applications. G3381 may be used to enhance tolerance to abiotic stresses such as drought and cold. The gene might also be applied to enhance disease resistance. However, given the off-types seen with overexpression, G3381 would likely need to be optimized with tissue specific or inducible promoters.

G3383 (SEQ ID NO: 227 and 228; Oryza sativa)—Constitutive 35S

Background. G3383 is a closely-related rice homolog of G1792. The aim of this study was to determine whether overexpression of G3383 can confer abiotic stress tolerance and disease resistance similarly to G1792.

Morphological Observations. The overexpression of G3383 produced plants that were not consistently different from controls. However, Lines 303 and 307 were slightly pale and had small rosettes and spindly inflorescences. Line 304 showed reduced fertility. A total of seventeen new lines were obtained: 301-317.

Physiology (Plate assays) Results. 35S::G3383 lines have been analyzed in abiotic stress assays. Seven out of ten lines showed tolerance to cold temperatures in a growth assay. Four of these lines were also tolerant to mannitol in a germination assay. Three of the seven lines also performed better than wild-type control seedlings in a severe dehydration assay. Three lines also performed well in a cold germination assay.

Discussion. 35S::G3383 plants were not consistently different from controls in morphology. These plants produced hits in several abiotic stress assays: seven of ten lines were more tolerant to cold in a growth assay, three lines performed well in a cold germination assay, three lines showed more survival in a severe dehydration assay, and four lines showed more tolerance to germination on mannitol. No soil drought assays have been performed on these lines, and disease assays are still in progress.

Potential applications. The results obtained to date indicate that constitutive expression of G3383 may confer enhanced tolerance to abiotic stress. Importantly, unlike most of the genes from the G1792 study group, G3383 did not produce marked developmental off-types when overexpressed.

G3515 (SEQ ID NO: 237 and 238; Oryza sativa)—Constitutive 35S

Background. G3515 is a closely-related rice homolog of G1792. The aim of this study was to determine whether overexpression of G3515 can confer abiotic stress tolerance and disease resistance similarly to G1792.

Morphological Observations. The overexpression of G3515 did not induce a morphological phenotype distinct from that of the controls. Some size differences were noted early in development, but later all lines were similar in size to control plants. A total of twenty new lines have been obtained: 301-320.

Physiology (Plate assays Results. Five out of ten 35S::G3515 lines had more root growth (more root mass) and increased root hair density compared to wild-type control seedlings, when grown on plates in the absence of a stress treatment. This phenotype became stronger in some of the lines under low N conditions.

Physiology (Soil Drought-Clay Pot) Summary. Three independent 35S::G3515 lines were tested in a single run of the soil drought screen and all three showed a significantly better performance than wild-type controls.

TABLE 86
35S::G3515 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 310 | DPF | 0.67 | 0.33 | 0.45 | 0.19 | 0.032 | 0.00067* |
| 313 | DPF | 1.0 | 0.33 | 0.18 | 0.27 | 0.032 | 0.000015* |
| 319 | DPF | 1.5 | 0.33 | 0.039* | 0.35 | 0.032 | 0.00000063* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. 35S::G3515 plants were not consistently different from controls in morphology. Five out of ten lines showed more root growth in plate-based abiotic stress assays. Three independent 35S::G3515 lines were tested in a single run of the soil drought screen and all three of them showed a significantly better performance than controls. Disease assays are still in progress on these lines.

Potential applications. The results obtained to date indicate that constitutive expression of G3515 may confer enhanced tolerance to drought stress. The gene might also be used to manipulate root development. Importantly, unlike most of the genes from the G1792 study group, G3515 did not produce marked developmental off-types when overexpressed.

G3516 (SEQ ID NO: 239 and 240; Zea mays)—Constitutive 35S

Background. G3516 is a closely-related maize homolog of G1792. The aim of this study was to determine whether overexpression of G3516 can confer abiotic stress tolerance and disease resistance similarly to G1792.

Morphological Observations. Twenty 35S::G3516 lines have been isolated (Lines 301-320). Early in development these lines showed no differences compared to control plants. Later all lines showed serrated rosette leaves and many had reduced trichome density. Lines 304 and 305 had albino leaf sectors in adult plants. Late in development all lines were bushy, slightly small and spindly-leafed compared to controls.

Physiology (Plate assays) Results. 35S::G3516 lines accumulated less anthocyanins in a cold germination assay and in a germination assay designed to test C/N sensing. The overexpressors also accumulated less anthocyanin and had more root mass than controls on low nitrogen-containing medium. These results indicate that the overexpressors are more tolerant to cold and low nitrogen conditions than the wild-type controls.

Discussion. 35S::G3516 lines showed serrated rosette leaves, and many had reduced trichome density. Late in development, all lines were bushy and slightly small compared to controls. These lines accumulated less anthocyanins in a cold germination assay and in a germination assay designed to test C/N sensing. Disease assays on these lines are in progress.

Potential applications. The results obtained to date indicate that constitutive expression of G3516 may confer enhanced tolerance to abiotic stress, including cold and low nitrogen conditions.

G3517 (SEQ ID NO: 243 and 244; Zea mays)—Constitutive 35S

Background. G3517 is a closely-related maize homolog of G1792. The aim of this study was to determine whether overexpression of G3517 can confer abiotic stress tolerance and disease resistance similarly to G1792.

Morphological Observations. Twenty 35S::G3517 T1 lines were isolated (Lines 301-320). Considerable morphological variation was seen in these lines. Some of the lines were early flowering (301, 303, 305, 311, 312, 317, 320) and most of the lines showed dwarfing and rather curled leaves. The latter phenotype was particularly apparent in: 301-304, 309,310-312, 314-317.

Physiology (Plate assays) Results. Three out of ten lines performed better than wild-type seedlings in either a heat germination assay or under chilling conditions in a growth assay. Two lines were also more tolerant to severe dehydration.

Disease Results. Four of eight 35S::G3517 lines tested showed moderately to significantly greater resistance to Sclerotinia than wild type controls.

Discussion. 35S::G3517 T1 lines showed considerable variation in morphology and most were dwarfed. These lines showed enhanced tolerance to heat in a germination assay and chilling in a growth assay. However, of three lines tested in a single run of the drought assay, none performed better than wild-type.

Seven 35S::G3517 lines have been tested in pathogen plate assays to date, with variable results. Two lines showed decreased resistance to Botrytis infection, while two other lines were more resistant to Botrytis. One line showed enhanced resistance to Sclerotinia. 35S::G3517 plants showed developmental alterations when grown on plates, possibly influencing their performance in disease assays.

Potential applications. The results obtained to date indicate that constitutive expression of G3517 may confer enhanced tolerance to abiotic stress. G3517 overexpression increases Sclerotinia resistance.

G3518 (SEQ ID NO: 245 and 246; Glycine max)—Constitutive 35S

Background. G3518 is a closely-related soy homolog of G1792. The aim of this study was to determine whether overexpression of G3518 can confer abiotic stress tolerance and disease resistance similarly to G1792.

Morphological Observations. Forty two 35S::G3518 lines were isolated from 3 batches of T1 plants (Lines 301-309, 321-333 and 341-360). The overexpression of G3518 consistently induced dwarfing and dark green coloration in Arabidopsis. Overexpression lines were typically smaller than controls and considerable size variation was evident. Both late flowering (lines 344, 352 and 359) and early flowering (lines 303, 325, 326, 327 and 328) phenotypes were observed. It is interesting to note that all early flowering lines were observed in batch 1 and 2, and all late flowering lines were observed in batch 3. In many cases severe dwarfing was observed, and most lines exhibited narrow dark rather shiny contorted leaves. A small number of lines died prior to maturity.

Physiology (Plate assays) Results. Several 35S::G3518 lines performed better than wild-type seedlings in germination assays in the presence of NaCl and cold. These same lines also did well in a growth assay under cold conditions and in a C/N sensing assay. Several lines performed poorly in a heat growth assay: seedlings flowered earlier, suggesting they were stressed relative to wild-type and several had brown roots.

Physiology (Soil Drought-Clay Pot) Summary. Three lines performed better than controls in drought and/or drought recovery assays.

TABLE 87
35S::G3518 drought assay results:

| | | Mean | Mean | p-value for | Mean | | p-value for |
|---|---|---|---|---|---|---|---|
| | Project | drought | drought score | drought score | survival for | Mean survival | difference in |
| Line | Type | score line | control | difference | line | for control | survival |
| 323 | DPF | 2.0 | 1.4 | 0.053* | 0.37 | 0.33 | 0.45 |
| 323 | DPF | 1.3 | 0.50 | 0.0082* | 0.25 | 0.086 | 0.00042* |
| 326 | DPF | 1.7 | 1.6 | 0.53 | 0.34 | 0.34 | 0.90 |
| 326 | DPF | 0.70 | 0.50 | 0.40 | 0.11 | 0.050 | 0.082* |
| 333 | DPF | 2.1 | 2.1 | 0.87 | 0.39 | 0.42 | 0.63 |
| 333 | DPF | 1.3 | 0.60 | 0.043* | 0.23 | 0.12 | 0.020* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. The overexpression of G3518 consistently induced dwarfing and dark green coloration in Arabidopsis. A few severely affected lines died before maturity. Both early and late flowering lines were observed; these phenotypes may be environmentally influenced, since the occurrence of early vs. late flowering lines varied among different batches.

Several 35S::G3518 lines performed better than wild-type seedlings in germination assays in the presence of NaCl and cold. These same lines also did well in a growth assay under cold conditions, in a C/N sensing assay, and in a soil-based assay. In contrast, several lines performed poorly in a heat growth assay.

Potential applications. The results obtained to date indicate that constitutive expression of G3518 may be used to confer enhanced tolerance to abiotic stresses such as salt, cold, and nitrogen limitation. Given the developmental off-types associated with overexpression, this gene might need to be optimized by use of alternative promoters.

G3520 (SEQ ID NO: 241 and 242; Glycine max)—Constitutive 35S

Background. G3520 is a closely-related soy homolog of G1792. The aim of this study was to determine whether overexpression of G3520 confers abiotic stress tolerance and disease resistance similarly to G1792.

Morphological Observations. G3520 produced severe dwarfing when overexpressed. Almost all of the lines in each of three different batches were very small, slow developing, and exhibited curling/twisting dark, rather shiny leaves. The most severely affected individuals died at early stages. T1 plants were also typically late flowering, and generally exhibited a “strong G1792” phenotype. A total of twenty-eight new lines have been obtained: 321-328, 341-346 and 361-374.

Disease Summary. In experiments performed thus far, 3 of 8 lines tested were more sensitive to Botrytis than controls. Three separate lines were shown to be more resistant to Botrytis than controls, and 4 of 8 lines (none of which were Botrytis sensitive) were more resistant to Sclerotinia than control plants.

Physiology (Plate assays) Results. Four our of seven 35S::G3520 lines performed better than wild-type control seedlings in a C/N sensing assay. Two of these lines also did well in a growth assay under low nitrogen and chilling conditions.

Discussion. G3520 produced severe dwarfing when overexpressed. Almost all of the lines in each of three different batches were very small, slow developing, and exhibited twisted, dark rather shiny leaves. The most severely affected individuals died at early stages.

Four our of seven 35S::G3520 lines tested performed better than wild-type control seedlings in a C/N sensing assay. No soil drought or disease assays have been performed on these plants to date.

Potential applications. The results obtained to date indicate that constitutive expression of G3520 may confer enhanced tolerance to abiotic stresses such as nitrogen limitation and greater resistance to disease. However, given the developmental off-types associated with overexpression, the gene would likely need optimization. G3520 might also be applied to control developmental traits such as flowering time, coloration, and wax deposition.

G3737 (SEQ ID NO: 235 and 236; Oryza sativa)—Constitutive 35S

Background. G3737 is a closely-related rice homolog of G1792. The aim of this study was to determine whether overexpression of G3737 can confer abiotic stress tolerance and disease resistance similar to that induced by G1792.

Morphological Observations. G3737 produced severe dwarfing when overexpressed. Almost all of the lines in each of two different batches were very small, slow developing, and exhibited curling/twisting dark rather shiny leaves. Plants were typically small, spindly with slightly shiny leaves and were late flowering. At late stages of development, plants were bushy with stems bent at the nodes. Varying degrees of these phenotypes were evident and some lethality was also evident. A total of 35 new lines have been obtained: 301-315 and 321-340.

Physiology (Plate assays) Results. 35S::G3737 lines were tolerant to several abiotic stress assays. All ten lines were tolerant to cold in a germination assay. Five of these lines were tolerant to NaCl in another germination assay. Tolerance to severe dehydration was also seen in five lines. Three lines also were more tolerant to cold in a chilling growth assay.

Physiology (Soil Drought-Clay Pot) Summary. Three lines of 35S::G3737 overexpressors were more tolerant in soil-based drought assays than wild-type controls.

TABLE 88
35S::G3737 drought assay results:

| | | Mean | | p-value for | Mean | Mean | p-value for |
|---|---|---|---|---|---|---|---|
| | Project | drought | Mean drought | drought score | survival for | survival for | difference |
| Line | Type | score line | score control | difference | line | control | in survival |
| 304 | DPF | 2.5 | 1.7 | 0.011* | 0.52 | 0.36 | 0.0059* |
| 304 | DPF | 1.2 | 0.50 | 0.034* | 0.29 | 0.10 | 0.000097* |
| 308 | DPF | 2.8 | 1.6 | 0.00041* | 0.56 | 0.37 | 0.0020* |
| 308 | DPF | 1.7 | 0.90 | 0.041* | 0.31 | 0.16 | 0.0037* |
| 309 | DPF | 1.8 | 1.1 | 0.094* | 0.35 | 0.29 | 0.31 |
| 309 | DPF | 2.1 | 1.1 | 0.027* | 0.41 | 0.24 | 0.0016* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. G3737 produced severe dwarfing when overexpressed. Almost all of the lines were very small, slow developing, and exhibited curling/twisting dark, rather shiny leaves. These phenotypes were similar to those observed upon overexpression of other members of the G1792 clade, but on the severe side.

35S::G3737 lines showed enhanced tolerance in several abiotic stress assays. Ten out of ten lines were tolerant to cold in a germination assay. Five of these lines were tolerant to NaCl in another germination assay, and tolerance to severe dehydration was also seen in five lines. Three lines were significantly and consistently more tolerant to drought, and recovered from drought, better than controls.

Potential applications. The results obtained to date indicate that constitutive expression of G3737 may confer enhanced tolerance to abiotic stresses such as salt, cold, dehydration and drought. The gene might also be used to regulate developmental traits such as flowering time, coloration, and leaf shape. Given the off-types associated with overexpression, G3737 would likely need to be optimized by use of a tissue specific or inducible promoter.

### The G2053 Clade

G2053 (SEQ ID NO: 329 and 330; Arabidopsis thaliana)—Constitutive 35S

Background. G2053 was identified in the sequence of BAC T27C4, GenBank accession number AC022287, released by the Arabidopsis Genome Initiative.

Morphological Observations. A few 35S::G2053 were somewhat small in size, with a number of these overexpressors developing small rosettes and were early flowering. The remainder of the lines were similar to wild type in morphology and development.

Physiology (Soil Drought-Clay Pot) Summary. The function of G2053 was analyzed using transgenic plants in which the gene was expressed under the control of the 35S promoter. In a root growth assay on media containing high concentrations of PEG, G2053 overexpressors showed more root growth compared to wild-type controls. G2053 overexpressors also were significantly more drought tolerant than wild-type control plants. G2053 overexpressors flowered earlier than wild-type controls.

Drought assay results. One line of 35S::G2053 overexpressors was significantly more tolerant in soil-based drought assays than wild-type controls in three of four separate plantings.

TABLE 89
35S::G2053 drought assay results:

| | | Mean | | p-value for | Mean | Mean | p-value for |
|---|---|---|---|---|---|---|---|
| | Project | drought | Mean drought | drought score | survival for | survival for | difference in |
| Line | Type | score line | score control | difference | line | control | survival |
| 9 | DPF | 3.8 | 2.0 | 0.0023* | 0.44 | 0.27 | 0.15 |
| 9 | DPF | 1.3 | 1.0 | 0.41 | 0.10 | 0.33 | 0.15 |
| 9 | DPF | 3.9 | 1.2 | 0.00014* | 0.44 | 0.19 | 0.00000098* |
| 9 | DPF | 3.1 | 1.3 | 0.016* | 0.43 | 0.21 | 0.0000052* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

G516 (SEQ ID NO: 333 and 334; Arabidopsis thaliana)—Constitutive 35S

Morphological Observations. G516 overexpressors were similar to wild type in morphology and development.

Physiology Results. The function of G516 was analyzed using transgenic plants in which the gene was expressed under the control of the 35S promoter. G516 overexpressors were less sensitive to ABA than wild-type controls, and were significantly more cold tolerant than wild-type control plants. One line was shown to be more tolerant to drought than controls.

Potential Applications. G2053 or equivalogs could be used to increase yield, alter a plant's response water deficit conditions, and produce plants with enhanced tolerance to drought, salt stress, and freezing.

### The G2999 Clade

G2999 (SEQ ID NO: 255 and 256; Arabidopsis thaliana)—Constitutive 35S

Background. G2999 (AT2G18350) is an Arabidopsis protein from the ZF-HD family. 35S::G2999 lines examined during our earlier genomics screens showed a wild-type morphology and greater NaCl tolerance than controls. Some lines also showed enhanced seedling vigor in the absence of a stress treatment. 35S::G2999 lines performed well in a soil drought screen.

The aim of this study was to re-assess 35S::G2999 lines and compare its overexpression effects to those of the other ZF-HD proteins. We also sought to test whether use of a two-component overexpression system would produce any strengthening of the phenotype relative to use of a 35S direct promoter-fusion approach.

Morphological Observations. Additional set of 35S::G2999 lines have now been generated via both a direct promoter fusion and a two-component approach.

Line Details:

Lines 301-320 (Direct promoter-fusion): some of the lines were slightly small (#301, 302, 307, 309, 310, 314, 315) and the following lines flowered early: #301, 302, 307, 309-318. In other respects, this set of lines exhibited wild-type morphology.

Lines 681-700 (2-component): some of these lines were small (#684, 685, 694, 695, 699). In other respects, this set of plants showed wild-type morphology. Accelerated flowering was not observed.

Physiology (Plate assays) Results. Several of the 35S::G2999 lines (direct promoter fusion; P15277; T2 and T3 lines) lines had less root growth than controls on plates. However, some lines had root hairs that were much longer than control root hairs. Four T3 lines (generated during our earlier genomics program) were more tolerant to cold in a growth assay.

Subsequently, a set of 35S::G2999 two component lines were examined in a subset of the plate based assays. Four of these lines showed an enhanced performance in a sucrose germination assay. However, these lines were not tested in a cold growth assay.

Discussion. New sets of 35S::G2999 lines have been obtained using both a direct fusion approach and the two-component system. Both these sets of lines showed some size variation and a number of the lines were small at early stages. A significant number of lines from the direct promoter fusion set flowered early, but this phenotype was not seen in the two-component set, suggesting that it was dependent on there being a particular level of G2999 overexpression, or variables such as growth temperature, which might have differed between the plantings. Stress tolerance phenotypes were seen in plate based assays; the two-component lines showed improved tolerance in a sucrose germination assay, whereas the direct fusion lines showed improved tolerance in a cold growth assay (the two-component lines were not tested in the cold growth assay, but it is unclear at this stage why the direct fusion lines did not show a sucrose tolerance phenotype.) Root phenotypes were also apparent in the direct fusion lines: some lines had less root growth than wild-type, but other lines showed an increase in root hair length.

The two-component lines have recently been tested in soil drought assays and showed a markedly better performance than controls. Although these two-component lines have not been compared side-by-side with direct fusion lines in the same experiment, the drought phenotype was somewhat stronger in the former.

Potential applications. Based on the results obtained so far, G2999 could be applied to effect abiotic stress tolerance traits such as drought and cold tolerance. The gene might also be applied to modify traits such as flowering time and root development.

G2999 (SEQ ID NO: 255 and 256; Arabidopsis thaliana)—Leaf RBCS3

Background. The aim of this study was to assess whether expression of G2999 in photosynthetic tissue, from the RBCS3 promoter, is sufficient to confer stress tolerance.

Morphological Observations. Twenty-nine RBCS3::G2999 lines have been isolated (541-547, 941-942 and 981-100). Some size variation was apparent in the first two sets of lines, but otherwise, no consistent alterations in morphology were observed.

Physiology (Plate assays) Results. RBCS3::G2999 lines showed a better performance than controls in germination assays on mannitol (6 of 11 lines), NaCl (3 of 11 lines) and ABA plates (11 of 11 lines).

Physiology (Soil Drought-Clay Pot) Summary. Two RBCS3::G2999 lines were significantly more tolerant in soil-based drought assays than wild-type controls, on one of the planting dates for each overexpressor.

TABLE 90
RBCS3::G2999 drought assay results:

| | | Mean | | p-value for | Mean | Mean | p-value for |
|---|---|---|---|---|---|---|---|
| | Project | drought | Mean drought | drought score | survival for | survival for | difference in |
| Line | Type | score line | score control | difference | line | control | survival |
| 544 | TCST | 2.1 | 2.0 | 0.65 | 0.36 | 0.33 | 0.47 |
| 544 | TCST | 2.0 | 1.4 | 0.044* | 0.33 | 0.38 | 0.38 |
| 941 | TCST | 2.0 | 1.8 | 0.60 | 0.40 | 0.44 | 0.51 |
| 941 | TCST | 2.0 | 1.2 | 0.0075* | 0.39 | 0.27 | 0.032* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. We have isolated a set of RBCS3::G2999 lines via a two component approach. Some variation in size was seen among these plants, but overall no consistent alterations in morphology were observed. A number of the plate based assays have been completed, and an enhanced performance was seen relative to controls under osmotic stress (NaCl and mannitol) and on media containing ABA. Two lines showed evidence of greater drought tolerance than controls.

Potential applications. Based on the data so far, the RBCS3::G2999 combination may be used to confer tolerance to abiotic stresses such as drought and salinity.

G2999 (SEQ ID NO: 255 and 256; Arabidopsis thaliana)—Super Activation (N-GAL4-TA)

Background. The aim of this project is to determine whether the efficacy of the G2999 protein could be improved by addition of an artificial GAL4 activation domain.

Morphological Observations. A set of twenty 35S::GAL4-G2999 lines has been obtained. A number of these lines showed a slight acceleration in the onset of flowering, but otherwise the plants appeared wild type.

Physiological Results. 35S::GAL4-G2999 lines were more tolerant than wild-type controls to salt (3 of 10 lines tested), mannitol (3 of 10 lines), sucrose (5 of 10 lines), germination in heat (4 of 10 lines), severe desiccation (4 of 10 lines), and were less sensitive to ABA than control plants (10 of 10 lines).

Discussion. 35S::GAL4-G2999 have now been established; these lines showed a marginal acceleration in the onset of flowering.

Potential applications. The G2999 protein fused to such an activation domain may be used to modify traits such as flowering time. The stress tolerance effects of G2999 can be enhanced by addition of an artificial activation domain, particularly for heat and the hyperosmotic stresses salt, mannitol, sucrose and desiccation.

G2999 (SEQ ID NO: 255 and 256; Arabidopsis thaliana)—Point Mutation

Background. Overexpression constructs for a pair of different mutagenized forms of G2999 were made to examine the role of particular residues within the G2999 protein, and for purposes of functional optimization.

The following variants have been made:
(1) P25737: 35S::G2999(W249G) (targets conserved W residue in homeodomain).
(2) P25736: 35S::G2999(CHR123-125GLL) (targets conserved CHR residues of the zinc finger domain).

Morphological Observations. A set of overexpression lines for each of two G2999 variants has now been obtained. In each case, the majority of lines showed a wild-type phenotype.

Variant (1) Lines 1041-1060 (containing P25737, 35S::G2999(W249G)): 1043, 1044, 1048, 1052, 1058 were small at early stages. #1051, 1053, 1054, 1057, 1060 were slightly early flowering. Otherwise, no consistent effects on morphology were observed.

Variant (2) Lines 1021-1040 (containing P25736, 35S::G2999(CHR123-125GLL): all appeared wild type except for #1024-1026, 1032, 1038 which were slightly early flowering.

Physiology (Plate assays) Results. In tests performed thus far, several of the lines harboring mutagenized variants of G2999 were more tolerant, relative to wild-type controls, in plate-based abiotic stress assays. Seven of 10 lines overexpressing site-directed mutation (2) had a higher rate of germination than controls on mannitol, 4/10 on sucrose, 10 of 10 were less sensitive to ABA, and 3 of 10 were more tolerant to desiccation.

Three of 10 lines overexpressing site-directed mutation (1) had a higher rate of germination than controls on sucrose; the bulk of the remaining assays have not been completed at this time.

Discussion. We have now obtained a set of overexpression lines for each of the above constructs. In each case, the plants showed no consistent changes in morphology.

Potential Applications. Lines overexpressing G2999 with particular site-directed mutations are likely to have better quality and greater yield in several types of abiotic stress conditions, including hyperosmotic stresses.

G2991 (SEQ ID NO: 281 and 282; Arabidopsis thaliana)—Constitutive 35S

Background. G2991 is an Arabidopsis protein from the ZF-HD family and is a closely-related homolog to G2999. 35S::G2991 lines were examined during our earlier genomics screens: these lines showed reduced size in some cases, but otherwise has wild-type morphology. A wild-type phenotype was obtained in stress assays.

Morphological Observations. Three new sets of 35S::G2991 lines have been obtained: 301-302, 321-323, 341-360. Many of these lines were reduced in size to various extents. Four out of ten 35S:G2991 lines had more root growth and root hairs on control plates compared to wild-type seedlings.

Discussion. New sets of 35S::G2991 lines have been obtained; as in the genomics screens, some size variation was apparent but there were no consistent alterations in morphology. In plate based assays, no effects on stress tolerance were seen, but many of the lines showed enhanced root growth compared to controls and exhibited an increase in root hair density.

Potential applications. Based on the results obtained so far, G2991 could be applied to enhance root development, which might improve traits such as drought tolerance and nitrogen use efficiency.

G2989 (SEQ ID NO: 279 and 280; Arabidopsis thaliana)—Constitutive 35S

Background. G2989 is an Arabidopsis protein from the ZF-HD family and is a closely-related homolog to G2999. 35S::G2989 lines were examined during our earlier genomics screens: some of the lines showed an early flowering phenotype, but otherwise, a wild-type response was observed in all assays performed.

Morphological Observations. Two new sets of 35S::G2989 direct promoter fusion lines have been generated: #301-320 and #321-340. Some size variation was noted in these sets of lines at early stages, but overall, no consistent differences to wild-type were apparent. However, 2/20 lines from the second set of plants (T1-327 and T1-335) showed accelerated flowering.

Physiology (Plate assays) Results. Six out of ten 35S::G2989 lines were more tolerant to a severe dehydration stress compared with wild-type control seedlings. Five of ten lines were also more tolerant to cold conditions in a growth assay.

Physiology (Soil Drought-Clay Pot) Summary. Three lines of 35S::G2989 overexpressors performed better than controls in drought and/or drought recovery assays.

TABLE 91
35S::G2989 drought assay results:

| | | Mean | Mean | p-value for | Mean | | p-value for |
|---|---|---|---|---|---|---|---|
| | Project | drought | drought score | drought score | survival for | Mean survival | difference in |
| Line | Type | score line | control | difference | line | for control | survival |
| 302 | DPF | 0.67 | 1.0 | 1.0 | 0.12 | 0.17 | 0.30 |
| 302 | DPF | 1.3 | 1.2 | 0.79 | 0.26 | 0.25 | 0.78 |
| 302 | DPF | 1.8 | 1.2 | 0.028* | 0.29 | 0.25 | 0.27 |
| 311 | DPF | 1.7 | 1.0 | 0.41 | 0.29 | 0.17 | 0.055* |
| 318 | DPF | 2.2 | 1.5 | 0.0082* | 0.40 | 0.30 | 0.032* |
| 318 | DPF | 1.6 | 0.70 | 0.048* | 0.31 | 0.17 | 0.0059* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Two new sets of 35S::G2989 lines have been obtained. These lines showed a wild-type morphology except for a small number of individuals that flowered early. However, stress tolerance was shown by a substantial number of lines in both plate-based severe dehydration and cold growth assays. One of three lines tested also showed a better performance than wild-type in a single run of the soil drought clay pot assay. It is noteworthy that the highly related gene, G2990 also gave cold and drought tolerance when overexpressed, indicating that these two proteins have equivalent activities.

Potential applications. Based on the results obtained so far, G2989 could be applied to effect abiotic stress tolerance traits such as drought and cold tolerance.

G2990 (SEQ ID NO: 283 and 284; Arabidopsis thaliana)—Constitutive 35S

Background. G2990 is an Arabidopsis protein from the ZF-HD family and is a closely-related homolog to G2999. 35S::G2990 lines were examined during our earlier genomics screens: these lines showed wild-type morphology, showed increased sensitivity to low N in plate based assays.

Morphological Observations. Most of a set of twenty 35S::G2990 T1 lines (#301-320) showed no consistent alterations in morphology. Some size variation was apparent and a number of lines (310, 312, 314) were slightly dark in coloration.

Physiology (Plate assays) Results. Five out of ten lines were insensitive to ABA in a germination assay. Three out of ten lines were more tolerant to cold conditions in a growth assay. A number of lines showed less extensive root development than controls when grown on plates without a stress treatment.

Physiology (Soil Drought-Clay Pot) Summary. Three independent lines have been tested in a single ran of the soil drought assay. Two of these lines performed significantly better than wild-type controls.

TABLE 92
35S::G2990 drought assay results:

| | | Mean | Mean | p-value for | Mean | Mean | p-value for |
|---|---|---|---|---|---|---|---|
| | Project | drought | drought | drought score | survival for | survival for | difference in |
| Line | Type | score line | score control | difference | line | control | survival |
| 312 | DPF | 2.8 | 0.44 | 0.0021* | 0.42 | 0.095 | 0.00000071* |
| 313 | DPF | 1.8 | 0.44 | 0.038* | 0.40 | 0.095 | 0.0000040* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. A new set of 35S::G2990 lines has been obtained. These lines showed no consistent alterations in morphology. However, stress tolerance was shown by a substantial number of lines in both plate-based ABA and cold growth assays. However, some of the lines showed reduced root development compared to wild-type when grown on agar plates in the absence of stress treatments. Two of three lines tested also showed a better performance than wild-type in a single run of the soil drought clay pot assay. It is noteworthy that the highly related gene, G2989 also gave cold and drought tolerance when overexpressed, indicating that these two proteins have equivalent activities.

Potential applications. Based on the results obtained so far, G2990 could be applied to effect abiotic stress tolerance traits such as drought and cold tolerance.

G3000 (SEQ ID NO: 259 and 260; Arabidopsis thaliana)—Constitutive 35S

Background. G3000 is an Arabidopsis protein from the ZF-HD family and is a closely-related homolog to G2999.

Morphological Observations. Twenty 35S::G3000 T1 lines have been selected: at early stages, considerable size variation was apparent among these lines and a significant number (#302, 305, 311, 312, 314, 318) were distinctly early flowering. Some of the lines also showed floral abnormalities.

Discussion. A new sets of 35S::G3000 lines have been obtained; many of the lines were early flowering and were reduced in size at early stages. These lines have been tested in plate based assays and showed a wild-type response.

Potential applications. Based on the results obtained so far, G3000 could be applied to manipulate developmental traits such as flowering time.

G3676 (SEQ ID NO: 265 and 266; Zea mays)—Constitutive 35S

Background. G3676 is a ZF-HD family gene from maize and is a closely-related homolog to G2999. We are testing whether example ZF-HD genes from a number of different species, can confer drought tolerance in a comparable manner to G2999.

Morphological Observations. Overexpression of G3676 in Arabidopsis produced alterations in flowering time and a reduction in plant size.

Line Details: T1 Lines 301-320: #305, 306, 308, 312, 313, 317, 319, 320 were small. #307, 311, 318 died at early stages. Lines 301-304, 308-310, 312, 314-316, 320 flowered early. Lines 305, 306, 313, 319 were slow developing and flowered slightly late. The remaining lines appeared wild type.

Physiology (Plate assays) Results. Six of ten 35S::G3676 lines had very good tolerance towards NaCl in a germination assay. Four lines also had very good tolerance towards severe dehydration in a plate assay.

Discussion. Overexpression of G3676 produced a reduction in overall size and accelerated flowering in many of the lines. 35S::G3676 lines showed an enhanced performance versus wild type in NaCl germination assays and in a severe dehydration assay.

Potential applications. Based on the results so far, G3676 may be used to confer tolerance to abiotic stresses such as salinity and drought. The gene might also be used to regulate developmental traits such as flowering time.

G3681 (SEQ ID NO: 277 and 278; Zea mays)—Constitutive 35S

Background. G3681 is a ZF-HD family gene from maize and is a closely-related homolog to G2999. We are testing whether example ZF-HD genes from a number of different species, can confer drought tolerance in a comparable manner to G2999.

Morphological Observations. Overexpression of G3681 in Arabidopsis produced alterations in flowering time and a reduction in plant size. Some of the overexpression lines also exhibited a wrinkled silique phenotype.

Line Details:

T1 Lines 301-320: #302, 304, 306, 307, 308, 309, 312, 313, 314, 316 were small. #305, 315, 317 perished at early stages. #301, 306, 310, 311, 318, 319 flowered early. #301, 306, 318, 319 had wrinkled siliques.

T1 lines 321-340: 325, 326, 327, 328, 329, 331, 333-336, 340 were small, late developing, and dark in coloration. The following lines flowered early: 321, 322, 324, 331, 332, 334, 337-339. All except 324, 327, 332, 339 had slightly wrinkled siliques.

Physiology (Plate assays) Results. Five out of ten 35S::G3681 lines were more tolerant to NaCl in a germination assay.

Discussion. Overexpression of G3681 produced a reduction in overall size and accelerated flowering in many of the lines. Many of the lines also showed abnormal silique development and had somewhat wrinkled siliques. 35S::G3681 lines showed an enhanced performance versus wild type in an NaCl germination assay on plates.

Potential applications. G3681 may be used to confer tolerance to abiotic stresses such as salinity. The gene might also be used to regulate developmental traits such as flowering time and fruit development.

G3686 (SEQ ID NO: 267 and 268; Oryza sativa)—Constitutive 35S

Background. G3686 is a ZF-HD family gene from rice and is a closely-related homolog to G2999. We are testing whether example ZF-HD genes from a number of different species, can confer drought tolerance in a comparable manner to G2999.

Morphological Observations. All of the 35S::G3686 T1 lines (301-320) showed a mild early flowering phenotype. A number of the lines were also markedly small (#301, 303, 305, 306, 308, 310, 312, 314, 315, 316, 318, 319), but in other respects, this set of plants exhibited wild-type morphology.

Physiology (Plate assays) Results. Three out of ten 35S::G3686 lines were more tolerant to cold conditions in a germination assay. Seedlings contained less anthocyanins and were larger than wild-type seedlings.

Physiology (Soil Drought-Clay Pot) Summary. Three independent lines have been tested in soil drought assays and each line performed significantly better than wild-type controls.

TABLE 93
35S::G3686 drought assay results:

| | | Mean | Mean | p-value for | Mean | Mean | p-value for |
|---|---|---|---|---|---|---|---|
| | Project | drought | drought score | drought score | survival for | survival for | difference in |
| Line | Type | score line | control | difference | line | control | survival |
| 303 | DPF | 2.2 | 1.1 | 0.0099* | 0.44 | 0.30 | 0.014* |
| 303 | DPF | 2.0 | 1.3 | 0.022* | 0.41 | 0.39 | 0.63 |
| 306 | DPF | 2.3 | 1.6 | 0.011* | 0.56 | 0.42 | 0.024* |
| 306 | DPF | 2.2 | 1.2 | 0.031* | 0.42 | 0.33 | 0.11 |
| 309 | DPF | 2.1 | 1.3 | 0.023* | 0.51 | 0.31 | 0.00060* |
| 309 | DPF | 1.7 | 0.90 | 0.10* | 0.25 | 0.14 | 0.017* |

DPF = direct promoter fusion project
Surival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Overexpression of G3686 produced deleterious effects in Arabidopsis; 35S::G3686 lines were slightly early flowering and many of the lines were smaller than controls. In plate based germination assays, enhanced tolerance to cold was observed in these lines. Three lines were significantly and consistently more tolerant to drought than controls.

Potential applications. Based on the data obtained so far, G3686 may be used to modify developmental traits such as flowering time and abiotic stress related traits such as cold and drought tolerance.

### The G3086 Clade

G3086 (SEQ ID NO: 291 and 292; Arabidopsis thaliana)—Constitutive 35S

Background. G3086 was included in the drought program because it showed good tolerance towards heat and salt stress in the abiotic stress assays. The gene is also abiotic stress responsive based on transcript profiling experiments indicating that it could have an endogenous function in such stress responses. The aim of this study was to re-assess 35S::G3086 lines and compare the overexpression effects with those of its closely-related homologs.

Morphological Observations. Twelve new 35S::G3086 direct fusion lines (301-320) and twenty-five new 35S::G3086 two-component lines (321-326 and 381-399) were isolated. The majority of lines were dwarfed and showed accelerated flowering.

Direct fusion lines: Line 306 died early in development. All other lines were small, spindly and early flowering.

Two-component lines (321-326): Line 321 was small, spindly and early flowering—similar to the direct fusion lines. Line 325 died early in development. Lines 322, 323 and 324 were small, dark green and late flowering. Line 326 was largely wild type in appearance.

Two-component lines (381-399): All lines (except line 384) were small, compared to controls, early in development. Lines 382, 384, 390, 392, 393 and 394 were small, spindly and early developing vs. control. All other lines were not significantly different from control plants.

Physiology (Plate assays) Results. Two generations (T2 and T3) of 35S::G3086 direct fusion lines have been tested in abiotic stress assays.

Severe dehydration tolerance was observed in 4 of 10 direct promoter fusion lines. Three out of 10 direct promoter fusion lines tested also showed a better performance than controls under cold growth conditions. Using a two-component expression system, 3 of 12 overexpressing lines were more tolerant to mannitol.

Discussion. New sets of 35S::G3086 plants have been obtained by both a direct promoter fusion and a two-component approach. Both sets of lines displayed early flowering and significant dwarfing. In plate based assays, a few lines were tolerant to severe dehydration and cold stress during seedling growth. While a few lines also were tolerant to heat and salt stress in plate based assays, the magnitude of the stress tolerance was not as great as that seen during the original genomics program. Because of the dwarfing observed, the lines submitted for abiotic stress assays may have had a weaker phenotype than those originally isolated during the genomics program. Some evidence of drought tolerance has been obtained in soil assays, but the data presented here were obtained with the original lines from the genomics program.

Potential applications. At this stage of the analysis, it appears that G3086 may be useful for creating tolerance to abiotic stress such as hyperosmotic stress (e.g., drought) and cold in crops. It is possible that constitutive expression of G3086 might also be useful for modifying developmental traits such as flowering time and plant size.

G3086 (SEQ ID NO: 291 and 292; Arabidopsis thaliana)—Knockout (KO)

Background. It would be useful to characterize a knockout mutant of G3086 in order to assign a role to the endogenous G3086 locus, and would help determine whether the gene has a native function in growth or regulation of stress responses.

Morphological Observations. A G3086 T-DNA insertion line derived from the SALK collection, SALK_{—}049022, was obtained from the ABRC at Ohio State University (NCBI acc. no. CC05386, version CC053826.1; GI:29473490; SALK_{—}049022.30.00.x Arabidopsis thaliana TDNA insertion lines Arabidopsis thaliana genomic clone SALK_{—}049022.30.00.x, genomic survey sequence). BLAST analysis of the sequence from the insertion point deposited in GenBank by SALK indicates that the T-DNA in this line is integrated approximately 143 bp downstream of the G3086 start codon.

We identified 1 homozygous plant (lines 10), by PCR genotyping, among eleven individuals germinated from the seed lot supplied by the ABRC. Selfed seed was collected from the homozygous plant and a batch of 6 progeny were grown from line 10 and examined under 24-hour light conditions. These plants showed a moderate delay in flowering compared to wild-type controls.

Discussion. We have isolated a homozygous population for a T-DNA insertion allele of G3086 from the SALK collection. Interestingly, these plants showed delayed flowering, a converse phenotype to that seen in the overexpression lines. Such effects indicate that G3086 could have an endogenous role in regulation of the floral transition.

Potential applications. The morphological data obtained from the KO.G3086 line support our conclusions that G3086 can be a useful transcription factor for manipulating flowering time. In particular, a knock-down approach on this gene or its homologs might have utility in delaying the onset of flowering.

G3086 (SEQ ID NO: 291 and 292; Arabidopsis thaliana)—Root-Specific RSI1

Background. G3086 was included in the drought program because it showed good tolerance towards heat and salt stress in the abiotic stress assays performed during the genomics program. The gene is also abiotic stress responsive (based on transcript profiling experiments) indicating that it could have an endogenous function in such stress responses. The aim of this study was to determine whether the morphological (such as early flowering and dwarfing) and stress tolerance effects of G3086 overexpression could be separated through root-specific expression with the RSI1 promoter.

Morphological Observations. Twenty RSI1::G3086 lines have been planted (301-320). Nine lines showed no differences to control plants. However, a significant number of lines ( 8/20) were early flowering and had slightly flat leaves (805, 807, 809, 811, 812, 815, 817 and 818). A single line, #816 had a rather dark shiny appearance. Severe dwarfing was not seen in any of the lines, and at later stages they appeared wild type.

Discussion. A significant number of RSI1::G3068 plants were early flowering compared to wild-type control seedlings in morphological assays. However, the lines were of wild-type size. Thus, the severe dwarfing observed in 35S::G3086 lines have been eliminated using root-specific expression. The result is interesting, though, as it indicates that G3086 activity in the root might constitute a developmental signal that triggers the onset of flowering.

Potential applications. The RSI1::G3086 combination may be useful for manipulating flowering time without conferring deleterious morphological or developmental effects.

G3086 (SEQ ID NO: 291 and 292; Arabidopsis thaliana)—Emergent Leaf Primordia-Specific AS1

Background. The aim of this study was to determine whether the morphological (such as early flowering and dwarfing) and stress tolerance effects of G3086 overexpression could be separated through Emergent leaf primordia-specific expression with the AS1 promoter.

Morphological Observations and Discussion. AS1::G3086 plants were slightly small and some T1 lines flowered marginally earlier than wild-type control plants in morphological assays. These phenotypes were not as penetrant as in 35S::G3086 lines, and it therefore appears that expression from the AS1 promoter does not produce many of the deleterious effects of G3086 expression.

Potential applications. The AS1::G3086 combination may be useful for manipulating flowering time without conferring deleterious morphological or developmental effects.

G3086 (SEQ ID NO: 291 and 292; Arabidopsis thaliana)—Double Overexpression

Background. The aim of this double overexpression project is to determine whether different transcription factor genes will give an additive effect on drought/disease/low N tolerance when “stacked” together in the same line.

Morphological Observations.

35S::G481 line 3 (female)×35S::G3086 line 8 (male). Twenty F1 plants were obtained. All showed an identical phenotype to the 35S::G3086 parental line: very early flowering, reduced size, and spindly inflorescences.

35S::G1073 line 4 (male)×35S::G3086 line 8 (female). Eleven F1 plants were obtained. All F1 plants showed an intermediate leaf phenotype, but were earlier flowering than wild-type. Thus, the accelerated flowering produced by G3086 overexpression appears epistatic to the delayed flowering associated with G1073 overexpression. The double overexpressing plants had broader leaves than the 35S::G3086 parental line, but the leaves were smaller than in wild-type and the 35S::G1073 parental line.

Discussion. A 35S::G481;35S::G3086 line has been made by crossing. F1 plants have recently been obtained, and these all showed a 35S::G3086-like morphology; the plants were very early flowering.

A cross has also been made to create a 35S::G3086;35S::G1073 line. The aim of this combination was to determine whether the accelerated flowering seen in 35S::G3086 lines would compensate for the delayed flowering seen in some of the 35S::G1073 lines. We have now obtained F1 plants from the cross; these plants flowered earlier than wild-type, demonstrating that overexpression of G3086 can overcome the delay in flowering that results from G1073 overexpression. The leaves of the double overexpression line were broader and rounder than those of the 35S::G3086 parental line, but were smaller than those of wild-type and the 35S::G1073 parental line.

Potential applications. The 35S::G3086;35S::G1073 and 35S::G3086;35S::G481 combinations indicate that a stacking approach might offer advantages over use of the 35S::G1073 or the 35S::G481 transgenes alone. For example, both 35S::G1073 and 35S::G481 in soybean produce a delayed flowering off-type which is associated with a yield penalty (field trial results). Combining G1073 or G481 with G3086 overexpression in the same soy line may afford drought tolerance without the delayed flowering caused by G1073 or G481.

G2555 (SEQ ID NO: 317 and 318; Arabidopsis thaliana)—Constitutive 35S

Background. G2555 was included in the drought program because it is an Arabidopsis sequence related to G3086. Previously, during our earlier genomics screen we found that 35S::G2555 lines exhibited accelerated flowering, constitutive photomorphogenesis and increased sensitivity to Botrytis. The aim of this study was to re-assess 35S::G2555 lines and compare the overexpression effects with those of its homologs.

Morphological Observations. Thirty-two 35S::G2555 lines have been isolated (301-312 and 341-360).

Lines 301-312: Line 302 died early in development. All other lines were slightly small, spindly and early flowering (except 301, 305 and 312). Line 312 was dark green with high anthocyanin in the vasculature and secondary shoots.

Lines 341-360: Initially lines 346, 347, 349, 356, 357 and 360 were early developing with small rosettes. Later, all lines were somewhat small and spindly.

Physiology (Plate assays) Results. Nine out of ten 35S::G2555 lines were more tolerant to heat in a growth assay. Three of these lines were also more tolerant to cold in another growth assay. In the latter assay, the seedlings were slightly larger and contained less anthocyanin.

Discussion. 35S::G2555 lines were slightly small and showed a marked acceleration of flowering, confirming the results of our genomics screens. Heat tolerance was observed in nine 35S::G2555 lines and cold tolerance was also observed in three lines.

Importantly, we found that 35S::G2555 tomato lines grown during the trials funded by an ATP grant (performed in the summer of 2004) showed more rapid growth and increased biomass and vigor compared to controls under hot, dry field conditions.

Potential applications. The results of these overexpression studies confirm that G2555 could be a good candidate gene for improvement of abiotic stress tolerance (such as drought, heat, and cold) in commercial species. The gene might also be applied to improve vigor and manipulate the onset of flowering. It should be pointed out that the disease susceptibility that we detected in 35S::G2555 lines is of potential concern. Further experimentation will be needed to determine how the increased drought tolerance is related to this off-type.

G3750 (SEQ ID NO: 325 and 326; Oryza sativa)—Constitutive 35S

Background. G3750 was included in the drought program because it is a closely-related rice homolog of G3086. The aim of this study was to assess 35S::G3750 lines and compare the overexpression effects with those of the other genes from the G3086 study.

Morphological Observations. Twenty 35S::G3750 lines have been isolated (301-320). A large amount of size variation was seen in this set of T1 plants and many of the lines were noted to be slightly early flowering: #301, 304, 305, 309, 314, 315, 316, 317, 318, 319. Many of the lines were small.

Physiology (Plate assays) Results. 35S::G3750 lines showed a significantly better performance than wild type in an ABA germination assay (7 of 10 lines), the severe dehydration assay (9 of 10 lines), and the heat growth assay (6 of 10 lines).

Discussion. 35S::G3750 plants were small compared to wild-type control seedlings and a number of lines were noted to be slightly early flowering. 35S::G3750 lines showed a good performance in plate based ABA germination, severe dehydration and heat growth assays. These phenotypes are comparable to those produced by G3086 overexpression, indicating that the G3750 and G3086 proteins have similar activities.

Potential applications. Based on available results, G3750 is an excellent candidate for producing tolerance to abiotic stress in crops. The gene could also be used to modify traits such as flowering time.

G3760 (SEQ ID NO: 323 and 324; Zea mays)—Constitutive 35S

Background. G3760 was included in the drought program because it is a closely-related maize homolog of G3086. The aim of this study was to assess 35S::G3760 lines and compare the overexpression effects with those of its homologs.

Morphological Observations. Twenty 35S::G3760 lines have been isolated (301-320); the majority of these showed a marked acceleration in the onset of flowering and were distinctly small and rather pale in coloration.

Details:

Lines 303, 309, 317 and 320 died early in development. Lines 308, 312 and 313 had abnormal phyllotaxy and were small (50%) in size. All others were pale and generally very small. Lines 304, 306, 307, 310 and 315 had slightly wrinkled, short, broad siliques vs. control plants. In general, the lines flowered early, although the degree of early flowering varied.

Physiology (Plate assays) Results. Four of 10 35S::G3760 lines were more tolerant to salt than wild-type controls. Five of 10 overexpressing lines were more tolerant to germination in cold conditions than wild type. Five of 10 lines were also less sensitive or insensitive to ABA (as compared to ABA-sensitive wild-type controls).

Discussion. 35S::G3760 lines were markedly early flowering, pale in coloration, small and showed growth abnormalities such as deformed siliques. Positive results were observed in plate assays: 35S::G3760 lines were tolerant to NaCl, ABA and cold in germination assays. Similar phenotypes are seen in 35S::G3086 lines indicating that G3086 and G3760 have comparable activities.

Potential applications. The results of these overexpression studies confirm that G3760 could be a good candidate gene for improvement of drought related stress tolerance in commercial species. However, the decrease in size, paleness, and early flowering seen in some of the lines suggests that the gene might require optimization by use of different promoters or protein modifications, prior to product development. The gene might also be useful for regulation of developmental traits like flowering time.

G3765 (SEQ ID NO: 313 and 314; Glycine max)—Constitutive 35S

Background. G3765 was included in the drought program because is a closely-related soybean homolog of G3086. The aim of this project was to assess 35S::G3765 lines and compare the overexpression effects with those of other genes from the G3086 study.

Morphological Observations. Twenty 35S::G3765 lines were isolated (301-320). Considerable size variation was apparent and many of the lines were somewhat small, particularly at early stages. Lines 301, 302, 303 and 315 appeared wild type. Three lines (301, 314 and 315) were early developing compared to control plants

Physiology (Plate assays) Results. Four out of ten 35S::G3765 lines were more insensitive to ABA than control seedlings.

Physiology (Soil Drought-Clay Pot) Summary. Two lines performed significantly better than CBF4-overexpressing (OEX) control plants (these controls are more drought tolerant than wild-type).

TABLE 94
35S::G3765 drought assay results:

| | | | | Mean | | | | |
|---|---|---|---|---|---|---|---|---|
| | | | Mean | drought | p-value for | Mean | Mean | p-value for |
| | Project | | drought | score | drought score | survival | survival for | difference in |
| Line | Type | Control | score line | control | difference | for line | control | surival |
| 302 | DPF | G912 OEX | 2.3 | 1.2 | 0.066* | 0.47 | 0.24 | 0.000083* |
| 302 | DPF | G912 OEX | 0.70 | 0.50 | 0.55 | 0.17 | 0.086 | 0.035* |
| 305 | DPF | G912 OEX | 3.7 | 1.7 | 0.0073* | 0.75 | 0.26 | 0.0000000016* |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion: Overall, 35S::G3765 lines were not consistently different from wild-type plants in morphological assays, except for some variation in overall size. A slight acceleration in flowering time was seen in a small number of lines, but this phenotype needs to be confirmed. Insensitivity to ABA was also noted in four lines during plate based assays. Two lines showed significant drought tolerance relative to controls.

Potential applications: Based on the results from plate assays, G3765 might be used to confer abiotic stress tolerance, including drought tolerance.

G3766 (SEQ ID NO: 303 and 304; Glycine max)—Constitutive 35S

Background. G3766 was included in the drought program because is a closely-related soybean homolog of G3086. The aim of this project was to assess 35S::G3766 lines and compare the overexpression effects with those of other genes from the G3086 study.

Morphological Observations. Twenty 35S::G3766 lines were isolated (301-320); a significant number of lines showed accelerated flowering (301, 302, 305, 307, 308, 309, 310, 312, 314, 315 and 317) but otherwise, apart from some size variation, the lines generally appeared wild type.

Physiology (Plate assays) Results. Seven of 10 35S::G3760 lines were more tolerant to severe dehydration assays than wild-type controls. Three of 10 overexpressing lines were more tolerant to germination in cold conditions than wild type. Seven of 10 lines were also less sensitive or insensitive to ABA (as compared to ABA-sensitive wild-type controls).

Discussion. Overexpression of G3766 accelerated flowering in Arabidopsis. In other respects, though, the lines appeared wild type. Given that G3086 also accelerates the onset of flowering when overexpressed, the two proteins may have similar activities.

Potential applications. G3766 can be used to manipulate developmental traits such as flowering time, and confer cold and hyperosmotic stress (e.g., salt) tolerance to plants.

G3767 (SEQ ID NO: 297 and 298; Glycine max)—Constitutive 35S

Background. G3767 was included in the drought program because it is a closely-related soybean homolog of G3086. The aim of this study was to assess 35S::G3767 lines and compare the overexpression effects with those of other G3086 related genes.

Morphological Observations. Twenty-seven 35S::G3767 lines were isolated (301-317 and 321-330). G3767 overexpression induced slightly early flowering in most lines. Most lines were also somewhat smaller than controls, although line 330 was wild-type sized, and 317 was larger than controls. Lines 315, 329 and 330 were markedly early flowering. A small number of lines (301, 324 and 325) were slightly late developing.

Physiology (Plate assays) Results. Three out of ten 35S::G3767 lines were more tolerant than wild-type seedlings in a severe dehydration based assay. Three of ten G3767 overexpressing lines were less sensitive to ABA than wild type. Five 35S::G3767 lines had more root growth compared to wild-type seedlings in a root growth assay.

Discussion. The majority of 35S::G3767 plants were slightly early flowering, but otherwise appeared wild type. A number of 35S::G3767 lines were more stress tolerant than controls based on results from a severe plate based dehydration assay. Some lines also had more root growth in plate grown conditions.

Potential applications. The results of these overexpression studies confirm that G3767 could be a candidate gene for improvement of drought related stress tolerance in commercial species. Additionally, the gene may be used to manipulate developmental traits such as flowering time and root structure.

G3768 (SEQ ID NO: 293 and 294; Glycine max)—Constitutive 35S

Background. G3768 was included in the drought program because it is a closely-related soybean homolog of G3086. The aim of this study was to assess 35S::G3768 lines and compare the overexpression effects with those of other G3086 related genes.

Morphological Observations. Thirty 35S::G3768 lines have been isolated (301-314 and 321-336).

Lines 301-314: All lines were early flowering compared to controls. Line 306 was larger than controls and line 311 was small. No other differences between G3768 overexpression lines and controls were evident.

Lines 321-336: The sizes and flowering times in this batch of plants were more variable, including the controls. Overall, however, the trend was again towards early flowering and no other consistent differences.

Physiology (Plate assays) Results. Five of ten G3768 overexpressing lines were less sensitive to ABA than wild-type controls.

Discussion. 35S::G3768 lines were early flowering, a comparable effect to that seen on G3086 overexpression. However, when these lines were tested in plate based assays, a wild-type response was observed under all conditions.

Potential applications. Based on the data so far, G3768 may be used to regulate the floral transition and to confer abiotic stress tolerance to plants, including commercial species.

G3769 (SEQ ID NO: 295 and 296; Glycine max)—Constitutive 35S

Background. G3769 is a closely-related soybean homolog of G3086. The aim of this study was to assess 35S::G3769 lines and compare the overexpression effects with those of other G3086 related genes.

Morphological Observations. Eighteen 35S::G3769 lines were isolated (301-318). Fourteen lines were early flowering. In other respects, these lines showed wild-type morphology.

Physiology (Plate assays) Results. Three of ten 35S::G3769 overexpressing lines were less sensitive to ABA than wild-type controls, and 6 of 10 overexpressing lines were more tolerant to severe desiccation than wild-type controls.

Discussion. 35S::G3769 lines were early flowering, a comparable effect to that seen on G3086 overexpression.

Potential applications. Based on the data so far, G3769 may be used to regulate the floral transition and to confer drought and/or other abiotic stress tolerance to plants, including commercial species.

G3771 (SEQ ID NO: 311 and 312; Glycine max)—Constitutive 35S

Background. G3771 is a closely-related soybean homolog of G3086. The aim of this study was to assess 35S::G3771 lines and compare the overexpression effects with those of its homologs.

Morphological Observations. Twenty-four 35S::G3771 lines were isolated (321-324 and 341-360). Over three-quarters of the lines were early-flowering, slightly pale, and all lines were dwarfed to varying extents. Some of the lines had floral defects and produced deformed siliques (#344, 347, 349, 350 and 359).

Physiology (Plate assays) Results. Seven of 10 overexpressing lines were more tolerant to severe desiccation in plate-based assays than wild-type controls.

Physiology (Soil Drought-Clay Pot) Summary. Three lines performed significantly better than CBF4-overexpressing (OEX) control plants (these controls are more drought tolerant than wild-type).

TABLE 95
35S::G3771 drought assay results:

| | | | Mean | Mean | p-value for | Mean | Mean | p-value for |
|---|---|---|---|---|---|---|---|---|
| | Project | | drought | drought | drought score | survival | survival for | difference in |
| Line | Type | Control | score line | score | difference | for line | control | survival |
| 323 | DPF | CBF4 | 1.8 | 1.6 | 0.60 | 0.41 | 0.31 | 0.083* |
| | | OEX | | | | | | |
| 323 | DPF | CBF4 | 0.70 | 0.70 | 0.83n | 0.11 | 0.14 | 0.59 |
| | | OEX | | | | | | |
| 344 | DPF | CBF4 | 1.3 | 0.80 | 0.13 | 0.17 | 0.14 | 0.51 |
| | | OEX | | | | | | |
| 344 | | CBF4 | 2.2 | 0.90 | 0.0040* | 0.47 | 0.29 | 0.0028* |
| | | OEX | | | | | | |
| 347 | | CBF4 | 1.3 | 0.90 | 0.20 | 0.16 | 0.11 | 0.23 |
| | | OEX | | | | | | |
| 347 | | CBF4 | 1.9 | 0.90 | 0.033* | 0.39 | 0.18 | 0.00014* |
| | | OEX | | | | | | |

DPF = direct promoter fusion project
Survival = proportion of plants in each pot that survived
Drought scale: 6 (highest score) = no stress symptoms, 0 (lowest score; most severe effect) = extreme stress symptoms
*line performed better than control (significant at P < 0.11)

Discussion. Overexpression of G3771 caused accelerated flowering and dwarfing in a comparable manner to the overexpression of G3086. Three lines were more drought tolerant than control plants.

Potential applications. Based on the data so far, G3771 may be used to regulate the floral transition and confer drought tolerance in plants, including commercial species.

### Example XIV

### Transformation of Dicots to Produce Increased Biomass, Disease Resistance or Abiotic Stress Tolerance

Crop species including tomato and soybean plants that overexpress any of a considerable number of the transcription factor polypeptides of the invention have been shown experimentally to produce plants with increased drought tolerance and/or biomass in field trials. For example, tomato plants overexpressing the G2153 polypeptide have been found to be larger than wild-type control tomato plants. For example, soy plants overexpressing a number of G481, G682, G867 and G1073 orthologs have been shown to be more drought tolerant than control plants. These observations indicate that these genes, when overexpressed, will result in larger yields than non-transformed plants in both stressed and non-stressed conditions.

Thus, transcription factor polynucleotide sequences listed in the Sequence Listing recombined into, for example, one of the expression vectors of the invention, or another suitable expression vector, may be transformed into a plant for the purpose of modifying plant traits for the purpose of improving yield and/or quality. The expression vector may contain a constitutive, tissue-specific or inducible promoter operably linked to the transcription factor polynucleotide. The cloning vector may be introduced into a variety of plants by means well known in the art such as, for example, direct DNA transfer or Agrobacterium tumefaciens-mediated transformation. It is now routine to produce transgenic plants using most dicot plants (see Weissbach and Weissbach, (1989); Gelvin et al. (1990); Herrera-Estrella et al. (1983); Bevan (1984); and Klee (1985)). Methods for analysis of traits are routine in the art and examples are disclosed above.

Numerous protocols for the transformation of tomato and soy plants have been previously described, and are well known in the art. Gruber et al. (1993), and Glick and Thompson (1993) describe several expression vectors and culture methods that may be used for cell or tissue transformation and subsequent regeneration. For soybean transformation, methods are described by Miki et al. (1993); and U.S. Pat. No. 5,563,055, (Townsend and Thomas), issued Oct. 8, 1996.

There are a substantial number of alternatives to Agrobacterium-mediated transformation protocols, other methods for the purpose of transferring exogenous genes into soybeans or tomatoes. One such method is microprojectile-mediated transformation, in which DNA on the surface of microprojectile particles is driven into plant tissues with a biolistic device (see, for example, Sanford et al. (1987); Christou et al. (1992); Sanford (1993); Klein et al. (1987); U.S. Pat. No. 5,015,580 (Christou et al), issued May 14, 1991; and U.S. Pat. No. 5,322,783 (Tomes et al.), issued Jun. 21, 1994).

Alternatively, sonication methods (see, for example, Zhang et al. (1991)); direct uptake of DNA into protoplasts using CaCl₂ precipitation, polyvinyl alcohol or poly-L-ornithine (Hain et al. (1985); Draper et al. (1982)); liposome or spheroplast fusion (see, for example, Deshayes et al. (1985); Christou et al. (1987)); and electroporation of protoplasts and whole cells and tissues (see, for example, Donn et al. (1990); D'Halluin et al. (1992); and Spencer et al. (1994)) have been used to introduce foreign DNA and expression vectors into plants.

After a plant or plant cell is transformed (and the latter regenerated into a plant), the transformed plant may be crossed with itself or a plant from the same line, a non-transformed or wild-type plant, or another transformed plant from a different transgenic line of plants. Crossing provides the advantages of producing new and often stable transgenic varieties. Genes and the traits they confer that have been introduced into a tomato or soybean line may be moved into distinct line of plants using traditional backcrossing techniques well known in the art. Transformation of tomato plants may be conducted using the protocols of Koomneef et al (1986), and in U.S. Pat. No. 6,613,962, the latter method described in brief here. Eight day old cotyledon explants are precultured for 24 hours in Petri dishes containing a feeder layer of Petunia hybrida suspension cells plated on MS medium with 2% (w/v) sucrose and 0.8% agar supplemented with 10 μM α-naphthalene acetic acid and 4.4 μM 6-benzylaminopurine. The explants are then infected with a diluted overnight culture of Agrobacterium tumefaciens containing an expression vector comprising a polynucleotide of the invention for 5-10 minutes, blotted dry on sterile filter paper and cocultured for 48 hours on the original feeder layer plates. Culture conditions are as described above. Overnight cultures of Agrobacterium tumefaciens are diluted in liquid MS medium with 2% (w/v/) sucrose, pH 5.7) to an OD₆₀₀ of 0.8.

Following cocultivation, the cotyledon explants are transferred to Petri dishes with selective medium comprising MS medium with 4.56 μM zeatin, 67.3 μM vancomycin, 418.9 μM cefotaxime and 171.6 μM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a kanamycin sulfate-containing medium is a positive indication of a successful transformation.

Transformation of soybean plants may be conducted using the methods found in, for example, U.S. Pat. No. 5,563,055 (Townsend et al., issued Oct. 8, 1996), described in brief here. In this method soybean seed is surface sterilized by exposure to chlorine gas evolved in a glass bell jar. Seeds are germinated by plating on 1/10 strength agar solidified medium without plant growth regulators and culturing at 28° C. with a 16 hour day length. After three or four days, seed may be prepared for cocultivation. The seedcoat is removed and the elongating radicle removed 3-4 mm below the cotyledons.

Overnight cultures of Agrobacterium tumefaciens harboring the expression vector comprising a polynucleotide of the invention are grown to log phase, pooled, and concentrated by centrifugation. Inoculations are conducted in batches such that each plate of seed was treated with a newly resuspended pellet of Agrobacterium. The pellets are resuspended in 20 ml inoculation medium. The inoculum is poured into a Petri dish containing prepared seed and the cotyledonary nodes are macerated with a surgical blade. After 30 minutes the explants are transferred to plates of the same medium that has been solidified. Explants are embedded with the adaxial side up and level with the surface of the medium and cultured at 22° C. for three days under white fluorescent light. These plants may then be regenerated according to methods well established in the art, such as by moving the explants after three days to a liquid counter-selection medium (see U.S. Pat. No. 5,563,055).

The explants may then be picked, embedded and cultured in solidified selection medium. After one month on selective media transformed tissue becomes visible as green sectors of regenerating tissue against a background of bleached, less healthy tissue. Explants with green sectors are transferred to an elongation medium. Culture is continued on this medium with transfers to fresh plates every two weeks. When shoots are 0.5 cm in length they may be excised at the base and placed in a rooting medium.

### Example XV

### Transformation of Monocots to Produce Increased Biomass, Disease Resistance or Abiotic Stress Tolerance

Cereal plants such as, but not limited to, corn, wheat, rice, sorghum, or barley, may be transformed with the present polynucleotide sequences, including monocot or dicot-derived sequences such as those presented in the present Tables, cloned into a vector such as pGA643 and containing a kanamycin-resistance marker, and expressed constitutively under, for example, the CaMV 35S or COR15 promoters, or with tissue-specific or inducible promoters. The expression vectors may be one found in the Sequence Listing, or any other suitable expression vector may be similarly used. For example, pMEN020 may be modified to replace the NptII coding region with the BAR gene of Streptomyces hygroscopicus that confers resistance to phosphinothricin. The KpnI and BglII sites of the Bar gene are removed by site-directed mutagenesis with silent codon changes.

The cloning vector may be introduced into a variety of cereal plants by means well known in the art including direct DNA transfer or Agrobacterium tumefaciens-mediated transformation. The latter approach may be accomplished by a variety of means, including, for example, that of U.S. Pat. No. 5,591,616, in which monocotyledon callus is transformed by contacting dedifferentiating tissue with the Agrobacterium containing the cloning vector.

The sample tissues are immersed in a suspension of 3×10⁻⁹ cells of Agrobacterium containing the cloning vector for 3-10 minutes. The callus material is cultured on solid medium at 25° C. in the dark for several days. The calli grown on this medium are transferred to Regeneration medium. Transfers are continued every 2-3 weeks (2 or 3 times) until shoots develop. Shoots are then transferred to Shoot-Elongation medium every 2-3 weeks. Healthy looking shoots are transferred to rooting medium and after roots have developed, the plants are placed into moist potting soil.

The transformed plants are then analyzed for the presence of the NPTII gene/kanamycin resistance by ELISA, using the ELISA NPTII kit from 5Prime-3Prime Inc. (Boulder, Colo.).

It is also routine to use other methods to produce transgenic plants of most cereal crops (Vasil (1994)) such as corn, wheat, rice, sorghum (Cassas et al. (1993)), and barley (Wan and Lemeaux (1994)). DNA transfer methods such as the microprojectile method can be used for corn (Fromm et al. (1990); Gordon-Kamm et al. (1990); Ishida (1990)), wheat (Vasil et al. (1992); Vasil et al. (1993); Weeks et al. (1993)), and rice (Christou (1991); Hiei et al. (1994); Aldemita and Hodges (1996); and Hiei et al. (1997)). For most cereal plants, embryogenic cells derived from immature scutellum tissues are the preferred cellular targets for transformation (Hiei et al. (1997); Vasil (1994)). For transforming corn embryogenic cells derived from immature scutellar tissue using microprojectile bombardment, the A188XB73 genotype is the preferred genotype (Fromm et al. (1990); Gordon-Kamm et al. (1990)). After microprojectile bombardment the tissues are selected on phosphinothricin to identify the transgenic embryogenic cells (Gordon-Kamm et al. (1990)). Transgenic plants are regenerated by standard corn regeneration techniques (Fromm et al. (1990); Gordon-Kamm et al. (1990)).

### Example XVI

### Transcription Factor Expression and Analysis of Disease Resistance or Abiotic Stress Tolerance

Northern blot analysis, RT-PCR or microarray analysis of the regenerated, transformed plants may be used to show expression of a transcription factor polypeptide or the invention and related genes that are capable of inducing disease resistance, abiotic stress tolerance, and/or larger size.

To verify the ability to confer stress resistance, mature plants overexpressing a transcription factor of the invention, or alternatively, seedling progeny of these plants, may be challenged by a stress such as a disease pathogen, drought, heat, cold, high salt, or desiccation. Alternatively, these plants may challenged in a hyperosmotic stress condition that may also measure altered sugar sensing, such as a high sugar condition. By comparing control plants (for example, wild type) and transgenic plants similarly treated, the transgenic plants may be shown to have greater tolerance to the particular stress.

After a dicot plant, monocot plant or plant cell has been transformed (and the latter regenerated into a plant) and shown to have greater size or tolerance to abiotic stress, or produce greater yield relative to a control plant under the stress conditions, the transformed monocot plant may be crossed with itself or a plant from the same line, a non-transformed or wild-type monocot plant, or another transformed monocot plant from a different transgenic line of plants.

These experiments would demonstrate that transcription factor polypeptides of the invention can be identified and shown to confer larger size, greater yield, greater disease resistance and/or abiotic stress tolerance in dicots or monocots, including tolerance or resistance to multiple stresses.

### Example XVII

### Sequences that Confer Significant Improvements to Non-Arabidopsis Species

The function of specific transcription factors of the invention, including closely-related orthologs, have been analyzed and may be further characterized and incorporated into crop plants. The ectopic overexpression of these sequences may be regulated using constitutive, inducible, or tissue specific regulatory elements. Genes that have been examined and have been shown to modify plant traits (including increasing biomass, disease resistance and/or abiotic stress tolerance) encode transcription factor polypeptides found in the Sequence Listing. In addition to these sequences, it is expected that newly discovered polynucleotide and polypeptide sequences closely related to polynucleotide and polypeptide sequences found in the Sequence Listing can also confer alteration of traits in a similar manner to the sequences found in the Sequence Listing, when transformed into a any of a considerable variety of plants of different species, and including dicots and monocots. The polynucleotide and polypeptide sequences derived from monocots (e.g., the rice sequences) may be used to transform both monocot and dicot plants, and those derived from dicots (e.g., the Arabidopsis and soy genes) may be used to transform either group, although it is expected that some of these sequences will function best if the gene is transformed into a plant from the same group as that from which the sequence is derived.

As an example of a first step to determine drought-related tolerance, seeds of these transgenic plants are subjected to germination assays to measure sucrose sensing. Sterile monocot seeds, including, but not limited to, corn, rice, wheat, rye and sorghum, as well as dicots including, but not limited to soybean and alfalfa, are sown on 80% MS medium plus vitamins with 9.4% sucrose; control media lack sucrose. All assay plates are then incubated at 22° C. under 24-hour light, 120-130 μEin/m²/s, in a growth chamber. Evaluation of germination and seedling vigor is then conducted three days after planting. Plants overexpressing sequences of the invention may be found to be more tolerant to high sucrose by having better germination, longer radicles, and more cotyledon expansion. These methods have been used to show that overexpressors of numerous sequences of the invention are involved in sucrose-specific sugar sensing. It is expected that structurally similar orthologs of these sequences, including those found in the Sequence Listing, are also involved in sugar sensing, an indication of altered osmotic stress tolerance.

Plants overexpressing the transcription factor sequences of the invention may also be subjected to soil-based drought assays to identify those lines that are more tolerant to water deprivation than wild-type control plants. A number of the lines of plants overexpressing transcription factor polypeptides of the invention, including newly discovered closely-related species, will be significantly larger and greener, with less wilting or desiccation, than wild-type control plants, particularly after a period of water deprivation is followed by rewatering and a subsequent incubation period. The sequence of the transcription factor may be overexpressed under the regulatory control of constitutive, tissue specific or inducible promoters, or may comprise a GAL4 transactivation domain fused to either the N- or the C terminus of the polypeptide. The results presented in Examples above indicate that these transcription factors may confer disease resistance or abiotic stress tolerance when they are overexpressed under the regulatory control of non-constitutive promoters or a transactivation domain fused to the clade member, without having a significant adverse impact on plant morphology and/or development. The lines that display useful traits may be selected for further study or commercial development.

Monocotyledonous plants, including rice, corn, wheat, rye, sorghum, barley and others, may be transformed with a plasmid containing a transcription factor polynucleotide. The transcription factor gene sequence may include dicot or monocot-derived sequences such as those presented herein. These transcription factor genes may be cloned into an expression vector containing a kanamycin-resistance marker, and then expressed constitutively or in a tissue-specific or inducible manner.

The cloning vector may be introduced into monocots by, for example, means described in the previous Example, including direct DNA transfer or Agrobacterium tumefaciens-mediated transformation. The latter approach may be accomplished by a variety of means, including, for example, that of U.S. Pat. No. 5,591,616, in which monocotyledon callus is transformed by contacting dedifferentiating tissue with the Agrobacterium containing the cloning vector.

The sample tissues are immersed in a suspension of 3×10⁻⁹ cells of Agrobacterium containing the cloning vector for 3-10 minutes. The callus material is cultured on solid medium at 25° C. in the dark for several days. The calli grown on this medium are transferred to Regeneration medium. Transfers are continued every 2-3 weeks (2 or 3 times) until shoots develop. Shoots are then transferred to Shoot-Elongation medium every 2-3 weeks. Healthy looking shoots are transferred to rooting medium and after roots have developed, the plants are placed into moist potting soil.

The transformed plants are then analyzed for the presence of the NPTII gene/kanamycin resistance by ELISA, using the ELISA NPTII kit from 5Prime-3Prime Inc. (Boulder, Colo.).

Northern blot analysis, RT-PCR or microarray analysis of the regenerated, transformed plants may be used to show expression of a transcription factor polypeptide of the invention that is capable of conferring abiotic stress tolerance, disease resistance, or increased size or yield, in the transformed plants.

To verify the ability to confer abiotic stress tolerance, mature plants or seedling progeny of these plants expressing a monocot-derived equivalog gene may be challenged using methods described in the above Examples. By comparing wild type plants and the transgenic plants, the latter are shown be more tolerant to abiotic stress, more resistant to disease, and/or have increased biomass, as compared to wild type control plants similarly treated.

It is expected that the same methods may be applied to identify other useful and valuable sequences of the present transcription factor clades, and the sequences may be derived from a diverse range of species.

### REFERENCES CITED

Abe et al. (1997) Plant Cell 9: 1859-1868
Abe et al. (2003) Plant Cell 15: 63-78
Affolter et al. (1990) Curr. Opin. Cell. Biol. 2: 485-495
Agrios, G. N. (1997) Plant Pathology. 4^{th} edition. (Academic Press, San Diego, New York)
Aldemita and Hodges (1996) Planta 199: 612-617
Alia et al. (1998) Plant J. 16: 155-161
Allen (1998) EMBO J. 17: 5484-5496.
Altschul (1990) J. Mol. Biol. 215: 403-410
Altschul (1993) J. Mol. Evol. 36: 290-300
Anderson and Young (1985) “Quantitative Filter Hybridisation”, In: Hames and Higgins, ed., Nucleic Acid Hybridisation, A Practical Approach. Oxford, IRL Press, 73-111
Anderson et al. (2004) Plant Cell 16: 3460-3479.
Aravind and Landsman (1998) Nucleic Acids Res. 26: 4413-4421
Arents and Moudrianakis (1995) Proc. Natl. Acad. Sci. USA 92: 11170-11174
Atchley and Fitch (1997) Proc. Natl. Acad. Sci. USA 94: 5172-5176
Atchley et al. (1999) J. Mol. Evol. 48: 501-516
Ausubel et al. (1997) Short Protocols in Molecular Biology, John Wiley & Sons, New York, N.Y., unit 7.7
Bailey et al. (2003) Plant Cell 15: 2497-2502
Bairoch et al. (1997) Nucleic Acids Res. 25: 217-221
Bänzinger et al. (2000) Breeding for drought and nitrogen stress tolerance in maize. From theory to practice. (Mexico: CIMMYT (The International Maize and Wheat Improvement Center))
Barthelemy et al. (1996) Biochem. Biophys. Res. Commun. 224: 870-876
Bates et al. (1973) Plant Soil 39: 205-207
Baudino and Cleveland (2001) Mol. Cell. Biol. 21: 691-702
Bechtold and Pelletier (1998) Methods Mol. Biol. 82: 259-266
Berger and Kimmel (1987) “Guide to Molecular Cloning Techniques”, in Methods in Enzymology, vol. 152, Academic Press, Inc., San Diego, Calif.
Berger et al. (1998) Curr. Biol. 8: 421-430
Berrocal-Lobo et al. (2002) Plant J. 29: 23-32
Berrocal-Lobo and Molina (2004) Mol. Plant Microbe Interact. 17: 763-770
Bevan (1984) Nucleic Acids Res. 12: 8711-8721
Bezhani et al. (2001) J. Biol. Chem. 276: 23785-23789
Bhattachaijee et al. (2001) Proc. Natl. Acad. Sci. USA 98: 13790-13795
Bi et al. (1997) J. Biol. Chem. 272: 26562-26572
Bianchi and Beltrame (2000) EMBO Rep. 1: 109-114
Boter (2004) Genes Dev. 18: 1577-1591
Borevitz et al. (2000) Plant Cell 12: 2383-2393
Boss and Thomas (2002) Nature 416: 847-850
Boyer (1995) Annu. Rev. Phytopathol. 33: 251-274.
Brown et al. (2003) Plant Physiol. 132: 1020-1032
Brownlie et al. (1997) Structure 5: 509-520
Bruce et al. (2000) Plant Cell 12: 65-79
Bucher and Trifonov (1988) J. Biomol. Struct. Dyn. 5: 1231-1236
Bucher (1990) J. Mol. Biol. 212: 563-578
Buck and Atchley (2003) J. Mol. Evol. 56: 742-750
Burglin (1997) Nucleic Acids Res. 25: 4173-4180
Burglin (1998) Dev. Genes Evol. 208: 113-116
Byrne (2000) Nature 408: 967-971
Caretti et al. (2003) J. Biol. Chem. 278: 30435-30440
Carre and Kay (1995) Plant Cell 7: 2039-2051
Carroll (2000) Cell 101: 577-580
Carson et al. (1997) Plant J. 12: 1231-1240
Cassas et al. (1993) Proc. Natl. Acad. Sci. USA 90: 11212-11216
Chae et al. (2004) Oncogene 23: 4084-4088
Chakravarthy et al. (2003) Plant Cell 15: 3033-3050
Chang and Liu (1994) J. Biol. Chem. 269: 17893-17898
Chase et al. (1993) Ann. Missouri Bot. Gard. 80: 528-580
Chen et al. (2002a) Plant Cell 14: 559-574.
Chen and Chen (2002) Plant Physiol. 129: 706-716
Cheong et al. (2002) Plant Physiol. 129: 661-677
Cheong et al. (2003) Plant Physiol. 132: 1961-1972
Chini et al. (2004) Plant J. 38: 810-822.
Chinnusamy et al. (2003) Genes Dev. 17: 1043-1054
Chinthapalli et al. (2002) in Reviews in Plant Biochemistry and Biotechnology, Goyal, A. et al (eds.) pp. 143-159
Christou et al. (1987) Proc. Natl. Acad. Sci. USA 84: 3962-3966
Christou (1991) Bio/Technol. 9:957-962
Christou et al. (1992) Plant. J. 2: 275-281
Ciarapica et al. (2003) J. Biol. Chem. 278: 12182-12190
Costa and Dolan (2003) Development 130: 2893-2901
Coupland (1995) Nature 377: 482-483
Coustry et al. (1995) J. Biol. Chem. 270: 468-475
Coustry et al. (1996) J. Biol. Chem. 271: 14485-14491
Coustry et al. (1998) Biochem J. 331(Pt 1): 291-297
Coustry et al. (2001) J. Biol. Chem. 276: 40621-40630.
Crawford et al. (2004) Plant Physiol. 135: 244-253
Crozatier et al. (1996) Curr. Biol. 6: 707-718
Cubas et al. (1999) Plant J. 18: 215-222
Currie (1997) J. Biol. Chem. 272: 30880-30888
Daly et al. (2001) Plant Physiol. 127: 1328-1333
Dang et al. (1992) Proc. Natl. Acad. Sci. USA 89: 599-602
Dang et al. (1996) J. Bacteriol. 178: 1842-1849
de Pater et al. (1996) Nucleic Acids Res. 24: 4624-4631
Dellagi et al. (2000) Mol. Plant Microbe Interact. 13: 1092-110
Deshayes et al. (1985) EMBO J.: 4: 2731-2737
D'Halluin et al. (1992) Plant Cell 4: 1495-1505
Di Cristina et al. (1996) Plant J. 10: 393-402
Doebley and Lukens (1998) Plant Cell 10: 1075-1082
Donn et al. (1990) in Abstracts of VIIth International Congress on Plant Cell and Tissue Culture IAPTC, A2-38: 53
Doolittle, ed (1996) Methods in Enzymology, vol. 266: “Computer Methods for Macromolecular Sequence Analysis” Academic Press, Inc., San Diego, Calif., USA
Draper et al. (1982) Plant Cell Physiol. 23: 451-458
Du and Chen (2000) Plant J. 24: 837-847
Duboule (1994), (ed.) Guidebook to the homeobox genes Oxford University Press, Oxford
Duckett et al. (1994) Development 120: 3247-3255
Ecker (1995) Science 268: 667-675
Eddy (1996) Curr. Opin. Str. Biol. 6: 361-365
Edwards et al. (1998) Plant Physiol. 117: 1015-1022.
Eimert et al. (1995) Plant Cell 7: 1703-1712
Ellenberger et al. (1994) Genes Dev. 8: 970-980
Eulgem et al. (1999) EMBO J. 18: 4689-4699
Eulgem (2000) Trends Plant Sci. 5: 199-206.
Ezcurra et al. (2000) Plant J. 24: 57-66
Fairchild et al. (2000) Genes Dev. 14: 2377-2391
Fairman et al. (1993) Proc. Natl. Acad. Sci. USA 90: 10429-10433
Falvo et al. (1995) Cell 83: 1101-1111
Feng and Doolittle (1987) J. Mol. Evol. 25: 351-360
Ferre-D'Amare et al. (1994) EMBO J. 13: 180-189
Finkelstein et al. (1998) Plant Cell 10: 1043-1054
Fischer and Droge-Laser (2004) Mol. Plant. Microbe Interact. 17: 1162-1171
Fisher and Goding (1992) EMBO J. 11: 4103-4109
Fisher and Caudy (1998) Bioessays 20: 298-306
Forsburg and Guarente (1988) Genes Dev. 3: 1166-117
Forzani et al. (2001) J. Biol. Chem. 276: 16731-16738
Fowler et al. (2002) Plant Cell 14: 1675-1679
Fowler and Thomashow (2002) Plant Cell 14: 1675-1690
Frampton et al. (1991) Protein Eng. 4: 891-901
Frank et al. (2000) Plant Cell 12: 111-124.
Freeling and Hake (1985) Genetics 111: 617-634
Friedrichsen et al. (2002) Genetics 162: 1445-1456
Fromm et al. (1990) Bio/Technol. 8: 833-839
Fu et al. (2001) Plant Cell 13: 1791-1802
Fuji et al. (2000) Nat. Struct. Biol. 7: 889-893
Fujimoto et al. (2000) Plant Cell 12: 393-404
Fujimoto et al. (2004) Plant Mol. Biol. 56: 225-239
Galigniana et al. (1998) Mol. Endocrinol. 12:1903-1913
Galway et al. (1994) Dev. Biol. 166: 740-754
Gampala et al. (2004). International Conference on Arabidopsis Research. Berlin. Abstract # T04-085
Gancedo (1998) Microbiol. Mol. Biol. Rev. 62: 334-361.
Gaxiola et al. (2001) Proc. Natl. Acad. Sci. USA 98: 11444-11449.
Gelinas et al. (1985) Prog. Clin. Biol. Res. 191: 125-139
Gelvin et al. (1990) Plant Molecular Biology Manual, Kluwer Academic Publishers
Gilmour et al. (1998) Plant J. 16: 433-442
Giraudat et al. (1992) Plant Cell 4: 1251-1261
Glick and Thompson, eds. (1993) Methods in Plant Molecular Biology and Biotechnology. CRC Press., Boca Raton, Fla.
Goff et al. (1992) Genes Dev. 6: 864-875
Good and Chen (1996) Biol Signals 5: 163-169
Goodrich et al. (1993) Cell 75: 519-530
Gordon-Kamm et al. (1990) Plant Cell 2: 603-618
Graf (1992) Curr. Opin. Genet. Dev. 2: 249-255.
Grandori et al. (2000) Ann. Rev. Cell. Dev. Biol. 16: 653-699
Grant et al. (2003) Mol. Plant Microbe Interact. 16: 669-680.
Grasser (1995) Plant J. 7: 185-192
Grasser (2003) Plant Mol. Biol. 53: 281-295
Gruber et al. ((1993) in Methods in Plant Molecular Biology and Biotechnology, p. 89-119
Gu et al. (2000) Plant Cell 12: 771-786
Gu et al. (2002) Plant Cell 14: 817-831
Guiltinan et al. (1990) Science 250: 267-271
Guo et al. (2004) Plant Mol. Biol. 55: 607-618.
Gupta et al (1997a) Plant Mol. Biol. 35: 987-992
Gupta et al. (1997b) Plant Mol. Biol. 34: 529-536
Gusmaroli et al. (2001) Gene 264: 173-185
Gusmaroli et al. (2002) Gene 283: 41-48
Haake et al. (2002) Plant Physiol. 130: 639-648
Hain et al. (1985) Mol. Gen. Genet. 199: 161-168
Hall et al. (2000) Plant Physiol. 123: 1449-1458.
Halliday et al. (1999) Proc. Natl. Acad. Sci. USA 96: 5832-5837
Haymes et al. “Nucleic Acid Hybridization: A Practical Approach”, IRL Press, Washington, D.C. (1985)
Hanes and Brent (1989) Cell 57: 1275-1283
Hanes and Brent (1991) Science 251: 426-430
Hao et al. (1998) J. Biol. Chem. 273: 26857-26861
Hao (2002) Biochemistry 41: 4202-4208
Harper (2002) WO0216655
Hasegawa et al. (2000) Annu. Rev. Plant Mol. Plant Physiol. 51: 463-499.
Hatch (1987) Biochim. Biophys. Acta 895: 81-106
Hattori et al. (1992) Genes Dev. 6: 609-618
Hayashi and Scott (1990) Cell 63: 883-894
He et al. (2000) Transgenic Res. 9: 223-227
He et al. (2001) Mol. Plant. Microbe Interact. 14: 1453-1457
Heim et al. (2003) Mol. Biol. Evol. 20: 735-747
Hein (1990) Methods Enzymol. 183: 626-645
Heisler et al. (2001) Development 128: 1089-1098
Hempel (1997) Development 124: 3845-3853
Henikoff and Henikoff (1991) Nucleic Acids Res. 19: 6565-6572
Herrera-Estrella et al. (1983) Nature 303: 209
Hiei et al. (1994) Plant J. 6:271-282
Hiei et al. (1997) Plant Mol. Biol. 35:205-218
Higgins and Sharp (1988) Gene 73: 237-244
Higgins et al. (1996) Methods Enzymol. 266: 383-402
Hirano et al. (2002) Gene 290: 107-114
Hobo et al. (1999) Proc. Natl. Acad. Sci. USA 96: 15348-15353
Hoecker et al. (1995) Genes Dev. 9: 2459-2469
Hsieh et al. (1998) Proc. Natl. Acad. Sci. USA 95: 13965-13970
Hu et al. (2003) Plant Cell 15: 1951-1961
Hu et al. (2004) Cell Res. 14: 8-15
Hung et al. (1998) Plant Physiol. 117: 73-84
Huq and Quail (2002) EMBO J. 21: 2441-2450
Huth et al. (1997) Nat. Struct. Biol. 4: 657-665
Hwang and Goodman (1995) Plant J. 8: 37-43
Ishida (1990) Nature Biotechnol. 14:745-750
Ishiguro and Nakamura (1994) Mol. Gen. Genet. 244: 563-571
Ito et al. (1995) Plant Cell Physiol. 36: 1281-1289
Jaglo et al. (2001) Plant Physiol. 127: 910-917
Jaglo-Ottosen et al. (1998) Science. 280:104-106
Jakoby et al. (2002) Trends Plant Sci. 7: 106-111
Jaglo et al. (2001) Plant Physiol. 127: 910-917
Jang et al. (1997) Plant Cell 9: 5-19
Jofuku et al. (1994) Plant Cell 6: 1211-1225
Johnson and McKnight (1989) Ann. Rev. Biochem. 58: 799-839
Johnson et al. (2002) Plant Cell 14: 1359-1375
Kagaya et al. (1999) Nucleic Acids Res. 27: 470-478
Kaiser et al. (1998) Science 281: 1202-1206
Kashima et al. (1985) Nature 313: 402-404
Kasuga et al. (1999) Nature Biotechnol. 17: 287-291
Kehoe et al. (1994) Plant Cell 6: 1123-1134
Keith et al. (1994) Plant Cell 6: 589-600
Kerstetter et al. (1994) Plant Cell 6: 1877-1887
Kerstetter et al. (1997) Development 124: 3045-3054
Kim and Sheffrey (1990) J. Biol. Chem. 265: 13362-13369
Kim et al. (1996) Mol. Cell. Biol 0.16: 4003-4013
Kim et al. (2001) Plant J. 25: 247-259
Kim et al. (2004) Plant Mol. Biol. 55: 883-904
Kimmel (1987) Methods Enzymol. 152: 507-511
Kirik et at. (2004a) Dev. Biol. 268: 506-513
Kirik et al. (2004b) Plant Mol. Biol. 55: 389-398
Kissinger et al. (1990) Cell 63: 579-590
Klee (1985) Bio/Technology 3: 637-642
Klein et al. (1987) Nature 327: 70-73
Knight (2000a) Int. Rev. Cytol. 195: 269-324.
Koornneef et al (1986) In Tomato Biotechnology: Alan R. Liss, Inc., 169-178
Krizek (1999) Dev. Genet. 25: 224-236
Ku et al. (2000) Proc. Natl. Acad. Sci. USA 97: 9121-9126
Kunst et al. (2000) Biochem Soc. Trans. 28: 651-654.
Kusnetsov et al. (1999) J. Biol. Chem. 274: 36009-36014
Kwak et al. (2005) Science 307: 1111-1113
Kwong (2003) Plant Cell 15: 5-18
Kyozuka and Shimamoto (2002) Plant Cell Physiol. 43: 130-135
Lapik and Kaufman (2003) Plant Cell 15: 1578-1590
Larkin et al. (2003) Ann. Rev. Plant Biol. 54: 403-430
Lebel et al. (1998) Plant J. 16: 223-233
Ledent and Vervoort (2001) Genome Res. 11: 754-770
Lee and Schiefelbein (1999) Cell 99: 473-483
Lee et al. (2002) Genome Res. 12: 493-502
Lee and Schiefelbein (2002) Plant Cell 14: 611-618
Lee et al. (2003) Proc. Natl. Acad. Sci. USA 100: 2152-2156.
Lee et al. (2004) Plant Mol. Biol. 55: 61-81.
Lefstin and Yamamoto (1998) Nature 392: 885-888
Leon-Kloosterziel et al. (1996) Plant Physiol. 110: 233-240
Levens (2003) Genes Dev. 17: 1071-1077
Li et al. (1992) Nucleic Acids Res. 20: 1087-1091
Li et al. (1998) EMBO J. 17: 6300-6315
Lin et al. (1991) Nature 353: 569-571
Lincoln et al. (1990) Plant Cell 2: 1071-1080
Liscum and Reed (2002) Plant Mol. Biol. 49: 387-400
Littlewood and Evan (1998) Helix-Loop-Helix Transcription Factors (New York: Oxford University Press)
Liu and Zhu (1997) Proc. Natl. Acad. Sci. USA 94: 14960-14964
Liu et al. (1999) Eur. J. Biochem. 262: 247-257
Livingston et al. (2004) Economic and policy implications of wind-borne entry of Asian soybean rust into the United States. http://www.ers.usda.gov/Features/SoyBeanRust/
Long et al. (1996) Nature 379: 66-69
Long and Barton (2000) Dev. Biol. 218: 341-353
Lorenzo et al. (2003) Plant Cell 15: 165-178
Lorenzo et al. (2004) Plant Cell 16: 1938-1950
Lotan et al. (1998) Cell 93: 1195-1205.
Loulergue et al. (1998) Gene 225: 47-57
Ludwig et al. (1989) Proc. Natl. Acad. Sci. USA 86: 7092-7096
Ludwig et al. (1990) Cell 62: 849-851
Luerssen et al. (1998) Plant J. 15: 755-764
Luger et al. (1997) Nature 389: 251-260
Lynch et al. (2002) Phytopathol. 92: S33.
Ma et al. (1994) Cell 77: 451-459
Mackay and Crossley (1998) Trends Biochem. Sci. 23: 1-4
Maity and de Crombrugghe (1998) Trends Biochem. Sci. 23: 174-178
Maleck (2000) Nat. Genet. 26: 403-410
Mandel (1992) Nature 360: 273-277
Mandel et al. (1992) Cell 71-133-143
Mantovani (1998) Nucleic Acids Res. 26: 1135-1143
Mantovani (1999) Gene 239: 15-27.
Mare et al. (2004) Plant Mol. Biol. 55: 399-416
Martin and Paz-Ares (1997) Trends Genet. 13: 67-73
Martinez-Garcia and Quail (1999) Plant J. 18: 173-183
Martinez-Garcia et al. (2000) Science 288: 859-863
Masiero et al. (2002) J. Biol. Chem. 277: 26429-26435
Massari and Murre (2000) Mol. Cell. Biol. 20: 429-440
Masucci J. et al. (1996) Development 122: 1253-1260
Mazon et al. (1982) Eur. J. Biochem. 127: 605-608
McCarty et al. (1989) Plant Cell 1: 523-532
McCarty et al. (1991) Cell 66: 895-905
McCue and Hanson (1990) Trends Biotechnol. 8: 358-362
McNabb et al. (1995) Genes Dev. 9: 47-58
McNabb et al. (1997) Mol. Cell. Biol. 17: 7008-7018
Meijer et al. (1996) Plant Mol. Biol. 31: 607-618
Meinke (1992) Science 258: 1647-1650
Meinke et al. (1994) Plant Cell 6: 1049-1064
Merlot et al. (2001) Plant J. 25: 295-303.
Mewes et al. (2002) Nucleic Acids Res. 30: 31-34
Meyers (1995) Molecular Biology and Biotechnology, Wiley VCH, New York, N.Y., p 856-853
Miki et al. (1993) in Methods in Plant Molecular Biology and Biotechnology, p. 67-88, Glick and Thompson, eds., CRC Press, Inc., Boca Raton
Miles et al. (2003) Soybean rust: is the U.S. soybean crop at risk? http://www.apsnet.org/online/feature/rust/
Miyoshi et al. (2003) Plant J. 36: 532-540
Mizukama and Fischer (2000) Proc. Natl. Acad. Sci. USA 97-942-947
Mizukami (2001) Curr Opinion Plant Biol. 4: 533-539
Mohr and Cahill (2001) Functional Plant Biology 30: 461-469
Mount (2001), in Bioinformatics: Sequence and Genome Analysis, Cold Spring Harbor Laboratory Press, Cold Spring Harbor, N.Y., page 543
Müller et al. (2001) Plant J. 28: 169-179
Munkvold (2003) Annu. Rev. Phytopathol. 41: 99-116.
Murre et al. (1989) Cell 56: 777-783
Myers et al. (1986) Science 232: 613-618
Nair and Burley (2000) Nature 404: 715:717-718
Nakshatri (1996) J. Biol. Chem. 271: 28784-28791.
Nambara et al. (1995) Development 121: 629-636
Nandi et al. (2000) Curr. Biol. 10: 215-218
Nath et al. (2003) Science 299: 1404-1407
Nesi et al. (2000) Plant Cell 12: 1863-1878
Ni et al. (1998) Cell 95: 657-667
Nieto-Sotelo and Quail (1994) Biochem. Soc. Symp. 60: 265-275
North Dakota State University Extension Service. (2002). Managing Row Crop Diseases in Drought Years. http://www.ag.ndsu.nodak.edu/drought/ds-10-97.htm
North Dakota State University Extension Service. (2004). Small Grain Diseases: Management of Those More Common and Severe in Dry Years. http://www.ag.ndsu.nodak.edu/drought/ds-01-02.htm
Novillo et al. (2004) Proc. Natl. Acad. Sci. USA 101: 3985-3990
Odell (1985) Nature 313: 810-812
Ohme-Takagi and Shinshi (1995) Plant Cell 7: 173-182
Ohta et al. (2001) Plant Cell 13: 1959-1968
Okamuro et al. (1997) Proc. Natl. Acad. Sci. USA 94: 7076-7081
Olesen and Guarente (1990) Genes Dev. 4: 1714-1729
Onate et al. (1994) Mol. Cell. Biol. 14: 3376-3391
Onate-Sanchez and Singh (2002) Plant Physiol. 128: 1313-1322
Ooms et al. (1993) Plant Physiol. 102: 1185-1191
Palatnik et al. (2003) Nature 425: 257-263
Parcy and Giraudat (1997) Plant J. 11: 693-702
Parcy et al. (1997) Plant Cell 9: 1265-1277
Park (2001) Plant Cell 13: 1035-1046.
Payne et al. (2000) Genetics 156: 1349-1362
Peng et al. (1997) Genes Development 11: 3194-3205)
Peng et al. (1999) Nature: 400: 256-261
Pinkham and Guarente (1985) Mol. Cell. Biol. 5: 3410-3416.
Pnueli et al. (2002) Plant J 31: 319-330
Porra et al. (1989) Biochim. Biophys. Acta 975: 384-394
Pourtau et al. (2004) Planta 219: 765-772
Putterill et al (1995) Cell 80: 847-857
Rajani and Sundaresan (2001) Curr. Biol. 11: 1914-1922
Ratcliffe et al. (1990) Plant Physiol. 126: 122-132
Reeves and Nissen (1990) J. Biol. Chem. 265: 8573-8582
Reeves (2001) Gene 277: 63-81.
Reeves and Beckerbauer (2001) Biochim Biophys Acta 1519: 13-29.
Reidt et al. (2000) Plant J. 21: 401-408
Remm et al. (2001) J. Mol. Biol. 314: 1041-1052
Reuber (1998) Plant J. 16: 473-485.
Riechmann and Meyerowitz (1998) Biol. Chem. 379: 633-646
Riechmann et al. (2000a) Science 290: 2105-2110
Riechmann and Ratcliffe (2000b) Curr. Opin. Plant Biol. 3: 423-434
Rieger et al. (1976) Glossary of Genetics and Cytogenetics: Classical and Molecular, 4th ed., Springer Verlag, Berlin
Rieping and Schoffl (1992) Mol. Gen. Genet. 231: 226-232
Rigaut et al. (1999) Nat. Biotechnol. 17: 1030-1032
Robatzek and Somssich (2002) Genes Dev. 16: 1139-1149
Robinson et al. (2000) Nucleic Acids Res. 28: 4460-4466
Robson et al. (2001) Plant J. 28: 619-631
Rohila et al. (2004) Plant J. 38: 172-181
Romier et al. (2003) J. Biol. Chem. 278: 1336-1345
Rushton et al. (1995) Plant Mol. Biol. 29: 691-702
Rushton et al. (1996) EMBO J. 15: 5690-5700
Sadowski et al. (1988) Nature 335: 563-564
Saijo et al. (2000) Plant J. 23: 319-327.
Sakuma et al. (2002) Biochem. Biophys. Res. Comm. 290: 998-1009
Saleki et al. (1993) Plant Physiol. 101: 839-845
Salsi et al. (2003) J. Biol. Chem. 278: 6642-6650
Sambrook et al. (1989) Molecular Cloning: A Laboratory Manual, 2nd Ed., Cold Spring Harbor Laboratory, Cold Spring Harbor, N.Y.
Sanchez and Cejudo (2003) Plant Physiol. 132: 949-957
Sanders et al. (1999) Plant Cell 11: 691-706
Sanford et al. (1987) Part. Sci. Technol. 5:27-37
Sanford (1993) Methods Enzymol. 217: 483-509
Schaffer et al. (1998) Cell 93: 1219-1229
Schellmann et al. (2002) EMBO J. 21: 5036-5046
Schindler et al. (1993) Plant J. 4: 137-150
Schnittger et al. (1998) Development 125: 2283-2289
Schnittger et al. (1999) Plant Cell 11: 1105-1116
Schoof et al. (2000) Cell 100: 635-644
Sessa et al. (1994) Molecular genetic analysis of plant development and metabolism. (Berlin: Springer Verlag).
Sharp and LeNoble (2002) J. Exp. Bot. 53: 33-37.
Sheen (1999) Ann. Rev. Plant Physiol. Plant Mol. Biol. 50: 187-217
Shimizu et al. (1997) EMBO J. 16: 4689-4697
Shin et al. (2002) Mol Plant Microbe Interact 15: 983-989.
Shirakata et al. (1993) Genes Dev. 7: 2456-2470
Shpaer (1997) Methods Mol. Biol. 70: 173-187
Silver et al. (2003) Mol. Cell. Biol. 23: 5989-5999
Sinha et al. (1996) Mol. Cell. Biol. 16: 328-337
Sivamani et al. (2000) Plant Science 155: 1-9
Smalle et al (1998) Proc. Natl. Acad. Sci. USA. 95:3318-3322
Smeekens (1998) Curr. Opin. Plant Biol. 1: 230-234
Smith et al. (1992) Protein Engineering 5: 35-51
Smolen et al. (2002) Genetics 161: 1235-1246
Soltis et al. (1997) Ann. Missouri Bot. Gard. 84: 1-49
Solano et al. (1998) Genes Dev. 12: 3703-3714
Sonnhammer et al. (1997) Proteins 28: 405-420
Sorensen et al. (2003) Plant J. 33: 413-423
Spencer et al. (1994) Plant Mol. Biol. 24: 51-61
Spollen et al. (2000) Plant Physiol. 122: 967-976.
Stockinger et al. (1997) Proc. Natl. Acad. Sci. USA 94: 1035-1040
Stone et al. (2001) Proc. Natl. Acad. Sci. USA 98: 11806-11811
Surpin et al. (2002) Plant Cell 14 Suppl: S327-S338
Suzuki et al. (1997) Plant Cell 9: 799-807
Suzuki et al. (2001) Plant J. 28: 409-418
Suzuki et al. (2003) Plant Physiol. 132: 1664-1677
Svensson et al. (2003) Arch. Biochem. Biophys. 414: 180-188
Tahtihaiu and Palva (2001) Plant J 26: 461-470.
Tamminen et al. (2001) Plant J. 25: 1-8
Tanimoto et al. (1995) Plant J. 8: 943-948
Tasanen et al. (1992) J. Biol. Chem. 267: 11513-11519
Taylor and Scheuring (1994) Mol. Gen. Genet. 243: 148-157
Tepperman et al. (2001) Proc. Natl. Acad. Sci. USA 98: 9437-9442
Thaler and Bostock (2003) Ecology 85: 48-58.
Thoma (1994) Plant Physiol. 105: 35-45
Thompson et al. (1994) Nucleic Acids Res. 22: 4673-4680
Tiwari et al. (2001) Plant Cell 13: 2809-2822
Tiwari et al. (2003) Plant Cell 15: 533-543
Toledo-Ortiz et al. (2003) Plant Cell 15: 1749-1770
Toumier et al. (2003) FEBS Lett. 550: 149-154
Toyama et al. (1999) Plant Cell Physiol. 40: 1087-1092
Truernit and Sauer (1995) Planta 196: 564-570
Tudge (2000) in The Variety of Life, Oxford University Press, New York, N.Y. pp. 547-606
Ulmasov et al. (1997) Science 276: 1865-1868
Vasil et al. (1992) Bio/Technol 10:667-674
Vasil et al. (1993) Bio/Technol 11:1553-1558
Vasil (1994) Plant Mol. Biol. 25: 925-937
Verslues and Sharp (1999) Plant Physiol. 119: 1349-1360
Vicient et al. (2000) J. Exp. Bot. 51: 995-1003
Vollbrecht et al. (1991) Nature 350: 241-243
Wada et al. (1997) Science 277: 1113-1116
Wada et. al. (2002) Development 129: 5409-5419
Wahl and Berger (1987) Methods Enzymol. 152: 399-407
Wan and Lemeaux (1994) Plant Physiol. 104: 37-48
Wang et al. (1997) Plant Cell 9: 491-507
Wang (1998) Plant J. 16: 515-522
Wanner and Gruissem (1991) Plant Cell 3: 1289-1303
Waterhouse et al. (2001) Trends Plant Sci. 6: 297-301
Waterston et al. (2002) Nature 420: 520-562
Weeks et al. (1993) Plant Physiol. 102:1077-1084
Weigel et al. (1992) Cell 69: 843-859
Weigel (1995) Plant Cell 7: 388-389
Weigel et al. (2000) Plant Physiol. 122: 1003-1013
Weigel and Nilsson (1995) Nature 377: 482-500
Weissbach and Weissbach (1989) Methods for Plant Molecular Biology, Academic Press
Wendler et al. (1997) J. Biol. Chem. 272: 8482-8489
Wesley et al. (2001) Plant J. 27: 581-590
West et al. (1994) Plant Cell 6: 1731-1745
Westhoff and Gowik (2004) Ann. Bot. (London) 93: 13-23
Windhovel (2001) Plant Mol. Biol. 45: 201-214.
Wobus and Weber (1999) Curr. Opin. Plant Biol. 2: 33-38
Wolberger et al. (1991) Cell 67: 517-528
Wrather and Sweets (2004) Aflatoxin in Corn. http://aes.missouri.edu/delta/croppest/aflacorn.stm
Wu et al. (1996) Plant Cell 8: 617-627
Xin and Browse (1998) Proc. Natl. Acad. Sci. USA 95: 7799-7804
Xing et al. (1993) EMBO J. 12: 4647-4655
Xiong et al. (2001a) Genes Dev. 15: 1971-1984.
Xiong and Zhu (2002) Plant Cell Environ. 25: 131-139.
Xiong and Yang (2003) Plant Cell 15: 745-759.
Xu et al. (1996) Plant Physiol. 110: 249-257
Xu et al. (2001) Proc. Natl. Acad. Sci. USA 98: 15089-15094
Yamada et al. (1999a) FEBS Lett. 460: 41-45
Yamada et al. (1999b) Biochem. Biophys. Res. Commun. 261: 614-621
Yamada et al. (2003) Biochem J. 373: 167-178
Yamaguchi-Shinozaki and Shinozaki (1993) Mol. Gen. Genet. 236: 331-340
Yamasaki et al. (2005) Plant Cell 17: 944-956
Yang et al. (1999) Plant J. 18: 141-149
Yi et al. (2004) Plant Physiol. 136: 2862-2874
Yu et al. (2001) Plant Cell 13: 1527-1540
Yun et al. (2003) J. Biol. Chem. 278: 36966-36972
Zhang et al. (1991) Bio/Technology 9: 996-997
Zhang et al. (2002) Planta 215:: 191-194
Zhang et al. (2003) Development 130: 4859-4869
Zhang and Wang (2005) BMC Evol. Biol. 5:1
Zhong and Ye (2001) Plant Phys. 126: 549-563
Zhou et al. (1995a) Nature 376: 771-774
Zhou et al. (1995b) Cell 83: 925-935
Zhou et al. (1997) EMBO J. 16: 3207-3218
Zhou and Lee (1998) J. Natl. Cancer Inst. 90: 381-388
Zhu et al. (1998) Plant Cell 10: 1181-1191
Zou et al. (2004) J. Biol. Chem. 279: 55770-5577

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

1: A transgenic plant transformed with an expression vector comprising a polynucleotide encoding a transcription factor polypeptide;
wherein the transcription factor polypeptide is an AP2/ERF transcription factor comprising an AP2 domain and a VAHD subsequence, and the AP2 domain is at least 68% identical to amino acid coordinates 10-75 of SEQ ID NO: 174;
wherein the expression vector further comprises a stress-inducible promoter operably linked to the polynucleotide; and
wherein the transgenic plant is more tolerant to water deprivation stress than a control plant.

2: The transgenic plant of claim 1, wherein the AP2 domain is at least 79% identical to amino acid coordinates 10-75 of SEQ ID NO: 174.

3: The transgenic plant of claim 1, wherein the stress-inducible promoter comprises SEQ ID NO: 937.

4: A method for producing a transgenic plant that is more tolerant to water deprivation stress than a control plant, said method comprising the steps of:
transforming a target plant with an expression vector comprising a polynucleotide encoding a transcription factor polypeptide;
wherein the transcription factor polypeptide is an AP2/ERF transcription factor comprising an AP2 domain and a VAHD subsequence, and the AP2 domain is at least 68% identical to amino acid coordinates 10-75 of SEQ ID NO: 174; and
wherein the expression vector further comprises a stress-inducible promoter operably linked to the polynucleotide.

5: A transgenic plant transformed with an expression vector comprising a polynucleotide encoding a transcription factor polypeptide;
wherein the transcription factor polypeptide comprises a bHLH domain that is at least 76% identical to amino acid coordinates 307-365 of SEQ ID NO: 292;
wherein the expression vector further comprises a root tissue-specific promoter operably linked to the polynucleotide; and
wherein the transgenic plant flowers earlier than a control plant.

6: The transgenic plant of claim 5, wherein the bHLH domain is at least 88% identical to amino acid coordinates 307-365 of SEQ ID NO: 292.

7: The transgenic plant of claim 5, wherein the root tissue-specific promoter comprises SEQ ID NO: 934.

8: A method for producing a transgenic plant that flowers earlier than a control plant, said method comprising the steps of:
transforming a target plant with an expression vector comprising a polynucleotide encoding a transcription factor polypeptide;
wherein the transcription factor polypeptide comprises a bHLH domain that is at least 76% identical to amino acid coordinates 307-365 of SEQ ID NO: 292;
wherein the expression vector further comprises a root tissue-specific promoter operably linked to the polynucleotide.

9: A transgenic plant transformed with an expression vector comprising a polynucleotide encoding a transcription factor polypeptide;
wherein the transcription factor polypeptide comprises a Myb-related domain that is at least 61% identical to amino acid coordinates 33-77 of SEQ ID NO: 60;
wherein the expression vector further comprises an epidermal tissue-specific promoter operably linked to the polynucleotide; and
wherein the transgenic plant is more tolerant to low nitrogen conditions, osmotic stress or water deprivation than a control plant.

10: The transgenic plant of claim 9, wherein the bHLH domain is at least 70% identical to amino acid coordinates 33-77 of SEQ ID NO: 60.

11: The transgenic plant of claim 9, wherein the epidermal-tissue specific promoter comprises SEQ ID NO: 928 or SEQ ID NO: 933.

12: A method for producing a transgenic plant that is more tolerant to low nitrogen conditions, osmotic stress or water deprivation than a control plant, said method comprising the steps of:
transforming a target plant with an expression vector comprising a polynucleotide encoding a transcription factor polypeptide;
wherein the transcription factor polypeptide comprises a Myb-related domain that is at least 61% identical to amino acid coordinates 33-77 of SEQ ID NO: 60;
wherein the expression vector further comprises a vascular tissue-specific promoter operably linked to the polynucleotide.

13: A transgenic plant transformed with an expression vector comprising a polynucleotide encoding a transcription factor polypeptide;
wherein the transcription factor polypeptide comprises an AT-hook domain that is least 78% identical to amino acid coordinates 63-71 of SEQ ID NO: 114 and a second conserved domain that is least 65% identical to amino acid coordinates 107-204 of SEQ ID NO: 114;
wherein the expression vector further comprises a meristem- or epidermal tissue-specific promoter operably linked to the polynucleotide; and
wherein the transgenic plant is more tolerant to osmotic stress or water deprivation, or has greater biomass, than a control plant.

14: The transgenic plant of claim 13, wherein the second conserved domain is at least 71% identical to amino acid coordinates 107-204 of SEQ ID NO: 114.

15: The transgenic plant of claim 13, wherein the meristem tissue-specific or epidermal tissue-specific promoter comprises SEQ ID NO: 930, SEQ ID NO: 933, or SEQ ID NO: 935.

16: A method for producing a transgenic plant that is more tolerant to osmotic stress or water deprivation than a control plant, or has greater biomass than a control plant, said method comprising the steps of:
transforming a target plant with an expression vector comprising a polynucleotide encoding a transcription factor polypeptide;
wherein the transcription factor polypeptide comprises an AT-hook domain that is least 78% identical to amino acid coordinates 63-71 of SEQ ID NO: 114 and a second conserved domain that is least 65% identical to amino acid coordinates 107-204 of SEQ ID NO: 114;
wherein the expression vector further comprises a meristem- or epidermal tissue-specific promoter operably linked to the polynucleotide.

17: A transgenic plant transformed with an expression vector comprising a polynucleotide;
wherein the polynucleotide encodes a first polypeptide comprising an AT-hook domain that is least 78% identical to amino acid coordinates 63-71 of SEQ ID NO: 114 and a second conserved domain that is least 65% identical to amino acid coordinates 107-204 of SEQ ID NO: 114; and
the polynucleotide also encodes a second polypeptide comprising a B domain that is least 81% identical to amino acid coordinates 20-110 of SEQ ID NO: 2; and
wherein the transgenic plant is later flowering and/or has greater biomass than a control plant.

18: The transgenic plant of claim 17, wherein the first polypeptide comprises SEQ ID NO: 114.

19: The transgenic plant of claim 17, wherein the second polypeptide comprises SEQ ID NO: 2.

20. (canceled)

21: A plant comprising a DNA construct encoding a polypeptide;
(a) wherein the polypeptide has a percent identity with a sequence selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, and 420; and
(b) wherein the polypeptide shares a percent identity with a sequence of (a), or comprises a conserved domain sharing the percent identity with the sequence of (a);
wherein the percent identity is selected from the group consisting of at least 55%, at least 56%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 93%, at least 95%, at least 96%, at least 98%, and 100%;
wherein when the polypeptide is expressed in the plant, said expression confers to the plant a trait that is altered with respect to a control plant;
wherein said trait is selected from the group consisting: altered C/N sensing, altered leaf orientation, upward pointing cotyledons, altered leaf shape, altered leaf shape, broad leaves at later stages, altered root branching, dark green leaf color, decreased ABA sensitivity, decreased anthocyanin, decreased tolerance to NaCl, decreased trichome density, early flowering, glossy leaves, gray leaf color, increased biomass, increased chlorophyll, increased resistance to Botrytis, increased resistance to Erysiphe, increased resistance to Sclerotinia, increased root hair, increased root mass, increased seed number, increased seedling size, increased starch, increased tolerance to cold, increased tolerance to dehydration, increased tolerance to drought, increased tolerance to heat, increased tolerance to hyperosmotic stress, increased tolerance to low nitrogen conditions, increased tolerance to mannitol, increased tolerance to NaCl, increased tolerance to sucrose, increased tolerance to sucrose and mannitol, increased tolerance to sugar, decreased apical dominance, large flower, large leaf size, late flowering, late senescence, pale seed color, photosynthesis rate increased, thicker stem, and trilocular siliques.
